SEQUENCE LISTING (308 sequences)

SEQ ID NO: 1 - protein, 5 aa, synthetic construct
SYAMS

SEQ ID NO: 2 - protein, 5 aa, synthetic construct
DYAMS

SEQ ID NO: 3 - protein, 10 aa, synthetic construct
MKSTRHWIDD

SEQ ID NO: 4 - protein, 11 aa, synthetic construct
QGDSLRSYYAS

SEQ ID NO: 5 - protein, 7 aa, synthetic construct
GKNNRPS

SEQ ID NO: 6 - protein, 11 aa, synthetic construct
QSYDSSSYHYV

SEQ ID NO: 7 - protein, 17 aa, synthetic construct
AISGSGGSTYYADSVKG

SEQ ID NO: 8 - protein, 17 aa, synthetic construct
AIRWHSSWTYYADSVKG

SEQ ID NO: 9 - protein, 17 aa, synthetic construct
AISWHSSWTYYADSVKG

SEQ ID NO: 10 - protein, 17 aa, synthetic construct
AIRGHSSWTYYADSVKG

SEQ ID NO: 11 - protein, 17 aa, synthetic construct
AIRWSSSWTYYADSVKG

SEQ ID NO: 12 - protein, 17 aa, synthetic construct
AIRWHGSWTYYADSVKG

SEQ ID NO: 13 - protein, 17 aa, synthetic construct
AIRWHSGWTYYADSVKG

SEQ ID NO: 14 - protein, 17 aa, synthetic construct
AIRWHSSSTYYADSVKG

SEQ ID NO: 15 - protein, 17 aa, synthetic construct
  UNSURE 6: note = G or S
  UNSURE 7: note = G or S
AIRWHXXWTYYADSVKG

SEQ ID NO: 16 - protein, 1041 aa, synthetic construct
HHHHHHHHHHASEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTGAQPALAGTESPVREEPGEDFPAARRLYWDDLKRKLSEKLDSTDFTGTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 17 - protein, 1041 aa, synthetic construct
HHHHHHHHHHASEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTGAQPALAGTESPAREEPEEDFPAAPRLYWDDLKRKLSEKLDTTDFTSTIKLLNENLYVPREAGSQKDENLALYIENQFREFKLSKVWRDQHFVKIQVKDSAQNSVIIVDKNGGLVYLVENPGGYVAYSKAATVTGKLVHANFGTKKDFEDLDSPVNGSIVIVRAGKITFAEKVANAESLNAIGVLIYMDQTKFPIVKADLSFFGHAHLGTGDPYTPGFPSFNHTQFPPSQSSGLPNIPVQTISRAAAEKLFGNMEGDCPSDWKTDSTCKMVTSENKSVKLTVSNVLKETKILNIFGVIKGFVEPDHYVVVGAQRDAWGPGAAKSSVGTALLLKLAQMFSDMVLKDGFQPSRSIIFASWSAGDFGSVGATEWLEGYLSSLHLKAFTYINLDKAVLGTSNFKVSASPLLYTLIEKTMQDVKHPVTGRSLYQDSNWASKVEKLTLDNAAFPFLAYSGIPAVSFCFCEDTDYPYLGTTMDTYKELVERIPELNKVARAAAEVAGQFVIKLTHDTELNLDYERYNSQLLLFLRDLNQYRADVKEMGLSLQWLYSARGDFFRATSRLTTDFRNAEKRDKFVMKKLNDRVMRVEYYFLSPYVSPKESPFRHVFWGSGSHTLSALLESLKLRRQNNSAFNETLFRNQLALATWTIQGAANALSGDVWDIDNEF

SEQ ID NO: 18 - DNA, 723 bp, Homo sapiens
  source 1..723: mol_type = genomic DNA
gaggtgcagctgctggagtctggcggcggcctggtgcagccaggcggcagcctgcggctgtcctgtgccgcctctggcttcaccttttctagctacgccatgagctgggtgagacaggcccctggcaagggcctggagtgggtgtccgccatctccggatctggaggctctacatactatgccgacagcgtgaagggccggttcaccatcagcagagataactccaagaatacactgtatctgcagatgaacagcctgagggccgaggacaccgccgtgtactattgcgccaagatgaagtccacacgccactggatcgacgattggggccagggcaccctggtgacagtgtcctctggaggaggaggatccggaggaggaggctctggcggcggcggcagctccgagctgacccaggaccctgccgtgagcgtggccctgggccagaccgtgaggatcacatgtcagggcgattccctgcgctcttactatgcctcctggtaccagcagaagccaggacaggcacccgtgctggtcatctacggcaagaacaataggccaagcggcatccccgatcgcttttccggctctagctccggcaataccgcaagcctgaccatcacaggagcacaggcagaggacgaggcagattactattgccagtcttatgatagcagctcttaccattatgtcttcggagggggcaccaaactgactgtcctg

SEQ ID NO: 19 - protein, 241 aa, Homo sapiens
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKMKSTRHWIDDWGQGTLVTVSSGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCQSYDSSSYHYVFGGGTKLTVL

SEQ ID NO: 20 - DNA, 1344 bp, synthetic construct
  source 1..1344: mol_type = other DNA
cagatccagttggtgcagtctggacctgagctgaagaagcctggagagacagtcaagatctcctgcaaggcttctggttataccctcacagactattcaatgcactgggtgaagcaggctccaggaaagggtttaaagtggatgggctggataaacactgagactggtgagacaacatatgcagatgacttcaagggacggattgccttctctttggaaacctctgccagcactgcctatttgcagatcaacaacctcaaaaatgaggacacggctacacatttctgtgctagtcccttaactgggacgagggctatggactactggggtcaaggaacctcagtcaccgtctcctcagctagcactaaaggaccaagcgtgttccctctggctccaagcagcaaatcaacctcaggcggcacagcagcactggggtgtctggtgaaggactacttcccagagcccgtcaccgtgtcatggaacagcggagcactgactagcggagtccacacctttccagccgtgctgcagagctccggactgtactccctgtctagtgtggtcacagtgccttcaagctccctggggactcagacctatatctgcaacgtgaatcacaagccctccaatactaaggtcgacaaacgagtggagcctaagtcttgtgataaaacacatacttgccccccttgtcctgctccaccagccgctgcaccaagcgtgttcctgtttcctccaaagcccaaagacacactgatgatcagcagaactcctgaggtcacctgcgtggtcgtggacgtgtcccacgaggatcccgaagtccagtttaactggtacgtggatggggtcgaagtgcataatgcaaagactaaacctcgggaggaacagttcaactctacctttagagtcgtgagtgtgctgacagtcgtgcaccaggactggctgaacggaaaggagtataagtgcaaagtgtctaataagggcctgcccgcccctatcgagaaaacaattagtaagactaaaggccagccaagggaaccccaggtgtacacactgcccccttgtcgcgaggaaatgacaaagaaccaggtctcactgtggtgtctggtgaaagggttctatccatccgacattgccgtggagtgggaatctaatggacagcccgaaaacaattacaagaccacaccacccatgctggacagcgatggatccttctttctgtattcaaagctgaccgtggataaaagccggtggcagcagggcaatgtcttttcctgctctgtgatgcacgaagccctgcacaaccactacactcagaagtccctgtccctgtctcctggcaaa

SEQ ID NO: 21 - protein, 448 aa, synthetic construct
QIQLVQSGPELKKPGETVKISCKASGYTLTDYSMHWVKQAPGKGLKWMGWINTETGETTYADDFKGRIAFSLETSASTAYLQINNLKNEDTATHFCASPLTGTRAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPPAAAPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPCREEMTKNQVSLWCLVKGFYPSDIAVEWESNGQPENNYKTTPPMLDSDGSFFLYSKLTVDKSRWQQGNVFSCSVMHEALHNHYTQKSLSLSPGK

SEQ ID NO: 22 - DNA, 2091 bp, synthetic construct
  source 1..2091: mol_type = other DNA
cagattcagctggtgcagagcggccccgaactgaaaaaacctggagagactgtgaagatttcctgtaaggcatccggctataccctgaccgactacagcatgcactgggtgaagcaggcaccaggcaagggcctgaagtggatgggctggatcaacaccgagacaggcgagacaacatacgccgacgatttcaagggcaggatcgccttttccctggagacaagcgccagcaccgcctatctgcagatcaacaatctgaagaatgaggacaccgccacacacttctgcgcatctccactgaccggaacaagggccatggattactggggccagggcaccagcgtgacagtgagctccgctagcaccaagggccctagcgtgtttccactggccccctctagcaagtccacctctggaggaacagccgccctgggctgtctggtgaaggattatttccctgagccagtgacagtgagctggaactccggcgccctgacctccggagtgcacacatttcccgccgtgctgcagtcctctggcctgtactctctgagctccgtggtgaccgtgccttctagctccctgggcacccagacatatatctgcaacgtgaatcacaagccaagcaatacaaaggtggacaagcgggtggagcccaagtcctgtgataagacccacacatgcccaccttgtcctgccccacccgccgcagccccaagcgtgttcctgtttcctccaaagcccaaggacaccctgatgatctctaggacccctgaggtgacatgcgtggtggtggacgtgagccacgaggatccagaggtgcagttcaactggtacgtggatggcgtggaggtgcacaatgccaagaccaagccccgggaggagcagttcaacagcacctttagagtggtgtccgtgctgacagtggtgcaccaggactggctgaacggcaaggagtataagtgtaaggtgtccaataagggcctgcccgcccctatcgagaagaccatctctaagacaaagggacagcctagggagccacaggtgtgcaccctgcccccttccagagaggagatgacaaagaaccaggtgagcctgtcctgtgccgtgaagggcttctacccttctgacatcgccgtggagtgggagagcaatggccagccagagaacaattataagaccacaccacccatgctggactctgatggcagcttctttctggtgtccaagctgaccgtggataagtctcggtggcagcagggcaacgtgttttcttgcagcgtgatgcacgaggccctgcacaatcactacacacagaagtccctgtctctgagccctggcaagggaggaggaggatccgcggccgcagaggtgcagctgctggagtctggcggcggcctggtgcagccaggcggcagcctgcggctgtcctgtgccgcctctggcttcaccttttctagctacgccatgagctgggtgagacaggcccctggcaagggcctggagtgggtgtccgccatctccggatctggaggctctacatactatgccgacagcgtgaagggccggttcaccatcagcagagataactccaagaatacactgtatctgcagatgaacagcctgagggccgaggacaccgccgtgtactattgcgccaagatgaagtccacacgccactggatcgacgattggggccagggcaccctggtgacagtgtcctctggaggaggaggatccggaggaggaggctctggcggcggcggcagctccgagctgacccaggaccctgccgtgagcgtggccctgggccagaccgtgaggatcacatgtcagggcgattccctgcgctcttactatgcctcctggtaccagcagaagccaggacaggcacccgtgctggtcatctacggcaagaacaataggccaagcggcatccccgatcgcttttccggctctagctccggcaataccgcaagcctgaccatcacaggagcacaggcagaggacgaggcagattactattgccagtcttatgatagcagctcttaccattatgtcttcggagggggcaccaaactgactgtcctg

SEQ ID NO: 23 - protein, 697 aa, synthetic construct
QIQLVQSGPELKKPGETVKISCKASGYTLTDYSMHWVKQAPGKGLKWMGWINTETGETTYADDFKGRIAFSLETSASTAYLQINNLKNEDTATHFCASPLTGTRAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPPAAAPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVCTLPPSREEMTKNQVSLSCAVKGFYPSDIAVEWESNGQPENNYKTTPPMLDSDGSFFLVSKLTVDKSRWQQGNVFSCSVMHEALHNHYTQKSLSLSPGKGGGGSAAAEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKMKSTRHWIDDWGQGTLVTVSSGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCQSYDSSSYHYVFGGGTKLTVL

SEQ ID NO: 24 - DNA, 657 bp, synthetic construct
  source 1..657: mol_type = other DNA
gatgttgtgatgacccaaactccactctccctgcctgtcagtcttggagatcaagcctccatctcttgcagatctagtcagagccttgtacacattaatggaaacacctatttacattggtacctgcagaagccaggccagtctccaaagctcctgatctacaaagtttccaaccgattttctggggtcccagacaggttcagtggcagtggatcagggacagatttcacactcaagatcagcagagtggaggctgaggatctgggagtttatttctgctctcaaagtacacatgttacgtggacgttcggtggaggcaccaagctggaaatcaaacgtacggtcgccgccccctccgtgtttatttttcctccatctgacgaacagctgaagagtgggaccgcctccgtggtgtgcctgctgaacaatttctacccccgggaggccaaggtgcagtggaaagtcgacaacgctctgcagtctggcaatagtcaggagtcagtgactgaacaggacagcaaggattccacctattctctgagctccaccctgacactgagcaaagcagattacgaaaagcacaaagtctatgcctgcgaagtgacccaccaggggctgagcagtccagtgaccaagtcctttaacaggggagagtgt

SEQ ID NO: 25 - protein, 219 aa, synthetic construct
DVVMTQTPLSLPVSLGDQASISCRSSQSLVHINGNTYLHWYLQKPGQSPKLLIYKVSNRFSGVPDRFSGSGSGTDFTLKISRVEAEDLGVYFCSQSTHVTWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - DNA, 1344 bp, synthetic construct
  source 1..1344: mol_type = other DNA
cagatccagttggtgcagtctggacctgagctgaagaagcctggagagacagtcaagatctcctgcaaggcttctggttataccctcacagactattcaatgcactgggtgaagcaggctccaggaaagggtttaaagtggatgggctggataaacactgagactggtgagacaacatatgcagatgacttcaagggacggattgccttctctttggaaacctctgccagcactgcctatttgcagatcaacaacctcaaaaatgaggacacggctacacatttctgtgctagtcccttaactgggacgagggctatggactactggggtcaaggaacctcagtcaccgtctcctcagctagcactaaaggaccaagcgtgttccctctggctccaagcagcaaatcaacctcaggcggcacagcagcactggggtgtctggtgaaggactacttcccagagcccgtcaccgtgtcatggaacagcggagcactgactagcggagtccacacctttccagccgtgctgcagagctccggactgtactccctgtctagtgtggtcacagtgccttcaagctccctggggactcagacctatatctgcaacgtgaatcacaagccctccaatactaaggtcgacaaacgagtggagcctaagtcttgtgataaaacacatacttgccccccttgtcctgctccaccagccgctgcaccaagcgtgttcctgtttcctccaaagcccaaagacacactgatgatcagcagaactcctgaggtcacctgcgtggtcgtggacgtgtcccacgaggatcccgaagtccagtttaactggtacgtggatggggtcgaagtgcataatgcaaagactaaacctcgggaggaacagttcaactctacctttagagtcgtgagtgtgctgacagtcgtgcaccaggactggctgaacggaaaggagtataagtgcaaagtgtctaataagggcctgcccgcccctatcgagaaaacaattagtaagactaaaggccagccaagggaaccccaggtgtacacactgccccctagtcgcgaggaaatgacaaagaaccaggtctcactgacttgtctggtgaaagggttctatccatccgacattgccgtggagtgggaatctaatggacagcccgaaaacaattacaagaccacaccacccatgctggacagcgatggatccttctttctgtattcaaagctgaccgtggataaaagccggtggcagcagggcaatgtcttttcctgctctgtgatgcacgaagccctgcacaaccactacactcagaagtccctgtccctgtctcctggcaaa

SEQ ID NO: 27 - protein, 448 aa, synthetic construct
QIQLVQSGPELKKPGETVKISCKASGYTLTDYSMHWVKQAPGKGLKWMGWINTETGETTYADDFKGRIAFSLETSASTAYLQINNLKNEDTATHFCASPLTGTRAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPPAAAPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 28 - DNA, 1347 bp, synthetic construct
  source 1..1347: mol_type = other DNA
gaagtccaactgctggaatcgggtggtggtctggtgcaaccgggcggctcgctgcgtctgtcatgtgctgcgtcgggctttacctttagctcttatgcaatgtcctgggtgcgtcaggcaccgggtaaaggtctggaatgggttagcgcaattagtggctccggcggtagcacctattacgccgattctgttaaaggtcgttttaccatctcacgcgacaactcgaaaaatacgctgtatctgcagatgaacagtctgcgcgcagaagataccgctgtctattactgcgcaaaaatgaagtcgactcggcattggatcgacgattggggccaaggtacgctggttacggttagcagcgctagcactaaaggaccaagcgtgttccctctggctccaagcagcaaatcaacctcaggcggcacagcagcactggggtgtctggtgaaggactacttcccagagcccgtcaccgtgtcatggaacagcggagcactgactagcggagtccacacctttccagccgtgctgcagagctccggactgtactccctgtctagtgtggtcacagtgccttcaagctccctggggactcagacctatatctgcaacgtgaatcacaagccctccaatactaaggtcgacaaacgagtggagcctaagtcttgtgataaaacacatacttgccccccttgtcctgcaccagaactgctgggaggacctagcgtgttcctgtttccacccaagccaaaagacaccctgatgattagtagaacccctgaggtcacatgcgtggtcgtggacgtgagccacgaggatccagaagtcaagttcaactggtacgtggatggcgtcgaagtgcataatgctaagacaaaaccccgggaggaacagtacaacagtacctatagagtcgtgtcagtcctgacagtgctgcatcaggactggctgaacgggaaagagtataagtgcaaagtgtccaataaggccctgcccgctcctatcgagaaaactatttctaaggctaaaggccagccaagggaaccccaggtgtacaccctgcctccatcacgcgaggaaatgacaaagaaccaggtcagcctgacttgtctggtgaaagggttctatccatctgacatcgcagtggagtgggaaagtaatggacagcccgaaaacaattacaagaccacaccccctgtgctggactccgatggatctttctttctgtatagcaagctgaccgtggataaatcccggtggcagcagggcaatgtcttttcttgtagtgtgatgcacgaggcactgcacaaccactacacccagaagtcactgtccctgagccccggaaaa

SEQ ID NO: 29 - protein, 449 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKMKSTRHWIDDWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - DNA, 642 bp, synthetic construct
  source 1..642: mol_type = other DNA
tcgtctgaactgacgcaagatccggctgtttctgtggcactgggtcaaacggttcgtatcacgtgtcaaggcgattcactgcgctcctattacgcaagttggtatcagcaaaaaccgggccaggctccggtgctggttatttacggcaaaaacaatcgtccgtccggtatcccggatcgcttttcaggcagctctagtggtaacaccgcgtcgctgaccattacgggtgcgcaagccgaagatgaagcagactattactgccagagctatgacagcagcagctaccattatgtctttggtggtggtacgaaactgaccgtcctggggcagcctaaggccgctccttccgtgacactgttccctccatcctccgaggaactgcaggccaacaaggctaccctcgtgtgcctgatctccgacttttaccctggcgctgtgaccgtggcctggaaggctgatagttctcctgtgaaggccggcgtggaaaccaccacaccttccaagcagtccaacaacaaatacgccgcctcctcctacctgtctctgacccctgaacagtggaagtcccaccggtcctactcttgccaagtgacccatgagggctccaccgtggaaaagaccgtggctcctaccgagtgctct

SEQ ID NO: 31 - protein, 214 aa, synthetic construct
SSELTQDPAVSVALGQTVRITCQGDSLRSYYASWYQQKPGQAPVLVIYGKNNRPSGIPDRFSGSSSGNTASLTITGAQAEDEADYYCQSYDSSSYHYVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 32 - DNA, 723 bp, synthetic construct
  source 1..723: mol_type = other DNA
gaagtccaactgctggaatcgggtggtggtctggtgcaaccgggcggctcgctgcgtctgtcatgtgctgcgtcgggctttacctttagcgattatgcaatgtcctgggtgcgtcaggcaccgggtaaaggtctggaatgggttagcgcaattagatggcatagcagctggacctattacgccgattctgttaaaggtcgttttaccatctcacgcgacaactcgaaaaatacgctgtatctgcagatgaacagtctgcgcgcagaagataccgctgtctattactgcgcaaaaatgaagtcgactcggcattggatcgacgattggggccaaggtacgctggttacggttagcagcggcggtggtggcagcggtggtggcggctcgggtggtggcggctcgtctgaactgacgcaagatccggctgtttctgtggcactgggtcaaacggttcgtatcacgtgtcaaggcgattcactgcgctcctattacgcaagttggtatcagcaaaaaccgggccaggctccggtgctggttatttacggcaaaaacaatcgtccgtccggtatcccggatcgcttttcaggcagctctagtggtaacaccgcgtcgctgaccattacgggtgcgcaagccgaagatgaagcagactattactgccagagctatgacagcagcagctaccattatgtctttggtggtggtacgaaactgaccgtcctg

SEQ ID NO: 33 - protein, 241 aa, synthetic construct
EVQLLESGGGLVQPGGSLRLSCAASGFTFSDYAMSWVRQAPGKGLEWVSAIRWHSSWTYYADSVKGRFTISRDNSKNTLYLQMNSLRAEDTAVYYCAKMKSTRHWIDDWGQGTLVTVSSGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCQSYDSSSYHYVFGGGTKLTVL

SEQ ID NO: 34 - DNA, 723 bp, synthetic construct
  source 1..723: mol_type = other DNA
gaagtccaactgctggaatcgggtggtggtctggtgcaaccgggcggctcgctgcgtctgtcatgtgctgcgtcgggctttacctttagctcttatgcaatgtcctgggtgcgtcaggcaccgggtaaaggtctggaatgggttagcgcaattagatggcatagcagctggacctattacgccgattctgttaaaggtcgttttaccatctcacgcgacaactcgaaaaatacgctgtatctgcagatgaacagtctgcgcgcagaagataccgctgtctattactgcgcaaaaatgaagtcgactcggcattggatcgacgattggggccaaggtacgctggttacggttagcagcggcggtggtggcagcggtggtggcggctcgggtggtggcggctcgtctgaactgacgcaagatccggctgtttctgtggcactgggtcaaacggttcgtatcacgtgtcaaggcgattcactgcgctcctattacgcaagttggtatcagcaaaaaccgggccaggctccggtgctggttatttacggcaaaaacaatcgtccgtccggtatcccggatcgcttttcaggcagctctagtggtaacaccgcgtcgctgaccattacgggtgcgcaagccgaagatgaagcagactattactgccagagctatgacagcagcagctaccattatgtctttggtggtggtacgaaactgaccgtcctg

SEQ ID NO: 35 - protein, 241 aa, synthetic construct
EVQLLESGGGLVQPGGSLRLSCAASGFTFSSYAMSWVRQAPGKGLEWVSAIRWHSSWTYYADSVKGRFTISRDNSKNTLYLQMNSLRAEDTAVYYCAKMKSTRHWIDDWGQGTLVTVSSGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCQSYDSSSYHYVFGGGTKLTVL

SEQ ID NO: 36 - DNA, 723 bp, synthetic construct
  source 1..723: mol_type = other DNA
gaagtccaactgctggaatcgggtggtggtctggtgcaaccgggcggctcgctgcgtctgtcatgtgctgcgtcgggctttacctttagcgattatgcaatgtcctgggtgcgtcaggcaccgggtaaaggtctggaatgggttagcgcaattagttggcatagcagctggacctattacgccgattctgttaaaggtcgttttaccatctcacgcgacaactcgaaaaatacgctgtatctgcagatgaacagtctgcgcgcagaagataccgctgtctattactgcgcaaaaatgaagtcgactcggcattggatcgacgattggggccaaggtacgctggttacggttagcagcggcggtggtggcagcggtggtggcggctcgggtggtggcggctcgtctgaactgacgcaagatccggctgtttctgtggcactgggtcaaacggttcgtatcacgtgtcaaggcgattcactgcgctcctattacgcaagttggtatcagcaaaaaccgggccaggctccggtgctggttatttacggcaaaaacaatcgtccgtccggtatcccggatcgcttttcaggcagctctagtggtaacaccgcgtcgctgaccattacgggtgcgcaagccgaagatgaagcagactattactgccagagctatgacagcagcagctaccattatgtctttggtggtggtacgaaactgaccgtcctg

SEQ ID NO: 37 - protein, 241 aa, synthetic construct
EVQLLESGGGLVQPGGSLRLSCAASGFTFSDYAMSWVRQAPGKGLEWVSAISWHSSWTYYADSVKGRFTISRDNSKNTLYLQMNSLRAEDTAVYYCAKMKSTRHWIDDWGQGTLVTVSSGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCQSYDSSSYHYVFGGGTKLTVL

SEQ ID NO: 38 - DNA, 723 bp, synthetic construct
  source 1..723: mol_type = other DNA
gaagtccaactgctggaatcgggtggtggtctggtgcaaccgggcggctcgctgcgtctgtcatgtgctgcgtcgggctttacctttagcgattatgcaatgtcctgggtgcgtcaggcaccgggtaaaggtctggaatgggttagcgcaattagaggccatagcagctggacctattacgccgattctgttaaaggtcgttttaccatctcacgcgacaactcgaaaaatacgctgtatctgcagatgaacagtctgcgcgcagaagataccgctgtctattactgcgcaaaaatgaagtcgactcggcattggatcgacgattggggccaaggtacgctggttacggttagcagcggcggtggtggcagcggtggtggcggctcgggtggtggcggctcgtctgaactgacgcaagatccggctgtttctgtggcactgggtcaaacggttcgtatcacgtgtcaaggcgattcactgcgctcctattacgcaagttggtatcagcaaaaaccgggccaggctccggtgctggttatttacggcaaaaacaatcgtccgtccggtatcccggatcgcttttcaggcagctctagtggtaacaccgcgtcgctgaccattacgggtgcgcaagccgaagatgaagcagactattactgccagagctatgacagcagcagctaccattatgtctttggtggtggtacgaaactgaccgtcctg

SEQ ID NO: 39 - protein, 241 aa, synthetic construct
EVQLLESGGGLVQPGGSLRLSCAASGFTFSDYAMSWVRQAPGKGLEWVSAIRGHSSWTYYADSVKGRFTISRDNSKNTLYLQMNSLRAEDTAVYYCAKMKSTRHWIDDWGQGTLVTVSSGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCQSYDSSSYHYVFGGGTKLTVL

SEQ ID NO: 40 - DNA, 723 bp, synthetic construct
  source 1..723: mol_type = other DNA
gaagtccaactgctggaatcgggtggtggtctggtgcaaccgggcggctcgctgcgtctgtcatgtgctgcgtcgggctttacctttagcgattatgcaatgtcctgggtgcgtcaggcaccgggtaaaggtctggaatgggttagcgcaattagatggtccagcagctggacctattacgccgattctgttaaaggtcgttttaccatctcacgcgacaactcgaaaaatacgctgtatctgcagatgaacagtctgcgcgcagaagataccgctgtctattactgcgcaaaaatgaagtcgactcggcattggatcgacgattggggccaaggtacgctggttacggttagcagcggcggtggtggcagcggtggtggcggctcgggtggtggcggctcgtctgaactgacgcaagatccggctgtttctgtggcactgggtcaaacggttcgtatcacgtgtcaaggcgattcactgcgctcctattacgcaagttggtatcagcaaaaaccgggccaggctccggtgctggttatttacggcaaaaacaatcgtccgtccggtatcccggatcgcttttcaggcagctctagtggtaacaccgcgtcgctgaccattacgggtgcgcaagccgaagatgaagcagactattactgccagagctatgacagcagcagctaccattatgtctttggtggtggtacgaaactgaccgtcctg

SEQ ID NO: 41 - protein, 241 aa, synthetic construct
EVQLLESGGGLVQPGGSLRLSCAASGFTFSDYAMSWVRQAPGKGLEWVSAIRWSSSWTYYADSVKGRFTISRDNSKNTLYLQMNSLRAEDTAVYYCAKMKSTRHWIDDWGQGTLVTVSSGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCQSYDSSSYHYVFGGGTKLTVL

SEQ ID NO: 42 - DNA, 723 bp, synthetic construct
  source 1..723: mol_type = other DNA
gaagtccaactgctggaatcgggtggtggtctggtgcaaccgggcggctcgctgcgtctgtcatgtgctgcgtcgggctttacctttagcgattatgcaatgtcctgggtgcgtcaggcaccgggtaaaggtctggaatgggttagcgcaattagatggcatggcagctggacctattacgccgattctgttaaaggtcgttttaccatctcacgcgacaactcgaaaaatacgctgtatctgcagatgaacagtctgcgcgcagaagataccgctgtctattactgcgcaaaaatgaagtcgactcggcattggatcgacgattggggccaaggtacgctggttacggttagcagcggcggtggtggcagcggtggtggcggctcgggtggtggcggctcgtctgaactgacgcaagatccggctgtttctgtggcactgggtcaaacggttcgtatcacgtgtcaaggcgattcactgcgctcctattacgcaagttggtatcagcaaaaaccgggccaggctccggtgctggttatttacggcaaaaacaatcgtccgtccggtatcccggatcgcttttcaggcagctctagtggtaacaccgcgtcgctgaccattacgggtgcgcaagccgaagatgaagcagactattactgccagagctatgacagcagcagctaccattatgtctttggtggtggtacgaaactgaccgtcctg

SEQ ID NO: 43 - protein, 241 aa, synthetic construct
EVQLLESGGGLVQPGGSLRLSCAASGFTFSDYAMSWVRQAPGKGLEWVSAIRWHGSWTYYADSVKGRFTISRDNSKNTLYLQMNSLRAEDTAVYYCAKMKSTRHWIDDWGQGTLVTVSSGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCQSYDSSSYHYVFGGGTKLTVL

SEQ ID NO: 44 - DNA, 723 bp, synthetic construct
  source 1..723: mol_type = other DNA
gaagtccaactgctggaatcgggtggtggtctggtgcaaccgggcggctcgctgcgtctgtcatgtgctgcgtcgggctttacctttagcgattatgcaatgtcctgggtgcgtcaggcaccgggtaaaggtctggaatgggttagcgcaattagatggcatagcggttggacctattacgccgattctgttaaaggtcgttttaccatctcacgcgacaactcgaaaaatacgctgtatctgcagatgaacagtctgcgcgcagaagataccgctgtctattactgcgcaaaaatgaagtcgactcggcattggatcgacgattggggccaaggtacgctggttacggttagcagcggcggtggtggcagcggtggtggcggctcgggtggtggcggctcgtctgaactgacgcaagatccggctgtttctgtggcactgggtcaaacggttcgtatcacgtgtcaaggcgattcactgcgctcctattacgcaagttggtatcagcaaaaaccgggccaggctccggtgctggttatttacggcaaaaacaatcgtccgtccggtatcccggatcgcttttcaggcagctctagtggtaacaccgcgtcgctgaccattacgggtgcgcaagccgaagatgaagcagactattactgccagagctatgacagcagcagctaccattatgtctttggtggtggtacgaaactgaccgtcctg

SEQ ID NO: 45 - protein, 241 aa, synthetic construct
EVQLLESGGGLVQPGGSLRLSCAASGFTFSDYAMSWVRQAPGKGLEWVSAIRWHSGWTYYADSVKGRFTISRDNSKNTLYLQMNSLRAEDTAVYYCAKMKSTRHWIDDWGQGTLVTVSSGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCQSYDSSSYHYVFGGGTKLTVL

SEQ ID NO: 46 - DNA, 723 bp, synthetic construct
  source 1..723: mol_type = other DNA
gaagtccaactgctggaatcgggtggtggtctggtgcaaccgggcggctcgctgcgtctgtcatgtgctgcgtcgggctttacctttagcgattatgcaatgtcctgggtgcgtcaggcaccgggtaaaggtctggaatgggttagcgcaattagatggcatagcagcagcacctattacgccgattctgttaaaggtcgttttaccatctcacgcgacaactcgaaaaatacgctgtatctgcagatgaacagtctgcgcgcagaagataccgctgtctattactgcgcaaaaatgaagtcgactcggcattggatcgacgattggggccaaggtacgctggttacggttagcagcggcggtggtggcagcggtggtggcggctcgggtggtggcggctcgtctgaactgacgcaagatccggctgtttctgtggcactgggtcaaacggttcgtatcacgtgtcaaggcgattcactgcgctcctattacgcaagttggtatcagcaaaaaccgggccaggctccggtgctggttatttacggcaaaaacaatcgtccgtccggtatcccggatcgcttttcaggcagctctagtggtaacaccgcgtcgctgaccattacgggtgcgcaagccgaagatgaagcagactattactgccagagctatgacagcagcagctaccattatgtctttggtggtggtacgaaactgaccgtcctg

SEQ ID NO: 47 - protein, 241 aa, synthetic construct
EVQLLESGGGLVQPGGSLRLSCAASGFTFSDYAMSWVRQAPGKGLEWVSAIRWHSSSTYYADSVKGRFTISRDNSKNTLYLQMNSLRAEDTAVYYCAKMKSTRHWIDDWGQGTLVTVSSGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCQSYDSSSYHYVFGGGTKLTVL

SEQ ID NO: 48 - DNA, 2173 bp, synthetic construct
  source 1..2173: mol_type = other DNA
aagcttgccgccaccatggagtggtcttgggtctttctgttctttctgtccgtgacaactggagtgcattctcagatccagttggtgcagtctggacctgagctgaagaagcctggagagacagtcaagatctcctgcaaggcttctggttataccctcacagactattcaatgcactgggtgaagcaggctccaggaaagggtttaaagtggatgggctggataaacactgagactggtgagacaacatatgcagatgacttcaagggacggattgccttctctttggaaacctctgccagcactgcctatttgcagatcaacaacctcaaaaatgaggacacggctacacatttctgtgctagtcccttaactgggacgagggctatggactactggggtcaaggaacctcagtcaccgtctcctcagctagcactaaaggaccaagcgtgttccctctggctccaagcagcaaatcaacctcaggcggcacagcagcactggggtgtctggtgaaggactacttcccagagcccgtcaccgtgtcatggaacagcggagcactgactagcggagtccacacctttccagccgtgctgcagagctccggactgtactccctgtctagtgtggtcacagtgccttcaagctccctggggactcagacctatatctgcaacgtgaatcacaagccctccaatactaaggtcgacaaacgagtggagcctaagtcttgtgataaaacacatacttgccccccttgtcctgctccaccagccgctgcaccaagcgtgttcctgtttcctccaaagcccaaagacacactgatgatcagcagaactcctgaggtcacctgcgtggtcgtggacgtgtcccacgaggatcccgaagtccagtttaactggtacgtggatggggtcgaagtgcataatgcaaagactaaacctcgggaggaacagttcaactctacctttagagtcgtgagtgtgctgacagtcgtgcaccaggactggctgaacggaaaggagtataagtgcaaagtgtctaataagggcctgcccgcccctatcgagaaaacaattagtaagactaaaggccagccaagggaaccccaggtgtgcacactgccccctagtcgcgaggaaatgacaaagaaccaggtctcactgtcttgtgcggtgaaagggttctatccatccgacattgccgtggagtgggaatctaatggacagcccgaaaacaattacaagaccacaccacccatgctggacagcgatggatccttctttctggtttcaaagctgaccgtggataaaagccggtggcagcagggcaatgtcttttcctgctctgtgatgcacgaagccctgcacaaccactacactcagaagtccctgtccctgtctcctggcaaaggcggtggtggcagcgcggccgcagaagtccaactgctggaatcgggtggtggtctggtgcaaccgggcggctcgctgcgtctgtcatgtgctgcgtcgggctttacctttagcgattatgcaatgtcctgggtgcgtcaggcaccgggtaaaggtctggaatgggttagcgcaattagatggcatagcagctggacctattacgccgattctgttaaaggtcgttttaccatctcacgcgacaactcgaaaaatacgctgtatctgcagatgaacagtctgcgcgcagaagataccgctgtctattactgcgcaaaaatgaagtcgactcggcattggatcgacgattggggccaaggtacgctggttacggttagcagcggcggtggtggcagcggtggtggcggctcgggtggtggcggctcgtctgaactgacgcaagatccggctgtttctgtggcactgggtcaaacggttcgtatcacgtgtcaaggcgattcactgcgctcctattacgcaagttggtatcagcaaaaaccgggccaggctccggtgctggttatttacggcaaaaacaatcgtccgtccggtatcccggatcgcttttcaggcagctctagtggtaacaccgcgtcgctgaccattacgggtgcgcaagccgaagatgaagcagactattactgccagagctatgacagcagcagctaccattatgtctttggtggtggtacgaaactgaccgtcctgtgatgaattc

SEQ ID NO: 49 - protein, 697 aa, synthetic construct
QIQLVQSGPELKKPGETVKISCKASGYTLTDYSMHWVKQAPGKGLKWMGWINTETGETTYADDFKGRIAFSLETSASTAYLQINNLKNEDTATHFCASPLTGTRAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPPAAAPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVCTLPPSREEMTKNQVSLSCAVKGFYPSDIAVEWESNGQPENNYKTTPPMLDSDGSFFLVSKLTVDKSRWQQGNVFSCSVMHEALHNHYTQKSLSLSPGKGGGGSAAAEVQLLESGGGLVQPGGSLRLSCAASGFTFSDYAMSWVRQAPGKGLEWVSAIRWHSSWTYYADSVKGRFTISRDNSKNTLYLQMNSLRAEDTAVYYCAKMKSTRHWIDDWGQGTLVTVSSGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCQSYDSSSYHYVFGGGTKLTVL

SEQ ID NO: 50 - DNA, 2091 bp, synthetic construct
  source 1..2091: mol_type = other DNA
cagatccagttggtgcagtctggacctgagctgaagaagcctggagagacagtcaagatctcctgcaaggcttctggttataccctcacagactattcaatgcactgggtgaagcaggctccaggaaagggtttaaagtggatgggctggataaacactgagactggtgagacaacatatgcagatgacttcaagggacggattgccttctctttggaaacctctgccagcactgcctatttgcagatcaacaacctcaaaaatgaggacacggctacacatttctgtgctagtcccttaactgggacgagggctatggactactggggtcaaggaacctcagtcaccgtctcctcagctagcactaaaggaccaagcgtgttccctctggctccaagcagcaaatcaacctcaggcggcacagcagcactggggtgtctggtgaaggactacttcccagagcccgtcaccgtgtcatggaacagcggagcactgactagcggagtccacacctttccagccgtgctgcagagctccggactgtactccctgtctagtgtggtcacagtgccttcaagctccctggggactcagacctatatctgcaacgtgaatcacaagccctccaatactaaggtcgacaaacgagtggagcctaagtcttgtgataaaacacatacttgccccccttgtcctgctccaccagccgctgcaccaagcgtgttcctgtttcctccaaagcccaaagacacactgatgatcagcagaactcctgaggtcacctgcgtggtcgtggacgtgtcccacgaggatcccgaagtccagtttaactggtacgtggatggggtcgaagtgcataatgcaaagactaaacctcgggaggaacagttcaactctacctttagagtcgtgagtgtgctgacagtcgtgcaccaggactggctgaacggaaaggagtataagtgcaaagtgtctaataagggcctgcccgcccctatcgagaaaacaattagtaagactaaaggccagccaagggaaccccaggtgtgcacactgccccctagtcgcgaggaaatgacaaagaaccaggtctcactgtcttgtgcggtgaaagggttctatccatccgacattgccgtggagtgggaatctaatggacagcccgaaaacaattacaagaccacaccacccatgctggacagcgatggatccttctttctggtttcaaagctgaccgtggataaaagccggtggcagcagggcaatgtcttttcctgctctgtgatgcacgaagccctgcacaaccactacactcagaagtccctgtccctgtctcctggcaaaggcggtggtggcagcgcggccgccgaagtccaactgctggaatcgggtggtggtctggtgcaaccgggcggctcgctgcgtctgtcatgtgctgcgtcgggctttacctttagctcttatgcaatgtcctgggtgcgtcaggcaccgggtaaaggtctggaatgggttagcgcaattagatggcatagcagctggacctattacgccgattctgttaaaggtcgttttaccatctcacgcgacaactcgaaaaatacgctgtatctgcagatgaacagtctgcgcgcagaagataccgctgtctattactgcgcaaaaatgaagtcgactcggcattggatcgacgattggggccaaggtacgctggttacggttagcagcggcggtggtggcagcggtggtggcggctcgggtggtggcggctcgtctgaactgacgcaagatccggctgtttctgtggcactgggtcaaacggttcgtatcacgtgtcaaggcgattcactgcgctcctattacgcaagttggtatcagcaaaaaccgggccaggctccggtgctggttatttacggcaaaaacaatcgtccgtccggtatcccggatcgcttttcaggcagctctagtggtaacaccgcgtcgctgaccattacgggtgcgcaagccgaagatgaagcagactattactgccagagctatgacagcagcagctaccattatgtctttggtggtggtacgaaactgaccgtcctg

SEQ ID NO: 51 - protein, 697 aa, synthetic construct
QIQLVQSGPELKKPGETVKISCKASGYTLTDYSMHWVKQAPGKGLKWMGWINTETGETTYADDFKGRIAFSLETSASTAYLQINNLKNEDTATHFCASPLTGTRAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPPAAAPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVCTLPPSREEMTKNQVSLSCAVKGFYPSDIAVEWESNGQPENNYKTTPPMLDSDGSFFLVSKLTVDKSRWQQGNVFSCSVMHEALHNHYTQKSLSLSPGKGGGGSAAAEVQLLESGGGLVQPGGSLRLSCAASGFTFSSYAMSWVRQAPGKGLEWVSAIRWHSSWTYYADSVKGRFTISRDNSKNTLYLQMNSLRAEDTAVYYCAKMKSTRHWIDDWGQGTLVTVSSGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCQSYDSSSYHYVFGGGTKLTVL

SEQ ID NO: 52 - DNA, 2091 bp, synthetic construct
  source 1..2091: mol_type = other DNA
cagatccagttggtgcagtctggacctgagctgaagaagcctggagagacagtcaagatctcctgcaaggcttctggttataccctcacagactattcaatgcactgggtgaagcaggctccaggaaagggtttaaagtggatgggctggataaacactgagactggtgagacaacatatgcagatgacttcaagggacggattgccttctctttggaaacctctgccagcactgcctatttgcagatcaacaacctcaaaaatgaggacacggctacacatttctgtgctagtcccttaactgggacgagggctatggactactggggtcaaggaacctcagtcaccgtctcctcagctagcactaaaggaccaagcgtgttccctctggctccaagcagcaaatcaacctcaggcggcacagcagcactggggtgtctggtgaaggactacttcccagagcccgtcaccgtgtcatggaacagcggagcactgactagcggagtccacacctttccagccgtgctgcagagctccggactgtactccctgtctagtgtggtcacagtgccttcaagctccctggggactcagacctatatctgcaacgtgaatcacaagccctccaatactaaggtcgacaaacgagtggagcctaagtcttgtgataaaacacatacttgccccccttgtcctgctccaccagccgctgcaccaagcgtgttcctgtttcctccaaagcccaaagacacactgatgatcagcagaactcctgaggtcacctgcgtggtcgtggacgtgtcccacgaggatcccgaagtccagtttaactggtacgtggatggggtcgaagtgcataatgcaaagactaaacctcgggaggaacagttcaactctacctttagagtcgtgagtgtgctgacagtcgtgcaccaggactggctgaacggaaaggagtataagtgcaaagtgtctaataagggcctgcccgcccctatcgagaaaacaattagtaagactaaaggccagccaagggaaccccaggtgtgcacactgccccctagtcgcgaggaaatgacaaagaaccaggtctcactgtcttgtgcggtgaaagggttctatccatccgacattgccgtggagtgggaatctaatggacagcccgaaaacaattacaagaccacaccacccatgctggacagcgatggatccttctttctggtttcaaagctgaccgtggataaaagccggtggcagcagggcaatgtcttttcctgctctgtgatgcacgaagccctgcacaaccactacactcagaagtccctgtccctgtctcctggcaaaggcggtggtggcagcgcggccgccgaagtccaactgctggaatcgggtggtggtctggtgcaaccgggcggctcgctgcgtctgtcatgtgctgcgtcgggctttacctttagcgattatgcaatgtcctgggtgcgtcaggcaccgggtaaaggtctggaatgggttagcgcaattagttggcatagcagctggacctattacgccgattctgttaaaggtcgttttaccatctcacgcgacaactcgaaaaatacgctgtatctgcagatgaacagtctgcgcgcagaagataccgctgtctattactgcgcaaaaatgaagtcgactcggcattggatcgacgattggggccaaggtacgctggttacggttagcagcggcggtggtggcagcggtggtggcggctcgggtggtggcggctcgtctgaactgacgcaagatccggctgtttctgtggcactgggtcaaacggttcgtatcacgtgtcaaggcgattcactgcgctcctattacgcaagttggtatcagcaaaaaccgggccaggctccggtgctggttatttacggcaaaaacaatcgtccgtccggtatcccggatcgcttttcaggcagctctagtggtaacaccgcgtcgctgaccattacgggtgcgcaagccgaagatgaagcagactattactgccagagctatgacagcagcagctaccattatgtctttggtggtggtacgaaactgaccgtcctg

SEQ ID NO: 53 - protein, 697 aa, synthetic construct
QIQLVQSGPELKKPGETVKISCKASGYTLTDYSMHWVKQAPGKGLKWMGWINTETGETTYADDFKGRIAFSLETSASTAYLQINNLKNEDTATHFCASPLTGTRAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPPAAAPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVCTLPPSREEMTKNQVSLSCAVKGFYPSDIAVEWESNGQPENNYKTTPPMLDSDGSFFLVSKLTVDKSRWQQGNVFSCSVMHEALHNHYTQKSLSLSPGKGGGGSAAAEVQLLESGGGLVQPGGSLRLSCAASGFTFSDYAMSWVRQAPGKGLEWVSAISWHSSWTYYADSVKGRFTISRDNSKNTLYLQMNSLRAEDTAVYYCAKMKSTRHWIDDWGQGTLVTVSSGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCQSYDSSSYHYVFGGGTKLTVL

SEQ ID NO: 54 - DNA, 2091 bp, synthetic construct
  source 1..2091: mol_type = other DNA
cagatccagttggtgcagtctggacctgagctgaagaagcctggagagacagtcaagatctcctgcaaggcttctggttataccctcacagactattcaatgcactgggtgaagcaggctccaggaaagggtttaaagtggatgggctggataaacactgagactggtgagacaacatatgcagatgacttcaagggacggattgccttctctttggaaacctctgccagcactgcctatttgcagatcaacaacctcaaaaatgaggacacggctacacatttctgtgctagtcccttaactgggacgagggctatggactactggggtcaaggaacctcagtcaccgtctcctcagctagcactaaaggaccaagcgtgttccctctggctccaagcagcaaatcaacctcaggcggcacagcagcactggggtgtctggtgaaggactacttcccagagcccgtcaccgtgtcatggaacagcggagcactgactagcggagtccacacctttccagccgtgctgcagagctccggactgtactccctgtctagtgtggtcacagtgccttcaagctccctggggactcagacctatatctgcaacgtgaatcacaagccctccaatactaaggtcgacaaacgagtggagcctaagtcttgtgataaaacacatacttgccccccttgtcctgctccaccagccgctgcaccaagcgtgttcctgtttcctccaaagcccaaagacacactgatgatcagcagaactcctgaggtcacctgcgtggtcgtggacgtgtcccacgaggatcccgaagtccagtttaactggtacgtggatggggtcgaagtgcataatgcaaagactaaacctcgggaggaacagttcaactctacctttagagtcgtgagtgtgctgacagtcgtgcaccaggactggctgaacggaaaggagtataagtgcaaagtgtctaataagggcctgcccgcccctatcgagaaaacaattagtaagactaaaggccagccaagggaaccccaggtgtgcacactgccccctagtcgcgaggaaatgacaaagaaccaggtctcactgtcttgtgcggtgaaagggttctatccatccgacattgccgtggagtgggaatctaatggacagcccgaaaacaattacaagaccacaccacccatgctggacagcgatggatccttctttctggtttcaaagctgaccgtggataaaagccggtggcagcagggcaatgtcttttcctgctctgtgatgcacgaagccctgcacaaccactacactcagaagtccctgtccctgtctcctggcaaaggcggtggtggcagcgcggccgccgaagtccaactgctggaatcgggtggtggtctggtgcaaccgggcggctcgctgcgtctgtcatgtgctgcgtcgggctttacctttagcgattatgcaatgtcctgggtgcgtcaggcaccgggtaaaggtctggaatgggttagcgcaattagaggccatagcagctggacctattacgccgattctgttaaaggtcgttttaccatctcacgcgacaactcgaaaaatacgctgtatctgcagatgaacagtctgcgcgcagaagataccgctgtctattactgcgcaaaaatgaagtcgactcggcattggatcgacgattggggccaaggtacgctggttacggttagcagcggcggtggtggcagcggtggtggcggctcgggtggtggcggctcgtctgaactgacgcaagatccggctgtttctgtggcactgggtcaaacggttcgtatcacgtgtcaaggcgattcactgcgctcctattacgcaagttggtatcagcaaaaaccgggccaggctccggtgctggttatttacggcaaaaacaatcgtccgtccggtatcccggatcgcttttcaggcagctctagtggtaacaccgcgtcgctgaccattacgggtgcgcaagccgaagatgaagcagactattactgccagagctatgacagcagcagctaccattatgtctttggtggtggtacgaaactgaccgtcctg

SEQ ID NO: 55 - protein, 697 aa, synthetic construct
QIQLVQSGPELKKPGETVKISCKASGYTLTDYSMHWVKQAPGKGLKWMGWINTETGETTYADDFKGRIAFSLETSASTAYLQINNLKNEDTATHFCASPLTGTRAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPPAAAPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVCTLPPSREEMTKNQVSLSCAVKGFYPSDIAVEWESNGQPENNYKTTPPMLDSDGSFFLVSKLTVDKSRWQQGNVFSCSVMHEALHNHYTQKSLSLSPGKGGGGSAAAEVQLLESGGGLVQPGGSLRLSCAASGFTFSDYAMSWVRQAPGKGLEWVSAIRGHSSWTYYADSVKGRFTISRDNSKNTLYLQMNSLRAEDTAVYYCAKMKSTRHWIDDWGQGTLVTVSSGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCQSYDSSSYHYVFGGGTKLTVL

SEQ ID NO: 56 - DNA, 2091 bp, synthetic construct
  source 1..2091: mol_type = other DNA
cagatccagttggtgcagtctggacctgagctgaagaagcctggagagacagtcaagatctcctgcaaggcttctggttataccctcacagactattcaatgcactgggtgaagcaggctccaggaaagggtttaaagtggatgggctggataaacactgagactggtgagacaacatatgcagatgacttcaagggacggattgccttctctttggaaacctctgccagcactgcctatttgcagatcaacaacctcaaaaatgaggacacggctacacatttctgtgctagtcccttaactgggacgagggctatggactactggggtcaaggaacctcagtcaccgtctcctcagctagcactaaaggaccaagcgtgttccctctggctccaagcagcaaatcaacctcaggcggcacagcagcactggggtgtctggtgaaggactacttcccagagcccgtcaccgtgtcatggaacagcggagcactgactagcggagtccacacctttccagccgtgctgcagagctccggactgtactccctgtctagtgtggtcacagtgccttcaagctccctggggactcagacctatatctgcaacgtgaatcacaagccctccaatactaaggtcgacaaacgagtggagcctaagtcttgtgataaaacacatacttgccccccttgtcctgctccaccagccgctgcaccaagcgtgttcctgtttcctccaaagcccaaagacacactgatgatcagcagaactcctgaggtcacctgcgtggtcgtggacgtgtcccacgaggatcccgaagtccagtttaactggtacgtggatggggtcgaagtgcataatgcaaagactaaacctcgggaggaacagttcaactctacctttagagtcgtgagtgtgctgacagtcgtgcaccaggactggctgaacggaaaggagtataagtgcaaagtgtctaataagggcctgcccgcccctatcgagaaaacaattagtaagactaaaggccagccaagggaaccccaggtgtgcacactgccccctagtcgcgaggaaatgacaaagaaccaggtctcactgtcttgtgcggtgaaagggttctatccatccgacattgccgtggagtgggaatctaatggacagcccgaaaacaattacaagaccacaccacccatgctggacagcgatggatccttctttctggtttcaaagctgaccgtggataaaagccggtggcagcagggcaatgtcttttcctgctctgtgatgcacgaagccctgcacaaccactacactcagaagtccctgtccctgtctcctggcaaaggcggtggtggcagcgcggccgccgaagtccaactgctggaatcgggtggtggtctggtgcaaccgggcggctcgctgcgtctgtcatgtgctgcgtcgggctttacctttagcgattatgcaatgtcctgggtgcgtcaggcaccgggtaaaggtctggaatgggttagcgcaattagatggtccagcagctggacctattacgccgattctgttaaaggtcgttttaccatctcacgcgacaactcgaaaaatacgctgtatctgcagatgaacagtctgcgcgcagaagataccgctgtctattactgcgcaaaaatgaagtcgactcggcattggatcgacgattggggccaaggtacgctggttacggttagcagcggcggtggtggcagcggtggtggcggctcgggtggtggcggctcgtctgaactgacgcaagatccggctgtttctgtggcactgggtcaaacggttcgtatcacgtgtcaaggcgattcactgcgctcctattacgcaagttggtatcagcaaaaaccgggccaggctccggtgctggttatttacggcaaaaacaatcgtccgtccggtatcccggatcgcttttcaggcagctctagtggtaacaccgcgtcgctgaccattacgggtgcgcaagccgaagatgaagcagactattactgccagagctatgacagcagcagctaccattatgtctttggtggtggtacgaaactgaccgtcctg

SEQ ID NO: 57 - protein, 697 aa, synthetic construct
QIQLVQSGPELKKPGETVKISCKASGYTLTDYSMHWVKQAPGKGLKWMGWINTETGETTYADDFKGRIAFSLETSASTAYLQINNLKNEDTATHFCASPLTGTRAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPPAAAPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVCTLPPSREEMTKNQVSLSCAVKGFYPSDIAVEWESNGQPENNYKTTPPMLDSDGSFFLVSKLTVDKSRWQQGNVFSCSVMHEALHNHYTQKSLSLSPGKGGGGSAAAEVQLLESGGGLVQPGGSLRLSCAASGFTFSDYAMSWVRQAPGKGLEWVSAIRWSSSWTYYADSVKGRFTISRDNSKNTLYLQMNSLRAEDTAVYYCAKMKSTRHWIDDWGQGTLVTVSSGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCQSYDSSSYHYVFGGGTKLTVL

SEQ ID NO: 58 - DNA, 2091 bp, synthetic construct
  source 1..2091: mol_type = other DNA
cagatccagttggtgcagtctggacctgagctgaagaagcctggagagacagtcaagatctcctgcaaggcttctggttataccctcacagactattcaatgcactgggtgaagcaggctccaggaaagggtttaaagtggatgggctggataaacactgagactggtgagacaacatatgcagatgacttcaagggacggattgccttctctttggaaacctctgccagcactgcctatttgcagatcaacaacctcaaaaatgaggacacggctacacatttctgtgctagtcccttaactgggacgagggctatggactactggggtcaaggaacctcagtcaccgtctcctcagctagcactaaaggaccaagcgtgttccctctggctccaagcagcaaatcaacctcaggcggcacagcagcactggggtgtctggtgaaggactacttcccagagcccgtcaccgtgtcatggaacagcggagcactgactagcggagtccacacctttccagccgtgctgcagagctccggactgtactccctgtctagtgtggtcacagtgccttcaagctccctggggactcagacctatatctgcaacgtgaatcacaagccctccaatactaaggtcgacaaacgagtggagcctaagtcttgtgataaaacacatacttgccccccttgtcctgctccaccagccgctgcaccaagcgtgttcctgtttcctccaaagcccaaagacacactgatgatcagcagaactcctgaggtcacctgcgtggtcgtggacgtgtcccacgaggatcccgaagtccagtttaactggtacgtggatggggtcgaagtgcataatgcaaagactaaacctcgggaggaacagttcaactctacctttagagtcgtgagtgtgctgacagtcgtgcaccaggactggctgaacggaaaggagtataagtgcaaagtgtctaataagggcctgcccgcccctatcgagaaaacaattagtaagactaaaggccagccaagggaaccccaggtgtgcacactgccccctagtcgcgaggaaatgacaaagaaccaggtctcactgtcttgtgcggtgaaagggttctatccatccgacattgccgtggagtgggaatctaatggacagcccgaaaacaattacaagaccacaccacccatgctggacagcgatggatccttctttctggtttcaaagctgaccgtggataaaagccggtggcagcagggcaatgtcttttcctgctctgtgatgcacgaagccctgcacaaccactacactcagaagtccctgtccctgtctcctggcaaaggcggtggtggcagcgcggccgccgaagtccaactgctggaatcgggtggtggtctggtgcaaccgggcggctcgctgcgtctgtcatgtgctgcgtcgggctttacctttagcgattatgcaatgtcctgggtgcgtcaggcaccgggtaaaggtctggaatgggttagcgcaattagatggcatggcagctggacctattacgccgattctgttaaaggtcgttttaccatctcacgcgacaactcgaaaaatacgctgtatctgcagatgaacagtctgcgcgcagaagataccgctgtctattactgcgcaaaaatgaagtcgactcggcattggatcgacgattggggccaaggtacgctggttacggttagcagcggcggtggtggcagcggtggtggcggctcgggtggtggcggctcgtctgaactgacgcaagatccggctgtttctgtggcactgggtcaaacggttcgtatcacgtgtcaaggcgattcactgcgctcctattacgcaagttggtatcagcaaaaaccgggccaggctccggtgctggttatttacggcaaaaacaatcgtccgtccggtatcccggatcgcttttcaggcagctctagtggtaacaccgcgtcgctgaccattacgggtgcgcaagccgaagatgaagcagactattactgccagagctatgacagcagcagctaccattatgtctttggtggtggtacgaaactgaccgtcctg

SEQ ID NO: 59 - protein, 697 aa, synthetic construct
QIQLVQSGPELKKPGETVKISCKASGYTLTDYSMHWVKQAPGKGLKWMGWINTETGETTYADDFKGRIAFSLETSASTAYLQINNLKNEDTATHFCASPLTGTRAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPPAAAPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVCTLPPSREEMTKNQVSLSCAVKGFYPSDIAVEWESNGQPENNYKTTPPMLDSDGSFFLVSKLTVDKSRWQQGNVFSCSVMHEALHNHYTQKSLSLSPGKGGGGSAAAEVQLLESGGGLVQPGGSLRLSCAASGFTFSDYAMSWVRQAPGKGLEWVSAIRWHGSWTYYADSVKGRFTISRDNSKNTLYLQMNSLRAEDTAVYYCAKMKSTRHWIDDWGQGTLVTVSSGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCQSYDSSSYHYVFGGGTKLTVL

SEQ ID NO: 60 - DNA, 2091 bp, synthetic construct
  source 1..2091: mol_type = other DNA
cagatccagttggtgcagtctggacctgagctgaagaagcctggagagacagtcaagatctcctgcaaggcttctggttataccctcacagactattcaatgcactgggtgaagcaggctccaggaaagggtttaaagtggatgggctggataaacactgagactggtgagacaacatatgcagatgacttcaagggacggattgccttctctttggaaacctctgccagcactgcctatttgcagatcaacaacctcaaaaatgaggacacggctacacatttctgtgctagtcccttaactgggacgagggctatggactactggggtcaaggaacctcagtcaccgtctcctcagctagcactaaaggaccaagcgtgttccctctggctccaagcagcaaatcaacctcaggcggcacagcagcactggggtgtctggtgaaggactacttcccagagcccgtcaccgtgtcatggaacagcggagcactgactagcggagtccacacctttccagccgtgctgcagagctccggactgtactccctgtctagtgtggtcacagtgccttcaagctccctggggactcagacctatatctgcaacgtgaatcacaagccctccaatactaaggtcgacaaacgagtggagcctaagtcttgtgataaaacacatacttgccccccttgtcctgctccaccagccgctgcaccaagcgtgttcctgtttcctccaaagcccaaagacacactgatgatcagcagaactcctgaggtcacctgcgtggtcgtggacgtgtcccacgaggatcccgaagtccagtttaactggtacgtggatggggtcgaagtgcataatgcaaagactaaacctcgggaggaacagttcaactctacctttagagtcgtgagtgtgctgacagtcgtgcaccaggactggctgaacggaaaggagtataagtgcaaagtgtctaataagggcctgcccgcccctatcgagaaaacaattagtaagactaaaggccagccaagggaaccccaggtgtgcacactgccccctagtcgcgaggaaatgacaaagaaccaggtctcactgtcttgtgcggtgaaagggttctatccatccgacattgccgtggagtgggaatctaatggacagcccgaaaacaattacaagaccacaccacccatgctggacagcgatggatccttctttctggtttcaaagctgaccgtggataaaagccggtggcagcagggcaatgtcttttcctgctctgtgatgcacgaagccctgcacaaccactacactcagaagtccctgtccctgtctcctggcaaaggcggtggtggcagcgcggccgccgaagtccaactgctggaatcgggtggtggtctggtgcaaccgggcggctcgctgcgtctgtcatgtgctgcgtcgggctttacctttagcgattatgcaatgtcctgggtgcgtcaggcaccgggtaaaggtctggaatgggttagcgcaattagatggcatagcggttggacctattacgccgattctgttaaaggtcgttttaccatctcacgcgacaactcgaaaaatacgctgtatctgcagatgaacagtctgcgcgcagaagataccgctgtctattactgcgcaaaaatgaagtcgactcggcattggatcgacgattggggccaaggtacgctggttacggttagcagcggcggtggtggcagcggtggtggcggctcgggtggtggcggctcgtctgaactgacgcaagatccggctgtttctgtggcactgggtcaaacggttcgtatcacgtgtcaaggcgattcactgcgctcctattacgcaagttggtatcagcaaaaaccgggccaggctccggtgctggttatttacggcaaaaacaatcgtccgtccggtatcccggatcgcttttcaggcagctctagtggtaacaccgcgtcgctgaccattacgggtgcgcaagccgaagatgaagcagactattactgccagagctatgacagcagcagctaccattatgtctttggtggtggtacgaaactgaccgtcctg

SEQ ID NO: 61 - protein, 697 aa, synthetic construct
QIQLVQSGPELKKPGETVKISCKASGYTLTDYSMHWVKQAPGKGLKWMGWINTETGETTYADDFKGRIAFSLETSASTAYLQINNLKNEDTATHFCASPLTGTRAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPPAAAPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVCTLPPSREEMTKNQVSLSCAVKGFYPSDIAVEWESNGQPENNYKTTPPMLDSDGSFFLVSKLTVDKSRWQQGNVFSCSVMHEALHNHYTQKSLSLSPGKGGGGSAAAEVQLLESGGGLVQPGGSLRLSCAASGFTFSDYAMSWVRQAPGKGLEWVSAIRWHSGWTYYADSVKGRFTISRDNSKNTLYLQMNSLRAEDTAVYYCAKMKSTRHWIDDWGQGTLVTVSSGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCQSYDSSSYHYVFGGGTKLTVL

SEQ ID NO: 62 - DNA, 2091 bp, synthetic construct
  source 1..2091: mol_type = other DNA
cagatccagttggtgcagtctggacctgagctgaagaagcctggagagacagtcaagatctcctgcaaggcttctggttataccctcacagactattcaatgcactgggtgaagcaggctccaggaaagggtttaaagtggatgggctggataaacactgagactggtgagacaacatatgcagatgacttcaagggacggattgccttctctttggaaacctctgccagcactgcctatttgcagatcaacaacctcaaaaatgaggacacggctacacatttctgtgctagtcccttaactgggacgagggctatggactactggggtcaaggaacctcagtcaccgtctcctcagctagcactaaaggaccaagcgtgttccctctggctccaagcagcaaatcaacctcaggcggcacagcagcactggggtgtctggtgaaggactacttcccagagcccgtcaccgtgtcatggaacagcggagcactgactagcggagtccacacctttccagccgtgctgcagagctccggactgtactccctgtctagtgtggtcacagtgccttcaagctccctggggactcagacctatatctgcaacgtgaatcacaagccctccaatactaaggtcgacaaacgagtggagcctaagtcttgtgataaaacacatacttgccccccttgtcctgctccaccagccgctgcaccaagcgtgttcctgtttcctccaaagcccaaagacacactgatgatcagcagaactcctgaggtcacctgcgtggtcgtggacgtgtcccacgaggatcccgaagtccagtttaactggtacgtggatggggtcgaagtgcataatgcaaagactaaacctcgggaggaacagttcaactctacctttagagtcgtgagtgtgctgacagtcgtgcaccaggactggctgaacggaaaggagtataagtgcaaagtgtctaataagggcctgcccgcccctatcgagaaaacaattagtaagactaaaggccagccaagggaaccccaggtgtgcacactgccccctagtcgcgaggaaatgacaaagaaccaggtctcactgtcttgtgcggtgaaagggttctatccatccgacattgccgtggagtgggaatctaatggacagcccgaaaacaattacaagaccacaccacccatgctggacagcgatggatccttctttctggtttcaaagctgaccgtggataaaagccggtggcagcagggcaatgtcttttcctgctctgtgatgcacgaagccctgcacaaccactacactcagaagtccctgtccctgtctcctggcaaaggcggtggtggcagcgcggccgccgaagtccaactgctggaatcgggtggtggtctggtgcaaccgggcggctcgctgcgtctgtcatgtgctgcgtcgggctttacctttagcgattatgcaatgtcctgggtgcgtcaggcaccgggtaaaggtctggaatgggttagcgcaattagatggcatagcagcagcacctattacgccgattctgttaaaggtcgttttaccatctcacgcgacaactcgaaaaatacgctgtatctgcagatgaacagtctgcgcgcagaagataccgctgtctattactgcgcaaaaatgaagtcgactcggcattggatcgacgattggggccaaggtacgctggttacggttagcagcggcggtggtggcagcggtggtggcggctcgggtggtggcggctcgtctgaactgacgcaagatccggctgtttctgtggcactgggtcaaacggttcgtatcacgtgtcaaggcgattcactgcgctcctattacgcaagttggtatcagcaaaaaccgggccaggctccggtgctggttatttacggcaaaaacaatcgtccgtccggtatcccggatcgcttttcaggcagctctagtggtaacaccgcgtcgctgaccattacgggtgcgcaagccgaagatgaagcagactattactgccagagctatgacagcagcagctaccattatgtctttggtggtggtacgaaactgaccgtcctg

SEQ ID NO: 63 - protein, 697 aa, synthetic construct
QIQLVQSGPELKKPGETVKISCKASGYTLTDYSMHWVKQAPGKGLKWMGWINTETGETTYADDFKGRIAFSLETSASTAYLQINNLKNEDTATHFCASPLTGTRAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPPAAAPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVCTLPPSREEMTKNQVSLSCAVKGFYPSDIAVEWESNGQPENNYKTTPPMLDSDGSFFLVSKLTVDKSRWQQGNVFSCSVMHEALHNHYTQKSLSLSPGKGGGGSAAAEVQLLESGGGLVQPGGSLRLSCAASGFTFSDYAMSWVRQAPGKGLEWVSAIRWHSSSTYYADSVKGRFTISRDNSKNTLYLQMNSLRAEDTAVYYCAKMKSTRHWIDDWGQGTLVTVSSGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCQSYDSSSYHYVFGGGTKLTVL

SEQ ID NO: 64 - protein, 119 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKMKSTRHWIDDWGQGTLVTVSS

SEQ ID NO: 65 - protein, 108 aa, synthetic construct
SSELTQDPAVSVALGQTVRITCQGDSLRSYYASWYQQKPGQAPVLVIYGKNNRPSGIPDRFSGSSSGNTASLTITGAQAEDEADYYCQSYDSSSYHYVFGGGTKLTVL

SEQ ID NO: 66 - protein, 119 aa, synthetic construct
EVQLLESGGGLVQPGGSLRLSCAASGFTFSDYAMSWVRQAPGKGLEWVSAIRWHSSWTYYADSVKGRFTISRDNSKNTLYLQMNSLRAEDTAVYYCAKMKSTRHWIDDWGQGTLVTVSS

SEQ ID NO: 67 - protein, 119 aa, synthetic construct
EVQLLESGGGLVQPGGSLRLSCAASGFTFSSYAMSWVRQAPGKGLEWVSAIRWHSSWTYYADSVKGRFTISRDNSKNTLYLQMNSLRAEDTAVYYCAKMKSTRHWIDDWGQGTLVTVSS

SEQ ID NO: 68 - protein, 119 aa, synthetic construct
EVQLLESGGGLVQPGGSLRLSCAASGFTFSDYAMSWVRQAPGKGLEWVSAISWHSSWTYYADSVKGRFTISRDNSKNTLYLQMNSLRAEDTAVYYCAKMKSTRHWIDDWGQGTLVTVSS

SEQ ID NO: 69 - protein, 119 aa, synthetic construct
EVQLLESGGGLVQPGGSLRLSCAASGFTFSDYAMSWVRQAPGKGLEWVSAIRGHSSWTYYADSVKGRFTISRDNSKNTLYLQMNSLRAEDTAVYYCAKMKSTRHWIDDWGQGTLVTVSS

SEQ ID NO: 70 - protein, 119 aa, synthetic construct
EVQLLESGGGLVQPGGSLRLSCAASGFTFSDYAMSWVRQAPGKGLEWVSAIRWSSSWTYYADSVKGRFTISRDNSKNTLYLQMNSLRAEDTAVYYCAKMKSTRHWIDDWGQGTLVTVSS

SEQ ID NO: 71 - protein, 119 aa, synthetic construct
EVQLLESGGGLVQPGGSLRLSCAASGFTFSDYAMSWVRQAPGKGLEWVSAIRWHGSWTYYADSVKGRFTISRDNSKNTLYLQMNSLRAEDTAVYYCAKMKSTRHWIDDWGQGTLVTVSS

SEQ ID NO: 72 - protein, 119 aa, synthetic construct
EVQLLESGGGLVQPGGSLRLSCAASGFTFSDYAMSWVRQAPGKGLEWVSAIRWHSGWTYYADSVKGRFTISRDNSKNTLYLQMNSLRAEDTAVYYCAKMKSTRHWIDDWGQGTLVTVSS

SEQ ID NO: 73 - protein, 119 aa, synthetic construct
EVQLLESGGGLVQPGGSLRLSCAASGFTFSDYAMSWVRQAPGKGLEWVSAIRWHSSSTYYADSVKGRFTISRDNSKNTLYLQMNSLRAEDTAVYYCAKMKSTRHWIDDWGQGTLVTVSS

SEQ ID NO: 74 - protein, 4 aa, synthetic construct
GGGS

SEQ ID NO: 75 - protein, 5 aa, synthetic construct
GGGGS

SEQ ID NO: 76 - protein, 11 aa, synthetic construct
APGSYTGSAPG

SEQ ID NO: 77 - protein, 4 aa, synthetic construct
GGGG

SEQ ID NO: 78 - protein, 15 aa, synthetic construct
GGGGSGGGGSGGGGS

SEQ ID NO: 79 - protein, 8 aa, synthetic construct
GGGGSAAA

SEQ ID NO: 80 - DNA, 699 bp, synthetic construct
  source 1..699: mol_type = other DNA
gaagtccaactgctggaatcgggtggtggtctggtgcaaccgggcggctcgctgcgtctgtcatgtgctgcgtcgggctttacctttagctcttatgcaatgtcctgggtgcgtcaggcaccgggtaaaggtctggaatgggttagcgcaattagtggctccggcggtagcacctattacgccgattctgttaaaggtcgttttaccatctcacgcgacaactcgaaaaatacgctgtatctgcagatgaacagtctgcgcgcagaagataccgctgtctattactgcgcaaaaatgaagtcgactcggcattggatcgacgattggggccaaggtacgctggttacggttagcagcgctagcactaaaggaccaagcgtgttccctctggctccaagcagcaaatcaacctcaggcggcacagcagcactggggtgtctggtgaaggactacttcccagagcccgtcaccgtgtcatggaacagcggagcactgactagcggagtccacacctttccagccgtgctgcagagctccggactgtactccctgtctagtgtggtcacagtgccttcaagctccctggggactcagacctatatctgcaacgtgaatcacaagccctccaatactaaggtcgacaaacgagtggagcctaagtcttgtgataaaacacatactcaccatcatcatcatcat

SEQ ID NO: 81 - protein, 233 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKMKSTRHWIDDWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTHHHHHH

SEQ ID NO: 82 - DNA, 699 bp, synthetic construct
  source 1..699: mol_type = other DNA
gaagtccaactgctggaatcgggtggtggtctggtgcaaccgggcggctcgctgcgtctgtcatgtgctgcgtcgggctttacctttagcgattatgcaatgtcctgggtgcgtcaggcaccgggtaaaggtctggaatgggttagcgcaattagatggcatagcagctggacctattacgccgattctgttaaaggtcgttttaccatctcacgcgacaactcgaaaaatacgctgtatctgcagatgaacagtctgcgcgcagaagataccgctgtctattactgcgcaaaaatgaagtcgactcggcattggatcgacgattggggccaaggtacgctggttacggttagcagcgctagcactaaaggaccaagcgtgttccctctggctccaagcagcaaatcaacctcaggcggcacagcagcactggggtgtctggtgaaggactacttcccagagcccgtcaccgtgtcatggaacagcggagcactgactagcggagtccacacctttccagccgtgctgcagagctccggactgtactccctgtctagtgtggtcacagtgccttcaagctccctggggactcagacctatatctgcaacgtgaatcacaagccctccaatactaaggtcgacaaacgagtggagcctaagtcttgtgataaaacacatactcaccatcatcatcatcat

SEQ ID NO: 83 - protein, 233 aa, synthetic construct
EVQLLESGGGLVQPGGSLRLSCAASGFTFSDYAMSWVRQAPGKGLEWVSAIRWHSSWTYYADSVKGRFTISRDNSKNTLYLQMNSLRAEDTAVYYCAKMKSTRHWIDDWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTHHHHHH

SEQ ID NO: 84 - DNA, 1959 bp, synthetic construct
  source 1..1959: mol_type = other DNA
catcatcatcatcatcatgctagcggggcccagccggcccgcttatattgggatgacctgaagagaaagttgtcggagaaactggacagcacagacttcaccggcaccatcaagctgctgaatgaaaattcatatgtccctcgtgaggctggatctcaaaaagatgaaaatcttgcgttgtatgttgaaaatcaatttcgtgaatttaaactcagcaaagtctggcgtgatcaacattttgttaagattcaggtcaaagacagcgctcaaaactcggtgatcatagttgataagaacggtagacttgtttacctggtggagaatcctgggggttatgtggcgtatagtaaggctgcaacagttactggtaaactggtccatgctaattttggtactaaaaaagattttgaggatttatacactcctgtgaatggatctatagtgattgtcagagcagggaaaatcacctttgcagaaaaggttgcaaatgctgaaagcttaaatgcaattggtgtgttgatatacatggaccagactaaatttcccattgttaacgcagaactttcattctttggacatgctcatctggggacaggtgacccttacacacctggattcccttccttcaatcacactcagtttccaccatctcggtcatcaggattgcctaatatacctgtccagacaatctccagagctgctgcagaaaagctgtttgggaatatggaaggagactgtccctctgactggaaaacagactctacatgtaggatggtaacctcagaaagcaagaatgtgaagctcactgtgagcaatgtgctgaaagagataaaaattcttaacatctttggagttattaaaggctttgtagaaccagatcactatgttgtagttggggcccagagagatgcatggggccctggagctgcaaaatccggtgtaggcacagctctcctattgaaacttgcccagatgttctcagatatggtcttaaaagatgggtttcagcccagcagaagcattatctttgccagttggagtgctggagactttggatcggttggtgccactgaatggctagagggatacctttcgtccctgcatttaaaggctttcacttatattaatctggataaagcggttcttggtaccagcaacttcaaggtttctgccagcccactgttgtatacgcttattgagaaaacaatgcaaaatgtgaagcatccggttactgggcaatttctatatcaggacagcaactgggccagcaaagttgagaaactcactttagacaatgctgctttccctttccttgcatattctggaatcccagcagtttctttctgtttttgcgaggacacagattatccttatttgggtaccaccatggacacctataaggaactgattgagaggattcctgagttgaacaaagtggcacgagcagctgcagaggtcgctggtcagttcgtgattaaactaacccatgatgttgaattgaacctggactatgagaggtacaacagccaactgctttcatttgtgagggatctgaaccaatacagagcagacataaaggaaatgggcctgagtttacagtggctgtattctgctcgtggagacttcttccgtgctacttccagactaacaacagatttcgggaatgctgagaaaacagacagatttgtcatgaagaaactcaatgatcgtgtcatgagagtggagtatcacttcctctctccctacgtatctccaaaagagtctcctttccgacatgtcttctggggctccggctctcacacgctgccagctttactggagaacttgaaactgcgtaaacaaaataacggtgcttttaatgaaacgctgttcagaaaccagttggctctagctacttggactattcagggagctgcaaatgccctctctggtgacgtttgggacattgacaatgagttt

SEQ ID NO: 85 - protein, 653 aa, synthetic construct
HHHHHHASGAQPARLYWDDLKRKLSEKLDSTDFTGTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 86 - DNA, 990 bp, synthetic construct
  source 1..990: mol_type = other DNA
gctagcaccaagggcccaagcgtgtttccactggccccctctagcaagtccacctctggaggaacagccgccctgggctgtctggtgaaggattatttcccagagcccgtgacagtgagctggaactccggcgccctgacctccggagtgcacacatttcccgccgtgctgcagtcctctggcctgtactctctgagctccgtggtgaccgtgccttctagctccctgggcacccagacatatatctgcaacgtgaatcacaagcccagcaatacaaaggtggacaagcgggtggagcctaagtcctgtgataagacccacacatgccccccttgtcctgcaccagagctgctgggaggacctagcgtgttcctgtttccacccaagccaaaggacaccctgatgatctctaggacccctgaggtgacatgcgtggtggtggacgtgagccacgaggatccagaggtgaagttcaactggtacgtggatggcgtggaggtgcacaatgccaagaccaagccccgggaggagcagtacaacagcacctatagagtggtgtccgtgctgacagtgctgcaccaggactggctgaacggcaaggagtataagtgtaaggtgtccaataaggccctgcccgcccctatcgagaagaccatctctaaggcaaagggacagcctagggagccacaggtgtgcaccctgcctccatccagagaggagatgacaaagaaccaggtgagcctgtcctgtgccgtgaagggcttctacccatctgacatcgccgtggagtgggagagcaatggccagcccgagaacaattataagaccacaccccctgtgctggactctgatggcagcttctttctggtgtccaagctgaccgtggataagtctcggtggcagcagggcaacgtgttttcttgcagcgtgatgcacgaggccctgcacaatcactacacacagaagtccctgtctctgagcccaggcaag

SEQ ID NO: 87 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 88 - DNA, 1737 bp, synthetic construct
  source 1..1737: mol_type = other DNA
gctagcaccaagggcccatccgtgtttccactggccccctctagcaagtccacctctggaggaacagccgccctgggctgtctggtgaaggattatttcccagagcccgtgacagtgtcttggaacagcggcgccctgacctccggagtgcacacatttccagccgtgctgcagtcctctggcctgtactctctgagctccgtggtgaccgtgccctctagctccctgggcacccagacatatatctgcaacgtgaatcacaagccatctaatacaaaggtggacaagagggtggagcccaagagctgtgataagacccacacatgccccccttgtcctgcaccagagctgctgggaggaccatccgtgttcctgtttccacccaagcctaaggacaccctgatgatctccaggaccccagaggtgacatgcgtggtggtggacgtgtctcacgaggatcctgaggtgaagttcaactggtacgtggatggcgtggaggtgcacaatgccaagacaaagcctcgggaggagcagtacaactctacctatagagtggtgagcgtgctgacagtgctgcaccaggactggctgaacggcaaggagtataagtgtaaggtgagcaataaggccctgcccgcccctatcgagaagaccatctccaaggccaagggccagcctagggagccacaggtgtacaccctgcctccatgccgcgaggagatgacaaagaaccaggtgagcctgtggtgtctggtgaagggcttctatcctagcgacatcgccgtggagtgggagtccaatggccagccagagaacaattacaagaccacaccccctgtgctggactccgatggctctttctttctgtattccaagctgaccgtggataagtctcggtggcagcagggcaacgtgttcagctgctccgtgatgcacgaagcactgcacaaccattacactcagaagtcactgtccctgtctcctggcaagggaggaggaggatccgcggccgcagaggtgcagctgctggagtctggcggcggcctggtgcagcccggcggcagcctgcggctgtcctgtgccgcctctggcttcaccttttctagctacgcaatgagctgggtgagacaggcccctggcaagggcctggagtgggtgtccgccatctccggatctggaggctctacatactatgccgacagcgtgaagggccggttcaccatcagcagagataactccaagaatacactgtatctgcagatgaacagcctgagggccgaggacaccgccgtgtactattgcgccaagatgaagtccacacgccactggatcgacgattggggccagggcaccctggtgacagtgtcctctggaggaggaggatccggaggaggaggctctggcggcggcggcagctccgagctgacccaggaccctgccgtgagcgtggccctgggccagaccgtgaggatcacatgtcagggcgattccctgcgctcttactatgcctcctggtaccagcagaagccaggacaggcacccgtgctggtcatctacggcaagaacaataggccaagcggcatccccgatcgcttttccggctctagctccggcaataccgcaagcctgaccatcacaggagcacaggcagaggacgaggcagattactattgccagtcctacgactcctcctcctaccattacgtctttggcggcggaactaaactgaccgtcctg

SEQ ID NO: 89 - protein, 579 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSAAAEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKMKSTRHWIDDWGQGTLVTVSSGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCQSYDSSSYHYVFGGGTKLTVL

SEQ ID NO: 90 - DNA, 321 bp, synthetic construct
  source 1..321: mol_type = other DNA
cgtacggtcgccgccccctccgtgtttatttttcctccatctgacgaacagctgaagagtgggaccgcctccgtggtgtgcctgctgaacaatttctacccccgggaggccaaggtgcagtggaaagtcgacaacgctctgcagtctggcaatagtcaggagtcagtgactgaacaggacagcaaggattccacctattctctgagctccaccctgacactgagcaaagcagattacgaaaagcacaaagtctatgcctgcgaagtgacccaccaggggctgagcagtccagtgaccaagtcctttaacaggggagagtgt

SEQ ID NO: 91 - protein, 107 aa, synthetic construct [sequence omitted]

SEQ ID NO: 92 - DNA, 1737 bp, synthetic construct
  source 1..1737: mol_type = other DNA
gctagcaccaagggcccatccgtgtttccactggccccctctagcaagtccacctctggaggaacagccgccctgggctgtctggtgaaggattatttcccagagcccgtgacagtgtcttggaacagcggcgccctgacctccggagtgcacacatttccagccgtgctgcagtcctctggcctgtactctctgagctccgtggtgaccgtgccctctagctccctgggcacccagacatatatctgcaacgtgaatcacaagccatctaatacaaaggtggacaagagggtggagcccaagagctgtgataagacccacacatgccccccttgtcctgcaccagagctgctgggaggaccatccgtgttcctgtttccacccaagcctaaggacaccctgatgatctccaggaccccagaggtgacatgcgtggtggtggacgtgtctcacgaggatcctgaggtgaagttcaactggtacgtggatggcgtggaggtgcacaatgccaagacaaagcctcgggaggagcagtacaactctacctatagagtggtgagcgtgctgacagtgctgcaccaggactggctgaacggcaaggagtataagtgtaaggtgagcaataaggccctgcccgcccctatcgagaagaccatctccaaggccaagggccagcctagggagccacaggtgtacaccctgcctccatgccgcgaggagatgacaaagaaccaggtgagcctgtggtgtctggtgaagggcttctatcctagcgacatcgccgtggagtgggagtccaatggccagccagagaacaattacaagaccacaccccctgtgctggactccgatggctctttctttctgtattccaagctgaccgtggataagtctcggtggcagcagggcaacgtgttcagctgctccgtgatgcacgaagcactgcacaaccattacactcagaagtcactgtccctgtctcctggcaagggaggaggaggatccgcggccgcagaagtccaactgctggaatcgggtggtggtctggtgcaaccgggcggctcgctgcgtctgtcatgtgctgcgtcgggctttacctttagcgattatgcaatgtcctgggtgcgtcaggcaccgggtaaaggtctggaatgggttagcgcaattagatggcatagcagctggacctattacgccgattctgttaaaggtcgttttaccatctcacgcgacaactcgaaaaatacgctgtatctgcagatgaacagtctgcgcgcagaagataccgctgtctattactgcgcaaaaatgaagtcgactcggcattggatcgacgattggggccaaggtacgctggttacggttagcagcggcggtggtggcagcggtggtggcggctcgggtggtggcggctcgtctgaactgacgcaagatccggctgtttctgtggcactgggtcaaacggttcgtatcacgtgtcaaggcgattcactgcgctcctattacgcaagttggtatcagcaaaaaccgggccaggctccggtgctggttatttacggcaaaaacaatcgtccgtccggtatcccggatcgcttttcaggcagctctagtggtaacaccgcgtcgctgaccattacgggtgcgcaagccgaagatgaagcagactattactgccagagctatgacagcagcagctaccattatgtctttggtggtggtacgaaactgaccgtcctg

SEQ ID NO: 93 - protein, 579 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSAAAEVQLLESGGGLVQPGGSLRLSCAASGFTFSDYAMSWVRQAPGKGLEWVSAIRWHSSWTYYADSVKGRFTISRDNSKNTLYLQMNSLRAEDTAVYYCAKMKSTRHWIDDWGQGTLVTVSSGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCQSYDSSSYHYVFGGGTKLTVL

SEQ ID NO: 94 - DNA, 990 bp, synthetic construct
  source 1..990: mol_type = other DNA
gctagcactaaaggaccaagcgtgttccctctggctccaagcagcaaatcaacctcaggcggcacagcagcactggggtgtctggtgaaggactacttcccagagcccgtcaccgtgtcatggaacagcggagcactgactagcggagtccacacctttccagccgtgctgcagagctccggactgtactccctgtctagtgtggtcacagtgccttcaagctccctggggactcagacctatatctgcaacgtgaatcacaagccctccaatactaaggtcgacaaacgagtggagcctaagtcttgtgataaaacacatacttgccccccttgtcctgcaccagaactgctgggaggacctagcgtgttcctgtttccacccaagccaaaagacaccctgatgattagtagaacccctgaggtcacatgcgtggtcgtggacgtgagccacgaggatccagaagtcaagttcaactggtacgtggatggcgtcgaagtgcataatgctaagacaaaaccccgggaggaacagtacaacagtacctatagagtcgtgtcagtcctgacagtgctgcatcaggactggctgaacgggaaagagtataagtgcaaagtgtccaataaggccctgcccgctcctatcgagaaaactatttctaaggctaaaggccagccaagggaaccccaggtgtacaccctgcctccatcacgcgaggaaatgacaaagaaccaggtcagcctgacttgtctggtgaaagggttctatccatctgacatcgcagtggagtgggaaagtaatggacagcccgaaaacaattacaagaccacaccccctgtgctggactccgatggatctttctttctgtatagcaagctgaccgtggataaatcccggtggcagcagggcaatgtcttttcttgtagtgtgatgcacgaggcactgcacaaccactacacccagaagtcactgtccctgagccccggaaag

SEQ ID NO: 95 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 96 - DNA, 1362 bp, synthetic construct
  source 1..1362: mol_type = other DNA
gaggtgcagctggtcgagagcggcggcggcctggtgcagcctggaggcagcctgcggctgtcttgttctgccagcggcttcacattcagctccttcggcatgcactgggtcagacagacacccggcaaaggcctggaatgggtggcctacatcagcagcggcagctccaccatctactacggcgacaccgttaagggtagattcaccattagccgggacaacgccaagaactccctgtttctgcagatgagcagcctgagagccgaggatacagccgtgtactactgcgccagagaaggcggatactactacggaagaagctactacacaatggactactggggccagggcacaaccgtgacagtgtccagcgctaagaccacagctcctagcgtgtatcctctcgcccctgtgtgcggcgacaccaccggctctagcgtgaccctgggatgtctggtgaagggctactttcctgagcccgtgacactgacatggaacagcggatctctgtctagcggcgtgcacacctttccagccgtgctgcagagcgatctgtacacactgtctagcagcgtgacagtgaccagcagcacatggcctagccagagcatcacctgtaatgtggcccatcctgcttctagcaccaaggtggacaagaagatcgaacccagaggccccaccatcaaaccttgccctccttgtaaatgccctgcccctaatctgctgggcggaccttctgttttcatcttcccaccaaagatcaaggacgtgctgatgatcagcctgagccccattgtgacctgcgtggtcgtggacgtttccgaggatgaccccgacgtgcagatcagctggttcgtgaacaacgtggaagtgcacacagcccagacacagacccacagagaggactacaacagcaccctcagagtggtgtccgccctgcccatccagcaccaggactggatgagcggcaaagagttcaagtgcaaggtgaacaacaaggatctgcctgctccaatcgagagaacaatctctaagcccaagggctctgttagagcccctcaagtgtgcgtgctgcctcctcctgaggaagagatgaccaaaaagcaggtgaccctgagctgcgccgtgaccgacttcatgcctgaagatatctacgtggaatggaccaacaacggcaagaccgagctgaactacaagaacacagagccagtgctggatagcgacggctcctacttcatggtgtccaagctgcgggtggaaaagaaaaactgggtggaacggaactcttatagctgcagcgtggtccacgagggcctgcacaaccaccacaccaccaagagcttcagcaggacccctggcaag

SEQ ID NO: 97 - protein, 454 aa, synthetic construct
EVQLVESGGGLVQPGGSLRLSCSASGFTFSSFGMHWVRQTPGKGLEWVAYISSGSSTIYYGDTVKGRFTISRDNAKNSLFLQMSSLRAEDTAVYYCAREGGYYYGRSYYTMDYWGQGTTVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVCVLPPPEEEMTKKQVTLSCAVTDFMPEDIYVEWTNNGKTELNYKNTEPVLDSDGSYFMVSKLRVEKKNWVERNSYSCSVVHEGLHNHHTTKSFSRTPGK

SEQ ID NO: 98 - DNA, 2109 bp, synthetic construct
  source 1..2109: mol_type = other DNA
gaggtgcagctggtcgagagcggcggcggcctggtgcagcctggaggcagcctgcggctgtcttgttctgccagcggcttcacattcagctccttcggcatgcactgggtcagacagacacccggcaaaggcctggaatgggtggcctacatcagcagcggcagctccaccatctactacggcgacaccgttaagggtagattcaccattagccgggacaacgccaagaactccctgtttctgcagatgagcagcctgagagccgaggatacagccgtgtactactgcgccagagaaggcggatactactacggaagaagctactacacaatggactactggggccagggcacaaccgtgacagtgtccagcgctaagaccacagctcctagcgtgtatcctctcgcccctgtgtgcggcgacaccaccggctctagcgtgaccctgggatgtctggtgaagggatatttccctgagcccgtgacattgacatggaactccggcagcctgagctccggcgtgcatacctttccagctgtgctgcaatctgatctgtacaccctgtcgagcagcgtgactgtgacatctagcacctggccctctcagagcatcacctgcaacgtggcccaccccgcttctagcaccaaggtggataagaaaatcgagcctagaggccccaccatcaagccctgccccccctgtaaatgccctgcccctaacctgctgggcggacctagcgtgttcatcttccctccaaagatcaaagacgtgctgatgatctctctgagccctatcgtgacctgtgtggtggtcgacgtgagtgaagatgatcctgatgtgcagatcagctggttcgtgaacaatgtggaagtgcatacagcccagacccagacacacagagaggactacaacagcacactgagagtggtgtccgccctgcctattcagcaccaggactggatgagcggaaaggagttcaagtgcaaggtgaacaacaaggacctgcctgctccaatcgagcggaccatcagcaagcctaagggcagcgtgcgggcccctcaagtgtacgtgctgcctccatgtgaagaagagatgaccaagaagcaggtgacactgtggtgcatggtgactgatttcatgcctgaggacatctacgtggaatggaccaacaacggcaagaccgagctgaactacaagaacaccgagcctgtgctggacagcgatggctcctactttatgtacagcaagctgcgggtggaaaagaaaaattgggtcgagagaaactcttatagctgcagcgtggtccacgagggcctgcacaatcaccacaccacaaagtccttcagcagaacacctggcaaaggcggtggtggcagcgcggccgcagaggtgcagctgctggagtctggcggcggcctggtgcagccaggcggcagcctgcggctgtcctgtgccgcctctggcttcaccttttctagctacgccatgagctgggtgagacaggcccctggcaagggcctggagtgggtgtccgccatctccggatctggaggctctacatactatgccgacagcgtgaagggccggttcaccatcagcagagataactccaagaatacactgtatctgcagatgaacagcctgagggccgaggacaccgccgtgtactattgcgccaagatgaagtccacacgccactggatcgacgattggggccagggcaccctggtgacagtgtcctctggaggaggaggatccggaggaggaggctctggcggcggcggcagctccgagctgacccaggaccctgccgtgagcgtggccctgggccagaccgtgaggatcacatgtcagggcgattccctgcgctcttactatgcctcctggtaccagcagaagccaggacaggcacccgtgctggtcatctacggcaagaacaataggccaagcggcatccccgatcgcttttccggctctagctccggcaataccgcaagcctgaccatcacaggagcacaggcagaggacgaggcagattactattgccagtcttatgatagcagctcttaccattatgtcttcggagggggcaccaaactgactgtcctg

SEQ ID NO: 99 - protein, 703 aa, synthetic construct
EVQLVESGGGLVQPGGSLRLSCSASGFTFSSFGMHWVRQTPGKGLEWVAYISSGSSTIYYGDTVKGRFTISRDNAKNSLFLQMSSLRAEDTAVYYCAREGGYYYGRSYYTMDYWGQGTTVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPCEEEMTKKQVTLWCMVTDFMPEDIYVEWTNNGKTELNYKNTEPVLDSDGSYFMYSKLRVEKKNWVERNSYSCSVVHEGLHNHHTTKSFSRTPGKGGGGSAAAEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKMKSTRHWIDDWGQGTLVTVSSGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCQSYDSSSYHYVFGGGTKLTVL

SEQ ID NO: 100 - DNA, 657 bp, synthetic construct
  source 1..657: mol_type = other DNA
gatgtggtgatgacccagtctccactgagcctgcctgctacacctggagatcctgccagcatcagctgcagatcttctcagagcatcgtgcacagcaacggaaatacctacctggaatggtacctgcaaaagcccggccagagccctaaactgctgatctataaggtgtctaaccggttcagcggcgtgcctgatagattcagcggcagcggctccggcacagacttcaccctgaccatcagtagagtggacgccgaggacgtgggcatctactactgttttcagggcagccacgtgccccccaccttcggccctggcacaaagctggaaatcaagcgggccgacgctgcccctaccgtgtctattttccccccttccagcgagcagctgacttccggcggagccagcgtggtctgctttctgaacaacttctaccctaaggacatcaacgtgaaatggaagatcgacggctctgaacggcagaacggcgtgctgaatagctggaccgaccaggactccaaggatagcacctacagcatgagcagcaccctgacactcaccaaggacgagtacgagagacacaacagctacacctgcgaggccacacataagaccagcacatccccaatcgttaagagcttcaacagaaacgagtgc

SEQ ID NO: 101 - protein, 219 aa, synthetic construct
DVVMTQSPLSLPATPGDPASISCRSSQSIVHSNGNTYLEWYLQKPGQSPKLLIYKVSNRFSGVPDRFSGSGSGTDFTLTISRVDAEDVGIYYCFQGSHVPPTFGPGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 102 - DNA, 1362 bp, synthetic construct
  source 1..1362: mol_type = other DNA
gaggtgcagctggtcgagagcggcggcggcctggtgcagcctggaggcagcctgcggctgtcttgttctgccagcggcttcacattcagctccttcggcatgcactgggtcagacagacacccggcaaaggcctggaatgggtggcctacatcagcagcggcagctccaccatctactacggcgacaccgttaagggtagattcaccattagccgggacaacgccaagaactccctgtttctgcagatgagcagcctgagagccgaggatacagccgtgtactactgcgccagagaaggcggatactactacggaagaagctactacacaatggactactggggccagggcacaaccgtgacagtgtccagcgctaagaccacagctcctagcgtgtatcctctcgcccctgtgtgcggcgacaccaccggctctagcgtgaccctgggatgtctggtgaagggatatttccctgagcccgtgacattgacatggaactccggcagcctgagctccggcgtgcatacctttccagctgtgctgcaatctgatctgtacaccctgtcgagcagcgtgactgtgacatctagcacctggccctctcagagcatcacctgcaacgtggcccaccccgcttctagcaccaaggtggataagaaaatcgagcctagaggccctactatcaagccctgccctccttgcaagtgtcctgcccccaacctgctcggcggacctagcgtctttatcttcccccccaagatcaaagacgtgctgatgatctcactgagccctatcgtgacatgcgtggtggtcgacgtgtccgaggacgatcctgatgtgcagatctcctggttcgtgaataatgtggaggtgcacaccgcccagacccagacccaccgggaagattacaacagcaccctgagggtggtgtctgccctgcccatccaacaccaggactggatgagcggcaaggagttcaagtgcaaggtgaacaacaaggacctgcctgcccctattgagcggaccatcagcaagcctaagggcagcgtgcgggcccctcaggtgtacgtgctgcctccaccagaggaagagatgacaaaaaagcaggtgaccctgacctgtatggtgaccgacttcatgcctgaggacatctacgtggaatggaccaacaatggcaaaaccgagctgaactacaagaacaccgagccagtcctggacagcgacggcagctacttcatgtacagcaagctgagagtggaaaagaagaattgggttgaaagaaacagctatagctgcagcgtcgtgcacgagggactgcacaaccaccacaccaccaagagcttcagcagaacacccggcaag

SEQ ID NO: 103 - protein, 454 aa, synthetic construct
EVQLVESGGGLVQPGGSLRLSCSASGFTFSSFGMHWVRQTPGKGLEWVAYISSGSSTIYYGDTVKGRFTISRDNAKNSLFLQMSSLRAEDTAVYYCAREGGYYYGRSYYTMDYWGQGTTVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 104 - DNA, 990 bp, synthetic construct
  source 1..990: mol_type = other DNA
gcctctaccaaaggaccttctgtgtttcctctggcccctagctctaagtccacctctggcggaaccgccgctctgggatgcctggtcaaggactatttccccgaaccagtgaccgtgtcttggaactccggcgccctgacctccggcgtgcacaccttccccgccgtgctgcagtcctctggcctgtactccctgagcagtgtggtcaccgtgccttcctcctccttggggacacagacctacatctgcaacgtgaatcacaagccctccaacaccaaggtggacaagaaagtggaacccaagtcttgcgacaaaacacatacctgccctccttgtcctgctcctgaactgctgggcggcccatctgtgtttctgttcccccctaagcccaaagatacactgatgatctccagaacccctgaagtgacctgtgtggtcgtggacgtgtcccacgaggatcctgaggtgaagttcaactggtacgtggacggcgtggaagtgcacaacgctaagaccaaacctcgggaagagcagtacgccagcacctacagagtggtcagcgtgctgacagtgctgcaccaggactggctgaacggcaaagagtacaagtgcaaggtgtccaacaaggctctgcctgcccctatcgagaagaccatctctaaggccaagggccaacctagagagccccaagtgtgcaccctgcccccatctcgggacgagctgaccaagaaccaggtgtctctgtcttgtgccgtcaagggattctacccttccgacatcgccgtcgagtgggagtctaacggccagcctgagaacaactacaagacaacacctcctgtgctggactccgatggctccttcttcctggtgtccaagctgaccgtggataagagcagatggcagcagggaaatgttttttcctgctccgtgatgcacgaggctctgcataatcactacacccagaagtccctcagcctgtctccaggcaag

SEQ ID NO: 105 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 106 - DNA, 1737 bp, synthetic construct
  source 1..1737: mol_type = other DNA
gccagcaccaagggccctagcgtgttccctctggccccttcgtccaagtctacctccggcggcactgccgccctgggctgtctggtgaaggactacttccctgaacccgtcaccgtgtcctggaactccggcgccctgacctctggcgtgcacacctttcccgccgtgctgcagtcttctggactgtactccctgtcctccgtcgtgaccgtaccttcctcctccctgggaacccagacctacatctgcaacgtgaatcacaagcccagcaataccaaagtggataagaaagtggaacctaagtcctgcgacaaaacccacacatgtccaccctgtcctgctccagagctgctgggcggcccatccgtgttcctgttccctccaaaacccaaggacaccctgatgatctccagaacccccgaggtgacctgcgtggtggtggacgtgtcccacgaggaccctgaggtcaagttcaactggtacgtggacggcgtcgaagtgcataacgccaagaccaagcctcgggaggaacagtacgccagcacctacagagtggtgtccgtgttgacagtgctgcaccaggattggctgaacggcaaagaatacaagtgcaaggtgtccaacaaggccctgcccgcccctatcgagaagaccatctctaaggccaagggccagcctagagagcctcaggtgtacaccctgcccccttgcagagatgagcttaccaagaaccaggtctctctgtggtgcctggtgaaaggcttctacccttccgacatcgccgtagaatgggagtccaatggccagcctgagaacaactacaagaccacccctcctgtgctggactccgacggctctttctttctgtacagtaagctgaccgtggacaagtctagatggcagcagggcaacgtgttcagctgctccgtgatgcacgaagctctgcacaaccattacacacagaagagcctgtctctgtcccctggcaagggcggaggtggctctgccgctgcagaggtgcagctgctggaatctggcggtggcctggttcagcccggcggctccctgagactgtcctgcgctgcttctggcttcaccttctctgactatgccatgtcttgggtgcgccaggctccaggcaagggcctggagtgggtttccgccatccggtggcactcctcctggacctattacgccgactccgtgaagggcagatttacaattagccgggacaactccaagaacaccctgtacctgcagatgaacagcctgagagccgaggataccgctgtgtactactgcgctaagatgaagtccaccagacactggatcgacgactggggccagggcaccctggtgacagtctcctccggaggcggtggatctggtggtggcggatcaggtggaggcgggagctccgagctgacccaagatcctgctgtgtctgtggctctcggccaaactgtgagaatcacctgccaaggcgattccctgcggtcctactacgcctcttggtaccagcagaagcctggacaagctcctgtgctggtgatctacggcaagaacaaccggccctctggcatccctgaccggttcagtggatcctcttccggcaacactgcctctctgaccatcaccggcgctcaggccgaggacgaggccgactactactgtcagagctacgactcctcatcctaccactacgtgttcggcggcggcaccaagctgaccgtcctg

SEQ ID NO: 107 - protein, 579 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSAAAEVQLLESGGGLVQPGGSLRLSCAASGFTFSDYAMSWVRQAPGKGLEWVSAIRWHSSWTYYADSVKGRFTISRDNSKNTLYLQMNSLRAEDTAVYYCAKMKSTRHWIDDWGQGTLVTVSSGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCQSYDSSSYHYVFGGGTKLTVL

SEQ ID NO: 108 - DNA, 1737 bp, synthetic construct
  source 1..1737: mol_type = other DNA
gctagcacaaaagggccttctgtctttccactggctccttcatctaaatctacatctggggggactgccgctctggggtgtctggtcaaggattacttccctgagccagtgaccgtgagctggaactccggcgccctgacctccggagtgcacacatttccagccgtgctgcagagctccggcctgtacagcctgtctagcgtggtgacagtgccttcctctagcctgggcacccagacatatatctgcaacgtgaatcacaagccctccaataccaaggtggacaagaaggtggagcctaagagctgtgataagacccacacatgcccaccttgtccagcacctgaggccgctggaggaccaagcgtgttcctgtttccacccaagcccaaggacaccctgatgatctccaggacccctgaggtgacatgcgtggtggtggacgtgagccacgaggatccagaggtgaagttcaactggtacgtggatggcgtggaggtgcacaatgccaagaccaagccccgggaggagcagtacaactctacctatagagtggtgagcgtgctgacagtgctgcaccaggactggctgaacggcaaggagtataagtgtaaggtgtccaataaggccctgccagcccccatcgagaagaccatcagcaaggcaaagggacagccaagggagcctcaggtgtacaccctgcctccatgccgcgacgagctgacaaagaaccaggtgagcctgtggtgtctggtgaagggcttctatccatctgatatcgccgtggagtgggagagcaatggccagcccgagaacaattacaagaccacaccccctgtgctggactctgatggcagcttctttctgtattccaagctgacagtggacaagtctcgctggcagcagggcaacgtgttttcctgctctgtgatgcacgaggccctgcacaatcactacacccagaagagcctgtccctgtctccaggaaagggaggaggaggatccgcggccgcagaagtccaactgctggaatcgggtggtggtctggtgcaaccgggcggctcgctgcgtctgtcatgtgctgcgtcgggctttacctttagcgattatgcaatgtcctgggtgcgtcaggcaccgggtaaaggtctggaatgggttagcgcaattagatggcatagcagctggacctattacgccgattctgttaaaggtcgttttaccatctcacgcgacaactcgaaaaatacgctgtatctgcagatgaacagtctgcgcgcagaagataccgctgtctattactgcgcaaaaatgaagtcgactcggcattggatcgacgattggggccaaggtacgctggttacggttagcagcggaggaggaggatccggaggaggaggctctggcggcggcggcagctccgagctgacccaggaccctgccgtgagcgtggccctgggccagaccgtgaggatcacatgtcagggcgattccctgcgctcttactatgcctcctggtaccagcagaagccaggacaggcacccgtgctggtcatctacggcaagaacaataggccaagcggcatccccgatcgcttttccggctctagctccggcaataccgcaagcctgaccatcacaggagcacaggcagaggacgaggcagattactattgccagtcctacgactcctcctcctaccattacgtctttggcggcggaactaaactgaccgtcctg

SEQ ID NO: 109 - protein, 579 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSAAAEVQLLESGGGLVQPGGSLRLSCAASGFTFSDYAMSWVRQAPGKGLEWVSAIRWHSSWTYYADSVKGRFTISRDNSKNTLYLQMNSLRAEDTAVYYCAKMKSTRHWIDDWGQGTLVTVSSGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCQSYDSSSYHYVFGGGTKLTVL

SEQ ID NO: 110 - DNA, 2409 bp, synthetic construct
  source 1..2409: mol_type = other DNA
gctagcacaaaagggccttctgtctttccactggctccttcatctaaatctacatctggggggactgccgctctggggtgtctggtcgaggattacttccctgagccagtgaccgtgagctggaactccggcgccctgacctccggagtgcacacatttccagccgtgctgcagagctccggcctgtacagcctgtctagcgtggtgacagtgccttcctctagcctgggcacccagacatatatctgcaacgtgaatcacaagccctccaataccaaggtggacgagaaggtggagcctaagagctgtgataagacccacacatgcccaccttgtccagcacctgagctgctgggaggaccaagcgtgttcctgtttccacccaagcccaaggacaccctgatgatctccaggacccctgaggtgacatgcgtggtggtggacgtgagccacgaggatccagaggtgaagttcaactggtacgtggatggcgtggaggtgcacaatgccaagaccaagccccgggaggagcagtacaactctacctatagagtggtgagcgtgctgacagtgctgcaccaggactggctgaacggcaaggagtataagtgtaaggtgtccaataaggccctgccagcccccatcgagaagaccatcagcaaggcaaagggacagccaagggagcctcaggtgtacaccctgcctccatgccgcgacgagctgacaaagaaccaggtgagcctgtggtgtctggtgaagggcttctatccatctgatatcgccgtggagtgggagagcaatggccagcccgagaacaattacaagaccacaccccctgtgctggactctgatggcagcttctttctgtattccaagctgacagtggacaagtctcgctggcagcagggcaacgtgttttcctgctctgtgatgcacgaggccctgcacaatcactacacccagaagagcctgtccctgtctccaggaaagggaggaggaggatccgcggccgcagaagtccaactgctggaatcgggtggtggtctggtgcaaccgggcggctcgctgcgtctgtcatgtgctgcgtcgggctttacctttagcgattatgcaatgtcctgggtgcgtcaggcaccgggtaaaggtctggaatgggttagcgcaattagatggcatagcagctggacctattacgccgattctgttaaaggtcgttttaccatctcacgcgacaactcgaaaaatacgctgtatctgcagatgaacagtctgcgcgcagaagataccgctgtctattactgcgcaaaaatgaagtcgactcggcattggatcgacgattggggccaaggtacgctggttacggttagcagcgccagcactaaaggaccaagcgtgttccctctggctccaagcagcaaatcaacctcaggcggcacagcagcactggggtgtctggtgaaggactacttcccagagcccgtcaccgtgtcatggaacagcggagcactgactagcggagtccacacctttccagccgtgctgcagagctccggactgtactccctgtctagtgtggtcacagtgccttcaagctccctggggactcagacctatatctgcaacgtgaatcacaagccctccaatactaaggtcgacaaacgagtggagcctaagtcttgtggcggcggcggcagcggaggcggcggctctggcgggggcggctccggcggcggcgggagcgggggaggaggatctggcggcggcggctcttctgaactgacgcaagatccggctgtttctgtggcactgggtcaaacggttcgtatcacgtgtcaaggcgattcactgcgctcctattacgcaagttggtatcagcaaaaaccgggccaggctccggtgctggttatttacggcaaaaacaatcgtccgtccggtatcccggatcgcttttcaggcagctctagtggtaacaccgcgtcgctgaccattacgggtgcgcaagccgaagatgaagcagactattactgccagagctatgacagcagcagctaccattatgtctttggtggtggtacgaaactgaccgtcctggggcagcctaaggccgctccttccgtgacactgttccctccatcctccgaggaactgcaggccaacaaggctaccctcgtgtgcctgatctccgacttttaccctggcgctgtgaccgtggcctggaaggctgatagttctcctgtgaaggccggcgtggaaaccaccacaccttccaagcagtccaacaacaaatacgccgcctcctcctacctgtctctgacccctgaacagtggaagtcccaccggtcctactcttgccaagtgacccatgagggctccaccgtggaaaagaccgtggctcctaccgagtgctct

SEQ ID NO: 111 - protein, 803 aa, synthetic construct
ASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSAAAEVQLLESGGGLVQPGGSLRLSCAASGFTFSDYAMSWVRQAPGKGLEWVSAIRWHSSWTYYADSVKGRFTISRDNSKNTLYLQMNSLRAEDTAVYYCAKMKSTRHWIDDWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGGGGSGGGGSGGGGSGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCQSYDSSSYHYVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 112 - DNA, 321 bp, synthetic construct
  source 1..321: mol_type = other DNA
cgtacggtcgccgccccctccgtgtttatttttcctccatctgacagaaagctgaagagtgggaccgcctccgtggtgtgcctgctgaacaatttctacccccgggaggccaaggtgcagtggaaagtcgacaacgctctgcagtctggcaatagtcaggagtcagtgactgaacaggacagcaaggattccacctattctctgagctccaccctgacactgagcaaagcagattacgaaaagcacaaagtctatgcctgcgaagtgacccaccaggggctgagcagtccagtgaccaagtcctttaacaggggagagtgt

SEQ ID NO: 113 - protein, 107 aa, synthetic construct
RTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 114 - DNA, 990 bp, synthetic construct
  source 1..990: mol_type = other DNA
gctagcacaaagggccctagcgtgttccctctggccccttctagcaagagcaccagcggaggaaccgccgctctgggatgtctggtgaaggactacttccccgagcccgtgacagtgtcttggaacagcggcgccctgacaagcggtgttcataccttccccgctgtgctgcagagcagcggactgtacagcctcagcagcgtggtgaccgtgccttctagctctctgggcacccagacctacatctgcaacgtgaaccacaagcctagtaatacaaaagtggacaagaaggtggaacctaaatcttgcgacaagacccacacatgccctccgtgccccgcccctgaactgctgggcggccccagcgtgtttctgttcccaccaaagcctaaggacaccctgatgatctcccggacccctgaggtgacctgtgtggtcgtggacgtgtcccacgaggatcctgaggttaagttcaactggtatgtggacggcgtggaagtgcacaacgccaaaaccaagcccagagaggaacagtacaacagcacatacagagtggtctccgtgctcacagtgctgcaccaggactggctgaacggcaaggaatacaagtgcaaggtgtccaacaaggccctgcctgctcctatcgagaagaccatcagcaaagccaagggccagcctcgggaaccccaagtgtgcaccctgcctccaagcagagatgagctgaccaagaaccaggtgtctctgagctgcgccgtgaagggcttctacccctccgatatcgccgtggaatgggagagcaatggccaacctgagaacaactacaagacaacccctcctgtgctggacagcgacggcagcttcttcctggtcagcaagctgaccgtggataagtctagatggcagcagggcaacgtgtttagctgtagcgtcatgcacgaggccctgcacaatcactacacacagaaaagcctgtctctgagccccggcaaa

SEQ ID NO: 115 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 116 - DNA, 1737 bp, synthetic construct
  source 1..1737: mol_type = other DNA
gctagcacaaaagggccttctgtctttccactggctccttcatctaaatctacatctggggggactgccgctctggggtgtctggtcaaggattacttccctgagccagtgaccgtgagctggaactccggcgccctgacctccggagtgcacacatttccagccgtgctgcagagctccggcctgtacagcctgtctagcgtggtgacagtgccttcctctagcctgggcacccagacatatatctgcaacgtgaatcacaagccctccaataccaaggtggacaagaaggtggagcctaagagctgtgataagacccacacatgcccaccttgtccagcacctgagctgctgggaggaccaagcgtgttcctgtttccacccaagcccaaggacaccctgatgatctccaggacccctgaggtgacatgcgtggtggtggacgtgagccacgaggatccagaggtgaagttcaactggtacgtggatggcgtggaggtgcacaatgccaagaccaagccccgggaggagcagtacaactctacctatagagtggtgagcgtgctgacagtgctgcaccaggactggctgaacggcaaggagtataagtgtaaggtgtccaataaggccctgccagcccccatcgagaagaccatcagcaaggcaaagggacagccaagggagcctcaggtgtacaccctgcctccatgccgcgacgagctgacaaagaaccaggtgagcctgtggtgtctggtgaagggcttctatccatctgatatcgccgtggagtgggagagcaatggccagcccgagaacaattacaagaccacaccccctgtgctggactctgatggcagcttctttctgtattccaagctgacagtggacaagtctcgctggcagcagggcaacgtgttttcctgctctgtgatgcacgaggccctgcacaatcactacacccagaagagcctgtccctgtctccaggaaagggaggaggaggatccgcggccgcagaagtccaactgctggaatcgggtggtggtctggtgcaaccgggcggctcgctgcgtctgtcatgtgctgcgtcgggctttacctttagcgattatgcaatgtcctgggtgcgtcaggcaccgggtaaaggtctggaatgggttagcgcaattagatggcatagcagctggacctattacgccgattctgttaaaggtcgttttaccatctcacgcgacaactcgaaaaatacgctgtatctgcagatgaacagtctgcgcgcagaagataccgctgtctattactgcgcaaaaatgaagtcgactcggcattggatcgacgattggggccaaggtacgctggttacggttagcagcggaggaggaggatccggaggaggaggctctggcggcggcggcagctccgagctgacccaggaccctgccgtgagcgtggccctgggccagaccgtgaggatcacatgtcagggcgattccctgcgctcttactatgcctcctggtaccagcagaagccaggacaggcacccgtgctggtcatctacggcaagaacaataggccaagcggcatccccgatcgcttttccggctctagctccggcaataccgcaagcctgaccatcacaggagcacaggcagaggacgaggcagattactattgccagtcctacgactcctcctcctaccattacgtctttggcggcggaactaaactgaccgtcctg

SEQ ID NO: 117 - protein, 579 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSAAAEVQLLESGGGLVQPGGSLRLSCAASGFTFSDYAMSWVRQAPGKGLEWVSAIRWHSSWTYYADSVKGRFTISRDNSKNTLYLQMNSLRAEDTAVYYCAKMKSTRHWIDDWGQGTLVTVSSGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCQSYDSSSYHYVFGGGTKLTVL

SEQ ID NO: 118 - DNA, 321 bp, synthetic construct
  source 1..321: mol_type = other DNA
aggaccgtggccgctccttccgtgttcatcttccctccatccgatgagcagctgaagtccggaacagccagcgtcgtgtgcctgctgaacaacttttaccctagagaagccaaggtgcagtggaaggtggacaacgctctccagagcggcaactctcaagagtccgtgaccgagcaggactccaaggactctacatactctctgtcttctaccctgacactgtctaaagccgactacgagaagcacaaggtgtacgcctgcgaggtgacccaccagggcctgtcctctcccgtgaccaagtccttcaacagaggtgaatgc

SEQ ID NO: 119 - protein, 107 aa, synthetic construct [sequence omitted]

SEQ ID NO: 120 - DNA, 990 bp, synthetic construct; identical to SEQ ID NO: 114
  source 1..990: mol_type = other DNA

SEQ ID NO: 121 - protein, 330 aa, synthetic construct; identical to SEQ ID NO: 115

SEQ ID NO: 122 - DNA, 990 bp, synthetic construct
  source 1..990: mol_type = other DNA
gctagcacaaagggccctagcgtgttccctctggccccttctagcaagagcaccagcggaggaaccgccgctctgggatgtctggtggaggactacttccccgagcccgtgacagtgtcttggaacagcggcgccctgacaagcggtgttcataccttccccgctgtgctgcagagcagcggactgtacagcctcagcagcgtggtgaccgtgccttctagctctctgggcacccagacctacatctgcaacgtgaaccacaagcctagtaatacaaaagtggacgagaaggtggaacctaaatcttgcgacaagacccacacatgccctccgtgccccgcccctgaactgctgggcggccccagcgtgtttctgttcccaccaaagcctaaggacaccctgatgatctcccggacccctgaggtgacctgtgtggtcgtggacgtgtcccacgaggatcctgaggttaagttcaactggtatgtggacggcgtggaagtgcacaacgccaaaaccaagcccagagaggaacagtacaacagcacatacagagtggtctccgtgctcacagtgctgcaccaggactggctgaacggcaaggaatacaagtgcaaggtgtccaacaaggccctgcctgctcctatcgagaagaccatcagcaaagccaagggccagcctcgggaaccccaagtgtgcaccctgcctccaagcagagatgagctgaccaagaaccaggtgtctctgagctgcgccgtgaagggcttctacccctccgatatcgccgtggaatgggagagcaatggccaacctgagaacaactacaagacaacccctcctgtgctggacagcgacggcagcttcttcctggtcagcaagctgaccgtggataagtctagatggcagcagggcaacgtgtttagctgtagcgtcatgcacgaggccctgcacaatcactacacacagaaaagcctgtctctgagccccggcaaa

SEQ ID NO: 123 - protein, 330 aa, synthetic construct
ASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 124 - DNA, 990 bp, synthetic construct
  source 1..990: mol_type = other DNA
gcctctaccaaaggcccttccgtgtttcctctggccccttcctccaagtctacatctggcggaacagctgctctgggctgcctggtgaaggactatttccctgaacctgtcacagtgtcttggaactccggcgctctgacaagcggagtccataccttcccggccgtgctgcagtcctccggcctgtattctttgtcctctgtggtaaccgtgccttctagctctctgggcacccagacctacatctgcaacgtgaaccacaagcctagcaacaccaaggtggataagaaggtggaacccaagtcctgcgacaagacccacacctgccccccctgtcctgctcccgaactgctgggcggcccctccgtgtttctgttcccacctaagcctaaggacaccctgatgatctcccggaccccagaggtgacatgcgtggtcgtggacgtgtctcacgaggaccctgaagttaagttcaactggtacgtggatggcgtggaagtgcacaacgccaagaccaagcctagagaagagcagtacgcctccacatacagagtggtgtctgtgctgaccgtgctgcaccaggactggctgaacggcaaagagtacaagtgcaaggtgtccaacaaggccctgcctgctcctatcgagaagaccatctctaaagctaagggccagcccagggagcctcaggtgtacaccctgcctccttcccgggacgagctgaccaagaaccaggtttctctgacctgtctggtgaaaggcttctaccctagcgacatcgccgtggagtgggagtctaatggccagcctgagaacaactacaaaactacccctcctgtgctggacagcgacggcagcttcttcctgtacagcgatctgaccgtcgacaagtccagatggcagcagggcaacgtgttctcttgctccgtgatgcacgaggctctgcataaccactacacccaagagtccctgtctctgtcccccggcaag

SEQ ID NO: 125 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSDLTVDKSRWQQGNVFSCSVMHEALHNHYTQESLSLSPGK

SEQ ID NO: 126 - DNA, 1737 bp, synthetic construct
  source 1..1737: mol_type = other DNA
gcctccaccaagggacctagtgtgttccctctggctccctcctccaagtccacatccggaggcaccgccgccctgggctgcctggtaaaggactacttccccgagcccgtgaccgtgagttggaactccggcgctctgacctctggcgttcatacatttcctgccgtgctgcaatcctctggcctttacagcctgagttctgtggtcaccgtgccttcctcttctttgggaacacagacctacatctgcaacgtgaaccacaagcctagcaacacaaaagtggacaagaaggtggaaccaaagagctgcgacaaaacacacacctgtcctccttgccctgctcctgagctgctgggcggtccctccgtgtttctgttcccacctaagcccaaggacaccctgatgatctctagaacccctgaagttacctgcgtggtcgtggacgtgtctcacgaagatcccgaggtgaagttcaactggtatgtggatggcgtggaagtgcataacgccaaaacaaagcctcgagaagagcagtacgccagcacctacagagtggtgtctgtcttgactgtgctgcaccaggactggctgaacggcaaagagtacaagtgcaaggtgtccaacaaggccttacctgctcctatcgagaagaccatctctaaggccaagggccagcctagagagcctcaggtgtacaccctgcctcctagcagaaaggaactgaccaagaaccaggtgtctctgacctgtctggtgaagggcttctaccccagcgacatcgccgtcgagtgggagtccaacggccagcctgagaacaactacaagaccacccctccagtgctgaagtctgacggctccttcttcctgtactccaagctgaccgttgacaagtcccggtggcagcagggcaatgtgttctcctgctccgtgatgcacgaggccctgcacaaccactacacacagaagtccctgagcctctctccaggcaagggcggaggtggctctgccgccgctgaagtgcagctgctggaaagtggcggtggcctggtgcaacctggcggatctctgagactgtcttgtgccgcttctggctttaccttctccgactacgccatgtcctgggtgaggcaggctcctggaaaaggcctggaatgggtcagcgccatccggtggcactcttcttggacctattacgccgattccgtgaaaggccggttcaccatctcccgggacaactccaagaacaccctgtacctgcagatgaactccctgagagccgaggataccgctgtgtactactgcgccaagatgaaatctaccagacactggatcgacgattggggacaaggcaccctggtcaccgtgtcctctggaggcggtggatctggtggtggcggatcaggtggaggcgggagctccgagctgacccaggatccagcagtgtccgtggctctgggccaaacagtgcggatcacctgtcagggcgactccctgagatcatactacgcttcttggtaccagcagaaacctggccaggctcctgtgctcgtgatctacggcaagaacaatagaccttccggcatccccgaccggttttccggctcctcctctggcaacaccgcttctctgacgatcaccggcgcccaggctgaggacgaggctgattactattgccagtcctacgactctagctcttaccactacgtgttcggcggaggaaccaagctgacagtgctg

SEQ ID NO: 127 - protein, 579 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRKELTKNQVSLTCLVKGFYPSDIAVEWESNGQPENNYKTTPPVLKSDGSFFLYSKLTVDKSRWQQGNVFSCSVMHEALHNHYTQKSLSLSPGKGGGGSAAAEVQLLESGGGLVQPGGSLRLSCAASGFTFSDYAMSWVRQAPGKGLEWVSAIRWHSSWTYYADSVKGRFTISRDNSKNTLYLQMNSLRAEDTAVYYCAKMKSTRHWIDDWGQGTLVTVSSGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCQSYDSSSYHYVFGGGTKLTVL

SEQ ID NO: 128 - DNA, 990 bp, synthetic construct
  source 1..990: mol_type = other DNA
gcctccaccaaaggaccttccgtgtttcctctggccccctcctctaagagcacctctggcggaacagccgctctgggctgcctggttaaggactacttccccgaacctgtgaccgtgtcctggaactccggcgctctgacctctggcgtgcataccttccctgctgtgctgcagtcctctggcctgtactccctgtcctccgtggtgaccgtgccttcttccagcctgggaacccagacctacatttgcaacgtgaaccacaagccctccaacaccaaggtggacaagaaagtggaacccaagtcctgcgacaagacccacacctgtcctccttgccctgctcctgaactgctgggcggccctagtgtgtttctgttcccacctaagcctaaggataccctgatgatctctagaacacccgaggtgacctgcgtggttgtggacgtgtctcacgaggatcctgaagtgaagttcaattggtacgtggatggcgtcgaagtgcacaacgccaaaaccaagcctagagaagagcagtacgcctctacctacagagtggtgtctgtgctgaccgtgctgcaccaggactggctgaacggcaaggagtataagtgcaaggtgagcaacaaggccctgcctgcccctatcgagaagacaatctccaaggccaagggccagcccagagagcctcaggtgtacactctgcctccatctcgcaaagagctgaccaagaaccaggtcagcttgacatgtctggtgaaaggcttctacccctcagacatcgccgtcgagtgggagtccaatggccaacctgagaacaactacaagaccacacctcccgtgctgaagtccgacggctccttcttcctgtactctaagctgacagtggataagtccagatggcagcagggcaacgtgttctcctgctccgtgatgcacgaggccctgcataatcactacacccagaagtccctgtccctgtctcctggcaaa

SEQ ID NO: 129 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRKELTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 130 - DNA, 1737 bp, synthetic construct
  source 1..1737: mol_type = other DNA
gcctctacaaagggcccttctgtgttccctctggctcccagctccaagtctacctctggcggcaccgctgccctgggctgtctggtgaaggattacttcccagagcccgtgaccgtgtcctggaactccggcgctctgacctcaggcgttcatacctttccagccgtgctgcagtctagcggtttgtactccctgtcctccgtggtgaccgtgccttcctcttccctgggcacacagacctacatctgcaacgtgaaccacaagccttccaacaccaaggtggacaagaaggtggaacctaagtcttgcgacaagacccacacctgtcctccttgccctgctccggaactgctgggaggcccttctgtgtttctgttcccccccaaaccaaaggacaccctgatgattagcaggacccctgaagtgacatgcgtggtcgtggacgtgtctcacgaggatcctgaggtgaagttcaactggtacgtggacggcgtggaagtgcacaacgccaaaactaagcccagagaagagcagtacgccagcacctacagagtggtctctgtgctgaccgtgctgcaccaggattggctgaacggcaaagagtacaagtgcaaggtgtctaacaaggctctgcctgcccctatcgagaagaccatctccaaggccaaaggccagcctagagagcctcaggtgtatacactgcctccatctcgggacgagctgacaaagaaccaggtgtccctgacctgcctggtgaagggcttctacccttccgacatcgccgtggagtgggagtccaacggccagcctgagaacaactacaagaccacccctcccgtgctggactccgacggcagcttcttcctgtactccgacctgactgtcgataaatcacggtggcagcagggcaacgtgttctcctgctccgtgatgcacgaagctctgcacaatcattatacccaagagtctctgtccctgtctcctggcaaggggggcggcggctcggctgctgccgaggtgcagctgctggaatccggcggaggactggtccaacctggcggctctctcagactgtcttgtgcggcctccggattcaccttctccgactacgccatgtcctgggtcagacaggctcctggtaagggcctggaatgggtgagcgccatcagatggcactctagctggacctactatgccgattcggtgaaaggccggttcacaatctccagagataactctaagaataccctgtacctgcaaatgaatagtctcagagccgaggacaccgccgtgtactactgcgccaagatgaagtctaccagacactggatcgacgactggggtcagggcaccctggtaaccgtgtcctctggcggaggcggcagtggaggcggcggctccggcggaggcggttccagcgagctgacccaggaccctgctgtgtccgtggccctgggccagaccgtgcggatcacctgtcaaggcgattctctgcggtcttactacgctagttggtaccagcagaagcccggccaggcccctgtgctggtgatctacggcaagaacaatcggccttccggcatccctgaccgattctccggctccagctccggaaacaccgcctccctgaccatcacaggcgcccaggccgaagacgaggccgactactattgccagtcctacgactcctcctcctaccactacgtgttcggcggcggcaccaagctgacagtgctg

SEQ ID NO: 131 - protein, 579 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSDLTVDKSRWQQGNVFSCSVMHEALHNHYTQESLSLSPGKGGGGSAAAEVQLLESGGGLVQPGGSLRLSCAASGFTFSDYAMSWVRQAPGKGLEWVSAIRWHSSWTYYADSVKGRFTISRDNSKNTLYLQMNSLRAEDTAVYYCAKMKSTRHWIDDWGQGTLVTVSSGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCQSYDSSSYHYVFGGGTKLTVL

SEQ ID NO: 132 - DNA, 1737 bp, synthetic construct
  source 1..1737: mol_type = other DNA
gcttctaccaagggcccttcggtatttcctctggccccctcctccaagtctacctctggcggcacggccgccctgggctgcctggtcaaggattacttcccagagcctgtcaccgtttcctggaactccggcgctctgacctccggagtgcacaccttccccgccgtgctgcagtcttctggcctgtactcactgtcctccgtcgtgaccgtgccttcctcttctctgggcacacagacctacatctgcaatgtgaaccacaagcctagcaacaccaaagtggacaagaaggtggaacccaagtcctgcgacaaaacccatacttgtcctccttgccctgcccccgagctgctaggcggcccctccgtgtttctgttccctcctaagcccaaggacaccctgatgatcagccggacccctgaggtgacatgcgtggtggtggatgtgtcccatgaggaccctgaggtgaagttcaactggtatgtggatggcgtggaagtgcacaacgccaagaccaagcctagagaggaacagtacgcttccacctacagagtggtctccgtcctgaccgttctgcaccaggattggctgaacggcaaagagtacaagtgcaaggtgtccaacaaggccctgcctgctcctatcgagaagaccatctccaaagctaagggacagcctcgggagcctcaggtgtacacactgcctccatctagggacgaactgaccaagaaccaggtcagcctgacctgcctggtgaagggcttctacccctccgacatcgccgtggaatgggagtccaacggccaaccagaaaacaactacaaaaccacccctcctgtgctggactctgacggctccttcttcctgtactccgacctgaccgtggacaagtccagatggcagcagggcaatgtgttctcctgctccgtgatgcacgaggctctgcacaaccactacacccaagagagcctgtcgttgtctcctggcaaaggcggcggcggctctgccgctgccgaagtgcagctgctggaatctggcggcggcctggtgcagcctggcggttccctgagactgtcttgtgccgctagcggctttaccttctccgactacgccatgtcctgggtgagacaggctcctggaaagggactcgaatgggtctcggccatcagatggcactccggctggacctactatgctgattctgtgaaaggccgcttcaccatctcccgggataatagcaagaataccttgtacctgcagatgaactccctgcgggctgaggatacagccgtgtactactgcgccaagatgaagtcgaccagacactggatcgacgactggggccagggtacactggtgaccgtgtcctccggaggtggcggatctggagggggcggctctggcggcggaggctcctccgagctgacccaggaccccgccgtgtctgtggctcttggccagaccgtgcggatcacttgccagggcgactccctgagatcctactacgcctcatggtaccagcagaagccaggccaagctcctgtgctggtgatctacggcaagaacaaccggccttctggcatccctgacagattcagcggctcttcatctggcaacacagcttctctgaccatcaccggcgcccaggccgaggacgaggccgattactactgtcagtcctacgactccagctcttaccactacgtgttcggcggcggtacaaagctgacagtgctg

SEQ ID NO: 133 - protein, 579 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSDLTVDKSRWQQGNVFSCSVMHEALHNHYTQESLSLSPGKGGGGSAAAEVQLLESGGGLVQPGGSLRLSCAASGFTFSDYAMSWVRQAPGKGLEWVSAIRWHSGWTYYADSVKGRFTISRDNSKNTLYLQMNSLRAEDTAVYYCAKMKSTRHWIDDWGQGTLVTVSSGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCQSYDSSSYHYVFGGGTKLTVL

SEQ ID NO: 134 - DNA, 1737 bp, synthetic construct
  source 1..1737: mol_type = other DNA
gcttccaccaagggccctagcgtgtttcctctggcccctagctccaagagtacctccggcggcaccgcagctctgggctgcctcgtgaaggactacttccctgaacccgtgacagtgtcttggaactcgggcgctctgaccagtggagtgcacacctttccagccgtgctgcagtcttccggcctgtactccctgagctctgtggtgacagtgccttctagctctctgggcacacagacctacatctgcaacgtgaaccacaaaccctccaacacgaaagtggacaagaaggtggaacctaagtcctgcgacaagacccatacctgccctccttgccctgctcctgagctgctgggcgggccttccgtgtttctgttcccccctaaacccaaggacaccctaatgatctccagaacccctgaagttacatgcgtggtcgtggacgtgtcccacgaggatccagaggtgaagttcaactggtatgtggatggcgtggaagtgcacaacgccaagaccaagccaagagaagagcagtacgcgtccacctacagagtggtgtccgtgctcaccgtgctgcaccaggactggctgaacggcaaagagtacaagtgcaaggtgtccaacaaggccctgccagctcctatcgagaagaccatctctaaggctaagggccaacctagagagcctcaggtgtacaccctgcctccctctcgggacgagctgacaaagaaccaggtctccctgacctgcctggtcaaaggcttctacccctccgacatcgccgtcgagtgggagtccaatggccagcccgagaacaactacaagacaacacctcctgtgctggactccgacggctccttcttcctgtactccgacctgaccgtggataaatctcggtggcagcagggcaacgtgttctcctgctccgtgatgcacgaggctctgcataatcactacacccaagagtctctgagcctgtctcctggaaagggcggaggcggatccgccgctgctgaagtgcagctgctggagtctggcggaggcctggtgcagcctgggggctcactgcggctgtcttgcgccgctagcggctttaccttcagcgattacgccatgtcctgggtgcggcaggccccaggcaagtgcctggaatgggtgtctgccatcagatggcactcctcctggacctactacgctgattctgtgaaaggccggttcaccatttcccgggataatagcaagaataccctgtacctgcagatgaactctctgcgggctgaggacaccgccgtgtactactgcgccaagatgaagtctaccagacactggatcgacgactggggccaaggcaccctggtcaccgtgtcctccggcggcggaggatccggtggcggcgggtctggcggaggcggctcttccgaactgacccaggatcctgccgtgtccgtggccctgggacagacagtacggatcacctgtcagggcgactccctgcgctcttactacgcctcttggtaccagcagaagcctggccaggcccctgtgctggtgatctatggcaagaacaacagaccttctggcatccctgacagattctccggaagctccagcggcaacaccgcttctctcaccatcaccggcgctcaggccgaagatgaggccgactactactgccagtcctacgactcttcctcctaccactacgtgttcggctgtggcaccaagctgaccgtgctg

SEQ ID NO: 135 - protein, 579 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSDLTVDKSRWQQGNVFSCSVMHEALHNHYTQESLSLSPGKGGGGSAAAEVQLLESGGGLVQPGGSLRLSCAASGFTFSDYAMSWVRQAPGKCLEWVSAIRWHSSWTYYADSVKGRFTISRDNSKNTLYLQMNSLRAEDTAVYYCAKMKSTRHWIDDWGQGTLVTVSSGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCQSYDSSSYHYVFGCGTKLTVL

SEQ ID NO: 136 - DNA, 1737 bp, synthetic construct
  source 1..1737: mol_type = other DNA
gcctctacaaagggcccatccgttttccctctggccccttcctctaaatccacctctggcggaaccgctgctctgggctgcctggtgaaggactacttccccgaacccgtgaccgtgtcctggaactccggcgcactgacgtccggcgtgcacacctttcctgccgttctgcaatcttccggcctgtactccctgagctccgtggtcaccgtgccttcttcgtccctgggcacccagacctacatctgcaacgtgaaccacaagcctagtaataccaaggtggacaagaaagtggaaccaaagtcctgcgacaaaacccacacctgtcctccttgccctgctccagaactgctggggggaccttccgtgtttctgttccctccaaagcccaaggatacactgatgatctccagaacccctgaggtgacctgcgtggtcgtggacgtgagtcacgaggatcctgaggtgaagttcaactggtatgtggatggtgtggaagtgcacaacgccaagaccaagcctagagaagagcagtacgcttccacctacagagtggtctctgtgctgaccgtgttgcaccaggattggctgaacggcaaagagtacaagtgcaaggtgtccaacaaggccctgcctgctcccatcgagaagaccatttctaaggccaagggtcagccaagagagcctcaagtgtacaccctgcccccctccagagatgagctgacaaaaaaccaggtgagcctgacctgtctggtgaagggcttctacccctccgacatcgccgtggaatgggagtctaatggccagcccgagaacaactacaagaccacccctcctgtgctggactccgacggctccttcttcctgtactctgacctgaccgtggacaagagcagatggcagcagggcaacgtgttctcctgctccgtgatgcacgaggctctgcataatcattatacccaagagtccctgtctctgtcccccggcaagggaggcggcggatctgctgccgccgaagtgcagctgctggaatccggcggcggactggttcagcctggcggctctctgaggctttcttgcgccgctagcggctttaccttcagcgactacgccatgtcctgggtgcgccaggcccctggcaagtgcctggagtgggtctctgccatcagatggcactctggctggacctactacgctgattctgtgaaaggcagattcaccatctctcgggacaactccaagaacactctgtacctgcagatgaactctctccgggccgaggacactgccgtgtactactgtgcaaagatgaagagcacacggcactggatcgacgactggggccagggcacactggtgacagtgtcttccggaggcggcggatctggaggaggcggctctggcggcggcggaagttccgagctgacacaggaccctgctgtgtcagtggccctcggccagaccgtgcggatcacctgccagggcgactctctgcggtcttactacgcctcttggtaccagcagaagcctggccaggcccctgtgctggtgatctacggcaagaacaaccggccttctggcatccctgacagattcagcggctcctcctctggtaatacggcttccctcaccatcaccggcgctcaggccgaggacgaggccgactactactgccagtcctacgacagctcctcttaccactacgtgttcggctgtggcaccaagctgaccgtgctg

SEQ ID NO: 137 - protein, 579 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSDLTVDKSRWQQGNVFSCSVMHEALHNHYTQESLSLSPGKGGGGSAAAEVQLLESGGGLVQPGGSLRLSCAASGFTFSDYAMSWVRQAPGKCLEWVSAIRWHSGWTYYADSVKGRFTISRDNSKNTLYLQMNSLRAEDTAVYYCAKMKSTRHWIDDWGQGTLVTVSSGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCQSYDSSSYHYVFGCGTKLTVL

SEQ ID NO: 138 - DNA, 1740 bp, synthetic construct
  source 1..1740: mol_type = other DNA
gcctctacaaagggcccttctgtgttccctctggctcccagctccaagtctacctctggcggcaccgctgccctgggctgtctggtgaaggattacttcccagagcccgtgaccgtgtcctggaactccggcgctctgacctcaggcgttcatacctttccagccgtgctgcagtctagcggtttgtactccctgtcctccgtggtgaccgtgccttcctcttccctgggcacacagacctacatctgcaacgtgaaccacaagccttccaacaccaaggtggacaagaaggtggaacctaagtcttgcgacaagacccacacctgtcctccttgccctgctccggaactgctgggaggcccttctgtgtttctgttcccccccaaaccaaaggacaccctgatgattagcaggacccctgaagtgacatgcgtggtcgtggacgtgtctcacgaggatcctgaggtgaagttcaactggtacgtggacggcgtggaagtgcacaacgccaaaactaagcccagagaagagcagtacgccagcacctacagagtggtctctgtgctgaccgtgctgcaccaggattggctgaacggcaaagagtacaagtgcaaggtgtctaacaaggctctgcctgcccctatcgagaagaccatctccaaggccaaaggccagcctagagagcctcaggtgtatacactgcctccatctcgggacgagctgacaaagaaccaggtgtccctgacctgcctggtgaagggcttctacccttccgacatcgccgtggagtgggagtccaacggccagcctgagaacaactacaagaccacccctcccgtgctggactccgacggcagcttcttcctgtactccgacctgactgtcgataaatcacggtggcagcagggcaacgtgttctcctgctccgtgatgcacgaagctctgcacaatcattatacccaagagtctctgtccctgtctcctggcaaggggggcggcggctcggctgctgcctccagcgagctgacccaggaccctgctgtgtccgtggccctgggccagaccgtgcggatcacctgtcaaggcgattctctgcggtcttactacgctagttggtaccagcagaagcccggccaggcccctgtgctggtgatctacggcaagaacaatcggccttccggcatccctgaccgattctccggctccagctccggaaacaccgcctccctgaccatcacaggcgcccaggccgaagacgaggccgactactattgccagtcctacgactcctcctcctaccactacgtgttcggcggcggcaccaagctgacagtgctgggcggaggcggcagtggaggcggcggctccggcggaggcggttccgaggtgcagctgctggaatccggcggaggactggtccaacctggcggctctctcagactgtcttgtgcggcctccggattcaccttctccgactacgccatgtcctgggtcagacaggctcctggtaagggcctggaatgggtgagcgccatcagatggcactctagctggacctactatgccgattcggtgaaaggccggttcacaatctccagagataactctaagaataccctgtacctgcaaatgaatagtctcagagccgaggacaccgccgtgtactactgcgccaagatgaagtctaccagacactggatcgacgactggggtcagggcaccctggtaaccgtgtcctct

SEQ ID NO: 139 - protein, 580 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSDLTVDKSRWQQGNVFSCSVMHEALHNHYTQESLSLSPGKGGGGSAAASSELTQDPAVSVALGQTVRITCQGDSLRSYYASWYQQKPGQAPVLVIYGKNNRPSGIPDRFSGSSSGNTASLTITGAQAEDEADYYCQSYDSSSYHYVFGGGTKLTVLGGGGSGGGGSGGGGSEVQLLESGGGLVQPGGSLRLSCAASGFTFSDYAMSWVRQAPGKGLEWVSAIRWHSSWTYYADSVKGRFTISRDNSKNTLYLQMNSLRAEDTAVYYCAKMKSTRHWIDDWGQGTLVTVSS

SEQ ID NO: 140 - DNA, 1740 bp, synthetic construct
  source 1..1740: mol_type = other DNA
gcttccaccaagggcccctctgtgttccctctggctccatcttctaagtccacctccggcggcacagccgccctgggttgtctggtgaaggattacttccccgagcctgtgacagtgtcctggaactctggagctctgacatctggcgtgcacacctttcctgccgttctgcagtcctccgggctgtactctctgtcctccgtggtcaccgtgccttcctcctccctgggcacacagacctacatctgcaacgtgaaccacaagcctagcaacaccaaggtggacaagaaggtggaacctaagtcctgcgacaagacccacacctgccctccttgtcctgctcccgagctgctgggcggccctagtgtgtttctgttccctcctaaacccaaggacaccctgatgatctcccggacccccgaagtgacctgcgtggtggtggacgtgtcccacgaggacccagaagtgaagttcaattggtatgtggacggcgtcgaggtccacaacgccaagacaaaacctagagaagagcagtacgccagcacctacagagtggtctctgtgctgaccgtgctgcatcaggattggctgaacggcaaagagtacaagtgcaaggtgtctaacaaggccctgcccgctcctatcgagaagaccatctctaaggctaagggccaacctagagagcctcaggtgtacaccctgcctccttctcgggacgaactcacaaagaaccaggtgtccctgacctgcctggtgaagggcttctacccctccgacatcgccgtggaatgggagtccaacggccaacctgagaacaactacaagaccacccctcccgtgctggacagcgatggctccttcttcctgtactccgacctgacagtggataagtccagatggcagcagggcaacgtgttctcctgctccgtgatgcacgaggctctgcataatcactacacccaagagtctctgtctctgagccctggcaaaggcggaggtggctctgccgctgcttcttctgagctgacccaggacccagccgtgtccgtggccctgggccagaccgtgcggatcacctgtcagggcgactccctcagatcctactatgcttcttggtaccagcagaagcctggccaggcccctgtgctggtgatctacggcaagaacaaccggccttccggcatccctgacagattttctggatctagctcaggcaacaccgcctctctgacgatcaccggcgctcaggccgaggacgaggccgattactactgccagtcgtacgactccagcagctaccactacgtgttcggctgcggcaccaagctgaccgtgctgggaggcggtggatctggtggtggcggatcaggtggaggcgggagcgaggtgcagctcctggaatccggcggaggactggtacagcctggaggttccctgagactgagctgtgctgcctctggatttacattctctgactacgccatgtcttgggtcagacaggcaccaggcaagtgcctggaatgggtgtccgccatcagatggcactccagctggacctactacgccgactctgtgaaaggccggttcaccatctcccgcgacaactccaagaacaccctgtacctgcagatgaactccctgagagccgaagacaccgctgtgtactactgcgccaagatgaaatctaccagacactggatcgacgactggggacagggcacactggtgaccgtcagttct

SEQ ID NO: 141 - protein, 580 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSDLTVDKSRWQQGNVFSCSVMHEALHNHYTQESLSLSPGKGGGGSAAASSELTQDPAVSVALGQTVRITCQGDSLRSYYASWYQQKPGQAPVLVIYGKNNRPSGIPDRFSGSSSGNTASLTITGAQAEDEADYYCQSYDSSSYHYVFGCGTKLTVLGGGGSGGGGSGGGGSEVQLLESGGGLVQPGGSLRLSCAASGFTFSDYAMSWVRQAPGKCLEWVSAIRWHSSWTYYADSVKGRFTISRDNSKNTLYLQMNSLRAEDTAVYYCAKMKSTRHWIDDWGQGTLVTVSS

SEQ ID NO: 142 - DNA, 1371 bp, synthetic construct
  source 1..1371: mol_type = other DNA
gcttctacaaaaggcccttccgtctttcctctggctccttcctccaagtccacctcaggcggtaccgccgctctgggctgcctggtcaaggactacttcccggagcccgtgaccgtgtcctggaactccggcgccctcacttccggggttcacaccttccctgccgtgctgcagtcctctggcctgtactctctgagctctgtggtgaccgtgccctcttcttctctcggcacccagacctacatctgcaacgtgaaccacaagccttccaacaccaaagtggataagaaagtggaacctaaatcctgcgacaagacacatacatgtcctccttgccctgcccccgaactgctgggcggcccttctgtgttcctgttcccaccaaagcctaaggacaccctgatgattagcagaacacctgaggttacctgcgtggtggtggacgtgtctcatgaggaccctgaagtgaagttcaactggtacgtggacggcgtcgaggtgcacaacgctaagaccaagccccgggaagagcagtacgcttctacttacagagtggtctccgtgctgaccgtgctgcaccaggattggctgaacggcaaagagtacaagtgcaaggtgtccaacaaggctctgcctgctcctatcgagaagaccatctccaaggccaagggccagcctagagagcctcaggtgtataccttgcccccttctcgggacgagctgaccaagaatcaagtgtctctgacctgtctggtgaagggcttctaccctagtgacatcgccgtggaatgggagtcgaatggccagcccgagaacaactacaagaccacccctcctgtgctggacagcgatggctccttcttcctgtactccgacctgacagtagacaagtctagatggcagcagggcaacgtgttcagctgctccgtgatgcacgaggctctgcacaaccactacacccaagagtctctgtccctgtctcctggaaaaggcggcggcggcagcgccgccgccgaagtgcagctgctggaatccggcggcggactggtgcagcccggaggaagcctgagactgtcctgtgccgccagcggctttaccttttccgattacgccatgtcctgggtgcggcaggcccctggcaagtgcctggaatgggtgtccgccatccggtggcactcgtcttggacctactacgctgactccgtgaagggtcggttcaccatctctcgggacaatagcaagaacacactgtacctgcagatgaactccctccgcgccgaggataccgccgtgtactactgtgccaagatgaagtccaccagacactggatcgacgactggggccaaggaaccctggtgaccgtgtcctcc

SEQ ID NO: 143 - protein, 457 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSDLTVDKSRWQQGNVFSCSVMHEALHNHYTQESLSLSPGKGGGGSAAAEVQLLESGGGLVQPGGSLRLSCAASGFTFSDYAMSWVRQAPGKCLEWVSAIRWHSSWTYYADSVKGRFTISRDNSKNTLYLQMNSLRAEDTAVYYCAKMKSTRHWIDDWGQGTLVTVSS

SEQ ID NO: 144 - DNA, 1338 bp, synthetic construct
  source 1..1338: mol_type = other DNA
gccagtaccaaaggccctagcgtttttcctctggcccctagcagcaagtccaccagcggcggcacagccgcactgggctgcctggtgaaggactacttccctgagcctgtgaccgtgtcctggaactctggcgctctgacctccggagtgcacaccttcccagctgtgctgcagtcctccggcctgtactccctgtccagcgtggtgaccgtgccttcttcatcactgggcacccagacctacatctgcaacgtgaaccacaagccctctaataccaaagtggacaagaaggtggaacccaagtcctgcgacaagacccacacatgccctccttgccccgctcccgaactgctgggcggaccttccgtgttcctgttcccccccaagccaaaagatacactgatgatttcccggacccctgaagtgacctgcgtggtcgtggatgtgagtcatgaggaccctgaggtgaagttcaactggtatgtagatggcgtggaagtgcacaacgccaagaccaagccacgggaagagcagtacgcttctacctacagagtggtgtctgtgttgaccgtgctgcaccaggactggctgaacggcaaagagtacaagtgcaaggtgtctaacaaggctctgcctgcccccatcgagaagaccatctccaaggccaaaggacagcctagagagcctcaagtgtacaccctgcctccttcccgggatgagctgacaaagaaccaggtgtctctgacctgcctggtgaagggcttctatccttccgacatcgccgtcgagtgggagtccaatggccagccagagaacaactacaagaccacccctcccgtgctggactctgacggctccttcttcctgtattccgacctgacagttgataagtccagatggcagcagggcaacgtgttctcctgctccgtgatgcacgaagccctgcataaccactacacccaagagagcctgtctctgtctcctggcaagggcggcggcgggtccgctgctgcttcttctgagctgacccaagatcctgccgtgtccgtggccctgggtcagaccgtgcggatcacctgtcagggcgacagcctgagatcttactacgcctcttggtaccagcagaagcctggccaggcccccgtgctcgtgatctacggcaagaacaaccgcccttctggcatccctgaccggttctctgggtcttcctctggaaatacagctagcctgactatcaccggcgctcaggccgaagacgaggccgactactactgccagtcctacgactccagctcctaccactacgtgtttggctgtggcaccaagctgacagtgctc

SEQ ID NO: 145 - protein, 446 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSDLTVDKSRWQQGNVFSCSVMHEALHNHYTQESLSLSPGKGGGGSAAASSELTQDPAVSVALGQTVRITCQGDSLRSYYASWYQQKPGQAPVLVIYGKNNRPSGIPDRFSGSSSGNTASLTITGAQAEDEADYYCQSYDSSSYHYVFGCGTKLTVL

SEQ ID NO: 146 - DNA, 324 bp, synthetic construct
  source 1..324: mol_type = other DNA
agtagtgaactaactcaagatccagcagtgtccgtggccctgggccagaccgtgcggatcacctgtcagggcgactctctgagatcctattacgcctcttggtaccagcagaaacctggacaagctcctgtgctggtgatctacggcaagaacaaccggccttctggcatccccgatagattctccggatcttctagcggcaacaccgcttccctgaccatcaccggcgctcaggccgaggacgaagccgactactactgccagtcctacgactcctccagctaccactacgtgttcggctgcggcaccaagctgacagtcctg

SEQ ID NO: 147 - protein, 108 aa, synthetic construct
SSELTQDPAVSVALGQTVRITCQGDSLRSYYASWYQQKPGQAPVLVIYGKNNRPSGIPDRFSGSSSGNTASLTITGAQAEDEADYYCQSYDSSSYHYVFGCGTKLTVL

SEQ ID NO: 148 - DNA, 1737 bp, synthetic construct
  source 1..1737: mol_type = other DNA
gcctctacaaaaggccccagcgtctttcctctcgccccatcctctaagtctacctctggtggcaccgctgctctgggctgcctggtcaaggactacttcccagagcccgtgacagtctcatggaacagcggcgctctgacctctggagtgcataccttcccagctgtgctgcagtcctctggcctgtactccctgtctagcgtggtgaccgtgccttccagctctctgggcacccagacctacatctgcaacgtgaaccacaagccatccaacaccaaagtggacaagaaggtggaacccaagtcttgcgacaagacccacacctgtcctccttgccctgccccagagctgctggggggcccttctgtgttcctgttcccccccaagcctaaggataccctgatgatctccagaacccctgaagtgacctgcgtggtggtcgacgtcagccacgaggatcctgaggtgaagttcaactggtacgtggatggcgttgaagtgcacaacgccaaaaccaagcctagagaagagcagtacgcctccacctacagagtggtgtccgtgctgaccgtgctgcaccaggactggctgaacggcaaagagtacaagtgcaaggtctccaacaaggccctgcctgctcctatcgagaagaccatctctaaggcgaagggccagcctagagagcctcaggtgtataccctgcccccttcgcgggacgagctgacaaagaaccaggtgtctctgacctgcctggtaaagggcttctacccctctgacatcgccgtggaatgggagtctaacggccagcctgagaacaactacaagaccacccctcctgtgctggactccgacggctccttcttcctgtacagcgacctgaccgtggacaagtctcggtggcagcagggcaatgtgttctcctgctccgtgatgcacgaggccctgcataatcactacacccaagagtccctgtccctttctcctggcaagggcggaggtggctctgccgctgcagaagtgcagctggtggaatctggaggcggcctggtgcagcccggtggcagcctgagactgtcttgtgctgcctctggcttcacctttagcgactacgccatgtcctgggtgcggcaggcccctggaaagggcctcgagtgggtgtccgccattagatggcactcttcttggacctactacgccgactctgtgaaaggccggttcacaatctcccgggacaactccaagaacaccctgtacctgcagatgaactccctgagagctgaggacacggctgtatactactgtgccaagatgaaatctaccagacactggatcgacgattggggccagggcacattggtcacagtgtcctctggaggcggtggatctggtggtggcggatcaggtggaggcgggagctccgagctgacccaggatcctgccgtgtccgtggccttgggacagaccgtgcggatcacctgtcagggcgacagcctgagaagctattatgccagctggtaccagcagaagcccggccaggccccggttctggtgatctacggcaagaacaatcgcccttccggcatccctgaccggttctccggatcttcctccggtaacaccgcctccctgaccatcaccggcgcccaagctgaggatgaggctgactactactgccagtcttacgattcttccagttaccactacgtgttcggcggcggcaccaagctgaccgtgctg

SEQ ID NO: 149 - protein, 579 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSDLTVDKSRWQQGNVFSCSVMHEALHNHYTQESLSLSPGKGGGGSAAAEVQLVESGGGLVQPGGSLRLSCAASGFTFSDYAMSWVRQAPGKGLEWVSAIRWHSSWTYYADSVKGRFTISRDNSKNTLYLQMNSLRAEDTAVYYCAKMKSTRHWIDDWGQGTLVTVSSGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCQSYDSSSYHYVFGGGTKLTVL

SEQ ID NO: 150 - DNA, 1737 bp, synthetic construct
  source 1..1737: mol_type = other DNA
gccagtaccaagggaccttctgtgtttcctctggccccctcaagcaagtctacgtccggcggaacagctgcactgggctgcctggtgaaggactacttcccagagcccgtgaccgtgtcttggaacagcggcgccctgacctctggcgtgcatacattccctgccgtgctgcagtctagcggcctgtacagcctgtcctccgtcgtgaccgtgccttccagctctctgggcacccagacctacatctgcaacgtgaaccacaagcccagcaacaccaaagtggacaagaaggtggaacctaagtcctgcgacaagacccacacctgtcctccttgccctgcaccggaactgctgggcggccctagcgtgttcctgttcccccccaagcctaaggacactctgatgatctctagaacccctgaggtgacctgcgtggtggtggacgtgtctcacgaggatcctgaagtgaagttcaactggtatgtggatggcgtcgaagtgcacaacgccaagaccaagcctcgggaagagcagtacgcttccacctacagagtggtcagcgtgctgaccgtgctgcaccaggattggctgaacggcaaagagtacaagtgcaaggtgtctaacaaggctctgcctgcccctatcgagaagacaatttctaaggccaaaggacagcctagggagcctcaggtgtacaccctgcctccttccagagatgagctgacaaagaaccaggtgtcccttacctgcctggtgaagggcttctacccctccgacatcgccgtggaatgggaatccaacggccagcctgagaacaactacaagaccacacctccagtgctggattccgacggctccttcttcctgtactctgacctgaccgtggacaaaagccggtggcagcagggcaacgtgttctcttgctccgtgatgcacgaggctctgcataatcactatactcaagagtctctgtctctgtcccctggcaagggaggcggcggatctgctgctgctgaggtgcagctgcaagagtctggaggcggactcgtgcagccaggcggctccctgcggctgtcctgtgctgcctctggcttcacgttctccgactacgccatgtcctgggtgcggcaggctcctggcaaaggcctggaatgggtctccgccatcagatggcactcctcctggacctactacgccgattctgtgaaaggcagatttaccatctcccgggacaactctaagaacaccctgtacctgcagatgaactctttgcgcgccgaggacaccgctgtttattactgcgccaagatgaagtctaccagacactggatcgacgactggggccaaggcaccctcgtcaccgtatcctctggaggcggcggctctggcggcggcggttctggcggaggcggctcctccgagctgacccaggaccccgctgtgtccgtggccctgggacaaaccgtgcggatcacctgtcaaggcgattccctgcggtcctactatgctagttggtaccagcagaagcccggccaggctcctgtgctcgtgatctacggcaagaataatcggccttctggcatccctgacagattctctggctctagctccggcaataccgcttctctgacaatcaccggcgcccaggccgaggacgaggccgactactactgccagagctacgactcttcctcctaccactacgtgttcggcggcggcaccaaactgacagtgctg

SEQ ID NO: 151 - protein, 579 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSDLTVDKSRWQQGNVFSCSVMHEALHNHYTQESLSLSPGKGGGGSAAAEVQLQESGGGLVQPGGSLRLSCAASGFTFSDYAMSWVRQAPGKGLEWVSAIRWHSSWTYYADSVKGRFTISRDNSKNTLYLQMNSLRAEDTAVYYCAKMKSTRHWIDDWGQGTLVTVSSGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCQSYDSSSYHYVFGGGTKLTVL

SEQ ID NO: 152 - DNA, 1737 bp, synthetic construct
  source 1..1737: mol_type = other DNA
gcctccaccaagggcccttccgtgtttcctctggctccttctagcaagtccacctccggcggcaccgccgctctcggctgcctggtgaaggactacttccccgagcccgtgaccgtgtcttggaactccggcgctctgacttctggcgtgcacaccttccctgccgtgctgcagtcttctggcctgtactctctgagcagcgtggtgaccgtgccctccagctctctgggcacccagacctacatctgcaacgtgaaccacaagccgtcgaataccaaagtggacaagaaggtggaacccaaatcctgcgacaagacccacacatgtcctccttgccctgctcctgaactgctggggggcccctccgtgttcctgttcccacctaagcccaaggataccctgatgatctcccggacccccgaagtcacctgcgtggtagtcgacgtgtctcatgaggatccagaggtcaagttcaactggtatgtggacggcgtggaagtccataacgcgaagaccaagcctagagaagagcagtacgcttctacctatagagtggtctctgtgctgacagtgctgcaccaggactggctgaacggcaaagagtacaagtgcaaggtgtccaacaaggctctgcctgctcccatcgagaagaccatctccaaggccaagggccagcctagagagcctcaggtctataccctgcctccttccagagatgagctgacaaaaaaccaggtgagtctgacctgcctggtgaagggcttctaccctagcgacatcgccgttgagtgggagtccaacgggcaacctgagaacaactacaagacaacccctcctgtgctggactctgacggatccttcttcctgtactccgacctgaccgtggataagtcccggtggcagcagggaaatgtgttctcctgctccgtgatgcacgaggccctgcacaatcactacacccaagagtctctgtccctgtctcctggcaaaggcggcggcggatccgccgccgccgaagtgcagctggaggaatccggtggcggcctggtgcagcccggcggctccctgagactgtcttgcgccgcttccggcttcaccttttccgactacgccatgtcctgggtgcggcaggctcctggaaagggcctggaatgggtgtctgccatcagatggcactcttcctggacctactacgccgactccgtgaagggacggttcaccatctctcgggacaactccaagaatacactgtacctgcagatgaactccctgagggctgaggacaccgctgtgtactactgtgccaagatgaaatctaccagacactggatcgacgattggggccaaggcaccctggtgaccgtgtcctctggcggcggcggcagcggcggaggaggctctggcggcggaggctcttccgagctgacccaggatccagccgtgtccgtggccctgggccagacagtgcggatcacctgtcagggcgactccctgcggtcctattacgcctcttggtaccagcagaagcctggccaggcccctgtgctggtgatctacggcaagaacaaccggccttcaggcatccctgacagattttccggaagctcctctggcaacaccgctagccttaccatcacaggcgcccaggctgaagatgaagccgattactactgccagtcgtacgacagcagcagctaccactacgtgttcggcggcggcaccaagctgactgtcctc

SEQ ID NO: 153 - protein, 579 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSDLTVDKSRWQQGNVFSCSVMHEALHNHYTQESLSLSPGKGGGGSAAAEVQLEESGGGLVQPGGSLRLSCAASGFTFSDYAMSWVRQAPGKGLEWVSAIRWHSSWTYYADSVKGRFTISRDNSKNTLYLQMNSLRAEDTAVYYCAKMKSTRHWIDDWGQGTLVTVSSGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCQSYDSSSYHYVFGGGTKLTVL

SEQ ID NO: 154 - DNA, 1737 bp, synthetic construct
  source 1..1737: mol_type = other DNA
gcctctaccaaaggcccctccgtgttccctctggccccctctagcaaatctacctctggaggcacagccgctctgggctgtctggtcaaggactacttccccgaacctgtgaccgtgtcctggaactctggagctctgacctctggagtgcacacctttcctgccgtgctgcagtcttctggactgtattccttgagctccgtcgtgaccgtgccttcctcctccctgggaacacagacctacatctgcaacgtgaaccacaagccatctaataccaaagtggacaagaaggtcgagcctaagtcctgcgacaagacccatacctgccctccttgtcctgctcctgagctgctgggcggcccttcggtgtttctgttcccccccaagcccaaggacaccctgatgatctcccgcacacctgaagtgacctgcgtggtggtggatgtgtctcacgaggatcctgaagtgaagttcaactggtatgtggatggcgtggaagtgcacaacgccaaaaccaagcctagagaggaacagtacgcctctacctacagagtagtcagcgtgctgacagttctgcaccaggattggctgaacggcaaagagtacaagtgcaaggtgtccaacaaggctctgcccgctcctatcgagaagaccatcagtaaggccaagggccagcctcgggagcctcaggtgtacacactgcctccatctcgggacgaactgaccaagaaccaggtgtctctgacctgcctggtgaaaggattctacccttccgacatcgccgtggaatgggagtccaacggccagccagagaacaactacaagaccacccctcctgtgctggactccgacggctccttcttcctgtactctgacctgaccgtggacaagtctagatggcagcaaggcaacgtgttttcttgctccgtgatgcacgaggccctgcataatcactacacccaagagtctctgtctctgtcccctggaaagggcggaggtggctctgccgccgctgaggtgcagctgaaggaatccgggggcggcctggtgcagcctggtggcagcctgagactgagttgcgctgcctccggctttaccttctccgattatgccatgtcctgggtgcggcaagctcctggcaaaggactggagtgggtatccgccatcagatggcactcctcctggacctattacgccgattctgtgaagggcagattcaccatctccagagacaactccaagaataccctctacctgcagatgaacagcctgcgggccgaggacacagccgtgtactactgcgctaagatgaagagcaccagacactggatcgacgactggggccagggcaccctggtcaccgtgtctagcggaggcggtggatctggtggtggcggatcaggtggaggcgggagctccgagctgacccaggaccctgctgtgtccgtggccctgggccagactgtgcggatcacctgtcagggcgactcccttcggtcctactacgcatcctggtaccagcagaagcccggacaggcgcctgttctggtgatctacggcaagaacaacagaccttccggcatccctgacagattctctggttcgagcagtggcaacaccgcttctctgacaatcacaggagctcaggccgaggatgaggccgactactactgccagagctacgacagctccagctaccactacgtattcggcggcggcaccaagctgacggtgctc

SEQ ID NO: 155 - protein, 579 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSDLTVDKSRWQQGNVFSCSVMHEALHNHYTQESLSLSPGKGGGGSAAAEVQLKESGGGLVQPGGSLRLSCAASGFTFSDYAMSWVRQAPGKGLEWVSAIRWHSSWTYYADSVKGRFTISRDNSKNTLYLQMNSLRAEDTAVYYCAKMKSTRHWIDDWGQGTLVTVSSGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCQSYDSSSYHYVFGGGTKLTVL

SEQ ID NO: 156 - DNA, 1737 bp, synthetic construct
  source 1..1737: mol_type = other DNA
gcttctactaagggcccctccgtgttccctctggccccatcttccaagagcacctctggaggcaccgctgccctgggctgcctggtgaaagactacttccccgagcctgtaaccgtttcttggaacagcggagctctgacctccggtgtgcacacttttcccgccgtgctgcagtcctctggcctgtactcgctgagctcagtcgtgaccgtgccttcctcctctctgggcacccagacctacatctgcaacgtgaaccacaagccttccaacaccaaagtggacaagaaggtggaacctaagtcctgcgacaagacccacacatgtcctccctgtcctgcccccgaactgctgggaggcccatccgtgtttctgttccctcccaagcctaaggataccctgatgatctctcggaccccagaagttacctgtgtggtcgtggatgtatctcacgaagatcctgaggtgaagttcaattggtatgtggacggcgtggaagtgcacaacgccaagaccaagcctagagaggaacagtacgcctccacctaccgggtggtgtccgtgctgaccgtgctgcatcaggactggctgaacggcaaagagtacaagtgcaaggtgtctaacaaggccctgcctgcacctatcgagaagacaatctccaaggccaagggccagccccgggagcctcaggtgtacaccctgcctccatctcgggacgagctgaccaagaaccaggtgtctctgacatgcctggtgaaaggcttctacccgtccgacatcgccgtggagtgggagtccaacggccaacccgagaacaactacaaaacaacacctcctgtgctggactctgatggcagcttcttcctgtatagcgacctgacagtggataagtccagatggcagcagggcaatgtgttctcctgctctgtgatgcacgaggccctgcataaccactacacccaagagtctctgtccctgtcccctggcaagggcggaggtggctctggaggctccgaagtgcagctgctggaatccggaggcggcctagtccagcctggcggctctttgagactgtcgtgcgcggcttcgggcttcaccttctctgattacgccatgtcctgggtcagacaggctcctggcaagggactggagtgggtgtccgccatcagatggcactccagctggacctattacgccgattcggtgaagggcagatttaccatctcccgcgacaacagtaaaaataccctgtacctgcagatgaactctctgcgggctgaggacacagccgtgtactactgcgccaagatgaagtctaccagacactggatcgacgactggggccagggcaccctggtgaccgtcagctccggaggcggtggatctggtggtggcggatcaggtggaggcgggagctccgaattaacccaggatccagctgtgagcgtggccctgggccagaccgtgagaatcacctgtcagggcgactccctgcggtcctactacgcctcctggtaccagcaaaaacccggccaagcccctgtgctggtgatctacggcaagaacaaccggcctagcggcatccctgacagattctccggctcgtcttctggcaacaccgcctccctgacaatcaccggcgcccaggctgaggacgaggctgactactactgccagtcttacgactcctcctcataccactacgtgttcggcggcggcaccaagctgaccgtgctg

SEQ ID NO: 157 - protein, 579 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSDLTVDKSRWQQGNVFSCSVMHEALHNHYTQESLSLSPGKGGGGSGGSEVQLLESGGGLVQPGGSLRLSCAASGFTFSDYAMSWVRQAPGKGLEWVSAIRWHSSWTYYADSVKGRFTISRDNSKNTLYLQMNSLRAEDTAVYYCAKMKSTRHWIDDWGQGTLVTVSSGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCQSYDSSSYHYVFGGGTKLTVL

SEQ ID NO: 158 - DNA, 987 bp, synthetic construct
  source 1..987: mol_type = other DNA
gccagcaccaagggcccttctgtctttcctctggcccctagctctaagtccacatctggcggcacagctgctctgggctgcctggtgaaggactacttccccgagcctgtgacagtctcttggaacagcggcgccctgacctccggcgtgcacacctttcctgccgtgcttcaatcctccggcctgtatagcctctcttctgtcgtgaccgtgccatcttcctctctgggcacccagacctacatctgcaacgtgaaccacaagccatccaacaccaaagtggacaaaaaggtggaacccaagtcctgcgacaagacccatacctgtcctccatgccctgctcccgagctgctgggaggcccttccgtgttcctgttccctcctaagcctaaagataccctgatgatctcccggacccctgaggtgacctgcgtggtggtcgatgtgtcccacgaggaccccgaagtgaagttcaactggtacgtggatggcgtggaagtgcacaacgccaagaccaagcctcgggaagagcagtacgcttctacctatagagtggtgtccgtgctgaccgtgctgcaccaggactggctgaacggaaaagagtacaagtgcaaggtctccaacaaggctctgcctgctcctatcgagaagacaatctccaaggccaagggccagcctagagagccccaagtgtacacactgcctccttcccggaagaagctgaccaagaaccaggtcagcctgacctgtctggtgaaaggcttctaccctagcgacatcgccgtggagtgggagtctaatggacagcctgagaacaactataagaccaccccccccgtgctgaagtctgacggctctttcttcctgtactccaagctgacagtggacaaatctcggtggcagcagggcaacgtgttctcctgctccgtgatgcacgaggccctgcataatcactacacccagaagtctctgtctctgtcccctggc

SEQ ID NO: 159 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRKKLTKNQVSLTCLVKGFYPSDIAVEWESNGQPENNYKTTPPVLKSDGSFFLYSKLTVDKSRWQQGNVFSCSVMHEALHNHYTQKSLSLSPG

SEQ ID NO: 160 - DNA, 1737 bp, synthetic construct
  source 1..1737: mol_type = other DNA
gcctctaccaagggaccttctgtgtttcctctggctccttcttccaagtccacctctgggggtaccgctgctctaggctgcctggtgaaggactacttccctgaacctgtgacggtgtcttggaactctggagctctgacctccggcgtacatacatttcctgctgttctgcagtcctctggcctgtactcgctgtcttctgtggtcacagtcccctctagctctctcggtacacagacctacatctgcaacgtgaaccacaagccatctaataccaaggttgataagaaggtggaaccaaaatcttgcgacaagacccacacctgccctccttgtcctgcccccgaactgctgggaggccctagcgtgttcctgttccctcctaagcccaaggacaccctgatgatctctagaacacccgaagtgacctgcgtggtggtggacgtgtcccatgaggatcctgaagtcaagttcaactggtacgtggacggcgtggaagtgcacaacgccaagacaaagcctcgcgaagagcagtacgcttccacctaccgggtggtgtccgtgctgacagtcctgcaccaggattggttgaacggcaaagagtacaagtgcaaggtgtccaacaaggccctgcccgctcctatcgagaagaccatttccaaggctaaaggacagcctagggagcctcaggtttacaccctgcctccaagccgggacgagctgaccaagaaccaggtgtccctgacgtgtctggtcgaaggcttctaccccagcgacatcgccgtcgaatgggagagcaatggccagcccgagaacaactacaagaccacccctcctgtgctggactccgatggctccttcttcctgtactccgacctgaccgtggacaagtcccggtggcagcagggcaatgtgttctcctgctccgtgatgcacgaggctctgcacaatcactacacacaagagtccctgtctctgagccctggaaaaggcggaggtggctctgctgctgcagaggtgcagctgctggaatccggaggtggcctcgtgcagcctggcggcagcctgcggctgtcctgtgccgcctctggctttaccttctctgactatgccatgtcctgggtgcggcaggcccctggcaagggcctggagtgggtgagtgccatcagatggcactccagctggacctactacgctgattccgtgaagggacggttcaccatcagccgggataactccaagaacaccctgtacctgcagatgaactccctgcgggctgaggatacagccgtgtactactgcgccaagatgaagtccaccagacactggatcgatgactggggccagggcaccctggtgaccgtgtcttctggaggcggtggatctggtggtggcggatcaggtggaggcgggagctccgagctgacacaagaccccgccgtgtcggtggccctgggccagaccgtgcggatcacctgccagggcgactccctgagatcctactacgcctcttggtaccagcagaagcccggccaagccccagtgctggtgatctacggcaagaacaacagaccttccggcatccctgacagattttctggcagctcttccggcaacaccgcctctctgaccatcaccggagctcaggccgaggacgaggccgactactactgtcagtcttacgactcctcgtcctaccactacgtgttcggcggcggcaccaagctgactgtgctg

SEQ ID NO: 161 - protein, 579 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVEGFYPSDIAVEWESNGQPENNYKTTPPVLDSDGSFFLYSDLTVDKSRWQQGNVFSCSVMHEALHNHYTQESLSLSPGKGGGGSAAAEVQLLESGGGLVQPGGSLRLSCAASGFTFSDYAMSWVRQAPGKGLEWVSAIRWHSSWTYYADSVKGRFTISRDNSKNTLYLQMNSLRAEDTAVYYCAKMKSTRHWIDDWGQGTLVTVSSGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCQSYDSSSYHYVFGGGTKLTVL

SEQ ID NO: 162 - DNA, 1737 bp, synthetic construct
  source 1..1737: mol_type = other DNA
gcttccaccaagggccctagcgtgtttcctctggcccctagctccaagagtacctccggcggcaccgcagctctgggctgcctcgtgaaggactacttccctgaacccgtgacagtgtcttggaactcgggcgctctgaccagtggagtgcacacctttccagccgtgctgcagtcttccggcctgtactccctgagctctgtggtgacagtgccttctagctctctgggcacacagacctacatctgcaacgtgaaccacaaaccctccaacacgaaagtggacaagaaggtggaacctaagtcctgcgacaagacccatacctgccctccttgccctgctcctgagctgctgggcgggccttccgtgtttctgttcccccctaaacccaaggacaccctaatgatctccagaacccctgaagttacatgcgtggtcgtggacgtgtcccacgaggatccagaggtgaagttcaactggtatgtggatggcgtggaagtgcacaacgccaagaccaagccaagagaagagcagtacgcgtccacctacagagtggtgtccgtgctcaccgtgctgcaccaggactggctgaacggcaaagagtacaagtgcaaggtgtccaacaaggccctgccagctcctatcgagaagaccatctctaaggctaagggccaacctagagagcctcaggtgtacaccctgcctccctctcgggacgagctgacaaagaaccaggtctccctgacctgcctggtcgagggcttctacccctccgacatcgccgtcgagtgggagtccaatggccagcccgagaacaactacaagacaacacctcctgtgctggactccgacggctccttcttcctgtactccgacctgaccgtggataaatctcggtggcagcagggcaacgtgttctcctgctccgtgatgcacgaggctctgcataatcactacacccaagagtctctgagcctgtctcctggaaagggcggaggcggatccgccgctgctgaagtgcagctgctggagtctggcggaggcctggtgcagcctgggggctcactgcggctgtcttgcgccgctagcggctttaccttcagcgattacgccatgtcctgggtgcggcaggccccaggcaagtgcctggaatgggtgtctgccatcagatggcactcctcctggacctactacgctgattctgtgaaaggccggttcaccatttcccgggataatagcaagaataccctgtacctgcagatgaactctctgcgggctgaggacaccgccgtgtactactgcgccaagatgaagtctaccagacactggatcgacgactggggccaaggcaccctggtcaccgtgtcctccggcggcggaggatccggtggcggcgggtctggcggaggcggctcttccgaactgacccaggatcctgccgtgtccgtggccctgggacagacagtacggatcacctgtcagggcgactccctgcgctcttactacgcctcttggtaccagcagaagcctggccaggcccctgtgctggtgatctatggcaagaacaacagaccttctggcatccctgacagattctccggaagctccagcggcaacaccgcttctctcaccatcaccggcgctcaggccgaagatgaggccgactactactgccagtcctacgactcttcctcctaccactacgtgttcggctgtggcaccaagctgaccgtgctg

SEQ ID NO: 163 - protein, 579 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVEGFYPSDIAVEWESNGQPENNYKTTPPVLDSDGSFFLYSDLTVDKSRWQQGNVFSCSVMHEALHNHYTQESLSLSPGKGGGGSAAAEVQLLESGGGLVQPGGSLRLSCAASGFTFSDYAMSWVRQAPGKCLEWVSAIRWHSSWTYYADSVKGRFTISRDNSKNTLYLQMNSLRAEDTAVYYCAKMKSTRHWIDDWGQGTLVTVSSGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCQSYDSSSYHYVFGCGTKLTVL

SEQ ID NO: 164 - DNA, 987 bp, synthetic construct
  source 1..987: mol_type = other DNA
gccagcaccaagggcccttctgtctttcctctggcccctagctctaagtccacatctggcggcacagctgctctgggctgcctggtgaaggactacttccccgagcctgtgacagtctcttggaacagcggcgccctgacctccggcgtgcacacctttcctgccgtgcttcaatcctccggcctgtatagcctctcttctgtcgtgaccgtgccatcttcctctctgggcacccagacctacatctgcaacgtgaaccacaagccatccaacaccaaagtggacaaaaaggtggaacccaagtcctgcgacaagacccatacctgtcctccatgccctgctcccgagctgctgggaggcccttccgtgttcctgttccctcctaagcctaaagataccctgatgatctcccggacccctgaggtgacctgcgtggtggtcgatgtgtcccacgaggaccccgaagtgaagttcaactggtacgtggatggcgtggaagtgcacaacgccaagaccaagcctcgggaagagcagtacggctctacctatagagtggtgtccgtgctgaccgtgctgcaccaggactggctgaacggaaaagagtacaagtgcaaggtctccaacaaggctctgcctgctcctatcgagaagacaatctccaaggccaagggccagcctagagagccccaagtgtacacactgcctccttcccggaagaagctgaccaagaaccaggtcagcctgacctgtctggtgaaaggcttctaccctagcgacatcgccgtggagtgggagtctaatggacagcctgagaacaactataagaccaccccccccgtgctgaagtctgacggctctttcttcctgtactccaagctgacagtggacaaatctcggtggcagcagggcaacgtgttctcctgctccgtgatgcacgaggccctgcataatcactacacccagaagtctctgtctctgtcccctggc

SEQ ID NO: 165 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRKKLTKNQVSLTCLVKGFYPSDIAVEWESNGQPENNYKTTPPVLKSDGSFFLYSKLTVDKSRWQQGNVFSCSVMHEALHNHYTQKSLSLSPG

SEQ ID NO: 166 - DNA, 1737 bp, synthetic construct
  source 1..1737: mol_type = other DNA
gcctctaccaagggaccttctgtgtttcctctggctccttcttccaagtccacctctgggggtaccgctgctctaggctgcctggtgaaggactacttccctgaacctgtgacggtgtcttggaactctggagctctgacctccggcgtacatacatttcctgctgttctgcagtcctctggcctgtactcgctgtcttctgtggtcacagtcccctctagctctctcggtacacagacctacatctgcaacgtgaaccacaagccatctaataccaaggttgataagaaggtggaaccaaaatcttgcgacaagacccacacctgccctccttgtcctgcccccgaactgctgggaggccctagcgtgttcctgttccctcctaagcccaaggacaccctgatgatctctagaacacccgaagtgacctgcgtggtggtggacgtgtcccatgaggatcctgaagtcaagttcaactggtacgtggacggcgtggaagtgcacaacgccaagacaaagcctcgcgaagagcagtacggctccacctaccgggtggtgtccgtgctgacagtcctgcaccaggattggttgaacggcaaagagtacaagtgcaaggtgtccaacaaggccctgcccgctcctatcgagaagaccatttccaaggctaaaggacagcctagggagcctcaggtttacaccctgcctccaagccgggacgagctgaccaagaaccaggtgtccctgacgtgtctggtcgaaggcttctaccccagcgacatcgccgtcgaatgggagagcaatggccagcccgagaacaactacaagaccacccctcctgtgctggactccgatggctccttcttcctgtactccgacctgaccgtggacaagtcccggtggcagcagggcaatgtgttctcctgctccgtgatgcacgaggctctgcacaatcactacacacaagagtccctgtctctgagccctggaaaaggcggaggtggctctgctgctgcagaggtgcagctgctggaatccggaggtggcctcgtgcagcctggcggcagcctgcggctgtcctgtgccgcctctggctttaccttctctgactatgccatgtcctgggtgcggcaggcccctggcaagggcctggagtgggtgagtgccatcagatggcactccagctggacctactacgctgattccgtgaagggacggttcaccatcagccgggataactccaagaacaccctgtacctgcagatgaactccctgcgggctgaggatacagccgtgtactactgcgccaagatgaagtccaccagacactggatcgatgactggggccagggcaccctggtgaccgtgtcttctggaggcggtggatctggtggtggcggatcaggtggaggcgggagctccgagctgacacaagaccccgccgtgtcggtggccctgggccagaccgtgcggatcacctgccagggcgactccctgagatcctactacgcctcttggtaccagcagaagcccggccaagccccagtgctggtgatctacggcaagaacaacagaccttccggcatccctgacagattttctggcagctcttccggcaacaccgcctctctgaccatcaccggagctcaggccgaggacgaggccgactactactgtcagtcttacgactcctcgtcctaccactacgtgttcggcggcggcaccaagctgactgtgctg

SEQ ID NO: 167 - protein, 579 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLTCLVEGFYPSDIAVEWESNGQPENNYKTTPPVLDSDGSFFLYSDLTVDKSRWQQGNVFSCSVMHEALHNHYTQESLSLSPGKGGGGSAAAEVQLLESGGGLVQPGGSLRLSCAASGFTFSDYAMSWVRQAPGKGLEWVSAIRWHSSWTYYADSVKGRFTISRDNSKNTLYLQMNSLRAEDTAVYYCAKMKSTRHWIDDWGQGTLVTVSSGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCQSYDSSSYHYVFGGGTKLTVL

SEQ ID NO: 168 - DNA, 987 bp, synthetic construct
  source 1..987: mol_type = other DNA
gctagcaccaagggaccttccgtgtttcctctggccccttcttctaaatctacctccggcggcaccgctgctctgggctgcctggtgaaggactacttccctgagcctgtgaccgtgtcctggaactccggcgccctgacctctggggtgcacaccttcccagctgtgctgcagtcctccggcctgtattccctgtcttctgtggtgaccgttcccagctcttccttgggcacacagacctacatctgcaacgtgaatcacaagccctccaatacaaaagtggataagaaggtggaacccaagtcctgcgacaagacacatacctgtcctccttgccctgctcctgagctgctgggcggcccttctgtgtttctgttcccccctaaacccaaggataccctgatgatctctagaaccccggaagtgacctgcgtggtcgtggacgtgtctcacgaggaccctgaagtcaagttcaactggtacgtggacggcgtggaggtgcataacgccaagaccaagcctcgcgaggaacagtacgcctccacctacagagtggtgagtgtgctgaccgtgctgcaccaggactggctgaacggcaaagagtacaagtgcaaggtgtccaataaggctctgcccgccccaatcgagaagacaatctccaaggccaaaggccagcctagagagcctcaagtgtacaccctccctccatctcgggacgagctgaccaagaaccaggtgagtctaacctgtctcgtcgagggcttctacccttccgacatcgccgtggagtgggagtccaacggccaacccgagaacaactacaagaccacccctcctgtgctggactccgatggctccttcttcctgtactccgacctgaccgtggacaagtccaggtggcagcagggcaacgtgttctcctgctccgtgatgcacgaggctctgcacaaccactacacccaggaatctctgtccctgtctcctgga

SEQ ID NO: 169 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVEGFYPSDIAVEWESNGQPENNYKTTPPVLDSDGSFFLYSDLTVDKSRWQQGNVFSCSVMHEALHNHYTQESLSLSPG

SEQ ID NO: 170 - DNA, 1737 bp, synthetic construct
  source 1..1737: mol_type = other DNA
gctagtacaaaaggcccctccgtgttccctctggctcccagttccaagtccacctctggcggcaccgctgctctgggctgtctggtgaaggactacttcccagagcccgtgacagtgtcctggaactccggcgccctgacctccggcgtgcataccttccccgccgtgctgcagtccagcggcctgtactccctgtcttccgtggtgacagtgcctagcagctctctgggcacccagacctacatctgcaacgtgaaccacaagccttctaacaccaaggtggacaagaaagtggagcctaagtcctgcgacaagacccacacctgccctccttgtcccgcgcctgaactgctgggcgggccttctgtgtttctgttcccacctaagcccaaggataccctgatgatcagcagaacccctgaggtgacctgcgtggttgtggacgtgtcccatgaagatcctgaagtgaagttcaactggtatgtggatggcgtggaagtgcacaacgccaaaaccaagcctcgggaagagcagtacgcctccacctacagagtggtgagcgtgctgaccgtgctgcaccaggattggctgaacggcaaagagtacaagtgcaaggtgtccaacaaggccctgcctgcccccatcgagaagaccatcagcaaggctaagggccagcctagagagcctcaggtgtacaccctgcctccatctcggaagaagctgaccaagaaccaggtatccctcacatgcctggtgaagggcttctacccttccgacatcgccgtggagtgggagtccaacggccagcctgagaacaactacaagaccacccctcctgtgctgaagtccgacggcagcttcttcctgtacagcaagctgaccgtggataagtctagatggcagcagggcaatgtgttctcctgctccgtgatgcacgaggctctgcacaatcactacacacaaaagtctctgagcctgtctcctggaaaaggcggaggtggctctgccgccgctgaagtgcagctgctggaatctggcggcggactggtgcagcccggcggatctctgagactgtcctgtgccgccagcggcttcaccttctccgattacgccatgagctgggtgcgccaagccccaggcaagggcctggaatgggtctctgccatccggtggcactctagctggacctactatgccgattctgtaaaaggccggtttaccatttccagagataactctaagaacaccctgtacctgcagatgaactccctgcgggccgaggacaccgctgtgtactactgcgccaagatgaaatctaccagacactggatcgacgactggggccagggcactctcgtgacagtgtcttctggaggcggtggatctggtggtggcggatcaggtggaggcgggagctccgagctgacccaggaccctgcagtgtctgtggctctgggccagactgtgcggatcacctgccagggcgactccctgcgatcttattatgcttcctggtaccagcagaagcccggacaagctcccgtgctggtgatctacggcaaaaacaacagaccttccggcatccctgacaggttttctggatcttcctccggcaacaccgcctcactcacaatcaccggagctcaggccgaggacgaggccgactactactgtcagtcttacgactctagctcttaccactacgtgttcggcggaggaaccaagctgaccgttctg

SEQ ID NO: 171 - protein, 579 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRKKLTKNQVSLTCLVKGFYPSDIAVEWESNGQPENNYKTTPPVLKSDGSFFLYSKLTVDKSRWQQGNVFSCSVMHEALHNHYTQKSLSLSPGKGGGGSAAAEVQLLESGGGLVQPGGSLRLSCAASGFTFSDYAMSWVRQAPGKGLEWVSAIRWHSSWTYYADSVKGRFTISRDNSKNTLYLQMNSLRAEDTAVYYCAKMKSTRHWIDDWGQGTLVTVSSGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCQSYDSSSYHYVFGGGTKLTVL

SEQ ID NO: 172 - DNA, 987 bp, synthetic construct
  source 1..987: mol_type = other DNA
gcctctacaaagggcccttccgtgtttcctctggccccctcctccaagtccacctctggaggcacagctgctctgggctgcctggtcaaggactacttccctgagcctgtgaccgtgtcttggaactccggcgctctgacctctggcgtgcataccttccctgctgtgctgcagtcctctggcctgtactccctctcttctgtcgtgaccgtgccttcttccagcctgggaacccagacctacatctgcaacgtgaaccacaagccaagcaacaccaaggtggacaagaaagtggaacctaagtcctgcgataaaacccacacctgccctccatgtcctgcccctgaactgctgggcggaccatccgtgtttctgttcccccctaagcccaaggacaccctgatgatctccaggacccccgaggtgacttgcgtggtcgtagacgtgagccacgaggaccccgaagtgaagttcaactggtacgtggatggcgtggaagtgcacaacgccaagaccaagcctagagaggaacagtacggcagcacctacagagtggtgtccgtgctgacagtgctgcaccaggactggctgaacggcaaagagtataagtgcaaggtctccaacaaggctttgcctgctccaatcgagaagaccatctccaaggccaaaggacagccccgcgaacctcaggtgtacaccctccctccttctcgggacgagctgaccaagaaccaggtcagcctgacatgtctggtggagggcttctacccttccgatatcgccgtggagtgggagtccaatggccagcctgagaacaactacaagaccacccctcccgtgctggactctgacggctccttcttcctgtattctgacctgaccgtggataagtctagatggcagcagggcaacgtgttcagctgctccgtgatgcacgaggccctgcataatcactacacacaagagtctctgagcctgtcccctgga

SEQ ID NO: 173 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLTCLVEGFYPSDIAVEWESNGQPENNYKTTPPVLDSDGSFFLYSDLTVDKSRWQQGNVFSCSVMHEALHNHYTQESLSLSPG

SEQ ID NO: 174 - DNA, 1737 bp, synthetic construct
  source 1..1737: mol_type = other DNA
gccagcaccaagggcccctccgtgttccctctggccccttcctccaaatctacctccggcggaacggctgccctgggctgcctggttaaggactacttccccgagcccgtgacagtgtcctggaactccggagctctgaccagcggcgtgcatacctttcctgctgtgctgcagtcttccggcctgtactctctgtcctctgtggtgaccgtgccttccagcagcctgggcacccagacctacatctgcaacgtgaaccacaagccttccaataccaaggtggacaagaaagtggaacctaagtcctgcgacaagacacacacctgccctccttgccctgctcccgaactgttgggcggcccttccgtgtttctgttcccgcccaagcctaaggacaccctgatgatctctagaacccctgaggtgacctgcgtggtggtggacgtgtctcacgaggaccctgaagtgaagttcaactggtatgtggatggcgtcgaggtgcacaatgccaagaccaagcctagagaggaacagtacggctctacctacagagtggtgtctgtgctgaccgtcctgcaccaggactggctgaacggcaaagagtacaagtgcaaggtgtccaacaaggccctgcccgcccctatcgagaagaccatcagcaaggctaagggacagcctagagagcctcaggtgtacaccctgcctccttctcggaagaagctgaccaagaaccaagtgtccctgacttgtctggtgaaaggcttctatccatccgacatcgccgtggaatgggagtccaacggccagcctgagaacaactacaaaaccaccccacctgtgctgaagtccgacggctccttcttcctgtactccaagctgaccgtggacaagtctagatggcagcagggcaatgtgttctcctgctccgtgatgcacgaggctctgcacaaccattacacccagaagtctctatcgctctcgccaggcaaaggcggcggcggctccgccgctgccgaagttcagctgctggaatctggcggtggcctggtccaacctggcggctctctgcggctgtcctgtgctgcctctggcttcaccttctctgattacgccatgtcctgggtgcggcaggctcctggaaagggcctggagtgggtctctgccatccggtggcactcctcctggacctattacgccgattctgtgaagggaagattcaccatctccagggacaattccaagaacacactgtacctgcagatgaactccctgagagctgaggacaccgctgtgtactactgcgccaagatgaagtccaccagacactggatcgacgattggggccagggaacattagtgaccgtttcttccggcggcggcggatctggcggcggtggctcgggcggaggcggaagctctgagctgacccaggatccagccgtgtccgtggccctgggccagaccgtgcggatcacgtgtcagggcgactctctgcggtcttattacgccagctggtaccaacagaaacccggccaggctcctgtgctggtgatctacggcaagaacaaccggccttccggcatccctgacagattttctggctccagctctggcaacaccgcctcactgacaatcaccggcgctcaggccgaagatgaggccgactactactgccagtcctacgactccagttcttaccactacgtgttcggcggcggcaccaagctgacagtcctg

SEQ ID NO: 175 - protein, 579 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRKKLTKNQVSLTCLVKGFYPSDIAVEWESNGQPENNYKTTPPVLKSDGSFFLYSKLTVDKSRWQQGNVFSCSVMHEALHNHYTQKSLSLSPGKGGGGSAAAEVQLLESGGGLVQPGGSLRLSCAASGFTFSDYAMSWVRQAPGKGLEWVSAIRWHSSWTYYADSVKGRFTISRDNSKNTLYLQMNSLRAEDTAVYYCAKMKSTRHWIDDWGQGTLVTVSSGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCQSYDSSSYHYVFGGGTKLTVL

SEQ ID NO: 176 - DNA, 1737 bp, synthetic construct
  source 1..1737: mol_type = other DNA
gcttctacaaagggcccctctgtgtttcctctggccccatccagcaagtccacctctggaggcacagctgctctgggctgcctggtcaaggattacttcccagaacccgtgaccgtgtcttggaactctggcgccctgacctccggcgtgcatacctttcctgccgtgctgcaatccagcggcctgtactccttgtcctccgtggtcaccgtgccttctagttctctgggcacacagacctacatctgcaacgtgaaccacaagccctccaataccaaggtggacaagaaagtggaacctaaatcctgcgacaaaacgcacacctgtcctccttgccctgctcctgagctgctggggggcccttctgtgttcctgttccctcctaagcccaaggacaccctgatgatctctcggacccctgaagtcacctgcgtggtggtggacgtgtcccacgaggaccccgaggtcaagttcaactggtatgtggacggcgtggaagtgcacaacgccaagaccaagcctcgggaagagcagtacgcttctacctacagagtggtgtccgtgctgacagtcctgcaccaggactggctgaacggcaaagagtacaagtgcaaggtgtccaacaaggccctgcctgctcctattgagaagaccatctccaaggctaagggccaacccagagagcctcaggtgtataccctgcctccttccagaaagaagctgaccaagaaccaggtgtccctgacctgtctggtgaagggcttctacccttccgacatcgccgtggaatgggagtcgaacggccagcctgagaacaactacaagaccaccccccctgtgctgaagtctgatggctccttcttcctgtactccaaactgacagtggataagtcccgctggcagcagggcaacgtgttttcttgctccgtgatgcacgaggctctgcacaaccattacacacaaaaatctctgagtctgtcccctggcaaaggcggcggtggctcggccgctgctgaggtgcagctgctggaatccggcggaggactggtgcagcctgggggatccctgagactgtcctgcgctgcctctggcttcaccttctccgactacgccatgtcctgggtcagacaggcaccaggcaagggactggagtgggtgagcgccatcagatggcactctggctggacctattatgctgattctgtgaaaggccggttcaccatctccagagacaactccaagaataccctctacctgcagatgaactctctgagagccgaggataccgctgtgtactactgcgccaagatgaagtccaccagacactggatcgacgactggggccagggcacactggttaccgtgtcctctggcggtggcggctctggcggcggcggctcaggcggcggaggaagctccgagctgacccaggatcctgctgtgtccgtggccctgggacagaccgtgcggatcacctgtcagggcgactccctgcggtcctattacgcctcttggtaccagcagaagcctggccaggcccccgtgctggtgatctacggcaagaataaccggcctagtggcatccctgatagattcagcggctcttcgtctggtaacacagcttccctgacaatcaccggcgcccaggccgaggacgaggccgactactactgccagtcgtacgactcctcctcctaccactacgtgttcggcggcggcaccaagctgaccgtgctc

SEQ ID NO: 177 - protein, 579 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRKKLTKNQVSLTCLVKGFYPSDIAVEWESNGQPENNYKTTPPVLKSDGSFFLYSKLTVDKSRWQQGNVFSCSVMHEALHNHYTQKSLSLSPGKGGGGSAAAEVQLLESGGGLVQPGGSLRLSCAASGFTFSDYAMSWVRQAPGKGLEWVSAIRWHSGWTYYADSVKGRFTISRDNSKNTLYLQMNSLRAEDTAVYYCAKMKSTRHWIDDWGQGTLVTVSSGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCQSYDSSSYHYVFGGGTKLTVL

SEQ ID NO: 178 - DNA, 1737 bp, synthetic construct
  source 1..1737: mol_type = other DNA
gcttctaccaagggcccttccgtgttccccctggctccttcttccaagagcacctctggcggcaccgctgccctgggctgcctggtgaaggactacttccctgagcccgtgaccgtgtcctggaactccggtgctctgacatctggcgtacatacctttcctgcagtgctgcaatcttccggcctgtactccctgtcctctgtggtcaccgtgccttcgtcctccctgggcacccagacctacatctgcaacgtgaaccacaagccttccaataccaaggtggacaagaaagtggaacctaagtcctgcgacaagacccacacctgtcctccttgccccgctccagaactgctgggcggacctagtgtgtttctgttcccacctaaacccaaggacaccctgatgatctctcgcacccctgaggtgacctgcgtggtggtggacgtgtcccatgaggaccccgaagtgaagttcaactggtacgtggatggcgtggaagtgcacaacgccaagacaaagcctcgggaagagcagtacggcagcacctacagagtggtgtccgtgctgaccgtgctgcaccaggattggctgaacggcaaagagtacaagtgcaaggtgtccaacaaggccctgcctgctcctatcgagaagaccatctccaaagccaagggccagccaagagagccccaggtgtacaccctgcctccctctcggaagaagctgaccaagaaccaggtgagtctgacctgcctggtcaagggcttctacccctccgacatcgccgtcgagtgggagtctaatggccagcctgagaacaactacaagaccacccctcctgtgctgaagtccgacggctccttcttcctgtactctaagctgactgtggataagtctcggtggcagcagggcaacgtgttctcctgctccgtgatgcacgaggctctgcacaaccactatacacaaaaatctctgtctctgtctcccggaaaaggcggcggtggctctgccgccgccgaagtccagctcctggaatccggcggcggcctggtgcagcctggcggatctctgagactgtcttgtgccgccagcggctttaccttctctgattacgccatgtcctgggtgagacaggctcctggcaaaggcctggagtgggtttctgccatcagatggcactccggctggacctactacgccgattcggtcaagggccggttcaccatttccagagataattccaagaatacactgtacctgcagatgaactctctgagagctgaagataccgctgtgtactactgtgccaagatgaaatccacacggcactggatcgacgactggggccagggcaccctggtgaccgtgtctagcggaggcggcggctctggcggcggcggaagcggaggcggaggatcctccgagctgacccaggaccctgctgtgtctgtggccctcggccagacagtgcggatcacctgtcagggcgactccctgagatcttattacgcctcttggtaccagcagaagcctggccaggctccagttctggtgatctacggcaagaacaacagaccttccggcatccctgacagattctccggcagttcttccggcaacaccgcctctctgacaatcaccggcgcccaggccgaggacgaggccgattactactgccagtcctacgactcatcctcctaccactacgtgttcggcggcggcaccaagctgaccgtgctg

SEQ ID NO: 179 - protein, 579 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRKKLTKNQVSLTCLVKGFYPSDIAVEWESNGQPENNYKTTPPVLKSDGSFFLYSKLTVDKSRWQQGNVFSCSVMHEALHNHYTQKSLSLSPGKGGGGSAAAEVQLLESGGGLVQPGGSLRLSCAASGFTFSDYAMSWVRQAPGKGLEWVSAIRWHSGWTYYADSVKGRFTISRDNSKNTLYLQMNSLRAEDTAVYYCAKMKSTRHWIDDWGQGTLVTVSSGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCQSYDSSSYHYVFGGGTKLTVL

SEQ ID NO: 180 - DNA, 990 bp, synthetic construct; identical to SEQ ID NO: 94
  source 1..990: mol_type = other DNA

SEQ ID NO: 181 - protein, 330 aa, synthetic construct; identical to SEQ ID NO: 95

SEQ ID NO: 182 - DNA, 1737 bp, synthetic construct
  source 1..1737: mol_type = other DNA
gccagcaccaagggcccttctgtgttccctctggccccatcctccaagagcacctccggaggcaccgctgccctgggctgcctggtgaaggactacttccccgagccagtgaccgtgtcctggaactctggagctctgacatctggcgtgcacacgttccccgccgtcctccagtcctctggcctgtactccctgtccagcgtggtgaccgtgccttctagctccctgggaacccagacttacatttgcaacgtgaaccacaagccttccaacaccaaggtggataagaaagtggaacctaagtcttgcgacaagacacacacctgtcctccatgccctgctcctgagctgctgggcggaccttcggtttttctgttccctccaaagcccaaggacaccctgatgatctctaggacccccgaggtaacctgcgtggtggtggacgtgagccacgaggatcctgaggtgaaattcaactggtacgtcgatggcgtggaagtgcataatgccaaaaccaagcctagagaggaacagtacgcctctacctacagagtggtttccgtgctgaccgtgctgcaccaggattggctgaacggcaaagagtacaagtgcaaggtgtctaacaaggctctgcctgctcctatcgagaagaccatcagcaaggccaagggccagcccagagagcctcaagtgtacacactgcccccttctcggaaggaactgaccaagaaccaggtgtccttaacctgtctcgtgaagggcttctacccttccgacatcgccgtcgagtgggagtccaacggccagcccgagaacaactacaagaccaccccccctgtgctcaagtccgatggctccttcttcctgtacagcaaactgaccgtggacaagagccggtggcagcaaggcaatgtgttttcctgctccgtgatgcatgaggccctgcacaatcactacacccaaaagtccctgtccctgtctcctggaaagggcggaggtggctctgccgctgccgaagtgcagctgctggaatccgggggcggcctggtccagcccggaggttccctgagactgtcttgtgctgcttctggcttcaccttttccgactacgccatgtcttgggtccggcaggctcctggcaaaggcctggagtgggtgtccgccatccggtggcactctggctggacttactacgccgactccgtgaagggccggttcaccatctccagagataactctaagaacaccctgtacctgcagatgaactctctgagagccgaagataccgccgtgtattactgcgccaagatgaaatccacccggcactggatcgacgactgggggcagggcaccctggtgaccgtgtcctcgggaggcggtggatctggtggtggcggatcaggtggaggcgggagctctgaactgacccaggaccctgctgtgtccgtggccctgggccagaccgtgcggatcacatgccagggcgattccttgcgctcctattacgcctcttggtaccagcagaagcctggccaagcccctgtgctggtgatctacggcaagaacaacagaccttccggcatccctgacagattctccgggtcttcttccggcaacaccgctagcctgaccatcaccggcgctcaggccgaggacgaggctgactattactgtcagtcttacgactcttcttcctaccactacgtgttcggcggcggcacaaagctgacagtgctg

SEQ ID NO: 183 - protein, 579 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRKELTKNQVSLTCLVKGFYPSDIAVEWESNGQPENNYKTTPPVLKSDGSFFLYSKLTVDKSRWQQGNVFSCSVMHEALHNHYTQKSLSLSPGKGGGGSAAAEVQLLESGGGLVQPGGSLRLSCAASGFTFSDYAMSWVRQAPGKGLEWVSAIRWHSGWTYYADSVKGRFTISRDNSKNTLYLQMNSLRAEDTAVYYCAKMKSTRHWIDDWGQGTLVTVSSGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCQSYDSSSYHYVFGGGTKLTVL

SEQ ID NO: 184 - DNA, 990 bp, synthetic construct
  source 1..990: mol_type = other DNA
gcctccaccaaaggcccttctgtctttcctctggccccttcttccaagtccacctctggcggcaccgccgctctgggctgcctggtcaaggactacttccctgaacctgtgaccgtgtcctggaactccggcgctctgacctccggcgtgcacaccttcccagctgtgctgcagtccagcggactctatagcctgtcctccgtggtgaccgtgccttctagctctctgggcacccagacctacatctgcaacgtgaaccacaagccctccaacaccaaggtggataagaaagtggaacccaagtcctgcgacaaaacccatacgtgccctccatgtcctgcccccgaactgctgggaggccccagcgtgtttctgttcccacctaagcccaaggacaccctgatgatctcccggacccctgaggtgacctgcgtggtcgtggacgtgtcgcacgaggaccccgaagtgaagttcaactggtacgtggatggcgtcgaggtgcacaacgccaagacaaagccacgcgaggaacagtacgcttctacctacagagtggtgtctgtgctgacagtgctgcaccaggactggctgaacggcaaagagtacaagtgcaaggtgagcaacaaggctctgcctgctcctatcgagaagaccatttctaaggccaaaggccaacctagagagcctcaggtgtacacactgcctccttctcgggatgagctgaccaagaaccaggtttccctcacatgtctggtggaaggcttctacccttccgacatcgccgtggagtgggagtctaatggccagcctgagaacaactacaagaccaccccccctgtgctggactccgatggatccttcttcctgtactccgacctgaccgtggacaagtctcggtggcagcagggcaacgtgttctcttgcagcgtgatgcacgaggccctgcataatcactacacccaagagtccctgtccctgagtcctggcaag

SEQ ID NO: 185 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVEGFYPSDIAVEWESNGQPENNYKTTPPVLDSDGSFFLYSDLTVDKSRWQQGNVFSCSVMHEALHNHYTQESLSLSPGK

SEQ ID NO: 186 - DNA, 990 bp, synthetic construct
  source 1..990: mol_type = other DNA
gccagcaccaagggcccttctgtctttcctctggcccctagctctaagtccacatctggcggcacagctgctctgggctgcctggtgaaggactacttccccgagcctgtgacagtctcttggaacagcggcgccctgacctccggcgtgcacacctttcctgccgtgcttcaatcctccggcctgtatagcctctcttctgtcgtgaccgtgccatcttcctctctgggcacccagacctacatctgcaacgtgaaccacaagccatccaacaccaaagtggacaaaaaggtggaacccaagtcctgcgacaagacccatacctgtcctccatgccctgctcccgagctgctgggaggcccttccgtgttcctgttccctcctaagcctaaagataccctgatgatctcccggacccctgaggtgacctgcgtggtggtcgatgtgtcccacgaggaccccgaagtgaagttcaactggtacgtggatggcgtggaagtgcacaacgccaagaccaagcctcgggaagagcagtacgcttctacctatagagtggtgtccgtgctgaccgtgctgcaccaggactggctgaacggaaaagagtacaagtgcaaggtctccaacaaggctctgcctgctcctatcgagaagacaatctccaaggccaagggccagcctagagagccccaagtgtacacactgcctccttcccggaagaagctgaccaagaaccaggtcagcctgacctgtctggtgaaaggcttctaccctagcgacatcgccgtggagtgggagtctaatggacagcctgagaacaactataagaccaccccccccgtgctgaagtctgacggctctttcttcctgtactccaagctgacagtggacaaatctcggtggcagcagggcaacgtgttctcctgctccgtgatgcacgaggccctgcataatcactacacccagaagtctctgtctctgtcccctggcaag

SEQ ID NO: 187 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRKKLTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 188 - DNA, 1359 bp, synthetic construct
  source 1..1359: mol_type = other DNA
gaggtgcagctggtggagtccggaggaggactggtgcagcctggaggctccctgaggctgagctgctctgcctccggcttcacattttccagctttggcatgcattgggtgcggcagaccccaggcaagggactggagtgggtggcttatatctcttccggcagctctaccatctactatggcgacacagtgaagggcaggttcaccatcagccgggataacgccaagaactccctgtttctgcagatgtccagcctgagggccgaggacacagccgtgtactattgcgctagagagggcggctactattacggccgcagctattacacaatggactattggggccagggcaccacagtgacagtgtcttccgcctccaccaagggcccaagcgtgttcccactggctcccagctctaagagcacctctggaggaacagccgctctgggatgtctggtgaaggactacttcccagagcccgtgaccgtgtcttggaactccggcgccctgacctccggagtgcacacatttccagctgtgctgcagtccagcggcctgtatagcctgtcttccgtggtgacagtgcccagctcttccctgggcacccagacatacatctgcaacgtgaatcacaagccatccaataccaaggtggacaagaaggtggagcccaagagctgtgataagacccatacatgccccccttgtcctgctccagagctgctgggaggaccatccgtgttcctgtttccacccaagcctaaggacacactgatgatctctagaacccccgaggtgacatgcgtggtggtggacgtgtcccacgaggatcctgaggtgaagtttaactggtacgtggatggcgtggaggtgcataatgctaagaccaagcccagggaggagcagtatggctctacataccgggtggtgtccgtgctgaccgtgctgcaccaggattggctgaacggcaaggagtacaagtgcaaggtgagcaataaggccctgcccgctcctatcgagaagacaatctctaaggccaagggccagcctagagagccacaggtgtataccctgcctccaagccgcgacgagctgacaaagaaccaggtgtctctgacctgtctggtgaagggcttctacccttctgatatcgctgtggagtgggagtccaatggccagccagagaacaattataagaccacaccccctgtgctggacagcgatggctctttctttctgtactccgacctgacagtggataagagccgctggcagcagggcaacgtgttttcctgtagcgtgatgcatgaggccctgcacaatcattacacccaggagtctctgtccctgagccctggc

SEQ ID NO: 189 - protein, 453 aa, synthetic construct
EVQLVESGGGLVQPGGSLRLSCSASGFTFSSFGMHWVRQTPGKGLEWVAYISSGSSTIYYGDTVKGRFTISRDNAKNSLFLQMSSLRAEDTAVYYCAREGGYYYGRSYYTMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSDLTVDKSRWQQGNVFSCSVMHEALHNHYTQESLSLSPG

SEQ ID NO: 190 - DNA, 2109 bp, synthetic construct
  source 1..2109: mol_type = other DNA
gaggtgcagctggtggagtctggaggaggactggtgcagcctggaggctctctgagactgagctgctctgcttccggcttcacattttccagctttggcatgcattgggtgcgccagaccccaggcaagggactggagtgggtggcctacatctcttccggcagctctaccatctactatggcgacacagtgaagggcaggttcaccatctcccgggataacgctaagaatagcctgtttctgcagatgtccagcctgagagctgaggacacagccgtgtactattgcgccagagagggcggctactattacggccgctcctattacacaatggactattggggccagggcaccacagtgacagtgtcttccgcctccaccaagggaccatccgtgttcccactggccccaagctctaagagcacctctggaggaacagccgctctgggatgtctggtgaaggactacttcccagagcccgtgaccgtgtcttggaactccggcgctctgacctctggcgtgcacacatttcccgccgtgctgcagtccagcggcctgtattccctgtcttccgtggtgacagtgcctagctcttccctgggcacccagacatacatctgcaacgtgaatcacaagccctccaataccaaggtggacaagaaggtggagcctaagagctgtgataagacccatacatgccccccttgtcctgctccagagctgctgggaggacctagcgtgttcctgtttccacccaagccaaaggacaccctgatgatctctcgcacccctgaggtgacatgcgtggtggtggacgtgtcccacgaggatccagaggtgaagtttaactggtacgtggatggcgtggaggtgcataatgccaagaccaagcctagggaggagcagtatggcagcacataccgggtggtgtctgtgctgaccgtgctgcaccaggactggctgaacggcaaggagtacaagtgcaaggtgagcaataaggctctgcccgcccctatcgagaagacaatctctaaggctaagggccagcctagggagccacaggtgtataccctgcctccatctcggaaggagctgacaaagaaccaggtgtccctgacctgtctggtgaagggcttctacccatccgatatcgccgtggagtgggagagcaatggccagcccgagaacaattataagaccacaccccctgtgctgaagagcgacggctctttctttctgtactctaagctgacagtggataagtccaggtggcagcagggcaacgtgttttcctgcagcgtgatgcatgaggctctgcacaatcattacacccagaagtctctgtccctgagcccaggcaagggaggaggaggaagcgccgctgccgaggtgcagctgctggaatccggcggcggactggtgcagccaggaggctctctgcggctgtcctgtgctgccagcggcttcacattttccgactatgctatgagctgggtgagacaggctcctggcaagggcctggagtgggtgtccgctatccgctggcacagctcttggacctattacgccgacagcgtgaagggcagattcacaatctctcgcgataactccaagaataccctgtacctgcagatgaacagcctgagggctgaggatacagccgtgtattactgcgccaagatgaagtctacccggcattggatcgacgattggggccagggcaccctggtgacagtgtcctctggaggaggaggatctggaggaggaggctccggcggcggcggctcttccgagctgacccaggaccctgccgtgtccgtggccctgggccagaccgtgagaatcacatgtcagggcgattccctgcgcagctattacgcttcctggtatcagcagaagccaggacaggctcccgtgctggtcatctacggcaagaacaataggccaagcggcatccccgaccggttctctggcagctcttccggcaacaccgcttccctgaccatcacaggagctcaggctgaggacgaggctgattattactgccagagctacgatagctcttcctatcattacgtgtttggcggcggcaccaagctgacagtgctg

SEQ ID NO: 191 - protein, 703 aa, synthetic construct
EVQLVESGGGLVQPGGSLRLSCSASGFTFSSFGMHWVRQTPGKGLEWVAYISSGSSTIYYGDTVKGRFTISRDNAKNSLFLQMSSLRAEDTAVYYCAREGGYYYGRSYYTMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRKELTKNQVSLTCLVKGFYPSDIAVEWESNGQPENNYKTTPPVLKSDGSFFLYSKLTVDKSRWQQGNVFSCSVMHEALHNHYTQKSLSLSPGKGGGGSAAAEVQLLESGGGLVQPGGSLRLSCAASGFTFSDYAMSWVRQAPGKGLEWVSAIRWHSSWTYYADSVKGRFTISRDNSKNTLYLQMNSLRAEDTAVYYCAKMKSTRHWIDDWGQGTLVTVSSGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCQSYDSSSYHYVFGGGTKLTVL

SEQ ID NO: 192 - DNA, 2109 bp, synthetic construct
  source 1..2109: mol_type = other DNA
gaggtgcagctggtggagtctggaggaggactggtgcagcctggaggctccctgagactgtcttgctccgcctccggcttcacattttccagctttggcatgcattgggtgcgccagaccccaggcaagggactggagtgggtggcctacatctcttccggcagctctaccatctactatggcgacacagtgaagggcaggttcaccatctcccgggataacgctaagaatagcctgtttctgcagatgtccagcctgagagctgaggacacagccgtgtactattgcgccagagagggcggctactattacggccgcagctattacacaatggactattggggccagggcaccacagtgacagtgtcttccgcttctaccaagggaccatccgtgttcccactggccccaagctctaagtctacctccggaggaacagccgctctgggatgtctggtgaaggactacttcccagagccagtgaccgtgtcctggaacagcggcgctctgacctccggagtgcacacatttcccgccgtgctgcagtccagcggcctgtatagcctgtcttccgtggtgacagtgcctagctcttccctgggcacccagacatacatctgcaacgtgaatcacaagccctccaataccaaggtggacaagaaggtggagcctaagagctgtgataagacccatacatgccccccttgtcctgctccagagctgctgggaggaccttccgtgttcctgtttccacccaagccaaaggacaccctgatgatctctcgcacccctgaggtgacatgcgtggtggtggacgtgtcccacgaggatccagaggtgaagtttaactggtacgtggatggcgtggaggtgcataatgccaagaccaagcctagggaggagcagtatggctctacataccgggtggtgtccgtgctgaccgtgctgcaccaggactggctgaacggcaaggagtacaagtgcaaggtgagcaataaggctctgcccgcccctatcgagaagacaatctctaaggctaagggccagcctagggagccacaggtgtataccctgcctccatctcggaaggagctgacaaagaaccaggtgtccctgacctgtctggtgaagggcttctacccatccgatatcgccgtggagtgggagagcaatggccagcccgagaacaattataagaccacaccccctgtgctgaagtctgacggctccttctttctgtactctaagctgacagtggataagtccaggtggcagcagggcaacgtgtttagctgctctgtgatgcatgaggctctgcacaatcattacacccagaagtccctgagcctgtctccaggcaagggaggaggaggaagcgccgctgccgaggtgcagctgctggaatccggcggcggactggtgcagccaggaggctccctgcggctgagctgtgctgcctctggcttcaccttctccgactatgctatgtcttgggtgagacaggctcctggcaagggcctggagtgggtgtccgccatcaggtggcactctggctggacctattacgccgacagcgtgaagggcagattcacaatctcccgcgataacagcaagaataccctgtacctgcagatgaactctctgagggctgaggatacagccgtgtattactgcgccaagatgaagtccacccggcattggatcgacgattggggccagggcaccctggtgacagtgagctctggaggaggaggatccggaggaggaggaagcggcggcggcggctccagcgagctgacccaggaccctgccgtgtccgtggccctgggccagaccgtgagaatcacatgtcagggcgatagcctgcgctcttattacgcctcctggtatcagcagaagccaggacaggctcccgtgctggtcatctacggcaagaacaataggccaagcggcatccccgaccggttctctggctcttccagcggcaacaccgcctccctgaccatcacaggagctcaggctgaggacgaggctgattattactgccagtcttacgattcttccagctatcattacgtgtttggcggcggcaccaagctgacagtgctg

SEQ ID NO: 193 - protein, 703 aa, synthetic construct
EVQLVESGGGLVQPGGSLRLSCSASGFTFSSFGMHWVRQTPGKGLEWVAYISSGSSTIYYGDTVKGRFTISRDNAKNSLFLQMSSLRAEDTAVYYCAREGGYYYGRSYYTMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRKELTKNQVSLTCLVKGFYPSDIAVEWESNGQPENNYKTTPPVLKSDGSFFLYSKLTVDKSRWQQGNVFSCSVMHEALHNHYTQKSLSLSPGKGGGGSAAAEVQLLESGGGLVQPGGSLRLSCAASGFTFSDYAMSWVRQAPGKGLEWVSAIRWHSGWTYYADSVKGRFTISRDNSKNTLYLQMNSLRAEDTAVYYCAKMKSTRHWIDDWGQGTLVTVSSGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCQSYDSSSYHYVFGGGTKLTVL

SEQ ID NO: 194 - DNA, 1362 bp, synthetic construct
  source 1..1362: mol_type = other DNA
gaggtgcagctggtcgagagcggcggcggcctggtgcagcctggaggcagcctgcggctgtcttgttctgccagcggcttcacattcagctccttcggcatgcactgggtcagacagacacccggcaaaggcctggaatgggtggcctacatcagcagcggcagctccaccatctactacggcgacaccgttaagggtagattcaccattagccgggacaacgccaagaactccctgtttctgcagatgagcagcctgagagccgaggatacagccgtgtactactgcgccagagaaggcggatactactacggaagaagctactacacaatggactactggggccagggcacaaccgtgacagtgtccagcgctagcactaaaggaccaagcgtgttccctctggctccaagcagcaaatcaacctcaggcggcacagcagcactggggtgtctggtgaaggactacttcccagagcccgtcaccgtgtcatggaacagcggagcactgactagcggagtccacacctttccagccgtgctgcagagctccggactgtactccctgtctagtgtggtcacagtgccttcaagctccctggggactcagacctatatctgcaacgtgaatcacaagccctccaatactaaggtcgacaaacgagtggagcctaagtcttgtgataaaacacatacttgccccccttgtcctgcaccagaactgctgggaggacctagcgtgttcctgtttccacccaagccaaaagacaccctgatgattagtagaacccctgaggtcacatgcgtggtcgtggacgtgagccacgaggatccagaagtcaagttcaactggtacgtggatggcgtcgaagtgcataatgctaagacaaaaccccgggaggaacagtacaacagtacctatagagtcgtgtcagtcctgacagtgctgcatcaggactggctgaacgggaaagagtataagtgcaaagtgtccaataaggccctgcccgctcctatcgagaaaactatttctaaggctaaaggccagccaagggaaccccaggtgtacaccctgcctccatcacgcgaggaaatgacaaagaaccaggtcagcctgacttgtctggtgaaagggttctatccatctgacatcgcagtggagtgggaaagtaatggacagcccgaaaacaattacaagaccacaccccctgtgctggactccgatggatctttctttctgtatagcaagctgaccgtggataaatcccggtggcagcagggcaatgtcttttcttgtagtgtgatgcacgaggcactgcacaaccactacacccagaagtcactgtccctgagccccggaaag

SEQ ID NO: 195 - protein, 454 aa, synthetic construct
EVQLVESGGGLVQPGGSLRLSCSASGFTFSSFGMHWVRQTPGKGLEWVAYISSGSSTIYYGDTVKGRFTISRDNAKNSLFLQMSSLRAEDTAVYYCAREGGYYYGRSYYTMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 196 - DNA, 657 bp, synthetic construct
  source 1..657: mol_type = other DNA
gatgtggtgatgactcaaagtccactaagtctgcctgctacccctggcgatcctgcctctatctcctgccggtcctctcagtccatcgtgcactctaacggtaatacatatctggaatggtacctgcagaaaccaggccagtctcctaagctgctgatctacaaggtgtccaaccggttttctggcgtgcccgatagattttccggcagcggctctggaaccgacttcaccctcaccatctctagagtggatgccgaggacgtgggaatctactactgcttccagggcagtcatgtgcctcctaccttcggccctggcacaaagctggagatcaagcggaccgtggccgctccatccgtcttcatcttccccccctccgacgaacagctgaagtccggcaccgcttctgtggtgtgcctgctgaacaacttctaccctagagaagccaaagtgcagtggaaggtggacaacgctctgcagagcggcaactcccaagagagcgtcaccgagcaggactccaaggactctacctactccctgtccagcaccctgacactgtctaaggccgactacgagaagcacaaggtgtacgcctgcgaggtgacccaccagggcctgagctcccctgtgaccaagtccttcaacagaggcgagtgt

SEQ ID NO: 197 - protein, 219 aa, synthetic construct
DVVMTQSPLSLPATPGDPASISCRSSQSIVHSNGNTYLEWYLQKPGQSPKLLIYKVSNRFSGVPDRFSGSGSGTDFTLTISRVDAEDVGIYYCFQGSHVPPTFGPGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 198 - DNA, 1362 bp, synthetic construct
  source 1..1362: mol_type = other DNA
gaagtgcaactagtggaaagtggtggtggtctggtgcagcctggaggcagcctgcggctgtcctgctccgcctccggcttcaccttctccagcttcggcatgcactgggtccggcagacccctggcaagggcctggaatgggtggcctacatctcctctggctccagcaccatctactatggcgatacagtgaagggcagattcacaatttctcgcgacaacgccaagaactctctgttcctgcagatgtcaagcctcagagccgaggataccgctgtgtactactgcgccagagaaggcggctactactacggcagatcctactacacaatggactactggggccagggcaccacggtgaccgtgagctccgctaagaccaccgccccctccgtgtatcctctggcccctgtatgcggcgacaccactggctcttctgtgaccctgggctgcctggtcaaaggctacttccccgagcctgtgacactgacctggaactccggctccctgtcctccggcgtgcacacctttcccgccgtgctgcaatctgatctgtacaccttgtcttcctctgtgaccgtgacctcctctacctggccttctcagtccatcacctgcaacgtggctcatcccgcttcttctaccaaagtggacaagaagatcgagcctagaggccctacaatcaagccttgtccaccttgcaagtgtcctgctcctaacctgctgggaggaccttccgtgttcatcttccctcctaagatcaaggacgtgctgatgatctctctgtctcccatcgtgacatgcgtggtggtggatgtgtccgaggacgaccccgacgtgcagatctcctggttcgtgaataatgtggaagtgcataccgcccagacccagacccaccgggaagattacaacagcacactgagagtcgtgtctgccctgcctatccagcaccaggactggatgtccggaaaagagttcaagtgcaaggtgaacaacaaggacctgcctgctccaatcgaacggaccatctccaagcccaagggctccgtgcgggctcctcaggtgtacgtgctgcctcctccagaggaagagatgaccaagaagcaagtgaccctgacctgcatggtgaccgacttcatgcctgaggacatctacgtggagtggaccaacaacggcaagacagagctgaactacaagaacaccgaacccgtactggactccgatggttcttactttatgtactccgacctgagagtggagaagaaaaactgggtcgagagaaactcctactcttgtagcgtcgttcacgagggcctgcacaaccaccacaccaccgagtccttttctagaacacctggcaaa

SEQ ID NO: 199 - protein, 454 aa, synthetic construct
EVQLVESGGGLVQPGGSLRLSCSASGFTFSSFGMHWVRQTPGKGLEWVAYISSGSSTIYYGDTVKGRFTISRDNAKNSLFLQMSSLRAEDTAVYYCAREGGYYYGRSYYTMDYWGQGTTVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSDLRVEKKNWVERNSYSCSVVHEGLHNHHTTESFSRTPGK

SEQ ID NO: 200 - DNA, 2109 bp, synthetic construct
  source 1..2109: mol_type = other DNA
gaagtgcaactagtggaaagtggtggtggtctggtgcagcctggaggatccctgcggctgtcctgctccgcttctggcttcaccttcagctccttcggcatgcactgggtgcggcagacccctggcaaaggcctggaatgggtggcctacatctcttctggcagtagcaccatctactatggcgacaccgtgaaaggacgctttaccatctccagagataacgccaagaactccctgttcctgcagatgtcttctctgcgggccgaggacacagccgtgtactactgtgccagagagggcggctactactacggcagatcctattacacaatggactactggggacagggcaccaccgtgaccgttagctctgccaagaccaccgctccatctgtgtatcctctggcccccgtgtgcggcgacaccaccggctcctccgtgaccctgggctgcctggtgaagggctacttcccagaacctgtgacactgacctggaactccggctccctgtcctccggcgtgcataccttccccgctgtgctgcagtctgatctctatacactctcctcctccgtgaccgtgacaagctccacctggccttcccagtccatcacctgcaatgtggctcatcctgcatcgtctaccaaggtggacaagaaaatcgagcctagaggccctactatcaagccttgtcctccttgcaagtgccctgctcccaacctgctgggaggcccttccgtcttcatcttccctcctaagatcaaggacgtgctgatgatcagcctgtctccaatcgtgacctgtgtggtggtggacgtgtctgaggacgaccctgatgtgcagatctcctggttcgtgaataatgtggaagtgcataccgctcagacccagacacaccgggaagattacaactcaaccctgagagtggtgagcgctctgccaatccagcaccaggactggatgtccggaaaggagttcaagtgtaaagtgaacaacaaggacctgcccgctcccatcgagcggactatttctaagcccaagggctctgttagagcccctcaagtgtacgtcctgcctccacctgagaaagagatgaccaagaagcaggtgaccctgacctgcatggtgaccgacttcatgcctgaggacatctacgtggaatggaccaacaacggcaaaaccgaactgaactacaagaacaccgagcccgtgctgaagagcgacggctcttacttcatgtactccaagctgagggtcgagaagaaaaactgggtcgaacggaactcttactcttgttctgtggtgcacgagggactgcacaaccaccacaccaccaaatctttctcccggacacctggcaagggcggaggtggctctgccgctgctgaggtgcagctgctggagtccggtggtggcctggtccagcccggcggctctctgagactgtcctgcgccgcctccggatttaccttttcggactacgctatgtcctgggtcagacaagctcctggcaagtgcctggaatgggtctccgccatcagatggcactctggctggacctactacgctgattccgtgaagggcagattcacaatctctcgggacaactccaagaatacactgtacctgcagatgaacagcctcagagccgaggacaccgccgtgtactactgcgctaagatgaagtccaccagacactggatcgacgattggggccaaggcacactggtgaccgtgtcttccggaggcggtggatctggtggtggcggatcaggtggaggcgggagctccgagctgacacaggatcctgctgtgtccgtggccctgggccagaccgtgagaatcacctgccagggcgattctctgcggtcctactacgcctcttggtaccagcagaagcccggccaggcccctgtgctggtgatctacggcaagaacaacagaccttccggcatccctgaccggttctccggtagctcttctggcaacaccgcctctctcaccatcaccggcgcccaggccgaagacgaggccgactactactgccagtcttacgactctagttcctaccactacgtgtttggatgtgggaccaagctgaccgtgttg

SEQ ID NO: 201 - protein, 703 aa, synthetic construct
EVQLVESGGGLVQPGGSLRLSCSASGFTFSSFGMHWVRQTPGKGLEWVAYISSGSSTIYYGDTVKGRFTISRDNAKNSLFLQMSSLRAEDTAVYYCAREGGYYYGRSYYTMDYWGQGTTVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEKEMTKKQVTLTCMVTDFMPEDIYVEWTNNGKTELNYKNTEPVLKSDGSYFMYSKLRVEKKNWVERNSYSCSVVHEGLHNHHTTKSFSRTPGKGGGGSAAAEVQLLESGGGLVQPGGSLRLSCAASGFTFSDYAMSWVRQAPGKCLEWVSAIRWHSGWTYYADSVKGRFTISRDNSKNTLYLQMNSLRAEDTAVYYCAKMKSTRHWIDDWGQGTLVTVSSGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCQSYDSSSYHYVFGCGTKLTVL

SEQ ID NO: 202 - DNA, 990 bp, synthetic construct
  source 1..990: mol_type = other DNA
gcctctaccaagggccctagcgtgttccccctggccccttcttctaagtccacctccggcggcaccgctgctctcggctgcctggtgaaggactacttccccgagcctgtcaccgtgtcctggaactccggcgccctgacaagcggcgtgcacaccttccccgctgtgctgcagtctagcggcctgtattctctgtcctctgtggtgaccgtgccttcctccagcctgggcacccagacctacatctgcaacgtgaaccacaagccctccaatacaaaagtggacaagcgggtggaacccaagtcctgcgacaaaactcatacctgtcctccttgccctgctcctgagctgctgggcggaccttctgtgtttctgtttcctcctaaacccaaggacaccctgatgatctcccggacccccgaagtgacctgcgtggtcgtggacgtgtctcacgaagatcctgaggtcaagttcaactggtacgtggatggagtggaagtgcataacgccaagaccaagcctagagaagagcagtacgcctccacctacagagtggtgtctgtgctgacagtgctgcaccaggactggctgaacggcaaagagtacaagtgcaaggtgtccaacaaggctctgcctgctcctatcgagaagaccatcagtaaggccaaaggccagccccgcgaacctcaggtgtacacactgcctccatccagagaggagatgaccaagaatcaagtgtccctgacctgtctggtgaagggattctacccttctgacatcgccgttgagtgggagtccaacggccagcctgagaacaactacaagaccaccccacctgtgctggactccgacggctcttttttcctgtactccaagctgaccgtggacaagagcagatggcagcagggcaacgtgttctcctgctccgtgatgcacgaggctctgcacaatcactacacccagaagtctctgtccctgtctcctggcaag

SEQ ID NO: 203 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 204 - DNA, 1737 bp, synthetic construct
  source 1..1737: mol_type = other DNA
gccagcaccaagggccctagcgtgttccctctggccccttcgtccaagtctacctccggcggcactgccgccctgggctgtctggtgaaggactacttccctgaacccgtcaccgtgtcctggaactccggcgccctgacctctggcgtgcacacctttcccgccgtgctgcagtcttctggactgtactccctgtcctccgtcgtgaccgtaccttcctcctccctgggaacccagacctacatctgcaacgtgaatcacaagcccagcaataccaaagtggataagaaagtggaacctaagtcctgcgacaaaacccacacatgtccaccctgtcctgctccagagctgctgggcggcccatccgtgttcctgttccctccaaaacccaaggacaccctgatgatctccagaacccccgaggtgacctgcgtggtggtggacgtgtcccacgaggaccctgaggtcaagttcaactggtacgtggacggcgtcgaagtgcataacgccaagaccaagcctcgggaggaacagtacgccagcacctacagagtggtgtccgtgttgacagtgctgcaccaggattggctgaacggcaaagaatacaagtgcaaggtgtccaacaaggccctgcccgcccctatcgagaagaccatctctaaggccaagggccagcctagagagcctcaggtgtacaccctgcccccttccagagatgagcttaccaagaaccaggtctctctgacctgcctggtgaaaggcttctacccttccgacatcgccgtagaatgggagtccaatggccagcctgagaacaactacaagaccacccctcctgtgctggactccgacggctctttctttctgtacagtaagctgaccgtggacaagtctagatggcagcagggcaacgtgttcagctgctccgtgatgcacgaagctctgcacaaccattacacacagaagagcctgtctctgtcccctggcaagggcggaggtggctctgccgctgcagaggtgcagctgctggaatctggcggtggcctggttcagcccggcggctccctgagactgtcctgcgctgcttctggcttcaccttctctgactatgccatgtcttgggtgcgccaggctccaggcaagggcctggagtgggtttccgccatccggtggcactcctcctggacctattacgccgactccgtgaagggcagatttacaattagccgggacaactccaagaacaccctgtacctgcagatgaacagcctgagagccgaggataccgctgtgtactactgcgctaagatgaagtccaccagacactggatcgacgactggggccagggcaccctggtgacagtctcctccggaggcggtggatctggtggtggcggatcaggtggaggcgggagctccgagctgacccaagatcctgctgtgtctgtggctctcggccaaactgtgagaatcacctgccaaggcgattccctgcggtcctactacgcctcttggtaccagcagaagcctggacaagctcctgtgctggtgatctacggcaagaacaaccggccctctggcatccctgaccggttcagtggatcctcttccggcaacactgcctctctgaccatcaccggcgctcaggccgaggacgaggccgactactactgtcagagctacgactcctcatcctaccactacgtgttcggcggcggcaccaagctgaccgtcctg

SEQ ID NO: 205 - protein, 579 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSAAAEVQLLESGGGLVQPGGSLRLSCAASGFTFSDYAMSWVRQAPGKGLEWVSAIRWHSSWTYYADSVKGRFTISRDNSKNTLYLQMNSLRAEDTAVYYCAKMKSTRHWIDDWGQGTLVTVSSGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCQSYDSSSYHYVFGGGTKLTVL

SEQ ID NO: 206 - DNA, 1737 bp, synthetic construct
  source 1..1737: mol_type = other DNA
gccagcaccaagggccctagcgtgttccctctggccccttcgtccaagtctacctccggcggcactgccgccctgggctgtctggtgaaggactacttccctgaacccgtcaccgtgtcctggaactccggcgccctgacctctggcgtgcacacctttcccgccgtgctgcagtcttctggactgtactccctgtcctccgtcgtgaccgtaccttcctcctccctgggaacccagacctacatctgcaacgtgaatcacaagcccagcaataccaaagtggataagaaagtggaacctaagtcctgcgacaaaacccacacatgtccaccctgtcctgctccagagctgctgggcggcccatccgtgttcctgttccctccaaaacccaaggacaccctgatgatctccagaacccccgaggtgacctgcgtggtggtggacgtgtcccacgaggaccctgaggtcaagttcaactggtacgtggacggcgtcgaagtgcataacgccaagaccaagcctcgggaggaacagtacgccagcacctacagagtggtgtccgtgttgacagtgctgcaccaggattggctgaacggcaaagaatacaagtgcaaggtgtccaacaaggccctgcccgcccctatcgagaagaccatctctaaggccaagggccagcctagagagcctcaggtgtacaccctgcccccttccagagatgagcttaccaagaaccaggtctctctgacctgcctggtgaaaggcttctacccttccgacatcgccgtagaatgggagtccaatggccagcctgagaacaactacaagaccacccctcctgtgctggactccgacggctctttctttctgtacagtaagctgaccgtggacaagtctagatggcagcagggcaacgtgttcagctgctccgtgatgcacgaagctctgcacaaccattacacacagaagagcctgtctctgtcccctggcaagggcggaggtggctctgccgctgcagaggtgcagctgctggaatctggcggtggcctggttcagcccggcggctccctgagactgtcctgcgctgcttctggcttcaccttctctgactatgccatgtcttgggtgcgccaggctccaggcaagggcctggagtgggtttccgccatccggtggcactccggctggacctattacgccgactccgtgaagggcagatttacaattagccgggacaactccaagaacaccctgtacctgcagatgaacagcctgagagccgaggataccgctgtgtactactgcgctaagatgaagtccaccagacactggatcgacgactggggccagggcaccctggtgacagtctcctccggaggcggtggatctggtggtggcggatcaggtggaggcgggagctccgagctgacccaagatcctgctgtgtctgtggctctcggccaaactgtgagaatcacctgccaaggcgattccctgcggtcctactacgcctcttggtaccagcagaagcctggacaagctcctgtgctggtgatctacggcaagaacaaccggccctctggcatccctgaccggttcagtggatcctcttccggcaacactgcctctctgaccatcaccggcgctcaggccgaggacgaggccgactactactgtcagagctacgactcctcatcctaccactacgtgttcggcggcggcaccaagctgaccgtcctg

SEQ ID NO: 207 - protein, 579 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSAAAEVQLLESGGGLVQPGGSLRLSCAASGFTFSDYAMSWVRQAPGKGLEWVSAIRWHSGWTYYADSVKGRFTISRDNSKNTLYLQMNSLRAEDTAVYYCAKMKSTRHWIDDWGQGTLVTVSSGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCQSYDSSSYHYVFGGGTKLTVL

SEQ ID NO: 208 - DNA, 987 bp, synthetic construct
  source 1..987: mol_type = other DNA
gcctctaccaagggcccctctgtgttccctctggccccttcgtctaagtccacctctggcggcaccgctgctctgggctgcctggttaaggactacttcccagagccagtgacagtgagctggaactccggtgctctgacctccggcgtgcataccttccctgccgtgctgcagtcctctggcctgtactccctgtccagcgtggtgaccgtgcctagctcctccctgggcacacagacctacatctgcaacgtgaaccataagccttccaacaccaaagtggataagaaggtggaacccaagtcctgcgacaagacccacacctgcccaccttgtcctgcacctgaactgctggggggcccctccgtctttctgtttcctccaaaacctaaggataccctgatgatcagccggacacctgaagtgacctgcgtggtcgtcgacgtgtcccacgaagaccccgaggtgaagttcaactggtacgtggacggcgtggaagtgcacaacgccaagaccaagcccagagaagagcagtacgcctctacctacagagtggtgtccgtgctgaccgttctgcaccaggactggctgaacggcaaagaatacaagtgcaaggtgtccaacaaggctctgcctgcccctatcgagaagaccatcagcaaggccaagggacagcctagagagcctcaagtgtacactctgcctccttctcgcgatgagctgacaaagaaccaggtgtccctgacctgtctcgtgaaaggcttctacccttctgacatcgccgtcgagtgggagtctaatggacagcccgagaacaactacaagaccacccctcccgtgctggactccgacggatctttcttcctgtattccaagctgacagtcgacaagtccagatggcagcagggcaacgtgttcagctgctccgtgatgcacgaggccctgcacaatcactacacccagaagagcttgtctctgtctcctggc

SEQ ID NO: 209 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 210 - DNA, 1068 bp, synthetic construct
  source 1..1068: mol_type = other DNA
cggaccgtggctgccccatctgtgttcatcttccctccttccgacgaacagctgaagtccggcaccgcctccgtggtgtgcctgctgaacaacttctaccccagagaggccaaggtgcagtggaaggtggacaatgctctgcagtccggcaactcccaagagtctgtcaccgagcaggacagtaaggactccacctactccctaagttctaccctcaccctgtccaaggccgactacgagaagcacaaggtgtacgcttgcgaggtgacccaccagggcctgtcttcccctgtgaccaagtccttcaaccggggcgagtgcggcggcggaggctctgccgctgctgaagtgcagctgctcgaatctggcggcggactggtgcagcctggcggctccctgagactgtcttgtgccgcttctggcttcaccttctccgattacgccatgtcctgggtgagacaggcccctggcaagggcctggaatgggtctccgccatcagatggcattcttcttggacctactacgccgattccgtgaagggcagattcaccatctccagagacaactcgaagaacaccctgtatctgcaaatgaactctctgagggccgaggacaccgctgtgtactactgtgctaagatgaaatctacaagacactggatcgacgactggggccagggaacactggtgaccgtgtccagcggaggcggtggctctggaggaggcggcagcggcgggggcggatcttccgagctgacccaggatcctgccgtgtctgtggccctgggacagaccgtgcggatcacctgccagggcgactccctgagatcctactacgcctcatggtaccagcagaaaccaggccaggctcctgtgctggtgatctacggcaagaacaacagaccttctggcatccctgatagattttccggcagctcctctggtaataccgcttctctgacaatcaccggcgctcaggccgaggacgaggccgactactactgccagtcttacgactctagcagctaccactacgtgtttggcggcggcaccaagctgaccgtgctc

SEQ ID NO: 211 - protein, 356 aa, synthetic construct
RTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSAAAEVQLLESGGGLVQPGGSLRLSCAASGFTFSDYAMSWVRQAPGKGLEWVSAIRWHSSWTYYADSVKGRFTISRDNSKNTLYLQMNSLRAEDTAVYYCAKMKSTRHWIDDWGQGTLVTVSSGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCQSYDSSSYHYVFGGGTKLTVL

SEQ ID NO: 212 - DNA, 1068 bp, synthetic construct
  source 1..1068: mol_type = other DNA
cggaccgtggctgccccatctgtgttcatcttccctccttccgacgaacagctgaagtccggcaccgcctccgtggtgtgcctgctgaacaacttctaccccagagaggccaaggtgcagtggaaggtggacaatgctctgcagtccggcaactcccaagagtctgtcaccgagcaggacagtaaggactccacctactccctaagttctaccctcaccctgtccaaggccgactacgagaagcacaaggtgtacgcttgcgaggtgacccaccagggcctgtcttcccctgtgaccaagtccttcaaccggggcgagtgcggcggcggaggctctgccgctgctgaagtgcagctgctcgaatctggcggcggactggtgcagcctggcggctccctgagactgtcttgtgccgcttctggcttcaccttctccgattacgccatgtcctgggtgagacaggcccctggcaagggcctggaatgggtctccgccatcagatggcattctggctggacctactacgccgattccgtgaagggcagattcaccatctccagagacaactcgaagaacaccctgtatctgcaaatgaactctctgagggccgaggacaccgctgtgtactactgtgctaagatgaaatctacaagacactggatcgacgactggggccagggaacactggtgaccgtgtccagcggaggcggtggctctggaggaggcggcagcggcgggggcggatcttccgagctgacccaggatcctgccgtgtctgtggccctgggacagaccgtgcggatcacctgccagggcgactccctgagatcctactacgcctcatggtaccagcagaaaccaggccaggctcctgtgctggtgatctacggcaagaacaacagaccttctggcatccctgatagattttccggcagctcctctggtaataccgcttctctgacaatcaccggcgctcaggccgaggacgaggccgactactactgccagtcttacgactctagcagctaccactacgtgtttggcggcggcaccaagctgaccgtgctc

SEQ ID NO: 213 - protein, 356 aa, synthetic construct
RTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSAAAEVQLLESGGGLVQPGGSLRLSCAASGFTFSDYAMSWVRQAPGKGLEWVSAIRWHSGWTYYADSVKGRFTISRDNSKNTLYLQMNSLRAEDTAVYYCAKMKSTRHWIDDWGQGTLVTVSSGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCQSYDSSSYHYVFGGGTKLTVL

SEQ ID NO: 214 - DNA, 1737 bp, synthetic construct
  source 1..1737: mol_type = other DNA
gcttccaccaagggccctagcgtgtttcctctggcccctagctccaagagtacctccggcggcaccgcagctctgggctgcctcgtgaaggactacttccctgaacccgtgacagtgtcttggaactcgggcgctctgaccagtggagtgcacacctttccagccgtgctgcagtcttccggcctgtactccctgagctctgtggtgacagtgccttctagctctctgggcacacagacctacatctgcaacgtgaaccacaaaccctccaacacgaaagtggacaagaaggtggaacctaagtcctgcgacaagacccatacctgccctccttgccctgctcctgagctgctgggcgggccttccgtgtttctgttcccccctaaacccaaggacaccctaatgatctccagaacccctgaagttacatgcgtggtcgtggacgtgtcccacgaggatccagaggtgaagttcaactggtatgtggatggcgtggaagtgcacaacgccaagaccaagccaagagaagagcagtacgcgtccacctacagagtggtgtccgtgctcaccgtgctgcaccaggactggctgaacggcaaagagtacaagtgcaaggtgtccaacaaggccctgccagctcctatcgagaagaccatctctaaggctaagggccaacctagagagcctcaggtgtacaccctgcctccctctcgggacgagctgacaaagaaccaggtctccctgacctgcctggtcaaaggcttctacccctccgacatcgccgtcgagtgggagtccaatggccagcccgagaacaactacaagacaacacctcctgtgctggactccgacggctccttcttcctgtactccaagctgaccgtggataaatctcggtggcagcagggcaacgtgttctcctgctccgtgatgcacgaggctctgcataatcactacacccaaaagtctctgagcctgtctcctggaaagggcggaggcggatccgccgctgctgaagtgcagctgctggagtctggcggaggcctggtgcagcctgggggctcactgcggctgtcttgcgccgctagcggctttaccttcagcgattacgccatgtcctgggtgcggcaggccccaggcaagtgcctggaatgggtgtctgccatcagatggcactcctcctggacctactacgctgattctgtgaaaggccggttcaccatttcccgggataatagcaagaataccctgtacctgcagatgaactctctgcgggctgaggacaccgccgtgtactactgcgccaagatgaagtctaccagacactggatcgacgactggggccaaggcaccctggtcaccgtgtcctccggcggcggaggatccggtggcggcgggtctggcggaggcggctcttccgaactgacccaggatcctgccgtgtccgtggccctgggacagacagtacggatcacctgtcagggcgactccctgcgctcttactacgcctcttggtaccagcagaagcctggccaggcccctgtgctggtgatctatggcaagaacaacagaccttctggcatccctgacagattctccggaagctccagcggcaacaccgcttctctcaccatcaccggcgctcaggccgaagatgaggccgactactactgccagtcctacgactcttcctcctaccactacgtgttcggctgtggcaccaagctgaccgtgctg

SEQ ID NO: 215 - protein, 579 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSAAAEVQLLESGGGLVQPGGSLRLSCAASGFTFSDYAMSWVRQAPGKCLEWVSAIRWHSSWTYYADSVKGRFTISRDNSKNTLYLQMNSLRAEDTAVYYCAKMKSTRHWIDDWGQGTLVTVSSGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCQSYDSSSYHYVFGCGTKLTVL

SEQ ID NO: 216 - DNA, 1737 bp, synthetic construct
  source 1..1737: mol_type = other DNA
gcttccaccaagggccctagcgtgtttcctctggcccctagctccaagagtacctccggcggcaccgcagctctgggctgcctcgtgaaggactacttccctgaacccgtgacagtgtcttggaactcgggcgctctgaccagtggagtgcacacctttccagccgtgctgcagtcttccggcctgtactccctgagctctgtggtgacagtgccttctagctctctgggcacacagacctacatctgcaacgtgaaccacaaaccctccaacacgaaagtggacaagaaggtggaacctaagtcctgcgacaagacccatacctgccctccttgccctgctcctgagctgctgggcgggccttccgtgtttctgttcccccctaaacccaaggacaccctaatgatctccagaacccctgaagttacatgcgtggtcgtggacgtgtcccacgaggatccagaggtgaagttcaactggtatgtggatggcgtggaagtgcacaacgccaagaccaagccaagagaagagcagtacgcgtccacctacagagtggtgtccgtgctcaccgtgctgcaccaggactggctgaacggcaaagagtacaagtgcaaggtgtccaacaaggccctgccagctcctatcgagaagaccatctctaaggctaagggccaacctagagagcctcaggtgtacaccctgcctccctctcgggacgagctgacaaagaaccaggtctccctgacctgcctggtcaaaggcttctacccctccgacatcgccgtcgagtgggagtccaatggccagcccgagaacaactacaagacaacacctcctgtgctggactccgacggctccttcttcctgtactccaagctgaccgtggataaatctcggtggcagcagggcaacgtgttctcctgctccgtgatgcacgaggctctgcataatcactacacccaaaagtctctgagcctgtctcctggaaagggcggaggcggatccgccgctgctgaagtgcagctgctggagtctggcggaggcctggtgcagcctgggggctcactgcggctgtcttgcgccgctagcggctttaccttcagcgattacgccatgtcctgggtgcggcaggccccaggcaagtgcctggaatgggtgtctgccatcagatggcactccggctggacctactacgctgattctgtgaaaggccggttcaccatttcccgggataatagcaagaataccctgtacctgcagatgaactctctgcgggctgaggacaccgccgtgtactactgcgccaagatgaagtctaccagacactggatcgacgactggggccaaggcaccctggtcaccgtgtcctccggcggcggaggatccggtggcggcgggtctggcggaggcggctcttccgaactgacccaggatcctgccgtgtccgtggccctgggacagacagtacggatcacctgtcagggcgactccctgcgctcttactacgcctcttggtaccagcagaagcctggccaggcccctgtgctggtgatctatggcaagaacaacagaccttctggcatccctgacagattctccggaagctccagcggcaacaccgcttctctcaccatcaccggcgctcaggccgaagatgaggccgactactactgccagtcctacgactcttcctcctaccactacgtgttcggctgtggcaccaagctgaccgtgctg

SEQ ID NO: 217 - protein, 579 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSAAAEVQLLESGGGLVQPGGSLRLSCAASGFTFSDYAMSWVRQAPGKCLEWVSAIRWHSGWTYYADSVKGRFTISRDNSKNTLYLQMNSLRAEDTAVYYCAKMKSTRHWIDDWGQGTLVTVSSGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCQSYDSSSYHYVFGCGTKLTVL

SEQ ID NO: 218 - DNA, 1068 bp, synthetic construct
  source 1..1068: mol_type = other DNA
cggaccgtggccgctccctctgtgttcatcttccctccttccgacgaacagctgaaatcgggaaccgcttccgtggtgtgcctgctgaacaacttctaccctagagaggccaaggttcagtggaaggtcgacaacgccctgcaatctggaaattctcaagagtccgtgaccgagcaggactctaaggactctacatactccctgagttccacactgactctgtccaaggccgattacgagaagcacaaagtgtatgcatgcgaggtgacccaccagggactctcttccccagtgaccaagtccttcaacagaggcgagtgcggcggaggtggctctgctgccgccgaagtgcagctgctggaatctggcggaggactggtgcagcccggcggcagcctgagactgtcctgcgctgcttctggcttcaccttctctgactacgccatgtcctgggtccggcaggcccctggcaagtgcctggaatgggtgagcgccatcagatggcatagctcctggacatattacgctgattccgtgaagggcaggtttaccatctccagagataactccaagaataccctgtacctgcagatgaacagcctaagagccgaggacaccgctgtgtactactgtgctaagatgaagagcaccagacactggatcgacgactggggccagggcaccctggtcaccgtgtcctccggaggcggtggatctggtggtggcggatcaggtggaggcgggagctccgagctgacccaggatcctgctgtctccgtggctctgggacagaccgtgcggatcacctgccagggcgactccctgcggtcctactacgcctcttggtaccaacagaaacccggccaggcccccgtgctggtgatctacggcaagaacaaccggccttccggcatccccgaccggttcagcggctccagctcaggcaacaccgcctccctgacaatcaccggcgctcaagctgaggacgaggccgactactactgtcaatcctacgactccagctcttatcactacgtgtttggctgtggcaccaagctgaccgtgctc

SEQ ID NO: 219 - protein, 356 aa, synthetic construct
RTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSAAAEVQLLESGGGLVQPGGSLRLSCAASGFTFSDYAMSWVRQAPGKCLEWVSAIRWHSSWTYYADSVKGRFTISRDNSKNTLYLQMNSLRAEDTAVYYCAKMKSTRHWIDDWGQGTLVTVSSGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCQSYDSSSYHYVFGCGTKLTVL

SEQ ID NO: 220 - DNA, 1068 bp, synthetic construct
  source 1..1068: mol_type = other DNA
cggaccgtggccgctccctctgtgttcatcttccctccttccgacgaacagctgaaatcgggaaccgcttccgtggtgtgcctgctgaacaacttctaccctagagaggccaaggttcagtggaaggtcgacaacgccctgcaatctggaaattctcaagagtccgtgaccgagcaggactctaaggactctacatactccctgagttccacactgactctgtccaaggccgattacgagaagcacaaagtgtatgcatgcgaggtgacccaccagggactctcttccccagtgaccaagtccttcaacagaggcgagtgcggcggaggtggctctgctgccgccgaagtgcagctgctggaatctggcggaggactggtgcagcccggcggcagcctgagactgtcctgcgctgcttctggcttcaccttctctgactacgccatgtcctgggtccggcaggcccctggcaagtgcctggaatgggtgagcgccatcagatggcatagcggctggacatattacgctgattccgtgaagggcaggtttaccatctccagagataactccaagaataccctgtacctgcagatgaacagcctaagagccgaggacaccgctgtgtactactgtgctaagatgaagagcaccagacactggatcgacgactggggccagggcaccctggtcaccgtgtcctccggaggcggtggatctggtggtggcggatcaggtggaggcgggagctccgagctgacccaggatcctgctgtctccgtggctctgggacagaccgtgcggatcacctgccagggcgactccctgcggtcctactacgcctcttggtaccaacagaaacccggccaggcccccgtgctggtgatctacggcaagaacaaccggccttccggcatccccgaccggttcagcggctccagctcaggcaacaccgcctccctgacaatcaccggcgctcaagctgaggacgaggccgactactactgtcaatcctacgactccagctcttatcactacgtgtttggctgtggcaccaagctgaccgtgctc

SEQ ID NO: 221 - protein, 356 aa, synthetic construct
RTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSAAAEVQLLESGGGLVQPGGSLRLSCAASGFTFSDYAMSWVRQAPGKCLEWVSAIRWHSGWTYYADSVKGRFTISRDNSKNTLYLQMNSLRAEDTAVYYCAKMKSTRHWIDDWGQGTLVTVSSGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCQSYDSSSYHYVFGCGTKLTVL

SEQ ID NO: 222 - DNA, 1737 bp, synthetic construct
  source 1..1737: mol_type = other DNA
gcttccaccaagggccctagcgtgtttcctctggcccctagctccaagagtacctccggcggcaccgcagctctgggctgcctcgtgaaggactacttccctgaacccgtgacagtgtcttggaactcgggcgctctgaccagtggagtgcacacctttccagccgtgctgcagtcttccggcctgtactccctgagctctgtggtgacagtgccttctagctctctgggcacacagacctacatctgcaacgtgaaccacaaaccctccaacacgaaagtggacaagaaggtggaacctaagtcctgcgacaagacccatacctgccctccttgccctgctcctgagctgctgggcgggccttccgtgtttctgttcccccctaaacccaaggacaccctaatgatctccagaacccctgaagttacatgcgtggtcgtggacgtgtcccacgaggatccagaggtgaagttcaactggtatgtggatggcgtggaagtgcacaacgccaagaccaagccaagagaagagcagtacggctccacctacagagtggtgtccgtgctcaccgtgctgcaccaggactggctgaacggcaaagagtacaagtgcaaggtgtccaacaaggccctgccagctcctatcgagaagaccatctctaaggctaagggccaacctagagagcctcaggtgtacaccctgcctccctctcgggacgagctgacaaagaaccaggtctccctgacctgcctggtcaaaggcttctacccctccgacatcgccgtcgagtgggagtccaatggccagcccgagaacaactacaagacaacacctcctgtgctggactccgacggctccttcttcctgtactccaagctgaccgtggataaatctcggtggcagcagggcaacgtgttctcctgctccgtgatgcacgaggctctgcataatcactacacccaaaagtctctgagcctgtctcctggaaagggcggaggcggatccgccgctgctgaagtgcagctgctggagtctggcggaggcctggtgcagcctgggggctcactgcggctgtcttgcgccgccagcggctttaccttcagcgattacgccatgtcctgggtgcggcaggccccaggcaagggcctggaatgggtgtctgccatcagatggcactcctcctggacctactacgctgattctgtgaaaggccggttcaccatttcccgggataatagcaagaataccctgtacctgcagatgaactctctgcgggctgaggacaccgccgtgtactactgcgccaagatgaagtctaccagacacagcatcgacgactggggccaaggcaccctggtcaccgtgtcctccggcggcggaggatccggtggcggcgggtctggcggaggcggctcttccgaactgacccaggatcctgccgtgtccgtggccctgggacagacagtacggatcacctgtcagggcgactccctgcgctcttactacgcctcttggtaccagcagaagcctggccaggcccctgtgctggtgatctatggcaagaacaacagaccttctggcatccctgacagattctccggaagctccagcggcaacaccgcttctctcaccatcaccggcgctcaggccgaagatgaggccgactactactgccagtcctacgactcttcctcctaccactacgtgttcggcggcggcaccaagctgaccgtgctg

SEQ ID NO: 223 - protein, 579 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSAAAEVQLLESGGGLVQPGGSLRLSCAASGFTFSDYAMSWVRQAPGKGLEWVSAIRWHSSWTYYADSVKGRFTISRDNSKNTLYLQMNSLRAEDTAVYYCAKMKSTRHSIDDWGQGTLVTVSSGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCQSYDSSSYHYVFGGGTKLTVL

SEQ ID NO: 224 - DNA, 1737 bp, synthetic construct
  source 1..1737: mol_type = other DNA
gcttccaccaagggccctagcgtgtttcctctggcccctagctccaagagtacctccggcggcaccgcagctctgggctgcctcgtgaaggactacttccctgaacccgtgacagtgtcttggaactcgggcgctctgaccagtggagtgcacacctttccagccgtgctgcagtcttccggcctgtactccctgagctctgtggtgacagtgccttctagctctctgggcacacagacctacatctgcaacgtgaaccacaaaccctccaacacgaaagtggacaagaaggtggaacctaagtcctgcgacaagacccatacctgccctccttgccctgctcctgagctgctgggcgggccttccgtgtttctgttcccccctaaacccaaggacaccctaatgatctccagaacccctgaagttacatgcgtggtcgtggacgtgtcccacgaggatccagaggtgaagttcaactggtatgtggatggcgtggaagtgcacaacgccaagaccaagccaagagaagagcagtacggctccacctacagagtggtgtccgtgctcaccgtgctgcaccaggactggctgaacggcaaagagtacaagtgcaaggtgtccaacaaggccctgccagctcctatcgagaagaccatctctaaggctaagggccaacctagagagcctcaggtgtacaccctgcctccctctcgggacgagctgacaaagaaccaggtctccctgacctgcctggtcaaaggcttctacccctccgacatcgccgtcgagtgggagtccaatggccagcccgagaacaactacaagacaacacctcctgtgctggactccgacggctccttcttcctgtactccaagctgaccgtggataaatctcggtggcagcagggcaacgtgttctcctgctccgtgatgcacgaggctctgcataatcactacacccaaaagtctctgagcctgtctcctggaaagggcggaggcggatccgccgctgctgaagtgcagctgctggagtctggcggaggcctggtgcagcctgggggctcactgcggctgtcttgcgccgccagcggctttaccttcagcgattacgccatgtcctgggtgcggcaggccccaggcaagggcctggaatgggtgtctgccatcagatggcactccggctggacctactacgctgattctgtgaaaggccggttcaccatttcccgggataatagcaagaataccctgtacctgcagatgaactctctgcgggctgaggacaccgccgtgtactactgcgccaagatgaagtctaccagacacagcatcgacgactggggccaaggcaccctggtcaccgtgtcctccggcggcggaggatccggtggcggcgggtctggcggaggcggctcttccgaactgacccaggatcctgccgtgtccgtggccctgggacagacagtacggatcacctgtcagggcgactccctgcgctcttactacgcctcttggtaccagcagaagcctggccaggcccctgtgctggtgatctatggcaagaacaacagaccttctggcatccctgacagattctccggaagctccagcggcaacaccgcttctctcaccatcaccggcgctcaggccgaagatgaggccgactactactgccagtcctacgactcttcctcctaccactacgtgttcggcggcggcaccaagctgaccgtgctg

SEQ ID NO: 225 - protein, 579 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSAAAEVQLLESGGGLVQPGGSLRLSCAASGFTFSDYAMSWVRQAPGKGLEWVSAIRWHSGWTYYADSVKGRFTISRDNSKNTLYLQMNSLRAEDTAVYYCAKMKSTRHSIDDWGQGTLVTVSSGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCQSYDSSSYHYVFGGGTKLTVL

SEQ ID NO: 226 - DNA, 987 bp, synthetic construct
  source 1..987: mol_type = other DNA
gcctctaccaagggcccctctgtgttccctctggccccttcgtctaagtccacctctggcggcaccgctgctctgggctgcctggttaaggactacttcccagagccagtgacagtgagctggaactccggtgctctgacctccggcgtgcataccttccctgccgtgctgcagtcctctggcctgtactccctgtccagcgtggtgaccgtgcctagctcctccctgggcacacagacctacatctgcaacgtgaaccataagccttccaacaccaaagtggataagaaggtggaacccaagtcctgcgacaagacccacacctgcccaccttgtcctgcacctgaactgctggggggcccctccgtctttctgtttcctccaaaacctaaggataccctgatgatcagccggacacctgaagtgacctgcgtggtcgtcgacgtgtcccacgaagaccccgaggtgaagttcaactggtacgtggacggcgtggaagtgcacaacgccaagaccaagcccagagaagagcagtacggctctacctacagagtggtgtccgtgctgaccgttctgcaccaggactggctgaacggcaaagaatacaagtgcaaggtgtccaacaaggctctgcctgcccctatcgagaagaccatcagcaaggccaagggacagcctagagagcctcaagtgtacactctgcctccttctcgcgatgagctgacaaagaaccaggtgtccctgacctgtctcgtgaaaggcttctacccttctgacatcgccgtcgagtgggagtctaatggacagcccgagaacaactacaagaccacccctcccgtgctggactccgacggatctttcttcctgtattccaagctgacagtcgacaagtccagatggcagcagggcaacgtgttcagctgctccgtgatgcacgaggccctgcacaatcactacacccagaagagcttgtctctgtctcctggc

SEQ ID NO: 227 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 228 - DNA, 1068 bp, synthetic construct
  source 1..1068: mol_type = other DNA
cggaccgtggctgccccatctgtgttcatcttccctccttccgacgaacagctgaagtccggcaccgcctccgtggtgtgcctgctgaacaacttctaccccagagaggccaaggtgcagtggaaggtggacaatgctctgcagtccggcaactcccaagagtctgtcaccgagcaggacagtaaggactccacctactccctaagttctaccctcaccctgtccaaggccgactacgagaagcacaaggtgtacgcttgcgaggtgacccaccagggcctgtcttcccctgtgaccaagtccttcaaccggggcgagtgcggcggcggaggctctgccgctgctgaagtgcagctgctcgaatctggcggcggactggtgcagcctggcggctccctgagactgtcttgtgccgcttctggcttcaccttctccgattacgccatgtcctgggtgagacaggcccctggcaagggcctggaatgggtctccgccatcagatggcattcttcttggacctactacgccgattccgtgaagggcagattcaccatctccagagacaactcgaagaacaccctgtatctgcaaatgaactctctgagggccgaggacaccgctgtgtactactgtgctaagatgaaatctacaagacacagcatcgacgactggggccagggaacactggtgaccgtgtccagcggaggcggtggctctggaggaggcggcagcggcgggggcggatcttccgagctgacccaggatcctgccgtgtctgtggccctgggacagaccgtgcggatcacctgccagggcgactccctgagatcctactacgcctcatggtaccagcagaaaccaggccaggctcctgtgctggtgatctacggcaagaacaacagaccttctggcatccctgatagattttccggcagctcctctggtaataccgcttctctgacaatcaccggcgctcaggccgaggacgaggccgactactactgccagtcttacgactctagcagctaccactacgtgtttggcggcggcaccaagctgaccgtgctc

SEQ ID NO: 229 - protein, 356 aa, synthetic construct
RTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSAAAEVQLLESGGGLVQPGGSLRLSCAASGFTFSDYAMSWVRQAPGKGLEWVSAIRWHSSWTYYADSVKGRFTISRDNSKNTLYLQMNSLRAEDTAVYYCAKMKSTRHSIDDWGQGTLVTVSSGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCQSYDSSSYHYVFGGGTKLTVL

SEQ ID NO: 230 - DNA, 1068 bp, synthetic construct
  source 1..1068: mol_type = other DNA
cggaccgtggctgccccatctgtgttcatcttccctccttccgacgaacagctgaagtccggcaccgcctccgtggtgtgcctgctgaacaacttctaccccagagaggccaaggtgcagtggaaggtggacaatgctctgcagtccggcaactcccaagagtctgtcaccgagcaggacagtaaggactccacctactccctaagttctaccctcaccctgtccaaggccgactacgagaagcacaaggtgtacgcttgcgaggtgacccaccagggcctgtcttcccctgtgaccaagtccttcaaccggggcgagtgcggcggcggaggctctgccgctgctgaagtgcagctgctcgaatctggcggcggactggtgcagcctggcggctccctgagactgtcttgtgccgcttctggcttcaccttctccgattacgccatgtcctgggtgagacaggcccctggcaagggcctggaatgggtctccgccatcagatggcattctggctggacctactacgccgattccgtgaagggcagattcaccatctccagagacaactcgaagaacaccctgtatctgcaaatgaactctctgagggccgaggacaccgctgtgtactactgtgctaagatgaaatctacaagacacagcatcgacgactggggccagggaacactggtgaccgtgtccagcggaggcggtggctctggaggaggcggcagcggcgggggcggatcttccgagctgacccaggatcctgccgtgtctgtggccctgggacagaccgtgcggatcacctgccagggcgactccctgagatcctactacgcctcatggtaccagcagaaaccaggccaggctcctgtgctggtgatctacggcaagaacaacagaccttctggcatccctgatagattttccggcagctcctctggtaataccgcttctctgacaatcaccggcgctcaggccgaggacgaggccgactactactgccagtcttacgactctagcagctaccactacgtgtttggcggcggcaccaagctgaccgtgctc

SEQ ID NO: 231 - protein, 356 aa, synthetic construct
RTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSAAAEVQLLESGGGLVQPGGSLRLSCAASGFTFSDYAMSWVRQAPGKGLEWVSAIRWHSGWTYYADSVKGRFTISRDNSKNTLYLQMNSLRAEDTAVYYCAKMKSTRHSIDDWGQGTLVTVSSGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCQSYDSSSYHYVFGGGTKLTVL

SEQ ID NO: 232 - protein, 5 aa, synthetic construct
SFGMH

SEQ ID NO: 233 - protein, 17 aa, synthetic construct
YISSGSSTIYYGDTVKG

SEQ ID NO: 234 - protein, 15 aa, synthetic construct
EGGYYYGRSYYTMDY

SEQ ID NO: 235 - protein, 16 aa, synthetic construct
RSSQSIVHSNGNTYLE

SEQ ID NO: 236 - protein, 7 aa, synthetic construct
KVSNRFS

SEQ ID NO: 237 - protein, 9 aa, synthetic construct
FQGSHVPPT

SEQ ID NO: 238 - protein, 124 aa, synthetic construct
EVQLVESGGGLVQPGGSLRLSCSASGFTFSSFGMHWVRQTPGKGLEWVAYISSGSSTIYYGDTVKGRFTISRDNAKNSLFLQMSSLRAEDTAVYYCAREGGYYYGRSYYTMDYWGQGTTVTVSS

SEQ ID NO: 239 - protein, 112 aa, synthetic construct
DVVMTQSPLSLPATPGDPASISCRSSQSIVHSNGNTYLEWYLQKPGQSPKLLIYKVSNRFSGVPDRFSGSGSGTDFTLTISRVDAEDVGIYYCFQGSHVPPTFGPGTKLEIK

SEQ ID NO: 240 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSDLTVDKSRWQQGNVFSCSVMHEALHNHYTQESLSLSPG

SEQ ID NO: 241 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRKELTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 242 - protein, 1017 aa, synthetic construct
HHHHHHASEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTGAQPARLYWDDLKRKLSEKLDSTDFTGTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 243 - protein, 1037 aa, synthetic construct
HHHHHHASEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTGAQPALAGTESPVREEPGEDFPAARRLYWDDLKRKLSEKLDSTDFTGTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 244 - protein, 1037 aa, synthetic construct
HHHHHHASEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTGAQPALAGTESPAREEPEEDFPAAPRLYWDDLKRKLSEKLDTTDFTSTIKLLNENLYVPREAGSQKDENLALYIENQFREFKLSKVWRDQHFVKIQVKDSAQNSVIIVDKNGGLVYLVENPGGYVAYSKAATVTGKLVHANFGTKKDFEDLDSPVNGSIVIVRAGKITFAEKVANAESLNAIGVLIYMDQTKFPIVKADLSFFGHAHLGTGDPYTPGFPSFNHTQFPPSQSSGLPNIPVQTISRAAAEKLFGNMEGDCPSDWKTDSTCKMVTSENKSVKLTVSNVLKETKILNIFGVIKGFVEPDHYVVVGAQRDAWGPGAAKSSVGTALLLKLAQMFSDMVLKDGFQPSRSIIFASWSAGDFGSVGATEWLEGYLSSLHLKAFTYINLDKAVLGTSNFKVSASPLLYTLIEKTMQDVKHPVTGRSLYQDSNWASKVEKLTLDNAAFPFLAYSGIPAVSFCFCEDTDYPYLGTTMDTYKELVERIPELNKVARAAAEVAGQFVIKLTHDTELNLDYERYNSQLLLFLRDLNQYRADVKEMGLSLQWLYSARGDFFRATSRLTTDFRNAEKRDKFVMKKLNDRVMRVEYYFLSPYVSPKESPFRHVFWGSGSHTLSALLESLKLRRQNNSAFNETLFRNQLALATWTIQGAANALSGDVWDIDNEF

SEQ ID NO: 245 - DNA, 357 bp, synthetic construct
  source 1..357: mol_type = other DNA
gaagtgcaactactagaaagtggtggtggtctcgtgcagcccggaggctctctgcggctgtcttgtgctgcttctggcttcaccttctccgactacgccatgtcctgggtgcggcaagctcctggcaagtgcctggaatgggtctctgccatcagatggcactcctcttggacctactatgctgattccgtgaagggcagatttacaatctcccgggacaactccaagaacaccctgtacctgcagatgaacagcctgagagccgaggacaccgccgtgtactactgcgccaagatgaaaagcaccagacactggatcgacgattggggccagggcacactggtgaccgtgtccagc

SEQ ID NO: 246 - protein, 119 aa, synthetic construct
EVQLLESGGGLVQPGGSLRLSCAASGFTFSDYAMSWVRQAPGKCLEWVSAIRWHSSWTYYADSVKGRFTISRDNSKNTLYLQMNSLRAEDTAVYYCAKMKSTRHWIDDWGQGTLVTVSS

SEQ ID NO: 247 - DNA, 987 bp, synthetic construct
  source 1..987: mol_type = other DNA
gcctctaccaagggcccatccgtgtttccactggctccctccagcaagtccacaagcggaggaaccgccgctctgggatgtctggtgaaggactatttcccagagccagtgaccgtgtcctggaacagcggcgccctgacatccggagtgcatacctttccagctgtgctgcagtcttccggcctgtacagcctgagctctgtggtgacagtgccctccagctctctgggcacccagacatatatctgcaacgtgaatcacaagccatccaataccaaggtggacaagaaggtggagcccaagagctgtgataagacccatacatgccccccttgtcctgctccagagctgctgggaggaccaagcgtgttcctgtttccacccaagcctaaggacacactgatgatctctagaacacccgaggtgacctgcgtggtggtggacgtgtcccacgaggatcctgaggtgaagttcaactggtacgtggatggcgtggaggtgcataatgctaagaccaagcccagagaggagcagtacggcagcacatatcgcgtggtgtctgtgctgaccgtgctgcaccaggattggctgaacggcaaggagtataagtgcaaggtgagcaataaggccctgcccgctcctatcgagaagacaatctctaaggccaagggccagcctagagagccacaggtgtacaccctgcctccaagccgcgacgagctgacaaagaaccaggtgtctctgacctgtctggtgaagggcttctatccttctgatatcgctgtggagtgggagtccaatggccagccagagaacaattacaagaccacaccccctgtgctggactctgatggctccttctttctgtattccgacctgacagtggataagagccgctggcagcagggcaacgtgttttcttgttccgtgatgcatgaggctctgcacaatcattacacccaggagagcctgtctctgtcccctggc

SEQ ID NO: 248 - protein, 329 aa, synthetic construct; identical to SEQ ID NO: 240

SEQ ID NO: 249 - DNA, 1737 bp, synthetic construct
  source 1..1737: mol_type = other DNA
gccagcaccaaaggcccctcagtctttcctctggccccttcttccaagtccacctccggcggcaccgccgcactgggctgtctggtcaaggactacttccctgagcccgtgacagtgtcctggaactctggtgccctgacctctggcgtgcacacatttcctgctgtgctacagtcttccggcctgtactccctctcgtctgtggtgaccgtcccttcttctagtctgggaacccaaacctacatctgcaacgtgaaccacaagccctctaacaccaaagtggacaagaaggtggaacccaagtcctgcgacaagacccatacctgccctccttgtcctgcccccgagctgctgggcggcccctccgtgtttctgttcccccctaagcctaaggacaccctgatgatctctcggacccctgaggtgacctgcgtggtggtggacgtgtctcatgaggaccctgaagtgaagttcaactggtatgtggatggcgtggaagtgcacaacgccaaaaccaagcctagagaagagcagtacggctccacctacagagtggtctccgtgctgaccgtgctgcaccaggattggctgaacggaaaagagtacaagtgcaaggtgtccaacaaggctctgcctgctccaatcgagaagaccatctccaaggccaagggccagcctagagagcctcaggtgtacacactccctccatctagaaaggaactgaccaaaaaccaggtgtccctgacatgcctagtgaagggcttctacccttccgacatcgccgtcgagtgggagtccaacggccagcctgagaacaactacaagaccacccctcctgtgctgaagtctgacggctccttcttcctgtactccaagctgacagttgataagtctcggtggcagcagggcaatgtgttcagctgctccgtgatgcacgaggctctgcacaaccactacacccagaagagtctctccctgtcaccaggcaaaggcggaggtggctctgctgctgccgaagtgcagctgctggaatctggcggcggtctggtgcagcctggcggctccctgcggctgtcttgcgctgcctccggattcaccttcagcgactacgccatgtcctgggtgagacaggctcccggcaaaggcctggaatgggtgagcgccatcagatggcactcctcctggacctactacgccgactccgtgaaaggacggttcacaattagcagagacaacagcaagaacaccctgtacctgcagatgaactctctgagagccgaggataccgctgtgtactactgcgccaagatgaagagcacccggcactggatcgacgactggggacagggcaccctggtgaccgtgtcttccggaggcggtggatctggtggtggcggatcaggtggaggcgggagctccgagctgacccaagatcctgctgtgtctgtggccctgggccaaacagtgcggatcacctgccagggcgactctctgagatcctattatgcttcctggtaccagcagaaacccggccaggccccagtgctggtgatctacggcaagaacaatcggcctagcggcatccctgacaggttcagcggctcatcctccggcaacaccgcctctctgacaatcaccggcgcccaggccgaggacgaggccgattactactgccagtcctacgactcctcttcttaccactacgtgttcggcggcggaaccaagctgacagtgctg

SEQ ID NO: 250 - protein, 579 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRKELTKNQVSLTCLVKGFYPSDIAVEWESNGQPENNYKTTPPVLKSDGSFFLYSKLTVDKSRWQQGNVFSCSVMHEALHNHYTQKSLSLSPGKGGGGSAAAEVQLLESGGGLVQPGGSLRLSCAASGFTFSDYAMSWVRQAPGKGLEWVSAIRWHSSWTYYADSVKGRFTISRDNSKNTLYLQMNSLRAEDTAVYYCAKMKSTRHWIDDWGQGTLVTVSSGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCQSYDSSSYHYVFGGGTKLTVL

SEQ ID NO: 251 - DNA, 1737 bp, synthetic construct
  source 1..1737: mol_type = other DNA
gcctccaccaagggaccatccgtgttcccactggccccatccagcaagagcacatctggaggaaccgccgctctgggatgtctggtgaaggactacttcccagagcccgtgaccgtgagctggaactctggcgctctgacatccggcgtgcatacctttcccgccgtgctgcagtcttccggcctgtacagcctgagctctgtggtgacagtgccttccagctctctgggcacccagacatatatctgcaacgtgaatcacaagccctccaataccaaggtggacaagaaggtggagcctaagagctgtgataagacccatacatgccccccttgtcctgctccagagctgctgggaggaccttccgtgttcctgtttccacccaagccaaaggacacactgatgatctctcggacacctgaggtgacctgcgtggtggtggacgtgtcccacgaggatccagaggtgaagttcaactggtacgtggatggcgtggaggtgcataatgccaagaccaagcctagagaggagcagtacggcagcacatatcgcgtggtgtctgtgctgaccgtgctgcaccaggactggctgaacggcaaggagtataagtgcaaggtgagcaataaggctctgcccgcccctatcgagaagacaatctctaaggctaagggccagcctagggagccacaggtgtacaccctgcctccatctcggaaggagctgacaaagaaccaggtgtccctgacctgtctggtgaagggcttctatccatccgatatcgccgtggagtgggagagcaatggccagcccgagaacaattacaagaccacaccccctgtgctgaagtccgacggcagcttctttctgtattctaagctgacagtggataagtccaggtggcagcagggcaacgtgttttcctgcagcgtgatgcatgaggctctgcacaatcattacacccagaagtctctgtccctgagcccaggcaagggaggaggaggaagcgccgctgccgaagtccagctgctggagtctggcggcggactggtgcagccaggaggctctctgaggctgtcctgtgctgccagcggcttcacattttccgactacgctatgagctgggtgagacaggcccctggcaagggcctggaatgggtgtccgctatccgctggcacagcggctggacctactatgccgacagcgtgaagggcaggttcaccatctctcgcgataactccaagaataccctgtatctgcagatgaactctctgagggccgaagacaccgccgtgtactattgcgccaagatgaagtccacccggcattggatcgacgattggggtcagggcacactggtcaccgtgtccagcggaggaggaggatctggcggaggaggctccggcggcggcggctcttccgagctgacccaggaccctgccgtgtccgtggccctgggacagacagtgaggatcacctgtcagggcgattctctgcggtcctactatgcttcctggtaccagcagaagccaggacaggctcccgtgctggtcatctacggcaagaacaataggccaagcggcatccccgaccggttctctggcagctcttccggcaataccgcctccctgaccatcacaggagctcaggctgaggacgaggctgattactattgccagtcttacgatagctcttcctaccactacgtgttcggcggcggcacaaagctgaccgtgctg

SEQ ID NO: 252 - protein, 579 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRKELTKNQVSLTCLVKGFYPSDIAVEWESNGQPENNYKTTPPVLKSDGSFFLYSKLTVDKSRWQQGNVFSCSVMHEALHNHYTQKSLSLSPGKGGGGSAAAEVQLLESGGGLVQPGGSLRLSCAASGFTFSDYAMSWVRQAPGKGLEWVSAIRWHSGWTYYADSVKGRFTISRDNSKNTLYLQMNSLRAEDTAVYYCAKMKSTRHWIDDWGQGTLVTVSSGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCQSYDSSSYHYVFGGGTKLTVL

SEQ ID NO: 253 - protein, 763 aa, synthetic construct
MMDQARSAFSNLFGGEPLSYTRFSLARQVDGDNSHVEMKLAADEEENADNNMKASVRKPKRFNGRLCFAAIALVIFFLIGFMSGYLGYCKRVEQKEECVKLAETEETDKSETMETEDVPTSSRLYWADLKTLLSEKLNSIEFADTIKQLSQNTYTPREAGSQKDESLAYYIENQFHEFKFSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGYEEPDRYVVVGAQRDALGAGVAAKSSVGTGLLLKLAQVFSDMISKDGFRPSRSIIFASWTAGDFGAVGATEWLEGYLSSLHLKAFTYINLDKVVLGTSNFKVSASPLLYTLMGKIMQDVKHPVDGKSLYRDSNWISKVEKLSFDNAAYPFLAYSGIPAVSFCFCEDADYPYLGTRLDTYEALTQKVPQLNQMVRTAAEVAGQLIIKLTHDVELNLDYEMYNSKLLSFMKDLNQFKTDIRDMGLSLQWLYSARGDYFRATSRLTTDFHNAEKTNRFVMREINDRIMKVEYHFLSPYVSPRESPFRHIFWGSGSHTLSALVENLKLRQKNITAFNETLFRNQLALATWTIQGVANALSGDIWNIDNEF

SEQ ID NO: 254 - protein, 17 aa, synthetic construct
  UNSURE 3: note = S or R
  UNSURE 4: note = G or W
  UNSURE 5: note = S or H
  UNSURE 6: note = G or S
  UNSURE 7: note = G or S
  UNSURE 8: note = S or W
AIXXXXXXTYYADSVKG

SEQ ID NO: 255 - protein, 18 aa, synthetic construct
GGGGSGGGGSGGGGSAAA

SEQ ID NO: 256 - protein, 760 aa, Homo sapiens
MMDQARSAFSNLFGGEPLSYTRFSLARQVDGDNSHVEMKLAVDEEENADNNTKANVTKPKRCSGSICYGTIAVIVFFLIGFMIGYLGYCKGVEPKTECERLAGTESPVREEPGEDFPAARRLYWDDLKRKLSEKLDSTDFTGTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 257 - protein, 760 aa, Macaca fascicularis
MMDQARSAFSNLFGGEPLSYTRFSLARQVDGDNSHVEMKLGVDEEENTDNNTKPNGTKPKRCGGNICYGTIAVIIFFLIGFMIGYLGYCKGVEPKTECERLAGTESPAREEPEEDFPAAPRLYWDDLKRKLSEKLDTTDFTSTIKLLNENLYVPREAGSQKDENLALYIENQFREFKLSKVWRDQHFVKIQVKDSAQNSVIIVDKNGGLVYLVENPGGYVAYSKAATVTGKLVHANFGTKKDFEDLDSPVNGSIVIVRAGKITFAEKVANAESLNAIGVLIYMDQTKFPIVKADLSFFGHAHLGTGDPYTPGFPSFNHTQFPPSQSSGLPNIPVQTISRAAAEKLFGNMEGDCPSDWKTDSTCKMVTSENKSVKLTVSNVLKETKILNIFGVIKGFVEPDHYVVVGAQRDAWGPGAAKSSVGTALLLKLAQMFSDMVLKDGFQPSRSIIFASWSAGDFGSVGATEWLEGYLSSLHLKAFTYINLDKAVLGTSNFKVSASPLLYTLIEKTMQDVKHPVTGRSLYQDSNWASKVEKLTLDNAAFPFLAYSGIPAVSFCFCEDTDYPYLGTTMDTYKELVERIPELNKVARAAAEVAGQFVIKLTHDTELNLDYERYNSQLLLFLRDLNQYRADVKEMGLSLQWLYSARGDFFRATSRLTTDFRNAEKRDKFVMKKLNDRVMRVEYYFLSPYVSPKESPFRHVFWGSGSHTLSALLESLKLRRQNNSAFNETLFRNQLALATWTIQGAANALSGDVWDIDNEF

SEQ ID NO: 258 - protein, 329 aa, synthetic construct; identical to SEQ ID NO: 165

SEQ ID NO: 259 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLTCLVEGFYPSDIAVEWESNGQPENNYKTTPPVLDSDGSFFLYSDLTVDKSRWQQGNVFSCSVMHEALHNHYTQESLSLSPGK

SEQ ID NO: 260 - protein, 329 aa, synthetic construct; identical to SEQ ID NO: 173

SEQ ID NO: 261 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRKKLTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 262 - DNA, 723 bp, synthetic construct
  source 1..723: mol_type = other DNA
gaggtgcagctgctggaatccggcggcggactggtgcagccaggaggctctctgcggctgtcctgtgctgccagcggcttcacattttccgactatgctatgagctgggtgagacaggctcctggcaagtgcctggagtgggtgtccgctatccgctggcacagctcttggacctattacgccgacagcgtgaagggcagattcacaatctctcgcgataactccaagaataccctgtacctgcagatgaacagcctgagggctgaggatacagccgtgtattactgcgccaagatgaagtctacccggcattggatcgacgattggggccagggcaccctggtgacagtgtcctctggaggaggaggatctggaggaggaggctccggcggcggcggctcttccgagctgacccaggaccctgccgtgtccgtggccctgggccagaccgtgagaatcacatgtcagggcgattccctgcgcagctattacgcttcctggtatcagcagaagccaggacaggctcccgtgctggtcatctacggcaagaacaataggccaagcggcatccccgaccggttctctggcagctcttccggcaacaccgcttccctgaccatcacaggagctcaggctgaggacgaggctgattattactgccagagctacgatagctcttcctatcattacgtgtttggctgcggcaccaagctgacagtgctg

SEQ ID NO: 263 - protein, 241 aa, synthetic construct
EVQLLESGGGLVQPGGSLRLSCAASGFTFSDYAMSWVRQAPGKCLEWVSAIRWHSSWTYYADSVKGRFTISRDNSKNTLYLQMNSLRAEDTAVYYCAKMKSTRHWIDDWGQGTLVTVSSGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCQSYDSSSYHYVFGCGTKLTVL

SEQ ID NO: 264 - DNA, 324 bp, synthetic construct
  source 1..324: mol_type = other DNA
tcttccgagctgacccaggaccctgccgtgtccgtggccctgggccagaccgtgagaatcacatgtcagggcgattccctgcgcagctattacgcttcctggtatcagcagaagccaggacaggctcccgtgctggtcatctacggcaagaacaataggccaagcggcatccccgaccggttctctggcagctcttccggcaacaccgcttccctgaccatcacaggagctcaggctgaggacgaggctgattattactgccagagctacgatagctcttcctatcattacgtgtttggctgcggcaccaagctgacagtgctg

SEQ ID NO: 265 - protein, 108 aa, synthetic construct; identical to SEQ ID NO: 147

SEQ ID NO: 266 - DNA, 357 bp, synthetic construct
  source 1..357: mol_type = other DNA
gaggtgcagctgctggaatccggcggcggactggtgcagccaggaggctctctgcggctgtcctgtgctgccagcggcttcacattttccgactatgctatgagctgggtgagacaggctcctggcaagtgcctggagtgggtgtccgctatccgctggcacagctcttggacctattacgccgacagcgtgaagggcagattcacaatctctcgcgataactccaagaataccctgtacctgcagatgaacagcctgagggctgaggatacagccgtgtattactgcgccaagatgaagtctacccggcattggatcgacgattggggccagggcaccctggtgacagtgtcctct

SEQ ID NO: 267 - protein, 119 aa, synthetic construct; identical to SEQ ID NO: 246

SEQ ID NO: 268 - DNA, 2109 bp, synthetic construct
  source 1..2109: mol_type = other DNA
gaggtgcagctggtggagtctggaggaggactggtgcagcctggaggctctctgagactgagctgctctgcttccggcttcacattttccagctttggcatgcattgggtgcgccagaccccaggcaagggactggagtgggtggcctacatctcttccggcagctctaccatctactatggcgacacagtgaagggcaggttcaccatctcccgggataacgctaagaatagcctgtttctgcagatgtccagcctgagagctgaggacacagccgtgtactattgcgccagagagggcggctactattacggccgctcctattacacaatggactattggggccagggcaccacagtgacagtgtcttccgcctccaccaagggaccatccgtgttcccactggccccaagctctaagagcacctctggaggaacagccgctctgggatgtctggtgaaggactacttcccagagcccgtgaccgtgtcttggaactccggcgctctgacctctggcgtgcacacatttcccgccgtgctgcagtccagcggcctgtattccctgtcttccgtggtgacagtgcctagctcttccctgggcacccagacatacatctgcaacgtgaatcacaagccctccaataccaaggtggacaagaaggtggagcctaagagctgtgataagacccatacatgccccccttgtcctgctccagagctgctgggaggacctagcgtgttcctgtttccacccaagccaaaggacaccctgatgatctctcgcacccctgaggtgacatgcgtggtggtggacgtgtcccacgaggatccagaggtgaagtttaactggtacgtggatggcgtggaggtgcataatgccaagaccaagcctagggaggagcagtatggcagcacataccgggtggtgtctgtgctgaccgtgctgcaccaggactggctgaacggcaaggagtacaagtgcaaggtgagcaataaggctctgcccgcccctatcgagaagacaatctctaaggctaagggccagcctagggagccacaggtgtataccctgcctccatctcggaaggagctgacaaagaaccaggtgtccctgacctgtctggtgaagggcttctacccatccgatatcgccgtggagtgggagagcaatggccagcccgagaacaattataagaccacaccccctgtgctgaagagcgacggctctttctttctgtactctaagctgacagtggataagtccaggtggcagcagggcaacgtgttttcctgcagcgtgatgcatgaggctctgcacaatcattacacccagaagtctctgtccctgagcccaggcaagggaggaggaggaagcgccgctgccgaggtgcagctgctggaatccggcggcggactggtgcagccaggaggctctctgcggctgtcctgtgctgccagcggcttcacattttccgactatgctatgagctgggtgagacaggctcctggcaagtgcctggagtgggtgtccgctatccgctggcacagctcttggacctattacgccgacagcgtgaagggcagattcacaatctctcgcgataactccaagaataccctgtacctgcagatgaacagcctgagggctgaggatacagccgtgtattactgcgccaagatgaagtctacccggcattggatcgacgattggggccagggcaccctggtgacagtgtcctctggaggaggaggatctggaggaggaggctccggcggcggcggctcttccgagctgacccaggaccctgccgtgtccgtggccctgggccagaccgtgagaatcacatgtcagggcgattccctgcgcagctattacgcttcctggtatcagcagaagccaggacaggctcccgtgctggtcatctacggcaagaacaataggccaagcggcatccccgaccggttctctggcagctcttccggcaacaccgcttccctgaccatcacaggagctcaggctgaggacgaggctgattattactgccagagctacgatagctcttcctatcattacgtgtttggctgcggcaccaagctgacagtgctg

SEQ ID NO: 269 - protein, 703 aa, synthetic construct
EVQLVESGGGLVQPGGSLRLSCSASGFTFSSFGMHWVRQTPGKGLEWVAYISSGSSTIYYGDTVKGRFTISRDNAKNSLFLQMSSLRAEDTAVYYCAREGGYYYGRSYYTMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRKELTKNQVSLTCLVKGFYPSDIAVEWESNGQPENNYKTTPPVLKSDGSFFLYSKLTVDKSRWQQGNVFSCSVMHEALHNHYTQKSLSLSPGKGGGGSAAAEVQLLESGGGLVQPGGSLRLSCAASGFTFSDYAMSWVRQAPGKCLEWVSAIRWHSSWTYYADSVKGRFTISRDNSKNTLYLQMNSLRAEDTAVYYCAKMKSTRHWIDDWGQGTLVTVSSGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCQSYDSSSYHYVFGCGTKLTVL

SEQ ID NO: 270 - DNA, 723 bp, synthetic construct
  source 1..723: mol_type = other DNA
gaggtgcagctgctggaatccggcggcggactggtgcagccaggaggctccctgcggctgagctgtgctgcctctggcttcaccttctccgactatgctatgtcttgggtgagacaggctcctggcaagtgcctggagtgggtgtccgccatcaggtggcactctggctggacctattacgccgacagcgtgaagggcagattcacaatctcccgcgataacagcaagaataccctgtacctgcagatgaactctctgagggctgaggatacagccgtgtattactgcgccaagatgaagtccacccggcattggatcgacgattggggccagggcaccctggtgacagtgagctctggaggaggaggatccggaggaggaggaagcggcggcggcggctccagcgagctgacccaggaccctgccgtgtccgtggccctgggccagaccgtgagaatcacatgtcagggcgatagcctgcgctcttattacgcctcctggtatcagcagaagccaggacaggctcccgtgctggtcatctacggcaagaacaataggccaagcggcatccccgaccggttctctggctcttccagcggcaacaccgcctccctgaccatcacaggagctcaggctgaggacgaggctgattattactgccagtcttacgattcttccagctatcattacgtgtttggctgcggcaccaagctgacagtgctg

SEQ ID NO: 271 - protein, 241 aa, synthetic construct
EVQLLESGGGLVQPGGSLRLSCAASGFTFSDYAMSWVRQAPGKCLEWVSAIRWHSGWTYYADSVKGRFTISRDNSKNTLYLQMNSLRAEDTAVYYCAKMKSTRHWIDDWGQGTLVTVSSGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCQSYDSSSYHYVFGCGTKLTVL

SEQ ID NO: 272 - DNA, 324 bp, synthetic construct
  source 1..324: mol_type = other DNA
tccagcgagctgacccaggaccctgccgtgtccgtggccctgggccagaccgtgagaatcacatgtcagggcgatagcctgcgctcttattacgcctcctggtatcagcagaagccaggacaggctcccgtgctggtcatctacggcaagaacaataggccaagcggcatccccgaccggttctctggctcttccagcggcaacaccgcctccctgaccatcacaggagctcaggctgaggacgaggctgattattactgccagtcttacgattcttccagctatcattacgtgtttggctgcggcaccaagctgacagtgctg

SEQ ID NO: 273 - protein, 108 aa, synthetic construct; identical to SEQ ID NO: 147

SEQ ID NO: 274 - DNA, 357 bp, synthetic construct
  source 1..357: mol_type = other DNA
gaggtgcagctgctggaatccggcggcggactggtgcagccaggaggctccctgcggctgagctgtgctgcctctggcttcaccttctccgactatgctatgtcttgggtgagacaggctcctggcaagtgcctggagtgggtgtccgccatcaggtggcactctggctggacctattacgccgacagcgtgaagggcagattcacaatctcccgcgataacagcaagaataccctgtacctgcagatgaactctctgagggctgaggatacagccgtgtattactgcgccaagatgaagtccacccggcattggatcgacgattggggccagggcaccctggtgacagtgagctct

SEQ ID NO: 275 - protein, 119 aa, synthetic construct
EVQLLESGGGLVQPGGSLRLSCAASGFTFSDYAMSWVRQAPGKCLEWVSAIRWHSGWTYYADSVKGRFTISRDNSKNTLYLQMNSLRAEDTAVYYCAKMKSTRHWIDDWGQGTLVTVSS

SEQ ID NO: 276 - DNA, 2109 bp, synthetic construct
  source 1..2109: mol_type = other DNA
gaggtgcagctggtggagtctggaggaggactggtgcagcctggaggctccctgagactgtcttgctccgcctccggcttcacattttccagctttggcatgcattgggtgcgccagaccccaggcaagggactggagtgggtggcctacatctcttccggcagctctaccatctactatggcgacacagtgaagggcaggttcaccatctcccgggataacgctaagaatagcctgtttctgcagatgtccagcctgagagctgaggacacagccgtgtactattgcgccagagagggcggctactattacggccgcagctattacacaatggactattggggccagggcaccacagtgacagtgtcttccgcttctaccaagggaccatccgtgttcccactggccccaagctctaagtctacctccggaggaacagccgctctgggatgtctggtgaaggactacttcccagagccagtgaccgtgtcctggaacagcggcgctctgacctccggagtgcacacatttcccgccgtgctgcagtccagcggcctgtatagcctgtcttccgtggtgacagtgcctagctcttccctgggcacccagacatacatctgcaacgtgaatcacaagccctccaataccaaggtggacaagaaggtggagcctaagagctgtgataagacccatacatgccccccttgtcctgctccagagctgctgggaggaccttccgtgttcctgtttccacccaagccaaaggacaccctgatgatctctcgcacccctgaggtgacatgcgtggtggtggacgtgtcccacgaggatccagaggtgaagtttaactggtacgtggatggcgtggaggtgcataatgccaagaccaagcctagggaggagcagtatggctctacataccgggtggtgtccgtgctgaccgtgctgcaccaggactggctgaacggcaaggagtacaagtgcaaggtgagcaataaggctctgcccgcccctatcgagaagacaatctctaaggctaagggccagcctagggagccacaggtgtataccctgcctccatctcggaaggagctgacaaagaaccaggtgtccctgacctgtctggtgaagggcttctacccatccgatatcgccgtggagtgggagagcaatggccagcccgagaacaattataagaccacaccccctgtgctgaagtctgacggctccttctttctgtactctaagctgacagtggataagtccaggtggcagcagggcaacgtgtttagctgctctgtgatgcatgaggctctgcacaatcattacacccagaagtccctgagcctgtctccaggcaagggaggaggaggaagcgccgctgccgaggtgcagctgctggaatccggcggcggactggtgcagccaggaggctccctgcggctgagctgtgctgcctctggcttcaccttctccgactatgctatgtcttgggtgagacaggctcctggcaagtgcctggagtgggtgtccgccatcaggtggcactctggctggacctattacgccgacagcgtgaagggcagattcacaatctcccgcgataacagcaagaataccctgtacctgcagatgaactctctgagggctgaggatacagccgtgtattactgcgccaagatgaagtccacccggcattggatcgacgattggggccagggcaccctggtgacagtgagctctggaggaggaggatccggaggaggaggaagcggcggcggcggctccagcgagctgacccaggaccctgccgtgtccgtggccctgggccagaccgtgagaatcacatgtcagggcgatagcctgcgctcttattacgcctcctggtatcagcagaagccaggacaggctcccgtgctggtcatctacggcaagaacaataggccaagcggcatccccgaccggttctctggctcttccagcggcaacaccgcctccctgaccatcacaggagctcaggctgaggacgaggctgattattactgccagtcttacgattcttccagctatcattacgtgtttggctgcggcaccaagctgacagtgctg

SEQ ID NO: 277 - protein, 703 aa, synthetic construct
EVQLVESGGGLVQPGGSLRLSCSASGFTFSSFGMHWVRQTPGKGLEWVAYISSGSSTIYYGDTVKGRFTISRDNAKNSLFLQMSSLRAEDTAVYYCAREGGYYYGRSYYTMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRKELTKNQVSLTCLVKGFYPSDIAVEWESNGQPENNYKTTPPVLKSDGSFFLYSKLTVDKSRWQQGNVFSCSVMHEALHNHYTQKSLSLSPGKGGGGSAAAEVQLLESGGGLVQPGGSLRLSCAASGFTFSDYAMSWVRQAPGKCLEWVSAIRWHSGWTYYADSVKGRFTISRDNSKNTLYLQMNSLRAEDTAVYYCAKMKSTRHWIDDWGQGTLVTVSSGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCQSYDSSSYHYVFGCGTKLTVL

SEQ ID NO: 278 - DNA, 372 bp, synthetic construct
  source 1..372: mol_type = other DNA
gaggtgcagctggtggagtccggaggaggactggtgcagcctggaggctccctgaggctgagctgctctgcctccggcttcacattttccagctttggcatgcattgggtgcggcagaccccaggcaagggactggagtgggtggcttatatctcttccggcagctctaccatctactatggcgacacagtgaagggcaggttcaccatcagccgggataacgccaagaactccctgtttctgcagatgtccagcctgagggccgaggacacagccgtgtactattgcgctagagagggcggctactattacggccgcagctattacacaatggactattggggccagggcaccacagtgacagtgtcttcc

SEQ ID NO: 279 - DNA, 336 bp, synthetic construct
  source 1..336: mol_type = other DNA
gatgtggtgatgactcaaagtccactaagtctgcctgctacccctggcgatcctgcctctatctcctgccggtcctctcagtccatcgtgcactctaacggtaatacatatctggaatggtacctgcagaaaccaggccagtctcctaagctgctgatctacaaggtgtccaaccggttttctggcgtgcccgatagattttccggcagcggctctggaaccgacttcaccctcaccatctctagagtggatgccgaggacgtgggaatctactactgcttccagggcagtcatgtgcctcctaccttcggccctggcacaaagctggagatcaag

SEQ ID NO: 280 - DNA, 987 bp, synthetic construct
  source 1..987: mol_type = other DNA
gcctccaccaagggcccaagcgtgttcccactggctcccagctctaagagcacctctggaggaacagccgctctgggatgtctggtgaaggactacttcccagagcccgtgaccgtgtcttggaactccggcgccctgacctccggagtgcacacatttccagctgtgctgcagtccagcggcctgtatagcctgtcttccgtggtgacagtgcccagctcttccctgggcacccagacatacatctgcaacgtgaatcacaagccatccaataccaaggtggacaagaaggtggagcccaagagctgtgataagacccatacatgccccccttgtcctgctccagagctgctgggaggaccatccgtgttcctgtttccacccaagcctaaggacacactgatgatctctagaacccccgaggtgacatgcgtggtggtggacgtgtcccacgaggatcctgaggtgaagtttaactggtacgtggatggcgtggaggtgcataatgctaagaccaagcccagggaggagcagtatggctctacataccgggtggtgtccgtgctgaccgtgctgcaccaggattggctgaacggcaaggagtacaagtgcaaggtgagcaataaggccctgcccgctcctatcgagaagacaatctctaaggccaagggccagcctagagagccacaggtgtataccctgcctccaagccgcgacgagctgacaaagaaccaggtgtctctgacctgtctggtgaagggcttctacccttctgatatcgctgtggagtgggagtccaatggccagccagagaacaattataagaccacaccccctgtgctggacagcgatggctctttctttctgtactccgacctgacagtggataagagccgctggcagcagggcaacgtgttttcctgtagcgtgatgcatgaggccctgcacaatcattacacccaggagtctctgtccctgagccctggc

SEQ ID NO: 281 - DNA, 990 bp, synthetic construct
  source 1..990: mol_type = other DNA
gcctccaccaagggaccatccgtgttcccactggccccaagctctaagagcacctctggaggaacagccgctctgggatgtctggtgaaggactacttcccagagcccgtgaccgtgtcttggaactccggcgctctgacctctggcgtgcacacatttcccgccgtgctgcagtccagcggcctgtattccctgtcttccgtggtgacagtgcctagctcttccctgggcacccagacatacatctgcaacgtgaatcacaagccctccaataccaaggtggacaagaaggtggagcctaagagctgtgataagacccatacatgccccccttgtcctgctccagagctgctgggaggacctagcgtgttcctgtttccacccaagccaaaggacaccctgatgatctctcgcacccctgaggtgacatgcgtggtggtggacgtgtcccacgaggatccagaggtgaagtttaactggtacgtggatggcgtggaggtgcataatgccaagaccaagcctagggaggagcagtatggcagcacataccgggtggtgtctgtgctgaccgtgctgcaccaggactggctgaacggcaaggagtacaagtgcaaggtgagcaataaggctctgcccgcccctatcgagaagacaatctctaaggctaagggccagcctagggagccacaggtgtataccctgcctccatctcggaaggagctgacaaagaaccaggtgtccctgacctgtctggtgaagggcttctacccatccgatatcgccgtggagtgggagagcaatggccagcccgagaacaattataagaccacaccccctgtgctgaagagcgacggctctttctttctgtactctaagctgacagtggataagtccaggtggcagcagggcaacgtgttttcctgcagcgtgatgcatgaggctctgcacaatcattacacccagaagtctctgtccctgagcccaggcaag

SEQ ID NO: 282 - DNA, 321 bp, synthetic construct
  source 1..321: mol_type = other DNA
cggaccgtggccgctccatccgtcttcatcttccccccctccgacgaacagctgaagtccggcaccgcttctgtggtgtgcctgctgaacaacttctaccctagagaagccaaagtgcagtggaaggtggacaacgctctgcagagcggcaactcccaagagagcgtcaccgagcaggactccaaggactctacctactccctgtccagcaccctgacactgtctaaggccgactacgagaagcacaaggtgtacgcctgcgaggtgacccaccagggcctgagctcccctgtgaccaagtccttcaacagaggcgagtgt

SEQ ID NO: 283 - protein, 330 aa, synthetic construct; identical to SEQ ID NO: 95

SEQ ID NO: 284 - protein, 107 aa, synthetic construct [sequence omitted]

SEQ ID NO: 285 - protein, 124 aa, synthetic construct
EVQLVESGGGLVQPGGSLRLSCSASGFTFSSFGMHWVRQAPGKGLEWVAYISSGSSTIYYGDTVKGRFTISRDNAKNSLFLQMSSLRAEDTAVYYCAREGGYYYGRSYYTMDYWGQGTTVTVSS

SEQ ID NO: 286 - protein, 112 aa, synthetic construct
DVVMTQSPLSLPVTPGAPASISCRSSQSIVHSNGNTYLEWYLQKPGQSPKLLIYKVSNRFSGVPDRFSGSGSGTDFTLRISRVEAEDVGIYYCFQGSHVPPTFGPGTKLEIK

SEQ ID NO: 287 - protein, 14 aa, synthetic construct
GGGGSGGGGSGGGG

SEQ ID NO: 288 - protein, 24 aa, synthetic construct
GGGGSGGGGSGGGGSGGGGSGGGG

SEQ ID NO: 289 - protein, 251 aa, synthetic construct
EVQLLESGGGLVQPGGSLRLSCAASGFTFSDYAMSWVRQAPGKGLEWVSAIRWHSSWTYYADSVKGRFTISRDNSKNTLYLQMNSLRAEDTAVYYCAKMKSTRHWIDDWGQGTLVTVSSGGGGSGGGGSGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCQSYDSSSYHYVFGGGTKLTVL

SEQ ID NO: 290 - protein, 251 aa, synthetic construct
EVQLLESGGGLVQPGGSLRLSCAASGFTFSDYAMSWVRQAPGKGLEWVSAIRWHSGWTYYADSVKGRFTISRDNSKNTLYLQMNSLRAEDTAVYYCAKMKSTRHWIDDWGQGTLVTVSSGGGGSGGGGSGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCQSYDSSSYHYVFGGGTKLTVL

SEQ ID NO: 291 - protein, 251 aa, synthetic construct
EVQLLESGGGLVQPGGSLRLSCAASGFTFSDYAMSWVRQAPGKCLEWVSAIRWHSSWTYYADSVKGRFTISRDNSKNTLYLQMNSLRAEDTAVYYCAKMKSTRHWIDDWGQGTLVTVSSGGGGSGGGGSGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCQSYDSSSYHYVFGCGTKLTVL

SEQ ID NO: 292 - protein, 251 aa, synthetic construct
EVQLLESGGGLVQPGGSLRLSCAASGFTFSDYAMSWVRQAPGKCLEWVSAIRWHSGWTYYADSVKGRFTISRDNSKNTLYLQMNSLRAEDTAVYYCAKMKSTRHWIDDWGQGTLVTVSSGGGGSGGGGSGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCQSYDSSSYHYVFGCGTKLTVL

SEQ ID NO: 293 - DNA, 1359 bp, synthetic construct
  source 1..1359: mol_type = other DNA
gaggtgcagctggttgaatctggcggcggcttggttcagcctggcggctctctgaggttgtcctgctctgcctctggcttcaccttctcctccttcggcatgcactgggtgcggcagacacctggcaagggcctggaatgggtcgcctacatctcctccggctcctccaccatctactatggcgacaccgtgaagggcagattcaccatctcccgggacaacgccaagaacagcctgttcctgcagatgtccagcctgagagccgaggacaccgccgtgtactactgcgccagagagggcggctactactatggcaggtcctactacaccatggactactggggccagggcaccaccgtgaccgtgtcctctgcctctaccaagggcccctctgtgtttcctctggctccttccagcaagtctacctctggcggcacagctgctctgggctgcctggtcaaggactactttcctgagcctgtgaccgtgtcttggaactctggcgctctgacatctggcgtgcacacctttccagctgtgctgcagtcctccggcctgtactctctgtcctctgtcgtgaccgtgccttccagctctctgggcacccagacctacatctgcaacgtgaaccacaagccttccaacaccaaggtggacaagaaggtggaacccaagtcctgcgacaagacccacacctgtcctccatgtcctgctccagaactgctcggcggcccttccgtgttcctgtttcctccaaagcctaaggacaccctgatgatctctcggacccctgaagtgacctgcgtggtggtggatgtgtctcacgaggaccctgaagtgaagttcaattggtacgtggacggcgtggaagtgcacaacgccaagaccaagcctagagaggaacagtacggctccacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctagggaaccccaggtttacaccttgcctccatctcgggacgagctgaccaagaaccaggtgtccctgacctgtctcgtgaagggcttctacccctccgacatcgccgtggaatgggagtctaatggccagcctgagaacaactacaagacaacacctcctgtgctggactccgacggctcattcttcctgtactccgacctgaccgtggacaagtccagatggcagcagggcaacgtgttctcctgctccgtgatgcacgaggccctgcacaatcactacacccaggagagcctgtctctgtctcctgga

SEQ ID NO: 294 - DNA, 657 bp, synthetic construct
  source 1..657: mol_type = other DNA
gatgtggtgatgacccagtctcctctgtctctgcctgcaacacctggcgaccctgcctccatctcctgccgatcttcccagtccatcgtccactccaacggcaacacctacctggaatggtacctgcagaagcctggccagtccccaaagctgctgatctacaaggtgtccaacagattttctggcgtgcccgacagattctctggctctggctctggcaccgacttcaccctgaccatctctcgggtggacgccgaggatgtgggcatctactactgcttccagggcagccacgttccccctacctttggccctggcaccaagctggaaatcaagagaacagtggctgctccttccgtgttcatcttcccaccttccgacgagcagctgaagtccggcacagcttctgtcgtgtgcctgctgaacaacttctaccctcgggaagccaaggtgcagtggaaggtggacaatgccctgcagtccggcaactcccaagagtctgtgaccgagcaggactccaaggacagcacctacagcctgtcctccacactgaccctgtccaaggccgactacgagaagcacaaggtgtacgcctgcgaagtgacccatcagggcctgtctagccctgtgaccaagtctttcaaccggggcgagtgc

SEQ ID NO: 295 - DNA, 2139 bp, synthetic construct
  source 1..2139: mol_type = other DNA
gaggtgcagctggtggagtctggcggcggactggtgcaacctgggggcagcttgagactgtcttgctcggcttctggctttaccttcagctccttcggcatgcactgggtgcggcagacccccggcaagggcctggaatgggtcgcctacatctccagtggcagtagtacaatttactatggcgatactgtgaagggcagattcactatctctcgggacaacgccaaaaactccctgttcctgcagatgtcttccctcagagctgaggacacagctgtgtactattgcgccagagagggcggctactactacggcagatcctattacaccatggactactggggccaaggcaccacagtgaccgtcagcagtgcctccacaaaaggcccttctgtgttccctctggccccttcctccaaatctacatctggcggaaccgccgccctgggctgcctggtgaaggactacttccccgaacccgtgaccgtttcttggaactccggcgccctgacctccggcgtgcatacctttcctgccgtgctccaatccagcggcctgtacagcctgagctccgtggtcaccgtgccttcttccagcctgggcacccagacctacatctgcaatgtcaaccacaagcctagcaacaccaaggtggacaagaaggtggaacccaaatcctgcgacaagacccacacctgtcctccttgccctgctcctgaactgctgggcggcccttccgtgttcctgttcccacctaagcccaaggacaccctgatgatctctagaacccctgaggtgacctgcgtggtggtggacgtgtctcacgaggatcctgaagtgaagttcaactggtatgtggatggcgtggaagtgcacaacgctaagactaagcctcgggaagagcagtacggctctacctaccgggtggtgtctgtgctgacagtgctgcaccaggattggctgaacggcaaagagtacaagtgcaaggtttctaacaaggccctgcccgctcctatcgaaaagaccatctccaaggctaagggccagcctcgggagcctcaggtgtacaccctgcctccttcccggaaggagctgaccaagaaccaggtgagcctgacctgcctggtgaagggcttctacccctcagacatcgccgtggagtgggagtccaatggccagcctgagaacaactacaagacaacccctccagtgctgaagtctgacggatccttcttcctgtactccaagctgaccgtggataaatcaagatggcagcagggcaatgtgttctcctgcagcgtgatgcacgaggctctgcataatcactatacacagaagtccctgtccctgtctcctggcaagggcggaggtggctctgctgctgccgaagtgcagctgctggaatccggcggcggtctggtgcagcccggcggcagcctgaggctgagctgtgctgcctccggtttcaccttctccgactacgccatgtcctgggtgagacaagcccctggaaagggcctggagtgggtctccgccatcagatggcactcctcctggacctactacgccgactctgtgaagggccgcttcaccatcagcagagacaactctaagaacaccctgtacctgcagatgaactccctgagagccgaggataccgccgtgtactactgtgccaagatgaagtccaccagacattggatcgacgactggggccagggaacattggtcaccgtgtcttctggaggcggtggatctggtggtggcggatcaggtggaggcgggagcggaggcggcggtagtggtggaggaggctcttctgagctgacccaggacccggcggtgtccgtggccctgggccagaccgtgcggatcacctgtcagggcgactccttacggagctactacgcttcctggtaccagcagaaacccggacaggctcctgtgctggtgatctacggtaagaacaaccggccctctggcatcccagacagattttccggctcttcttccggcaacaccgcttccctaaccatcaccggcgctcaggccgaggacgaggccgattactactgccagtcttacgactcctcctcttaccactacgtgtttggcggcggcaccaagctgacggtcctg

SEQ ID NO: 296 - protein, 713 aa, synthetic construct
EVQLVESGGGLVQPGGSLRLSCSASGFTFSSFGMHWVRQTPGKGLEWVAYISSGSSTIYYGDTVKGRFTISRDNAKNSLFLQMSSLRAEDTAVYYCAREGGYYYGRSYYTMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRKELTKNQVSLTCLVKGFYPSDIAVEWESNGQPENNYKTTPPVLKSDGSFFLYSKLTVDKSRWQQGNVFSCSVMHEALHNHYTQKSLSLSPGKGGGGSAAAEVQLLESGGGLVQPGGSLRLSCAASGFTFSDYAMSWVRQAPGKGLEWVSAIRWHSSWTYYADSVKGRFTISRDNSKNTLYLQMNSLRAEDTAVYYCAKMKSTRHWIDDWGQGTLVTVSSGGGGSGGGGSGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCQSYDSSSYHYVFGGGTKLTVL

SEQ ID NO: 297 - DNA, 2139 bp, synthetic construct
  source 1..2139: mol_type = other DNA
gaggtgcaactcgtcgaatccggcggcggactggtgcagcctggcggatctctgagactatcctgcagcgcatctgggttcaccttcagcagctttggcatgcactgggtcagacagacccctggcaagggcctggaatgggtggcctacatcagctccggctcctctaccatctactacggcgacaccgtgaagggccggttcaccatctctagagataacgctaagaactccctgttcctgcagatgtcctctctgagagccgaagataccgccgtgtactactgtgctcgcgagggaggctactactacggcagaagctactacaccatggactactggggccaggggaccacagttacagtgtcttccgcctccaccaagggacctagtgtgttccctctggcgccatcttccaaaagcacctctggtggcaccgctgctctcggctgcctggtgaaggactactttccagagcctgtgactgtgtcctggaacagtggcgctctcacctccggagtgcataccttccctgctgtgctgcagtcttccggcttgtactccctgtccagcgtcgtcacggtgccttcctcctctctgggcacacagacttacatctgcaacgtgaaccacaagccttctaatacaaaggtggataagaaagtggaacctaagagctgcgacaaaacccacacctgtcccccttgccccgctcctgagctgctgggtggaccatccgtgtttctgttcccccccaagcctaaggacaccctgatgatctcgagaacccccgaggtgacctgcgtggtggtggacgtgtctcacgaagatcctgaagtgaaattcaactggtacgtggacggcgtggaagtgcacaacgccaaaacaaagcctcgggaagagcagtacggctccacctacagagtggtgtcagtactgacagttctgcaccaggactggctgaacggcaaagagtacaaatgcaaggtgagcaacaaggccctgcctgcccctatcgagaagaccatctccaaggccaagggccagcccagagagccacaggtgtataccctgcccccttcccggaaggaactgaccaagaaccaggtcagtctgacctgcctggtgaagggcttctatccctccgacatcgccgtggagtgggagtctaacggccagcctgagaacaactacaagaccacccctcctgtgctgaagtccgatggctccttcttcctgtacagcaagctgacagtggacaagtctcggtggcagcagggcaatgtgttctcctgctccgtgatgcacgaggccctgcacaaccattacacccagaaatccctgtctctgtcccctggcaagggcggaggtggctctgccgccgccgaggtgcagctgctggagtccggcggtggcctggtgcagcctggcgggtctctgcggctgtcttgtgccgcttctggcttcaccttctccgactacgccatgagctgggtgagacaggcccctggcaagggcctggaatgggtgagtgccatccggtggcactctggctggacctattacgccgactccgtgaagggcagatttaccatctcccgggacaactccaagaacaccctgtacctgcagatgaactctctcagggctgaggacaccgctgtgtactactgcgccaagatgaaatctacaagacactggattgacgattggggacaaggcaccctggtgaccgtctcctccggaggcggtggatctggtggtggcggatcaggtggaggcgggagcggaggcggcggtagtggtggaggaggctctagcgagctgacccaggatcctgctgtgtccgtggctctgggccaaaccgtgcggatcacctgtcaaggcgactctctgcgttcttactacgcctcctggtatcagcagaagcccggccaggctcccgtgctggtgatctacggcaagaacaaccggccttctggcatccctgacagattctctggatcttcatctggcaacaccgcctccctgacaatcaccggcgctcaggccgaggacgaggctgattactactgccagtcctacgactcttcttcctaccactacgtgttcggcggaggcaccaagctgaccgtcctg

SEQ ID NO: 298 - protein, 713 aa, synthetic construct
EVQLVESGGGLVQPGGSLRLSCSASGFTFSSFGMHWVRQTPGKGLEWVAYISSGSSTIYYGDTVKGRFTISRDNAKNSLFLQMSSLRAEDTAVYYCAREGGYYYGRSYYTMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRKELTKNQVSLTCLVKGFYPSDIAVEWESNGQPENNYKTTPPVLKSDGSFFLYSKLTVDKSRWQQGNVFSCSVMHEALHNHYTQKSLSLSPGKGGGGSAAAEVQLLESGGGLVQPGGSLRLSCAASGFTFSDYAMSWVRQAPGKGLEWVSAIRWHSGWTYYADSVKGRFTISRDNSKNTLYLQMNSLRAEDTAVYYCAKMKSTRHWIDDWGQGTLVTVSSGGGGSGGGGSGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCQSYDSSSYHYVFGGGTKLTVL

SEQ ID NO: 299 - DNA, 2139 bp, synthetic construct
  source 1..2139: mol_type = other DNA
gaggtgcagctggtggagtctggcggcggtctggtgcaacctggcggatcactgcgcttaagttgctccgcatccggcttcactttctcctcctttggcatgcattgggtcagacagacccctggaaaaggcctggaatgggtggcctacatctcttccggctcttccaccatctattacggcgataccgttaagggcagattcactatctcccgggataacgccaagaactccctgtttctgcagatgagcagcctgagagccgaggacacagctgtgtactactgcgccagagagggcggctactactatggcagatcctactacaccatggactactggggacaaggcaccaccgtgaccgtgtcatccgcttccaccaaaggaccttccgtgtttcctctggccccttctagcaagtctacttccggcggcaccgccgccctgggctgcctcgtcaaggactacttccctgagcccgtgacagtgtcttggaactccggcgctctgacctccggcgtgcacacctttcccgccgtgctgcagtctagcggcctgtactctctgtcttccgtggtgaccgtgccctcatcttcactgggcacccagacctacatctgcaacgtgaaccacaagccctccaacaccaaggtggataagaaagtggaacccaagtcctgcgacaagacccacacctgtcctccttgccctgccccagagctgctgggcggccctagcgttttcctgttcccacctaagcctaaggataccctgatgatctcccggaccccggaagtgacctgcgtggtggtggacgtgtctcacgaggaccctgaagtgaaattcaactggtatgtggacggcgttgaagtccacaacgctaaaaccaagcctcgcgaggaacagtacggatccacctacagagtggtgtctgtcctgaccgtgctgcaccaggactggctgaacggcaaagagtacaagtgcaaggtgtccaacaaggccctgcctgctcctatcgagaagaccatctccaaggctaagggccagcctagagagcctcaggtgtacaccctgcctccatctcgtaaggaactgacaaagaaccaggtttccctgacctgcctagtgaagggcttctaccccagcgacatcgctgtcgagtgggagtccaatggccagcctgagaacaactacaagacaacacctcccgtgctgaagtccgatggctcgttcttcctgtactccaagctgacagtagacaagagccggtggcaacagggcaacgtgttctcctgctccgtgatgcacgaggctctgcataatcactacacacagaaatctctgtctctgtctccaggcaaaggcggaggtggctctgccgctgctgaggtgcagctgctggaaagcggaggcggactggtgcagcccggcggctctctgcggctgtcctgtgctgcttctgggttcaccttctccgactacgccatgtcctgggtgcggcaggcccctggcaagtgcctggaatgggtgtctgccatcagatggcactcctcctggacctactatgccgactctgtgaagggacggttcaccatttctagagacaattctaagaacaccctgtacctgcagatgaactccctgagagctgaagacaccgctgtgtactactgtgccaagatgaagtctaccagacactggatcgacgattggggccaaggcacactggttaccgtgtcctctggaggcggtggatctggtggtggcggatcaggtggaggcgggagcggaggcggcggtagtggtggaggaggctcttccgagctgacccaggatcctgccgtgtccgtggctctcggccaaaccgtacggatcacctgtcagggcgactccctgcggtcttactacgcctcttggtaccagcagaagcctggccaggccccagtgctggtgatctacggcaagaacaaccggccttctggcatccctgacagattcagcggctccagttctggcaacaccgccagcctgaccatcaccggagcccaggccgaggacgaggccgattactactgccagtcctacgactcctcctcctaccactacgtgttcggctgtggcaccaagctgacagtgctg

SEQ ID NO: 300 - protein, 713 aa, synthetic construct
EVQLVESGGGLVQPGGSLRLSCSASGFTFSSFGMHWVRQTPGKGLEWVAYISSGSSTIYYGDTVKGRFTISRDNAKNSLFLQMSSLRAEDTAVYYCAREGGYYYGRSYYTMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRKELTKNQVSLTCLVKGFYPSDIAVEWESNGQPENNYKTTPPVLKSDGSFFLYSKLTVDKSRWQQGNVFSCSVMHEALHNHYTQKSLSLSPGKGGGGSAAAEVQLLESGGGLVQPGGSLRLSCAASGFTFSDYAMSWVRQAPGKCLEWVSAIRWHSSWTYYADSVKGRFTISRDNSKNTLYLQMNSLRAEDTAVYYCAKMKSTRHWIDDWGQGTLVTVSSGGGGSGGGGSGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCQSYDSSSYHYVFGCGTKLTVL

SEQ ID NO: 301 - DNA, 2139 bp, synthetic construct
  source 1..2139: mol_type = other DNA
gaagtgcagctggtcgagagcggcggcggcctggtccagcccggcggctctctgcgcctgtcttgcagtgcttctggcttcaccttctctagcttcggcatgcactgggtgcggcagaccccgggaaagggcttggaatgggtggcctacatctcctcaggatctagcaccatctactatggcgacaccgtgaaaggcaggtttaccatctccagagacaacgccaagaactcactctttctgcaaatgtcctccctgcgggccgaggataccgctgtgtactactgcgccagagaaggcggctactactatggcagatcttactacaccatggactactggggccagggcaccaccgtgaccgtgagctctgcctctaccaaaggccctagcgtgttccctctggctccatcctctaagtccacttccggcggcaccgccgctctgggctgtctggtgaaggattacttccctgaacctgtgacagtgtcttggaactccggcgccctgacctctggcgtgcacacctttcccgccgtgctgcaatcttccggcctgtacagcctgtcctctgtcgtgacggtgccttccagctctctgggaacccagacctacatctgcaacgtgaaccacaagccctctaataccaaggtggacaagaaggtggaacccaagtcttgcgacaagacacacacatgtcctccatgccctgctcccgaactgctgggaggcccctcggtgttcctgttcccacccaaacctaaggacaccctgatgatctcccggacccctgaggtgacctgcgtggtggtggatgtgtctcacgaggatcctgaagtcaagttcaactggtacgtggatggcgtggaagtgcacaatgccaagaccaaacctcgggaagagcagtacggctccacctatagagtggtctctgtcctgaccgtgctgcatcaggattggctgaacggcaaagagtacaagtgcaaggtgtccaacaaggccctgcctgctcccatcgagaagaccatcagtaaagccaagggccagcctagagagccccaggtgtacaccctgccaccttcccggaaggagctgaccaagaaccaggtttctcttacctgcctggtgaagggcttctacccttccgacatcgccgtcgagtgggagtccaacggccaacctgagaacaactacaagaccacacctcctgtgctgaagtccgacggctccttcttcctgtactccaagctgaccgtggacaagtctagatggcagcaaggcaacgtgttcagctgctccgtgatgcacgaggctctgcacaaccactacacacagaagtctctgtctctgtctcctggcaagggcggaggtggctctgctgctgccgaggtgcagctgctagaatccggaggcggactggtgcagcctggcggtagcctgcgcctgtcctgtgctgcctctggctttaccttctccgactacgccatgtcctgggtcagacaggcccctggcaagtgcctggagtgggtgtctgccatccggtggcactccggctggacctattacgctgactctgtcaagggccggttcacaatttcacgggacaattccaagaacaccctgtacctgcagatgaactccctgagagccgaggataccgctgtgtactactgtgctaagatgaagtccacaagacattggatcgacgactggggacagggaaccttagtgaccgtgtcttcgggaggcggtggatctggtggtggcggatcaggtggaggcgggagcggaggcggcggtagtggtggaggaggctcttcggagctgacccaggaccctgccgtgagtgtggccctgggccagacagtgcggatcacctgtcagggcgactccctgagatcctactatgcttcctggtaccagcagaagcctggccaggccccagtgctcgtgatctacggcaaaaacaacagacctagcggcatccccgacagattctccggatcctcctctggcaacaccgcctctctgaccatcaccggcgctcaggctgaggacgaggcagactactactgccagtcctacgattcctcttcctaccactacgtgttcggctgcggcaccaagctgaccgtgctg

SEQ ID NO: 302 - protein, 713 aa, synthetic construct
EVQLVESGGGLVQPGGSLRLSCSASGFTFSSFGMHWVRQTPGKGLEWVAYISSGSSTIYYGDTVKGRFTISRDNAKNSLFLQMSSLRAEDTAVYYCAREGGYYYGRSYYTMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRKELTKNQVSLTCLVKGFYPSDIAVEWESNGQPENNYKTTPPVLKSDGSFFLYSKLTVDKSRWQQGNVFSCSVMHEALHNHYTQKSLSLSPGKGGGGSAAAEVQLLESGGGLVQPGGSLRLSCAASGFTFSDYAMSWVRQAPGKCLEWVSAIRWHSGWTYYADSVKGRFTISRDNSKNTLYLQMNSLRAEDTAVYYCAKMKSTRHWIDDWGQGTLVTVSSGGGGSGGGGSGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCQSYDSSSYHYVFGCGTKLTVL

SEQ ID NO: 303 - DNA, 1359 bp, synthetic construct
  source 1..1359: mol_type = other DNA
gaggtgcagctggtggaatctggcggaggattggttcagcctggcggctctctgagactgtcctgttctgcctccggcttcaccttctccagcttcggcatgcactgggtccgacagacccctggcaaaggattggagtgggtcgcctacatctcctccggctcctccaccatctactacggcgataccgtgaagggcagattcaccatctctcgggacaacgccaagaactccctgttcctgcagatgtccagcctgagagccgaggataccgccgtgtactactgtgctagagaaggcggctactactatggccggtcctactacaccatggactactggggccagggcaccacagtgacagtgtcctctgctaagaccaccgctcctagcgtgtaccctctggctcctgtgtgtggcgataccaccggatcctctgtgaccctgggatgtctggtcaagggctacttccccgagcctgtgacactgacctggaactccggatctctgtcctccggcgtgcacacatttccagccgtgctgcagtccgacctgtacaccctgtctagctccgtgaccgtgacctcctctacctggcctagccagtccatcacctgtaacgtggcccatcctgcctccagcaccaaggtggacaagaagatcgagcctcggggccctaccatcaagccttgtcctccatgcaagtgccccgctcctaatctgctcggaggcccttccgtgttcatcttcccacctaagatcaaggacgtgctgatgatctccctgtctcctatcgtgacctgcgtggtggtggacgtgtccgaggatgatcctgacgtgcagatcagttggttcgtgaacaacgtggaagtgcacaccgctcagacccagacacacagagaggactacaacagcaccctgagagtggtgtctgccctgcctatccagcatcaggattggatgtccggcaaagaattcaagtgcaaagtgaacaacaaggacctgcctgctccaatcgagcggaccatctctaagcctaagggctctgtcagggcccctcaggtgtacgttttgcctccacctgaggaagagatgaccaagaaacaagtgaccctgacatgcatggtcaccgacttcatgcccgaggacatctacgtggaatggaccaacaacggcaagaccgagctgaactacaagaacaccgagccagtgctggactccgacggctcctacttcatgtactctgacctgcgcgtcgagaagaagaactgggtcgagagaaactcctactcctgctccgtggtgcatgagggcctgcacaatcaccacaccaccgagtccttcagcagaacccctgga

SEQ ID NO: 304 - protein, 453 aa, synthetic construct
EVQLVESGGGLVQPGGSLRLSCSASGFTFSSFGMHWVRQTPGKGLEWVAYISSGSSTIYYGDTVKGRFTISRDNAKNSLFLQMSSLRAEDTAVYYCAREGGYYYGRSYYTMDYWGQGTTVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSDLRVEKKNWVERNSYSCSVVHEGLHNHHTTESFSRTPG

SEQ ID NO: 305 - DNA, 2109 bp, synthetic construct
  source 1..2109: mol_type = other DNA
gaggtgcagctggtggaatctggcggaggattggttcagcctggcggctctctgagactgtcctgttctgcctccggcttcaccttctccagcttcggcatgcactgggtccgacagacccctggcaaaggattggagtgggtcgcctacatctcctccggctcctccaccatctactacggcgataccgtgaagggcagattcaccatctctcgggacaacgccaagaactccctgttcctgcagatgtccagcctgagagccgaggataccgccgtgtactactgtgctagagaaggcggctactactatggccggtcctactacaccatggactactggggccagggcaccacagtgacagtgtcctctgctaagaccaccgctcctagcgtgtaccctctggctcctgtgtgtggcgataccaccggatcctctgtgaccctgggatgtctggtcaagggctacttccccgagcctgtgacactgacctggaactccggatctctgtcctccggcgtgcacacatttccagccgtgctgcagtccgacctgtacaccctgtctagctccgtgaccgtgacctcctctacctggcctagccagtccatcacctgtaacgtggcccatcctgcctccagcaccaaggtggacaagaagatcgagcctcggggccctaccatcaagccttgtcctccatgcaagtgccccgctcctaatctgctcggaggcccttccgtgttcatcttcccacctaagatcaaggacgtgctgatgatctccctgtctcctatcgtgacctgcgtggtggtggacgtgtccgaggatgatcctgacgtgcagatcagttggttcgtgaacaacgtggaagtgcacaccgctcagacccagacacacagagaggactacaacagcaccctgagagtggtgtctgccctgcctatccagcatcaggattggatgtccggcaaagaattcaagtgcaaagtgaacaacaaggacctgcctgctccaatcgagcggaccatctctaagcctaagggctctgtcagggcccctcaggtgtacgttttgcctccacctgagaaagagatgaccaagaagcaagtcaccctgacctgcatggtcaccgacttcatgcccgaggacatctacgtggaatggaccaacaacggcaagaccgagctgaactacaagaacaccgagccagtgctgaagtctgacggctcctacttcatgtactccaagctgcgcgtcgagaagaagaactgggtcgagagaaactcctactcctgctccgtggtgcatgagggcctgcacaatcaccacaccaccaagtccttcagcagaacacccggaaaaggcggcggtggatctgctgctgctgaagtgcagttgctggaaagcggaggtggacttgtgcagccaggtggaagcctgagactgagctgtgccgcttctggctttaccttcagcgactacgccatgtcctgggtccgccaggcacctggaaaatgtctggaatgggtgtccgctatccggtggcactcctcctggacctactacgccgattctgtgaaaggccggtttaccatcagccgggataactctaagaataccctgtacctccagatgaactccctccgcgctgaggacaccgctgtgtattattgcgccaagatgaagtctacccggcactggatcgacgattggggacagggaacactggtcaccgtttctagcggcggaggcggatctggtggtggcggtagtggtggcggcggatcttctgaactgacccaggatccagctgtgtccgtggctctgggacagacagtgcggattacatgccagggcgactccctgagatcttactacgcctcctggtatcagcagaagcccggccaagctcctgtgctggtcatctacggcaagaacaaccggcctagcggcatccctgatcggttctctggatcctccagcggcaataccgccagcctgacaattactggcgcccaggctgaggacgaggccgactactactgccagtcctacgactcctcctcctaccactacgtgttcggctgtggcaccaagctgactgtgttg

SEQ ID NO: 306 - protein, 703 aa, synthetic construct
EVQLVESGGGLVQPGGSLRLSCSASGFTFSSFGMHWVRQTPGKGLEWVAYISSGSSTIYYGDTVKGRFTISRDNAKNSLFLQMSSLRAEDTAVYYCAREGGYYYGRSYYTMDYWGQGTTVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEKEMTKKQVTLTCMVTDFMPEDIYVEWTNNGKTELNYKNTEPVLKSDGSYFMYSKLRVEKKNWVERNSYSCSVVHEGLHNHHTTKSFSRTPGKGGGGSAAAEVQLLESGGGLVQPGGSLRLSCAASGFTFSDYAMSWVRQAPGKCLEWVSAIRWHSSWTYYADSVKGRFTISRDNSKNTLYLQMNSLRAEDTAVYYCAKMKSTRHWIDDWGQGTLVTVSSGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCQSYDSSSYHYVFGCGTKLTVL

SEQ ID NO: 307 - DNA, 657 bp, synthetic construct
  source 1..657: mol_type = other DNA
gacgtggtcatgacacagagccctctgtctctgcctgctacacctggcgatcctgcctccatctcctgcagatcctctcagtccatcgtgcactccaacggcaacacctacctggaatggtatctgcagaagcccggccagtctcctaagctgctgatctacaaggtgtccaaccggttctctggcgtgcccgacagattttccggctctggctctggcaccgacttcaccctgaccatctctagagtggacgccgaggacgtgggcatctactactgcttccaaggctcccatgtgcctccaacctttggacctggcaccaagctggaaatcaagcgggctgatgctgcccctaccgtgtctatcttcccacctagctctgagcagctgacctctggcggagcttctgtcgtgtgcttcctgaacaacttctaccccaaggacatcaacgtgaagtggaagatcgacggctccgagagacagaacggcgtgctgaactcttggaccgaccaggactccaaggacagcacctactccatgtcctccacactgaccctgacaaaggacgagtacgagcggcacaactcctatacctgcgaggctacccacaagacctccacctctccaatcgtgaagtccttcaaccggaacgagtgc

SEQ ID NO: 308 - DNA, 2109 bp, synthetic construct
  source 1..2109: mol_type = other DNA
gaggtgcagctggtggaatctggcggaggattggttcagcctggcggctctctgagactgtcctgttctgcctccggcttcaccttctccagcttcggcatgcactgggtccgacagacccctggcaaaggattggagtgggtcgcctacatctcctccggctcctccaccatctactacggcgataccgtgaagggcagattcaccatctctcgggacaacgccaagaactccctgttcctgcagatgtccagcctgagagccgaggataccgccgtgtactactgtgctagagaaggcggctactactatggccggtcctactacaccatggactactggggccagggcaccacagtgacagtgtcctctgctaagaccaccgctcctagcgtgtaccctctggctcctgtgtgtggcgataccaccggatcctctgtgaccctgggatgtctggtcaagggctacttccccgagcctgtgacactgacctggaactccggatctctgtcctccggcgtgcacacatttccagccgtgctgcagtccgacctgtacaccctgtctagctccgtgaccgtgacctcctctacctggcctagccagtccatcacctgtaacgtggcccatcctgcctccagcaccaaggtggacaagaagatcgagcctcggggccctaccatcaagccttgtcctccatgcaagtgccccgctcctaatctgctcggaggcccttccgtgttcatcttcccacctaagatcaaggacgtgctgatgatctccctgtctcctatcgtgacctgcgtggtggtggacgtgtccgaggatgatcctgacgtgcagatcagttggttcgtgaacaacgtggaagtgcacaccgctcagacccagacacacagagaggactacaacagcaccctgagagtggtgtctgccctgcctatccagcatcaggattggatgtccggcaaagaattcaagtgcaaagtgaacaacaaggacctgcctgctccaatcgagcggaccatctctaagcctaagggctctgtcagggcccctcaggtgtacgttttgcctccacctgagaaagagatgaccaagaagcaagtcaccctgacctgcatggtcaccgacttcatgcccgaggacatctacgtggaatggaccaacaacggcaagaccgagctgaactacaagaacaccgagccagtgctgaagtctgacggctcctacttcatgtactccaagctgcgcgtcgagaagaagaactgggtcgagagaaactcctactcctgctccgtggtgcatgagggcctgcacaatcaccacaccaccaagtccttcagcagaacacccggaaaaggcggcggtggatctgctgctgctgaagtgcagttgctggaaagcggaggtggacttgtgcagccaggtggaagcctgagactgagctgtgccgcttctggctttaccttcagcgactacgccatgtcctgggtccgccaggcacctggaaaatgtctggaatgggtgtccgccatccggtggcattctggctggacctactacgccgactctgtgaaaggccggttcaccatcagcagagacaactctaagaataccctgtatctccagatgaactccctccgcgctgaggacaccgctgtgtattattgcgccaagatgaagtctacccggcactggatcgacgattggggacagggaacactggtcaccgtttctagcggcggaggcggatctggtggtggcggtagtggtggcggcggatcttctgaactgacccaggatccagctgtgtccgtggctctgggacagacagtgcggattacatgccagggcgactccctgagatcttactacgcctcctggtatcagcagaagcccggccaagctcctgtgctggtcatctacggcaagaacaaccggcctagcggcatccctgatcggttctctggatcctccagcggcaataccgccagcctgacaattactggcgcccaggctgaggacgaggccgactactactgccagtcctacgactcctcctcctaccactacgtgttcggctgtggcaccaagctgactgtgttg

(19) United States
(12) Patent Application Publication
(10) Pub. No.: US 2026/0297208 A1
(43) Pub. Date: Oct. 1, 2026

(54) BISPECIFIC ANTIBODY

(71) Applicant: Eisai R&D Management Co., Ltd., Tokyo (JP)

(72) Inventors: Naoto WATANABE, Ibaraki (JP); Risa TAKAHASHI, Ibaraki (JP); Eiji INOUE, Ibaraki (JP); Mami GOMIBUCHI, Ibaraki (JP); Masashi SHIMIZU, Ibaraki (JP); Yosuke WATANABE, Ibaraki (JP); Hiroki KAWAHARA, Hyogo (JP); Yoko IDA, Hyogo (JP); Yohei MUKAI, Osaka (JP); Yuta ISHIZUKA, Hyogo (JP); Yoshihisa ARITA, Hyogo (JP); Tomoya NAKATANI, Hyogo (JP); Eri NISHIOKA, Hyogo (JP)

(21) Appl. No.: 19/570,929

(22) Filed: Mar. 18, 2026

Related U.S. Application Data
(60) Provisional application No. 63/775,484, filed on Mar. 21, 2025.
(60) Provisional application No. 63/809,330, filed on May 20, 2025.
(60) Provisional application No. 63/858,306, filed on Aug. 6, 2025.
(60) Provisional application No. 63/930,988, filed on Dec. 4, 2025.

(51) Int. Cl. C07K 16/28; A61P 25/28; C07K 16/18

(52) U.S. Cl. CPC C07K 16/2881; A61P 25/28; C07K 16/18; C07K 2317/31; C07K 2317/622

(57) ABSTRACT

A bispecific antibody comprising a first antigen-binding polypeptide binding to amyloid beta (Aβ) and a second antigen-binding polypeptide binding to a human transferrin receptor (TfR), wherein: (i) the first antigen-binding polypeptide comprises a first heavy chain, a second heavy chain, and two light chains, the first heavy chain and the second heavy chain each comprise a heavy chain variable region consisting of the amino acid sequence set forth in SEQ ID NO: 238, the two light chains each comprise a light chain variable region consisting of the amino acid sequence set forth in SEQ ID NO: 239; (ii) the second antigen-binding polypeptide comprises a heavy chain variable region (VH) comprising a HCDR1 consisting of the amino acid sequence set forth in SEQ ID NO: 2, a HCDR2 consisting of the amino acid sequence set forth in SEQ ID NO: 8 or SEQ ID NO: 13, and a HCDR3 consisting of the amino acid sequence set forth in SEQ ID NO: 3, and a light chain variable region (VL) comprising a LCDR1 consisting of the amino acid sequence set forth in SEQ ID NO: 4, a LCDR2 consisting of the amino acid sequence set forth in SEQ ID NO: 5, and a LCDR3 consisting of the amino acid sequence set forth in SEQ ID NO: 6; and (iii) the second antigen-binding polypeptide is connected to the C-terminus of a constant region of the second heavy chain of the first antigen-binding polypeptide.

### CLAIM OF PRIORITY

This application claims priority to U.S. Patent Application Ser. No. 63/775,484, filed on Mar. 21, 2025, U.S. Patent Application Ser. No. 63/809,330, filed on May 20, 2025, U.S. Patent Application Ser. No. 63/858,306, filed on Aug. 6, 2025, and U.S. Patent Application Ser. No. 63/930,988, filed on Dec. 4, 2025, the entire contents of which are hereby incorporated by reference.

### SEQUENCE LISTING

This application contains a Sequence Listing that has been submitted electronically as an XML file named FP25-1466-00US-ECL_SEQL.xml. The XML file, created on Mar. 5, 2026, is 501,877 bytes in size. The material in the XML file is hereby incorporated by reference in its entirety.

### TECHNICAL FIELD

The present invention relates to a bispecific antibody, and in particular to a bispecific antibody comprising a first antigen-binding polypeptide binding to amyloid beta (Aβ) and a second antigen-binding polypeptide binding to a human transferrin receptor (TfR).

### BACKGROUND

The blood-brain barrier (BBB) blocks drug transport of hydrophilic drugs to the brain, and in particular, is the biggest barrier to the brain delivery of biopharmaceuticals. Receptor-mediated transcytosis (RMT) is a route capable of transporting high-molecular-weight proteins, and thus, it has attracted the most attention as a technique for permeating biopharmaceuticals across the blood-brain barrier (Non Patent Literature 1).

Among RMT, the transferrin receptor (TfR)-mediated brain delivery using an anti-TfR antibody has been attracting attention. On the other hand, the brain delivery of antibody therapeutics has been reported that it impairs the prolonged blood half-life of an antibody (Non Patent Literature 2). Accordingly, it is a challenge to find a way to increase the amount transferred into the brain while suppressing the rapid disappearance from the blood.

To address the above challenge, for example, a technique for brain delivery using an anti-TfR antibody called a brain shuttle antibody (Patent Literature 1, Non Patent Literature 3, and the like), and a technique for brain delivery using an antibody transport vehicle (ATV) by an anti-TfR mutant Fc domain (Non Patent Literature 4) have been proposed. In addition to these, techniques for brain delivery such as a low affinity anti-TfR antibody (Patent Literature 2) and a pH-dependent anti-TfR antibody in which the affinity for TfR changes in a pH-dependent manner (Patent Literature 3, and Non Patent Literature 5) have also been proposed.

On the other hand, the amyloid cascade hypothesis is believed to be the main cause of Alzheimer's disease (AD), in which amyloid β (Aβ) abnormally accumulates outside the neural cells, and then, neurofibrillary tangles of tau are generated to cause disorders of neural cells, leading to the development of diseases. Anti-amyloid β (Aβ) antibodies are believed to be useful for treating a disease associated with amyloid β (Aβ) deposits, and especially Alzheimer's disease (AD). As the anti-amyloid β (Aβ) antibody, the one described in Patent Literature 4 is known.

It is known that antibodies against amyloid β (Aβ) may cause amyloid-related imaging abnormalities (ARIA), and ARIA is characterized as ARIA with cerebral edema or sulcal effusion (ARIA-E) and ARIA with superficial hemosiderosis (ARIA-H), and observed by MRI. In particular, it is known that the risk of ARIA is high in APOE &4 genotype carriers (Non Patent Literatures 6 and 7). It is known that, in Alzheimer's disease, cerebral amyloid angiopathy (CAA) is present in about 80% of the patients, and it is suggested that administered anti-Aβ antibodies binding to CAA causes immunoreaction to be evoked at blood vessel spots, and the blood-brain barrier (BBB) being reduced in function or broken down causes ARIA (Non Patent Literature 8). Further, it is confirmed that, in a human brain to which anti-AB antibodies are administered, infiltration of immune cells into the blood vessel periphery occurs (Non Patent Literature 9). More specifically, it is suggested that the anti-Aβ antibodies bound to CAA bind to complement C1q, and the activation of the complement cascade thus occurs, causing ARIA (Non Patent Literature 10).

It is said that antibodies have five major roles of neutralizing effect, complement-dependent cytotoxicity (CDC) activity, antibody-dependent cellular cytotoxicity (ADCC) activity, antibody-dependent cellular phagocytosis (ADCP) activity, and agonist activity. Among these, it is commonly known that CDC occurs when the Fc region of the antibody binds to C1q, and ADCC occurs when the Fc region of the antibody binds to an Fcγ receptor expressed in immune cells (effector cells) such as natural killer (NK) cells and macrophages.

### CITATION LIST

### Patent Literature

Patent Literature 1: WO2017/055540
Patent Literature 2: WO2012/075037
Patent Literature 3: WO2012/143379
Patent Literature 4: WO2016/005466

### Non Patent Literature

Non Patent Literature 1: Nga Bien-Ly et al., J. Exp. Med. 211 (2), 233-244 (2014)
Non Patent Literature 2: Y. Joy Yu et al., Sci. Transl. Med. 6 (261) 261ra154 (2014).
Non Patent Literature 3: Felix Weber et al., Cell Reports 22, 149-162 (2018)
Non Patent Literature 4: Mihalis S. Kariolis et al., Sci. Transl. Med. 12, eaay1359 (2020)
Non Patent Literature 5: Hadassah Sade et al., Plos One 2014, 9 (4), e96340
Non Patent Literature 6: Reisa A. Sperling et al., Alzheimer's & Dementia, Volume 7, Issue 4, July 2011, Pages 367-385; 10.1016/j.jalz.2011.05.2351
Non Patent Literature 7: Jacopo C DiFrancesco et al., Front Neurol. 2015 Sep. 25:6:207. doi: 10.3389/fneur.2015.00207. eCollection 2015.
Non Patent Literature 8: Roxana Aldea et al., Alzheimers Dement (NY). 2022 Jun. 6; 8 (1): e12306
Non Patent Literature 9: Elena Solopova et al., Nat Commun. 2023 Dec. 12; 14 (1): 8220
Non Patent Literature 10: Alzforum website, Madolyn Bowman Rogers, “Is ARIA an Inflammatory Reaction to Vascular Amyloid?”, searched on Mar. 14, 2025, Internet <https://www.alzforum.org/news/conference-coverage/aria-inflammatory-reaction-vascular-amyloid>

### SUMMARY

### Technical Problem

An object of the present invention is to provide a bispecific antibody comprising a first antigen-binding polypeptide binding to amyloid beta (Aβ) and a second antigen-binding polypeptide binding to a human transferrin receptor (TfR), the bispecific antibody having a high brain penetrability.

### Solution to Problem

The present inventors have obtained, by screening using human TfR proteins and a fully synthetic human antibody phage library, an anti-human transferrin receptor antibody that specifically binds to human TfR, wherein the binding has a certain pH-dependency, or an antigen-binding fragment thereof, and found that the antibody or an antigen-binding fragment thereof has a high brain penetrability.

That is, the present invention relates to, for example, the following respective inventions.
[1] A bispecific antibody comprising a first antigen-binding polypeptide binding to amyloid beta (Aβ) and a second antigen-binding polypeptide binding to a human transferrin receptor (TfR), wherein:
(i) the first antigen-binding polypeptide comprises a first heavy chain, a second heavy chain, and two light chains,
   the first heavy chain and the second heavy chain each comprise a heavy chain variable region (VH) consisting of the amino acid sequence set forth in SEQ ID NO: 238,
   the two light chains each comprise a light chain variable region (VL) consisting of the amino acid sequence set forth in SEQ ID NO: 239;
(ii) the second antigen-binding polypeptide comprises a heavy chain variable region comprising a heavy chain CDR1 (HCDR1) consisting of the amino acid sequence set forth in SEQ ID NO: 2, a heavy chain CDR2 (HCDR2) consisting of the amino acid sequence set forth in SEQ ID NO: 8 or SEQ ID NO: 13, and a heavy chain CDR3 (HCDR3) consisting of the amino acid sequence set forth in SEQ ID NO: 3, and a light chain variable region comprising a light chain CDR1 (LCDR1) consisting of the amino acid sequence set forth in SEQ ID NO: 4, a light chain CDR2 (LCDR2) consisting of the amino acid sequence set forth in SEQ ID NO: 5, and a light chain CDR3 (LCDR3) consisting of the amino acid sequence set forth in SEQ ID NO: 6; and
(iii) the second antigen-binding polypeptide is connected to the C-terminus of a constant region of the second heavy chain of the first antigen-binding polypeptide.
[2] A bispecific antibody comprising a first antigen-binding polypeptide binding to amyloid beta (Aβ) and a second antigen-binding polypeptide binding to a human transferrin receptor (TfR), wherein:
(i) the first antigen-binding polypeptide comprises a first heavy chain, a second heavy chain, and two light chains,
   the first heavy chain and the second heavy chain each comprise a heavy chain CDR1 (HCDR1) consisting of the amino acid sequence set forth in SEQ ID NO: 232, a heavy chain CDR2 (HCDR2) consisting of the amino acid sequence set forth in SEQ ID NO: 233, and a heavy chain CDR3 (HCDR3) consisting of the amino acid sequence set forth in SEQ ID NO: 234,
   the two light chains each comprise a light chain CDR1 (LCDR1) consisting of the amino acid sequence set forth in SEQ ID NO: 235, a light chain CDR2 (LCDR2) consisting of the amino acid sequence set forth in SEQ ID NO: 236, and a light chain CDR3 (LCDR3) consisting of the amino acid sequence set forth in SEQ ID NO: 237;
(ii) the second antigen-binding polypeptide comprises a heavy chain variable region (VH) comprising HCDR1 consisting of the amino acid sequence set forth in SEQ ID NO: 2, HCDR2 consisting of the amino acid sequence set forth in SEQ ID NO: 8 or SEQ ID NO: 13, and HCDR3 consisting of the amino acid sequence set forth in SEQ ID NO: 3, and a light chain variable region (VL) comprising LCDR1 consisting of the amino acid sequence set forth in SEQ ID NO: 4, LCDR2 consisting of the amino acid sequence set forth in SEQ ID NO: 5, and LCDR3 consisting of the amino acid sequence set forth in SEQ ID NO: 6; and
(iii) the second antigen-binding polypeptide is connected to the C-terminus of the constant region of the second heavy chain of the first antigen-binding polypeptide.
[3] A bispecific antibody comprising a first antigen-binding polypeptide binding to amyloid beta (Aβ) and a second antigen-binding polypeptide binding to a human transferrin receptor (TfR), wherein:
(i) the first antigen-binding polypeptide comprises a first heavy chain, a second heavy chain, and two light chains,
   the first heavy chain and the second heavy chain each comprise a heavy chain CDR1 (HCDR1) consisting of the amino acid sequence set forth in SEQ ID NO: 232, a heavy chain CDR2 (HCDR2) consisting of the amino acid sequence set forth in SEQ ID NO: 233, and a heavy chain CDR3 (HCDR3) consisting of the amino acid sequence set forth in SEQ ID NO: 234,
   the two light chains each comprise a light chain CDR1 (LCDR1) consisting of the amino acid sequence set forth in SEQ ID NO: 235, a light chain CDR2 (LCDR2) consisting of the amino acid sequence set forth in SEQ ID NO: 236, and a light chain CDR3 (LCDR3) consisting of the amino acid sequence set forth in SEQ ID NO: 237;
(ii) the second antigen-binding polypeptide comprises a heavy chain variable region (VH) comprising HCDR1 consisting of the amino acid sequence set forth in SEQ ID NO: 2, HCDR2 consisting of the amino acid sequence set forth in SEQ ID NO: 8 or SEQ ID NO: 13, and HCDR3 consisting of the amino acid sequence set forth in SEQ ID NO: 3, and a light chain variable region (VL) comprising LCDR1 consisting of the amino acid sequence set forth in SEQ ID NO: 4, LCDR2 consisting of the amino acid sequence set forth in SEQ ID NO: 5, and LCDR3 consisting of the amino acid sequence set forth in SEQ ID NO: 6; and
(iii) the second antigen-binding polypeptide is connected to the C-terminus of the constant region of the second heavy chain of the first antigen-binding polypeptide,
(iv) provided that those wherein a first heavy chain and a second heavy chain each comprise a heavy chain variable region consisting of the amino acid sequence set forth in SEQ ID NO: 285, and two light chains comprise a light chain variable region consisting of the amino acid sequence set forth in SEQ ID NO: 286 are excluded.
[4] The bispecific antibody according to any of [1] to [3], wherein the first antigen-binding polypeptide binding to amyloid beta (Aβ) is a first antigen-binding polypeptide binding to amyloid beta (Aβ) protofibril.
[5] The bispecific antibody according to any of [1] to [4], wherein the second antigen-binding polypeptide comprises HCDR2 consisting of the amino acid sequence set forth in SEQ ID NO: 8.
[6] The bispecific antibody according to [5], wherein the second antigen-binding polypeptide comprises a heavy chain variable region consisting of the amino acid sequence set forth in SEQ ID NO: 66 and a light chain variable region consisting of the amino acid sequence set forth in SEQ ID NO: 65.
[7] The bispecific antibody according to [5], wherein the second antigen-binding polypeptide comprises a heavy chain variable region consisting of the amino acid sequence set forth in SEQ ID NO: 267 and a light chain variable region consisting of the amino acid sequence set forth in SEQ ID NO: 265.
[8] The bispecific antibody according to any of [1] to [7], wherein the bispecific antibody binds to an amino acid residue corresponding to at least one amino acid residue selected from the group consisting of D245, Y247, P249, E350, G351, D352, C353, P354, S355, S361, M365, E369, and Q721 of a human transferrin receptor (TfR) consisting of the amino acid sequence set forth in SEQ ID NO: 256.
[9] The bispecific antibody according to [8], wherein D245, Y247, P249, E350, G351, D352, C353, P354, S355, S361, M365, E369, and Q721 of the human transferrin receptor (TfR) consisting of the amino acid sequence set forth in SEQ ID NO: 256 are D245, D247, P249, E350, G351, D352, C353, P354, S355, S361, M365, E369, and Q721 of a recombinant monkey transferrin receptor (cmTfR) protein consisting of the amino acid sequence set forth in SEQ ID NO: 257, respectively.
[10] The bispecific antibody according to [1] to [7], wherein the bispecific antibody binds to at least one amino acid residue selected from the group consisting of D352, S355, and S361 of the human transferrin receptor (TfR) consisting of the amino acid sequence set forth in SEQ ID NO: 256.
[11] The bispecific antibody according to [10], wherein the bispecific antibody binds to amino acid residues of D352, S355, and S361 of the human transferrin receptor (TfR) consisting of the amino acid sequence set forth in SEQ ID NO: 256.
[12] The bispecific antibody according to [10] or [11], wherein D352, S355, and S361 of the human transferrin receptor (TfR) consisting of the amino acid sequence set forth in SEQ ID NO: 256 are D352, S355, and S361 of a recombinant monkey transferrin receptor (cmTfR) protein consisting of the amino acid sequence set forth in SEQ ID NO: 257, respectively.
[13] The bispecific antibody according to any of [1] to [4], wherein the second antigen-binding polypeptide comprises HCDR2 consisting of the amino acid sequence set forth in SEQ ID NO: 13.
[14] The bispecific antibody according to [13], wherein the second antigen-binding polypeptide comprises a heavy chain variable region consisting of the amino acid sequence set forth in SEQ ID NO: 72 and a light chain variable region consisting of the amino acid sequence set forth in SEQ ID NO: 65.
[15] The bispecific antibody according to [13], wherein the second antigen-binding polypeptide comprises a heavy chain variable region consisting of the amino acid sequence set forth in SEQ ID NO: 275 and a light chain variable region consisting of the amino acid sequence set forth in SEQ ID NO: 273.
[16] The bispecific antibody according to any of [1] to [15], wherein a constant region of the first heavy chain and a constant region of the second heavy chain are human IgG1 heavy chain constant regions.
[17] The bispecific antibody according to [16], wherein the constant region of the first heavy chain and the constant region of the second heavy chain comprise a mutation wherein an asparagine residue at position 297 according to EU numbering has been substituted with a glycine residue (N297G) in the human IgG1 heavy chain constant region.
[18] The bispecific antibody according to [16] or [17], wherein the constant region of the first heavy chain comprises a mutation wherein a lysine residue at position 409 has been substituted with an aspartate residue (K409D) and a mutation wherein a lysine residue at position 439 has been substituted with a glutamic acid residue (K439E) according to EU numbering, in the human IgG1 heavy chain constant region, and
   the constant region of the second heavy chain comprises a mutation wherein an aspartate residue at position 356 has been substituted with a lysine residue (D356K) and a mutation wherein an aspartate residue at position 399 has been substituted with a lysine residue (D399K) according to EU numbering, in the human IgG1 heavy chain constant region.
[19] The bispecific antibody according to any of [16] to [18], wherein the lysine residue at the C-terminus of the first heavy chain is deleted.
[20] The bispecific antibody according to any of [16] to [19], wherein the constant region of the first heavy chain comprises N297G, K409D, and K439E, and
   the constant region of the second heavy chain comprises N297G, D356K, and D399K.
[21] The bispecific antibody according to any of [16] to [20], wherein the constant region of the first heavy chain and the constant region of the second heavy chain comprise N297G, and
   the lysine residue at the C-terminus of the first heavy chain is deleted.
[22] The bispecific antibody according to any of [16] to [21], wherein the constant region of the first heavy chain comprises K409D and K439E,
   the constant region of the second heavy chain comprises D356K and D399K, and
   the lysine residue at the C-terminus of the first heavy chain is deleted.
[23] The bispecific antibody according to any of [16] to [22], wherein the constant region of the first heavy chain comprises N297G, K409D, and K439E,
   the constant region of the second heavy chain comprises N297G, D356K, and D399K, and
   the lysine residue at the C-terminus of the first heavy chain is deleted.
[24] The bispecific antibody according to any of [16] to [23], wherein the constant region of the first heavy chain comprises the amino acid sequence set forth in SEQ ID NO: 240.
[25] The bispecific antibody according to any of [16] to [24], wherein the constant region of the second heavy chain comprises the amino acid sequence set forth in SEQ ID NO: 241.
[26] The bispecific antibody according to any of [16] to [25], wherein the constant region of the first heavy chain comprises the amino acid sequence set forth in SEQ ID NO: 240, and
   the constant region of the second heavy chain comprises the amino acid sequence set forth in SEQ ID NO: 241.
[27] The bispecific antibody according to any of [16] to [26], wherein a light chain constant region of the first antigen-binding polypeptide comprises the amino acid sequence set forth in SEQ ID NO: 119.
[28] The bispecific antibody according to any of [1] to [27], wherein the second antigen-binding polypeptide comprises scFv (single-chain Fv) or dsscFv (disulfide-stabilized single-chain Fv).
[29] The bispecific antibody according to [28], wherein the second antigen-binding polypeptide comprises scFv.
[30] The bispecific antibody according to [29], wherein the scFv consists of the amino acid sequence set forth in SEQ ID NO: 33.
[31] The bispecific antibody according to [29], wherein the scFv consists of the amino acid sequence set forth in SEQ ID NO: 45.
[32] The bispecific antibody according to [29], wherein the scFv consists of the amino acid sequence set forth in SEQ ID NO: 289.
[33] The bispecific antibody according to [29], wherein the scFv consists of the amino acid sequence set forth in SEQ ID NO: 290.
[34] The bispecific antibody according to [28], wherein the second antigen-binding polypeptide comprises dsscFv.
[35] The bispecific antibody according to [34], wherein the dsscFv consists of the amino acid sequence set forth in SEQ ID NO: 263.
[36] The bispecific antibody according to [34], wherein the dsscFv consists of the amino acid sequence set forth in SEQ ID NO: 271.
[37] The bispecific antibody according to [34], wherein the dsscFv consists of the amino acid sequence set forth in SEQ ID NO: 291.
[38] The bispecific antibody according to [34], wherein the dsscFv consists of the amino acid sequence set forth in SEQ ID NO: 292.
[39] The bispecific antibody according to any of [1] to [38], wherein the second antigen-binding polypeptide is connected to the C-terminus of the constant region of the second heavy chain of the first antigen-binding polypeptide via a linker.
[40] The bispecific antibody according to [39], wherein the linker is a peptide linker.
[41] The bispecific antibody according to [40], wherein the peptide linker is a peptide linker consisting of the amino acid sequence set forth in SEQ ID NO: 79.
[42A] A bispecific antibody binding to amyloid beta (Aβ) and a human transferrin receptor (TfR), wherein
   the bispecific antibody comprises a polypeptide consisting of the amino acid sequence set forth in SEQ ID NO: 189, a polypeptide consisting of the amino acid sequence set forth in SEQ ID NO: 191, and a polypeptide consisting of the amino acid sequence set forth in SEQ ID NO: 197.
[42B] The bispecific antibody according to [42A], wherein
   the bispecific antibody comprises two polypeptides consisting of the amino acid sequence set forth in SEQ ID NO: 197.
[42C] The bispecific antibody according to [42A], wherein
   the bispecific antibody comprises a first polypeptide consisting of the amino acid sequence set forth in SEQ ID NO: 189, a second polypeptide consisting of the amino acid sequence set forth in SEQ ID NO: 191, and a third polypeptide consisting of the amino acid sequence set forth in SEQ ID NO: 197.
[42D] The bispecific antibody according to [42C], wherein
   the first polypeptide comprises a first heavy chain,
   the second polypeptide comprises a second heavy chain, a peptide linker and a scFv, and
   the third polypeptide comprises a light chain.
[42E] The bispecific antibody according to [42C] or [42D], wherein
   the bispecific antibody comprises two third polypeptides.
[42F] The bispecific antibody according to [42E], wherein
   the first heavy chain is connected to one of the light chains, and
   the second heavy chain is connected to the other of the light chains.
[42G] The bispecific antibody according to any of [42D] to [42F], wherein
   the scFv is connected to the second heavy chain via the peptide linker.
[42H] The bispecific antibody according to [42G], wherein
   the scFv is connected to a C-terminus of the constant region of the second heavy chain via the peptide linker.
[43A] A bispecific antibody binding to amyloid beta (Aβ) and a human transferrin receptor (TfR), wherein
   the bispecific antibody comprises a polypeptide consisting of the amino acid sequence set forth in SEQ ID NO: 189, a polypeptide consisting of the amino acid sequence set forth in SEQ ID NO: 193, and a polypeptide consisting of the amino acid sequence set forth in SEQ ID NO: 197.
[43B] The bispecific antibody according to [43A], wherein
   the bispecific antibody comprises two polypeptides consisting of the amino acid sequence set forth in SEQ ID NO: 197.
[43C] The bispecific antibody according to [43A], wherein
   the bispecific antibody comprises a first polypeptide consisting of the amino acid sequence set forth in SEQ ID NO: 189, a second polypeptide consisting of the amino acid sequence set forth in SEQ ID NO: 193, and a third polypeptide consisting of the amino acid sequence set forth in SEQ ID NO: 197.
[43D] The bispecific antibody according to [43C], wherein
   the first polypeptide comprises a first heavy chain,
   the second polypeptide comprises a second heavy chain, a peptide linker and a scFv, and
   the third polypeptide comprises a light chain.
[43E] The bispecific antibody according to [43C] or [43D], wherein
   the bispecific antibody comprises two third polypeptides.
[43F] The bispecific antibody according to [43E], wherein
   the first heavy chain is connected to one of the light chains, and
   the second heavy chain is connected to the other of the light chains.
[43G] The bispecific antibody according to any of [43D] to [43F], wherein
   the scFv is connected to the second heavy chain via the peptide linker.
[43H] The bispecific antibody according to [43G], wherein
   the scFv is connected to a C-terminus of the constant region of the second heavy chain via the peptide linker.
[44A] A bispecific antibody binding to amyloid beta (Aβ) and a human transferrin receptor (TfR), wherein
   the bispecific antibody comprises a polypeptide consisting of the amino acid sequence set forth in SEQ ID NO: 189, a polypeptide consisting of the amino acid sequence set forth in SEQ ID NO: 269, and a polypeptide consisting of the amino acid sequence set forth in SEQ ID NO: 197.
[44B] The bispecific antibody according to [44A], wherein
   the bispecific antibody comprises two polypeptides consisting of the amino acid sequence set forth in SEQ ID NO: 197.
[44C] The bispecific antibody according to [44A], wherein
   the bispecific antibody comprises a first polypeptide consisting of the amino acid sequence set forth in SEQ ID NO: 189, a second polypeptide consisting of the amino acid sequence set forth in SEQ ID NO: 269, and a third polypeptide consisting of the amino acid sequence set forth in SEQ ID NO: 197.
[44D] The bispecific antibody according to [44C], wherein
   the first polypeptide comprises a first heavy chain,
   the second polypeptide comprises a second heavy chain, a peptide linker and a dsscFv, and
   the third polypeptide comprises a light chain.
[44E] The bispecific antibody according to [44C] or [44D], wherein
   the bispecific antibody comprises two third polypeptides.
[44F] The bispecific antibody according to [44E], wherein
   the first heavy chain is connected to one of the light chains, and
   the second heavy chain is connected to the other of the light chains.
[44G] The bispecific antibody according to any of [44D] to [44F], wherein
   the dsscFv is connected to the second heavy chain via the peptide linker.
[44H] The bispecific antibody according to [44G], wherein
   the dsscFv is connected to a C-terminus of the constant region of the second heavy chain via the peptide linker.
[45A] A bispecific antibody binding to amyloid beta (Aβ) and a human transferrin receptor (TfR), wherein
   the bispecific antibody comprises a polypeptide consisting of the amino acid sequence set forth in SEQ ID NO: 189, a polypeptide consisting of the amino acid sequence set forth in SEQ ID NO: 277, and a polypeptide consisting of the amino acid sequence set forth in SEQ ID NO: 197.
[45B] The bispecific antibody according to [45A], wherein
   the bispecific antibody comprises two polypeptides consisting of the amino acid sequence set forth in SEQ ID NO: 197.
[45C] The bispecific antibody according to [45A], wherein
   the bispecific antibody comprises a first polypeptide consisting of the amino acid sequence set forth in SEQ ID NO: 189, a second polypeptide consisting of the amino acid sequence set forth in SEQ ID NO: 277, and a third polypeptide consisting of the amino acid sequence set forth in SEQ ID NO: 197.
[45D] The bispecific antibody according to [45C], wherein
   the first polypeptide comprises a first heavy chain,
   the second polypeptide comprises a second heavy chain, a peptide linker and a dsscFv, and
   the third polypeptide comprises a light chain.
[45E] The bispecific antibody according to [45C] or [45D], wherein
   the bispecific antibody comprises two third polypeptides.
[45F] The bispecific antibody according to [45E], wherein
   the first heavy chain is connected to one of the light chains, and
   the second heavy chain is connected to the other of the light chains.
[45G] The bispecific antibody according to any of [45D] to [45F], wherein
   the dsscFv is connected to the second heavy chain via the peptide linker.
[45H] The bispecific antibody according to [45G], wherein
   the dsscFv is connected to a C-terminus of the constant region of the second heavy chain via the peptide linker.
[46] A bispecific antibody binding to amyloid beta (Aβ) and a human transferrin receptor (TfR), wherein
   the bispecific antibody comprises a polypeptide consisting of the amino acid sequence set forth in SEQ ID NO: 189, a polypeptide consisting of the amino acid sequence set forth in SEQ ID NO: 296, and a polypeptide consisting of the amino acid sequence set forth in SEQ ID NO: 197.
[47] A bispecific antibody binding to amyloid beta (Aβ) and a human transferrin receptor (TfR), wherein
   the bispecific antibody comprises a polypeptide consisting of the amino acid sequence set forth in SEQ ID NO: 189, a polypeptide consisting of the amino acid sequence set forth in SEQ ID NO: 298, and a polypeptide consisting of the amino acid sequence set forth in SEQ ID NO: 197.
[48] A bispecific antibody binding to amyloid beta (Aβ) and a human transferrin receptor (TfR), wherein
   the bispecific antibody comprises a polypeptide consisting of the amino acid sequence set forth in SEQ ID NO: 189, a polypeptide consisting of the amino acid sequence set forth in SEQ ID NO: 300, and a polypeptide consisting of the amino acid sequence set forth in SEQ ID NO: 197.
[49] A bispecific antibody binding to amyloid beta (Aβ) and a human transferrin receptor (TfR), wherein
   the bispecific antibody comprises a polypeptide consisting of the amino acid sequence set forth in SEQ ID NO: 189, a polypeptide consisting of the amino acid sequence set forth in SEQ ID NO: 302, and a polypeptide consisting of the amino acid sequence set forth in SEQ ID NO: 197.
[50] The bispecific antibody according to any of [42] to [49], wherein the bispecific antibody binding to amyloid beta (Aβ) and a human transferrin receptor (TfR) is a bispecific antibody binding to amyloid beta (Aβ) protofibril and a human transferrin receptor (TfR).
[51] A bispecific antibody comprising a first antigen-binding polypeptide binding to amyloid beta (Aβ) and a second antigen-binding polypeptide binding to a human transferrin receptor (TfR), wherein:
(i) the first antigen-binding polypeptide comprises a first heavy chain consisting of the amino acid sequence set forth in SEQ ID NO: 238 and the amino acid sequence set forth in SEQ ID NO: 240, a second heavy chain consisting of the amino acid sequence set forth in SEQ ID NO: 238 and the amino acid sequence set forth in SEQ ID NO: 241, and a light chain consisting of the amino acid sequence set forth in SEQ ID NO: 239 and the amino acid sequence set forth in SEQ ID NO: 119;
(ii) the second antigen-binding polypeptide comprises scFv consisting of the amino acid sequence set forth in SEQ ID NO: 33; and
(iii) the second antigen-binding polypeptide is connected to the C-terminus of a constant region of the second heavy chain of the first antigen-binding polypeptide, via a peptide linker consisting of the amino acid sequence set forth in SEQ ID NO: 79.
[52] A bispecific antibody comprising a first antigen-binding polypeptide binding to amyloid beta (Aβ) and a second antigen-binding polypeptide binding to a human transferrin receptor (TfR), wherein:
(i) the first antigen-binding polypeptide comprises a first heavy chain consisting of the amino acid sequence set forth in SEQ ID NO: 238 and the amino acid sequence set forth in SEQ ID NO: 240, a second heavy chain consisting of the amino acid sequence set forth in SEQ ID NO: 238 and the amino acid sequence set forth in SEQ ID NO: 241, and a light chain consisting of the amino acid sequence set forth in SEQ ID NO: 239 and the amino acid sequence set forth in SEQ ID NO: 119;
(ii) the second antigen-binding polypeptide comprises scFv consisting of the amino acid sequence set forth in SEQ ID NO: 45; and
(iii) the second antigen-binding polypeptide is connected to the C-terminus of a constant region of the second heavy chain of the first antigen-binding polypeptide, via a peptide linker consisting of the amino acid sequence set forth in SEQ ID NO: 79.
[53] A bispecific antibody comprising a first antigen-binding polypeptide binding to amyloid beta (Aβ) and a second antigen-binding polypeptide binding to a human transferrin receptor (TfR), wherein:
(i) the first antigen-binding polypeptide comprises a first heavy chain consisting of the amino acid sequence set forth in SEQ ID NO: 238 and the amino acid sequence set forth in SEQ ID NO: 240, a second heavy chain consisting of the amino acid sequence set forth in SEQ ID NO: 238 and the amino acid sequence set forth in SEQ ID NO: 241, and a light chain consisting of the amino acid sequence set forth in SEQ ID NO: 239 and the amino acid sequence set forth in SEQ ID NO: 119;
(ii) the second antigen-binding polypeptide comprises dsscFv consisting of the amino acid sequence set forth in SEQ ID NO: 263; and
(iii) the second antigen-binding polypeptide is connected to the C-terminus of a constant region of the second heavy chain of the first antigen-binding polypeptide, via a peptide linker consisting of the amino acid sequence set forth in SEQ ID NO: 79.
[54] A bispecific antibody comprising a first antigen-binding polypeptide binding to amyloid beta (Aβ) and a second antigen-binding polypeptide binding to a human transferrin receptor (TfR), wherein:
(i) the first antigen-binding polypeptide comprises a first heavy chain consisting of the amino acid sequence set forth in SEQ ID NO: 238 and the amino acid sequence set forth in SEQ ID NO: 240, a second heavy chain consisting of the amino acid sequence set forth in SEQ ID NO: 238 and the amino acid sequence set forth in SEQ ID NO: 241, and a light chain consisting of the amino acid sequence set forth in SEQ ID NO: 239 and the amino acid sequence set forth in SEQ ID NO: 119;
(ii) the second antigen-binding polypeptide comprises dsscFv consisting of the amino acid sequence set forth in SEQ ID NO: 271; and
(iii) the second antigen-binding polypeptide is connected to the C-terminus of a constant region of the second heavy chain of the first antigen-binding polypeptide, via a peptide linker consisting of the amino acid sequence set forth in SEQ ID NO: 79.
[55] A bispecific antibody comprising a first antigen-binding polypeptide binding to amyloid beta (Aβ) and a second antigen-binding polypeptide binding to a human transferrin receptor (TfR), wherein:
(i) the first antigen-binding polypeptide comprises a first heavy chain consisting of the amino acid sequence set forth in SEQ ID NO: 238 and the amino acid sequence set forth in SEQ ID NO: 240, a second heavy chain consisting of the amino acid sequence set forth in SEQ ID NO: 238 and the amino acid sequence set forth in SEQ ID NO: 241, and a light chain consisting of the amino acid sequence set forth in SEQ ID NO: 239 and the amino acid sequence set forth in SEQ ID NO: 119;
(ii) the second antigen-binding polypeptide comprises scFv consisting of the amino acid sequence set forth in SEQ ID NO: 289; and
(iii) the second antigen-binding polypeptide is connected to the C-terminus of a constant region of the second heavy chain of the first antigen-binding polypeptide, via a peptide linker consisting of the amino acid sequence set forth in SEQ ID NO: 79.
[56] A bispecific antibody comprising a first antigen-binding polypeptide binding to amyloid beta (Aβ) and a second antigen-binding polypeptide binding to a human transferrin receptor (TfR), wherein:
(i) the first antigen-binding polypeptide comprises a first heavy chain consisting of the amino acid sequence set forth in SEQ ID NO: 238 and the amino acid sequence set forth in SEQ ID NO: 240, a second heavy chain consisting of the amino acid sequence set forth in SEQ ID NO: 238 and the amino acid sequence set forth in SEQ ID NO: 241, and a light chain consisting of the amino acid sequence set forth in SEQ ID NO: 239 and the amino acid sequence set forth in SEQ ID NO: 119;
(ii) the second antigen-binding polypeptide comprises scFv consisting of the amino acid sequence set forth in SEQ ID NO: 290; and
(iii) the second antigen-binding polypeptide is connected to the C-terminus of a constant region of the second heavy chain of the first antigen-binding polypeptide, via a peptide linker consisting of the amino acid sequence set forth in SEQ ID NO: 79.
[57] A bispecific antibody comprising a first antigen-binding polypeptide binding to amyloid beta (Aβ) and a second antigen-binding polypeptide binding to a human transferrin receptor (TfR), wherein:
(i) the first antigen-binding polypeptide comprises a first heavy chain consisting of the amino acid sequence set forth in SEQ ID NO: 238 and the amino acid sequence set forth in SEQ ID NO: 240, a second heavy chain consisting of the amino acid sequence set forth in SEQ ID NO: 238 and the amino acid sequence set forth in SEQ ID NO: 241, and a light chain consisting of the amino acid sequence set forth in SEQ ID NO: 239 and the amino acid sequence set forth in SEQ ID NO: 119;
(ii) the second antigen-binding polypeptide comprises dsscFv consisting of the amino acid sequence set forth in SEQ ID NO: 291; and
(iii) the second antigen-binding polypeptide is connected to the C-terminus of a constant region of the second heavy chain of the first antigen-binding polypeptide, via a peptide linker consisting of the amino acid sequence set forth in SEQ ID NO: 79.
[58] A bispecific antibody comprising a first antigen-binding polypeptide binding to amyloid beta (Aβ) and a second antigen-binding polypeptide binding to a human transferrin receptor (TfR), wherein:
(i) the first antigen-binding polypeptide comprises a first heavy chain consisting of the amino acid sequence set forth in SEQ ID NO: 238 and the amino acid sequence set forth in SEQ ID NO: 240, a second heavy chain consisting of the amino acid sequence set forth in SEQ ID NO: 238 and the amino acid sequence set forth in SEQ ID NO: 241, and a light chain consisting of the amino acid sequence set forth in SEQ ID NO: 239 and the amino acid sequence set forth in SEQ ID NO: 119;
(ii) the second antigen-binding polypeptide comprises dsscFv consisting of the amino acid sequence set forth in SEQ ID NO: 292; and
(iii) the second antigen-binding polypeptide is connected to the C-terminus of a constant region of the second heavy chain of the first antigen-binding polypeptide, via a peptide linker consisting of the amino acid sequence set forth in SEQ ID NO: 79.
[59] The bispecific antibody according to any of [51] to [58], wherein the first antigen-binding polypeptide binding to amyloid beta (Aβ) is a first antigen-binding polypeptide binding to amyloid beta (Aβ) protofibril.
[60] An isolated nucleic acid encoding the bispecific antibody according to any of [1] to [59].
[61] A vector comprising the nucleic acid according to [60].
[62] A host cell comprising the nucleic acid according to or the vector according to [61].
[63] A method for producing a bispecific antibody, comprising a step of culturing the host cell according to [62].
[64] A pharmaceutical composition, comprising the bispecific antibody according to any of [1] to [59].
[65] The pharmaceutical composition according to [64], comprising at least one pharmaceutically acceptable carrier.
[66] The pharmaceutical composition according to [64] or [65], for treatment of an amyloid beta (Aβ)-related disease.
[67] The bispecific antibody according to any of [1] to [59], for use in the treatment of an amyloid beta (Aβ)-related disease.
[68] A method of treating an amyloid beta (Aβ)-related disease, comprising administering an effective amount of the bispecific antibody according to any of [1] to [59] to a subject in need thereof.
[89] Use of the bispecific antibody according to any of [1] to [59], in the manufacture of a medicament for the treatment of an amyloid beta (Aβ)-related disease.
[70] Use of the bispecific antibody according to any of [1] to [59], for the treatment of an amyloid beta (Aβ)-related disease.
[71] The pharmaceutical composition, bispecific antibody, method of treating, or use according to any of [66] to [70], wherein the Aβ-related disease is Alzheimer's disease (AD), mild cognitive impairment due to AD (MCI due to AD), preclinical AD, or Down's syndrome.
[72] The pharmaceutical composition, bispecific antibody, method of treating, or use according to any of [66] to [70], wherein the Aβ-related disease is Alzheimer's disease (AD), mild cognitive impairment due to AD (MCI due to AD), or preclinical AD.
[73] The pharmaceutical composition, bispecific antibody, method of treating, or use according to any of [66] to [72], wherein the Aβ-related disease is Alzheimer's disease (AD).
[74] The pharmaceutical composition, bispecific antibody, method of treating, or use according to any of [66] to [72], wherein the Aβ-related disease is mild cognitive impairment due to AD (MCI due to AD).
[75] The pharmaceutical composition, bispecific antibody, method of treating, or use according to any of [66] to [72], wherein the Aβ-related disease is preclinical AD.
[76] The pharmaceutical composition, bispecific antibody, method of treating, or use according to any of [66] to [72], wherein the Aβ-related disease is Down's syndrome.

### Advantageous Effects of Invention

According to the bispecific antibody of the present disclosure, an anti-amyloid β (Aβ) antibody or an antigen-binding fragment thereof can be transferred into the brain.

### BRIEF DESCRIPTION OF THE DRAWINGS

FIG. 1 shows the results of evaluating the pH-dependent binding of the bispecific anti-human TfR/EphA4 antibody (#12-aT04mv) at various concentrations to human TfR in Experimental Example 2, as determined by absorbance.

FIG. 2 shows the binding reaction curves of #12-aT04mv at various dilution concentrations to human TfR in Experimental Example 3.

FIG. 3 shows the results of flow cytometry analysis using B300.19 cells producing a human/mouse TfR chimera (h/m TfR 184-385) in Experimental Example 4. Panel (a) shows the results for control B300.19 cells, and panel (b) shows the results for B300.19 cells producing the h/m TfR 184-385 chimera.

FIG. 4 shows the genetic modification for generating a human TfR knock-in mouse (hTfR-KI mouse) in Experimental Example 5.

FIG. 5 shows the concentrations of #12 and #12-aT04mv in plasma (a) and in hippocampal tissue lysate (b) in mice to which #12 and #12-aT04mv have been administered in Experimental Example 5.

FIG. 6 shows images of the intracerebral distribution of #12 and #12-aT04mv in mice to which #12 and #12-aT04mv have been administered in Experimental Example 5.

FIG. 7A shows the results of evaluating the pH-dependent binding of bispecific anti-human TfR/EphA4 mutant antibodies to human and monkey TfR in Experimental Example 7. Panel (a) shows the results for #12-aT04m02mv, panel (b) shows the results for #12-aT04m0201mv, panel (c) shows the results for #12-aT04m0202mv, and panel (d) shows the results for #12-aT04m0203mv, respectively.

FIG. 7B shows the results of evaluating the pH-dependent binding of bispecific anti-human TfR/EphA4 mutant antibodies to human and monkey TfR in Experimental Example 7. Panel (a) shows the results for #12-aT04m0204mv, panel (b) shows the results for #12-aT04m0205mv, panel (c) shows the results for #12-aT04m0206mv, and panel (d) shows the results for #12-aT04m0207mv, respectively.

FIG. 8 shows the concentrations of bispecific anti-human TfR/EphA4 mutant antibodies in plasma (a) and in hippocampal tissue lysate (b) in mice to which the bispecific anti-human TfR/EphA4 mutant antibodies have been administered in Experimental Example 9.

FIG. 9 shows images of the intracerebral distribution of bispecific anti-human TfR/EphA4 mutant antibodies in mice to which the bispecific anti-human TfR/EphA4 mutant antibodies have been administered in Experimental Example 9.

FIG. 10 shows the binding response curves and the KD values of the monoclonal antibodies BR260 (aT04-Fab-His6) and BR261 (aT04m02-Fab-His6) to the His-human TfR protein in Experimental Example 10. Panel (a) shows the binding response curve of aT04-Fab-His6, panel (b) shows the binding response curve of aT04m02-Fab-His6, and panel (c) shows various kinetics parameters, respectively.

FIG. 11A shows (a) the crystal structure analysis of the complex of aT04-Fab-his6 and the extracellular region of human transferrin receptor (hTfR), and (b) the Cryo-electron microscopy analysis of the complex of aT04m02-Fab-his6, the extracellular region of human transferrin receptor (hTfR), and holo-type human transferrin (holo Tf) in Experimental Example 11.

FIG. 11B shows the recognition sites on hTfR that aT04-Fab recognizes in Experimental Example 11. Portions highlighted in grey indicate the amino acid residues of the recognition sites, and the boxed sequences indicate the apical domain.

FIG. 11C shows recognition sites on hTfR that aT04m02-Fab recognizes in Experimental Example 11. Portions highlighted in grey indicate the amino acid residues of the recognition sites, and the boxed sequences indicate the apical domain.

FIG. 11D shows the structure of the recognition sites of aT04m02-Fab-his6 that recognize human TfR in Experimental Example 11.

FIG. 12 shows schematic diagrams of each bispecific anti-human TfR/AB antibody used in Experimental Example 12.

FIG. 13A shows the results of evaluating the binding activity of (a) BR11 and BR282, and (b) BR15, BR118, and BR24, to human TfR by ELISA in Experimental Example 13.

FIG. 13B shows the results of evaluating the binding activity of (a) BR11 and BR282 and (b) BR15, BR118, and BR24, to protofibril-like Aβ by ELISA in Experimental Example 13.

FIG. 14A shows the results of measuring the antibody concentrations in plasma when (a) BR11 and (b) BR282 were administered to APP KI/hTfR-KI (homo/homo) mice in Experimental Example 14.

FIG. 14B shows the results of quantitative analysis of amyloid plaque areas when BR11 and BR282 were administered to APP KI/hTfR-KI (homo/homo) mice in Experimental Example 14.

FIG. 14C shows the results of biochemical quantification of oligomer Aβ and insoluble Aβ in brain tissue when BR11 and BR282 were administered to APP KI/hTfR-KI (homo/homo) mice in Experimental Example 14. FIG. 14C shows the results of (a) oligomer Aβ in the supernatant obtained by centrifugation of homogenate of right brain tissue at 16,000×g (16k_sup), (b) insoluble Aβ in the precipitation obtained by centrifugation of the homogenate of right brain tissue at 16,000×g (16k_ppt), (c) oligomer Aβ in the supernatant obtained by further ultracentrifugation at 100,000×g (16k/100k_sup), and (d) insoluble Aβ in the precipitation obtained by ultracentrifugation at 100,000×g (16k/100k_ppt), respectively.

FIG. 14D shows the results of quantifying p-tau in brain sections when BR11 and BR282 were administered to APP KI/hTfR-KI (homo/homo) mice in Experimental Example 14, showing the quantification results of phosphorylated tau231 (pTau231) area.

FIG. 15A shows the results of measuring the antibody concentrations in plasma when BR11 and BR282 were administered to 5xFAD/hTfR-KI (hemi/homo) mice in Experimental Example 15.

FIG. 15B shows the results of measuring the amount of oligomer Aβ in brain tissue when BR11 and BR282 were administered to 5xFAD/hTfR-KI (hemi/homo) mice in Experimental Example 15.

FIG. 15C shows the results of evaluating the ARIA risks by immune cell infiltration when BR11 and BR282 were administered to 5xFAD/hTfR-KI (hemi/homo) mice in Experimental Example 15.

FIG. 16A shows genetic modification to generate a human TfR-ApD knock-in mouse (hTfR-ApD-KI mouse) in Experimental Example 16.

FIG. 16B shows the results of reticulocyte (RET) of the hematology test for respective antibodies (BR15, BR118, and BR24) in Experimental Example 16.

FIG. 17 shows schematic diagrams of respective antibodies (BR188, BR218, BR189, and BR196) in Experimental Example 17.

FIG. 18A shows the results of evaluating the binding of BR188, BR218, BR189, and BR196 to human TfR by ELISA in Experimental Example 18.

FIG. 18B shows the results of evaluating the binding of BR188, BR218, BR189, and BR196 to protofibril-like Aβ by ELISA in Experimental Example 18.

FIG. 18C shows the results of measuring the binding affinity of BR188, BR218, BR189, and BR196 to monomer Aβ, protofibril, and human TfR by surface plasmon resonance in Experimental Example 18.

FIG. 18D shows the results of flow cytometry analysis using U2OS cells for BR188, BR218, BR189, and BR196 in Experimental Example 18.

FIG. 19A shows the results of evaluating the binding affinity of BR188, BR218, BR189, and BR196 to human FcγRIIIA, human FcγRIIA, human FcγRI, and human FcRn by surface plasmon resonance in Experimental Example 19.

FIG. 19B shows the results of the antibody-dependent cellular cytotoxicity (ADCC) assay for BR188, BR218, BR189, and BR196 in Experimental Example 19.

FIG. 19C shows the results of evaluating the binding properties of BR188, BR218, BR189, and BR196 to human C1q by ELISA in Experimental Example 19.

FIG. 19D shows the results of ex vivo amyloid plaque phagocytosis of BR188, BR218, BR189, and BR196 in Experimental Example 19.

FIG. 20 shows the antibody concentrations when respective antibodies were administered to hTfR knock-in (hTfR-KI) (homo) mice in Experimental Example 20. Panels (a), (b), and (c) show the results of measuring the antibody concentrations in plasma, brain tissue, and cerebrospinal fluid (CSF), respectively.

FIG. 21 shows schematic diagrams of the respective antibodies (BR258, BR259, BR350, BR355, BR360, BR361, BR362, BR363, BR364, BR357, BR368, BR356, BR359, BR358, BR380, and BR397) in Experimental Example 21.

FIG. 22A shows schematic diagrams of the respective antibodies (BR259, BR355, BR357, BR356, BR359, BR358, BR360, BR361, BR362, BR363, and BR364) in Experimental Example 22.

FIG. 22B shows the SDS-PAGE results of purified BR259, BR355, BR357, BR356, BR359, BR358, BR360, BR361, BR362, BR363, and BR364 in Experimental Example 22. Panel (a) shows the results of non-reduced SDS-PAGE, panel (b) shows the results of reduced SDS-PAGE, and panel (c) shows the lanes and names of the respective antibodies, respectively.

FIG. 23 shows the results of LC-MS analysis of (a) BR259 and (b) BR357 in Experimental Example 23.

FIG. 24 shows the results (binding response curves and KD values) of evaluating the binding of BR259, BR357, BR350, and BR368 to human TfR (hTfR) and monkey TfR (cmTfR) in Experimental Example 24.

FIG. 25A shows the antibody productivity (bulk-pools titer) by stable cell lines of BR380 and BR397 in Experimental Example 25.

FIG. 25B shows the results of SDS-PAGE of antibody production by stable cell lines of BR380 and BR397 in Experimental Example 25.

FIG. 26 shows schematic diagrams of respective antibodies (BR220, BR218, BR258, BR292, BR259, BR317, BR383, BR384, BR387, BR388, BR380, BR381, and BR322) in Experimental Example 26.

FIG. 27A shows the results of evaluating the binding of BR220, BR218, BR258, BR259, BR380, BR383, and BR387 to human TfR in Experimental Example 27.

FIG. 27B shows the results of evaluating the binding of BR220, BR218, BR258, BR259, BR380, BR383, and BR387 to protofibril-like Aβ in Experimental Example 27.

FIG. 28 shows the antibody concentrations when respective antibodies were administered to hTfR-ApD-KI (homo) mice in Experimental Example 28. Panels (a), (b), and (c) show the results of measuring the antibody concentrations in plasma, brain tissue, and CSF, respectively.

FIG. 29A shows the antibody productivity of respective antibodies (BR258, BR317, BR383, BR259, BR322, and BR380) produced by stable cell lines in Experimental Example 29.

FIG. 29B shows the antibody productivity of respective antibodies (BR383, BR384, BR387, BR388, BR380, and BR381) produced by stable cell lines in Experimental Example 29.

FIG. 29C shows the antibody productivity of respective antibodies (BR258, BR292, BR383, BR387, BR259, BR350, and BR380) produced by stable cell lines in Experimental Example 29.

FIG. 29D shows the results of SDS-PAGE analysis of purified BR292, BR317, BR382, BR383, BR384, BR387, and BR388) in Experimental Example 29. Panel (a) shows the results of non-reduced SDS-PAGE, and panel (b) shows the results of reduced SDS-PAGE, respectively.

FIG. 30A shows the results of separating BR259 by HPLC cation exchange chromatography and analyzing each fraction by SDS-PAGE in Experimental Example 30. Panel (a) shows the results of HPLC cation exchange chromatography, and panel (b) shows the results of SDS-PAGE (left: non-reduced; and right: reduced), respectively.

FIG. 30B shows the summary of results of separating respective antibodies (BR380, BR383, and BR292) by affinity chromatography and cation exchange chromatography, and analyzing each fraction by SDS-PAGE in Experimental Example 30.

FIG. 31A shows the results of binding evaluation of respective antibodies (BR380, BR381, BR383, BR384, BR387, and BR388) to human TfR in Experimental Example 31.

FIG. 31B shows the results of binding evaluation of respective antibodies (BR380, BR381, BR383, BR384, BR387, and BR388) to protofibril-like Aβ in Experimental Example 31.

FIG. 32A shows the results of binding evaluation of respective antibodies (BR380, BR381, BR383, BR384, BR387, and BR388) to human C1q in Experimental Example 32.

FIG. 32B shows the results of binding evaluation of respective antibodies (BR380, BR381, BR383, BR384, BR387, and BR388) to mouse C1q in Experimental Example 32.

FIG. 33A shows the results of evaluating the acid resistance of respective antibodies (BR292, BR384, and BR388) in Experimental Example 33. Panel (a) shows the percentage of soluble aggregates (%), and panel (b) shows the percentage of fragments (%).

FIG. 33B shows the results of separating BR383 by HPLC cation exchange chromatography and analyzing each fraction by SDS-PAGE in Experimental Example 33. Panel (a) shows the results of HPLC cation exchange chromatography, and panel (b) shows the results of SDS-PAGE, respectively.

FIG. 33C shows the results of separating BR292 by HPLC cation exchange chromatography and analyzing each fraction by SDS-PAGE in Experimental Example 33. Panel (a) shows the results of HPLC cation exchange chromatography, panel (b) shows the results of SDS-PAGE, and panel (c) shows the summary of results of each fraction from BR383 and BR292, respectively.

FIG. 34 shows schematic diagrams of respective antibodies (BR467, BR441, BR468, BR663, BR665, BR443, BR445, BR664, BR666, BR442, BR469, BR444, and BR446) in Experimental Example 34.

FIG. 35A shows the results of evaluating the binding affinity of respective antibodies (BR467, BR441, BR442, BR443, and BR444) to monomer Aβ and protofibril in Experimental Example 35.

FIG. 35B shows the results of evaluating the binding affinity of respective antibodies (BR467, BR468, BR469, BR445, and BR446) to monomer Aβ and protofibril in Experimental Example 35.

FIG. 35C shows the results of measuring the binding affinity (binding response curves and KD values) of respective antibodies (BR441, BR442, BR443, BR444, BR468, BR469, BR445, and BR446) to human TfR in Experimental Example 35.

FIG. 36A shows the antibody concentrations when respective antibodies (BR467, BR357, BR442, BR444, BR446, and BR469) were administered to hTfR-ApD-KI (homo) mice at a dose of 20 mg/kg, calculated as an IgG equivalent amount in Experimental Example 36. Panels (a), (b), and (c) show the results of measuring the antibody concentrations in plasma, brain tissue, and CSF, respectively.

FIG. 36B shows the antibody concentrations when respective antibodies (BR467, BR259, BR663, BR664, BR665, and BR666) were administered to hTfR-ApD-KI (homo) mice at a dose of 10 mg/kg, calculated as an IgG equivalent amount in Experimental Example 36. Panels (a), (b), and (c) show the results of measuring the antibody concentrations in plasma, brain tissue, and CSF, respectively.

FIG. 37A shows the results of measuring (a) reticulocyte and (b) iron when respective antibodies (BR467, BR357, BR442, BR444, BR446, and BR469) were administered to hTfR-ApD-KI mice at a dose of 20 mg/kg, calculated as an IgG equivalent amount in Experimental Example 37.

FIG. 37B shows the results of measuring (a) reticulocyte and (b) iron when respective antibodies (BR467, BR259, BR663, BR664, BR665, and BR666) were administered to hTfR-ApD-KI mice at a dose of 10 mg/kg, calculated as an IgG equivalent amount in Experimental Example 37.

FIG. 38 shows the antibody concentrations when antibodies BR357, BR446, and BR469 were administered to APP KI/hTfR-KI mice in Experimental Example 38.

FIG. 39A shows the antibody productivity (bulk-pools titer) of respective antibodies produced by stable cell lines in Experimental Example 39.

FIG. 39B shows the results of evaluating the purity (SDS-PAGE) of respective antibodies produced by stable cell lines in Experimental Example 39. Panel (a) shows the results of non-reduced SDS-PAGE, and panel (b) shows the results of reduced SDS-PAGE, respectively.

FIG. 40A shows schematic diagrams of respective antibodies (BR10, BR470, BR471, BR478, BR479, BR802, and BR803) in Experimental Example 40.

FIG. 40B shows the antibody productivity (bulk-pools titer) of respective antibodies produced by stable cell lines in Experimental Example 40.

FIG. 40C shows the results of evaluating the purity (non-reduced SDS-PAGE) of respective antibodies produced by stable cell lines in Experimental Example 40.

FIG. 40D shows the results of evaluating the purity (reduced SDS-PAGE) of respective antibodies produced by stable cell lines in Experimental Example 40.

FIG. 41A shows the results of separating respective antibodies (BR478 and BR479) by a Praesto Jetted A50 HipH (Purolite) column in Experimental Example 41.

FIG. 41B shows the results of separating respective antibodies (BR478 and BR479) by cation exchange chromatography in Experimental Example 41.

FIG. 42A shows the results of evaluating the binding of respective antibodies (BR478, BR479, and BR10) to human TfR in Experimental Example 42.

FIG. 42B shows the results of evaluating the binding of respective antibodies (BR478, BR479, and BR10) to protofibril-like Aβ in Experimental Example 42.

FIG. 42C shows the results (binding response curves and KD values) of evaluating the binding properties of respective antibodies (BR478, BR479, and BR10) to monomer Aβ and protofibril in Experimental Example 42.

FIG. 42D shows the results (binding response curves and KD values) of evaluating the binding of respective antibodies (BR478 and BR479) to human TfR and monkey TfR in Experimental Example 42.

FIG. 42E shows the results of flow cytometry using U2OS cells for respective antibodies (BR478, BR479, and BR10) in Experimental Example 42.

FIG. 43A shows the results of evaluating the binding affinity and KD values of BR10, BR478, and BR479 to human FcγRIIIA, human FcγRIIA, human FcγRI, and human FcRn by surface plasmon resonance in Experimental Example 43.

FIG. 43B shows the results of the antibody-dependent cellular cytotoxicity (ADCC) assay for BR10, BR478, and BR479 in Experimental Example 43.

FIG. 43C shows the results of evaluating the binding properties of BR10, BR478, and BR479 to human C1q in Experimental Example 43.

FIG. 43D shows the results of in vivo amyloid plaque phagocytosis of BR10, BR478, and BR479 in Experimental Example 43.

FIG. 44A shows the results of predicting immunogenicity of the variable regions of BR478 and BR479 in silico in Experimental Example 44.

FIG. 44B shows the results of predicting immunogenicity of the constant regions of BR478 and BR479 in silico in Experimental Example 44.

FIG. 45 shows the results of off-target screening for BR478 and BR479 in Experimental Example 45.

FIG. 46 shows the results of antibody concentrations when respective antibodies (BR10, BR478, and BR479) were administered to hTfR-ApD-KI (homo) mice at a dose of 10 mg/kg, calculated as an IgG equivalent amount in Experimental Example 46. Panels (a), (b), and (c) show the results of measuring the antibody concentrations in plasma, brain tissue, and CSF, respectively.

FIG. 47 shows the results of blood tests when respective antibodies (BR10, BR478, and BR479) were administered to hTfR-ApD-KI mice at a dose of 10 mg/kg, calculated as an IgG equivalent amount in Experimental Example 47. Panels (a) and (b) show the results of measuring reticulocyte and iron, respectively.

FIG. 48 shows the results of quantification of oligomer Aβ, Aβ protofibril in brain tissue when respective antibodies (BR10, BR478, and BR479) were administered to APP KI/hTfR-ApD-KI mice at a dose of 10 mg/kg or 30 mg/kg, calculated as an IgG equivalent amount in Experimental Example 48. Panels (a), (b) and (c) show the results of oligomer Aβ in 16k_sup, oligomer Aβ in 16k/100k_sup and Aβ protofibril in 16k_sup, respectively.

FIG. 49A shows the results of measuring the concentrations of respective antibodies in serum and CSF when BR478 and BR479 were administered to cynomolgus monkeys at a dose of 5 mg/kg and 30 mg/kg in Experimental Example 49. Panels (a), (b), (c), and (d) show the results of measuring the concentrations of the antibody BR478 in serum, the antibody BR479 in serum, the antibody BR478 in CSF, and the antibody BR479 in CSF, respectively.

FIG. 49B shows the results of measuring (a) reticulocyte and (b) iron in blood test when BR478 and BR479 were administered to cynomolgus monkeys at a dose of 5 and 30 mg/kg in Experimental Example 49.

FIG. 50A shows the distribution in the brain and the binding to amyloid plaque 3 days after the respective antibodies (BR10, BR478, and BR479) were administered to APP KI/hTfR-ApD-KI (homo/homo) mice at a dose of 10 mg/kg, calculated as an IgG equivalent amount in Experimental Example 50.

FIG. 50B shows the distribution in the brain and the binding to amyloid plaque 7 days after the respective antibodies (BR10, BR478, and BR479) were administered to APP KI/hTfR-ApD-KI (homo/homo) mice at a dose of 10 mg/kg, calculated as an IgG equivalent amount in Experimental Example 50.

FIG. 50C shows the distribution in the brain and the binding to amyloid plaque 14 days after the respective antibodies (BR10, BR478, and BR479) were administered to APP KI/hTfR-ApD-KI (homo/homo) mice at a dose of 10 mg/kg, calculated as an IgG equivalent amount in Experimental Example 50.

FIG. 51A shows the antibody productivity of respective antibodies (BR802 and BR803) produced by stable cell lines in Experimental Example 51.

FIG. 51B shows the results of evaluating the purity (non-reduced SDS-PAGE and reduced SDS-PAGE) of respective antibodies (BR802 and BR803) produced by stable cell lines in Experimental Example 51.

FIG. 52A shows the results of separating respective antibodies (BR802 and BR803) using a Praesto Jetted A50 HipH (Purolite) resin column in Experimental Example 52.

FIG. 52B shows the results of further separating respective antibodies (BR802 and BR803), previously separated using a Praesto Jetted A50 HipH (Purolite) resin column, by cation exchange chromatography using a SP-650M (TOSOH) resin column in Experimental Example 52.

FIG. 52C shows the results of evaluating the purity (non-reduced SDS-PAGE and reduced SDS-PAGE) of respective antibodies (BR802 and BR803) that separated by cation exchange chromatography using a resin column in Experimental Example 52.

FIG. 53A shows the results of evaluating the binding of respective antibodies (BR802, BR803 and BR10) to human TfR and monkey TfR by ELISA in Experimental Example 53.

FIG. 53B shows the results of evaluating the binding of respective antibodies (BR802, BR803 and BR10) to protofibril-like Aβ by ELISA in Experimental Example 53.

FIG. 53C shows the results of measuring the binding affinity of respective antibodies (BR802, BR803 and BR10) to monomer Aβ and protofibril by surface plasmon resonance (SPR method) in Experimental Example 53.

FIG. 53D shows the results of measuring the binding affinity of respective antibodies (BR802, BR803 and BR10) to human TfR and monkey TfR by surface plasmon resonance (SPR method) in Experimental Example 53.

FIG. 53E shows the results of flow cytometry analysis using U2OS cells for respective antibodies (BR802, BR803 and BR10) in Experimental Example 53.

FIG. 54A shows the results of evaluating the binding affinity of respective antibodies (BR802, BR803 and BR10) to human FcγRI, human FcγRIIA, human FcγRIIIA, and human FcRn by surface plasmon resonance (SPR method) in Experimental Example 54.

FIG. 54B shows the results of the antibody-dependent cellular cytotoxicity (ADCC) assay for BR10, BR802, and BR803 in Experimental Example 54.

FIG. 54C shows the results of evaluating the binding properties of BR10, BR802, and BR803 to human C1q in Experimental Example 54.

FIG. 54D shows the results of in vitro amyloid plaque phagocytosis in brain tissue of APP KI mice of BR10, BR802, and BR803 in Experimental Example 54.

FIG. 54E shows the results of in vitro amyloid plaque phagocytosis in brain tissue of Alzheimer's disease patients of BR10, BR802, and BR803 in Experimental Example 54.

FIG. 55 shows the antibody concentrations when respective antibodies (BR10, BR802, and BR803) were administered to hTfR-KI (hetero) mice at a dose of 10 mg/kg, calculated as an IgG equivalent amount in Experimental Example 55. Panels (a), (b), and (c) show the results of measuring the antibody concentrations in plasma, brain tissue, and CSF, respectively.

FIG. 56 shows the results of blood tests when respective antibodies (BR10, BR802, and BR803) were administered to hTfR-KI (hetero) mice at a dose of 10 mg/kg, calculated as an IgG equivalent amount in Experimental Example 56. Panels (a) and (b) show the results of measuring reticulocyte and iron, respectively.

FIG. 57A shows the measuring results of antibody concentrations in plasma when respective antibodies (BR10, BR802, and BR803) were administered to APP KI/hTfR-KI (homo/hetero) mice at a dose of 10 mg/kg or 30 mg/kg, calculated as an IgG equivalent amount in Experimental Example 57.

FIG. 57B shows the results of quantification of oligomer Aβ, Aβ protofibril in brain tissue when respective antibodies (BR10, BR802, and BR803) were administered to APP KI/hTfR-KI (homo/hetero) mice at a dose of 10 mg/kg or 30 mg/kg, calculated as an IgG equivalent amount in Experimental Example 57. Panels (a), (b) and (c) show the results of oligomer Aβ in 16k_sup, oligomer Aβ in 16k/100k_sup and Aβ protofibril in 16k_sup, respectively.

FIG. 58A shows the pharmacokinetic profile of BR802 and BR803 in serum and CSF when BR802 and BR803 were administered to male cynomolgus monkeys at a dose of 5 mg/kg and 30 mg/kg in Experimental Example 58.

FIG. 58B shows the results of blood tests when BR802 and BR803 were administered to male cynomolgus monkeys at a dose of 5 mg/kg and 30 mg/kg in Experimental Example 58. Panels (a) and (b) show the results of measuring reticulocyte and iron, respectively.

FIG. 59A shows the distribution in the brain and the binding to amyloid plaque 1 day after the respective antibodies (BR10, BR802, and BR803) were administered to APP KI/hTfR-KI (homo/hetero) mice at a dose of 10 mg/kg, calculated as an IgG equivalent amount in Experimental Example 59.

FIG. 59B shows the distribution in the brain and the binding to amyloid plaque 3 days after the respective antibodies (BR10, BR802, and BR803) were administered to APP KI/hTfR-KI (homo/hetero) mice at a dose of 10 mg/kg, calculated as an IgG equivalent amount in Experimental Example 59.

FIG. 59C shows the distribution in the brain and the binding to amyloid plaque 7 days after the respective antibodies (BR10, BR802, and BR803) were administered to APP KI/hTfR-KI (homo/hetero) mice at a dose of 10 mg/kg, calculated as an IgG equivalent amount in Experimental Example 59.

FIG. 59D shows the distribution in the brain and the binding to amyloid plaque 14 days after the respective antibodies (BR10, BR802, and BR803) were administered to APP KI/hTfR-KI (homo/hetero) mice at a dose of 10 mg/kg, calculated as an IgG equivalent amount in Experimental Example 59.

FIG. 60 shows the results of evaluating the ARIA risks by immune cell infiltration when BR847 and BR803 were administered to 5xFAD/hTfR-KI (hemi/homo) mice in Experimental Example 60.

FIG. 61 shows schematic diagrams of BR11 and BR586 in Experimental Example 61.

FIG. 62A shows the results of antibody concentrations in plasma when (a) BR11 and (b) BR586 were administered to APP KI/hTfR-ApD-KI (homo/homo) mice for 12 weeks in Experimental Example 62.

FIG. 62B shows the results of quantifying p-tau in brain sections when BR11 and BR586 were administered to APP KI/hTfR-ApD-KI (homo/homo) mice for 12 weeks in Experimental Example 62. FIG. 62B shows the quantification results of phosphorylated tau231 (pTau231) area.

FIG. 62C shows the amyloid plaques in brain (images) when BR11 and BR586 were administered to APP KI/hTfR-ApD-KI (homo/homo) mice for 12 weeks in Experimental Example 62.

FIG. 62D shows the results of quantifying amyloid plaque area when BR11 and BR586 were administered to APP KI/hTfR-ApD-KI (homo/homo) mice for 12 weeks in Experimental Example 62.

FIG. 62E shows the results of quantification of oligomer Aβ, insoluble Aβ, pyroglutamate Aβ, and Aβ protofibril in brain tissue when BR11 and BR586 were administered to APP KI/hTfR-ApD-KI (homo/homo) mice for 12 weeks in Experimental Example 62. FIG. 62E shows the results of (a) oligomer Aβ in 16k_sup, (b) insoluble Aβ in 16k_ppt, (c) pyroglutamate Aβ in 16k_ppt, (d) oligomer Aβ in 16k/100k_sup, (e) insoluble Aβ in 16k/100k_ppt, (f) pyroglutamate Aβ in 16k/100k_ppt, and (g) Aβ protofibril in 16k_sup, respectively.

FIG. 63 shows schematic diagrams of BR838 and BR839 in Experimental Example 63.

FIG. 64A shows the results of antibody concentrations in plasma when (a) BR11 and (b) BR838 were administered to APP KI/hTfR-KI (homo/hetero) mice for 12 weeks in Experimental Example 64.

FIG. 64B shows the amyloid plaques in brain (images) when BR11 and BR838 were administered to APP KI/hTfR-KI (homo/hetero) mice for 12 weeks in Experimental Example 64.

FIG. 64C shows the results of quantifying amyloid plaque area when BR11 and BR838 were administered to APP KI/hTfR-KI (homo/hetero) mice for 12 weeks in Experimental Example 64.

FIG. 64D shows the results of quantification of oligomer Aβ, insoluble Aβ, pyroglutamate Aβ, and Aβ protofibril in brain tissue when BR11 and BR838 were administered to APP KI/hTfR-KI (homo/hetero) mice for 12 weeks in Experimental Example 64. FIG. 64D shows the results of (a) oligomer Aβ in 16k_sup, (b) insoluble Aβ in 16k_ppt, (c) pyroglutamate Aβ in 16k_ppt, (d) oligomer Aβ in 16k/100k_sup, (e) insoluble Aβ in 16k/100k_ppt, (f) pyroglutamate Aβ in 16k/100k_ppt, and (g) Aβ protofibril in 16k_sup, respectively.

FIG. 65 shows the results of comparing antibody productivity of stable cell lines established in Experimental Examples 25, 29, and 40.

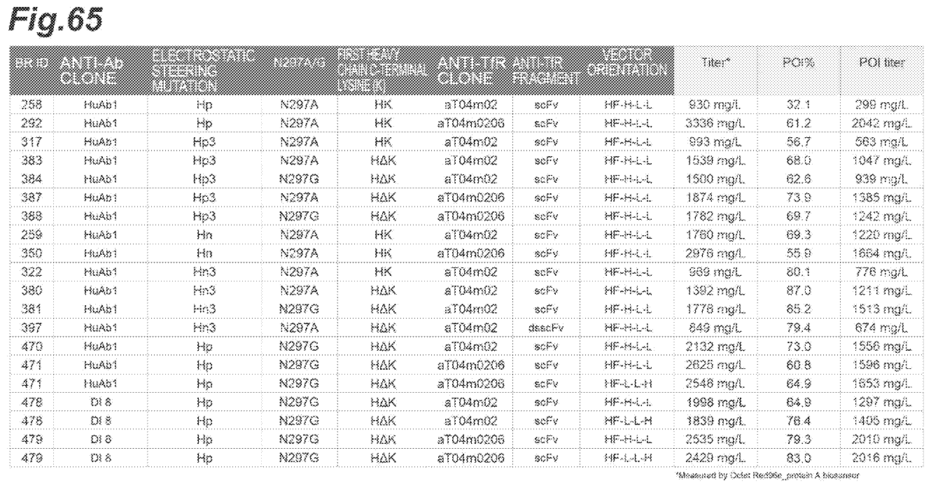

FIG. 66 shows schematic diagrams of BR834, BR835, BR836 and BR837 in Experimental Example 66.

FIG. 67 shows schematic diagrams of the bispecific antibody of the present invention.

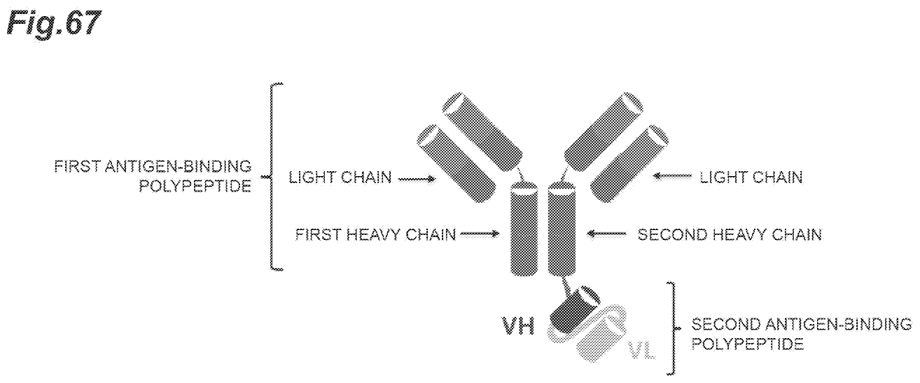

### DETAILED DESCRIPTION

### [Definition]

The term “antibody” used herein means immunoglobulin, a protein that specifically binds to an antigen, unless otherwise stated. The immunoglobulin is not particularly limited, and may be derived from any of commonly known isotypes, including IgA, secretory IgA, IgG, IgE, and IgM. Generally, the immunoglobulin is composed of two heavy chains and two light chains linked to each other by disulfide bonds (in the present specification, also referred to as “four-chain antibody”). Each heavy chain includes a heavy chain variable region (VH) and a heavy chain constant region (CH), and the heavy chain constant region includes three constant domains: CH1, CH2, and CH3. Each light chain includes a light chain variable region (VL) and a light chain constant region (CL), and the light chain constant region includes one constant domain: CL. The VH and VL regions include framework regions (FRs) and complementarity-determining regions (CDRs), each includes three CDRs and four FRs, namely, FR1, CDR1, FR2, CDR2, FR3, CDR3, and FR4, in this order from the N-terminus to the C-terminus. The heavy and light chain variable regions include a binding domain that interact with an antigen. In the present specification, as a technique for determining CDRs, the Kabat numbering system (Kabat et al, Sequences of Proteins of Immunological Interest, 5th ed., 1991, National Institutes of Health, Bethesda MD), which is an approach based on cross-species sequence variability, is used.

In the antibody as used herein, not only a monospecific antibody but also a multispecific antibody (including bispecific antibody, trispecific antibody, and the like) are included. The “bispecific antibody” means an antibody that simultaneously binds to two different antigens or two different epitopes in the same antigen. The bispecific antibody may include two different antigen-binding polypeptides that each bind to two different antigens or two different epitopes in the same antigen. The antigen-binding polypeptide may be an antibody or antigen-binding fragment, which will be described later. In the bispecific antibody, the two different antigen-binding polypeptides are connected to each other, and for example, one antigen-binding polypeptide is fused to the C-terminus or N-terminus of the other antigen-binding polypeptide.

The antibody may also be a human antibody, a humanized antibody, or a chimeric antibody, unless otherwise stated. In one embodiment, the antibody is a monoclonal antibody.

The “human antibody” used herein refers to an antibody in which its entirety is encoded by a human-derived gene. However, an antibody encoded by a gene obtained by introducing a mutation to the original human gene for the purpose of enhancing the expression efficiency of the gene, or the like, is also a human antibody. In addition, an antibody obtained by combining two or more genes that encode a human antibody, and replacing a part of a human antibody with a part of another human antibody is also a human antibody. The human antibody has complementarity-determining regions (CDRs) at three locations on immunoglobulin light chains and complementarity-determining regions (CDRs) at three locations on immunoglobulin heavy chains. The three CDRs on the immunoglobulin light chains are referred to as LCDR1, LCDR2, and LCDR3, in the order from the one on the N-terminal side. The three CDRs on the immunoglobulin heavy chains are referred to as HCDR1, HCDR2, and HCDR3, in the order from the one on the N-terminal side.

The “humanized antibody” used herein refers to an antibody in which the amino acid sequence of a part of the variable region (e.g., in particular, all or a part of CDRs) is derived from a non-human mammal, and the rest of the region is derived from a human. Examples of the humanized antibody include an antibody produced by replacing complementarity-determining regions (CDRs) at three locations on immunoglobulin light chains and complementarity-determining regions (CDRs) at three locations on immunoglobulin heavy chains, the light and heavy chains composing a human antibody, with CDRs of another mammal. The biological species of other mammals from which the CDR to be transplanted to the appropriate position of the human antibody is not particularly limited as long as it is a non-human mammal, and examples thereof include a mouse, a rat, a rabbit, a horse, or a non-human primate, for example, a mouse or a rat, and for example, a mouse.

The “chimeric antibody” used herein refers to an antibody obtained by linking fragments of two or more different antibodies derived from two or more different species. Examples of the chimeric antibody include a chimeric antibody between a human antibody and an antibody of another mammal, and such an antibody is obtained by replacing a part of the human antibody with a part of the antibody of a non-human mammal. Specific examples of such chimeric antibodies include chimeric antibodies in which the Fc region is derived from a human antibody, whereas the Fab region is derived from an antibody of another mammal. By contrast, those in which the Fc region is derived from an antibody of another mammal, whereas the Fab region is derived from a human antibody are also chimeric antibodies. The hinge region can be derived from either the human antibody or the antibody of another mammals.

Further, the chimeric antibody may be composed of variable regions and constant regions. Other specific examples of the chimeric antibodies include those in which the heavy chain constant regions (CH) and the light chain constant regions (CL) are derived from a human antibody, whereas the heavy chain variable regions (VH) and the light chain variable regions (VL) are derived from an antibody of another mammal, and by contrast, those in which the heavy chain constant regions (CH) and the light chain constant regions (CL) are derived from an antibody of another mammal, whereas the heavy chain variable regions (VH) and the light chain variable regions (VL) are derived from a human antibody. Here, the biological species of another mammal is not particularly limited as long as it is a non-human mammal, and examples thereof include a mouse, a rat, a rabbit, a horse, or a non-human primate.

The “antigen-binding fragment” used herein refers to a fragment of an antibody that retains at least a part of the specific binding activity to an antigen, and can include a complementarity-determining region (CDR). In the antigen-binding fragment, a heavy chain antibody, a light chain antibody, VHH, VNAR, and those in which a part thereof is deleted are included. Examples of the antigen-binding fragment can include a double-chain antibody consisting of a heavy chain and a light chain, a single-chain antibody obtained by connecting a heavy chain with a light chain via a peptide linker, a scFv (single-chain Fv) and a dsscFv (disulfide-stabilized single-chain Fv), which are single-chain antibodies obtained by connecting a heavy chain variable region with a light chain variable region via a peptide linker, as well as Fab, F(ab′) and F(ab′)2, which are obtained by deleting the Fc region from a four-chain antibody.

The “linker” used herein includes a chemical linker (non-peptide linker) and a peptide linker described later. The “chemical linker” can be connected via a free amino, imino, hydroxyl, thiol, or a carboxyl group. In one embodiment, the first antigen-binding polypeptide or second antigen-binding polypeptide of the present disclosure is connected to such a chemical linker by one or more chemically active groups of the amino acid residues included in the polypeptide. The chemically active groups may be included in the natural amino acid residues in the amino acid sequence of the first antigen-binding polypeptide or second antigen-binding polypeptide, or, for example, may be introduced by a DNA recombinant technique (e.g., by introducing a cysteine residue to the amino acid sequence) or by protein biochemistry (e.g., by reduction, pH adjustment, or hydrolysis). As a chemical linker, linkers used for antibody-drug conjugates can be used. The chemical linker is not particularly limited, and examples thereof include ε-aminocaproic acid, β amino alanine, γ-aminobutyric acid, 7-aminoheptanoic acid, 12-aminolauric acid, glutamic acid, p-amino benzoic acid, 4-maleimidobutyric acid N-succinimidyl (GMBS), 6-maleimidohexanoic acid N-succinimidyl (EMCS), 3-(2-pyridyldithio) propionic acid N-succinimidyl (SPDP), 3-maleimidopropionic acid N-succinimidyl (BMPS), and 4-(N-maleimidomethyl)cyclohexanecarboxylate N-succinimidyl (SMCC).

The “peptide linker” used herein refers to those made of a peptide chain in which two or more amino acids are connected by peptide bonds. The N-terminus of the peptide linker is connected to the C-terminus of a first protein, by a peptide bond, and the C-terminus of the peptide linker is connected to the N-terminus of a second protein by a peptide bond, and thereby two proteins can be connected to each other by linkers. Examples of the peptide linker include a linker consisting of 1 to 50, 1 to 20, or 1 to 10 amino acids, or a linker consisting of 15 to 20 amino acids, for example, a peptide linker consisting of 1 to 5 or 1 to 6 naturally-occurring L-amino acids or its repeated sequences. In one embodiment, examples of the peptide linker include, but not limited to, glycine-serine linker (also referred to as Gly-Ser linker, or GS linker) composed of glycine and serine, and a linker composed of glycine, serine, and other amino acids. Specifically, examples thereof can include linkers including GS, GGS, GGGS (SEQ ID NO: 74), GGGGS (SEQ ID NO: 75), APGSYTGSAPG (SEQ ID NO: 76), or their repeated sequences. Further specific examples thereof can include (GGS)n, (GGGS)n, (GGGGS)n, a linker with (GGGGS)n linked at the C-terminus of GS, a linker with GGGG (SEQ ID NO: 77) linked at the C-terminus of (GGGGS)n (for example, GGGGSGGGGSGGGG (SEQ ID NO: 287), GGGGGGGGSGGGGSGGGGSGGGG (SEQ ID NO: 288)), GGGGSGGGGSGGGGS (SEQ ID NO: 78), GGGGSAAA (SEQ ID NO: 79), GGGGSGGGGSGGGGSAAA (SEQ ID NO: 255) (wherein n is 1, 2, 3, 4, 5, or 6). When a plurality of peptide linkers are included in the antibody (including the bispecific antibody) or the antigen-binding fragment of the present disclosure, these may be the same or different.

The “scFv” or “single-chain Fv” used herein refers to a molecule in which the heavy chain variable region (VH) and light chain variable region (VL) are connected by peptide linker. The scFv can have the general structure: NH2-VL-linker-VH-COOH or NH2-VH-linker-VL-COOH. The peptide linker here is as described above.

The “dsscFv” or “disulfide-stabilized single-chain Fv” used herein refers to scFv having disulfide bond between heavy chain variable region (VH) and light chain variable region (VL). For example, in dsscFv, disulfide bond is formed between cysteine residue present in VH and cysteine residue present in VL.

The “Fab” used herein refers to a molecule in which one light chain including a variable region and a CL region (light chain constant region), and one heavy chain including a variable region and a CH1 region are connected to each other by a disulfide bond between respective cysteine residues present therein. In Fab, the heavy chain can include a part of the hinge region in addition to the variable region and CH1 region: however, the hinge region in this case lacks a cysteine residue, which is present in the hinge region to connect heavy chains of the antibody to each other. In Fab, the light and heavy chains are connected to each other by disulfide bonds formed between cysteine residues present in the light chain constant region (CL region) and cysteine residues present in the heavy chain constant region (CH1 region) or the hinge regions. The heavy chain forming Fab is referred to as a Fab heavy chain. Fab lacks a cysteine residue, which is present in the hinge region to connect heavy chains of the antibody to each other, and therefore, Fab consists of one light chain and one heavy chain. The light chains constituting Fab include variable regions and CL regions. The heavy chains constituting Fab may consist of the variable region and CH1 region, or may include a part of the hinge region in addition to the variable region and the CH1 region. Provided that, in this case, the hinge region is selected not to include a cysteine residue, which connects the heavy chains to each other, in order not to form a disulfide bond between the two heavy chains in the hinge region. In F(ab′), the heavy chain thereof includes a full or a part of the hinge region including, in addition to the variable region and CH1 region, a cysteine residue, which connects heavy chains to each other. The F(ab′)2 refers to a molecule in which two F(ab′)s are connected to each other by a disulfide bond between cysteine residues present in the respective hinge regions. The heavy chain forming F(ab′) or F(ab′)2 is referred to as a Fab′ heavy chain.

The “Fc” or “Fc region” used herein refers to a region including a fragment consisting of the CH2 region and CH3 region in the antibody molecule.

The antigen-binding fragment used herein includes scFab, scF(ab′), and scF(ab′)2 in which light chains and heavy chains constituting the above Fab, F(ab′), or F(ab′)2 are connected via a linker sequence to form a single-chain antibody. Here, scFab, scF(ab′), and scF(ab′)2 may be those obtained by connecting a peptide linker to the C-terminus of the light chain and then a heavy chain to the C-terminus of the peptide linker, or may be those obtained by connecting a peptide linker to the C-terminus of the heavy chain and then a light chain to the C-terminus of the peptide linker. Further, scFv or dsscFv in which the light chain variable region and the heavy chain variable region are connected by a peptide linker to form a single-chain antibody, may be obtained by connecting a peptide linker to the C-terminus of the light chain variable region, and then the heavy chain variable region to the C-terminus of the peptide linker, or may be obtained by connecting a peptide linker to the C-terminus of the heavy chain variable region and then the light chain variable region to the C-terminus of the peptide linker. The peptide linker here is as described above.

The “single-chain antibody” used herein refers to a protein formed by connecting the amino acid sequence including all or a part of the immunoglobulin light chain variable region, the peptide linker, and the amino acid sequence including all or a part of the immunoglobulin heavy chain variable region to each other, and that can specifically bind to a certain antigen. The single-chain antibody can include all three CDRs of the immunoglobulin heavy chain and all three CDRs of the immunoglobulin light chain. However, it can be a single-chain antibody in which one or more CDRs are deleted, as long as the antigen-specific affinity of the antibody is maintained.

The “antigen-binding polypeptide” used herein means any polypeptide or protein that has the biological activity to specifically bind to an antigen. The antigen-binding polypeptide may be immunoglobulin that specifically binds to an antigen and can include all three CDRs of the immunoglobulin heavy chain and all three CDRs of the immunoglobulin light chain. The antigen-binding polypeptide encompasses an antibody or an antigen-binding fragment thereof, and a fusion protein.

The “polypeptide” used herein refers to a generic term of a compound in which two or more amino acids are connected by peptide bonds, and includes a peptide and protein.

The term “amyloid beta (Aβ)-related disease” used herein refers to a disease related to the development, aggregation, or accumulation of Aβ in the brain, or a disease characterized by them. In the Aβ-related diseases, Alzheimer's disease (AD), mild cognitive impairment due to AD (MCI due to AD), preclinical AD, and Down's syndrome are included. Preclinical AD refers to a preclinical stage of Alzheimer's disease with normal cognitive function but accompanied by accumulation of Aβ in the brain.

### [Bispecific Antibody]

The bispecific antibody of the present disclosure is a bispecific antibody that binds to amyloid beta (Aβ) and a human transferrin receptor (TfR).

The bispecific antibody in one embodiment includes a bispecific antibody including a first antigen-binding polypeptide that binds to amyloid beta (Aβ) and a second antigen-binding polypeptide that binds to a human transferrin receptor (TfR).

The bispecific antibody in a particular embodiment includes a bispecific antibody including a first antigen-binding polypeptide that binds to amyloid beta (Aβ) protofibril and a second antigen-binding polypeptide that binds to a human transferrin receptor (TfR).

### (Second Antigen-Binding Polypeptide)

The second antigen-binding polypeptide that binds to human transferrin receptor (TfR) of the present disclosure includes an anti-human TfR antibody or an antigen-binding fragment thereof.

The anti-human TfR antibody or antigen-binding fragment thereof in one embodiment includes an antibody that binds to human TfR or an antigen-binding fragment thereof. The antibody that binds to human TfR or an antigen-binding fragment thereof may include the predetermined heavy chain variable region (VH) and the predetermined light chain variable region (VL) described below.

The heavy chain variable region includes:
a heavy chain CDR1 (HCDR1) consisting of the amino acid sequence set forth in SEQ ID NO: 2 (DYAMS);
a heavy chain CDR2 (HCDR2) consisting of the amino acid sequence set forth in SEQ ID NO: 254 (AIX₃X₄X₅X₆X₇X₈TYYADSVKG); and
a heavy chain CDR3 (HCDR3) consisting of the amino acid sequence set forth in SEQ ID NO: 3 (MKSTRHWIDD), the light chain variable region includes:
a light chain CDR1 (LCDR1) consisting of the amino acid sequence set forth in SEQ ID NO: 4 (QGDSLRSYYAS);
a light chain CDR2 (LCDR2) consisting of the amino acid sequence set forth in SEQ ID NO: 5 (GKNNRPS); and
a light chain CDR3 (LCDR3) consisting of the amino acid sequence set forth in SEQ ID NO: 6 (QSYDSSSYHYV), and
   wherein in the above AIX₃X₄X₅X₆X₇X₈TYYADSVKG (SEQ ID NO: 254), X₃ is arginine (R), X₄ is glycine (G) or tryptophan (W), X₅ is histidine (H), X₆ is serine(S), X₇ is glycine (G) or serine(S), and X₈ is tryptophan (W). Here, HCDR2 consisting of the amino acid sequence set forth in SEQ ID NO: 254 may consist of AIRWHX₆X₇WTYYADSVKG (SEQ ID NO: 15). Here, X₆ is serine(S), and X₇ is glycine (G) or serine(S).

In the anti-human TfR antibody or an antigen-binding fragment thereof according to one embodiment, the combination of HCDR1 and HCDR2 is No. 2 or 8 shown in the following Table 1.

TABLE 1

| Combi- | | |
|---|---|---|
| nation | HCDR1 | HCDR2 |
| 1 | SYAMS | AISGSGGSTYYADSVKG |
| | (SEQ ID NO: 1) | (SEQ ID NO: 7) |
| 2 | DYAMS | AIRWHSSWTYYADSVKG |
| | (SEQ ID NO: 2) | (SEQ ID NO: 8) |
| 3 | SYAMS | AIRWHSSWTYYADSVKG |
| | (SEQ ID NO: 1) | (SEQ ID NO: 8) |
| 4 | DYAMS | AISWHSSWTYYADSVKG |
| | (SEQ ID NO: 2) | (SEQ ID NO: 9) |
| 5 | DYAMS | AIRGHSSWTYYADSVKG |
| | (SEQ ID NO: 2) | (SEQ ID NO: 10) |
| 6 | DYAMS | AIRWSSSWTYYADSVKG |
| | (SEQ ID NO: 2) | (SEQ ID NO: 11) |
| 7 | DYAMS | AIRWHGSWTYYADSVKG |
| | (SEQ ID NO: 2) | (SEQ ID NO: 12) |
| 8 | DYAMS | AIRWHSGWTYYADSVKG |
| | (SEQ ID NO: 2) | (SEQ ID NO: 13) |
| 9 | DYAMS | AIRWHSSSTYYADSVKG |
| | (SEQ ID NO: 2) | (SEQ ID NO: 14) |

The anti-human TfR antibody or an antigen-binding fragment thereof in a further embodiment may include the predetermined heavy chain variable region (VH) and the predetermined light chain variable region (VL) described below.

The heavy chain variable region includes:
a heavy chain CDR1 (HCDR1) consisting of the amino acid sequence set forth in SEQ ID NO: 2 (DYAMS);
a heavy chain CDR2 (HCDR2) consisting of the amino acid sequence set forth in SEQ ID NO: 8 (AIRWHSSWTYYADSVKG) or set forth in SEQ ID NO: 13 (AIRWHSGWTYYADSVKG); and
a heavy chain CDR3 (HCDR3) consisting of the amino acid sequence set forth in SEQ ID NO: 3 (MKSTRHWIDD),
   the light chain variable region includes:
a light chain CDR1 (LCDR1) consisting of the amino acid sequence set forth in SEQ ID NO: 4 (QGDSLRSYYAS);
a light chain CDR2 (LCDR2) consisting of the amino acid sequence set forth in SEQ ID NO: 5 (GKNNRPS); and
a light chain CDR3 (LCDR3) consisting of the amino acid sequence set forth in SEQ ID NO: 6 (QSYDSSSYHYV).

The transferrin receptor (TfR) is a transmembrane protein that incorporates a complex of transferrin and iron (Fe) in blood into cells, and present on the surface of cerebrovascular endothelial cells. The transferrin receptor (TfR) used herein may be a human transferrin receptor (also described as “human TfR” or “hTfR”) or a monkey transferrin receptor (also described as “monkey TfR” or “cmTfR”), although the origin from which it is derived is not particularly limited. The human transferrin receptor refers to a transmembrane protein consisting of the amino acid sequence set forth in GenBank Accession No. NP_001121620.1 (SEQ ID NO: 256), and the monkey transferrin receptor refers to a transmembrane protein consisting of the amino acid sequence set forth in GenBank Accession No. XP_045243212 (SEQ ID NO: 257). The second antigen-binding polypeptide that binds to a human transferrin receptor (TfR) in one embodiment is an anti-human TfR antibody or an antigen-binding fragment thereof.

The anti-human TfR antibody or an antigen-binding fragment thereof of the present disclosure specifically binds human TfR and is taken up into cells, for example, into cerebrovascular endothelial cells, by human TfR, followed by being released to the opposite side of the cells by transcytosis. The anti-human TfR antibody or an antigen-binding fragment thereof of the present disclosure includes predetermined HCDR1, HCDR2, HCDR3, LCDR1, LCDR2, and LCDR3, thereby specifically binding to human transferrin receptor (TfR), being taken up into cells by human TfR present on the cell surface and being released to the opposite side of the cells by transcytosis. In the anti-human TfR antibody or an antigen-binding fragment thereof of the present disclosure, the amino acid sequences of HCDR1, HCDR3, LCDR1, LCDR2, and LCDR3 remain unchanged, whereas HCDR2 can be changed. HCDR2 is AIRWHSSWTYYADSVKG (SEQ ID NO: 8) or AIRWHSGWTYYADSVKG (SEQ ID NO: 13).

The anti-human TfR antibody or an antigen-binding fragment thereof of the present disclosure specifically binds to TfR present on the surfaces of cerebrovascular endothelial cells, and exhibits the pH-dependent binding to human TfR. It is considered that the anti-human TfR antibody or an antigen-binding fragment thereof of the present disclosure binds to human TfR on the surfaces of cerebrovascular endothelial cells and then is taken up into the cells by endosome, and when pH in the endosome is decreased, the antibody or an antigen-binding fragment thereof is dissociated from TfR followed by being released to the opposite side of the cerebrovascular endothelial cells (i.e., on the side of brain tissue), thereby being transferred into the brain. The pH-dependency in the present disclosure refers to that the binding capacity to human TfR (e.g., EC50) indicates that dissociation occurs more readily under pH 6.0 conditions compared to pH 7.4 conditions, and therefore, the anti-human TfR antibody or an antigen-binding fragment thereof bound to human TfR at pH 7.4 is taken up into cells, and when the pH is decreased to 6.0, it can be easily dissociated from human TfR. For example, the EC50 under pH 6.0 conditions may be 1.5 times or more, or 1.6 times or more, higher than that under pH 7.4 conditions.

The anti-human TfR antibody or an antigen-binding fragment thereof in one embodiment has a feature of specifically binding not only to human TfR but also to recombinant monkey TfR protein (monkey cross-reactivity). The anti-human TfR antibody or an antigen-binding fragment thereof in one embodiment specifically binds not only to human TfR but also to recombinant monkey TfR protein, and the binding to human TfR and recombinant monkey TfR protein is pH dependent. In one embodiment, the binding capacity to human TfR and recombinant monkey TfR protein (e.g., shown as EC50) indicates that dissociation occurs more readily under pH 6.0 conditions compared to pH 7.4 conditions. In one embodiment, EC50 of the anti-human TfR antibody for human TfR or recombinant monkey TfR protein under pH 6.0 conditions may be 1.5 times or more, or 1.6 times or more, higher than that under pH 7.4 conditions.

The anti-human TfR antibody in one embodiment or an antigen-binding fragment thereof includes:
a heavy chain variable region consisting of the amino acid sequence set forth in SEQ ID NO: 66 or 72; and
a light chain variable region consisting of the amino acid sequence set forth in SEQ ID NO: 65.

The anti-human TfR antibody in a particular embodiment or an antigen-binding fragment thereof includes:
a heavy chain variable region consisting of the amino acid sequence set forth in SEQ ID NO: 66; and
a light chain variable region consisting of the amino acid sequence set forth in SEQ ID NO: 65.

The anti-human TfR antibody in a particular embodiment or an antigen-binding fragment thereof includes:
a heavy chain variable region consisting of the amino acid sequence set forth in SEQ ID NO: 72; and
a light chain variable region consisting of the amino acid sequence set forth in SEQ ID NO: 65.

The anti-human TfR antibody in another embodiment or an antigen-binding fragment thereof includes:
a heavy chain variable region consisting of the amino acid sequence set forth in SEQ ID NO: 267; and
a light chain variable region consisting of the amino acid sequence set forth in SEQ ID NO: 265.

The anti-human TfR antibody in a further embodiment or an antigen-binding fragment thereof includes:
a heavy chain variable region consisting of the amino acid sequence set forth in SEQ ID NO: 275; and
a light chain variable region consisting of the amino acid sequence set forth in SEQ ID NO: 273.

The anti-human TfR antibody in another embodiment or an antigen-binding fragment thereof binds to an amino acid residue corresponding to at least one amino acid residue (predetermined amino acid residue) selected from the group consisting of D245, Y247, P249, E350, G351, D352, C353, P354, S355, S361, M365, E369, and Q721 of the human transferrin receptor (TfR) consisting of the amino acid sequence set forth in SEQ ID NO: 256. These predetermined amino acid residues are, as shown in Experimental Example 11, epitopes determined from the results of analyzing the binding interface between hTfR and aT04m02. Here, the amino acid residue corresponding to the predetermined amino acid residue in the amino acid sequence set forth in SEQ ID NO: 256 refers to an amino acid residue in the amino acid sequence of a target protein (e.g., mutant protein such as human TfR with a mutation, and homologous protein such as TfR derived from monkey), located at a position corresponding to the location of the predetermined amino acid residue of SEQ ID NO: 256, when the alignment is performed to the amino acid sequence of the target protein and the amino acid sequence set forth in SEQ ID NO: 256.

In one embodiment, the anti-human TfR antibody or an antigen-binding fragment thereof binds to amino acid residues corresponding to amino acid residues (predetermined amino acid residues) of D245, Y247, P249, E350, G351, D352, C353, P354, S355, S361, M365, E369, and Q721 of the human transferrin receptor (TfR) consisting of the amino acid sequence set forth in SEQ ID NO: 256.

D245, Y247, P249, E350, G351, D352, C353, P354, S355, S361, M365, E369, and Q721 of the human transferrin receptor (TfR) consisting of the amino acid sequence set forth in SEQ ID NO: 256 are D245, D247, P249, E350, G351, D352, C353, P354, S355, S361, M365, E369, and Q721 of the recombinant monkey transferrin receptor (cmTfR) protein consisting of the amino acid sequence set forth in SEQ ID NO: 257, respectively.

The anti-human TfR antibody in one embodiment or an antigen-binding fragment thereof binds to an amino acid residue corresponding to at least one amino acid residue selected from the group consisting of D352, S355, and S361 of the human transferrin receptor (TfR) consisting of the amino acid sequence set forth in SEQ ID NO: 256.

The anti-human TfR antibody in one embodiment or an antigen-binding fragment thereof binds to amino acid residues corresponding to amino acid residues of D352, S355, and S361 of the human transferrin receptor (TfR) consisting of the amino acid sequence set forth in SEQ ID NO: 256.

D352, S355, and S361 of the human transferrin receptor (TfR) consisting of the amino acid sequence set forth in SEQ ID NO: 256 are D352, S355, and S361 of the recombinant monkey transferrin receptor (cmTfR) protein consisting of the amino acid sequence set forth in SEQ ID NO: 257, respectively.

The second antigen-binding polypeptide that binds to a human transferrin receptor (TfR) in one embodiment is an antigen-binding fragment of an anti-human TfR antibody. The antigen-binding fragment of the anti-human TfR antibody in one embodiment is scFv, dsscFv, Fab, F(ab′), F(ab′)2, scFab, scF(ab′), or scF(ab′)2. Examples of scFv include SEQ ID NOs: 19, 33, 35, 37, 39, 41, 43, 45, and 47, shown in the following Table 2. These scFvs are connected in this order from the N-terminus: VH, a peptide linker: GGGGSGGGGSGGGG (SEQ ID NO: 287), and VL. The scFvs consisting of the amino acid sequences set forth in SEQ ID NOs: 19, 33, 35, 37, 39, 41, 43, 45, and 47 are aT04-scFv, aT04m02-scFv, aT04m0201-scFv, aT04m0202-scFv, aT04m0203-scFv, aT04m0204-scFv, aT04m0205-scFv, aT04m0206-scFv, and aT04m0207-scFv, respectively. These scFvs include any of the combinations of 1 to 9 of HCDR1 and HCDR2 shown in the above Table 1. Among these, the scFv consisting of the amino acid sequence set forth in SEQ ID NO: 33, 35, 43, or 45 has cross-reactivity to recombinant monkey TfR protein. The antigen-binding fragment of the anti-human TfR antibody in one embodiment is scFv. In one embodiment, the scFv includes the amino acid sequence set forth in SEQ ID NO: 33 or 45 shown in the following Table 2. For example, the scFvs consisting of the amino acid sequences set forth in SEQ ID NOs: 33 and 45 are aT04m02-scFv and aT04m0206-scFv in Experimental Examples, respectively. The scFv consisting of the amino acid sequence set forth in SEQ ID NO: 33 or 45 has cross-reactivity to recombinant monkey TfR protein.

The scFv may have VH, a peptide linker: GGGGSGGGGSGGGGSGGGGSGGGG (SEQ ID NO: 288), and VL linked in this order from the N-terminus, for example, SEQ ID NOs: 289 and 290 shown in Table 2 below.

TABLE 2

| SEQ | |
|---|---|
| ID | |
| NO: | Sequence |
| 19 | EVQLLESGGGLVQPGGSLRLSCAASGFTFSSYAMSWVRQAPGKGLEWV |
| | SAISGSGGSTYYADSVKGRFTISRDNSKNTLYLQMNSLRAEDTAVYYCA |
| | KMKSTRHWIDDWGQGTLVTVSSGGGGSGGGGSGGGGSSELTQDPAVSV |
| | ALGQTVRITCQGDSLRSYYASWYQQKPGQAPVLVIYGKNNRPSGIPDRF |
| | SGSSSGNTASLTITGAQAEDEADYYCQSYDSSSYHYVFGGGTKLTVL |
| 33 | EVQLLESGGGLVQPGGSLRLSCAASGFTFSDYAMSWVRQAPGKGLEWV |
| | SAIRWHSSWTYYADSVKGRFTISRDNSKNTLYLQMNSLRAEDTAVYYCA |
| | KMKSTRHWIDDWGQGTLVTVSSGGGGSGGGGSGGGGSSELTQDPAVSV |
| | ALGQTVRITCQGDSLRSYYASWYQQKPGQAPVLVIYGKNNRPSGIPDRF |
| | SGSSSGNTASLTITGAQAEDEADYYCQSYDSSSYHYVFGGGTKLTVL |
| 35 | EVQLLESGGGLVQPGGSLRLSCAASGFTFSSYAMSWVRQAPGKGLEWV |
| | SAIRWHSSWTYYADSVKGRFTISRDNSKNTLYLQMNSLRAEDTAVYYCA |
| | KMKSTRHWIDDWGQGTLVTVSSGGGGSGGGGSGGGGSSELTQDPAVSV |
| | ALGQTVRITCQGDSLRSYYASWYQQKPGQAPVLVIYGKNNRPSGIPDRF |
| | SGSSSGNTASLTITGAQAEDEADYYCQSYDSSSYHYVFGGGTKLTVL |
| 37 | EVQLLESGGGLVQPGGSLRLSCAASGFTFSDYAMSWVRQAPGKGLEWV |
| | SAISWHSSWTYYADSVKGRFTISRDNSKNTLYLQMNSLRAEDTAVYYCA |
| | KMKSTRHWIDDWGQGTLVTVSSGGGGSGGGGSGGGGSSELTQDPAVSV |
| | ALGQTVRITCQGDSLRSYYASWYQQKPGQAPVLVIYGKNNRPSGIPDRF |
| | SGSSSGNTASLTITGAQAEDEADYYCQSYDSSSYHYVFGGGTKLTVL |
| 39 | EVQLLESGGGLVQPGGSLRLSCAASGFTFSDYAMSWVRQAPGKGLEWV |
| | SAIRGHSSWTYYADSVKGRFTISRDNSKNTLYLQMNSLRAEDTAVYYCA |
| | KMKSTRHWIDDWGQGTLVTVSSGGGGSGGGGSGGGGSSELTQDPAVSV |
| | ALGQTVRITCQGDSLRSYYASWYQQKPGQAPVLVIYGKNNRPSGIPDRF |
| | SGSSSGNTASLTITGAQAEDEADYYCQSYDSSSYHYVFGGGTKLTVL |
| 41 | EVQLLESGGGLVQPGGSLRLSCAASGFTFSDYAMSWVRQAPGKGLEWV |
| | SAIRWSSSWTYYADSVKGRFTISRDNSKNTLYLQMNSLRAEDTAVYYCA |
| | KMKSTRHWIDDWGQGTLVTVSSGGGGSGGGGSGGGGSSELTQDPAVSV |
| | ALGQTVRITCQGDSLRSYYASWYQQKPGQAPVLVIYGKNNRPSGIPDRF |
| | SGSSSGNTASLTITGAQAEDEADYYCQSYDSSSYHYVFGGGTKLTVL |
| 43 | EVQLLESGGGLVQPGGSLRLSCAASGFTFSDYAMSWVRQAPGKGLEWV |
| | SAIRWHGSWTYYADSVKGRFTISRDNSKNTLYLQMNSLRAEDTAVYYC |
| | AKMKSTRHWIDDWGQGTLVTVSSGGGGSGGGGSGGGGSSELTQDPAVS |
| | VALGQTVRITCQGDSLRSYYASWYQQKPGQAPVLVIYGKNNRPSGIPDR |
| | FSGSSSGNTASLTITGAQAEDEADYYCQSYDSSSYHYVFGGGTKLTVL |
| 45 | EVQLLESGGGLVQPGGSLRLSCAASGFTFSDYAMSWVRQAPGKGLEWV |
| | SAIRWHSGWTYYADSVKGRFTISRDNSKNTLYLQMNSLRAEDTAVYYC |
| | AKMKSTRHWIDDWGQGTLVTVSSGGGGSGGGGSGGGGSSELTQDPAVS |
| | VALGQTVRITCQGDSLRSYYASWYQQKPGQAPVLVIYGKNNRPSGIPDR |
| | FSGSSSGNTASLTITGAQAEDEADYYCQSYDSSSYHYVFGGGTKLTVL |
| 47 | EVQLLESGGGLVQPGGSLRLSCAASGFTFSDYAMSWVRQAPGKGLEWV |
| | SAIRWHSSSTYYADSVKGRFTISRDNSKNTLYLQMNSLRAEDTAVYYCA |
| | KMKSTRHWIDDWGQGTLVTVSSGGGGSGGGGSGGGGSSELTQDPAVSV |
| | ALGQTVRITCQGDSLRSYYASWYQQKPGQAPVLVIYGKNNRPSGIPDRF |
| | SGSSSGNTASLTITGAQAEDEADYYCQSYDSSSYHYVFGGGTKLTVL |
| 289 | EVQLLESGGGLVQPGGSLRLSCAASGFTFSDYAMSWVRQAPGKGLEWV |
| | SAIRWHSSWTYYADSVKGRFTISRDNSKNTLYLQMNSLRAEDTAVYYCA |
| | KMKSTRHWIDDWGQGTLVTVSSGGGGSGGGGSGGGGSGGGGSGGGGSS |
| | ELTQDPAVSVALGQTVRITCQGDSLRSYYASWYQQKPGQAPVLVIYGKN |
| | NRPSGIPDRFSGSSSGNTASLTITGAQAEDEADYYCQSYDSSSYHYVFG |
| | GGTKLTVL |
| 290 | EVQLLESGGGLVQPGGSLRLSCAASGFTFSDYAMSWVRQAPGKGLEWV |
| | SAIRWHSGWTYYADSVKGRFTISRDNSKNTLYLQMNSLRAEDTAVYYC |
| | AKMKSTRHWIDDWGQGTLVTVSSGGGGSGGGGSGGGGSGGGGSGGGGS |
| | SELTQDPAVSVALGQTVRITCQGDSLRSYYASWYQQKPGQAPVLVIYG |
| | KNNRPSGIPDRFSGSSSGNTASLTITGAQAEDEADYYCQSYDSSSYHY |
| | VFGGGTKLTVL |

The antigen-binding fragment of the anti-human TfR antibody in another embodiment is dsscFv. In a further embodiment, the dsscFv includes the amino acid sequence set forth in SEQ ID NO: 263 or 271 as shown below. These dsscFvs are connected in this order from the N-terminus: VH, a peptide linker: GGGGSGGGGSGGGG (SEQ ID NO: 287), and VL. For example, the dsscFvs consisting of the amino acid sequences set forth in SEQ ID NOs: 263 and 271 are aT04m02-dsscFv and aT04m0206-dsscFv in Experimental Examples, respectively. The dsscFvs consisting of the amino acid sequences set forth in SEQ ID NO: 263 and 271 have cross-reactivity to recombinant monkey TfR protein.

SEQ ID NO: 263:
EVQLLESGGGLVQPGGSLRLSCAASGFTFSDYAMSWVRQAPGKCL
EWVSAIRWHSSWTYYADSVKGRFTISRDNSKNTLYLQMNSLRAED
TAVYYCAKMKSTRHWIDDWGQGTLVTVSSGGGGSGGGGSGGGGSS
ELTQDPAVSVALGQTVRITCQGDSLRSYYASWYQQKPGQAPVLVI
YGKNNRPSGIPDRFSGSSSGNTASLTITGAQAEDEADYYCQSYDS
SSYHYVFGCGTKLTVL
SEQ ID NO: 271:
EVQLLESGGGLVQPGGSLRLSCAASGFTFSDYAMSWVRQAPGKCL
EWVSAIRWHSGWTYYADSVKGRFTISRDNSKNTLYLQMNSLRAED
TAVYYCAKMKSTRHWIDDWGQGTLVTVSSGGGGSGGGGSGGGGSS
ELTQDPAVSVALGQTVRITCQGDSLRSYYASWYQQKPGQAPVLVI
YGKNNRPSGIPDRFSGSSSGNTASLTITGAQAEDEADYYCQSYDS
SSYHYVFGCGTKLTVL

The dsscFv may have VH, a peptide linker: GGGGSGGGGSGGGGSGGGGSGGGG (SEQ ID NO: 288), and VL linked in this order from the N-terminus, for example, dsscFv comprising the amino acid sequences set forth in SEQ ID NOs: 291 or 292 as shown below.

SEQ ID NO: 291:
EVQLLESGGGLVQPGGSLRLSCAASGFTFSDYAMSWVRQAPGKCL
EWVSAIRWHSSWTYYADSVKGRFTISRDNSKNTLYLQMNSLRAED
TAVYYCAKMKSTRHWIDDWGQGTLVTVSSGGGGSGGGGSGGGGSG
GGGSGGGGSSELTQDPAVSVALGQTVRITCQGDSLRSYYASWYQQ
KPGQAPVLVIYGKNNRPSGIPDRFSGSSSGNTASLTITGAQAEDE
ADYYCQSYDSSSYHYVFGCGTKLTVL
SEQ ID NO: 292:
EVQLLESGGGLVQPGGSLRLSCAASGFTFSDYAMSWVRQAPGKCL
EWVSAIRWHSGWTYYADSVKGRFTISRDNSKNTLYLQMNSLRAED
TAVYYCAKMKSTRHWIDDWGQGTLVTVSSGGGGSGGGGSGGGGSG
GGGSGGGGSSELTQDPAVSVALGQTVRITCQGDSLRSYYASWYQQ
KPGQAPVLVIYGKNNRPSGIPDRFSGSSSGNTASLTITGAQAEDE
ADYYCQSYDSSSYHYVFGCGTKLTVL

The anti-human TfR antibody or an antigen-binding fragment thereof of the present disclosure may be an antibody specifically binding to human TfR and taken up into cells by human TfR, as described above. Therefore, the anti-human TfR antibody or an antigen-binding fragment thereof of the present disclosure can be used as a transport module capable of transporting across the blood-brain barrier (BBB). In other words, a compound that cannot cross or have difficulty in crossing the blood-brain barrier (BBB) alone is transferred into the brain by human TfR when bound to the anti-human TfR antibody or an antigen-binding fragment thereof of the present disclosure, enabling it to show the bioactivity in the brain.

The second antigen-binding polypeptide that binds to a human transferrin receptor (TfR) of the present disclosure may include a heavy chain variable region (VH) including HCDR1 consisting of the amino acid sequence set forth in SEQ ID NO: 2, HCDR2 consisting of the amino acid sequence set forth in SEQ ID NO: 8 or 13, and HCDR3 consisting of the amino acid sequence set forth in SEQ ID NO: 3, and a light chain variable region (VL) including LCDR1 consisting of the amino acid sequence set forth in SEQ ID NO: 4, LCDR2 consisting of the amino acid sequence set forth in SEQ ID NO: 5, and LCDR3 consisting of the amino acid sequence set forth in SEQ ID NO: 6. In one embodiment, HCDR2 consists of the amino acid sequence set forth in SEQ ID NO: 8. In another embodiment, HCDR2 consists of the amino acid sequence set forth in SEQ ID NO: 13.

The second antigen-binding polypeptide that binds to a human transferrin receptor (TfR) in one embodiment includes HCDR2 consisting of the amino acid sequence set forth in SEQ ID NO: 8.

The second antigen-binding polypeptide that binds to a human transferrin receptor (TfR) in one embodiment includes a heavy chain variable region consisting of the amino acid sequence set forth in SEQ ID NO: 66 and a light chain variable region consisting of the amino acid sequence set forth in SEQ ID NO: 65.

The second antigen-binding polypeptide that binds to a human transferrin receptor (TfR) in one embodiment includes a heavy chain variable region consisting of the amino acid sequence set forth in SEQ ID NO: 267 and a light chain variable region consisting of the amino acid sequence set forth in SEQ ID NO: 265.

The second antigen-binding polypeptide that binds to a human transferrin receptor (TfR) in another embodiment binds to at least one amino acid residue selected from the group consisting of D352, S355, and S361 of the human transferrin receptor (TfR) consisting of the amino acid sequence set forth in SEQ ID NO: 256.

The second antigen-binding polypeptide that binds to a human transferrin receptor (TfR) in one embodiment binds to amino acid residues of D352, S355, and S361 of the human transferrin receptor (TfR) consisting of the amino acid sequence set forth in SEQ ID NO: 256.

The second antigen-binding polypeptide that binds to a human transferrin receptor (TfR) in another embodiment includes HCDR2 consisting of the amino acid sequence set forth in SEQ ID NO: 13.

The second antigen-binding polypeptide that binds to a human transferrin receptor (TfR) in a further embodiment includes a heavy chain variable region consisting of the amino acid sequence set forth in SEQ ID NO: 72 and a light chain variable region consisting of the amino acid sequence set forth in SEQ ID NO: 65.

The second antigen-binding polypeptide that binds to a human transferrin receptor (TfR) in a further embodiment includes a heavy chain variable region consisting of the amino acid sequence set forth in SEQ ID NO: 275 and a light chain variable region consisting of the amino acid sequence set forth in SEQ ID NO: 273.

The second antigen-binding polypeptide of the present disclosure may be a full-length antibody, double-chain antibody, or single-chain antibody, and it is, for example, scFv (single-chain Fv) or dsscFv (disulfide-stabilized single-chain Fv).

The second antigen-binding polypeptide in one embodiment is scFv consisting of the amino acid sequences set forth in SEQ ID NOs: 33 or 45.

The second antigen-binding polypeptide in another embodiment is dsscFv consisting of the amino acid sequence set forth in SEQ ID NO: 263 or 271.

The second antigen-binding polypeptide in a specific embodiment is scFv consisting of the amino acid sequences set forth in SEQ ID NOs: 289 or 290.

The second antigen-binding polypeptide in a specific embodiment is dsscFv consisting of the amino acid sequence set forth in SEQ ID NO: 291 or 292.

One embodiment provides a nucleic acid (DNA) that encodes the second antigen-binding polypeptide of the present disclosure. Examples of the nucleic acid that encodes scFvs (aT04m02-scFv and aT04m0206-scFv) consisting of the amino acid sequences set forth in SEQ ID NOs: 33 and 45 includes SEQ ID NOs: 32 and 44, respectively. Further, examples of the nucleic acid that encodes dsscFvs (aT04m02-dsscFv and aT04m0206-dsscFv) consisting of the amino acid sequences set forth in SEQ ID NOs: 263 and 271 include SEQ ID NOs: 262 and 270, respectively.

One embodiment provides a vector including a nucleic acid (DNA) that encodes the second antigen-binding polypeptide of the present disclosure, and further provides a recombinant cell in which the vector has been introduced into a host cell. The vector and host cell are not particularly limited as long as they are those commonly used, and examples thereof include those described later.

The second antigen-binding polypeptide of the present disclosure may be produced by artificial synthesis, or can also be obtained by producing in the above recombinant cells to collect and purify. In addition, for easier purification or easier assay detection, it can be produced as a tag (e.g., His tag) or a fusion protein fused with a detectable marker protein (e.g., GFP).

### (First Antigen-Binding Polypeptide)

The antigen-binding polypeptide that binds to amyloid beta (Aβ) of the present disclosure includes an anti-amyloid beta (Aβ) antibody or an antigen-binding fragment thereof.

The first antigen-binding polypeptide in one embodiment includes a first heavy chain and a second heavy chain including predetermined heavy chain CDR1 (HCDR1), heavy chain CDR2 (HCDR2), and heavy chain CDR3 (HCDR3), and two light chains including light chain CDR1 (LCDR1), light chain CDR2 (LCDR2), and light chain CDR3 (LCDR3); and specifically, the above first heavy chain and second heavy chain each include HCDR1 consisting of the amino acid sequence set forth in SEQ ID NO: 232, HCDR2 consisting of the amino acid sequence set forth in SEQ ID NO: 233, and HCDR3 consisting of the amino acid sequence set forth in SEQ ID NO: 234, and the above two light chains each include LCDR1 consisting of the amino acid sequence set forth in SEQ ID NO: 235, LCDR2 consisting of the amino acid sequence set forth in SEQ ID NO: 236, and LCDR3 consisting of the amino acid sequence set forth in SEQ ID NO: 237.

In another embodiment, those including the heavy chain variable region and light chain variable region of BAN2401 (referring to the anti-Aβ protofibril antibody BAN2401 disclosed in WO2018/081460) are excluded from the first antigen-binding polypeptide. For example, in the first antigen-binding polypeptide, from (a) those in which the first heavy chain and the second heavy chain each include HCDR1 consisting of the amino acid sequence set forth in SEQ ID NO: 232, HCDR2 consisting of the amino acid sequence set forth in SEQ ID NO: 233, and HCDR3 consisting of the amino acid sequence set forth in SEQ ID NO: 234, and the two light chains each include LCDR1 consisting of the amino acid sequence set forth in SEQ ID NO: 235, LCDR2 consisting of the amino acid sequence set forth in SEQ ID NO: 236, and LCDR3 consisting of the amino acid sequence set forth in SEQ ID NO: 237, (b) those in which the first heavy chain and the second heavy chain each include a heavy chain variable region consisting of the amino acid sequence set forth in SEQ ID NO: 285, and the two light chains each include a light chain variable region consisting of the amino acid sequence set forth in SEQ ID NO: 286 are excluded.

Amyloid beta (Aβ) is a peptide derived from an amyloid precursor protein (APP) truncated by β-secretase and γ-secretase, and examples thereof include Aβ40 in which the C-terminus ends at position 40, and Aβ42 in which the C-terminus ends at position 42. In particular, Aβ42 is known that the aggregation propensity is high; the decrease in concentration of cerebrospinal fluid Aβ42 and the increase in Aβ40/42 ratio are observed in Alzheimer's disease; and it exhibits a strong correlation with the amount of amyloid accumulation measured by amyloid PET. It is known that Aβ is present in various conformational states such as a monomer, oligomer, protofibril, and insoluble fibril. In particular, the Aβ protofibril refers to a soluble Aβ aggregate. The origin from which amyloid beta (Aβ) used herein is derived is not particularly limited, but it may be human amyloid beta (Aβ).

In one embodiment, the first antigen-binding polypeptide that binds to amyloid beta (Aβ) of the present disclosure is an anti-Aβ protofibril antibody or an antigen-binding fragment thereof.

The anti-amyloid beta (Aβ) antibody or an antigen-binding fragment thereof of the present disclosure can specifically bind to amyloid beta (Aβ), and in particular, can decrease the amount of amyloid in the brain by including predetermined HCDR1, HCDR2, HCDR3, LCDR1, LCDR2, and LCDR3.

In the first antigen-binding polypeptide according to one embodiment, the first heavy chain and the second heavy chain may each include a heavy chain variable region consisting of the amino acid sequence set forth in SEQ ID NO: 238, and the two light chains may each include a light chain variable region consisting of the amino acid sequence set forth in SEQ ID NO: 239.

The anti-Aβ antibody or an antigen-binding fragment thereof in one embodiment is a full-length antibody. The constant region of such anti-Aβ antibody includes a sequence derived from the constant regions of isotypes that can be selected from human IgA, secretory IgA, IgG, IgE, and IgM. In one embodiment, the constant region of the anti-Aβ antibody includes a sequence derived from the constant region of human IgG. The sequence derived from such constant region of human IgG may include a sequence derived from a single subclass (e.g., IgG1 and IgG2), or may include a sequence derived from a plurality of subclasses (e.g., a chimera of the constant domains of IgG1 and IgG2). One or more amino acid mutations can be introduced into the constant region included in the anti-Aβ antibody, as desired.

In one embodiment, the constant region of the first heavy chain and the constant region of the second heavy chain of the first antigen-binding polypeptide may be a human IgG1 heavy chain constant region, and the human IgG1 heavy chain constant region may include a mutation wherein an asparagine residue at position 297 according to EU numbering has been substituted. Here, the mutation of an asparagine residue at position 297 according to EU numbering may be a mutation wherein the residue has been substituted with a glycine residue (N297G).

The constant region of the first heavy chain of the first antigen-binding polypeptide in one embodiment may include mutations wherein a lysine residue at position 409 and a lysine residue at position 439 according to EU numbering have been substituted in the human IgG1 heavy chain constant region. Here, the mutation of a lysine residue at position 409 according to EU numbering may be a mutation wherein the residue has been substituted with an aspartate residue (K409D), the mutation of a lysine residue at position 439 according to EU numbering may be a mutation wherein the residue has been substituted with a glutamic acid residue (K439E).

The constant region of the second heavy chain of the first antigen-binding polypeptide in one embodiment may include mutations wherein an aspartate residue at position 356 and an aspartate residue at position 399 according to EU numbering have been substituted in the human IgG1 heavy chain constant region. Here, the mutation of an aspartate residue at position 356 according to EU numbering may be a mutation wherein the residue has been substituted with a lysine residue (D356K), the mutation of an aspartate residue at position 399 according to EU numbering may be a mutation wherein the residue has been substituted with a lysine residue (D399K).

The lysine residue at the C-terminus of the first heavy chain of the first antigen-binding polypeptide in one embodiment is deleted (HAK).

The constant region of the first heavy chain of the first antigen-binding polypeptide in one embodiment includes the heavy chain constant region consisting of the amino acid sequence set forth in SEQ ID NO: 240. Further, the constant region of the second heavy chain of the first antigen-binding polypeptide in one embodiment includes the heavy chain constant region consisting of the amino acid sequence set forth in SEQ ID NO: 241.

The constant region of light chain of the first antigen-binding polypeptide in one embodiment includes the amino acid sequence set forth in SEQ ID NO: 119.

The first heavy chain of the first antigen-binding polypeptide in one embodiment includes the heavy chain variable region consisting of the amino acid sequence set forth in SEQ ID NO: 238 and the heavy chain constant region consisting of the amino acid sequence set forth in SEQ ID NO: 240.

The second heavy chain of the first antigen-binding polypeptide in one embodiment includes the heavy chain variable region consisting of the amino acid sequence set forth in SEQ ID NO: 238 and the heavy chain constant region consisting of the amino acid sequence set forth in SEQ ID NO: 241.

The light chain of the first antigen-binding polypeptide in one embodiment includes the light chain variable region consisting of the amino acid sequence set forth in SEQ ID NO: 239 and the light chain constant region consisting of the amino acid sequence set forth in SEQ ID NO: 119.

One embodiment provides a nucleic acid (DNA) that encodes the first antigen-binding polypeptide of the present disclosure. Examples of the nucleic acid that encodes the heavy chain variable region consisting of the amino acid sequence set forth in SEQ ID NO: 238 and the light chain variable region consisting of the amino acid sequence set forth in SEQ ID NO: 239 includes SEQ ID NOs: 278 and 279, respectively. In addition, examples of the nucleic acid that encodes the first heavy chain constant region of the first antigen-binding polypeptide consisting of the amino acid sequence set forth in SEQ ID NO: 240 and the second heavy chain heavy chain constant region of the first antigen-binding polypeptide consisting of the amino acid sequence set forth in SEQ ID NO: 241 includes SEQ ID NOs: 280 and 281, respectively. Further, examples of the nucleic acid that encodes the light chain constant region consisting of the amino acid sequence set forth in SEQ ID NOs: 119 includes SEQ ID NO: 282.

One embodiment provides a vector including a nucleic acid (DNA) that encodes the first antigen-binding polypeptide of the present disclosure, and further provides a recombinant cell in which the vector has been introduced into a host cell. The vector and host cell are not particularly limited as long as they are those commonly used, and examples thereof include those described later.

The first antigen-binding polypeptide of the present disclosure may be produced by artificial synthesis, and can also be obtained by expressing in the above recombinant cells to collect and purify. In addition, for easier purification or easier detection by assay, it can be produced as a fusion protein with a tag (e.g., His tag) or a detectable marker protein (e.g., GFP).

### (Bispecific Antibody)

The bispecific antibody of the present disclosure is a bispecific antibody that binds to amyloid beta (Aβ) and a human transferrin receptor (TfR).

The bispecific antibody in one embodiment is a bispecific antibody that binds to amyloid beta (Aβ) protofibril and a human transferrin receptor (TfR).

The bispecific antibody in one embodiment includes the first antigen-binding polypeptide that binds to amyloid beta (Aβ) and the second antigen-binding polypeptide that binds to a human transferrin receptor (TfR). Here, the first antigen-binding polypeptide and the second antigen-binding polypeptide are as described above. In the bispecific antibody of the present disclosure, the first antigen-binding polypeptide and the second antigen-binding polypeptide are connected to each other. Here, the first antigen-binding polypeptide and the second antigen-binding polypeptide may be directly connected to each other, or may be connected to each other indirectly via a linker. For example, the linker is a peptide linker. Here, the peptide linker is not particularly limited as long as the antigen binding activity and bioactivity of the first antigen-binding polypeptide and the second antigen-binding polypeptide are maintained, and for example, it may be the peptide linker described above, and more specifically, it may be, for example, GGGGSAAA (SEQ ID NO: 79). When connected directly, or when connected via a peptide linker, the first antigen-binding polypeptide and the second antigen-binding polypeptide form a fusion protein.

In the bispecific antibody according to one embodiment, the first antigen-binding polypeptide, a peptide linker if any, and the second antigen-binding polypeptide may be connected in this order from the N-terminus, or conversely, the second antigen-binding polypeptide, a peptide linker if any, and the first antigen-binding polypeptide may be connected in this order from the N-terminus. When the anti-TfR antibody, which is the second antigen-binding polypeptide, or an antigen-binding fragment thereof is an antibody consisting of plural chains (double-chain, triple-chain, or four-chain antibody), the first antigen-binding polypeptide can be connected to any one portion among the plural chains, and can also be connected to a plurality of portions. By contrast, when the anti-amyloid beta (Aβ) antibody, which is the first antigen-binding polypeptide, or an antigen-binding fragment thereof is an antibody consisting of plural chains (double-chain, triple-chain, or four-chain antibody), the second antigen-binding polypeptide can be connected to any one portion among the plural chains, and can also be connected to a plurality of portions.

In one embodiment, the second antigen-binding polypeptide is connected to the heavy chain constant region of the first antigen-binding polypeptide. In one embodiment, the second antigen-binding polypeptide is connected to the Fc region of the first antigen-binding polypeptide. In one embodiment, the second antigen-binding polypeptide is connected to the CH3 domain of the first antigen-binding polypeptide. In one embodiment, the second antigen-binding polypeptide is connected to the C-terminus of the heavy chain constant region of the first antigen-binding polypeptide. In one embodiment, the second antigen-binding polypeptide is an antigen-binding fragment and connected to the C-terminus of the heavy chain constant region of the first antigen-binding polypeptide. In one embodiment, the second antigen-binding polypeptide is scFv or dsscFv and connected to the C-terminus of the first antigen-binding polypeptide via a peptide linker. In one embodiment, the second antigen-binding polypeptide is an antigen-binding fragment (e.g., scFv or dsscFv) and connected to the C-terminus of one of the heavy chains of the first antigen-binding polypeptide via a peptide linker (i.e., one molecule of the second antigen-binding polypeptide binds to one antibody molecule of the first antigen-binding polypeptide).

In the bispecific antibody according to one embodiment, the second antigen-binding polypeptide is connected to the C-terminus of the constant region of the second heavy chain of the first antigen-binding polypeptide. In this case, the lysine residue at the C-terminus of the first heavy chain of the first antigen-binding polypeptide is deleted (HΔK).

### N297G

In the bispecific antibody according to one embodiment, the constant region of the first heavy chain and the constant region of the second heavy chain of the first antigen-binding polypeptide each include a mutation wherein an asparagine residue at position 297 according to EU numbering has been substituted with a glycine residue (N297G). The bispecific antibody including the first antigen-binding polypeptide including the N297G mutation in the heavy chain constant region, and the second antigen-binding polypeptide including the heavy chain variable region including HCDR1 consisting of the amino acid sequence set forth in SEQ ID NO: 2, HCDR2 consisting of the amino acid sequence set forth in SEQ ID NO: 8 or SEQ ID NO: 13, and HCDR3 consisting of the amino acid sequence set forth in SEQ ID NO: 3, and the light chain variable region including LCDR1 consisting of the amino acid sequence set forth in SEQ ID NO: 4, LCDR2 consisting of the amino acid sequence set forth in SEQ ID NO: 5, and LCDR3 consisting of the amino acid sequence set forth in SEQ ID NO: 6 has a lowered binding activity to a Fcγ receptor (FcγRI, FcγRIIA, and FcγRIIIA) and C1q, causing ADCC activity and CDC activity, which are effector functions of the antibody, to be attenuated. This mutation can be used to any of the bispecific antibodies of the present disclosure having a particular structure.

The bispecific antibody in one embodiment includes (a) the first heavy chain that includes the variable region including the amino acid sequence set forth in SEQ ID NO: 238; (b) the second heavy chain that includes the variable region including the amino acid sequence set forth in SEQ ID NO: 238; (c) the light chain that includes the variable region including the amino acid sequence set forth in SEQ ID NO: 239; and (d) the scFv consisting of the amino acid sequence set forth in SEQ ID NO: 33 or 45, wherein in the constant region of the first heavy chain and the constant region of the second heavy chain, an asparagine residue at position 297 according to EU numbering has been substituted with a glycine residue (N297G) in the human IgG1 heavy chain constant region. In this embodiment, the bispecific antibody is efficiently delivered to the brain to reduce Aβ (oligomer Aβ, Aβ protofibril, and insoluble Aβ), and at the same time, to lower the risk of side effects of reducing reticulocytes (RET) due to the attenuation of ADCC activity and CDC activity. Further, because of the decrease in binding activity to C1q, lowering the risk of ARIA is also expected. The bispecific antibody in this embodiment has been improved compared to the bispecific antibody including L234A/L235A (cisLALA) mutations, because the N297G mutation is related to a greater decrease in effector functions due to the decrease in binding to the Fcγ receptor and C1q. For example, the bispecific antibody in this embodiment can bind to the Fcγ receptors, which are FcγRI, FcγRIIA, and FcγRIIIA, at KD values of 1.0×10⁻⁷ M or more (i.e., lower affinity), and in particular, the bispecific antibody in this embodiment does not substantially bind to FcγRIIA and FcγRIIIA. The bispecific antibody of this embodiment exhibits reduced ADCC activity and CDC activity due to a decrease in Fc effector functions, whereas its phagocytic activity against Aβ is maintained. Therefore, the bispecific antibody of this embodiment possesses both, Fc-mediated phagocytic activity (efficacy) necessary for clinical effect, and an improved toxicity profile (safety).

The bispecific antibody in one embodiment includes (a) the first heavy chain that includes the variable region including the amino acid sequence set forth in SEQ ID NO: 238; (b) the second heavy chain that includes the variable region including the amino acid sequence set forth in SEQ ID NO: 238; (c) the light chain that includes the variable region including the amino acid sequence set forth in SEQ ID NO: 239; and (d) the dsscFv consisting of the amino acid sequence set forth in SEQ ID NO: 263 or 271, wherein in the constant region of the first heavy chain and the constant region of the second heavy chain, an asparagine residue at position 297 according to EU numbering has been substituted with a glycine residue (N297G) in the human IgG1 heavy chain constant region. In this embodiment, the bispecific antibody is efficiently delivered to the brain to reduce Aβ (oligomer Aβ, Aβ protofibril, and insoluble Aβ), and at the same time, to lower the risk of side effects of reducing reticulocytes (RET) due to the attenuation of ADCC activity and CDC activity. The bispecific antibody of this embodiment has a lowered risk of ARIA, and because of the decrease in binding activity to C1q, a further reduction in the risk of ARIA is also expected. The bispecific antibody in this embodiment has been improved compared to the bispecific antibody including L234A/L235A (cisLALA) mutations, because the N297G mutation is related to a greater decrease in effector functions due to the decrease in binding to the Fcγ receptor and C1q. For example, the bispecific antibody in this embodiment can bind to the Fcγ receptors, which are FcγRI, FcγRIIA, and FcγRIIIA, at KD values of 1.0×10⁻⁷ M or more (i.e., lower affinity), and in particular, the bispecific antibody in this embodiment does not substantially bind to FcγRIIA and FcγRIIIA. The bispecific antibody of this embodiment exhibits reduced ADCC activity and CDC activity due to a decrease in Fc effector functions, whereas its phagocytic activity against Aβ is maintained. Therefore, the bispecific antibody of this embodiment possesses both, Fc-mediated phagocytic activity (efficacy) necessary for clinical effect, and an improved toxicity profile (safety).

### Hp

In the bispecific antibody according to one embodiment, the constant region of the first heavy chain of the first antigen-binding polypeptide includes K409D and K439E, and the constant region of the second heavy chain of the first antigen-binding polypeptide includes D356K and D399K. The bispecific antibody including the first antigen-binding polypeptide including these mutation of the amino acid residue in the heavy chain constant region, and the second antigen-binding polypeptide including the heavy chain variable region including HCDR1 consisting of the amino acid sequence set forth in SEQ ID NO: 2, HCDR2 consisting of the amino acid sequence set forth in SEQ ID NO: 8 or SEQ ID NO: 13, and HCDR3 consisting of the amino acid sequence set forth in SEQ ID NO: 3, and a light chain variable region including LCDR1 consisting of the amino acid sequence set forth in SEQ ID NO: 4, LCDR2 consisting of the amino acid sequence set forth in SEQ ID NO: 5, and LCDR3 consisting of the amino acid sequence set forth in SEQ ID NO: 6, exhibits a high level of antibody production, good separation capability, and excellent acid resistance. These mutations can be used in any of the bispecific antibodies of the present disclosure having the particular structure. The term “good separation capability” used herein refers to, in a purification step (for example, chromatography), satisfactory separation from impurities is achieved. More specifically, a bispecific antibody of the present disclosure has “good separation capability” when the percentage of protein of interest (POI) in purified product is greater than 90%, or not particularly limited, but preferably greater than 95%, as determined by non-reduced SDS-PAGE analysis of purified product. In a particular embodiment, purification step is performed by a combination of affinity chromatography (Protein A column) and cation exchange chromatography, and then purified product is analyzed by non-reduced SDS-PAGE to evaluate separation capability.

The bispecific antibody in one embodiment includes (a) the first heavy chain that includes the variable region including the amino acid sequence set forth in SEQ ID NO: 238; (b) the second heavy chain that includes the variable region including the amino acid sequence set forth in SEQ ID NO: 238; (c) the light chain that includes the variable region including the amino acid sequence set forth in SEQ ID NO: 239; and (d) the scFv consisting of the amino acid sequence set forth in SEQ ID NO: 33 or 45, wherein the constant region of the first heavy chain includes K409D and K439E, and the constant region of the second heavy chain includes D356K and D399K. In this embodiment, a high amount of antibody production, good separation capability, and excellent acid resistance of the bispecific antibody are provided. Therefore, the bispecific antibody of this embodiment has excellent manufacturing properties.

The bispecific antibody in one embodiment includes (a) the first heavy chain that includes the variable region including the amino acid sequence set forth in SEQ ID NO: 238; (b) the second heavy chain that includes the variable region including the amino acid sequence set forth in SEQ ID NO: 238; (c) the light chain that includes the variable region including the amino acid sequence set forth in SEQ ID NO: 239; and (d) the dsscFv consisting of the amino acid sequence set forth in SEQ ID NO: 263, wherein the constant region of the first heavy chain includes K409D and K439E, and the constant region of the second heavy chain includes D356K and D399K. In this embodiment, good separation capability, and excellent acid resistance of the bispecific antibody are provided. Therefore, the bispecific antibody of this embodiment has excellent manufacturing properties.

The bispecific antibody in one embodiment includes (a) the first heavy chain that includes the variable region including the amino acid sequence set forth in SEQ ID NO: 238; (b) the second heavy chain that includes the variable region including the amino acid sequence set forth in SEQ ID NO: 238; (c) the light chain that includes the variable region including the amino acid sequence set forth in SEQ ID NO: 239; and (d) the dsscFv consisting of the amino acid sequence set forth in SEQ ID NO: 271, wherein the constant region of the first heavy chain includes K409D and K439E, and the constant region of the second heavy chain includes D356K and D399K. In this embodiment, a high amount of antibody production, good separation capability, and excellent acid resistance of the bispecific antibody are provided. Therefore, the bispecific antibody of this embodiment has excellent manufacturing properties.

### HΔK

In the bispecific antibody according to one embodiment, the lysine residue at the C-terminus of the first heavy chain of the first antigen-binding polypeptide is deleted (HΔK). The bispecific antibody including the first antigen-binding polypeptide including the deletion of the amino acid residue in the heavy chain constant region, and the second antigen-binding polypeptide including the heavy chain variable region including HCDR1 consisting of the amino acid sequence set forth in SEQ ID NO: 2, HCDR2 consisting of the amino acid sequence set forth in SEQ ID NO: 8 or SEQ ID NO: 13, and HCDR3 consisting of the amino acid sequence set forth in SEQ ID NO: 3, and a light chain variable region including LCDR1 consisting of the amino acid sequence set forth in SEQ ID NO: 4, LCDR2 consisting of the amino acid sequence set forth in SEQ ID NO: 5, and LCDR3 consisting of the amino acid sequence set forth in SEQ ID NO: 6, exhibits an increased antibody production. This mutation can be used with any of the bispecific antibodies of the present disclosure having the particular structure.

The bispecific antibody in one embodiment includes (a) the first heavy chain that includes the variable region including the amino acid sequence set forth in SEQ ID NO: 238; (b) the second heavy chain that includes the variable region including the amino acid sequence set forth in SEQ ID NO: 238; (c) the light chain that includes the variable region including the amino acid sequence set forth in SEQ ID NO: 239; and (d) the scFv consisting of the amino acid sequence set forth in SEQ ID NO: 33 or 45, wherein the lysine residue at the C-terminus of the first heavy chain is deleted (HΔK). In this embodiment, a high amount of antibody production of the bispecific antibody is provided. Therefore, the bispecific antibody of this embodiment has excellent manufacturing properties.

The bispecific antibody in one embodiment includes (a) the first heavy chain that includes the variable region including the amino acid sequence set forth in SEQ ID NO: 238; (b) the second heavy chain that includes the variable region including the amino acid sequence set forth in SEQ ID NO: 238; (c) the light chain that includes the variable region including the amino acid sequence set forth in SEQ ID NO: 239; and (d) the dsscFv consisting of the amino acid sequence set forth in SEQ ID NO: 263, wherein the lysine residue at the C-terminus of the first heavy chain is deleted (HΔK).

The bispecific antibody in one embodiment includes (a) the first heavy chain that includes the variable region including the amino acid sequence set forth in SEQ ID NO: 238; (b) the second heavy chain that includes the variable region including the amino acid sequence set forth in SEQ ID NO: 238; (c) the light chain that includes the variable region including the amino acid sequence set forth in SEQ ID NO: 239; and (d) the dsscFv consisting of the amino acid sequence set forth in SEQ ID NO: 271, wherein the lysine residue at the C-terminus of the first heavy chain is deleted (HΔK). In this embodiment, a high amount of antibody production of the bispecific antibody is provided. Therefore, the bispecific antibody of this embodiment has excellent manufacturing properties.

### N297G and Hp

In the bispecific antibody according to one embodiment, the constant region of the first heavy chain of the first antigen-binding polypeptide includes N297G, K409D, and K439E, and the constant region of the second heavy chain of the first antigen-binding polypeptide includes N297G, D356K, and D399K. The bispecific antibody including the first antigen-binding polypeptide including these substitutions of the amino acid residue in the heavy chain constant region, and the second antigen-binding polypeptide including the heavy chain variable region including HCDR1 consisting of the amino acid sequence set forth in SEQ ID NO: 2, HCDR2 consisting of the amino acid sequence set forth in SEQ ID NO: 8 or SEQ ID NO: 13, and HCDR3 consisting of the amino acid sequence set forth in SEQ ID NO: 3, and a light chain variable region including LCDR1 consisting of the amino acid sequence set forth in SEQ ID NO: 4, LCDR2 consisting of the amino acid sequence set forth in SEQ ID NO: 5, and LCDR3 consisting of the amino acid sequence set forth in SEQ ID NO: 6, has a decreased binding activity to a Fcγ receptor and C1q, causing the attenuation of ADCC activity and CDC activity, which are effector functions of the antibody, and at the same time, exhibits a high amount of antibody production, good separation capability, and excellent acid resistance. These mutations can be used in any of the bispecific antibodies of the present disclosure having the particular structure.

The bispecific antibody in one embodiment includes (a) the first heavy chain that includes the variable region including the amino acid sequence set forth in SEQ ID NO: 238; (b) the second heavy chain that includes the variable region including the amino acid sequence set forth in SEQ ID NO: 238; (c) the light chain that includes the variable region including the amino acid sequence set forth in SEQ ID NO: 239; and (d) the scFv consisting of the amino acid sequence set forth in SEQ ID NO: 33 or 45, wherein the constant region of the first heavy chain includes N297G, K409D, and K439E, and the constant region of the second heavy chain includes N297G, D356K and D399K. In this embodiment, the bispecific antibody is efficiently delivered to the brain to reduce Aβ (oligomer Aβ, Aβ protofibril, and insoluble Aβ), and at the same time, to lower the risk of side effects of reducing reticulocytes (RET) due to the attenuation of ADCC activity and CDC activity. Further, because of the decrease in binding activity to C1q, lowering the risk of ARIA is also expected. In addition, a high amount of antibody production, good separation capability, and excellent acid resistance of the bispecific antibody are provided. The bispecific antibody in this embodiment has been improved compared to the bispecific antibody including L234A/L235A (cisLALA) mutations, because the N297G mutation is related to a greater decrease in effector functions due to the decrease in binding to the Fcγ receptor and C1q. For example, the bispecific antibody in this embodiment can bind to the Fcγ receptors, which are FcγRI, FcγRIIA, and FcγRIIIA, at KD values of 1.0×10⁻⁷ M or more (i.e., lower affinity), and in particular, the bispecific antibody in this embodiment does not substantially bind to FcγRIIA and FcγRIIIA. The bispecific antibody of this embodiment exhibits reduced ADCC activity and CDC activity due to a decrease in Fc effector functions, whereas its phagocytic activity against Aβ is maintained. Therefore, through the combination of these features, the bispecific antibody of this embodiment possesses excellent manufacturing properties, Fc-mediated phagocytic activity (efficacy) necessary for clinical effect, and an improved toxicity profile (safety).

The bispecific antibody in one embodiment includes (a) the first heavy chain that includes the variable region including the amino acid sequence set forth in SEQ ID NO: 238; (b) the second heavy chain that includes the variable region including the amino acid sequence set forth in SEQ ID NO: 238; (c) the light chain that includes the variable region including the amino acid sequence set forth in SEQ ID NO: 239; and (d) the dsscFv consisting of the amino acid sequence set forth in SEQ ID NO: 263, wherein the constant region of the first heavy chain includes N297G, K409D, and K439E, and the constant region of the second heavy chain includes N297G, D356K and D399K. In this embodiment, the bispecific antibody is efficiently delivered to the brain to reduce Aβ (oligomer Aβ, Aβ protofibril, and insoluble Aβ), and at the same time, to lower the risk of side effects of reducing reticulocytes (RET) due to the attenuation of ADCC activity and CDC activity. The bispecific antibody of this embodiment has a lowered risk of ARIA, and because of the decrease in binding activity to C1q, a further reduction in the risk of ARIA is also expected. In addition, good separation capability, and excellent acid resistance of the bispecific antibody are provided. The bispecific antibody in this embodiment has been improved compared to the bispecific antibody including L234A/L235A (cisLALA) mutations, because the N297G mutation is related to a greater decrease in effector functions due to the decrease in binding to the Fcγ receptor and C1q. For example, the bispecific antibody in this embodiment can bind to the Fcγ receptors, which are FcγRI, FcγRIIA, and FcγRIIIA, at KD values of 1.0×10⁻⁷ M or more (i.e., lower affinity), and in particular, the bispecific antibody in this embodiment does not substantially bind to FcγRIIA and FcγRIIIA. The bispecific antibody of this embodiment exhibits reduced ADCC activity and CDC activity due to a decrease in Fc effector functions, whereas its phagocytic activity against Aβ is maintained. Therefore, through the combination of these features, the bispecific antibody of this embodiment possesses excellent manufacturing properties, Fc-mediated phagocytic activity (efficacy) necessary for clinical effect, and an improved toxicity profile (safety).

The bispecific antibody in one embodiment includes (a) the first heavy chain that includes the variable region including the amino acid sequence set forth in SEQ ID NO: 238; (b) the second heavy chain that includes the variable region including the amino acid sequence set forth in SEQ ID NO: 238; (c) the light chain that includes the variable region including the amino acid sequence set forth in SEQ ID NO: 239; and (d) the dsscFv consisting of the amino acid sequence set forth in SEQ ID NO: 271, wherein the constant region of the first heavy chain includes N297G, K409D, and K439E, and the constant region of the second heavy chain includes N297G, D356K and D399K. In this embodiment, the bispecific antibody is efficiently delivered to the brain to reduce Aβ (oligomer Aβ, Aβ protofibril, and insoluble Aβ), and at the same time, to lower the risk of side effects of reducing reticulocytes (RET) due to the attenuation of ADCC activity and CDC activity. The bispecific antibody of this embodiment has a lowered risk of ARIA, and because of the decrease in binding activity to C1q, a further reduction in the risk of ARIA is also expected. In addition, a high amount of antibody production, good separation capability, and excellent acid resistance of the bispecific antibody are provided. The bispecific antibody in this embodiment has been improved compared to the bispecific antibody including L234A/L235A (cisLALA) mutations, because the N297G mutation is related to a greater decrease in effector functions due to the decrease in binding to the Fcγ receptor and C1q. For example, the bispecific antibody in this embodiment can bind to the Fcγ receptors, which are FcγRI, FcγRIIA, and FcγRIIIA, at KD values of 1.0×10⁻⁷ M or more (i.e., lower affinity), and in particular, the bispecific antibody in this embodiment does not substantially bind to FcγRIIA and FcγRIIIA. The bispecific antibody of this embodiment exhibits reduced ADCC activity and CDC activity due to a decrease in Fc effector functions, whereas its phagocytic activity against Aβ is maintained. Therefore, through the combination of these features, the bispecific antibody of this embodiment possesses excellent manufacturing properties, Fc-mediated phagocytic activity (efficacy) necessary for clinical effect, and an improved toxicity profile (safety).

### N297G and HΔK

In the bispecific antibody according to one embodiment, the constant region of the first heavy chain and the constant region of the second heavy chain of the first antigen-binding polypeptide each include N297G, and the lysine residue at the C-terminus of the first heavy chain of the first antigen-binding polypeptide is deleted (HΔK). The bispecific antibody including the first antigen-binding polypeptide including these mutation of the amino acid residue in the heavy chain constant region, and the second antigen-binding polypeptide including the heavy chain variable region including HCDR1 consisting of the amino acid sequence set forth in SEQ ID NO: 2, HCDR2 consisting of the amino acid sequence set forth in SEQ ID NO: 8 or SEQ ID NO: 13, and HCDR3 consisting of the amino acid sequence set forth in SEQ ID NO: 3, and a light chain variable region including LCDR1 consisting of the amino acid sequence set forth in SEQ ID NO: 4, LCDR2 consisting of the amino acid sequence set forth in SEQ ID NO: 5, and LCDR3 consisting of the amino acid sequence set forth in SEQ ID NO: 6, has a decreased binding activity to a Fcγ receptor and C1q, causing the attenuation of ADCC activity and CDC activity, which are effector functions of the antibody, and at the same time, exhibits an increased amount of antibody production. These mutations can be used in any of the bispecific antibodies of the present disclosure having the particular structure.

The bispecific antibody in one embodiment includes (a) the first heavy chain that includes the variable region including the amino acid sequence set forth in SEQ ID NO: 238; (b) the second heavy chain that includes the variable region including the amino acid sequence set forth in SEQ ID NO: 238; (c) the light chain that includes the variable region including the amino acid sequence set forth in SEQ ID NO: 239; and (d) the scFv consisting of the amino acid sequence set forth in SEQ ID NO: 33 or 45, wherein the constant region of the first heavy chain and the constant region of the second heavy chain each include N297G, and the lysine residue at the C-terminus of the first heavy chain is deleted (HΔK). In this embodiment, the bispecific antibody is efficiently delivered to the brain to reduce Aβ (oligomer Aβ, Aβ protofibril, and insoluble Aβ), and at the same time, to lower the risk of side effects of reducing reticulocytes (RET) due to the attenuation of ADCC activity and CDC activity. Further, because of the decrease in binding activity to C1q, lowering the risk of ARIA is also expected. In addition, a high amount of antibody production of the bispecific antibody is provided. The bispecific antibody in this embodiment has been improved compared to the bispecific antibody including L234A/L235A (cisLALA) mutations, because the N297G mutation is related to a greater decrease in effector functions due to the decrease in binding to the Fcγ receptor and C1q. For example, the bispecific antibody in this embodiment can bind to the Fcγ receptors, which are FcγRI, FcγRILA, and FcγRIIIA, at KD values of 1.0×10⁻⁷ M or more (i.e., lower affinity), and in particular, the bispecific antibody in this embodiment does not substantially bind to FcγRIIA and FcγRIIIA. The bispecific antibody of this embodiment exhibits reduced ADCC activity and CDC activity due to a decrease in Fc effector functions, whereas its phagocytic activity against Aβ is maintained. Therefore, through the combination of these features, the bispecific antibody of this embodiment possesses excellent manufacturing properties, Fc-mediated phagocytic activity (efficacy) necessary for clinical effect, and an improved toxicity profile (safety).

The bispecific antibody in one embodiment includes (a) the first heavy chain that includes the variable region including the amino acid sequence set forth in SEQ ID NO: 238; (b) the second heavy chain that includes the variable region including the amino acid sequence set forth in SEQ ID NO: 238; (c) the light chain that includes the variable region including the amino acid sequence set forth in SEQ ID NO: 239; and (d) the dsscFv consisting of the amino acid sequence set forth in SEQ ID NO: 263, wherein the constant region of the first heavy chain and the constant region of the second heavy chain each include N297G, and the lysine residue at the C-terminus of the first heavy chain is deleted (HΔK). In this embodiment, the bispecific antibody is efficiently delivered to the brain to reduce Aβ (oligomer Aβ, Aβ protofibril, and insoluble Aβ), and at the same time, to lower the risk of side effects of reducing reticulocytes (RET) due to the attenuation of ADCC activity and CDC activity. The bispecific antibody of this embodiment has a lowered risk of ARIA, and because of the decrease in binding activity to C1q, a further reduction in the risk of ARIA is also expected. The bispecific antibody in this embodiment has been improved compared to the bispecific antibody including L234A/L235A (cisLALA) mutations, because the N297G mutation is related to a greater decrease in effector functions due to the decrease in binding to the Fcγ receptor and C1q. For example, the bispecific antibody in this embodiment can bind to the Fcγ receptors, which are FcγRI, FcγRIIA, and FcγRIIIA, at KD values of 1.0×10⁻⁷ M or more (i.e., lower affinity), and in particular, the bispecific antibody in this embodiment does not substantially bind to FcγRIIA and FcγRIIIA. The bispecific antibody of this embodiment exhibits reduced ADCC activity and CDC activity due to a decrease in Fc effector functions, whereas its phagocytic activity against Aβ is maintained. Therefore, through the combination of these features, the bispecific antibody of this embodiment possesses both, Fc-mediated phagocytic activity (efficacy) necessary for clinical effect, and an improved toxicity profile (safety).

The bispecific antibody in one embodiment includes (a) the first heavy chain that includes the variable region including the amino acid sequence set forth in SEQ ID NO: 238; (b) the second heavy chain that includes the variable region including the amino acid sequence set forth in SEQ ID NO: 238; (c) the light chain that includes the variable region including the amino acid sequence set forth in SEQ ID NO: 239; and (d) the dsscFv consisting of the amino acid sequence set forth in SEQ ID NO: 271, wherein the constant region of the first heavy chain and the constant region of the second heavy chain each include N297G, and the lysine residue at the C-terminus of the first heavy chain is deleted (HΔK). In this embodiment, the bispecific antibody is efficiently delivered to the brain to reduce Aβ (oligomer Aβ, Aβ protofibril, and insoluble Aβ), and at the same time, to lower the risk of side effects of reducing reticulocytes (RET) due to the attenuation of ADCC activity and CDC activity. The bispecific antibody of this embodiment has a lowered risk of ARIA, and because of the decrease in binding activity to C1q, a further reduction in the risk of ARIA is also expected. In addition, a high amount of antibody production of the bispecific antibody is provided. The bispecific antibody in this embodiment has been improved compared to the bispecific antibody including L234A/L235A (cisLALA) mutations, because the N297G mutation is related to a greater decrease in effector functions due to the decrease in binding to the Fcγ receptor and C1q. For example, the bispecific antibody in this embodiment can bind to the Fcγ receptors, which are FcγRI, FcγRIIA, and FcγRIIIA, at KD values of 1.0×10⁻⁷ M or more (i.e., lower affinity), and in particular, the bispecific antibody in this embodiment does not substantially bind to FcγRIIA and FcγRIIIA. The bispecific antibody of this embodiment exhibits reduced ADCC activity and CDC activity due to a decrease in Fc effector functions, whereas its phagocytic activity against Aβ is maintained. Therefore, through the combination of these features, the bispecific antibody of this embodiment possesses excellent manufacturing properties, Fc-mediated phagocytic activity (efficacy) necessary for clinical effect, and an improved toxicity profile (safety).

### Hp and HΔK

In the bispecific antibody according to one embodiment, the constant region of the first heavy chain of the first antigen-binding polypeptide includes K409D and K439E, the constant region of the second heavy chain of the first antigen-binding polypeptide includes D356K and D399K, and further, the lysine residue at the C-terminus of the first heavy chain of the first antigen-binding polypeptide is deleted (HΔK). The bispecific antibody including the first antigen-binding polypeptide including these mutation of the amino acid residue in the heavy chain constant region, and the second antigen-binding polypeptide including the heavy chain variable region including HCDR1 consisting of the amino acid sequence set forth in SEQ ID NO: 2, HCDR2 consisting of the amino acid sequence set forth in SEQ ID NO: 8 or SEQ ID NO: 13, and HCDR3 consisting of the amino acid sequence set forth in SEQ ID NO: 3, and a light chain variable region including LCDR1 consisting of the amino acid sequence set forth in SEQ ID NO: 4, LCDR2 consisting of the amino acid sequence set forth in SEQ ID NO: 5, and LCDR3 consisting of the amino acid sequence set forth in SEQ ID NO: 6, exhibits a high level of antibody production, good separation capability, and excellent acid resistance. These mutations can be used in any of the bispecific antibodies of the present disclosure having the particular structure.

The bispecific antibody in one embodiment includes (a) the first heavy chain that includes the variable region including the amino acid sequence set forth in SEQ ID NO: 238; (b) the second heavy chain that includes the variable region including the amino acid sequence set forth in SEQ ID NO: 238; (c) the light chain that includes the variable region including the amino acid sequence set forth in SEQ ID NO: 239; and (d) the scFv consisting of the amino acid sequence set forth in SEQ ID NO: 33 or 45, wherein the constant region of the first heavy chain includes K409D and K439E and the constant region of the second heavy chain includes D356K and D399K, and further, the lysine residue at the C-terminus of the first heavy chain is deleted (HΔK). In this embodiment, the bispecific antibody is efficiently delivered to the brain to reduce Aβ (oligomer Aβ, Aβ protofibril, and insoluble Aβ), and at the same time, a further higher amount of antibody production, good separation capability, and excellent acid resistance of the bispecific antibody are provided. Therefore, through the combination of these features, the bispecific antibody of this embodiment possesses further excellent manufacturing properties.

The bispecific antibody in one embodiment includes (a) the first heavy chain that includes the variable region including the amino acid sequence set forth in SEQ ID NO: 238; (b) the second heavy chain that includes the variable region including the amino acid sequence set forth in SEQ ID NO: 238; (c) the light chain that includes the variable region including the amino acid sequence set forth in SEQ ID NO: 239; and (d) the dsscFv consisting of the amino acid sequence set forth in SEQ ID NO: 263, wherein the constant region of the first heavy chain includes K409D and K439E and the constant region of the second heavy chain includes D356K and D399K, and further, the lysine residue at the C-terminus of the first heavy chain is deleted (HΔK). In this embodiment, the bispecific antibody is efficiently delivered to the brain to reduce Aβ (oligomer Aβ, Aβ protofibril, and insoluble Aβ), and at the same time, good separation capability, and excellent acid resistance of the bispecific antibody are provided. Therefore, through the combination of these features, the bispecific antibody of this embodiment possesses excellent manufacturing properties.

The bispecific antibody in one embodiment includes (a) the first heavy chain that includes the variable region including the amino acid sequence set forth in SEQ ID NO: 238; (b) the second heavy chain that includes the variable region including the amino acid sequence set forth in SEQ ID NO: 238; (c) the light chain that includes the variable region including the amino acid sequence set forth in SEQ ID NO: 239; and (d) the dsscFv consisting of the amino acid sequence set forth in SEQ ID NO: 271, wherein the constant region of the first heavy chain includes K409D and K439E and the constant region of the second heavy chain includes D356K and D399K, and further, the lysine residue at the C-terminus of the first heavy chain is deleted (HΔK). In this embodiment, the bispecific antibody is efficiently delivered to the brain to reduce Aβ (oligomer Aβ, Aβ protofibril, and insoluble Aβ), and at the same time, a further higher amount of antibody production, good separation capability, and excellent acid resistance of the bispecific antibody are provided. Therefore, through the combination of these features, the bispecific antibody of this embodiment possesses further excellent manufacturing properties.

### N297G, Hp and HΔK

In the bispecific antibody according to one embodiment, the constant region of the first heavy chain of the first antigen-binding polypeptide includes N297G, K409D, and K439E, the constant region of the second heavy chain of the first antigen-binding polypeptide includes N297G, D356K, and D399K, and further, the lysine residue at the C-terminus of the first heavy chain of the first antigen-binding polypeptide is deleted (HΔK). The bispecific antibody including the first antigen-binding polypeptide including these substitutions of the amino acid residue in the heavy chain constant region, and the second antigen-binding polypeptide including the heavy chain variable region including HCDR1 consisting of the amino acid sequence set forth in SEQ ID NO: 2, HCDR2 consisting of the amino acid sequence set forth in SEQ ID NO: 8 or SEQ ID NO: 13, and HCDR3 consisting of the amino acid sequence set forth in SEQ ID NO: 3, and a light chain variable region including LCDR1 consisting of the amino acid sequence set forth in SEQ ID NO: 4, LCDR2 consisting of the amino acid sequence set forth in SEQ ID NO: 5, and LCDR3 consisting of the amino acid sequence set forth in SEQ ID NO: 6, has a decreased binding activity to a Fcγ receptor and C1q, causing the attenuation of ADCC activity and CDC activity, which are effector functions of the antibody, and at the same time, exhibit a high amount of antibody production, good separation capability, and excellent acid resistance. These mutations can be used in any of the bispecific antibodies of the present disclosure having the particular structure.

The bispecific antibody in one embodiment includes (a) the first heavy chain that includes the variable region including the amino acid sequence set forth in SEQ ID NO: 238; (b) the second heavy chain that includes the variable region including the amino acid sequence set forth in SEQ ID NO: 238; (c) the light chain that includes the variable region including the amino acid sequence set forth in SEQ ID NO: 239; and (d) the scFv consisting of the amino acid sequence set forth in SEQ ID NO: 33 or 45, wherein the constant region of the first heavy chain includes N297G, K409D, and K439E, the constant region of the second heavy chain includes N297G, D356K, and D399K, and further, the lysine residue at the C-terminus of the first heavy chain is deleted (HΔK). In this embodiment, the bispecific antibody is efficiently delivered to the brain to reduce Aβ (oligomer Aβ, Aβ protofibril, or insoluble Aβ), and at the same time, to lower the risk of side effects of reducing reticulocytes (RET) due to the attenuation of ADCC activity and CDC activity. Further, because of the decrease in binding activity to C1q, lowering the risk of ARIA is also expected. In addition, a further higher amount of antibody production, good separation capability, and excellent acid resistance of the bispecific antibody are provided. The bispecific antibody in this embodiment has been improved compared to the bispecific antibody including L234A/L235A (cisLALA) mutations, because the N297G mutation is related to a greater decrease in effector functions due to the decrease in binding to the Fcγ receptor and C1q. For example, the bispecific antibody in this embodiment can bind to the Fcγ receptors, which are FcγRI, FcγRIIA, and FcγRIIIA, at KD values of 1.0×10⁻⁷ M or more (i.e., lower affinity), and in particular, the bispecific antibody in this embodiment does not substantially bind to FcγRIIA and FcγRIIIA. The bispecific antibody of this embodiment exhibits reduced ADCC activity and CDC activity due to a decrease in Fc effector functions, whereas its phagocytic activity against Aβ is maintained. Therefore, through the combination of these features, the bispecific antibody of this embodiment possesses further excellent manufacturing properties, Fc-mediated phagocytic activity (efficacy) necessary for clinical effect, and an improved toxicity profile (safety).

The bispecific antibody in one embodiment includes (a) the first heavy chain that includes the variable region including the amino acid sequence set forth in SEQ ID NO: 238; (b) the second heavy chain that includes the variable region including the amino acid sequence set forth in SEQ ID NO: 238; (c) the light chain that includes the variable region including the amino acid sequence set forth in SEQ ID NO: 239; and (d) the dsscFv consisting of the amino acid sequence set forth in SEQ ID NO: 263, wherein the constant region of the first heavy chain includes N297G, K409D, and K439E, the constant region of the second heavy chain includes N297G, D356K, and D399K, and further, the lysine residue at the C-terminus of the first heavy chain is deleted (HΔK). In this embodiment, the bispecific antibody is efficiently delivered to the brain to reduce Aβ (oligomer Aβ, Aβ protofibril, or insoluble Aβ), and at the same time, to lower the risk of side effects of reducing reticulocytes (RET) due to the attenuation of ADCC activity and CDC activity. The bispecific antibody of this embodiment has a lowered risk of ARIA, and because of the decrease in binding activity to C1q, a further reduction in the risk of ARIA is also expected. In addition, good separation capability, and excellent acid resistance of the bispecific antibody are provided. The bispecific antibody in this embodiment has been improved compared to the bispecific antibody including L234A/L235A (cisLALA) mutations, because the N297G mutation is related to a greater decrease in effector functions due to the decrease in binding to the Fcγ receptor and C1q. For example, the bispecific antibody in this embodiment can bind to the Fcγ receptors, which are FcγRI, FcγRIIA, and FcγRIIIA, at KD values of 1.0×10⁻⁷ M or more (i.e., lower affinity), and in particular, the bispecific antibody in this embodiment does not substantially bind to FcγRIIA and FcγRIIIA. The bispecific antibody of this embodiment exhibits reduced ADCC activity and CDC activity due to a decrease in Fc effector functions, whereas its phagocytic activity against Aβ is maintained. Therefore, through the combination of these features, the bispecific antibody of this embodiment possesses excellent manufacturing properties, Fc-mediated phagocytic activity (efficacy) necessary for clinical effect, and an improved toxicity profile (safety).

The bispecific antibody in one embodiment includes (a) the first heavy chain that includes the variable region including the amino acid sequence set forth in SEQ ID NO: 238; (b) the second heavy chain that includes the variable region including the amino acid sequence set forth in SEQ ID NO: 238; (c) the light chain that includes the variable region including the amino acid sequence set forth in SEQ ID NO: 239; and (d) the dsscFv consisting of the amino acid sequence set forth in SEQ ID NO: 271, wherein the constant region of the first heavy chain includes N297G, K409D, and K439E, the constant region of the second heavy chain includes N297G, D356K, and D399K, and further, the lysine residue at the C-terminus of the first heavy chain is deleted (HΔK). In this embodiment, the bispecific antibody is efficiently delivered to the brain to reduce Aβ (oligomer Aβ, Aβ protofibril, or insoluble Aβ), and at the same time, to lower the risk of side effects of reducing reticulocytes (RET) due to the attenuation of ADCC activity and CDC activity. The bispecific antibody of this embodiment has a lowered risk of ARIA, and because of the decrease in binding activity to C1q, a further reduction in the risk of ARIA is also expected. In addition, a further higher amount of antibody production, good separation capability, and excellent acid resistance of the bispecific antibody are provided. Furthermore, the bispecific antibody of this embodiment exhibits suitable stability in solution. More specifically, the bispecific antibody of this embodiment exhibits suitable storage stability in solution at the storage conditions such as concentrations and durations within the target product profile specifications, in that high-molecular-weight (HMW) species are maintained within the desired specifications. For example, even when the bispecific antibodies of this embodiment are stored in solution for longer periods and/or at higher concentrations, the proportion of HMW species remains low. The bispecific antibody of this embodiment (comprising the dsscFv consisting of the amino acid sequence set forth in SEQ ID NO: 271) shows improved storage stability in solution as compared to the bispecific antibodies having the same structure, but comprising either the scFv consisting of the amino acid sequence set forth in SEQ ID NO: 33 or 45, or the dsscFv consisting of the amino acid sequence set forth in SEQ ID NO: 263. The bispecific antibody in this embodiment has been improved compared to the bispecific antibody including L234A/L235A (cisLALA) mutations, because the N297G mutation is related to a greater decrease in effector functions due to the decrease in binding to the Fcγ receptor and C1q. For example, the bispecific antibody in this embodiment can bind to the Fcγ receptors, which are FcγRI, FcγRIIA, and FcγRIIIA, at KD values of 1.0×10⁻⁷ M or more (i.e., lower affinity), and in particular, the bispecific antibody in this embodiment does not substantially bind to FcγRIIA and FcγRIIIA. The bispecific antibody of this embodiment exhibits reduced ADCC activity and CDC activity due to a decrease in Fc effector functions, whereas its phagocytic activity against Aβ is maintained. Therefore, through the combination of these features, the bispecific antibody of this embodiment possesses further excellent manufacturing properties, suitable stability, Fc-mediated phagocytic activity (efficacy) necessary for clinical effect, and an improved toxicity profile (safety).

In the bispecific antibody according to one embodiment, the first antigen-binding polypeptide binding to amyloid beta (Aβ) is the first antigen-binding polypeptide binding to amyloid beta (Aβ) protofibril.

Specific examples of the bispecific antibody as a fusion protein include BR478, BR479, BR586, BR802, BR803, BR838, and BR839 in Experimental Examples.

Examples of the bispecific antibody in one embodiment include a bispecific antibody including a polypeptide consisting of the amino acid sequence set forth in SEQ ID NO: 189, a polypeptide consisting of the amino acid sequence set forth in SEQ ID NO: 191, and a polypeptide consisting of the amino acid sequence set forth in SEQ ID NO: 197, or a bispecific antibody including a polypeptide consisting of the amino acid sequence set forth in SEQ ID NO: 189, a polypeptide consisting of the amino acid sequence set forth in SEQ ID NO: 193, and a polypeptide consisting of the amino acid sequence set forth in SEQ ID NO: 197.

Examples of the bispecific antibody in another embodiment include a bispecific antibody including a polypeptide consisting of the amino acid sequence set forth in SEQ ID NO: 189, a polypeptide consisting of the amino acid sequence set forth in SEQ ID NO: 269, and a polypeptide consisting of the amino acid sequence set forth in SEQ ID NO: 197, or a bispecific antibody including a polypeptide consisting of the amino acid sequence set forth in SEQ ID NO: 189, a polypeptide consisting of the amino acid sequence set forth in SEQ ID NO: 277, and a polypeptide consisting of the amino acid sequence set forth in SEQ ID NO: 197.

Further specific examples of the bispecific antibody as a fusion protein include BR834, BR835, BR836, and BR837 in Experimental Examples.

Examples of the bispecific antibody in one embodiment include a bispecific antibody including a polypeptide consisting of the amino acid sequence set forth in SEQ ID NO: 189, a polypeptide consisting of the amino acid sequence set forth in SEQ ID NO: 296, and a polypeptide consisting of the amino acid sequence set forth in SEQ ID NO: 197, or a bispecific antibody including a polypeptide consisting of the amino acid sequence set forth in SEQ ID NO: 189, a polypeptide consisting of the amino acid sequence set forth in SEQ ID NO: 298, and a polypeptide consisting of the amino acid sequence set forth in SEQ ID NO: 197.

Examples of the bispecific antibody in another embodiment include a bispecific antibody including a polypeptide consisting of the amino acid sequence set forth in SEQ ID NO: 189, a polypeptide consisting of the amino acid sequence set forth in SEQ ID NO: 300, and a polypeptide consisting of the amino acid sequence set forth in SEQ ID NO: 197, or a bispecific antibody including a polypeptide consisting of the amino acid sequence set forth in SEQ ID NO: 189, a polypeptide consisting of the amino acid sequence set forth in SEQ ID NO: 302, and a polypeptide consisting of the amino acid sequence set forth in SEQ ID NO: 197.

The bispecific antibody in one embodiment is a bispecific antibody including the first antigen-binding polypeptide that binds to amyloid beta (Aβ) and the second antigen-binding polypeptide that binds to a human transferrin receptor (TfR), in which the first antigen-binding polypeptide includes the first heavy chain, the second heavy chain, and two light chains.

The bispecific antibody in one embodiment is a bispecific antibody including the first antigen-binding polypeptide that binds to amyloid beta (Aβ) and the second antigen-binding polypeptide that binds to a human transferrin receptor (TfR), in which the first antigen-binding polypeptide includes the first heavy chain, the second heavy chain, and two light chains, and the second antigen-binding polypeptide is connected to the C-terminus of the constant region of the second heavy chain of the first antigen-binding polypeptide via a peptide linker.

Examples of the bispecific antibody in one embodiment include a bispecific antibody including the first antigen-binding polypeptide that binds to amyloid beta (Aβ) and the second antigen-binding polypeptide that binds to a human transferrin receptor (TfR), in which:
(i) the first antigen-binding polypeptide includes a first heavy chain consisting of the amino acid sequence set forth in SEQ ID NO: 238 and the amino acid sequence set forth in SEQ ID NO: 240, a second heavy chain consisting of the amino acid sequence set forth in SEQ ID NO: 238 and the amino acid sequence set forth in SEQ ID NO: 241, and a light chain consisting of the amino acid sequence set forth in SEQ ID NO: 239 and the amino acid sequence set forth in SEQ ID NO: 119;
(ii) the second antigen-binding polypeptide includes scFv consisting of the amino acid sequence set forth in SEQ ID NO: 33; and
(iii) the second antigen-binding polypeptide is connected to the C-terminus of the constant region of the second heavy chain of the first antigen-binding polypeptide, via a peptide linker consisting of the amino acid sequence set forth in SEQ ID NO: 79.

Examples of the bispecific antibody in another embodiment include a bispecific antibody including the first antigen-binding polypeptide that binds to amyloid beta (Aβ) and the second antigen-binding polypeptide that binds to a human transferrin receptor (TfR), in which:
(i) the first antigen-binding polypeptide includes a first heavy chain consisting of the amino acid sequence set forth in SEQ ID NO: 238 and the amino acid sequence set forth in SEQ ID NO: 240, a second heavy chain consisting of the amino acid sequence set forth in SEQ ID NO: 238 and the amino acid sequence set forth in SEQ ID NO: 241, and a light chain consisting of the amino acid sequence set forth in SEQ ID NO: 239 and the amino acid sequence set forth in SEQ ID NO: 119;
(ii) the second antigen-binding polypeptide includes scFv consisting of the amino acid sequence set forth in SEQ ID NO: 45; and
(iii) the second antigen-binding polypeptide is connected to the C-terminus of the constant region of the second heavy chain of the first antigen-binding polypeptide, via a peptide linker consisting of the amino acid sequence set forth in SEQ ID NO: 79.

Examples of the bispecific antibody in a particular embodiment include a bispecific antibody including the first antigen-binding polypeptide that binds to amyloid beta (Aβ) and the second antigen-binding polypeptide that binds to a human transferrin receptor (TfR), in which:
(i) the first antigen-binding polypeptide includes a first heavy chain consisting of the amino acid sequence set forth in SEQ ID NO: 238 and the amino acid sequence set forth in SEQ ID NO: 240, a second heavy chain consisting of the amino acid sequence set forth in SEQ ID NO: 238 and the amino acid sequence set forth in SEQ ID NO: 241, and a light chain consisting of the amino acid sequence set forth in SEQ ID NO: 239 and the amino acid sequence set forth in SEQ ID NO: 119;
(ii) the second antigen-binding polypeptide includes dsscFv consisting of the amino acid sequence set forth in SEQ ID NO: 263; and
(iii) the second antigen-binding polypeptide is connected to the C-terminus of the constant region of the second heavy chain of the first antigen-binding polypeptide, via a peptide linker consisting of the amino acid sequence set forth in SEQ ID NO: 79.

Examples of the bispecific antibody in a particular embodiment include a bispecific antibody including the first antigen-binding polypeptide that binds to amyloid beta (Aβ) and the second antigen-binding polypeptide that binds to a human transferrin receptor (TfR), in which:
(i) the first antigen-binding polypeptide includes a first heavy chain consisting of the amino acid sequence set forth in SEQ ID NO: 238 and the amino acid sequence set forth in SEQ ID NO: 240, a second heavy chain consisting of the amino acid sequence set forth in SEQ ID NO: 238 and the amino acid sequence set forth in SEQ ID NO: 241, and a light chain consisting of the amino acid sequence set forth in SEQ ID NO: 239 and the amino acid sequence set forth in SEQ ID NO: 119;
(ii) the second antigen-binding polypeptide includes dsscFv consisting of the amino acid sequence set forth in SEQ ID NO: 271; and
(iii) the second antigen-binding polypeptide is connected to the C-terminus of the constant region of the second heavy chain of the first antigen-binding polypeptide, via a peptide linker consisting of the amino acid sequence set forth in SEQ ID NO: 79.

Examples of the bispecific antibody in a particular embodiment include a bispecific antibody including the first antigen-binding polypeptide that binds to amyloid beta (Aβ) and the second antigen-binding polypeptide that binds to a human transferrin receptor (TfR), in which:
(i) the first antigen-binding polypeptide includes a first heavy chain consisting of the amino acid sequence set forth in SEQ ID NO: 238 and the amino acid sequence set forth in SEQ ID NO: 240, a second heavy chain consisting of the amino acid sequence set forth in SEQ ID NO: 238 and the amino acid sequence set forth in SEQ ID NO: 241, and a light chain consisting of the amino acid sequence set forth in SEQ ID NO: 239 and the amino acid sequence set forth in SEQ ID NO: 119;
(ii) the second antigen-binding polypeptide includes scFv consisting of the amino acid sequence set forth in SEQ ID NO: 289; and
(iii) the second antigen-binding polypeptide is connected to the C-terminus of the constant region of the second heavy chain of the first antigen-binding polypeptide, via a peptide linker consisting of the amino acid sequence set forth in SEQ ID NO: 79.

Examples of the bispecific antibody in a particular embodiment include a bispecific antibody including the first antigen-binding polypeptide that binds to amyloid beta (Aβ) and the second antigen-binding polypeptide that binds to a human transferrin receptor (TfR), in which:
(i) the first antigen-binding polypeptide includes a first heavy chain consisting of the amino acid sequence set forth in SEQ ID NO: 238 and the amino acid sequence set forth in SEQ ID NO: 240, a second heavy chain consisting of the amino acid sequence set forth in SEQ ID NO: 238 and the amino acid sequence set forth in SEQ ID NO: 241, and a light chain consisting of the amino acid sequence set forth in SEQ ID NO: 239 and the amino acid sequence set forth in SEQ ID NO: 119;
(ii) the second antigen-binding polypeptide includes scFv consisting of the amino acid sequence set forth in SEQ ID NO: 290; and
(iii) the second antigen-binding polypeptide is connected to the C-terminus of the constant region of the second heavy chain of the first antigen-binding polypeptide, via a peptide linker consisting of the amino acid sequence set forth in SEQ ID NO: 79.

Examples of the bispecific antibody in a particular embodiment include a bispecific antibody including the first antigen-binding polypeptide that binds to amyloid beta (Aβ) and the second antigen-binding polypeptide that binds to a human transferrin receptor (TfR), in which:
(i) the first antigen-binding polypeptide includes a first heavy chain consisting of the amino acid sequence set forth in SEQ ID NO: 238 and the amino acid sequence set forth in SEQ ID NO: 240, a second heavy chain consisting of the amino acid sequence set forth in SEQ ID NO: 238 and the amino acid sequence set forth in SEQ ID NO: 241, and a light chain consisting of the amino acid sequence set forth in SEQ ID NO: 239 and the amino acid sequence set forth in SEQ ID NO: 119;
(ii) the second antigen-binding polypeptide includes dsscFv consisting of the amino acid sequence set forth in SEQ ID NO: 291; and
(iii) the second antigen-binding polypeptide is connected to the C-terminus of the constant region of the second heavy chain of the first antigen-binding polypeptide, via a peptide linker consisting of the amino acid sequence set forth in SEQ ID NO: 79.

Examples of the bispecific antibody in a particular embodiment include a bispecific antibody including the first antigen-binding polypeptide that binds to amyloid beta (Aβ) and the second antigen-binding polypeptide that binds to a human transferrin receptor (TfR), in which:
(i) the first antigen-binding polypeptide includes a first heavy chain consisting of the amino acid sequence set forth in SEQ ID NO: 238 and the amino acid sequence set forth in SEQ ID NO: 240, a second heavy chain consisting of the amino acid sequence set forth in SEQ ID NO: 238 and the amino acid sequence set forth in SEQ ID NO: 241, and a light chain consisting of the amino acid sequence set forth in SEQ ID NO: 239 and the amino acid sequence set forth in SEQ ID NO: 119;
(ii) the second antigen-binding polypeptide includes dsscFv consisting of the amino acid sequence set forth in SEQ ID NO: 292; and
(iii) the second antigen-binding polypeptide is connected to the C-terminus of the constant region of the second heavy chain of the first antigen-binding polypeptide, via a peptide linker consisting of the amino acid sequence set forth in SEQ ID NO: 79.

The bispecific antibody of the present disclosure can cross the blood-brain barrier (BBB) due to the presence of the second antigen-binding polypeptide that binds to a human transferrin receptor (TfR) of the present disclosure (i.e., anti-TfR antibody or antigen-binding fragment thereof), and as a result, the first antigen-binding polypeptide that binds to amyloid beta (Aβ) not capable of crossing or having difficulty in crossing the blood-brain barrier (BBB) alone, is transferred into the brain by TfR when bound to the second antigen-binding polypeptide that binds to a human transferrin receptor (TfR) of the present disclosure, enabling it to show the bioactivity in the brain. On the other hand, the first antigen-binding polypeptide that binds to amyloid beta (Aβ) (i.e., anti-amyloid beta (Aβ) antibody or antigen-binding fragment thereof) can decrease the amount of amyloid in the brain, and as a result, it leads to treatment of an amyloid beta (Aβ)-related disease. Here, the “treatment” is to cure, suppress, or improve a disease or a symptom in an individual already suffered from or developed the disease or symptom.

### [Nucleic Acid, Vector, Host Cell, and Method for Producing Antibody]

One embodiment provides a nucleic acid (DNA) that encodes the bispecific antibody of the present disclosure. Examples of the nucleic acid that encodes the bispecific antibody including the polypeptide consisting of the amino acid sequence set forth in SEQ ID NO: 189, the polypeptide consisting of the amino acid sequence set forth in SEQ ID NO: 191, and the polypeptide consisting of the amino acid sequence set forth in SEQ ID NO: 197, include nucleic acids including nucleotide sequences set forth in SEQ ID NOs: 188, 190, and 196. In addition, examples of the nucleic acid that encodes the bispecific antibody including the polypeptide consisting of the amino acid sequence set forth in SEQ ID NO: 189, the polypeptide consisting of the amino acid sequence set forth in SEQ ID NO: 193, and the polypeptide consisting of the amino acid sequence set forth in SEQ ID NO: 197, include nucleic acids including nucleotide sequences set forth in SEQ ID NOs: 188, 192, and 196. Further, examples of the nucleic acid that encodes the bispecific antibody including the polypeptide consisting of the amino acid sequence set forth in SEQ ID NO: 189, a polypeptide consisting of the amino acid sequence set forth in SEQ ID NO: 269, and a polypeptide consisting of the amino acid sequence set forth in SEQ ID NO: 197, include nucleic acids including nucleotide sequences set forth in SEQ ID NOs: 188, 268, and 196, and examples of the nucleic acid that encodes the bispecific antibody including the polypeptide consisting of the amino acid sequence set forth in SEQ ID NO: 189, the polypeptide consisting of the amino acid sequence set forth in SEQ ID NO: 277, and the polypeptide consisting of the amino acid sequence set forth in SEQ ID NO: 197, include nucleic acids including nucleotide sequences set forth in SEQ ID NOs: 188, 276, and 196.

In one embodiment, the nucleic acid that encodes the bispecific antibody of the present disclosure refers to one or more nucleic acids including nucleotide sequences that encode each polypeptide constituting the bispecific antibody. For example, the nucleotide sequence that encodes the polypeptide of SEQ ID NO: 189 (SEQ ID NO: 188), the nucleotide sequence that encodes the polypeptide of SEQ ID NO: 191 (SEQ ID NO: 190), and the nucleotide sequence that encodes the polypeptide of SEQ ID NO: 197 (SEQ ID NO: 196) may be included in one nucleic acid, or may each be included in distinct nucleic acids. Similarly, for example, the nucleotide sequence that encodes the polypeptide of SEQ ID NO: 189 (SEQ ID NO: 188), the nucleotide sequence that encodes the polypeptide of SEQ ID NO: 193 (SEQ ID NO: 192), and the nucleotide sequence that encodes the polypeptide of SEQ ID NO: 197 (SEQ ID NO: 196) may be included in one nucleic acid, or may each be included in distinct nucleic acids. In addition, for example, the nucleotide sequence that encodes the polypeptide of SEQ ID NO: 189 (SEQ ID NO: 188), the nucleotide sequence that encodes the polypeptide of SEQ ID NO: 269 (SEQ ID NO: 268), and the nucleotide sequence that encodes the polypeptide of SEQ ID NO: 197 (SEQ ID NO: 196) may be included in one nucleic acid, or may each be included in distinct nucleic acids. Similarly, for example, the nucleotide sequence that encodes the polypeptide of SEQ ID NO: 189 (SEQ ID NO: 188), the nucleotide sequence that encodes the polypeptide of SEQ ID NO: 277 (SEQ ID NO: 276), and the nucleotide sequence that encodes the polypeptide of SEQ ID NO: 197 (SEQ ID NO: 196) may be included in one nucleic acid, or may each be included in distinct nucleic acids.

One embodiment provides a vector including a nucleic acid (DNA) that encodes the bispecific antibody of the present disclosure, and further provides a recombinant cell in which the vector has been introduced into a host cell. The vector and host cell are not particularly limited as long as they are those commonly used.

The expression vector can contain one or more additional sequences, including, but not limited to, regulatory sequences (e.g., promoter and enhancer), selection markers, and polyadenylation signals. Vectors that transform and/or transfect a wide variety of host cells are well known, and examples thereof include, but not limited to, plasmids, phagemids, cosmids, Baculoviruses, bacmids, bacterial artificial chromosome (BAC), yeast artificial chromosomes (YAC), and other bacterial vectors, yeast vectors, and viral vectors.

The host cell is not particularly limited, and a prokaryotic cell or a eukaryotic cell can be used. Examples of the eukaryotic cell include cells derived from yeast, plants, animals such as mammals, for example, rodents, or humans, and specific examples include cell lines of NS0, CHO, CHOK1, perC.6, Tk-ts13, BHK, HEK293 cells, COS-7, T98G, CV-1/EBNA, L cells, C127, 3T3, HeLa, NS1, and Sp2/0 myeloma cells.

The bispecific antibody of the present disclosure can be produced by a method known to those skilled in the art. For example, it may be produced by a known peptide synthesis method (e.g., solid phase synthesis), or artificial synthesis, and can also be obtained by expressing it in the above recombinant cells to collect and purify. In addition, for easier purification or easier detection by assay, it can be produced as a fusion protein with a tag (e.g., His tag) or a detectable marker protein (e.g., GFP).

The method for producing the bispecific antibody of the present disclosure includes a step of culturing the above-described host cell to produce a bispecific antibody. Specifically, a bispecific antibody, which is a fusion protein, can be obtained by producing a nucleic acid that encodes the fusion protein in which the second antigen-binding polypeptide, which is an anti-TfR antibody or an antigen-binding fragment thereof, and the first antigen-binding polypeptide, which is an anti-amyloid beta (Aβ) antibody or an antigen-binding fragment thereof functionally linked, via a peptide linker in some cases, introducing the nucleic acid functionally linked with an expression promoter into an appropriate expression vector, and producing in the host cell.

[Pharmaceutical Composition, Use, and Method] The pharmaceutical composition of the present disclosure includes the bispecific antibody of the present disclosure. The pharmaceutical composition in one embodiment includes an effective amount of the bispecific antibody of the present disclosure.

The pharmaceutical composition of the present disclosure can be produced according to known methods such as the methods, for example, described in the Pharmacopoeia of Japan (JP), the United States Pharmacopeia (USP), or the European Pharmacopoeia (EP).

The pharmaceutical composition in the present embodiment can include a pharmaceutically acceptable carrier.

The number of doses and dose intervals of the pharmaceutical composition in this embodiment are not particularly limited, and it can be administered as a single dose, or it can be administered as multiple doses with intervals of several days to several months. The administration route of the pharmaceutical composition in this embodiment can be appropriately designed by those skilled in the art.

The pharmaceutical composition in one embodiment is a pharmaceutical composition including an effective amount of the bispecific antibody of the present disclosure and for treating an amyloid beta (Aβ)-related disease. The therapeutic effects of the bispecific antibody of the present disclosure depend on the therapeutic effects of the first antigen-binding polypeptide included in the bispecific antibody. By administering the pharmaceutical composition of this embodiment to a subject, the bispecific antibody is transferred into the brain in the subject by the second antigen-binding polypeptide, and an amyloid beta (Aβ)-related disease can be treated in the brain by the bioactivity of the first antigen-binding polypeptide.

Use in one embodiment is use in transport of the first antigen-binding polypeptide in the bispecific antibody of the present disclosure to the brain. The method in one embodiment is a method for transporting the first antigen-binding polypeptide to the brain by using the second antigen-binding polypeptide in the bispecific antibody of the present disclosure.

Use in one embodiment is use of the bispecific antibody of the present disclosure in the manufacture of a medicament for the treatment of an amyloid beta (Aβ)-related disease. The method in one embodiment is a method of treating an amyloid beta (Aβ)-related disease, including administering an effective amount of the bispecific antibody of the present disclosure to a subject in need thereof. The bispecific antibody in one embodiment is a bispecific antibody for use in the treatment of an amyloid beta (Aβ)-related disease. Use in another embodiment is use of the bispecific antibody of the present disclosure for the treatment an amyloid beta (Aβ)-related disease.

In one embodiment, the amyloid beta (Aβ)-related disease is Alzheimer's disease (AD), mild cognitive impairment due to AD (MCI due to AD), preclinical AD, or Down's syndrome.

In another embodiment, the amyloid beta (Aβ)-related disease is Alzheimer's disease (AD), mild cognitive impairment due to AD (MCI due to AD), or preclinical AD.

In one embodiment, the amyloid beta (Aβ)-related disease is Alzheimer's disease (AD). In another embodiment, the amyloid beta (Aβ)-related disease is mild cognitive impairment due to AD (MCI due to AD). In a further embodiment, the amyloid beta (Aβ)-related disease is preclinical AD. In a further embodiment, the amyloid beta (Aβ)-related disease is Down's syndrome.

The present invention will be described in more detail using Examples described below; however, the present invention is not limited to these.

### EXAMPLES

### Experimental Example 1: Production of Bispecific Anti-Human TfR/EphA4 Antibodies

### (1) Construction of Expression Vector for Soluble TfR Protein Genes

To obtain antibodies that bind to human TfR and monkey TfR, proteins in which a polyhistidine/maltose-binding protein tag (His-MBP tag) is fused to the N-terminus of the soluble human TfR sequence (GenBank Accession No. NP_001121620.1, positions 101-760) and the monkey TfR sequence corresponding to the soluble human TfR region (GenBank Accession No. XP_045243212, positions 101-760) (hereinafter referred to as “His-MBP-human TfR protein” and “His-MBP-monkey TfR protein,” corresponding to SEQ ID NOs: 16 and 17) were prepared according to the procedures described below.

First, the DNA sequence that encodes the amino acid sequence of a human TfR extracellular region (positions 89-760) was amplified by PCR using human-derived total RNA, the amplified fragment was cloned into the C-terminal end of the tag of a pcDNA3.4 vector containing a His-MBP tag (Thermo Fisher Scientific, hereinafter “Thermo”), and thereby a pcDNA3.4-His-MBP-human TfR extracellular region gene expression vector was constructed. A DNA sequence that encodes the soluble human TfR amino acid sequence was amplified by PCR using the pcDNA3.4-His-MBP-human TfR extracellular region expression vector as a template. A DNA sequence that encodes the soluble monkey TfR amino acid sequence was de novo synthesized by GenScript. These DNA sequences that encode the soluble TfR amino acid sequences were cloned into the C-terminal end of the tag of the pcDNA3.4 vector containing a His-MBP tag, and thereby a pcDNA3.4-His-MBP-human TfR gene expression vector and a pcDNA3.4-His-MBP-monkey TfR gene expression vector were constructed.

### (2) Preparation of TfR Proteins

A His-MBP-human TfR protein and a His-MBP-monkey TfR protein were produced and purified with the following procedure using an Expi293 expression system (Thermo). Each expression vector was transfected into Expi293F cells, and after four days, the culture medium was collected, and clarified by removing the cells. The purification from the clarified supernatant was performed using an amylose resin (New England Biolabs), and the purified product was buffer-exchanged with PBS by dialysis or desalting column.

(3) Acquisition of scFv

By screening using the human TfR protein and a fully synthetic human antibody phage library, a human antibody fragment (scFv) that specifically binds to human TfR was obtained according to the following procedure.

The His-MBP-human TfR protein was immobilized on Dynabeads M-280 Tosylactivated magnetic beads (Thermo), holo human transferrin (FUJIFILM Wako Pure) was added, and the mixture was stirred for one hour to form a complex. After washing with PBS, the complex was incubated with a fully synthetic human antibody phage library for one hour, and unbound phage was removed by a series of washing cycles using 0.1% Tween 20 (v/v)/PBS. To enrich clones exhibiting pH-dependent binding, phage particles remaining on the magnetic beads were eluted with 0.1 M citrate buffer (pH 6.0) and then used to infect E. coli TG1 host cells to amplify the phage. This panning procedure was further performed once more using the amplified phage.

After two rounds of panning, single colonies were picked from E. coli infected with phage and inoculated into medium in a 96-well plate. After culturing the E. coli to the logarithmic growth phase, IPTG was added, followed by shaking culture at 30° C. to induce production of an scFv fused with a FLAG tag at the C-terminus thereof (scFv-FLAG). On the next day, the E. coli culture was centrifuged, and the culture supernatant containing scFv-FLAG was used to evaluate the binding properties to the human TfR/human transferrin complex by ELISA as described below.

Anti-FLAG M2 antibodies (Sigma-Aldrich) were added to each well of a 96-well plate and incubated overnight at 4° C. On the next day, the His-MBP-human TfR protein and holo human transferrin were mixed and incubated at room temperature for one hour to form a human TfR/human transferrin complex. After washing the plate three times with 0.02% Tween 20/PBS, the human TfR/human transferrin complex and the scFv-FLAG-containing E. coli culture supernatant were mixed, added to the wells, and incubated for two hours at room temperature. After washing three times with 0.02% Tween 20/PBS, either 0.1 M citrate buffer (pH 6.0) or PBS (pH 7.4) was added and incubated for 30 minutes at room temperature. The remaining amount of the human TfR/human transferrin complex bound to scFv-FLAG was detected using a horseradish peroxidase (HRP)-labeled anti-His antibody (Medical & Biological Laboratories Co., Ltd.). By comparing the absorbance of wells washed at pH 6.0 with that of wells washed at pH 7.4, clones exhibiting pH-dependent binding were selected as scFv-FLAG clones susceptible to dissociation under the pH 6.0 washing condition. Sequence analysis of the clones exhibiting pH-dependent binding revealed the DNA sequence and amino acid sequence of aT04-scFv (DNA sequence: SEQ ID NO: 18; amino acid sequence: SEQ ID NO: 19). The CDRs of aT04-scFv were determined according to the Kabat definition. The amino acid sequences of the CDRs of the heavy and light chains, as well as the amino acid sequences of the heavy chain variable region (VH) and light chain variable region (VL), are shown in Tables 3 and 4, respectively.

TABLE 3
Amino acid sequences of CDRs of aT04

| Name | Sequence | SEQ ID NO |
|---|---|---|
| HCDR1 | SYAMS | 1 |
| HCDR2 | AISGSGGSTYYADSVKG | 7 |
| HCDR3 | MKSTRHWIDD | 3 |
| LCDR1 | QGDSLRSYYAS | 4 |
| LCDR2 | GKNNRPS | 5 |
| LCDR3 | QSYDSSSYHYV | 6 |

TABLE 4
Amino acid sequences of heavy and light chain
variable regions of aT04

| | SEQ ID | |
|---|---|---|
| Name | NO | Sequence |
| aT04-VH | 64 | EVQLLESGGGLVQPGGSLRLSCAASGFTFSSYA |
| | | MSWVRQAPGKGLEWVSAISGSGGSTYYADSVK |
| | | GRFTISRDNSKNTLYLQMNSLRAEDTAVYYCAK |
| | | MKSTRHWIDDWGQGTLVTVSS |
| aT04-VL | 65 | SSELTQDPAVSVALGQTVRITCQGDSLRSYYAS |
| | | WYQQKPGQAPVLVIYGKNNRPSGIPDRFSGSSS |
| | | GNTASLTITGAQAEDEADYYCQSYDSSSYHYVF |
| | | GGGTKLTVL |

### (4) Construction of Bispecific Anti-Human TfR/EphA4 Antibody Gene Expression Vector

The DNA sequences that encode the first heavy chain, the second heavy chain, and the light chain of the anti-human EphA4 agonist antibody #12 were cloned into a pcDNA3.4 vector. In this case, as a knob-into-hole heterodimerization technique, a knob substitution in the CH3 domain of the first heavy chain (threonine at residue 366 according to EU numbering is substituted with tryptophan (T366W)) and a corresponding hole substitution in the CH3 domain of the second heavy chain (threonine at residue 366 according to EU numbering is substituted with serine (T366S), leucine at residue 368 is substituted with alanine (L368A), and tyrosine at residue 407 is substituted with valine (Y407V)), as well as two additional cysteine residues are introduced (S354C in the CH3 domain of the first heavy chain (serine at residue 354 according to EU numbering is substituted with cysteine) and Y349C in the CH3 domain of the second heavy chain (tyrosine at residue 349 according to EU numbering is substituted with cysteine)) were used. Further, to the DNA sequence that encodes the second heavy chain, a DNA sequence that encodes a G4S linker (GGGGSAAA (SEQ ID NO: 79)) and a restriction enzyme site for introducing scFv were introduced at the C-terminus. Next, a DNA sequence encoding aT04-scFv was inserted into the vector expressing the second heavy chain gene. Accordingly, vectors each expressing a first heavy chain gene (DNA sequence: SEQ ID NO: 20, amino acid sequence: SEQ ID NO: 21), a second heavy chain and scFv gene (DNA sequence: SEQ ID NO: 22, amino acid sequence: SEQ ID NO: 23), and a light chain gene (DNA sequence: SEQ ID NO: 24, amino acid sequence: SEQ ID NO: 25) of the bispecific anti-human TfR/EphA4 antibody (#12-aT04mv) were constructed. Note that the light chain of the anti-human EphA4 agonist antibody #12 is the same as the light chain of #12-aT04mv, and the heavy chain consists of the amino acid sequence of SEQ ID NO: 27, and the DNA encoding this consists of the DNA sequence of SEQ ID NO: 26.

### (5) Production and Purification of Bispecific Anti-Human TfR/EphA4 Antibody

Using an ExpiCHO expression system (Thermo), the vector expressing the second heavy chain and scFv gene (SEQ ID NO: 23) of #12-aT04mv, the vector expressing the first heavy chain gene of SEQ ID NO: 21, and the vector expressing the light chain gene of SEQ ID NO: 25 were transfected into ExpiCHO-S cells (Thermo). After 11 days, the culture medium was collected and clarified by removing the cells. The clarified supernatant was purified using a HiTrap MabSelect SuRe column (Cytiva, Inc.), and from its eluate, the bispecific anti-human TfR/EphA4 antibody #12-aT04mv was fractionated and collected by size-exclusion chromatography (HiLoad 16/600 Superdex 200 μg, Cytiva, Inc.) using PBS as the mobile phase.

### Experimental Example 2: pH-Dependent Binding of Bispecific Anti-Human TfR/EphA4 Antibody to Human TfR

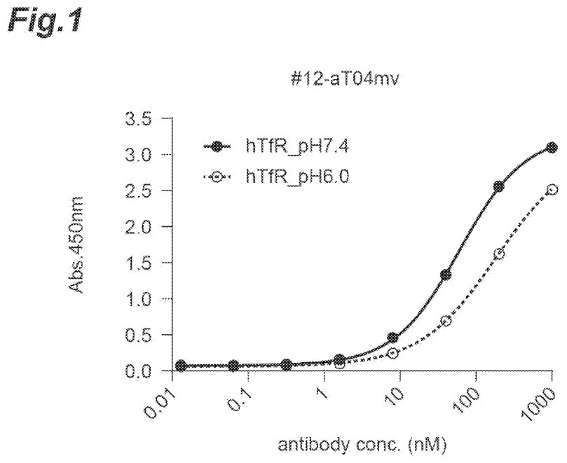

The pH-dependent binding of #12-aT04mv to human TfR was evaluated by ELISA as described below. His-MBP-human TfR protein was added to a 96-well plate on which an anti-His antibody (R&D Systems, Inc.) was immobilized, and incubated at room temperature for one hour to immobilize the His-MBP-human TfR protein on the plate. After washing the plate three times with 0.02% Tween 20/PBS, #12-aT04mv was added to each well and incubated at room temperature for one hour. After washing three times with 0.02% Tween 20/PBS, 0.1 M citrate buffer (pH 6.0) or PBS (pH 7.4) was added and it was incubated at room temperature for 30 minutes. The remaining amount of #12-aT04mv bound to human TfR was detected using an HRP-labeled anti-human IgG antibody (Jackson ImmunoResearch Inc.). The absorbance at 450 nm for each antibody concentration is shown in FIG. 1. From FIG. 1, it was found that #12-aT04mv exhibits pH-dependent binding properties.

In addition, the EC50 value of the group incubated at pH 6.0 was 184.2 nM, and the EC50 value of the group incubated at pH 7.4 was 60.5 nM. From these ratios, it was suggested that #12-aT04mv is 3.0-fold more susceptible to dissociation from human TfR under pH 6.0 conditions than under pH 7.4 conditions.

### Experimental Example 3: Binding Affinity of Bispecific Anti-Human TfR/EphA4 Antibody to Human and Monkey TfR

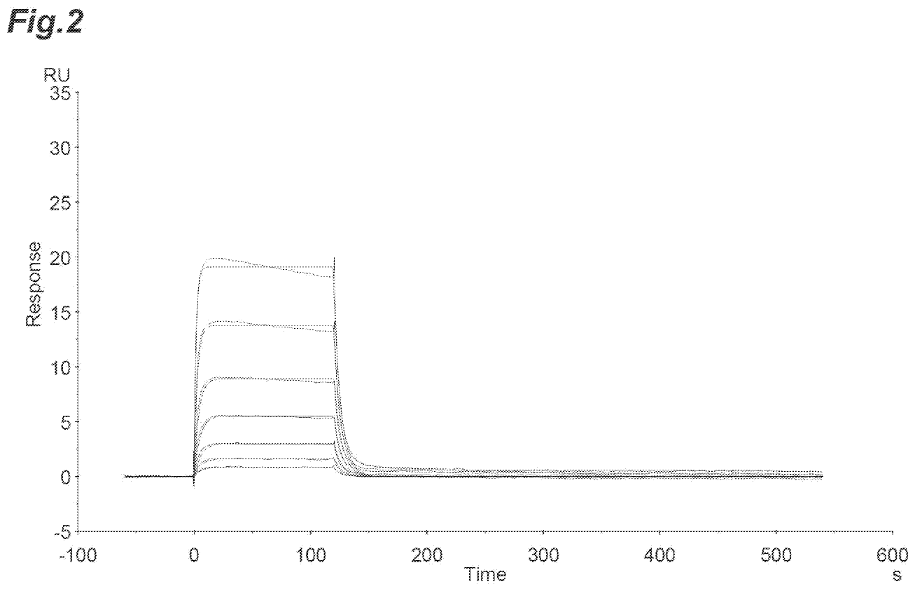

The measurement of binding affinity of #12-aT04mv to human and monkey TfR was performed by the surface plasmon resonance (SPR) method (Biacore T200, Cytiva, Inc.) as described below. His-MBP-hTfR protein and His-MBP-monkey TfR protein diluted with HBS-EP+ buffer were injected and captured on an anti-His antibody immobilized on a Series S CM5 sensor chip. Immobilization of the anti-His antibody was performed by a standard amine-coupling method according to the vendor's manual. Next, #12-aT04mv prepared in various dilution series with HBS-EP+ buffer was sequentially injected, and binding reactions were observed for 120 seconds (association phase) and 420 seconds (dissociation phase). Analysis of sensorgram using BIAevaluation Software ver. 2.0 (Cytiva, Inc.) was performed, and the binding affinity (KD value) of #12-aT04mv was calculated using a 1:1 binding model. The results are shown in Table 5 below. The binding KD value of #12-aT04mv to human TfR was 1.90×10⁻⁷ M, and binding to monkey TfR could not be confirmed. FIG. 2 shows the binding response curves of #12-aT04mv at various dilution series to human TfR.

TABLE 5

| | ka | kd | KD |
|---|---|---|---|
| Sample | (1/Ms) | (1/s) | (M) |
| #12-aT04mv | 8.23 × 10⁵ | 1.57 × 10⁻¹ | 1.90 × 10⁻⁷ |

### Experimental Example 4: Identification of Binding Domain of Bispecific Anti-Human TfR/EphA4 Antibody

### (1) TfR Chimeric Gene Synthesis and Cloning

A DNA sequence that encodes a human-mouse TfR chimera (h/m TfR 184-385, SEQ ID NO: 253), in which the amino acid sequence at positions 186 to 387 of mouse TfR (GenBank Accession No. NP_035768.1) was replaced with the corresponding amino acid sequence at positions 184 to 385 of human TfR, was synthesized in its entirety by GenScript. The DNA sequence encoding the TfR amino acid sequences was cloned into a pCSX4 vector in which a DNA sequence encoding a puromycin resistance gene had been previously incorporated, thereby constructing a vector for establishing pCSX4-human-mouse TfR chimera-producing cells.

(2) IgG Conversion of aT04, and Production and Purification

By subcloning the DNA sequences encoding the variable regions of aT04-scFv into vectors expressing human antibody heavy chain and light chain constant regions, the clones were converted from an scFv format to an IgG format (aT04-IgG; heavy chain DNA: SEQ ID NO: 28, heavy chain amino acid sequence: SEQ ID NO: 29; light chain DNA: SEQ ID NO: 30, light chain amino acid sequence: SEQ ID NO: 31). The heavy chain of this IgG is a chimeric IgG heavy chain composed of a human IgG1-derived CH1 and hinge region, and human IgG2-derived CH2 and CH3 regions containing V234A and G237A mutations. Using the ExpiCHO expression system (Thermo), two pcDNA3.4 vectors containing the DNA sequences encoding the heavy and light chains of aT04-IgG were transfected into ExpiCHO-S cells (Thermo). The culture supernatant was collected, and aT04-IgG was obtained using MabSelect SuRe (Cytiva, Inc.).

### (3) Selection of TfR Chimeric Gene-Expressing Cells

Lenti-X293T cells (Clontech Laboratories, Inc.) were seeded into a 6-well plate and cultured at 37° C. for 7 hours. The above human-mouse TfR chimeric gene expression vector was transfected into Lenti-X293T cells together with a pGP vector (Takara Bio Inc.) and a pE-eco vector (Takara Bio Inc.) using the Lipofectamine 3000 Transfection Kit (Thermo). After culturing for three days, the culture supernatant containing the produced retrovirus particles was collected and clarified by removing the cells. The clarified retrovirus-containing supernatant, together with polybrene and 2-mercaptoethanol, was used to transduce B300.19 cells. On the next day, puromycin was added, and the cells were further cultured for six days to remove non-transduced cells.

### (4) Flow Cytometry Analysis

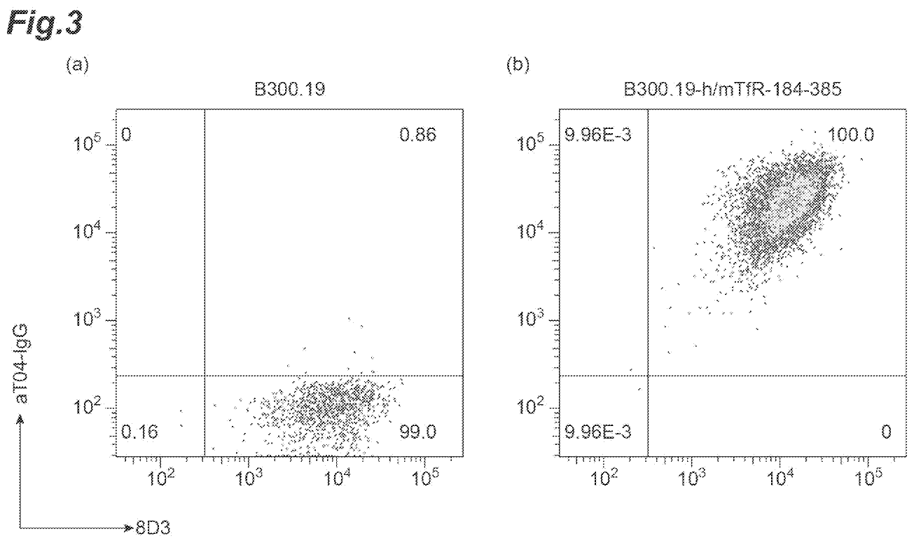

Flow cytometry analysis using B300.19 cells producing the human-mouse TfR chimera (h/m TfR 184-385) was performed according to the following procedure. Human-mouse TfR chimera-producing B300.19 cells were suspended in 10% mouse serum/1% FBS (NICHIREI CORPORATION)/1 mM EDTA/PBS (containing 0.02% sodium azide (FUJIFILM Wako Pure)), blocked at 4° C. for 20 minutes, and then incubated at 4° C. for 20 minutes with rat anti-mouse TfR antibody clone 8D3 (Novus Biologicals, LLC) and aT04-IgG prepared in the same buffer composition. Cells were washed three times, incubated with a fluorescent-labeled secondary antibody (Jackson ImmunoResearch Inc.) at 4° C. for 20 minutes and then washed again three times, and fluorescence signals were detected using an LSRFortessa X-20 (BD). The results of the flow cytometry analysis are shown in FIG. 3.

aT04-IgG did not exhibit reactivity toward B300.19 cells that did not express the human-mouse TfR chimera. In contrast, aT04-IgG exhibited reactivity toward B300.19-h/m TfR-184-385 cells expressing the human-mouse TfR chimera of SEQ ID NO: 253. Therefore, it was found that aT04-IgG recognizes the amino acid sequence region at positions 184 to 385 of human TfR.

### Experimental Example 5: Evaluation of Brain-Penetrating Properties of Bispecific Anti-Human TfR/EphA4 Antibody

### (1) Establishment of Human TfR Knock-In Mouse

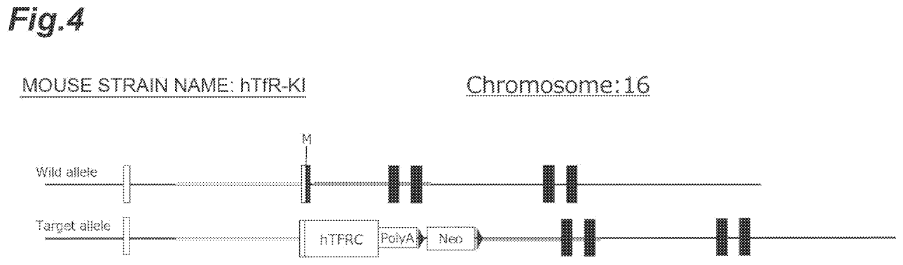

To generate knock-in mice, a method for producing aggregated embryos using mouse 8-cell stage embryos and genetically engineered ES cells, was used. Aggregated embryos of MCH (ICR) mouse-derived 8-cell stage embryos and ES cells subjected to homologous recombination of human TFRC genes into mouse Tfrc genes were produced, and embryo transfer was performed into pseudopregnant female mice, and thereby a human TfR knock-in mouse (hTfR-KI mouse) strain was established. Specifically, a targeting vector was produced, and it was introduced into ES cells using an electroporation method. As a result, the homologous recombination occurred between mouse Tfrc genes and the targeting vector, and recombinant ES cells were produced (FIG. 4). Subsequently, by the method for producing aggregated embryos with MCH (ICR) mouse-derived 8-cell stage embryos using recombinant ES cells, chimeric embryos into which ES cells were introduced were generated, which were embryo transferred into pseudopregnant female mice. Male chimeric mice obtained by embryo transfer were crossed with wild-type females (C57BL/6J) to generate F1 generation heterozygous mice. F1 heterozygous male mice were back-crossed with wild-type female again to generate N2 generation heterozygous mice, and the strain was established. By sibling crossing of established heterozygous mice, homozygous mouse hTfR-KI mice were generated.

(2) Sample Preparation from Bispecific Anti-human TfR/EphA4 Antibody treated Animals

Sample preparation from #12-aT04mv treated animals was performed according to the following steps. To 8-week-old male homozygous hTfR-KI mice, #12 or #12-aT04mv was administered via the tail vein at a dose of 133 nmol/kg (10 mL/kg). After 24 hours, blood was collected under anesthesia, followed by exsanguination via incision of the right atrial appendage and transcardial perfusion with heparin-containing PBS. A hippocampus on one side and a cerebral hemisphere from one side were collected. The blood was, after collection, centrifuged at 3,000 rpm for 15 minutes at 4° C., and the supernatant was collected as plasma. For the hippocampus, the weight of the wet tissues was measured and homogenized with a BioMasher (Nippi Inc.), and then tissue homogenate was prepared. To a portion of the prepared hippocampal tissue homogenate, NP-40 at the final concentration of 1% was added and incubated for 60 minutes at 4° C. and then centrifuged at 15,000 rpm for 10 minutes at 4° C. to collect the supernatant. The cerebral hemisphere was dipped into 2% paraformaldehyde (TAAB Laboratories Equipment Ltd.)/0.1 M PB and agitated overnight at 4° C. The cerebral hemisphere in which immersion fixation had been completed was substituted with 10% sucrose/0.1 M PB and 20% sucrose/0.1 M PB, and then embedded in Tissue-Tek O. C. T. Compound (Sakura Finetek Japan Co., Ltd.)/20% sucrose and frozen in liquid nitrogen.

### (3) Measurement of Bispecific Anti-Human TfR/EphA4 Antibody Concentration in the Tissues

Measurement of concentrations of #12 and #12-aT04mv in plasma and hippocampal tissue lysate collected according to the above-described method was performed according to the following steps. A 96-well plate was coated with a donkey anti-human IgG-Fcγ chain-specific F(ab′)₂ antibody (Jackson ImmunoResearch Inc.). After incubating for 1 hour at room temperature or overnight at 4° C., washing was performed with 0.02% Tween20/PBS three times. After blocking the wells at room temperature for one hour with 1% Block Ace (DS Pharma Biomedical Co., Ltd.), plasma or hippocampal tissue lysate diluted with 0.4% Block Ace was added and incubated at room temperature for 2 hours. After washing three times, a horseradish peroxidase-labeled goat anti-human IgG-Fcγ chain-specific F(ab′)2 antibody (Jackson ImmunoResearch Inc.) was added and incubated at room temperature for one hour. After washing three times, TMBZ (3,3′,5,5′-tetramethyl benzidine, SeraCare) solution was added to the well and incubated at room temperature for 15 minutes. An equivalent amount of quenching solution (2N sulfuric acid) was added to the well, and the absorbance at 450 nm and 650 nm was measured by a microplate reader (Thermo).

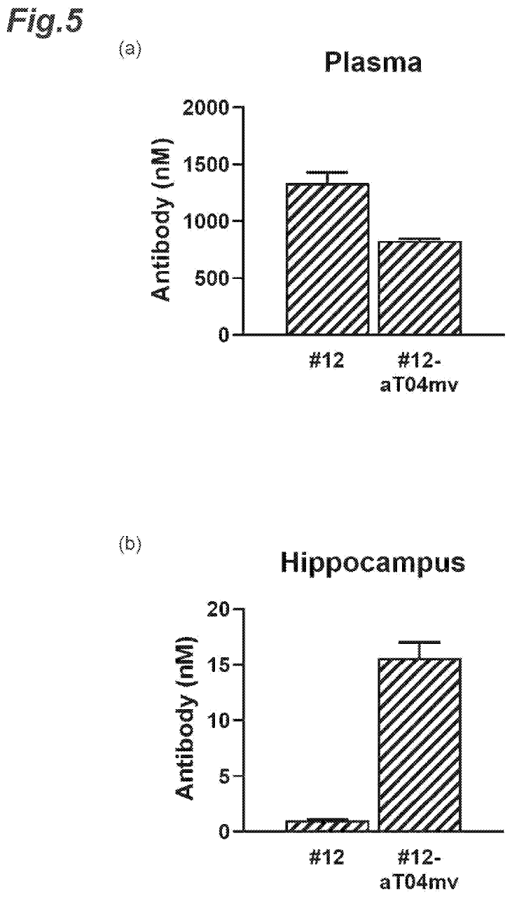

The results of the concentration calculations based on the measured absorbance are shown in FIG. 5. It was found from FIG. 5 that the concentration in the hippocampus of individuals treated with #12-aT04mv was markedly higher than that of individuals treated #12. Accordingly, it was found that #12-aT04mv has enhanced brain penetration capability.

### (4) Intracerebral Distribution of Bispecific Anti-Human TfR/EphA4 Antibody

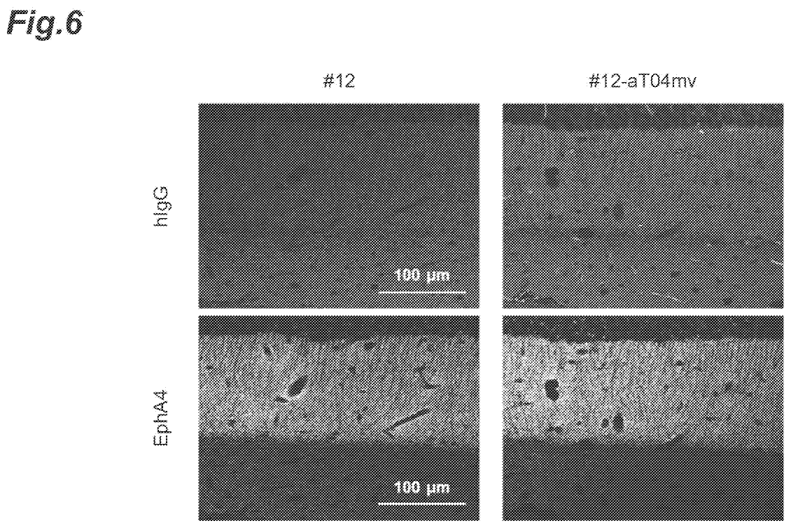

Confirmation of the intracerebral distribution of #12-aT04mv was performed according to the following steps. A section was prepared from the O. C. T. Compound-embedded frozen tissue prepared in (2), in a thickness of 8 μM using Cryostat CM1860 (Leica), mounted onto a glass slide, dried by a cold air stream, and then enclosed in a sealed bag and stored at −80° C. The glass slide for use in immunostaining was taken out from −80° C. and air dried by a cold air stream, and then washed with PBS, immersed in 1% BSA/10% normal donkey serum (Jackson ImmunoResearch Inc.)/0.5% Triton (trademark) X-100/PBS solution, and subjected to blocking procedure for one hour. A goat anti-mouse EphA4 antibody (R&D) and fluorescent-labeled donkey anti-human IgG antibody (Jackson ImmunoResearch Inc.), which were diluted in 1% BSA/3% normal donkey serum/0.5% Triton (trademark) X-100/PBS solution, were left to react overnight at 4° C. After washing with PBS three times and reacting with a fluorescent-labeled secondary antibody (Millipore) for one hour, the reacted tissue sections were washed again with PBS three times, Prolong Gold antifade reagent (Molecular probes) was placed on the section and sealed, and observed by LSM800 (Zeiss) to obtain images (FIG. 6).

The intracerebral distributions of the antibodies were evaluated in the SR layer of the hippocampus CA1 region where EphA4, the target molecule of #12, is highly produced. As a result, higher signals of human IgG were obtained in #12-aT04mv-treated animals compared to #12-treated animals. Accordingly, it was found that #12-aT04mv is present in a greater amount in the brain parenchyma than #12.

### Experimental Example 6: Production of Human TfR Antibody Mutants

(1) Obtaining of scFv

In order to impart cross-reactivity with monkey TfR to aT04-scFv, aT04-scFv mutants were generated from the mutagenesis phage libraries described below. Random mutations were introduced into the sites corresponding to heavy chain CDR1 and heavy chain CDR2 within the DNA sequence encoding aT04-scFv, and used to construct an scFv gene library. This library was cloned into a pUC119 vector (Takara Bio Inc.) in which a DNA sequence encoding the Fd phage pIII protein had been previously incorporated, and transformed into E. coli TG1 host cells. The transformed E. coli was amplified and infected with M13KO7 helper phage (Agilent Technologies, Inc.), thereby producing a mutant scFv-displaying phage library. Using this phage library and monkey and human TfR proteins, the panning and screening described in Experimental Example 1 were performed, and the DNA sequence and amino acid sequence of aT04m02-scFv were identified as a mutant exhibiting cross-reactivity to both human TfR and monkey TfR (DNA sequence: SEQ ID NO: 32; amino acid sequence: SEQ ID NO: 33). Further, in order to identify residues contributing to cross-reactivity with monkey TfR, aT04m0201 through aT04m0207-scFvs were designed, each having one of the seven mutations introduced in aT04m02 individually reverted to the amino acid used in the template aT04 (DNA sequences: SEQ ID NOs: 34, 36, 38, 40, 42, 44, and 46; amino acid sequences: SEQ ID NOs: 35, 37, 39, 41, 43, 45, and 47). The amino acid sequences of the CDRs of the aT04-mutant scFvs are shown in Table 6 below.

TABLE 6

| | | |
|---|---|---|
| Name | Sequence | SEQ ID NO |

Amino acid sequences of CDRs of aT04m02-scFv (SEQ ID NO: 33)

| | | |
|---|---|---|
| HCDR1 | DYAMS | 2 |
| HCDR2 | AIRWHSSWTYYADSVKG | 8 |
| HCDR3 | MKSTRHWIDD | 3 |
| LCDR1 | QGDSLRSYYAS | 4 |
| LCDR2 | GKNNRPS | 5 |
| LCDR3 | QSYDSSSYHYV | 6 |

Amino acid sequences of CDRs of aT04m0201-scFv (SEQ ID NO: 35)

| | | |
|---|---|---|
| HCDR1 | SYAMS | 1 |
| HCDR2 | AIRWHSSWTYYADSVKG | 8 |
| HCDR3 | MKSTRHWIDD | 3 |
| LCDR1 | QGDSLRSYYAS | 4 |
| LCDR2 | GKNNRPS | 5 |
| LCDR3 | QSYDSSSYHYV | 6 |

Amino acid sequences of CDRs of aT04m0202-scFv (SEQ ID NO: 37)

| | | |
|---|---|---|
| HCDR1 | DYAMS | 2 |
| HCDR2 | AISWHSSWTYYADSVKG | 9 |
| HCDR3 | MKSTRHWIDD | 3 |
| LCDR1 | QGDSLRSYYAS | 4 |
| LCDR2 | GKNNRPS | 5 |
| LCDR3 | QSYDSSSYHYV | 6 |

Amino acid sequences of CDRs of aT04m0203-scFv (SEQ ID NO: 39)

| | | |
|---|---|---|
| HCDR1 | DYAMS | 2 |
| HCDR2 | AIRGHSSWTYYADSVKG | 10 |
| HCDR3 | MKSTRHWIDD | 3 |
| LCDR1 | QGDSLRSYYAS | 4 |
| LCDR2 | GKNNRPS | 5 |
| LCDR3 | QSYDSSSYHYV | 6 |

Amino acid sequences of CDRs of aT04m0204-scFv (SEQ ID NO: 41)

| | | |
|---|---|---|
| HCDR1 | DYAMS | 2 |
| HCDR2 | AIRWSSSWTYYADSVKG | 11 |
| HCDR3 | MKSTRHWIDD | 3 |
| LCDR1 | QGDSLRSYYAS | 4 |
| LCDR2 | GKNNRPS | 5 |
| LCDR3 | QSYDSSSYHYV | 6 |

Amino acid sequences of CDRs of aT04m0205-scFv (SEQ ID NO: 43)

| | | |
|---|---|---|
| HCDR1 | DYAMS | 2 |
| HCDR2 | AIRWHGSWTYYADSVKG | 12 |
| HCDR3 | MKSTRHWIDD | 3 |
| LCDR1 | QGDSLRSYYAS | 4 |
| LCDR2 | GKNNRPS | 5 |
| LCDR3 | QSYDSSSYHYV | 6 |

Amino acid sequences of CDRs of aT04m0206-scFv (SEQ ID NO: 45)

| | | |
|---|---|---|
| HCDR1 | DYAMS | 2 |
| HCDR2 | AIRWHSGWTYYADSVKG | 13 |
| HCDR3 | MKSTRHWIDD | 3 |
| LCDR1 | QGDSLRSYYAS | 4 |
| LCDR2 | GKNNRPS | 5 |
| LCDR3 | QSYDSSSYHYV | 6 |

Amino acid sequences of CDRs of aT04m0207-scFv (SEQ ID NO: 47)

| | | |
|---|---|---|
| HCDR1 | DYAMS | 2 |
| HCDR2 | AIRWHSSSTYYADSVKG | 14 |
| HCDR3 | MKSTRHWIDD | 3 |
| LCDR1 | QGDSLRSYYAS | 4 |
| LCDR2 | GKNNRPS | 5 |
| LCDR3 | QSYDSSSYHYV | 6 |

The amino acid sequences of the heavy chain variable regions (VH) of aT04-mutant scFvs are shown in Table 7 below. Note that the sequences of the light chain variable regions (VL) are identical to the sequence of aT04-VL (SEQ ID NO: 65).

TABLE 7

| | SEQ ID | |
|---|---|---|
| Name | NO | Sequence |
| aT04m02-VH | 66 | EVQLLESGGGLVQPGGSLRLSCAASGFTFSDY |
| | | AMSWVRQAPGKGLEWVSAIRWHSSWTYYAD |
| | | SVKGRFTISRDNSKNTLYLQMNSLRAEDTAVY |
| | | YCAKMKSTRHWIDDWGQGTLVTVSS |
| aT04m0201- | 67 | EVQLLESGGGLVQPGGSLRLSCAASGFTFSSY |
| VH | | AMSWVRQAPGKGLEWVSAIRWHSSWTYYAD |
| | | SVKGRFTISRDNSKNTLYLQMNSLRAEDTAVY |
| | | YCAKMKSTRHWIDDWGQGTLVTVSS |
| aT04m0202- | 68 | EVQLLESGGGLVQPGGSLRLSCAASGFTFSDY |
| VH | | AMSWVRQAPGKGLEWVSAISWHSSWTYYAD |
| | | SVKGRFTISRDNSKNTLYLQMNSLRAEDTAVY |
| | | YCAKMKSTRHWIDDWGQGTLVTVSS |
| aT04m0203- | 69 | EVQLLESGGGLVQPGGSLRLSCAASGFTFSDY |
| VH | | AMSWVRQAPGKGLEWVSAIRGHSSWTYYAD |
| | | SVKGRFTISRDNSKNTLYLQMNSLRAEDTAVY |
| | | YCAKMKSTRHWIDDWGQGTLVTVSS |
| aT04m0204- | 70 | EVQLLESGGGLVQPGGSLRLSCAASGFTFSDY |
| VH | | AMSWVRQAPGKGLEWVSAIRWSSSWTYYAD |
| | | SVKGRFTISRDNSKNTLYLQMNSLRAEDTAVY |
| | | YCAKMKSTRHWIDDWGQGTLVTVSS |
| aT04m0205- | 71 | EVQLLESGGGLVQPGGSLRLSCAASGFTFSDY |
| VH | | AMSWVRQAPGKGLEWVSAIRWHGSWTYYA |
| | | DSVKGRFTISRDNSKNTLYLQMNSLRAEDTAV |
| | | YYCAKMKSTRHWIDDWGQGTLVTVSS |
| aT04m0206- | 72 | EVQLLESGGGLVQPGGSLRLSCAASGFTFSDY |
| VH | | AMSWVRQAPGKGLEWVSAIRWHSGWTYYA |
| | | DSVKGRFTISRDNSKNTLYLQMNSLRAEDTAV |
| | | YYCAKMKSTRHWIDDWGQGTLVTVSS |
| aT04m0207- | 73 | EVQLLESGGGLVQPGGSLRLSCAASGFTFSDY |
| VH | | AMSWVRQAPGKGLEWVSAIRWHSSSTYYAD |
| | | SVKGRFTISRDNSKNTLYLQMNSLRAEDTAVY |
| | | YCAKMKSTRHWIDDWGQGTLVTVSS |

(2) Cloning of scFv Gene

Similarly to Experimental Example 1, DNA sequences encoding aT04m02-scFv and aT04m0201-scFv through aT04m0207-scFv were 5 inserted into a vector expressing the second heavy chain of the anti-EphA4 agonist antibody #12. Accordingly, vectors expressing the second heavy chain and scFv genes (DNA sequences: SEQ ID NOs: 48, 50, 52, 54, 56, 58, 60, and 62; amino acid sequences: SEQ ID NOs: 49, 51, 53, 55, 57, 61, and 63) of the bispecific anti-human TfR/EphA4 antibodies (#12-aT04m02mv and #12-aT04m0201mv through #12-aT04m0207mv) were constructed.

### (3) Production and Purification of Bispecific Anti-Human TfR/EphA4 Mutant Antibody

Using the ExpiCHO expression system (Thermo), the vector expressing genes encoding the second heavy chain and scFv containing the respective amino acid sequences of #12-aT04m02mv and #12-aT04m0201mv through #12-aT04m0207mv, the vector expressing the first heavy chain gene consisting of the amino acid sequence of SEQ ID NO: 21, and the vector expressing the light chain gene of SEQ ID NO: 25 were transfected into ExpiCHO-S cells (Thermo). After 9 or 11 days, the culture media were collected and clarified, and #12-aT04m02mv and #12-aT04m0201mv through #12-aT04m0207mv were purified using MabSelect SuRe pcc (Cytiva, Inc.).

### Experimental Example 7: pH-Dependent Binding of Bispecific Anti-Human TfR/EphA4 Mutant Antibody to Human TfR

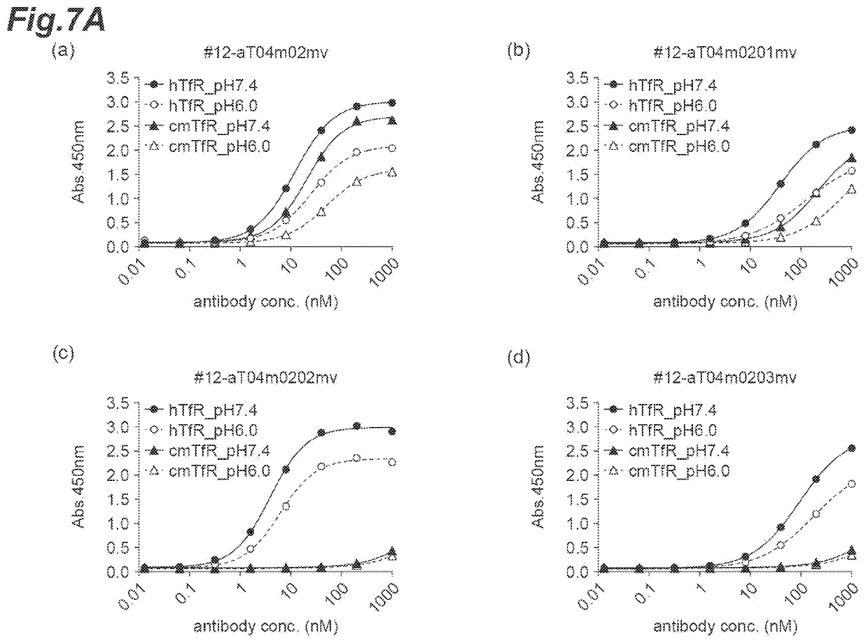

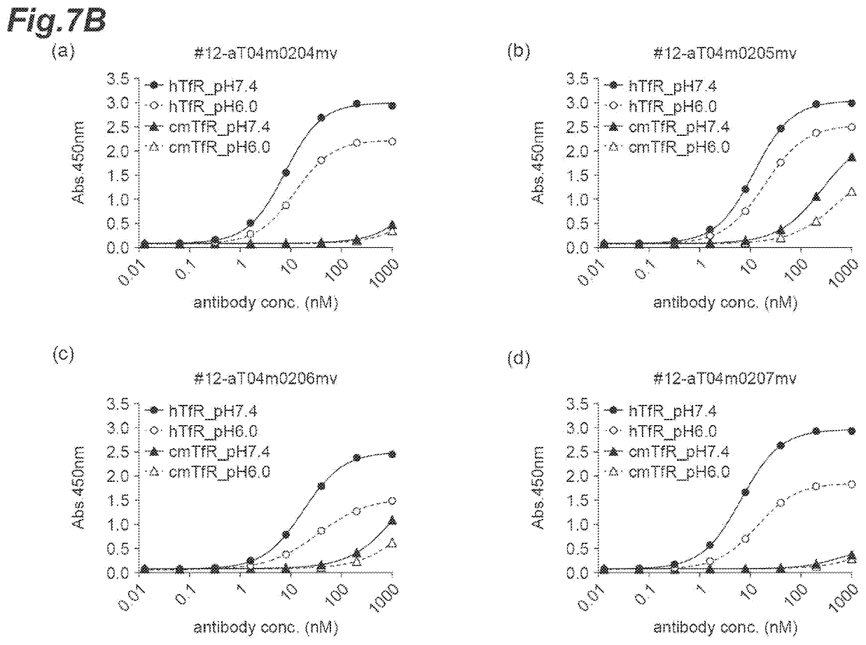

The pH-dependent binding of #12-aT04m02mv and #12-aT04m0201mv through #12-aT04m0207mv to human and monkey TfR was evaluated according to the ELISA procedure described in Experimental Example 2. The results are shown in FIGS. 7A and 7B.

From FIGS. 7A and 7B, binding to monkey TfR was confirmed for #12-aT04m02mv, #12-aT04m0201mv, #12-aT04m0205mv, and #12-aT04m0206mv. For these four antibodies, it was suggested that they are 2.1-fold, 3.2-fold, 1.6-fold, and 2.0-fold more susceptible to dissociation from human TfR, and 2.4-fold, 3.1-fold, 2.0-fold, and 1.9-fold more susceptible to dissociation from monkey TfR under pH 6.0 conditions than under pH 7.4 conditions. In addition, even for #12-aT04m0202mv, #12-aT04m0203mv, #12-aT04m0204mv, and #12-aT04m0207mv, which did not bind to monkey TfR, it was suggested that they bind to human TfR and are 1.6-fold, 1.8-fold, 1.6-fold, and 2.0-fold more susceptible to dissociation from human TfR under pH 6.0 conditions than under pH 7.4 conditions.

### Experimental Example 8: Binding Affinity of Bispecific Anti-Human TfR/EphA4 Mutant Antibody to Human TfR

The measurement of binding affinity of #12-aT04m02mv, #12-aT04m0201mv, #12-aT04m0205mv, and #12-aT04m0206mv, whose binding to monkey TfR was observed, to human and monkey TfR was performed according to the steps of the surface plasmon resonance described in Experimental Example 3. The results are shown in Table 8.

The binding KD values of #12-aT04m02mv, #12-aT04m0201mv, and #12-aT04m0205mv to human TfR were comparable to the binding KD value of #12-aT04mv described in FIG. 2 and Table 5 to human TfR (Table 8). Also, it was suggested that, from the binding KD values of #12-aT04m02mv and #12-aT04m0201mv to monkey TfR, these two antibodies have the similar binding affinity to human and monkey TfR.

TABLE 8

| | Human TfR (hTfR) | | | Monkey TfR (cmTfR) | | |
|---|---|---|---|---|---|---|
| Sample | ka (1/Ms) | kd (1/s) | KD (M) | ka (1/Ms) | kd (1/s) | KD (M) |
| #12- | 7.83E+04 | 7.35E−03 | 9.39E−08 | 2.02E+05 | 5.23E−02 | 2.59E−07 |
| aT04m02mv | | | | | | |
| #12- | 1.03E+05 | 1.53E−02 | 1.49E−07 | 3.89E+05 | 2.67E−01 | 6.87E−07 |
| aT04m0201mv | | | | | | |
| #12- | 3.53E+05 | 3.13E−02 | 8.88E−08 | 1.52E+05 | 5.79E−01 | 3.82E−06 |
| aT04m0205mv | | | | | | |
| #12- | 2.36E+05 | 1.05E−02 | 4.44E−08 | 1.47E+05 | 3.48E−01 | 2.36E−06 |
| aT04m0206mv | | | | | | |

### Experimental Example 9: Evaluation of Brain-Penetrating Properties of Bispecific Anti-Human TfR (Cross-Reactivity with Monkey)/EphA4 Mutant Antibody

(1) Sample Preparation from Bispecific Anti-Human TfR (Cross-Reactivity with Monkey)/EphA4 Antibody Treated Animals

Sample preparation from animals treated with #12-aT04m02mv, #12-aT04m0201mv, #12-aT04m0205mv, and #12-aT04m0206mv, whose binding to monkey TfR was observed above, was performed according to the following steps. To 8-week-old male homozygous hTfR-KI mice, #12-aT04m02mv, #12-aT04m0201mv, #12-aT04m0205mv, or #12-aT04m0206mv was administered via the tail vein at a dose of 133 nmol/kg (10 mL/kg). After 24 hours and 168 hours, blood was collected under anesthesia with 2.5% isoflurane inhalational anesthesia and a triple mixed anesthesia for mouse (by subcutaneous administration), and then exsanguination via incision of the right atrial appendage and transcardial perfusion with heparin-containing PBS were performed to collect the hippocampus on one side and the cerebral hemisphere on one side. The blood was, after collection, centrifuged at 3,000 rpm for 15 minutes at 4° C., and the supernatant was collected as plasma. For the hippocampus, the weight of the wet tissues was measured and homogenized with a BioMasher (Nippi Inc.), and then tissue homogenate was prepared. To a portion of the prepared hippocampal tissue homogenate, NP-40 at the final concentration of 1% was added and incubated at 4° C. for 60 minutes, and then centrifuged at 15,000 rpm for 10 minutes at 4° C. to collect the supernatant. The cerebral hemisphere was dipped into 2% paraformaldehyde (TAAB Laboratories Equipment Ltd.)/0.1 M PB and agitated overnight at 4° C. The cerebral hemisphere in which immersion fixation had been completed was substituted with 10% sucrose/0.1 M PB and 20% sucrose/0.1 M PB, and then embedded in Tissue-Tek O. C. T. Compound (Sakura Finetek Japan Co., Ltd.)/20% sucrose and frozen in liquid nitrogen.

(2) Measurement of Bispecific Anti-Human TfR (Cross-Reactivity with Monkey)/EphA4 Antibody Concentration

Measurement of concentrations of #12-aT04m02mv, #12-aT04m0201mv, #12-aT04m0205mv, and #12-aT04m0206mv in plasma and hippocampal tissue lysate collected according to the above-described method was performed according to the following steps. A 96-well plate was coated with a donkey anti-human IgG-Fcγ chain-specific F(ab′)₂ antibody (Jackson ImmunoResearch Inc.). After incubating for 1 hour at room temperature or overnight at 4° C., washing was performed with 0.02% Tween20/PBS three times. After blocking the wells at room temperature for one hour by 1% Block Ace (DS Pharma Biomedical Co., Ltd.), plasma or hippocampal tissue lysate diluted with 0.4% Block Ace was added and incubated at room temperature for 2 hours. After washing three times, a horseradish peroxidase-labeled goat anti-human IgG-Fcγ chain-specific F(ab′)2 antibody (Jackson ImmunoResearch Inc.) was added and incubated at room temperature for one hour. After washing three times, TMBZ (3,3′,5,5′-tetramethyl benzidine, SeraCare) solution was added to the well and incubated at room temperature for 15 minutes. An equivalent amount of quenching solution (2N sulfuric acid) was added to the well, and the absorbance at 450 nm and 650 nm was read by a microplate reader (Thermo).

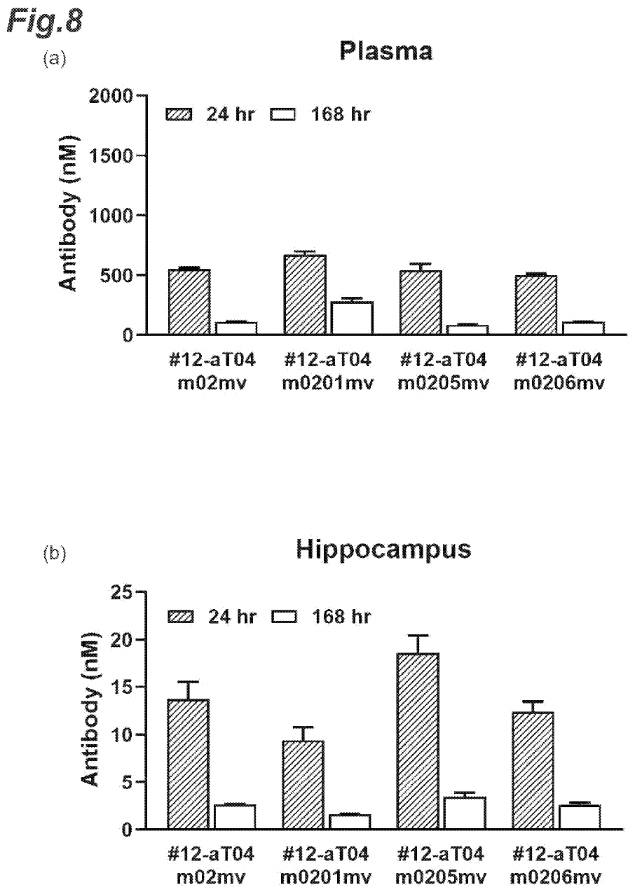

The concentrations calculated using the read absorbance are shown in FIG. 8. It was found from FIG. 8 that at 24 hours after antibody administration, the concentrations in hippocampus of individuals treated with #12-aT04m02mv, #12-aT04m0201mv, #12-aT04m0205mv, and #12-aT04m0206mv were higher than those of individuals treated with #12 shown in Experimental Example 5. Accordingly, it was found that these four bispecific anti-human TfR/EphA4 antibodies each have enhanced brain penetration capability.

(3) Intracerebral Distribution of Bispecific Anti-Human TfR (Cross-Reactivity with Monkey)/EphA4 Antibody

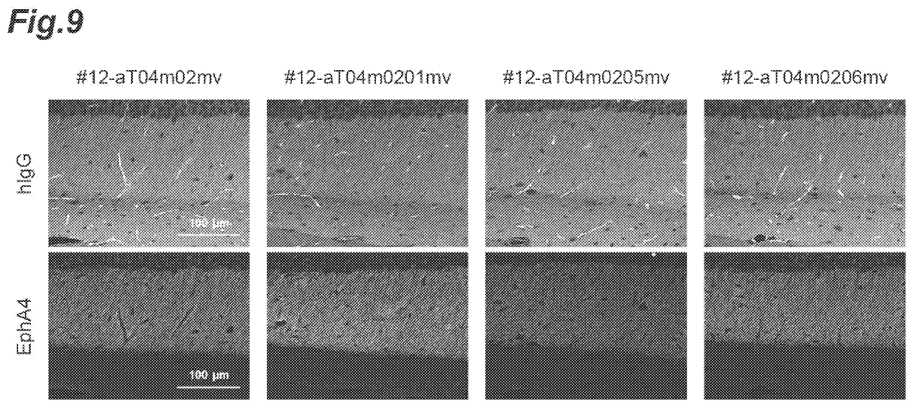

Confirmation of the intracerebral distributions of #12-aT04m02mv, #12-aT04m0201mv, #12-aT04m0205mv, and #12-aT04m0206mv was performed according to the following steps. The O. C. T. Compound-embedded frozen tissue prepared in (1) was sectioned in a thickness of 8 μM using Cryostat CM1860 (Leica), a section was mounted onto a glass slide, and then air dried in cold air, enclosed in a sealed bag and stored at −80° C. The glass slide for use in immunostaining was taken out from −80° C. and air dried in cold air, and then washed with PBS, immersed in 1% BSA/10% normal donkey serum (Jackson ImmunoResearch Inc.)/0.5% Triton (trademark) X-100/PBS solution, and subjected to blocking procedure for one hour. A goat anti-EphA4 antibody (R&D) and fluorescent-labeled donkey anti-human IgG antibody (Jackson ImmunoResearch Inc.), which were diluted in 1% BSA/3% normal donkey serum/0.5% Triton (trademark) X-100/PBS solution, were left to react overnight at 4° C. After washing with PBS three times and reacting with a fluorescent-labeled secondary antibody (Millipore) for one hour, the reacted tissue sections were washed again with PBS three times, Prolong Gold Antifade Reagent (Molecular probes) was placed on the section and sealed, and observed by LSM800 (Zeiss) to obtain images. The results are shown in FIG. 9.

The intracerebral distributions of the antibodies were evaluated in the SR layer of the hippocampus CA1 region where EphA4, the target molecule of #12, is highly expressed. As a result, higher signals of human IgG were observed in individuals treated with #12-aT04m02mv, #12-aT04m0201mv, #12-aT04m0205mv, and #12-aT04m0206mv (FIG. 9).

### Experimental Example 10: Production of Monoclonal Antibodies (aT04/aT04m02-Fab-His6)

### (1) Cloning of Fab Expression Vector

DNA sequences encoding the heavy chain variable region of aT04 (SEQ ID NO: 64) and the heavy chain variable region of aT04m02 (SEQ ID NO: 66) were inserted into a pcDNA3.4 vector in which a DNA sequence encoding CH1-His had been incorporated. Accordingly, vectors expressing the aT04-Fab-His6 gene (BR260; DNA sequence: SEQ ID NO: 80; amino acid sequence: SEQ ID NO: 81) and the aT04m02-Fab-His6 gene (BR261; DNA sequence: SEQ ID NO: 82; amino acid sequence: SEQ ID NO: 83) were constructed.

Table 9 shows the combinations of heavy and light chains that constitute each Fab.

TABLE 9

| | | | DNA/Amino | SEQ ID |
|---|---|---|---|---|
| ID | Antibody name | Antibody region | acid | NO: |
| BR260 | aT04-Fab-His6 | Heavy chain(H) | DNA | 80 |
| | H; aT04-VH-His6 | variable region | Amino acid | 81 |
| | L; aT04-VL | Light chain(L) | DNA | 30 |
| | | variable region | Amino acid | 31 |
| BR261 | aT04m02-Fab-His6 | Heavy chain(H) | DNA | 82 |
| | H; aT04m02-VH- | variable region | Amino acid | 83 |
| | His6 | Light chain(L) | DNA | 30 |
| | L; aT04-VL | variable region | Amino acid | 31 |

(2) Preparation of aT04/aT04m02-Fab-His6

BR260 and BR261 were transiently produced according to a manufacturer's standard protocol using an Expi293 expression system (Thermo). The cell culture medium containing the secreted Fab was collected and clarified by removing the cells by centrifugation. The clarified supernatant was purified using TALON Metal Affinity Resin (Clontech Laboratories, Inc.). The purified product was fractionated by a desalting column (Thermo) or size-exclusion chromatography (Superdex200 Increase 10/300 GL, Cytiva, Inc.) and substituted with 20 mM HEPES/100 mM NaCl (pH 7.5).

### (3) Construction of His-Tag-Fused Human TfR Extracellular Region Gene Expression Vector

The DNA sequence encoding a protein in which a polyhistidine tag (His-tag) is fused to the N-terminus of the human TfR extracellular region (positions 121 to 760) (referred to as “His-human TfR protein”) was amplified by PCR using the expression vector of SEQ ID NO: 16 as a template and inserted into a pcDNA3.4 vector. Accordingly, a vector expressing the His-human TfR protein gene (DNA sequence: SEQ ID NO: 84; amino acid sequence: SEQ ID NO: 85) was constructed.

### (4) Preparation of His-Human TfR Proteins

The His-human TfR protein was produced and purified with the following procedure using an Expi293 expression system (Thermo). The expression vector for the His-human TfR protein gene was transfected into Expi293F cells, and after four days, the culture medium was collected and clarified by removing the cells. The clarified supernatant was purified using TALON Metal Affinity Resin (Clontech). The purified product was buffer-exchanged with 20 mM HEPES/100 mM NaCl (pH 7.5) with a desalting column (Thermo), and concentrated by a centrifugal ultrafiltration filter.

### (5) Confirmation of Binding to Antigen (Human TfR)

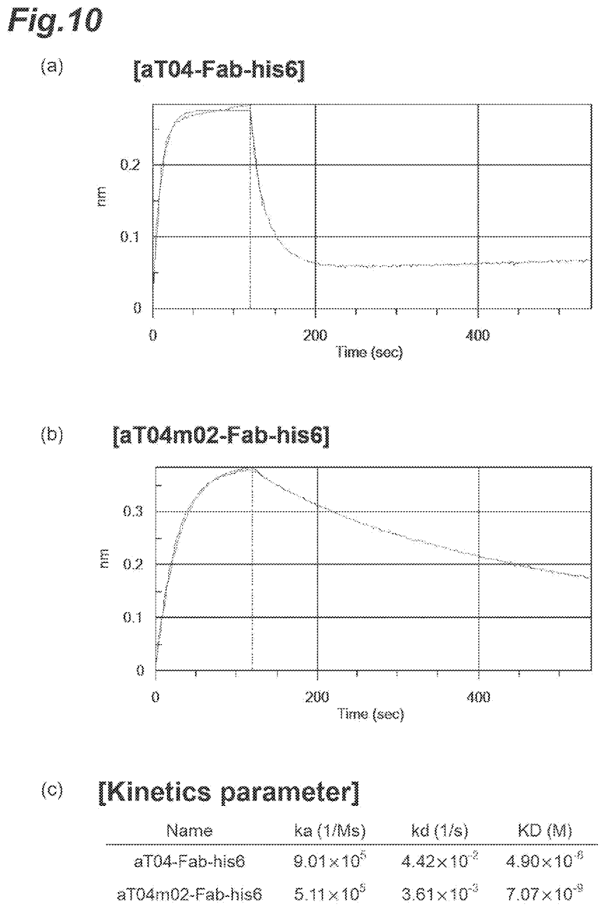

The binding affinity of BR260 and BR261 to human TfR was evaluated by Biolayer Interferometry (BLI) using Octet HTX system (Sartorius AG). His-human TfR diluted with acetate buffer was immobilized on AR2G Biosensors (Sartorius AG). Immobilization was performed by a standard amine coupling method according to the vendor's manual. The biosensors on which human TfR had been immobilized were immersed in wells containing BR260 and BR261 prepared in various dilution series, and the binding reactions were monitored. Waveform analysis was performed to calculate the binding affinity (KD value) of each antibody. The binding response curves and KD values are shown in FIG. 10. From FIG. 10, it was suggested that the binding affinity of BR261 (aT04m02-Fab-His6) to human TfR is approximately seven-fold stronger in KD value (lower KD value) than that of BR260 (aT04-Fab-His6).

### Experimental Example 11: Structural Analysis of Fab-hTfR-Holo Tf Complex (aT04/aT04m02)

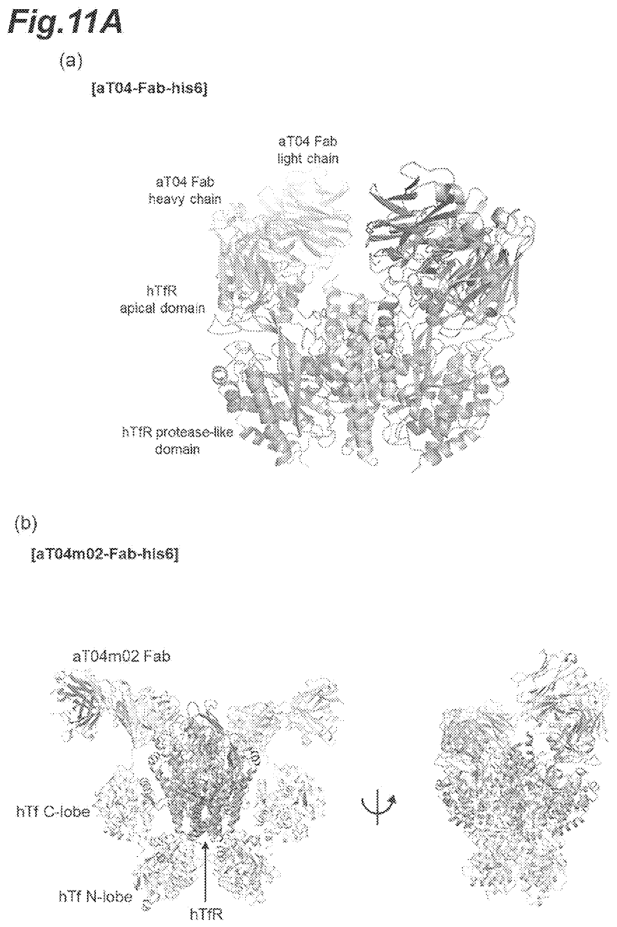

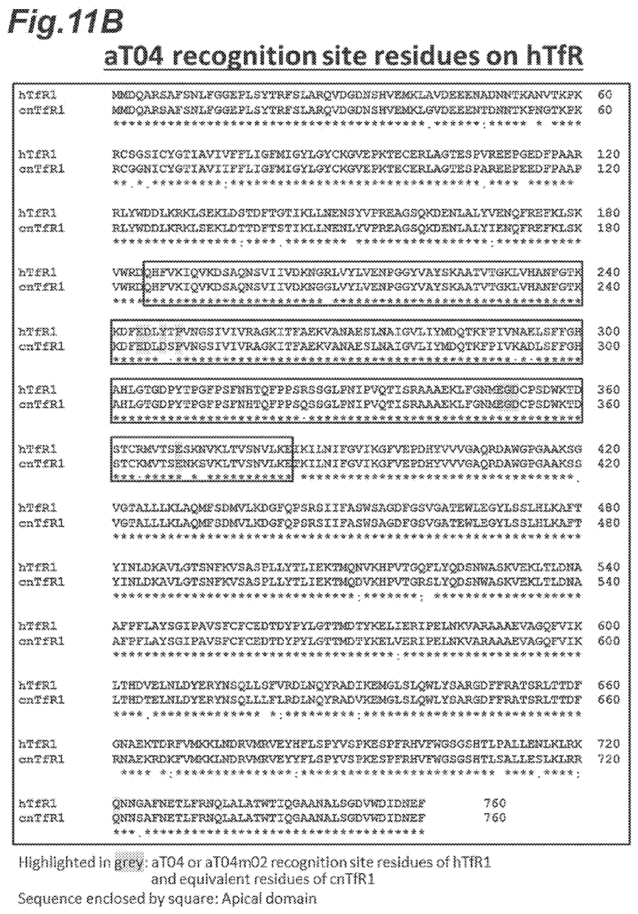

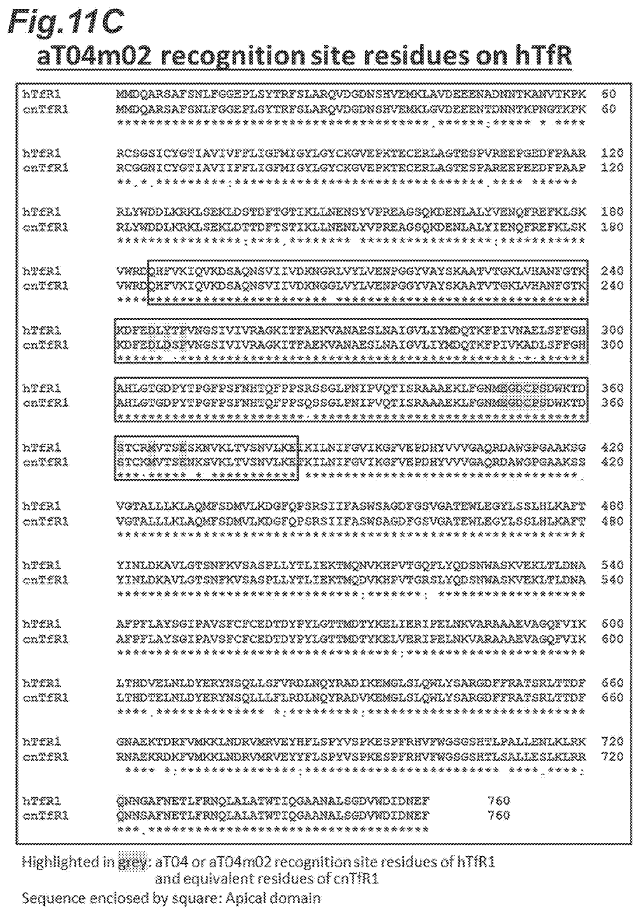

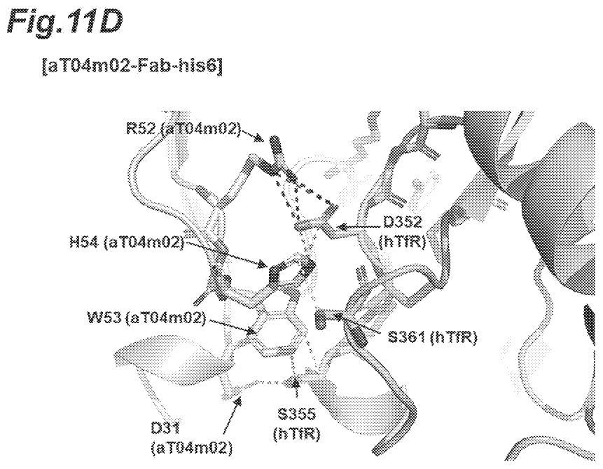

Structural analysis of BR260 (aT04-Fab-His6) and BR261 (aT04m02-Fab-His6) prepared in Experimental Example 10 in complex with human transferrin receptor (hTfR) was performed. aT04-Fab-His6 and the hTfR extracellular region were mixed at a molar ratio of 1:1 and incubated at 4° C. for one hour, and the complex was purified by size-exclusion chromatography using a Superdex200 column (Cytiva, Inc.). 7.7 mg/mL of hTfR/aT04-Fab-His6 complex was mixed with a well solution containing HEPES (pH 7.5), PEG200, and Silver Bullet-H1 to crystallize at 4° C. by the vapor diffusion method. Diffraction data was collected at a synchrotron facility and analyzed by the molecular replacement method. As a result, the crystal structure was determined at about 2.97 Å resolution as shown in FIG. 11A(a).

aT04m02-Fab-His6, the hTfR extracellular region, and holo human transferrin (R&D Systems, Inc.) (holoTf) were mixed at a molar ratio of 1.5:1:1 and incubated on ice for one hour, and the complex was fractionated by size-exclusion chromatography using a Superdex200 column. The fraction was concentrated with Amicon Ultra (Millipore) to obtain a complex solution at a concentration of about 5.9 mg/mL. The solution was applied to an electron microscope grid, the excess liquid was removed by filter paper, and then the grid was rapidly frozen in liquid ethane and analyzed by a cryo-electron microscope. The obtained image dataset was processed by the single-particle analysis method to obtain the structure at about 2.3 Å resolution as shown in FIG. 11A(b). There were two each of Fab, hTfR, holo Tf in a single complex molecule.

From the structures shown in FIG. 11A, the epitopes of each Fab and their spatial molecular recognition mechanism were revealed. The results of analyzing the binding interfaces between hTfR and aT04 or aT04m02-Fab are shown in FIG. 11B (aT04-Fab) and FIG. 11C (aT04m02-Fab), and the combinations of the interacting amino acids are shown in Table 10. From FIGS. 11B, 11C, and Table 10, it was found that both Fabs mainly recognize the apical domain of hTfR. Additionally, the structures of the aT04m02-Fab-His6 sites that were bound to human TfR are shown in FIG. 11D. Compared to aT04-Fab, the HCDR2 region of aT04m02-Fab that is defined by the Kabat numbering interacted directly with residues conserved between human TfR (SEQ ID NO: 256) and monkey TfR (SEQ ID NO: 257) (Asp352, Ser355, and Ser361 (D352, S355, and S361)), suggesting that this provides the species cross-reactivity of aT04m02 (FIG. 11D). On the other hand, direct interaction between aT04m02-Fab and holo Tf in the complex structure was not observed. It was found that the epitopes of aT04 are Glu244, Asp245, Tyr247, Pro249, Glu350, Gly351, Asp352, Glu369, and Gln721 (E244, D245, Y247, P249, E350, G351, D352, E369, and Q721) of human TfR, and the epitopes of aT04m02 are Asp245, Tyr247, Pro249, Glu350, Gly351, Asp352, Cys353, Pro354, Ser355, Ser361, Met365, Glu369, and Gln721 (D245, Y247, P249, E350, G351, D352, C353, P354, S355, S361, M365, E369, and Q721) of human TfR.

TABLE 10

| | Human TfR |
|---|---|
| aT04 heavy chain | |
| Lys100 | Asp245 |
| Ser101 | Asp352 |
| Arg103 | Tyr247 |
| Arg103 | Glu350 |
| Arg103 | Gly351 |
| His104 | Asp245 |
| His104 | Tyr247 |
| Trp105 | Asp245 |
| aT04 light chain | |
| Ser29 | Glu369 |
| Tyr30 | Tyr247 |
| Tyr31 | Tyr247 |
| Tyr31 | Pro249 |
| Asn52 | Glu244 |
| Arg53 | Gln721 |
| Tyr95 | Glu350 |
| aT04m02 heavy chain | |
| Asp31 | Ser355 |
| Arg52 | Asp352 |
| Trp53 | Cys353 |
| Trp53 | Ser355 |
| His54 | Asp352 |
| His54 | Ser361 |
| Arg103 | Glu350 |
| Arg103 | Gly351 |
| His104 | Tyr247 |
| His104 | Pro354 |
| His104 | Met365 |
| aT04m02 light chain | |
| Ser29 | Pro249 |
| Tyr30 | Tyr247 |
| Tyr31 | Tyr247 |
| Tyr31 | Pro249 |
| Tyr48 | Asp245 |
| Arg53 | Asn721 |
| Ser92 | Glu369 |
| Ser93 | Glu369 |

### Experimental Example 12: Establishment of Bispecific Anti-Human TfR/Aβ Antibody

### (1) Construction of Bispecific Anti-Human TfR/Aβ Human IgG1 Antibody and Mouse IgG2a Chimeric Antibody Gene Expression Vector

Similarly to Experimental Example 1, the knob-into-hole heterodimerization technique was applied to human IgG1 containing the variable regions of an anti-Aβ antibody (hereinafter referred to as “Ab1”), and a DNA sequence of a bispecific anti-human TfR/Aβ human IgG1 antibody (BR118), in which an aT04-scFv sequence was inserted at the C-terminus of the second heavy chain via a G4S linker sequence, was constructed. Accordingly, vectors expressing the first heavy chain gene (DNA sequence: SEQ ID NO: 86; amino acid sequence: SEQ ID NO: 87), the second heavy chain and scFv gene (DNA sequence: SEQ ID NO: 88; amino acid sequence: SEQ ID NO: 89, in the constant region and scFv region), and the light chain gene (DNA sequence: SEQ ID NO: 90; amino acid sequence: SEQ ID NO: 91, in the constant region) of BR118 were constructed. Next, in the vector expressing the second heavy chain and scFv genes, the DNA sequence encoding aT04-scFv was replaced with a DNA sequence encoding aT04m02-scFv. Accordingly, a vector expressing the second heavy chain and scFv genes (DNA sequence: SEQ ID NO: 92; amino acid sequence: SEQ ID NO: 93, in the constant region and scFv region) of the bispecific anti-human TfR/Aβ human IgG1 antibody (BR24), which exhibits cross-reactivity to human and monkey TfR, was constructed. Note that the light chain constant region of the anti-Aβ antibody Ab1 (BR15) is identical to SEQ ID NO: 91, and the heavy chain constant region consists of the amino acid sequence of SEQ ID NO: 95, encoded by the DNA sequence of SEQ ID NO: 94.

DNA sequences encoding (i) a first heavy chain containing the heavy chain variable region of another anti-Aβ antibody, DI 8 (A17D/R79T_DI 8 antibody disclosed in Patent Literature 4; hereinafter “DI 8”), and the heavy chain constant region of mouse IgG2a to which hole substitutions (Y349C, T366S, M368A, and Y407V) were introduced (DNA sequence: SEQ ID NO: 96; amino acid sequence: SEQ ID NO: 97); (ii) a second heavy chain containing the heavy chain variable region of DI 8, the heavy chain constant region of mouse IgG2a to which knob substitutions (P354C and T366W) were introduced, and an aT04-scFv fused at the C-terminus via a G4S linker (DNA sequence: SEQ ID NO: 98; amino acid sequence: SEQ ID NO: 99); and (iii) a light chain containing the light chain variable region of DI 8 and the light chain constant region of mouse Igκ (DNA sequence: SEQ ID NO: 100; amino acid sequence: SEQ ID NO: 101) were each synthesized de novo and cloned into a pcDNA3.4 vector, thereby constructing an expression vector for the bispecific anti-human TfR/Aβ mouse IgG2a chimeric antibody gene (BR282). Hereinafter, “Hk” in the antibody names in the tables indicates that the knob substitution is applied to the second heavy chain, and “mv” indicates that the scFv is fused at only one position. Note that the light chain of DI 8-mIgG2a (BR11) is identical to SEQ ID NO: 101, and the heavy chain consists of the amino acid sequence of SEQ ID NO: 103, encoded by the DNA sequence of SEQ ID NO: 102. The DNA sequence encoding the heavy chain of BR11 was inserted into a pEE6.4 vector, and the DNA sequence encoding the light chain was inserted into a pEE12.4 vector. Both vectors were then fused to construct an expression vector used for establishing a stable cell line of BR11.

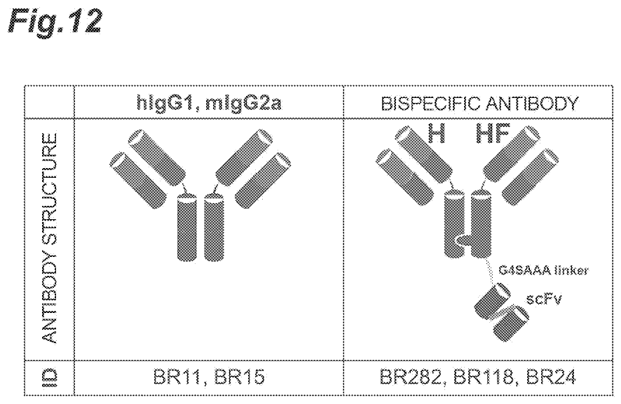

FIG. 12 shows schematic diagrams of the respective antibodies, and Table 11 shows the combinations of the first and second heavy chains and light chains included in each antibody. Note that, hereinafter, specific sequences and SEQ ID NOs for the variable region of Ab1 are not shown; however, the variable region of Ab1 is included in the Ab1 antibody produced and used in each Experimental Example, as well as in the bispecific antibodies containing Ab1.

TABLE 11

| | | | DNA/ | SEQ |
|---|---|---|---|---|
| ID | Antibody name | Antibody region | Amino acid | ID NO: |
| BR118 | Ab1-hIgG1-Hk- | First heavy chain | DNA | 86 |
| | aT04.1_mv | (H) | Amino acid | 87 |
| | H; hole (Y349C, | constant region | | |
| | T366S, L368A, | Second heavy | DNA | 88 |
| | Y407V) | chain (HF) | Amino acid | 89 |
| | HF; knob (S354C, | constant region | | |
| | T366W), linker | and scFv region | | |
| | (G4SAAA), aT04- | Light chain (L) | DNA | 90 |
| | scFv L; wt | constant region | Amino acid | 91 |
| BR24 | Ab1-hIgG1-Hk- | First heavy chain | DNA | 86 |
| | aT04m02.1_mv | (H) | Amino acid | 87 |
| | H; hole (Y349C, | constant region | | |
| | T366S, L368A, | Second heavy | DNA | 92 |
| | Y407V) | chain (HF) | Amino acid | 93 |
| | HF; knob (S354C, | constant region | | |
| | T366W), linker | and scFv region | | |
| | (G4SAAA), | Light chain (L) | DNA | 90 |
| | aT04m02-scFv | constant region | Amino acid | 91 |
| | L; wt | | | |
| BR15 | Ab1-hIgG1 | Heavy chain (H) | DNA | 94 |
| | | constant region | Amino acid | 95 |
| | | Light chain (L) | DNA | 90 |
| | | constant region | Amino acid | 91 |
| BR282 | DI 8-mIgG2a-Hk- | First heavy chain | DNA | 96 |
| | aT04.1_mv | (H) | Amino acid | 97 |
| | H; hole (Y349C, | Second heavy | DNA | 98 |
| | T366S, M368A, | chain (HF) and | Amino acid | 99 |
| | Y407V) | scFv region | | |
| | HF; knob (P354C, | Light chain (L) | DNA | 100 |
| | T366W), linker | | Amino acid | 101 |
| | (G4SAAA), aT04- | | | |
| | scFv L; wt | | | |
| BR11 | DI 8-mIgG2a | Heavy chain (H) | DNA | 102 |
| | | | Amino acid | 103 |
| | | Light chain (L) | DNA | 100 |
| | | | Amino acid | 101 |

In addition, the amino acid sequences of the CDRs of the heavy and light chains of DI 8 according to the Kabat definition, as well as the amino acid sequences of the heavy chain variable regions (VH) and light chain variable regions (VL), are shown in Tables 12 and 13 below.

TABLE 12
Amino acid sequences of CDRs of DI 8

| | | SEQ ID |
|---|---|---|
| Name | Sequence | NO |
| HCDR1 | SFGMH | 232 |
| HCDR2 | YISSGSSTIYYGDTVKG | 233 |
| HCDR3 | EGGYYYGRSYYTMDY | 234 |
| LCDR1 | RSSQSIVHSNGNTYLE | 235 |
| LCDR2 | KVSNRFS | 236 |
| LCDR3 | FQGSHVPPT | 237 |

TABLE 13
Amino acid sequences of heavy and
light chain variable regions of DI 8

| | SEQ | |
|---|---|---|
| | ID | |
| Name | NO | Sequence |
| DI 8- | 238 | EVOLVESGGGLVQPGGSLRLSCSASGFTFSSFGMH |
| VH | | WVRQTPGKGLEWVAYISSGSSTIYYGDTVKGRFTI |
| | | SRDNAKNSLFLQMSSLRAEDTAVYYCAREGGYY |
| | | YGRSYYTMDYWGQGTTVTVSS |
| DI 8- | 239 | DVVMTQSPLSLPATPGDPASISCRSSQSIVHSNGN |
| VL | | TYLEWYLQKPGQSPKLLIYKVSNRFSGVPDRFSGS |
| | | GSGTDFTLTISRVDAEDVGIYYCFQGSHVPPTFGP |
| | | GTKLEIK |

### (2) Production and Purification of Bispecific Anti-Human TfR/Aβ Human IgG1 Antibody and Mouse IgG2a Chimeric Antibody

BR15, BR118, and BR24 were transiently produced according to the manufacturer's standard protocol using an Expi293 expression system (Thermo). The mixing ratio of the vectors expressing the first heavy chain gene, the second heavy chain gene, and the light chain gene of each bispecific anti-human TfR/Aβ human IgG1 antibody was optimized individually. The cell culture medium containing the secreted antibody was collected 6 to 7 days after transfection. BR282 was transiently produced according to the manufacturer's high-titer protocol using an ExpiCHO expression system (Thermo), and the cell culture medium containing the antibody was collected 11 days after transfection. For BR11, the stable cell line prepared by using a GS system (Lonza) was cultured for 13 days, and the cell culture medium containing the antibody was collected. Each cell culture supernatant was clarified by centrifugation to remove the cells. The clarified supernatants were purified using a HiTrap MabSelect SuRe column (Cytiva, Inc.) or a KanCapA3G resin (KANEKA CORPORATION). The resulting eluates were buffer-exchanged with PBS by dialysis or using a desalting column.

### Experimental Example 13: Binding Evaluation of Bispecific Anti-Human TfR/Aβ Human IgG1 and Mouse IgG2a Chimeric Antibody to Human TfR and Protofibril-Like Aβ

### (1) Preparation of TfR Proteins

The His-MBP-human TfR protein including the amino acid sequence corresponding to the extracellular region of human TfR (positions 101 to 760) (SEQ ID NO: 16) was produced and purified with the procedure described in Experimental Example 1, using an Expi293 expression system (Thermo).

### (2) Binding Evaluation to Human TfR

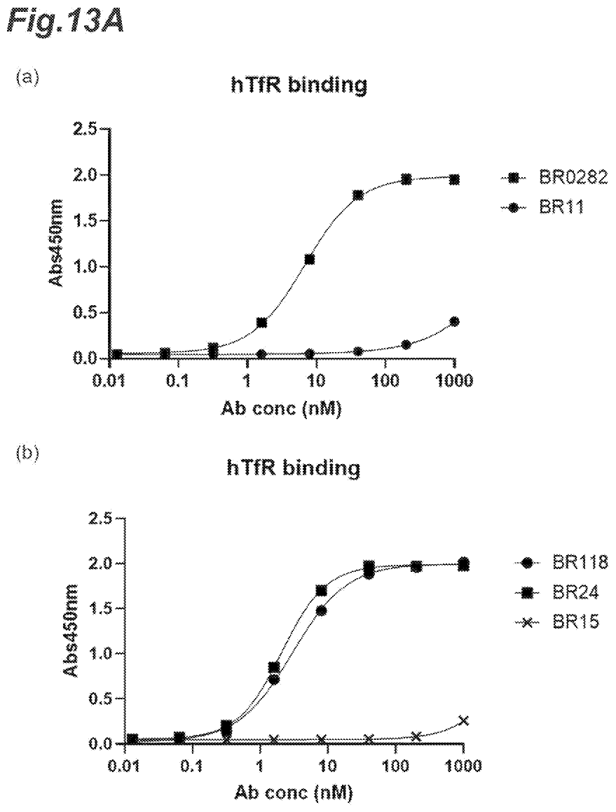

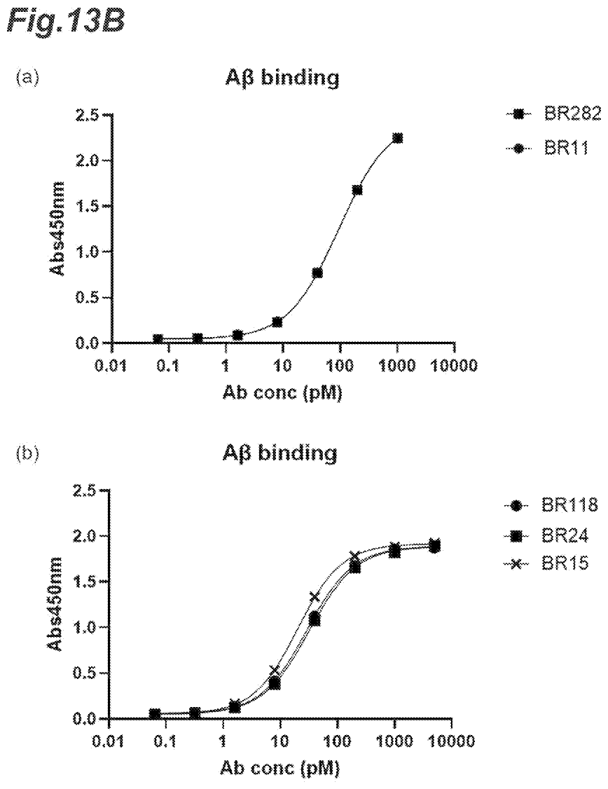

The binding of BR15, BR118, BR24, BR11, and BR282 to human TfR was evaluated by ELISA as follows. Ni-NTA HisSorb Plates (QIAGEN) or Nickel Coated Plates (Thermo) were washed once with 0.02% Tween20/PBS, and a His-MBP-human TfR protein was added and incubated overnight at 4° C. or for two hours at room temperature to immobilize the His-MBP-human TfR protein on the plate. After washing the plate three times with 0.02% Tween20/PBS, the antibody to be evaluated was added to each well and incubated at room temperature for one to two hours. After washing three times with 0.02% Tween20/PBS, the amount of antibody bound to human TfR was detected using an HRP-labeled anti-human IgG antibody (Bethyl Laboratories, Inc.) or an HRP-labeled anti-mouse IgG antibody (Jackson ImmunoResearch Inc.). For a chromogenic substrate, TMBZ (3,3′,5,5′-tetramethyl benzidine, SeraCare) solution was used, and the reaction was quenched by adding an equivalent amount of 1N sulfuric acid. The graphs of the absorbance of each antibody concentration at 450 nm are shown in FIG. 13A. As shown in FIG. 13A, all the bispecific antibodies exhibited equivalent binding properties to human TfR.

### (3) Binding Evaluation to Protofibril-Like Aβ

Since it has been reported that immobilizing monomer Aβ 1-40 on an ELISA plate provides a protofibril-like structure (Stina Tucker et al. Journal of Alzheimer's Disease 43 (2015) 575-588), the binding of BR15, BR118, BR24, BR11, and BR282 to protofibril-like Aβ was evaluated by ELISA as follows. A solution of monomer Aβ 1-40 (Anaspec, Inc.) was added to MaxiSorp Plates (Thermo) and incubated overnight or longer at 4° C. to immobilize the monomer Aβ 1-40 on the plate. After removing the solution, 1% BSA/PBS was added for blocking at room temperature for one hour or longer. After washing the plate three times with 0.02% Tween20/PBS, the antibody to be evaluated was added to each well and incubated at room temperature for one hour. Subsequent steps were performed in the same manner as the ELISA for human TfR described above. The graphs of the absorbance of each antibody concentration at 450 nm are shown in FIG. 13B. As shown in FIG. 13B all the bispecific antibodies exhibited equivalent binding properties to those of naked antibodies (BR15 and BR11) which do not contain a human TfR antibody, to the immobilized monomer Aβ.

### Experimental Example 14: Evaluation of Dose-Reduction by Bispecific Anti-Human TfR/Aβ Mouse IgG2a Antibody

### (1) Establishment of APP KI/hTfR-KI Mice

APP KI mice in which amyloid plaque is formed in the brain (RBRC06344, Institute of Physical and Chemical Research) were crossed with hTfR-KI mice generated in Experimental Example 5. APP KI/hTfR-KI (hetero/hetero) mice obtained by crossing were further crossed with siblings to produce APP KI/hTfR-KI (homo/homo) mice. After production, the strain was maintained by sibling breeding.

(2) Collection of Samples from Mice Treated with a Bispecific Anti-Human TfR/Aβ Mouse IgG2a Antibody.

6-month-old male APP KI/hTfR-KI (homo/homo) mice were administered the anti-Aβ mouse IgG2a antibody BR11 at a dose of 10 mg/kg, and the bispecific anti-human TfR/Aβ mouse IgG2a antibody BR282 at doses of 0.1 mg/kg, 0.3 mg/kg, 1.0 mg/kg, and 3.0 mg/kg, calculated as an IgG equivalent amount by intraperitoneal injection (10 mL/kg) once a week for 16 weeks. In the control group, PBS was administered intraperitoneally. One week after the final administration, blood was collected under anesthesia, followed by exsanguination via incision of the right atrial appendage and transcardial perfusion with heparin-containing PBS. The brain was harvested and sectioned into left and right cerebral hemispheres. The left cerebral hemisphere was immersed in 1% paraformaldehyde (TAAB Laboratories Equipment Ltd.)/PBS and fixed at 4° C. for 24 hours. After fixation, the left cerebral hemisphere was sequentially replaced with 10% sucrose/PBS and 20% sucrose/PBS, and embedded in Tissue-Tek O. C. T. Compound (Leica), and frozen in liquid nitrogen to prepare a frozen blocks. The right cerebral hemisphere was weighed to determine wet tissue weight, frozen in liquid nitrogen, and stored at −80° C. The blood sample was centrifuged at 1,200×g for 15 minutes at 4° C., and the supernatant was collected as plasma.

### (3) Measurement of Antibody Concentration in Plasma

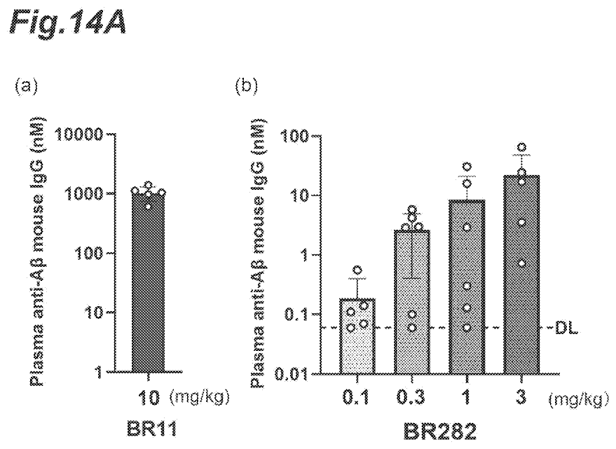

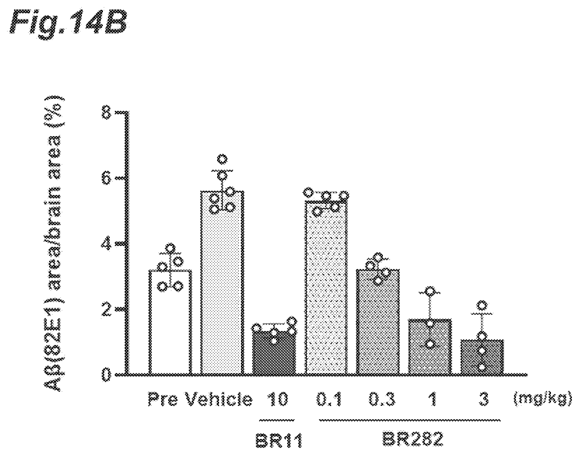

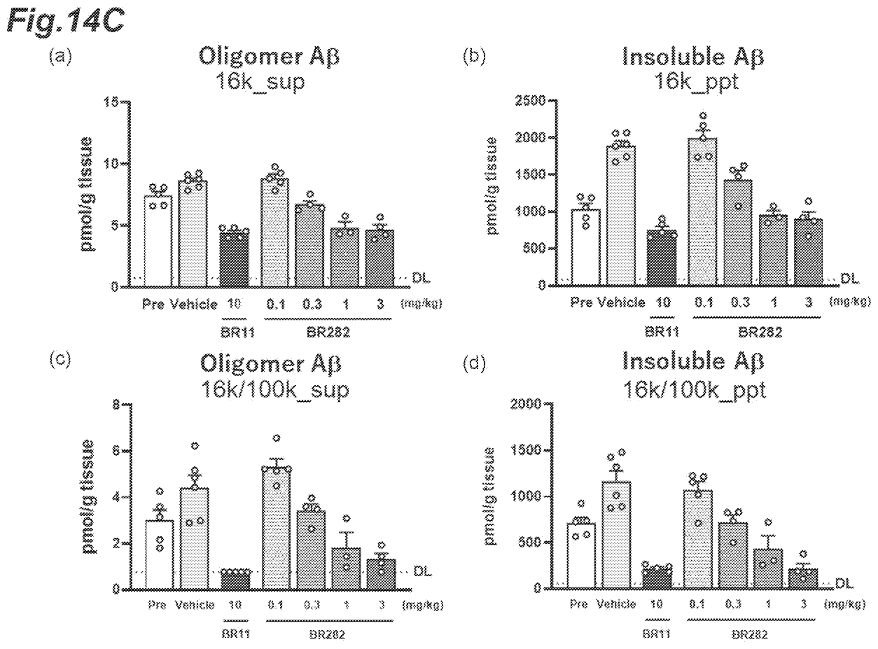

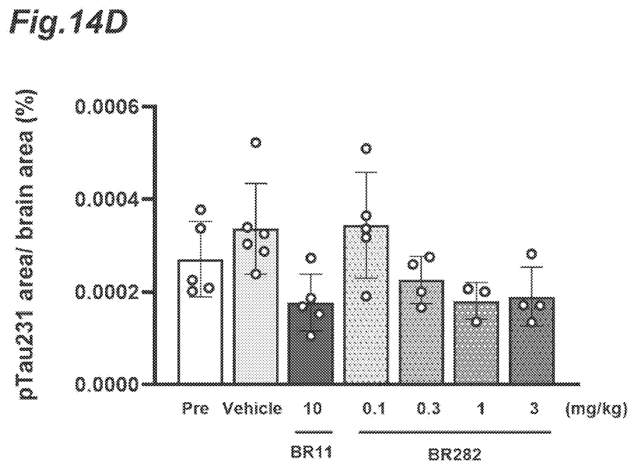

The measurement of plasma concentration of BR11 and BR282 was performed by ELISA as described below. A monomer Aβ 1-40 (Anaspec, Inc.) was added to MaxiSorp Plates (Thermo) and incubated overnight at 4° C. After blocking with 1% BSA/PBS at room temperature for one hour, the plates were washed with 0.02% Tween20/PBS three times and mouse plasma diluted with 0.1% BSA/0.02% Tween20/PBS was added to react at room temperature for one hour. After washing with 0.02% Tween20/PBS three times, an HRP-labeled goat anti-mouse IgG antibody (Jackson ImmunoResearch Inc.) was added and incubated at room temperature for one hour. After washing with 0.02% Tween20/PBS five times, TMB substrate solution (SeraCare) was added and incubated at room temperature for 10 minutes. An equivalent amount of 2N sulfuric acid was added to quench the reaction, and the absorbance at 450 nm was measured with a microplate reader SpectraMax (Molecular Devices, LLC). Calibration curves were created based on the absorbance of standard samples at each concentration, and the antibody concentrations of the plasma samples were determined (FIG. 14A). The detection limit was set to 0.06 nM, and individuals of less than 0.06 nM were shown as 0.06 nM. Individuals with markedly low antibody concentration in plasma samples were excluded from the following analysis. The threshold antibody concentrations set for each dosing group were as follows: 0.05 nM (0.1 mg/kg group), 0.15 nM (0.3 mg/kg group), 0.5 nM (1.0 mg/kg group), and 1.5 nM (3.0 mg/kg group).

### (4) Amyloid Plaque Quantification

Frozen brain sections (10 μm thickness) were prepared using a cryostat CM1860 (Leica) and mounted on a glass slide. After air drying, sections were treated with PBS and 0.1% Triton (trademark) X-100/PBS at room temperature for 5 minutes. After blocking with 0.1% Triton (trademark) X-100/1% normal donkey serum/1× Block Ace at room temperature for one hour, sections were incubated with Alexa555-labeled mouse anti-Amyloid antibody (82E1) at room temperature for two hours. After washing, mounting was performed with Prolong Glass Antifade Mountant (Thermo). Images were acquired using NanoZoomer (Hamamatsu Photonics) and amyloid plaque areas were quantified based on 82E1 signals using NIS Elements software (Nikon). The mean plaque area per brain area for each mouse was calculated. The results of amyloid plaque quantification are shown in FIG. 14B. Effects equivalent to the reducing effects of amyloid plaque which are observed by the administration of anti-Aβ antibody BR11 at 10 mg/kg were confirmed in the case of the bispecific anti-human TfR/Aβ antibody BR282 at a lower dose.

### (5) Biochemical Quantification of Aβ in Brain Tissue

To the right cerebral hemisphere collected in step (2), 10 times the amount of the brain wet weight of TBS/complete (Roche) was added and homogenized using a homogenizer (TAITEC Corporation), and then, centrifugation was performed at 16,000×g for 60 minutes at 4° C., and the supernatant after the centrifugation was referred to as 16k_sup, and the precipitate was referred to as 16k_ppt. In addition, 16k_sup was further ultracentrifuged at 100,000×g for 60 minutes at 4° C., and the supernatant after the centrifugation was referred to as 16k/100k_sup, and the precipitate was referred to as 16k/100k_ppt. For 16k_sup and 16k/100k_sup, the reaction was performed using Human Amyloidβ Oligomers (82E1-specific) Assay Kit (IBL Co., Ltd.) according to the instructions in the kit. After the reaction, the absorbance at 450 nm was measured with a microplate reader SpectraMax (Molecular Devices, LLC), and the concentration of oligomer Aβ was determined, and further the amount of oligomer Aβ per g of wet brain tissue was calculated. For 16k_ppt and 16k/100k_ppt, the reaction was performed using human Amyloid beta (1-42) kit (FUJIFILM Wako Pure) according to the instructions in the kit. After the reaction, the absorbance at 450 nm was measured with a microplate reader SpectraMax (Molecular Devices, LLC), and the concentration of insoluble Aβ was determined, and further the amount of insoluble Aβ per g of wet brain tissue was calculated. The results of the oligomer Aβ and insoluble Aβ are shown in FIG. 14C. For the amounts of oligomer Aβ (16k_sup and 16k/100k_sup), insoluble Aβ (16k_ppt), and insoluble Aβ (16k/100k_ppt), 0.76 μmol/g tissue, 89.43 pmol/g tissue, and 52.17 μmol/g tissue, respectively, were shown as the detection limits (DL). Individuals in which the concentration of oligomer Aβ was less than the detection limit were shown as 0.76 μmol/g tissue. Effects equivalent to the reducing effects of oligomer Aβ and insoluble Aβ which are observed by the administration of anti-Aβ antibody BR11 at 10 mg/kg were confirmed in the case of the bispecific anti-human TfR/Aβ antibody BR282 at a lower dose.

(6) Quantification of Phosphorylated tau231 Area

Frozen brain sections (10 μm thickness) were prepared using a cryostat CM1860 (Leica) and mounted on a glass slide. After air drying, sections were treated with PBS and 0.1% Triton (trademark) X-100/PBS at room temperature for 5 minutes. After blocking with 0.1% Triton (trademark) X-100/1% normal donkey serum/1× Block Ace at room temperature for one hour, sections were incubated with Alexa488-labeled mouse anti-phosphorylated tau231 antibody (AT180) at room temperature for two hours. After washing, mounting was performed with Prolong Glass Antifade Mountant (Thermo). Images were acquired using NanoZoomer (Hamamatsu Photonics) and the phosphorylated tau231 area was quantified using NIS Elements software (Nikon). The mean of phosphorylated tau231 area per brain area for each mouse was calculated, and the area of phosphorylated tau231 was quantified. The results of phosphorylated tau231 area are shown in FIG. 14D. Effects equivalent to the reducing effects of phosphorylated tau231 area which are observed by the administration of anti-Aβ antibody BR11 at 10 mg/kg were confirmed in the case of the bispecific anti-human TfR/Aβ antibody BR282 at a lower dose.

### Experimental Example 15: Evaluation of ARIA Risk

It has been reported that, by administering the anti-Aβ antibodies to mice having CAA, immune cells such as T cells infiltrate from the periphery into brain, which induces angiopathy involving BBB breakdown (Xavier Taylor et al. Mol Neurodegener. 2024 Oct. 21; 19(1): 77). Therefore, the number of CD45+ Iba1− cells that can distinguish T cells infiltrated into the brain and peripheral immune cells such as B cells and neutrophils was used as an evaluation index of ARIA risk.

(1) Establishment of 5xFAD/hTfR-KI Mice

5xFAD mice are genetically modified mice in which a human APP gene harboring the Sweden mutation (K670N/M671L), the Florida mutation (I716V), and the London mutation (V717I), as well as a human PSEN1 gene harboring M146L and L286V mutations identified in familial Alzheimer's disease. The strain exhibits age-dependent amyloid plaque accumulation, gliosis, and cognitive impairment (Holly Oakley et al. Journal of Neuroscience 4 Oct. 2006, 26 (40) 10129-10140). In 5xFAD mice, Aβ deposits not only in the brain parenchyma but also in blood vessels, exhibiting cerebral amyloid angiopathy (CAA) (Patrizia Giannoni et al. Neurobiology of Disease Volume 88, April 2016, Pages 107-117). 5xFAD mice (MMRRC034848, Jackson Laboratory, Inc.) were crossed with hTfR-KI mice generated in Experimental Example 5. 5xFAD/hTfR-KI mice obtained by crossing were further crossed with siblings using hemi/hetero and wild/hetero genotypes to generate 5xFAD/hTfR-KI (hemi/homo) mice. After generating, the strain was maintained by sibling reproduction.

(2) Sample Preparation from Animals Given Bispecific Anti-Human TfR/Aβ Mouse IgG2a Antibody

Male 5xFAD/hTfR-KI (hemi/homo) mice aged 13 months were administered intraperitoneally with 50 mg/kg of anti-Aβ mouse IgG2a antibody BR11, and with bispecific anti-human TfR/Aβ mouse IgG2a antibody BR282 at doses equivalent to 10 mg/kg or 30 mg/kg IgG, once weekly for 8 weeks (10 mL/kg). The control group received PBS intraperitoneally. One week after the final administration, blood was collected under anesthesia, exsanguination via right atrial appendage and transcardial perfusion with heparin-containing PBS were performed, and the brain was harvested. The brain was divided into left and right cerebral hemispheres. The blood was centrifuged at 1,200×g for 15 minutes 4° C., and the supernatant was collected as plasma. The right cerebral hemisphere was frozen in liquid nitrogen, and then, stored at −80° C. Ten volumes of TBS relative to wet tissue weight were added and tissue was homogenized using a homogenizer (BRANSON, Inc.), followed by centrifugation at 16,000×g for 60 minutes at 4° C. The supernatant after the centrifugation was collected as TBS soluble fraction. The left cerebral hemisphere was immersed and fixed for 24 hours at 4° C. in 1% paraformaldehyde (TAAB Laboratories Equipment Ltd.)/PBS. It was substituted with 10% sucrose/PBS and 20% sucrose/PBS, and then, embedded in Tissue-Tek O. C. T. Compound (Leica) and frozen in liquid nitrogen.

### (3) Measurement of Plasma Concentration of Bispecific Anti-Human TfR/Aβ Mouse IgG2a Antibody

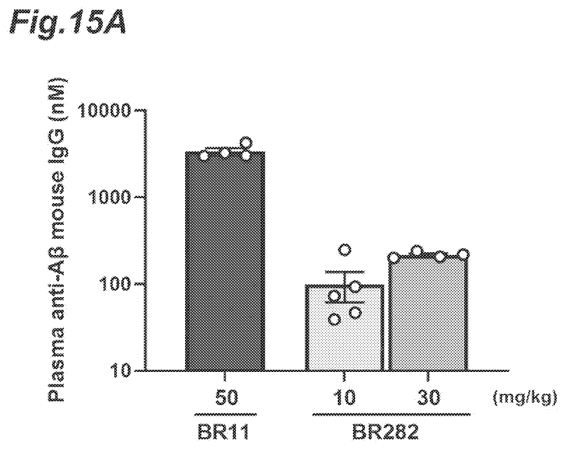

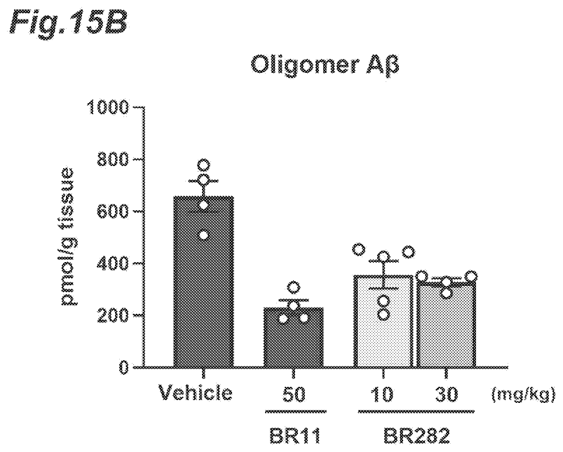

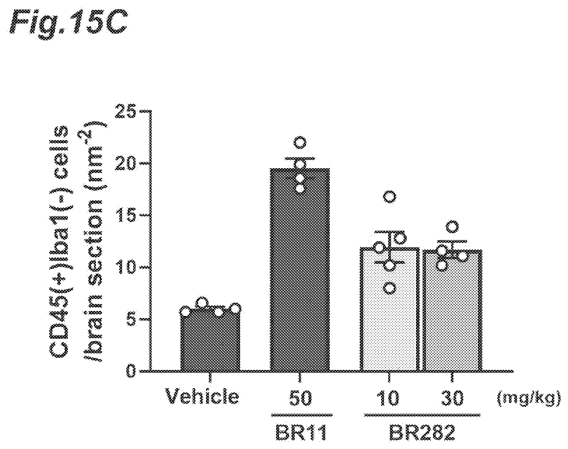

The measurement of plasma concentration of BR11 and BR282 was performed by ELISA as described below. A monomer Aβ 1-40 (Anaspec, Inc.) was added to MaxiSorp Plates (Thermo) and incubated overnight at 4° C. After blocking with 1% BSA/PBS at room temperature for one hour, the plates were washed with 0.02% Tween20/PBS three times and plasma diluted with 0.1% BSA/0.02% Tween20/PBS was added to react at room temperature for one hour. After washing with 0.02% Tween20/PBS three times, an HRP-labeled goat anti-mouse IgG antibody (Jackson ImmunoResearch Inc.) was added and incubated at room temperature for one hour. After washing with 0.02% Tween20/PBS five times, TMB substrate solution (SeraCare) was added and incubated at room temperature for 10 minutes. An equivalent amount of 2N sulfuric acid was added to quench the reaction, and the absorbance at 450 nm was measured with a microplate reader SpectraMax (Molecular Devices, LLC). Calibration curves were created based on the absorbance of standard samples at each concentration, and the antibody concentrations of the plasma samples were determined. The results are shown in FIG. 15A. In each group to which BR11 and BR282 were administered, it was confirmed that no individuals whose antibody concentration in plasma was markedly low were observed.

### (4) Amount of Oligomer Aβ in TBS Soluble Fraction

The reaction was performed using Human Amyloidβ Oligomers (82E1-specific) Assay Kit (IBL Co., Ltd.) according to the instructions in the kit. After the reaction, the absorbance at 450 nm was measured with a microplate reader SpectraMax (Molecular Devices, LLC). The concentration of oligomer Aβ included in the TBS soluble fraction was determined, and the amount of oligomer Aβ per g of wet brain tissue was calculated. The results are shown in FIG. 15B. It was confirmed that the amounts of TBS soluble oligomer Aβ were reduced compared to the control group, by the administration of BR11 at 50 mg/kg, and the administration of BR282 at 10 mg/kg or 30 mg/kg.

### (5) Evaluation of ARIA Risk Using Immune Cell Infiltration as Index

The prepared O. C. T. Compound-embedded and frozen tissue was sectioned in a thickness of 10 μm using Cryostat CM1860 (Leica), a section was mounted on a glass slide, and then air dried in cold air, enclosed in a sealed bag and stored at −80° C. The section on the glass slide was washed with PBS, immersed in 1% normal donkey serum (Jackson ImmunoResearch Inc.)/0.1% Triton (trademark) X-100/25% Block Ace solution, and subjected to blocking procedure at room temperature for one hour. CD45 antibody (R&D Systems, Inc.) and Iba1 antibody (FUJIFILM Wako Pure) diluted in PBS containing 0.1% normal donkey serum (Jackson ImmunoResearch Inc.)/0.1% Triton (trademark) X-100/2.5% Block Ace solution were incubated at room temperature for two hours. After performing washing procedure three times with 0.1% Triton (trademark) X-100/PBS solution for 5 minutes, a fluorescent-labeled secondary antibody (Jackson ImmunoResearch Inc.) was incubated at room temperature for one hour, and then, cell nucleus staining (Life Technologies Hoechst 33342) was incubated at room temperature for 5 minutes. After performing washing procedure three times with 0.1% Triton (trademark) X-100/PBS solution for 5 minutes, the mixture was washed with PBS once, and then, Prolong Gold antifade reagent (Thermo) was placed on the section and sealed, and observed by Nanozoomer S60 (Hamamatsu Photonics K.K.) to obtain images. The number of infiltrating CD45+ Iba1− peripheral immune cells in the brain tissue was quantified and analyzed using NIS elements (Nikon Corporation). For the obtained data, one-way analysis of variance (one-way ANOVA) was performed, followed by Tukey's multiple comparison test to compare with the control group. As a result, it was found that the bispecific anti-human TfR/Aβ antibody BR282 had a statistically significantly reduced number of infiltrating peripheral immune cells, compared to the anti-Aβ antibody BR11 (FIG. 15C).

### Experimental Example 16: Reticulocyte Depletion

(1) Establishment of hTfR-ApD-KI Mice

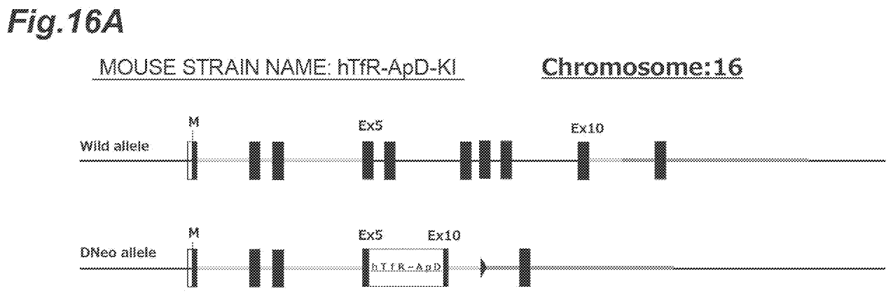

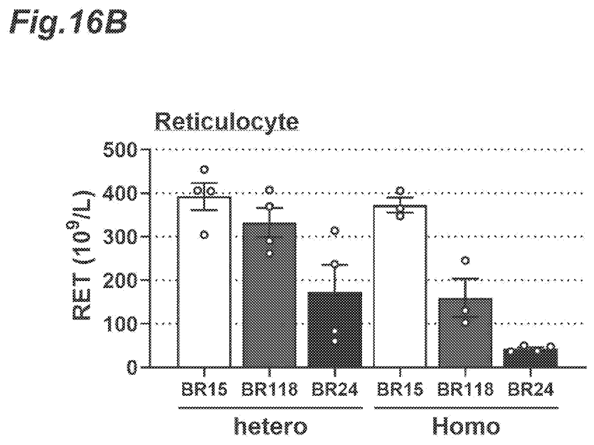

To generate hTfR-ApD-KI mice, a method for producing aggregated embryos using mouse 8-cell stage embryos and genetically engineered ES cells, was used. Aggregated embryos were prepared using MCH (ICR) mouse-derived 8-cell stage embryos and ES cells in which the mouse Tfrc genes (Exon 5 to Exon 10), corresponding to the apical domain (ApD), were replaced with the corresponding human TFRC gene ApD by homologous recombination, and the embryo were transferred into pseudopregnant female mice, and thereby a human TfR-ApD-KI mouse strain was established. Specifically, a targeting vector was produced, and it was introduced into ES cells using an electroporation method. As a result, homologous recombination between Exon 5 to Exon 10 in the mouse Tfrc gene and a targeting vector resulted in replacement with the human TFRC ApD sequence, thereby generating recombinant ES cells were produced (FIG. 16A). Subsequently, by embryo aggregation method using recombinant ES cells and MCH (ICR) mouse-derived 8-cell stage embryos, chimeric embryos into which ES cells were introduced were generated, and the chimeric embryos were transferred into pseudopregnant female mice. Male chimeric mice obtained by embryo transfer were crossed with wild-type female (C57BL/6J) to generate F1 generation heterozygous mice. F1 heterozygous male mice were back-crossed with wild-type female again to generate N2 generation heterozygous mice, and the strain was established. By sib crossing of established heterozygous mice, homozygous mice hTfR-ApD-KI mice were generated.

(2) Sample Preparation from Bispecific Anti-Human TfR/Aβ Antibody Treated Animals

To heterozygous mice (hetero) and homozygous mice (homo) generated by the method in (1) above, the anti-Aβ human IgG1 antibody BR15, the bispecific anti-human TfR/Aβ human IgG1 antibody BR118 or BR24 were administered via the tail vein at a dose of 20 mg/kg (10 mL/kg), and 24 hours after the administration, blood was collected using heparin.

### (3) Blood Test

Hematology test was performed using the blood samples of the above (2). The results are shown in FIG. 16B. As a noticeable change, there was a tendency toward lower reticulocyte (RET) values in BR118 and BR24, and its degree was greater in BR24 than BR118. In addition, the lowering tendency was greater in homo than hetero.

### Experimental Example 17: Production of Bispecific Human IgG1 Antibody with Fc-Mediated Effector Function Suppressed

(1) Construction of Bispecific Anti-Human TfR/Aβ Human IgG1 Antibody Gene Expression Vector with Fc-Mediated Effector Function Suppressed

The knob-into-hole heterodimerization technique was applied to human IgG1 containing the variable regions of a humanized anti-Aβ antibody Ab1 (humanized Ab1; hereinafter “HuAb1”). An aT04m02-scFv sequence was inserted at the C-terminus of the second heavy chain via a G4S linker sequence, and a sequence in which asparagine at residue 297 according to EU numbering was substituted with alanine (N297A) was synthesized. Accordingly, vectors expressing the first heavy chain gene (DNA sequence: SEQ ID NO: 104; amino acid sequence: SEQ ID NO: 105, in the constant region) and the second heavy chain and scFv gene (DNA sequence: SEQ ID NO: 106; amino acid sequence: SEQ ID NO: 107, in the constant region and scFv region) were constructed (BR218). Hereinafter, “N297A” in the antibody names in the tables refers to antibodies containing the N297A substitution in both the first and second heavy chains. Similarly, in the vector expressing the second heavy chain containing the constant region of SEQ ID NO: 107, a sequence in which leucine at residues 234 and 235 according to EU numbering was substituted with alanine (LALA), instead of N297A, was synthesized (DNA sequence: SEQ ID NO: 108; amino acid sequence: SEQ ID NO: 109, in the constant region and scFv region) and cloned into a pcDNA3.4 vector (BR189). Hereinafter, “cisLALA” refers to antibodies containing the LALA substitution in the second heavy chain. Furthermore, in the vector expressing the first heavy chain containing the amino acid sequence of SEQ ID NO: 105 and the second heavy chain and scFv containing the amino acid sequence of SEQ ID NO: 107, the N297A substitution was replaced with substitutions in which lysine at residues 147 and 213 according to EU numbering was replaced with glutamic acid (K147E and K213E), and the DNA sequence encoding aT04m02-scFv was replaced with a DNA sequence encoding aT04m02-scFab. Accordingly, the first heavy chain gene (DNA sequence: SEQ ID NO: 122; amino acid sequence: SEQ ID NO: 123, in the constant region) and the second heavy chain and scFab gene (DNA sequence: SEQ ID NO: 110; amino acid sequence: SEQ ID NO: 111, in the constant region and scFab region) were synthesized and cloned into a pcDNA3.4 vector (BR196). Hereinafter, antibodies having aT04m02-scFab fused via a linker sequence only at the C-terminus of the second heavy chain constant region are referred to as “aT04m02.1-scFab_mv”. In addition, based on SEQ ID NO: 91, a sequence in which the variable region of the anti-Aβ antibody was replaced with a humanized sequence and in which glutamic acid at residue 123 according to EU numbering was substituted with arginine, and glutamine at residue 124 was substituted with lysine (E123R and Q124K), was synthesized, and a vector expressing the light chain was constructed (DNA sequence: SEQ ID NO: 112; amino acid sequence: SEQ ID NO: 113, in the constant region). Hereinafter, in the antibody names in the tables, antibodies containing K147E and K213E substitutions in the first heavy chain, K147E and K213E substitutions in the second heavy chain, and E123R and Q124K substitutions in the light chain are referred to as “mut”.

Accordingly, vectors expressing the first heavy chain gene, and the second heavy chain and antibody fragment genes of each modified bispecific anti-human TfR/humanized anti-Aβ human IgG1 antibody, were constructed. Note that the first and second heavy chain constant regions of the bispecific anti-human TfR/humanized anti-Aβ human IgG1 antibody consist of the amino acid sequences of SEQ ID NOs: 115 and 117, and the light chain constant region of the same humanized anti-Aβ antibody consists of the amino acid sequence of SEQ ID NO: 119, encoded by the DNA sequences of SEQ ID NOs: 114, 116, and 118 (BR188).

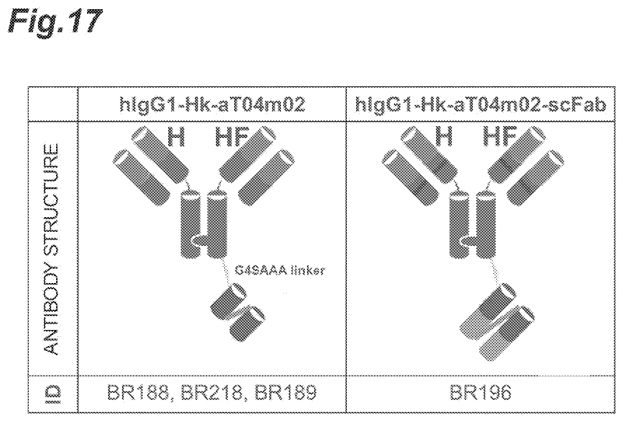

FIG. 17 shows schematic diagrams of the respective antibodies, and Table 14 shows the combinations of the first and second heavy chains and the light chains included in each antibody. Note that, hereinafter, specific sequences and SEQ ID NOs for the variable region of HuAb1 are not shown; however, the variable region of HuAb1 is included in the HuAb1 antibody produced and used in each Experimental Example, as well as in the bispecific antibodies containing HuAb1.

TABLE 14

| ID | Antibody name | Antibody region | DNA/Amino acid | SEQ ID NO: |
|---|---|---|---|---|
| BR218 | HuAb1-hlgG1-N297A-Hk- | First heavy chain (H) | DNA | 104 |
| | aT04m02.1_mv | constant region | Amino | 105 |
| | H; N297A, hole (Y349C, | | acid | |
| | T366S, L368A, Y407V) | Second heavy chain | DNA | 106 |
| | HF; N297A, knob (S354C, | (HF) | Amino | 107 |
| | T366W), linker (G4SAAA), | constant region and | acid | |
| | aT04m02-scFv | scFv region | | |
| | L; wt | Light chain (L) | DNA | 118 |
| | | constant region | Amino | 119 |
| | | | acid | |
| BR189 | HuAb1-hIgG1-cisLALA- | First heavy chain (H) | DNA | 120 |
| | Hk-aT04m02.1 mv | constant region | Amino | 121 |
| | H; hole (Y349C, T366S, | | acid | |
| | L368A, Y407V) | Second heavy chain | DNA | 108 |
| | HF; LALA( L234A, | (HF) | Amino | 109 |
| | L235A), knob (S354C, | constant region and | acid | |
| | T366W), linker (G4SAAA), | scFv region | | |
| | aT04m02-scFv | Light chain (L) | DNA | 118 |
| | L; wt | constant region | Amino | 119 |
| | | | acid | |
| BR196 | HuAb1-hlgG1-mut-Hk- | First heavy chain (H) | DNA | 122 |
| | aT04m02.1 scFab_mv | constant region | Amino | 123 |
| | H; K147E, K213E, hole | | acid | |
| | (Y349C, T366S, L368A, | Second heavy chain | DNA | 110 |
| | Y407V) | (HF) | | |
| | HF; K147E, K213E, knob | constant region and | Amino | 111 |
| | (S354C, T366W), linker | scFab region | acid | |
| | (G4SAAA), aT04m02-scFab | Light chain (L) | DNA | 112 |
| | L; E123R,Q124K | constant region | Amino | 113 |
| | | | acid | |
| BR188 | HuAb1-hlgG1-Hk- | First heavy chain (H) | DNA | 114 |
| | aT04m02.1_mv | constant region | Amino | 115 |
| | H; hole (Y349C, T366S, | | acid | |
| | L368A, Y407V) | Second heavy chain | DNA | 116 |
| | HF; knob (S354C, T366W), | (HF) | | |
| | linker (G4SAAA), | constant region and | Amino | 117 |
| | aT04m02-scFv | scFv region | acid | |
| | L; wt | Light chain (L) | DNA | 118 |
| | | constant region | Amino | 119 |
| | | | acid | |

### (2) Production and Purification of Bispecific Anti-Human TfR/Aβ Human IgG1 Antibodies

The bispecific anti-human TfR/Aβ human IgG1 antibodies BR188, BR218, BR189, and BR196 were transiently produced according to the manufacturer's high-titer protocol using an ExpiCHO expression system (Thermo). The cell culture medium containing the secreted antibodies was collected 11 days after transfection and clarified by removing the cells by centrifugation. The clarified supernatant was purified using a KanCapA3G resin (KANEKA CORPORATION), and the eluate was buffer-exchanged with histidine buffer using dialysis or a desalting column.

### Experimental Example 18: Binding Evaluation to Antigen (Aβ & TfR) (ELISA, SPR, and Flow Cytometry)

### (1) Preparation of TfR Proteins

The His-MBP-human TfR protein including the amino acid sequence corresponding to the extracellular region of human TfR (positions 121 to 760) (SEQ ID NO: 242) was produced and purified according to the procedure described in Experimental Example 1, using an Expi293 expression system (Thermo).

### (2) Preparation of Aβ Protofibril

An Aβ1-42 peptide (Anaspec, Inc.) was dissolved in 10 mM NaOH, and 1/10 volume of 10×PBS was then added to adjust the concentration to 500 μM. Aggregation of Aβ was induced by incubating the solution at 37° C. for several hours. Insoluble precipitates were removed by centrifugation at 16,000×g for 5 minutes, and the resulting soluble aggregates (protofibril) were collected. For the evaluation of protofibril purity, a peak eluting in the void was analyzed using a Superdex75 Increase column (Cytiva, Inc.), and the void peak was fractionated to purify the protofibril as necessary.

### (3) Binding Evaluation to Antigen (Aβ & TfR) (ELISA)

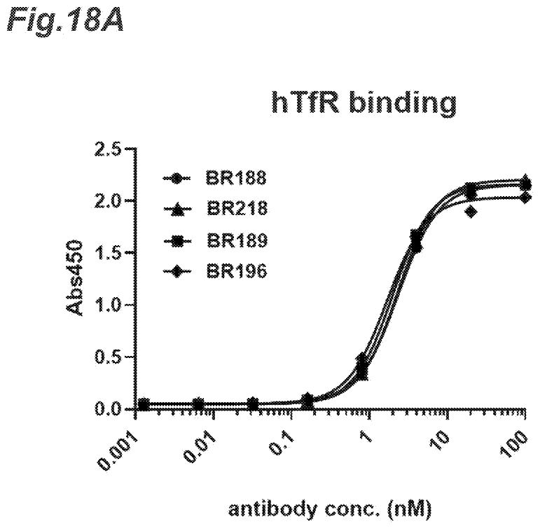

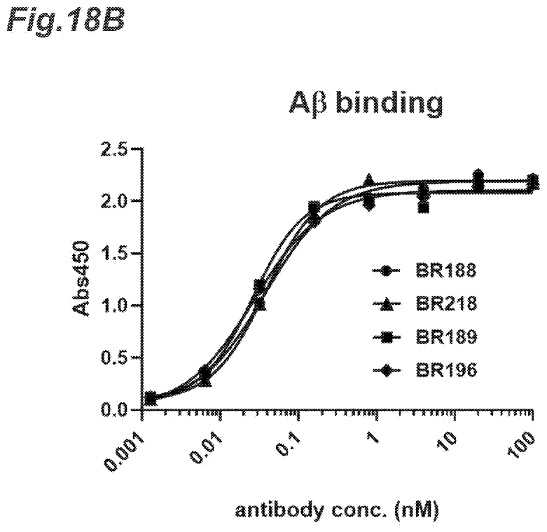

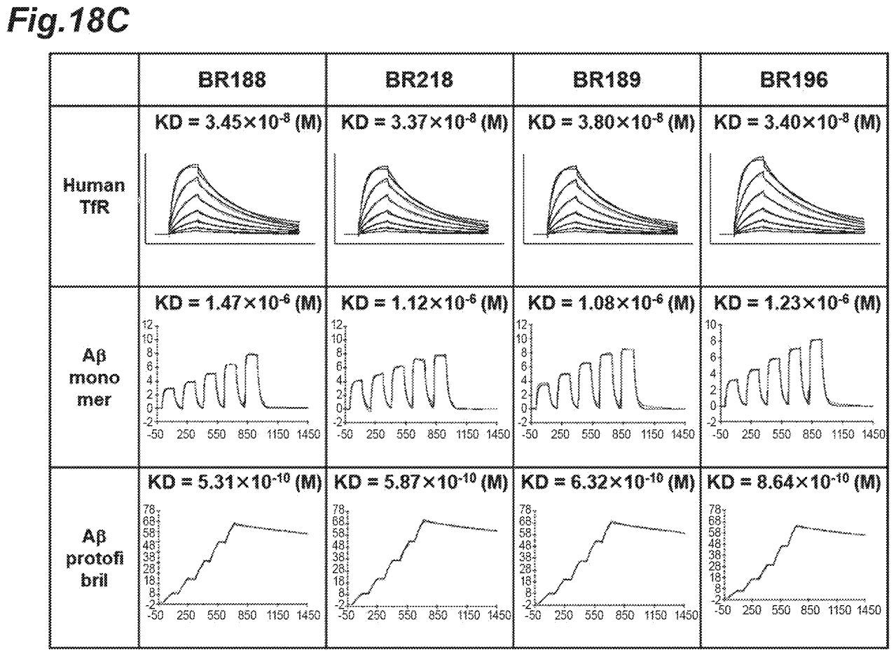

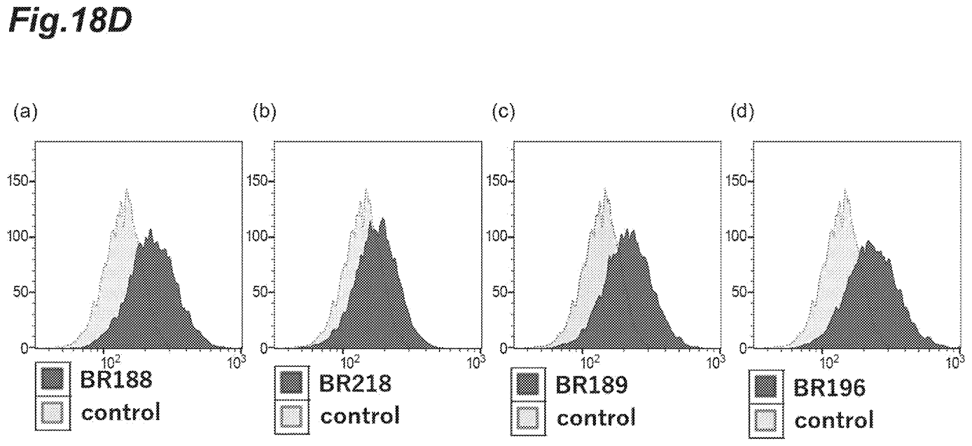

The binding of BR188, BR218, BR189, and BR196 to human TfR was evaluated according to the ELISA steps described in Experimental Example 13, using the His-MBP-human TfR protein (SEQ ID NO: 16) described in Experimental Example 13 as an antigen. The graph of the absorbance of various antibody concentrations at 450 nm is shown in FIG. 18A. FIG. 18A shows that all the bispecific anti-human TfR/humanized Aβ antibodies have equivalent binding properties to human TfR.

The binding of BR188, BR218, BR189, and BR196 to protofibril-like Aβ was evaluated according to the ELISA steps described in Experimental Example 13. The graph of the absorbance of various antibody concentrations at 450 nm is shown in FIG. 18B. FIG. 18B shows that all the bispecific anti-human TfR/humanized Aβ antibodies have equivalent binding properties to protofibril-like Aβ.

### (4) SPR-Aβ

The measurement of the binding affinity of BR188, BR218, BR189, and BR196 to monomer Aβ was performed by surface plasmon resonance (SPR method, Biacore S200, Cytiva, Inc.) below. Immobilization of BR188, BR218, BR189, and BR196 on a Series S CM5 sensor chip was performed by a standard amine coupling method according to the vendor's manual. Next, a dilution series of monomer Aβ were sequentially injected to respective antibodies, and the binding reactions were observed. Analysis of sensorgram by Biacore S200 Evaluation Software (Cytiva, Inc.) was performed to calculate the binding affinity (KD value) by the 1:1 binding model. The calculated KD values and binding response curves are shown in FIG. 18C. As shown in FIG. 18C, all the bispecific anti-human TfR/humanized Aβ antibodies exhibited equivalent binding properties to monomer Aβ.

The measurement of the binding affinity of BR188, BR218, BR189, and BR196 to Aβ protofibril was also performed by SPR method. Immobilization of Aβ protofibril on a Series S CM5 sensor chip was performed by a standard amine coupling method according to the vendor's manual. Next, a dilution series of BR188, BR218, BR189, and BR196 was sequentially injected, and the binding reactions were observed. Analysis of sensorgram by Biacore S200 Evaluation Software was performed to calculate the binding affinity (KD value) by the bivalent analyte model. The calculated KD values and binding response curves are shown in FIG. 18C. As shown in FIG. 18C, all the bispecific anti-human TfR/humanized Aβ antibodies exhibited equivalent binding affinities to Aβ protofibril.

### (5) SPR-TfR

The measurement of the binding affinity of BR188, BR218, BR189, and BR196 to human TfR was performed by surface plasmon resonance (SPR method, Biacore T200, Cytiva, Inc.) below. The His-MBP-hTfR protein (SEQ ID NO: 242) that was diluted with HBS-EP+buffer solution was injected and captured on the anti-His antibody which was immobilized on a Series S CM5 sensor chip. Immobilization of the anti-His antibody was performed by a standard amine coupling method according to the vendor's manual. Next, a dilution series of BR188, BR218, BR189, and BR196, prepared in HBS-EP+buffer was sequentially injected, and the binding reactions for 120 seconds for the binding phase and 420 seconds for the dissociation phase were observed. Analysis of sensorgram by BIA evaluation Software ver. 2.0 (Cytiva, Inc.) was performed to calculate the binding affinity (KD value) by the 1:1 binding model. The calculated KD values and binding response curves are shown in FIG. 18C below. As shown in FIG. 18C, all the bispecific anti-human TfR/humanized Aβ antibodies exhibited equivalent binding properties to human TfR.

### (6) Flow Cytometry Analysis

Flow cytometry analysis using U2OS cells was performed according to the following steps. U2OS cells were washed twice with wash buffer (1% FBS (NICHIREI CORPORATION)/1 mM EDTA/PBS/0.02% sodium azide (FUJIFILM Wako Pure)), resuspended in FcR Blocking Reagent, human (Miltenyi)/wash buffer and incubated on ice for 10 minutes. After that, BR188, BR218, BR189, and BR196 prepared in wash buffer, were added and incubated on ice for 30 minutes. The Cells were washed three times and then incubated on ice for 30 minutes with fluorescently labeled secondary antibody (Jackson ImmunoResearch Inc.) prepared in 5% rat mouse serum/wash buffer. After washing three times, fluorescence signals were detected using LSRFortessa X-20 (BD and Company). Flow cytometry results are shown in FIG. 18D. FIG. 18D confirms that the bispecific anti-human TfR/humanized Aβ antibodies BR188, BR218, BR189, and BR196 exhibited comparable binding to U2OS cells.

### Experimental Example 19: Evaluation of Fc-Mediated Effector Function (SPR-FcRs, ADCC, C1q Binding, and Ex Vivo Amyloid Plaque Phagocytosis)

### (1) SPR-FcRs

The binding affinity of BR188, BR218, BR189, and BR196 to human FcγRI, human FcγRIIA, and human FcγRIIIA was measured by surface plasmon resonance (SPR method, Biacore T200, Cytiva, Inc.) below. The human FcγRI-His, human FcγRIIA-His, or human FcγRIIIA-His (Sino Biological, Inc. for all) that was diluted with HBS-EP+buffer solution was injected and captured on the anti-His antibody which was immobilized on a Series S CM5 sensor chip. Immobilization of the anti-His antibody was performed by a standard amine coupling method according to the vendor's manual. Next, a dilution series of BR188, BR218, BR189, and BR196 was sequentially injected, and the binding reactions were observed. Analysis of sensorgram by BIAevaluation Software ver. 2.0 (Cytiva, Inc.) was performed to calculate the binding affinity (KD value) of each antibody by the 1:1 binding model.

The binding affinity of BR188, BR218, BR189, and BR196 to human FcRn was measured by surface plasmon resonance below. A solution containing the human FcRn-His (Sino Biological, Inc.) was injected, and the human FcRn-His was immobilized on a Series S CM5 sensor chip. Immobilization was performed by a standard amine coupling method according to the vendor's manual. Next, a dilution series of BR188, BR218, BR189, and BR196 was sequentially injected, and the binding reactions were observed. Analysis of sensorgram by BIA evaluation Software ver. 2.0 (Cytiva, Inc.) was performed to calculate the KD value of each antibody by the 1:1 binding model.

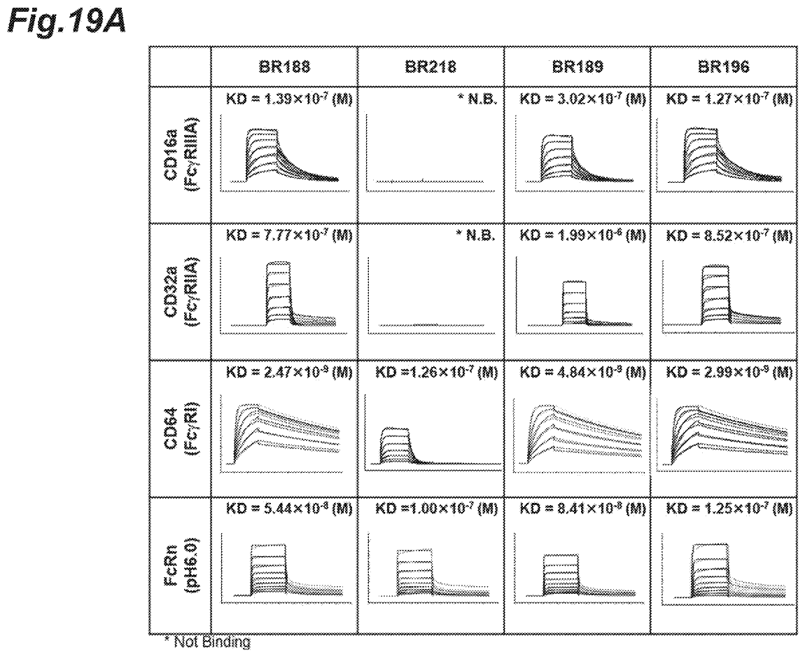

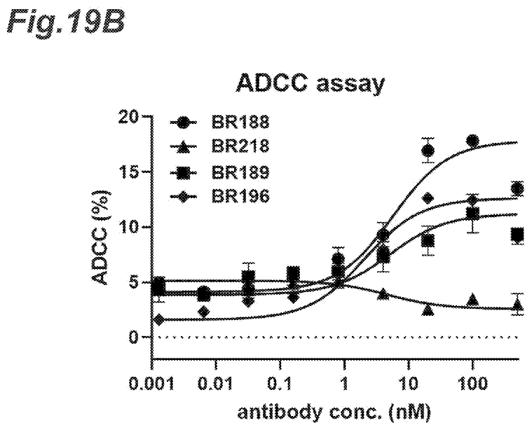

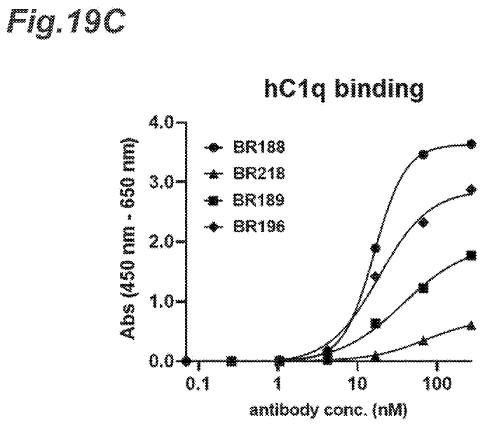

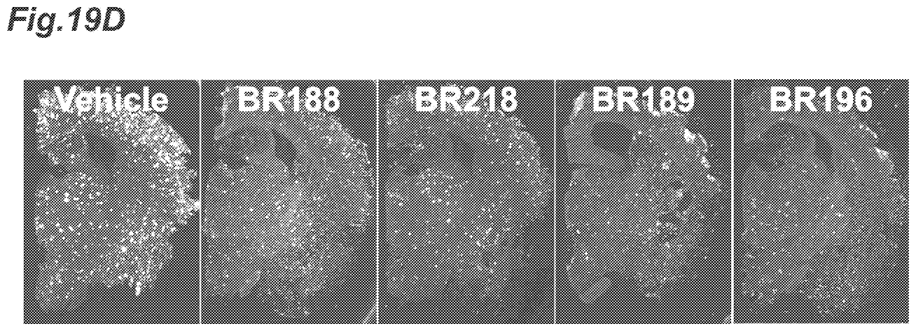

The binding curves and KD values are shown in FIG. 19A. As shown in FIG. 19A, compared with BR188, BR218 having the N297A substitution did not show detectable binding properties to human FcγRIIA and human FcγRIIIA, while exhibiting reduced binding to human FcγRI. In contrast, all antibodies showed similar KD values for the binding to human FcRn.

### (2) Evaluation of Antibody-Dependent Cellular Cytotoxicity (ADCC) Activity

U2OS cells were seeded at 6.25×10³ cells/well in 96-well culture plates, using culture medium (10% FBS (NICHIREI CORPORATION)/Kanamycin Sulfate (GIBCO)/McCoy's 5A (GIBCO)), and cultured overnight at 37° C. 5% CO₂. Human PBMC, ADCC-Qualified (Promega Corporation) were thawed and cultured overnight at 37° C. 5% CO₂ in culture medium (10% FBS/1 mM sodium pyruvate (Sigma Aldrich Co. LLC) 5 ng/ml IL-2 (PeproTech, Inc.)/55 μM 2-mercaptoethanol/Antibiotic-Antimycotic (GIBCO)/RPMI-1640 (GIBCO)). On Day 1 of culture, wells containing U2OS cells were washed twice with assay buffer (1% FBS/5 ng/ml IL-2/Antibiotic-Antimycotic/RPMI-1640), and BR188, BR218, BR189, and BR196 prepared in assay buffer were added to the plate containing U2OS cells and incubated at 37° C. 5% CO₂ for 30 minutes. Then, PBMC were added at 1.5625×10⁵ cells/well using assay buffer and incubated at 37° C. 5% CO₂ for four hours. After incubation, centrifugation was performed to collect supernatant. An equal volume of Cytotoxicity Detection Kit (Roche) was added to the supernatant, incubated in the dark at room temperature for 30 minutes, and the reaction was stopped with 1N hydrochloric acid, and lactate dehydrogenase (LDH) activity was then measured. Absorbance at 492 nm (with background at 620 nm subtracted) was measured using an EnVision 2105 microplate reader (PerkinElmer, Inc.). LDH absorbance released spontaneously from U2OS cells was defined as (TSR), LDH absorbance released from all U2OS cells lysed with Triton (trademark) X-100 as (TMR), LDH absorbance present in assay buffer as (CMB), LDH absorbance released spontaneously from PBMC as (ESR), and LDH absorbance when Triton (trademark) X-100 was added to assay buffer as (VCC), % ADCC was calculated and shown in FIG. 19B. FIG. 19B indicates that the ADCC activity of BR218 having N297A was markedly lower than that of BR188.

$$\% ADCC ( cytotoxicity ) = [ ( Sample - CMB ) - ( ESR - CMB ) - ( TSR - CMB ) ] / [ ( TMR - VCC ) - ( TSR - CMB ) ] \times 100$$

### (3) Binding Evaluation to C1q

The binding of BR188, BR218, BR189, and BR196 to human C1q was evaluated by ELISA below. Antibody solutions prepared as dilution series were added to Immuno plate MaxiSorp (Thermo) and incubated overnight at 4° C. to immobilize the antibodies. After removing the antibody solutions, 0.5% BSA/0.4% I-Block (Thermo)/0.05% Tween20/PBS was added, and blocking was performed at room temperature for one hour. After washing the plate with 0.05% Tween20/PBS three times, human C1q (ProSpec-Tany TechnoGene Ltd.) was added to each well and incubated at room temperature for 1.5 hours. After washing with 0.05% Tween20/PBS three times, human C1q bound to each antibody was detected by HRP Anti-C1q antibody (Abcam plc). TMBZ (SeraCare) was used as the chromogenic substrate, and the reaction was terminated by addition of sulfuric acid. Absorbance values at 450 nm, corrected by subtraction of background at 650 nm, were plotted as shown in FIG. 19C. As shown in FIG. 19C, BR218 having the N297A substitution exhibited markedly reduced binding to human C1q compared with BR188.”

### (4) Ex Vivo Amyloid Plaque Phagocytosis

Mouse microglia were prepared by the following steps as phagocytes. Two litters of newborn C57BL/6 mice obtained from parent mice were decapitated, and then whole brains were harvested and transferred to Leibovitz's L-15 medium (Gibco). The cerebrum region was isolated from the whole brain, transferred to fresh Leibovitz's L-15 medium, and minced with scissors. The minced tissue fragments were transferred to a 50 mL tube, allowed to settle naturally, and then the supernatant was removed. To the tissue fragments, 14 mL of 0.25% Trypsin (Gibco) and 300 μL of DNase I (Sigma Aldrich Co. LLC) solution prepared at 15,000 units/mL were added. After incubating at 37° C. for 30 minutes, centrifugation was performed at 260× g for 3 minutes at room temperature. The supernatant was removed, and the pellet was resuspended in 10 mL of DMEM/F12 medium (Gibco) containing 10% fetal bovine serum and penicillin-streptomycin (FUJIFILM Wako Pure), hereafter referred to as medium, supplemented with 100 μL of DNase I solution. After centrifuging again at 260× g for 3 minutes at room temperature, the supernatant was removed, and the pellet was resuspended in 10 mL of medium supplemented with 100 μL of DNase I solution. The number of viable cells was calculated, and the cells were seeded into 3 flasks at a density of at least 880×10⁴ cells/225 cm², and cultured at 37.0° C. in a 5.0% CO₂ incubator. Three days after starting the culture, the medium was replaced with fresh medium containing 5 ng/ml of gm-CSF (R&D Systems, Inc.). Afterward, the medium was replaced twice a week to promote microglial proliferation. After the proliferation of microglia, cells were collected during medium exchange performed twice a week, centrifuged at 260×g for 3 minutes at room temperature, and resuspended in medium containing 5 ng/mL gm-CSF for use in the assay. As targets of phagocytosis, 10-month-old female APP KI mice were used. Exsanguination by right auricle incision and transcardial perfusion with heparin-containing PBS were performed, and the brain was collected. The brain was cut in half, fixed at 4° C. for 24 hours in 4% paraformaldehyde phosphate buffer solution, then washed with PBS (−), and frozen in liquid nitrogen. Brain sections at a thickness of 20 μm were prepared using a CM1860 cryostat (Leica), mounted on poly-D-lysine-coated 12 mm coverslips (neu Vitro Corporation), placed in a 24-well plate, air dried at 45° C., and washed with PBS (−). Sections were incubated at room temperature for one hour with 300 μg/mL of BR188, BR218, BR189, or BR196 and then washed twice with PBS (−). Mouse microglia were added at 5×10⁵ cells/well, and cultured at 37° C. in 5% CO₂ for 3 days. After culture, the samples were washed twice with PBS (−) and fixed on ice for 10 minutes with 1% paraformaldehyde phosphate buffer solution. After washing twice with PBS (−), blocking was performed at room temperature for 30 minutes with 5% Block Ace (DS Pharma Biomedical Co., Ltd.) and 0.1% Tween20 in PBS (−). Anti-Human Amyloid beta (N) (82E1) Mouse IgG MoAb (IBL Co., Ltd.), fluorescently labeled using Alexa Fluor 488 Antibody Labeling Kit (Molecular probes) was diluted with 1% Block Ace and 0.1% Tween20 in PBS (−), mounted on coverslips, and incubated at room temperature for one hour. After washing three times with PBS (−), the samples were mounted using Prolong Gold Antifade Mountant (Molecular probes), and imaged with a 4× objective lens of an all-in-one fluorescence microscope BZ-X710 (KEYENCE Corporation) (FIG. 19D). Compared with vehicle-treated sections, sections treated with BR188, BR218, BR189, and BR 196 showed a clear reduction in anti-amyloid beta antibody-positive signals.

### Experimental Example 20: Mouse PK

(1) Sample Preparation from Bispecific Anti-Human TfR/Aβ Human IgG1 Antibody Treated Animals

To 2-month-old male hTfR-KI (homo) mice, the bispecific anti-human TfR/Aβ human IgG1 antibodies BR188, BR218, BR189, and BR196 were administered via the tail vein at a dose of 20 mg/kg (10 mL/kg), calculated as an IgG equivalent amount and after 1, 3, and 7 days, blood was collected under anesthesia, followed by exsanguination via incision of the right atrial appendage and transcardial perfusion with heparin-containing PBS. The brain was harvested and divided into the left and right cerebral hemispheres. For the right cerebral hemisphere, the weight of the wet tissues was measured, frozen in liquid nitrogen, and stored at −80° C. The collected blood was centrifuged at 1,200×g for 15 minutes at 4° C., and the supernatant was collected as plasma.

### (2) Measurement of Antibody Concentration

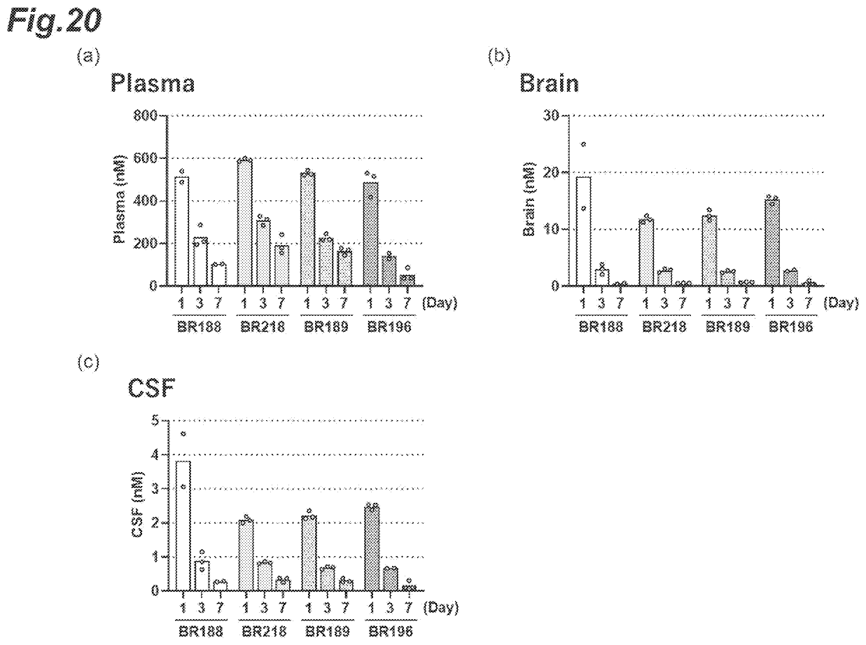

The frozen right cerebral hemisphere collected in (1) was thawed and homogenized by a BioMasher I (Nippi Inc.). It was suspended with 1% NP-40 (Sigma Aldrich Co. LLC)/TBS/complete (Roche) and incubated at 4° C. for one hour, and then centrifuged at 15,000 rpm for 10 minutes at 4° C. to collect the supernatant as a brain homogenate. The measurement of the antibody concentrations in plasma, cerebrospinal fluid (CSF), and brain homogenate was performed by ELISA as described below. A donkey anti-human IgG-Fcγ chain-specific F(ab′)2 antibody (Jackson ImmunoResearch Inc.) was added to a MaxiSorp Plates (Thermo) and incubated overnight at 4° C. After washing with 0.02% Tween20/PBS three times, blocking was performed at room temperature for one hour by 1% Block Ace (DS Pharma Biomedical Co., Ltd.), and then, plasma, CSF, or the brain homogenate diluted using 0.4% Block Ace were added and incubated at room temperature for 2 hours. After washing with 0.02% Tween20/PBS three times, an HRP-labeled goat anti-human IgG Fcγ chain-specific F(ab′)2 antibody (Jackson ImmunoResearch Inc.) was added and incubated at room temperature for one hour. After washing with 0.02% Tween20/PBS three times, TMB substrate solution (SeraCare) was added and incubated at room temperature for 10 minutes. An equivalent amount of 2N sulfuric acid was added to quench the reaction, and the absorbance at 450 nm and 650 nm was measured with a microplate reader SpectraMax ABS (Molecular Devices, LLC). Calibration curves were created based on the absorbance of standard samples at each concentration, and the antibody concentrations of the respective samples were determined (FIG. 20). From the antibody concentrations in the brain, plasma, and CSF at 1, 3, and 7 days post-administration of BR188, BR218, BR189, and BR196, it was confirmed that the brain-penetrating properties of all the bispecific antibodies are high. In addition, no marked differences in the brain-penetrating properties between these four antibodies were observed.

### Experimental Example 21: Production of Modified Bispecific Anti-Human TfR/Aβ Human IgG1 Antibody

### Construction of Modified Bispecific Anti-Human TfR/Aβ Human IgG1 Antibody Gene Expression Vector

Similarly to Experimental Example 1, electrostatic steering was applied as a dimerization technique to the DNA sequences encoding the first and second heavy chains of the humanized anti-Aβ human IgG1 antibody. Specifically, two amino-acid substitutions were introduced into the CH3 domain of one of the heavy chains—lysine at residue 409 according to EU numbering was substituted with aspartic acid (K409D), and lysine at residue 439 was substituted with glutamic acid (K439E)—and the corresponding two amino-acid substitutions were introduced into the CH3 domain of the other heavy chain-aspartic acid at residue 356 was substituted with lysine (D356K), and aspartic acid at residue 399 was substituted with lysine (D399K). Further, into the DNA sequence encoding the second heavy chain, a DNA sequence encoding aT04m02-scFv or aT04m0206-scFv was inserted at the C-terminus via a G4S linker. Accordingly, vectors expressing the first heavy chain gene (DNA sequence: SEQ ID NO: 124; amino acid sequence: SEQ ID NO: 125, in the constant region) and the second heavy chain and scFv gene (DNA sequence: SEQ ID NO: 126; amino acid sequence: SEQ ID NO: 127, in the constant region and scFv region) of the bispecific anti-human TfR/humanized Aβ human IgG1 antibody containing aT04m02-scFv at the C-terminus and including K409D and K439E mutations in the first heavy chain and D356K and D399K mutations in the second heavy chain were constructed (BR258). Hereinafter, the mutations K409D and K439E in the first heavy chain and D356K and D399K in the second heavy chain are referred to as “Hp” or “Hp mutation.” Similarly, vectors expressing the first heavy chain gene (DNA sequence: SEQ ID NO: 128; amino acid sequence: SEQ ID NO: 129, in the constant region) and the second heavy chain and scFv gene (DNA sequence: SEQ ID NO: 130; amino acid sequence: SEQ ID NO: 131, in the constant region and scFv region) of the bispecific anti-human TfR/humanized Aβ human IgG1 antibody containing aT04m02-scFv at the C-terminus and including D356K and D399K mutations in the first heavy chain and K409D and K439E mutations in the second heavy chain were constructed (BR259). Hereinafter, the mutations D356K and D399K in the first heavy chain and K409D and K439E in the second heavy chain are referred to as “Hn” or “Hn mutation.” In addition, a vector expressing the second heavy chain and scFv gene (DNA sequence: SEQ ID NO: 132; amino acid sequence: SEQ ID NO: 133, in the constant region and scFv region) of the bispecific anti-human TfR/humanized Aβ human IgG1 antibody containing aT04m0206-scFv at the C-terminus and the Hn mutation was constructed (BR350). Furthermore, in the vectors expressing the second heavy chain and scFv genes containing the amino acid sequences of SEQ ID NOs: 131 and 133, DNA sequences encoding mutants in which glycine at the ninth residue from the N-terminus of HFR2 (heavy chain framework region 2) and glycine at the third residue from the N-terminus of LFR4 (light chain framework region 4), defined by Kabat numbering, were substituted with cysteine (DNA sequences: SEQ ID NOs: 134 and 136; amino acid sequences: SEQ ID NOs: 135 and 137, in the constant region and dsscFv region) were synthesized de novo and cloned into a pcDNA3.4 vector. Accordingly, expression vectors for bispecific anti-human TfR/humanized Aβ human IgG1 antibodies containing dsscFv were constructed (BR357 and BR368). Hereinafter, in the antibody names in the tables, antibodies in which an scFv containing a disulfide bond formed by the two cysteine substitutions is fused only to the second heavy chain are referred to as “dsscFv.”

In the vectors expressing the second heavy chain and scFv containing the amino acid sequence of SEQ ID NO: 131, or the second heavy chain and dsscFv containing the amino acid sequence of SEQ ID NO: 135, DNA sequences encoding new second heavy chains and scFv, or new second heavy chains and dsscFv, in which the order of the heavy chain variable region and the light chain variable region of aT04m02-scFv or aT04m02-dsscFv was switched, were synthesized (DNA sequences: SEQ ID NOs: 138 and 140; amino acid sequences: SEQ ID NOs: 139 and 141, in the constant region and scFv or dsscFv region) (BR355 and BR356). Hereinafter, in the antibody names in the tables, the order of the light chain and heavy chain variable regions is described as “LH.” In addition, in the vectors expressing the second heavy chain and dsscFv containing the amino acid sequence of SEQ ID NO: 135, and the second heavy chain and dsscFv containing the amino acid sequence of SEQ ID NO: 141, DNA sequences encoding new second heavy chains and Fv heavy chain or Fv light chain genes, each having only the heavy chain or light chain variable region of aT04m02-dsscFv in which the amino acid sequences following the glycine- and serine-based linker connecting the heavy chain and light chain variable regions of the modified aT04m02-dsscFv were removed, were synthesized (DNA sequences: SEQ ID NOs: 142 and 144; amino acid sequences: SEQ ID NOs: 143 and 145, in the constant region and Fv heavy chain or Fv light chain region), together with the light chain or heavy chain variable region of aT04m02-dsscFv (DNA sequences: SEQ ID NOs: 146 and 245; amino acid sequences: SEQ ID NOs: 147 and 246) (BR359 and BR358). Hereinafter, in the antibody names in the tables, constructs in which the glycine- and serine-based linker connecting the heavy chain and light chain variable regions of dsscFv is removed are described as “dsFv.” Furthermore, in the vector expressing the second heavy chain and scFv containing the amino acid sequence of SEQ ID NO: 131, DNA sequences encoding new second heavy chains and scFv in which leucine at the fifth position from the N-terminus of HFR1 (heavy chain framework region 1) of the aT04m02-scFv sequence, defined by Kabat numbering, was substituted with valine (L5V), glutamine (L5Q), glutamic acid (L5E), or lysine (L5K), were synthesized (DNA sequences: SEQ ID NOs: 148, 150, 152, and 154; amino acid sequences: SEQ ID NOs: 149, 151, 153, and 155, in the constant region and scFv region) (BR360, BR361, BR362, and BR363). Hereinafter, aT04m02-scFv including these mutations is described as “aT04m02.1-L5X” (X is any of V, Q, E, or K). In addition, DNA sequences encoding new second heavy chains and scFv having a linker sequence in which three consecutive alanine residues in the linker connecting aT04m02-scFv to the heavy chain C-terminus were substituted with two consecutive glycine residues followed by serine were synthesized (DNA sequence: SEQ ID NO: 156; amino acid sequence: SEQ ID NO: 157, in the constant region and scFv region) (BR364). Hereinafter, in the antibody names in the tables, antibodies having aT04m02-scFv fused via a linker sequence substituted with two consecutive glycine residues followed by serine at the C-terminus of the second heavy chain constant region are described as “GGS-aT04m02.1.” All of these DNA sequences were synthesized de novo and cloned in the same manner, thereby constructing expression vectors for the bispecific anti-human TfR/humanized Aβ human IgG1 antibody genes.

In the vector expressing the second heavy chain and scFv gene containing the amino acid sequence of SEQ ID NO: 131, a DNA sequence in which a single amino-acid substitution—lysine at residue 370 according to EU numbering substituted with glutamic acid (K370E)—was additionally introduced into the CH3 domain of the second heavy chain was synthesized (DNA sequence: SEQ ID NO: 160; amino acid sequence: SEQ ID NO: 161, in the constant region and scFv region). In the vector expressing the first heavy chain gene containing the amino acid sequence of SEQ ID NO: 129, a DNA sequence in which a single amino-acid substitution—glutamic acid at residue 357 according to EU numbering substituted with lysine (E357K)—was additionally introduced into the CH3 domain, and in which the C-terminal lysine was removed (hereinafter referred to as “HΔK”), was fully synthesized (DNA sequence: SEQ ID NO: 158; amino acid sequence: SEQ ID NO: 159, in the constant region) (BR380). Hereinafter, mutations D356K, E357K, and D399K in the first heavy chain, together with K370E, K409D, and K439E in the second heavy chain, are referred to as “Hn3” or “Hn3 mutation.” Similarly, mutations K370E, K409D, and K439E in the first heavy chain, together with D356K, E357K, and D399K in the second heavy chain, are referred to as “Hp3” or “Hp3 mutation.” The description “HΔK” in the antibody names in the tables indicates that the first heavy chain contains the HΔK modification. Further, in the second heavy chain containing the amino acid sequence of SEQ ID NO: 161, DNA sequences encoding mutants in which glycine at the seventh position of HFR2 (heavy chain framework region 2) and glycine at the third position of LFR4 (light chain framework region 4) of aT04m02-dsscFv, defined by Kabat numbering, were substituted with cysteine, were synthesized and cloned into a pcDNA3.4 vector. Accordingly, an expression vector for the bispecific anti-human TfR/humanized Aβ human IgG1 antibody gene was constructed (DNA sequence: SEQ ID NO: 162; amino acid sequence: SEQ ID NO: 163, in the constant region and dsscFv region) (BR397). Thus, vectors expressing the first heavy chain gene, the second heavy chain and antibody-fragment genes, and the light chain gene of each modified bispecific anti-human TfR/humanized Aβ human IgG1 antibody were constructed.

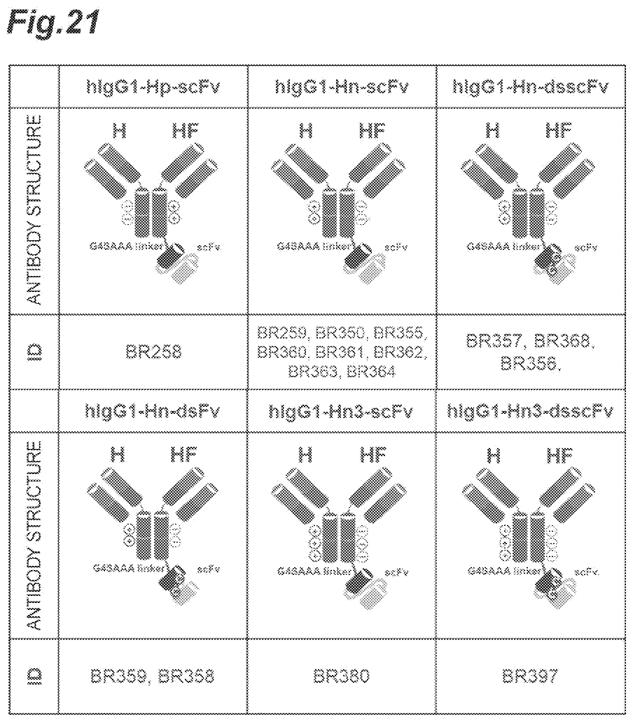

Schematic diagrams of the respective antibodies and the combinations of the first and second heavy chains and the light chains included in each antibody are shown in FIG. 21 and Table 15, respectively.

TABLE 15

| | | | DNA/ | SEQ |
|---|---|---|---|---|
| | | | Amino | ID |
| ID | Antibody name | Antibody region | acid | NO: |
| BR258 | HuAb1-hlgG1-N297A-Hp- | First heavy | DNA | 124 |
| | aT04m02.1 mv | chain (H) | Amino | 125 |
| | H; N297A, K409D, K439E | constant region | acid | |
| | HF; N297A, D356K, | Second heavy | DNA | 126 |
| | D399K, linker (G4SAAA), | chain (HF) | Amino | 127 |
| | aT04m02-scFv | constant region | acid | |
| | L; wt | and | | |
| | | scFv region | | |
| | | Light chain (L) | DNA | 118 |
| | | constant region | Amino | 119 |
| | | | acid | |
| BR259 | HuAb1-hlgG1-N297A-Hn- | First heavy | DNA | 128 |
| | aT04m02.1 mv | chain (H) | Amino | 129 |
| | H; N297A, D356K, D399K | constant region | acid | |
| | HF; N297A, K409D, K439E, | Second heavy | DNA | 130 |
| | linker (G4SAAA), aT04m02- | chain (HF) | Amino | 131 |
| | scFv | constant region | acid | |
| | L; wt | and | | |
| | | scFv region | | |
| | | Light chain (L) | DNA | 118 |
| | | constant region | Amino | 119 |
| | | | acid | |
| BR350 | HuAb1-hIgG1-N297A-Hn- | First heavy | DNA | 128 |
| | aT04m0206.1_mv | chain (H) | Amino | 129 |
| | H; N297A, D356K, D399K | constant region | acid | |
| | HF; N297A, K409D, K439E, | Second heavy | DNA | 132 |
| | linker (G4SAAA), | chain (HF) | Amino | 133 |
| | aT04m0206-scFv | constant region | acid | |
| | L; wt | and | | |
| | | scFv region | | |
| | | Light chain (L) | DNA | 118 |
| | | constant region | Amino | 119 |
| | | | acid | |
| BR355 | HuAb1-hIgG1-N297A-Hn- | First heavy | DNA | 128 |
| | aT04m02.1-LH_mv | chain (H) | Amino | 129 |
| | H; N297A, D356K, D399K | constant region | acid | |
| | HF; N297A, K409D, K439E, | Second heavy | DNA | 138 |
| | linker (G4SAAA), aT04m02- | chain (HF) | Amino | 139 |
| | LH-scFv | constant region | acid | |
| | L; wt | and | | |
| | | scFv region | | |
| | | Light chain (L) | DNA | 118 |
| | | constant region | Amino | 119 |
| | | | acid | |
| BR357 | HuAb1-hlgG1-N297A-Hn- | First heavy | DNA | 128 |
| | aT04m02.1_dsscFv | chain (H) | Amino | 129 |
| | H; N297A, D356K, D399K | constant region | acid | |
| | HF; N297A, K409D, | Second heavy | DNA | 134 |
| | K439E, linker (G4SAAA), | chain (HF) | Amino | 135 |
| | aT04m02-dsscFv | constant region | acid | |
| | L; wt | and | | |
| | | dsscFv region | | |
| | | Light chain (L) | DNA | 118 |
| | | constant region | Amino | 119 |
| | | | acid | |
| BR356 | HuAb1-hIgG1-N297A-Hn- | First heavy | DNA | 128 |
| | aT04m02.1-LH_dsscFv | chain (H) | Amino | 129 |
| | H; N297A, D356K, D399K | constant region | acid | |
| | HF; N297A, K409D, | Second heavy | DNA | 140 |
| | K439E, linker (G4SAAA), | chain (HF) | Amino | 141 |
| | aT04m02-LH-dsscFv | constant region | acid | |
| | L; wt | and | | |
| | | dsscFv region | | |
| | | Light chain (L) | DNA | 118 |
| | | constant region | Amino | 119 |
| | | | acid | |
| BR359 | HuAb1-hlgG1-N297A-Hn- | First heavy | DNA | 128 |
| | aT04m02 dsFv | chain (H) | Amino | 129 |
| | H; N297A, D356K, D399K | constant region | acid | |
| | HF; N297A, K409D, | Second heavy | DNA | 142 |
| | K439E, linker (G4SAAA), | chain (HF) | Amino | 143 |
| | aT04m02-VH-cys | constant region | acid | |
| | L; wt | and | | |
| | FvL; aT04m02-VL-cys | Fv heavy chain | | |
| | | (FvH) region | | |
| | | Light chain (L) | DNA | 118 |
| | | constant region | Amino | 119 |
| | | | acid | |
| | | FV light chain | DNA | 146 |
| | | (FvL) region | Amino | 147 |
| | | | acid | |
| BR358 | HuAb1-hIgG1-N297A-Hn- | First heavy | DNA | 128 |
| | aT04m02-LH dsFv | chain (H) | Amino | 129 |
| | H; N297A, D356K, D399K | constant region | acid | |
| | HF; N297A, K409D, | Second heavy | DNA | 144 |
| | K439E, linker (G4SAAA), | chain (HF) | Amino | 145 |
| | aT04m02-VL-cys | constant region | acid | |
| | L; wt | and | | |
| | FvL; aT04m02-VH-cys | FV light chain | | |
| | | (FvL) region | | |
| | | Light chain (L) | DNA | 118 |
| | | constant region | Amino | 119 |
| | | | acid | |
| | | Fv heavy chain | DNA | 245 |
| | | (FvH) region | Amino | 246 |
| | | | acid | |
| BR360 | HuAb1-hIgG1-N297A-Hn- | First heavy | DNA | 128 |
| | aT04m02.1_L5V_mv | chain (H) | Amino | 129 |
| | H; N297A, D356K, D399K | constant region | acid | |
| | HF; N297A, K409D, | Second heavy | DNA | 148 |
| | K439E, linker (G4SAAA), | chain (HF) | Amino | 149 |
| | aT04m02 L5V-scFv | constant region | acid | |
| | L; wt | and | | |
| | | scFv region | | |
| | | Light chain (L) | DNA | 118 |
| | | constant region | Amino | 119 |
| | | | acid | |
| BR361 | HuAb1-hIgG1-N297A-Hn- | First heavy | DNA | 128 |
| | aT04m02.1_L5Q_mv | chain (H) | Amino | 129 |
| | H; N297A, D356K, D399K | constant region | acid | |
| | HF; N297A, K409D, | Second heavy | DNA | 150 |
| | K439E, linker (G4SAAA), | chain (HF) | Amino | 151 |
| | aT04m02_L5Q-scFv | constant region | acid | |
| | L; wt | and | | |
| | | scFv region | | |
| | | Light chain (L) | DNA | 118 |
| | | constant region | Amino | 119 |
| | | | acid | |
| BR362 | HuAb1-hIgG1-N297A-Hn- | First heavy | DNA | 128 |
| | aT04m02.1_L5E_mv | chain (H) | Amino | 129 |
| | H; N297A, D356K, D399K | constant region | acid | |
| | HF; N297A, K409D, | Second heavy | DNA | 152 |
| | K439E, linker (G4SAAA), | chain (HF) | Amino | 153 |
| | aT04m02_L5E-scFv | constant region | acid | |
| | L; wt | and | | |
| | | scFv region | | |
| | | Light chain (L) | DNA | 118 |
| | | constant region | Amino | 119 |
| | | | acid | |
| BR363 | HuAb1-hlgG1-N297A-Hn- | First heavy | DNA | 128 |
| | aT04m02.1_L5K_mv | chain (H) | Amino | 129 |
| | H; N297A, D356K, D399K | constant region | acid | |
| | HF; N297A, K409D, | Second heavy | DNA | 154 |
| | K439E, linker (G4SAAA), | chain (HF) | Amino | 155 |
| | aT04m02 L5K-scFv | constant region | acid | |
| | L; wt | and | | |
| | | scFv region | | |
| | | Light chain (L) | DNA | 118 |
| | | constant region | Amino | 119 |
| | | | acid | |
| BR364 | HuAb1-hIgG1-N297A-Hn- | First heavy | DNA | 128 |
| | GGS-aT04m02.1_mv | chain (H) | Amino | 129 |
| | H; N297A, D356K, D399K | constant region | acid | |
| | HF; N297A, K409D, K439E, | Second heavy | DNA | 156 |
| | linker (GGS), aT04m02-scFv | chain (HF) | Amino | 157 |
| | L; wt | constant region | acid | |
| | | and | | |
| | | scFv region | | |
| | | Light chain (L) | DNA | 118 |
| | | constant region | Amino | 119 |
| | | | acid | |
| BR368 | HuAb1-hIgG1-N297A-Hn- | First heavy | DNA | 128 |
| | aT04m0206.1_dsscFv | chain (H) | Amino | 129 |
| | H; N297A, D356K, D399K | constant region | acid | |
| | HF; N297A, K409D, K439E, | Second heavy | DNA | 136 |
| | linker (G4SAAA), | chain (HF) | Amino | 137 |
| | aT04m0206-scFv | constant region | acid | |
| | L; wt | and | | |
| | | dsscFv region | | |
| | | Light chain (L) | DNA | 118 |
| | | constant region | Amino | 119 |
| | | | acid | |
| BR380 | HuAb1-hIgG1-N297A-Hn3- | First heavy | DNA | 158 |
| | HΔK-aT04m02.1_mv | chain (H) | Amino | 159 |
| | H; N297A, D356K, E357K, | constant region | acid | |
| | D399K, HΔK | Second heavy | DNA | 160 |
| | HF; N297A, K370E, K409D, | chain (HF) | Amino | 161 |
| | K439E, linker (G4SAAA), | constant region | acid | |
| | aT04m02-scFv | and | | |
| | L; wt | scFv region | | |
| | | Light chain (L) | DNA | 118 |
| | | constant region | Amino | 119 |
| | | | acid | |
| BR397 | HuAb1-hIgG1-N297A-Hn3- | First heavy | DNA | 158 |
| | HΔK-aT04m02.1_dsscFv | chain (H) | Amino | 159 |
| | H; N297A, D356K, E357K, | constant region | acid | |
| | D399K, HΔK | Second heavy | DNA | 162 |
| | HF; N297A, K370E, K409D, | chain (HF) | Amino | 163 |
| | K439E, linker (G4SAAA), | constant region | acid | |
| | aT04m02-dsscFv | and | | |
| | L; wt | dsscFv region | | |
| | | Light chain (L) | DNA | 118 |
| | | constant region | Amino | 119 |
| | | | acid | |

### Experimental Example 22: Production and Purification, and SDS-PAGE Analysis

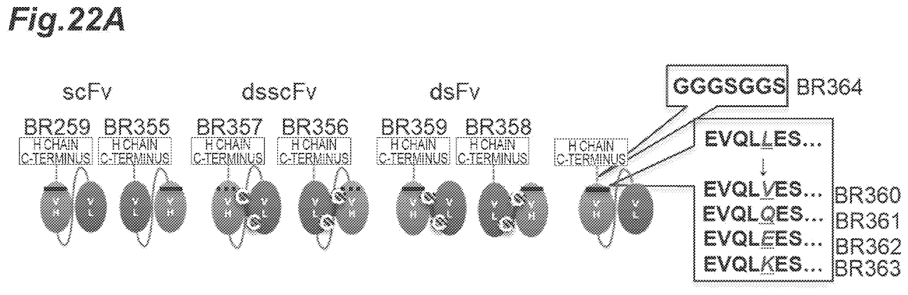

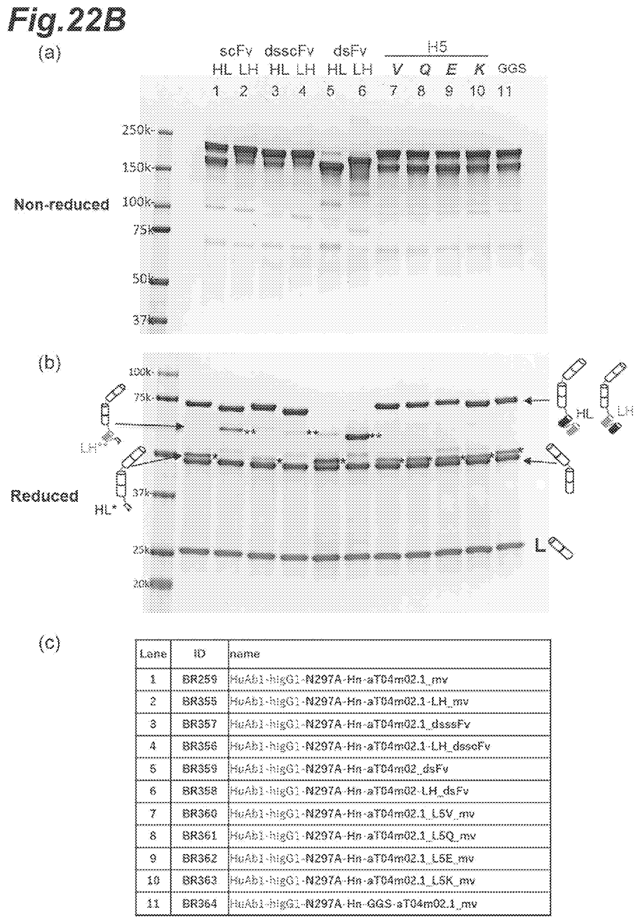

BR258, BR350, BR368, BR380, and BR397 were transiently produced according to the manufacturer's high-titer protocol using an ExpiCHO expression system (Thermo). The cell culture medium containing the secreted antibodies was collected 11 days after transfection. The clarified supernatant, by removing cells by centrifugation was purified using a MabSelect SuRe column (Cytiva, Inc.) or a KanCapA3G resin (KANEKA CORPORATION), and the eluate was buffer-exchanged with PBS using dialysis or a desalting column. BR259, BR355, BR357, BR356, BR359, BR358, BR360, BR361, BR362, BR363, and BR364 shown in FIG. 22A were transiently produced in a 96-well DeepWell plate according to the manufacturer's standard protocol using an ExpiCHO expression system (Thermo). The cell culture medium containing the secreted antibodies was collected 7 days after transfection. The clarified supernatant by removing cells by centrifugation was purified using a KanCapA3G resin (KANEKA CORPORATION), and the pH of the eluted fractions was adjusted by adding basic solution for pH adjustment. To prepare SDS-treated samples, the obtained diluted samples of the bispecific anti-human TfR/Aβ human IgG1 antibodies were mixed with SDS sample buffer at a volume ratio of 3:1. The mixture was heated at 99° C. for 5 minutes to prepare non-reduced samples for SDS-PAGE analysis. In addition, dithiothreitol was added to the SDS-treated samples to a final concentration of 50 mM, and mixture was heated at 99° C. for 5 minutes to prepare reduced samples. Each non-reduced and reduced samples, 1 μg, was applied on the gel, and the electrophoresis was performed at 200V for 45 minutes. The gel was stained with Bullet CBB Stain Lite (Nacalai tesque, Inc.), and destained with ultrapure water, and imaged using a ChemiDoc Touch MP Imaging System (Bio-Rad Laboratories, Inc.). As used herein, the term “truncation” refers to cleavage of the peptide bond between the leucine residues at positions 4 and 5 from the N-terminal side of the heavy chain variable region of aT04m02. The intensity of the bands indicated by asterisks (* or **) in the reduced SDS-PAGE scan image was evaluated as an index of truncation. Based on the results of SDS-PAGE shown in FIG. 22B, BR357 and BR356, both comprising dsscFv, exhibited the lowest levels of truncation.

### Experimental Example 23: Truncation Analysis by LC-MS

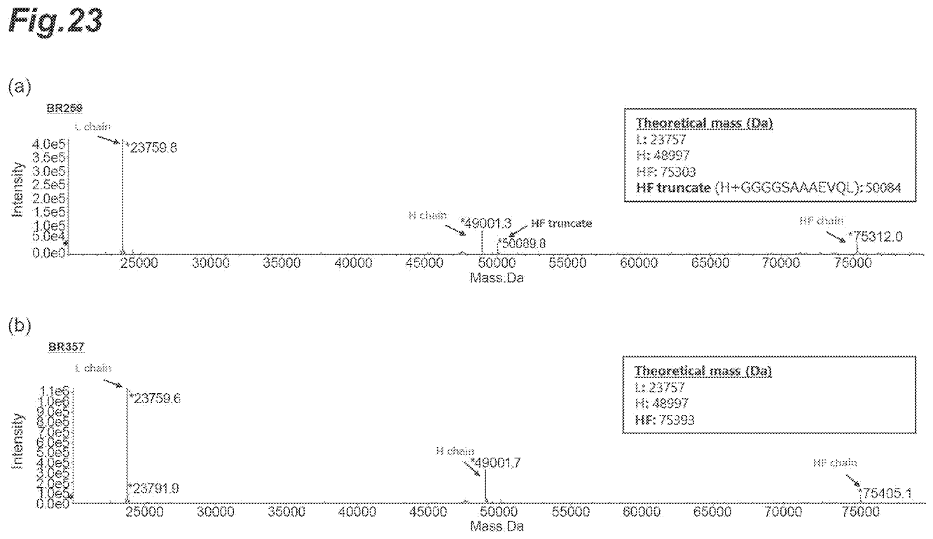

BR259 and BR357 obtained in Experimental Example 22 were diluted with D-PBS (−) and analyzed by LC-MS. The results of LC-MS are shown in FIG. 23. It was revealed from the results in FIG. 23 that truncation peaks were clearly confirmed in BR259 having scFv; however, truncation peaks were marginal in BR357 having dsscFv, resulting in the reduced amount generated.

### LC-MS Conditions

Measuring device: Triple TOF 6600+ (Sciex, Inc.)
Column: MassPrep On-Line desalting cartridges (Waters Corporation)
Column temperature: 80° C.
Mobile phase A: 0.1% formic acid-distilled water
Mobile phase B: 0.1% formic acid-acetonitrile
Flow rate: 0.3 mL/min
Injection volume: 1 μL
Detection: ESI (+), m/z=800-5000. Resolution=30000

### Experimental Example 24: Binding Evaluation to (Human and Monkey) TfR (SPR)

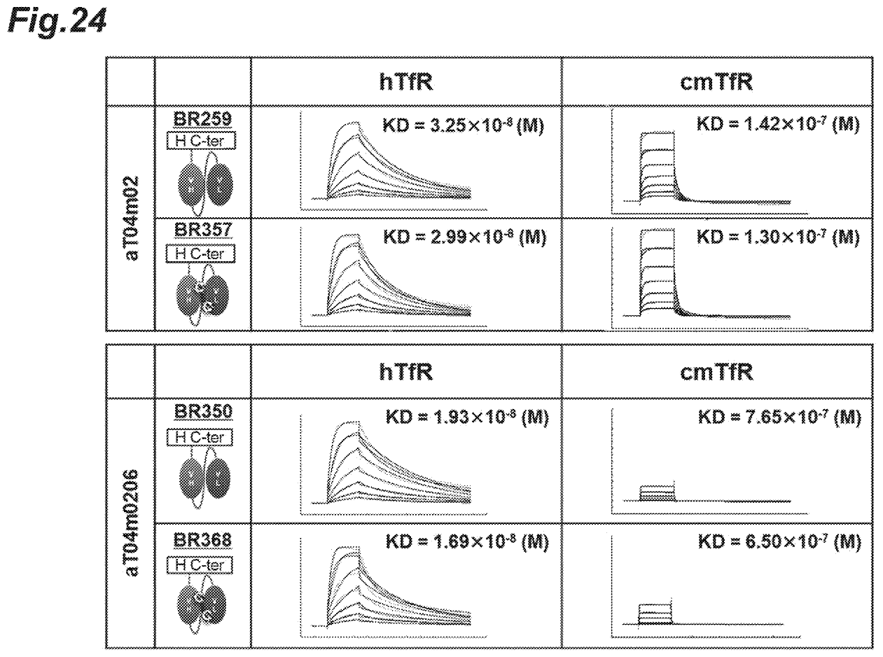

BR259, BR357, BR350, and BR368 were transiently produced according to the manufacturer's high-titer protocol using an ExpiCHO expression system (Thermo). The cell culture medium containing the antibodies was collected 11 days after transfection. The supernatant clarified by removing cells by centrifugation was purified using a KanCapA3G resin (KANEKA CORPORATION), and buffer-exchanged with PBS using a desalting column. The binding affinity of the obtained bispecific anti-human TfR/Aβ human IgG1 antibodies to human TfR and monkey TfR was measured using surface plasmon resonance (SPR method, Biacore T200, Cytiva, Inc.) using same method as in Experimental Example 18. The calculated KD values and binding curves are shown in FIG. 24. From FIG. 24, similar KD values were shown in BR259 and BR357 for both of human TfR and monkey TfR. Similarly, BR350 and BR368 exhibited comparable KD values for both human TfR and monkey TfR.

### Experimental Example 25: Construction of Stable Cell Line (SDS-PAGE and Titer)

### (1) Construction of Vector

DNA sequences encoding BR380 and BR397 were inserted into various pPV vectors of Gsquad vector System (Lonza). Table 16 shows each region constituting each antibody and the combinations of pPV vectors into which the respective regions were inserted. Based on the combinations described in Table 16, pPV vectors containing the respective antibody regions were fused with pPB vectors to construct expression vectors used for establishing stable cell lines expressing BR380 and BR397.

TABLE 16

| | | | DNA/ | |
|---|---|---|---|---|
| | | Antibody | Amino | pPV |
| ID | Antibody name | region | acid | vector |
| BR380 | HuAb1-hIgG1- | First heavy | DNA | pPV-B |
| | N297A-Hn3-HΔK- | chain (H) | | |
| | aT04m02.1 mv | Second heavy | DNA | pPV-A |
| | | chain (HF) | | |
| | | and scFv region | | |
| | (HF-H-L-L) | Light chain (L) | DNA | pPV-C |
| | | | | pPV-D |
| BR397 | HuAb1-hlgG1- | First heavy | DNA | pPV-B |
| | N297A-Hn3-HΔK- | chain (H) | | |
| | aT04m02.1 dsscFv | Second heavy | DNA | pPV-A |
| | | chain (HF) | | |
| | | and dsscFv | | |
| | | region | | |
| | (HF-H-L-L) | Light chain (L) | DNA | pPV-C |
| | | | | pPV-D |

### (2) Construction of Stable Cell Line

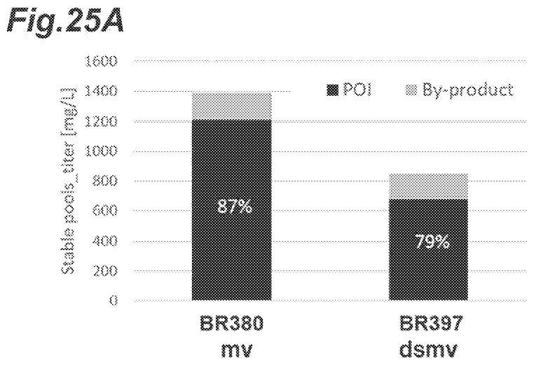

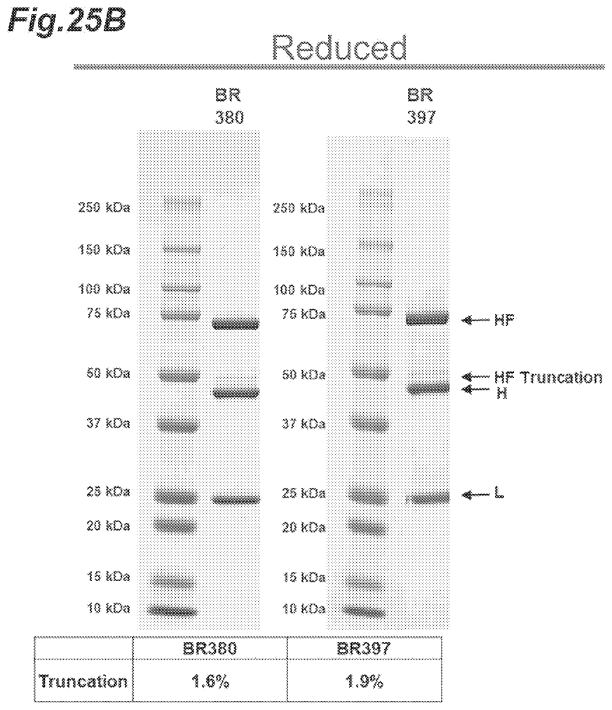

The produced expression vector was transfected into CHO cells by electroporation method, and selective culture was performed using a glutamine synthetase inhibitor, MSX (L-Methionine-DL-sulfoximine) in a 50 mL spin tube to establish a stable cell line. The cells were cultured with fed-batch mode for 14 days, and the antibody productivity including by-products was measured using Protein A Biosensor for Octet Red96e (Sartorius AG). The culture supernatant after removing the cells was affinity-purified using MabSelect SuRe (Cytiva, Inc.) column. The percentage of protein of interest (POI) was calculated from the results of SDS-PAGE analysis of purified products shown below. Hereinafter, the protein of interest and its percentage are described as “POI” and “POI purity”, respectively. It was suggested from the results of FIG. 25A that BR380 having scFv of a stable cell line has 1.8 times higher antibody productivity and higher POI purity by 8%, compared to BR397 having scFv including disulfide bond (dsscFV).

### (3) SDS-PAGE

BR380 or BR397 and SDS sample buffer were mixed at a volume ratio of 1:1, and heated at 70° C. for 10 minutes, and thereby non-reduced samples for SDS-PAGE analysis were prepared. Also, SDS sample buffer containing 10% 3-Mercapto-1, 2-propanediol and BR380 or BR397 were mixed at a volume ratio of 1:1, and heated at 70° C. for 10 minutes, and thereby reduced samples for SDS-PAGE analysis were prepared. These non-reduced and reduced samples were applied on the gel, and the electrophoresis was performed by applying current of 20 mA. After that, decolorization was performed with Coomassie staining, and decoloring solution (acetic acid:methanol:distilled water=1:1:8). At the end, the gel was scanned with a densitometer (Bio-Rad Laboratories, Inc., GS-900), and then purity evaluation was performed. The results of SDS-PAGE for the reduced samples are shown in FIG. 25B. From the results of FIG. 25B, the amount of truncation generated in BR380 and BR397 were both about 2%, which were equivalent.

### Experimental Example 26: Production of Monoclonal Antibodies (Transient Expression)

### (1) Construction of Modified Bispecific Anti-Human TfR/Aβ Human IgG1 Antibody Expression Gene Vector

Similarly to Experimental Example 1, in the vector expressing the first heavy chain containing the amino acid sequence of SEQ ID NO: 159 and the vector expressing the second heavy chain and scFv genes containing the amino acid sequence of SEQ ID NO: 161, a sequence in which the amino acid at residue 297 according to EU numbering was substituted with glycine (N297G) was synthesized. Accordingly, vectors expressing the first heavy chain gene (DNA sequence: SEQ ID NO: 164; amino acid sequence: SEQ ID NO: 165, in the constant region) and the second heavy chain and scFv gene (DNA sequence: SEQ ID NO: 166; amino acid sequence: SEQ ID NO: 167, in the constant region and scFv region) were constructed (BR381). Hereinafter, “N297G” in the antibody names in the tables refers to antibodies containing the N297G substitution in both the first and second heavy chains. Similarly, in the vector expressing the first heavy chain gene containing the amino acid sequence of SEQ ID NO: 125, the Hn3 mutation was introduced into the CH3 domain and the C-terminal lysine was removed. In the vector expressing the second heavy chain and scFv gene containing the amino acid sequence of SEQ ID NO: 127, the Hp3 mutation was introduced into the CH3 domain. Accordingly, vectors expressing the first heavy chain gene (DNA sequence: SEQ ID NO: 168; amino acid sequence: SEQ ID NO: 169, in the constant region) and the second heavy chain and scFv gene (DNA sequence: SEQ ID NO: 170; amino acid sequence: SEQ ID NO: 171, in the constant region and scFv region) were constructed (BR383). In addition, in the vectors expressing the first and second heavy chain genes containing the constant regions of SEQ ID NOs: 169 and 171, respectively, a sequence in which alanine at residue 297 was substituted with glycine was synthesized. Accordingly, vectors expressing the first heavy chain gene (DNA sequence: SEQ ID NO: 172; amino acid sequence: SEQ ID NO: 173, in the constant region) and the second heavy chain and scFv gene (DNA sequence: SEQ ID NO: 174; amino acid sequence: SEQ ID NO: 175, in the constant region and scFv region) were constructed (BR384). Further, in the vectors expressing the second heavy chain and scFv genes containing the amino acid sequences of SEQ ID NOs: 171 and 175, vectors expressing second heavy chain and scFv genes in which the amino acid sequence encoding aT04m02-scFv was replaced with the amino acid sequence encoding aT04m0206-scFv were also constructed (DNA sequences: SEQ ID NOs: 176 and 178; amino acid sequences: SEQ ID NOs: 177 and 179, in the constant region and scFv region) (BR387 and BR388). Note that the light chain constant region of the humanized anti-Aβ antibody is identical to the amino acid sequence of SEQ ID NO: 119, and the heavy chain constant region consists of the amino acid sequence of SEQ ID NO: 181, encoded by the DNA sequence of SEQ ID NO: 180 (BR220).

In the vector expressing the second heavy chain and scFv gene containing the amino acid sequence of SEQ ID NO: 127, an expression vector in which the amino acid sequence encoding aT04m02-scFv was replaced with the amino acid sequence encoding aT04m0206-scFv was constructed (DNA sequence: SEQ ID NO: 182; amino acid sequence: SEQ ID NO: 183, in the constant region and scFv region) (BR292). In addition, in the vectors expressing the first heavy chain genes containing the amino acid sequences of SEQ ID NOs: 169 and 159, expression vectors in which lysine was introduced at the C-terminus were constructed (DNA sequences: SEQ ID NOs: 184 and 186; amino acid sequences: SEQ ID NOs: 185 and 187, in the constant region) (BR317 and BR322).

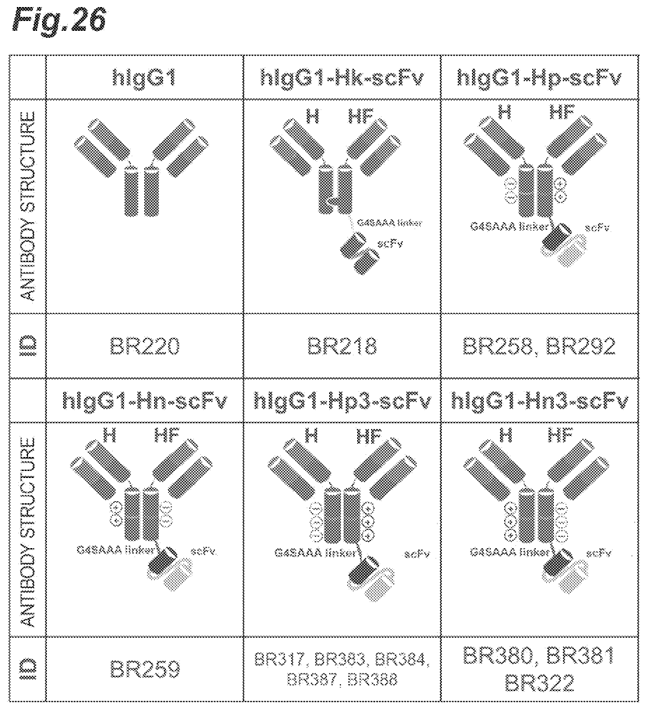

Schematic diagrams of respective antibodies, and the combinations of the first and second heavy chains and the light chains included in each antibody are shown in FIG. 26 and Table 17, respectively.

TABLE 17

| | | | DNA/ | SEQ |
|---|---|---|---|---|
| | | | Amino | ID |
| ID | Antibody name | Antibody region | acid | NO: |
| BR381 | HuAb1-hlgG1-N297G-Hn3- | First heavy | DNA | 164 |
| | HΔK-aT04m02.1_mv | chain (H) | Amino | 165 |
| | H; N297G, D356K, E357K, | constant region | acid | |
| | D399K, HΔK | Second heavy | DNA | 166 |
| | HF; N297G, K370E, | chain (HF) | Amino | 167 |
| | K409D, K439E, linker | constant region | acid | |
| | (G4SAAA), aT04m02-scFv | and | | |
| | L; wt | scFv region | | |
| | | Light chain (L) | DNA | 118 |
| | | constant region | Amino | 119 |
| | | | acid | |
| BR383 | HuAb1-hlgG1-N297A-Hp3- | First heavy | DNA | 168 |
| | HΔK-aT04m02.1_mv | chain (H) | Amino | 169 |
| | H; N297A, K370E, K409D, | constant region | acid | |
| | K439E, HΔK | Second heavy | DNA | 170 |
| | HF; N297A, D356K, | chain (HF) | Amino | 171 |
| | E357K, D399K, linker | constant region | acid | |
| | (G4SAAA), aT04m02-scFv | and | | |
| | L; wt | scFv region | | |
| | | Light chain (L) | DNA | 118 |
| | | constant region | Amino | 119 |
| | | | acid | |
| BR384 | HuAb1-hlgG1-N297G-Hp3- | First heavy | DNA | 172 |
| | HΔK-aT04m02.1_mv | chain (H) | Amino | 173 |
| | H; N297G, K370E, K409D, | constant region | acid | |
| | K439E, HΔK | Second heavy | DNA | 174 |
| | HF; N297G, D356K, | chain (HF) | Amino | 175 |
| | E357K, D399K, linker | constant region | acid | |
| | (G4SAAA), aT04m02-scFv | and | | |
| | L; wt | scFv region | | |
| | | Light chain (L) | DNA | 118 |
| | | constant region | Amino | 119 |
| | | | acid | |
| BR387 | HuAb1-hlgG1-N297A-Hp3- | First heavy | DNA | 168 |
| | HΔK-aT04m0206.1_mv | chain (H) | Amino | 169 |
| | H; N297A, K370E, K409D, | constant region | acid | |
| | K439E, HΔK | Second heavy | DNA | 176 |
| | HF; N297A, D356K, | chain (HF) | Amino | 177 |
| | E357K, D399K, linker | constant region | | |
| | (G4SAAA), aT04m0206- | and | | |
| | scFv | scFv region | | |
| | L; wt | Light chain (L) | DNA | 118 |
| | | constant region | Amino | 119 |
| | | | acid | |
| BR388 | HuAb1-hlgG1-N297G- | First heavy | DNA | 172 |
| | Hp3-HΔK- | chain (H) | Amino | 173 |
| | aT04m0206.1 mv | constant region | acid | |
| | H; N297G, K370E, K409D, | Second heavy | DNA | 178 |
| | K439E, HΔK | chain (HF) | Amino | 179 |
| | HF; N297G, D356K, | constant region | acid | |
| | E357K, D399K, linker | and | | |
| | (G4SAAA), aT04m0206- | scFv region | | |
| | scFv | Light chain (L) | DNA | 118 |
| | L; wt | constant region | Amino | 119 |
| | | | acid | |
| BR220 | HuAb1-hIgG1 | Heavy chain | DNA | 180 |
| | | (H) constant | Amino | 181 |
| | | region | acid | |
| | | Light chain (L) | DNA | 118 |
| | | constant region | Amino | 119 |
| | | | acid | |
| BR292 | HuAb1-hIgG1-N297A-Hp- | First heavy | DNA | 124 |
| | aT04m0206.1_mv | chain (H) | Amino | 125 |
| | H; N297A, K409D, K439E | constant region | acid | |
| | HF; N297A, D356K, | Second heavy | DNA | 182 |
| | D399K, linker (G4SAAA), | chain (HF) | | |
| | aT04m0206-scFv | constant region | | |
| | L; wt | and | Amino | 183 |
| | | scFv region | acid | |
| | | Light chain (L) | DNA | 118 |
| | | constant region | Amino | 119 |
| | | | acid | |
| BR317 | HuAb1-hlgG1-N297A- | First heavy | DNA | 184 |
| | Hp3-aT04m02.1_mv | chain (H) | Amino | 185 |
| | H; N297A, K370E, K409D, | constant region | acid | |
| | K439E | Second heavy | DNA | 170 |
| | HF; N297A, D356K, | chain (HF) | Amino | 171 |
| | E357K, D399K, linker | constant region | acid | |
| | (G4SAAA), aT04m02-scFv | and | | |
| | L; wt | scFv region | | |
| | | Light chain (L) | DNA | 118 |
| | | constant region | Amino | 119 |
| | | | acid | |
| BR322 | HuAb1-hIgG1-N297A- | First heavy | DNA | 186 |
| | Hn3-aT04m02.1 mv | chain (H) | Amino | 187 |
| | H; N297G, D356K, E357K, | constant region | acid | |
| | D399K | Second heavy | DNA | 160 |
| | HF; N297G, K370E, | chain (HF) | Amino | 161 |
| | K409D, K439E, linker | constant region | acid | |
| | (G4SAAA), aT04m02-scFv | and | | |
| | L; wt | scFv region | | |
| | | Light chain (L) | DNA | 118 |
| | | constant region | Amino | 119 |
| | | | acid | |

### (2) Production and Purification of Modified Bispecific Anti-Human TfR/Aβ Human IgG1 Antibodies

The anti-Aβ antibody BR220 and the bispecific anti-human TfR/Aβ human IgG1 antibodies BR218, BR258, BR259, BR380, BR383, and BR387 were transiently produced according to the manufacturer's high-titer protocol using an ExpiCHO expression system (Thermo). The cell culture medium containing the secreted antibodies was collected 11 days after transfection. The clarified supernatant, obtained by removing cells by centrifugation was purified using a MabSelect SuRe column (Cytiva, Inc.) or a KanCapA3G resin (KANEKA CORPORATION), and the eluate was buffer-exchanged with PBS by dialysis or a desalting column.

### Experimental Example 27: Binding Evaluation of Bispecific Anti-Human TfR/Humanized Aβ Antibody to Human TfR and Protofibril-Like Aβ (ELISA)

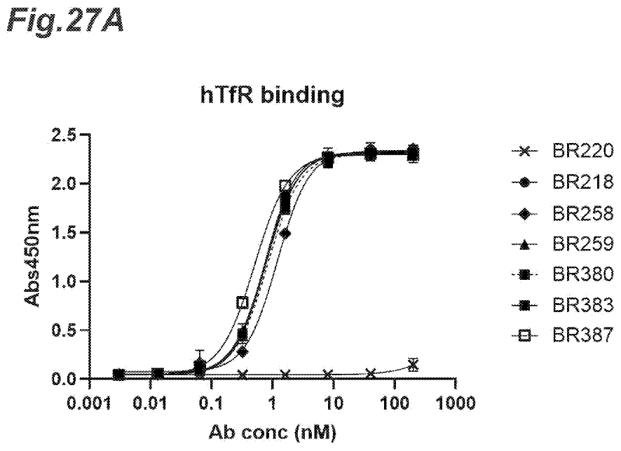

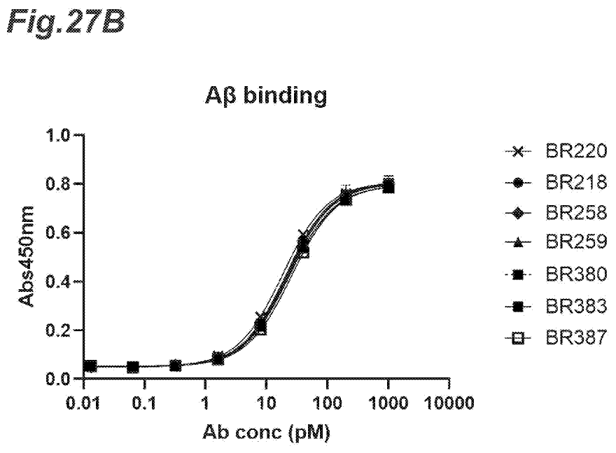

The binding of BR220, BR218, BR258, BR259, BR380, BR383, and BR387 to human TfR and protofibril-like Aβ was evaluated according to the ELISA procedure described in Experimental Example 13. The absorbance at 450 nm for various antibody concentrations is shown in FIG. 27A and FIG. 27B. From FIG. 27A, it was found that these bispecific anti-human TfR/humanized Aβ antibodies all exhibit equivalent binding properties to human TfR. Similarly, from FIG. 27B, it was found that these bispecific anti-human TfR/humanized Aβ antibodies all exhibit equivalent binding properties to protofibril-like Aβ.

### Experimental Example 28: Mouse PK

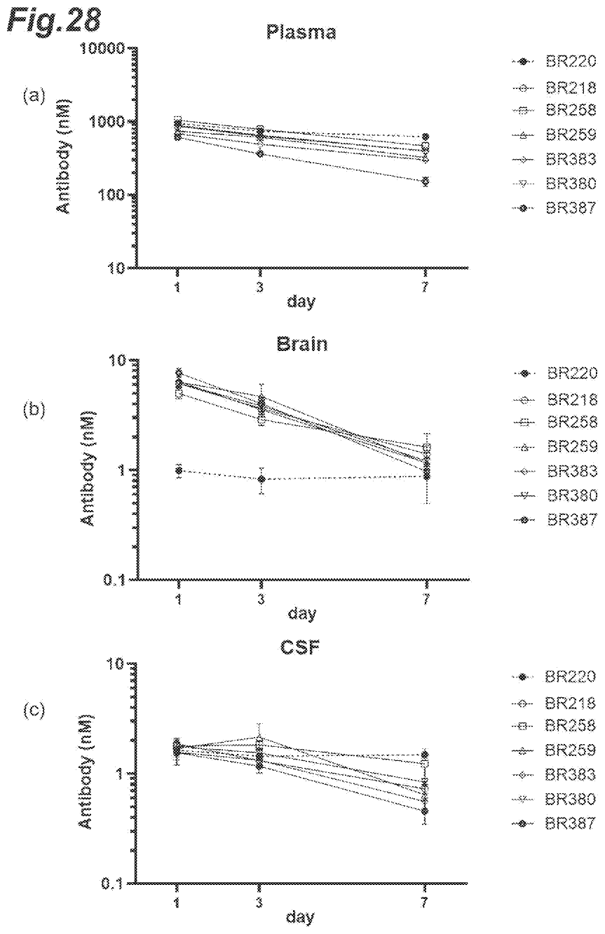

The anti-Aβ human IgG1 antibody BR220 and the bispecific anti-human TfR/Aβ human IgG1 antibodies BR218, BR258, BR259, BR380, BR383, and BR387 were administered as a single intravenous dose to male hTfR-ApD-KI (homo) mice at a dose of 20 mg/kg, calculated as an IgG equivalent amount. At 1, 3, and 7 days post-administration, blood was collected under anesthesia by facial venipuncture using heparin. After collecting CSF, exsanguination via incision of the right atrial appendage and transcardial perfusion with D-PBS (−) containing heparin and complete Protease Inhibitor Cocktail (Roche) were performed, and then, the brain was harvested. In order to measure the antibody in plasma, the blood collected using heparin was centrifuged at 1,200×g for 15 minutes at 4° C., and the supernatant was collected as plasma. The brain was cut in half, and frozen in liquid nitrogen. In order to measure the antibody concentration in the brain, 400 μL of complete Protease Inhibitor Cocktail-containing TBS was added into a collection tube of a BioMasher I (Nippi Inc.), and then, a filter tube was set, the right cerebral hemisphere was placed in it, and a pestle bar was inserted. After centrifugation at 14,000 rpm, 4° C. for 1 minute, the filter tube was removed, complete Protease Inhibitor Cocktail-containing TBS was added to bring the total amount including originally added 400 μL to 5 times the weight of the wet tissue, and pipetting was performed 10 times. NP-40 Alternative, 10% solution (Millipore) was added to the final concentration of 0.9%, and incubated at 4° C. for one hour while rotating. Centrifugation at 15,000 rpm, 4° C. for 1 minute was performed to collect the supernatant as a brain homogenate. The measurement of the antibody concentrations was performed by ELISA as described below. A donkey anti-human IgG-Fcγ chain-specific F(ab′)2 antibody (Jackson ImmunoResearch Inc.) was added to a MaxiSorp Plates (Thermo) and incubated overnight at 4° C. Washing was performed with 0.02% Tween20/PBS three times. After that, blocking was performed at room temperature for one hour by 1% Block Ace (DS Pharma Biomedical Co., Ltd.)/PBS, and then, the brain homogenate, plasma, or CSF diluted using 0.4% Block Ace/PBS were added and incubated at room temperature for 2 hours. After washing with 0.02% Tween20/PBS three times, an HRP-labeled goat anti-human IgG Fcγ chain-specific F(ab′)2 antibody (Jackson ImmunoResearch Inc.) was added and incubated at room temperature for one hour. After washing with 0.02% Tween20/PBS three times, TMB substrate solution (SeraCare) was added and incubated at room temperature for 30 minutes. An equivalent amount of 2N sulfuric acid was added to quench the reaction, and the absorbance at 450 nm and 650 nm was measured with a microplate reader SpectraMax ABS (Molecular Devices, LLC). Calibration curves were created based on the absorbance of standard samples at each concentration, and the antibody concentrations of the respective samples were determined. The results are shown in FIG. 28. It was indicated that the antibody concentrations in the brain one day after administration were remarkably higher in the bispecific antibodies BR218, BR258, BR259, BR380, BR383, and BR387 than BR220. For the antibody concentrations in the brain, plasma, and CSF at 1, 3, and 7 days post-administration, there were no marked differences between these six bispecific antibodies observed.

### Experimental Example 29: Construction of Stable Cell Line (SDS-PAGE and Titer)

### (1) Construction of Vector

Similarly to Experimental Example 25, DNA sequences encoding BR218, BR258, BR259, BR292, BR317, BR322, BR350, BR381, BR383, BR384, BR387, and BR388 were inserted into various pPV vectors. Table 18 shows each region constituting each antibody and the combinations of pPV vectors into which the respective regions were inserted. Based on the combinations described in Table 18, the pPV vectors containing the respective antibody regions were fused with pPB vectors to construct expression vectors used for establishing stable cell lines for each antibody.

TABLE 18

| | | | DNA/ | |
|---|---|---|---|---|
| | | | Amino | pPV |
| ID | Antibody name | Antibody region | acid | vector |
| BR218 | HuAb1-hIgG1-N297A-Hk- | First heavy | DNA | pPV-B |
| | aT04m02.1 mv | chain (H) | | |
| | (HF-H-L-L) | Second heavy | DNA | pPV-A |
| | | chain (HF) and | | |
| | | scFv region | | |
| | | Light chain (L) | DNA | pPV-C |
| | | | | pPV-D |
| BR258 | HuAb1-hIgG1-N297A-Hp- | First heavy | DNA | pPV-B |
| | aT04m02.1_mv | chain (H) | | |
| | (HF-H-L-L) | Second heavy | DNA | pPV-A |
| | | chain (HF) and | | |
| | | scFv region | | |
| | | Light chain (L) | DNA | pPV-C |
| | | | | pPV-D |
| BR259 | HuAb1-hIgG1-N297A-Hn- | First heavy | DNA | pPV-B |
| | aT04m02.1_mv | chain (H) | | |
| | (HF-H-L-L) | Second heavy | DNA | pPV-A |
| | | chain (HF) and | | |
| | | scFv region | | |
| | | Light chain (L) | DNA | pPV-C |
| | | | | pPV-D |
| BR292 | HuAb1-hIgG1-N297A-Hp- | First heavy | DNA | pPV-B |
| | aT04m0206.1_mv | chain (H) | | |
| | (HF-H-L-L) | Second heavy | DNA | pPV-A |
| | | chain (HF) and | | |
| | | scFv region | | |
| | | Light chain (L) | DNA | pPV-C |
| | | | | pPV-D |
| BR317 | HuAb1-hlgG1-N297A-Hp3- | First heavy | DNA | pPV-B |
| | aT04m02.1 mv | chain (H) | | |
| | (HF-H-L-L) | Second heavy | DNA | pPV-A |
| | | chain (HF) and | | |
| | | scFv region | | |
| | | Light chain (L) | DNA | pPV-C |
| | | | | pPV-D |
| BR322 | HuAb1-hlgG1-N297A-Hn3- | First heavy chain | DNA | pPV-B |
| | aT04m02.1 mv | (H) | | |
| | (HF-H-L-L) | Second heavy | DNA | pPV-A |
| | | chain (HF) and | | |
| | | scFv region | | |
| | | Light chain (L) | DNA | pPV-C |
| | | | | pPV-D |
| BR350 | HuAb1-hlgG1-N297A-Hn- | First heavy | DNA | pPV-B |
| | aT04m0206.1_mv | chain (H) | | |
| | (HF-H-L-L) | Second heavy | DNA | pPV-A |
| | | chain (HF) and | | |
| | | scFv region | | |
| | | Light chain (L) | DNA | pPV-C |
| | | | | pPV-D |
| BR381 | HuAb1-hIgG1-N297G-Hn3- | First heavy | DNA | pPV-B |
| | HΔK-aT04m02.1_mv | chain (H) | | |
| | (HF-H-L-L) | Second heavy | DNA | pPV-A |
| | | chain (HF) and | | |
| | | scFv region | | |
| | | Light chain (L) | DNA | pPV-C |
| | | | | pPV-D |
| BR383 | HuAb1-hlgG1-N297A-Hp3- | First heavy | DNA | pPV-B |
| | HΔK-aT04m02.1_mv | chain (H) | | |
| | (HF-H-L-L) | Second heavy | DNA | pPV-A |
| | | chain (HF) and | | |
| | | scFv region | | |
| | | Light chain (L) | DNA | pPV-C |
| | | | | pPV-D |
| BR384 | HuAb1-hlgG1-N297G-Hp3- | First heavy | DNA | pPV-B |
| | HΔK-aT04m02.1_mv | chain (H) | | |
| | (HF-H-L-L) | Second heavy | DNA | pPV-A |
| | | chain (HF) and | | |
| | | scFv region | | |
| | | Light chain (L) | DNA | pPV-C |
| | | | | pPV-D |
| BR387 | HuAb1-hIgG1-N297A-Hp3- | First heavy | DNA | pPV-B |
| | HΔK-aT04m0206.1_mv | chain (H) | | |
| | (HF-H-L-L) | Second heavy | DNA | pPV-A |
| | | chain (HF) and | | |
| | | scFv region | | |
| | | Light chain (L) | DNA | pPV-C |
| | | | | pPV-D |
| BR388 | HuAb1-hIgG1-N297G-Hp3- | First heavy | DNA | pPV-B |
| | HΔK-aT04m0206.1_mv | chain (H) | | |
| | (HF-H-L-L) | Second heavy | DNA | pPV-A |
| | | chain (HF) and | | |
| | | scFv region | | |
| | | Light chain (L) | DNA | pPV-C |
| | | | | pPV-D |

### (2) Construction of Stable Expression Cell Line

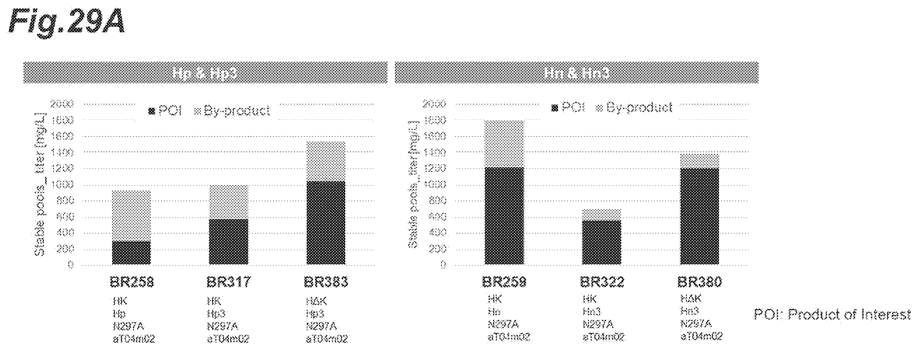

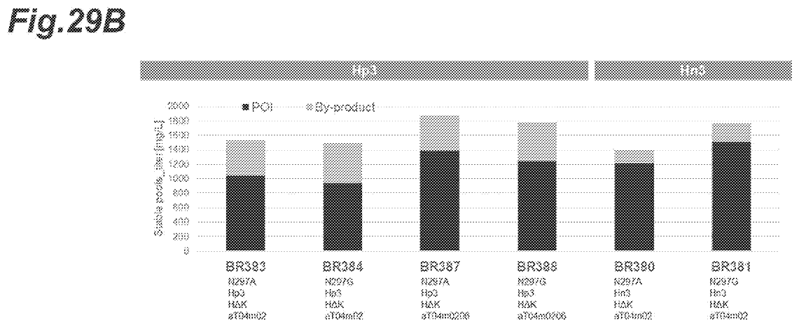

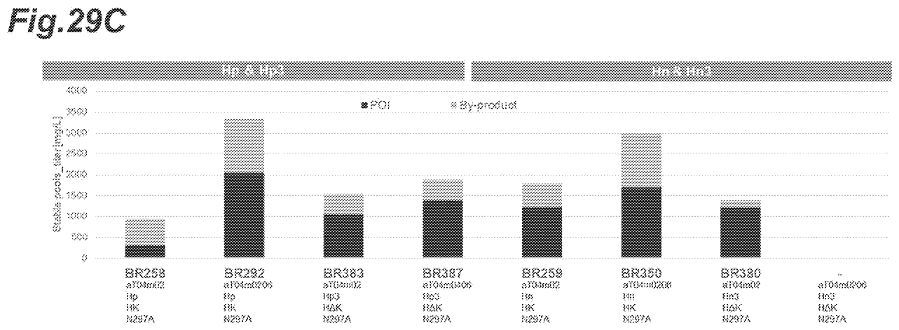

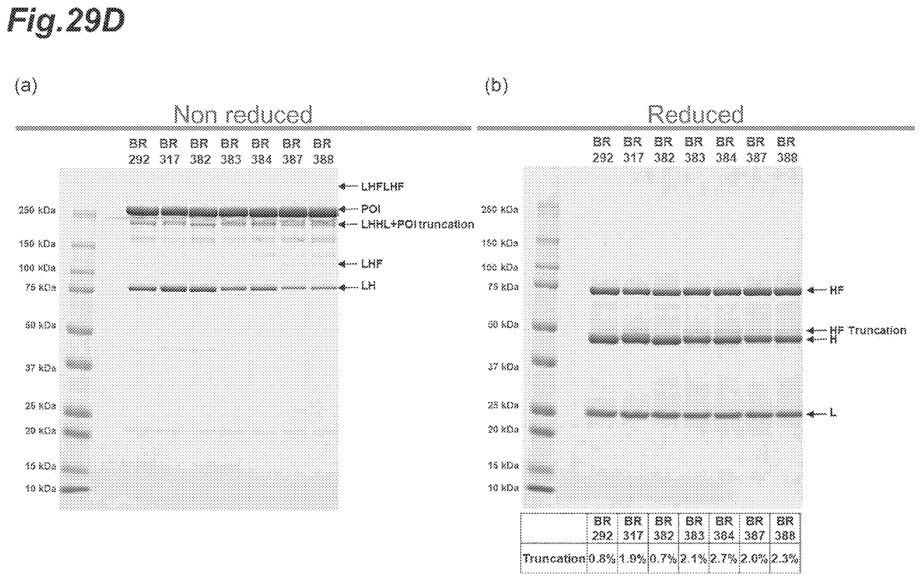

Stable cell lines were established by the method described in Experimental Example 25. The cells were cultured with fed-batch mode for 14 days, and the antibody productivity including by-products was measured using Protein A Biosensor for Octet Red96e (Sartorius AG). The culture supernatant was purified by using a MabSelect SuRe (Cytiva, Inc.) column. The antibody productivity in stable cell lines of respective antibodies are shown in FIG. 29A to FIG. 29C. There were no differences in the antibody productivity between N297A and N297G. On the other hand, when comparing BR317 and BR383, and BR322 and BR380, it was found that the antibody productivity in BR383 and BR380 each of which lacked lysine at the heavy chain C-terminus increased about 2-fold (FIG. 29A; “Hk” in the figure indicates the antibodies in which lysine was present at the C-terminus of the first heavy chain, and “HΔK” indicates the antibodies in which lysine was deleted at the C-terminus of the first heavy chain). Also, for scFv, the antibody productivity of the antibodies having aT04m0206 was higher than that of the antibodies having aT04m02 (FIG. 29B and FIG. 29C).

### (3) SDS-PAGE

The purified antibodies of BR292, BR317, BR382, BR383, BR384, BR387, and BR388 among the established stable cell lines, and SDS sample buffer were mixed at a volume ratio of 1:1, the mixture was heated at 70° C. for 10 minutes to prepare non-reduced samples for SDS-PAGE analysis. Also, SDS sample buffer containing 10% 3-Mercapto-1, 2-propanediol and the antibodies were mixed at a volume ratio of 1:1, and the mixture was heated at 70° C. for 10 minutes to prepare reduced samples for SDS-PAGE analysis. These non-reduced and reduced samples were applied on the gel, and the electrophoresis was performed by applying current of 20 mA. After that, decolorization was performed with Coomassie staining, and decoloring solution (acetic acid:methanol:distilled water=1:1:8). At the end, the gel was scanned with a densitometer (Bio-Rad, GS-900), and then purity evaluation was performed (FIG. 29D). From the results of FIG. 29D, the percentage of truncation was 0.7 to 2.7% of the total.

### Experimental Example 30: Separation and Purification

TSKgel SP-STAT (Tosoh) was used as cation exchange chromatography column, and the separation profile of byproducts and POI from BR259 was confirmed by gradient elution between 25 mM sodium acetate buffer (pH 5.0) and 25 mM sodium acetate/750 mM sodium chloride buffer (pH 5.0). A total of 11 fractions were aliquoted and then, buffer-exchanged and concentrated in D-PBS. Subsequently, analysis was performed by SDS-PAGE to confirm the separation status

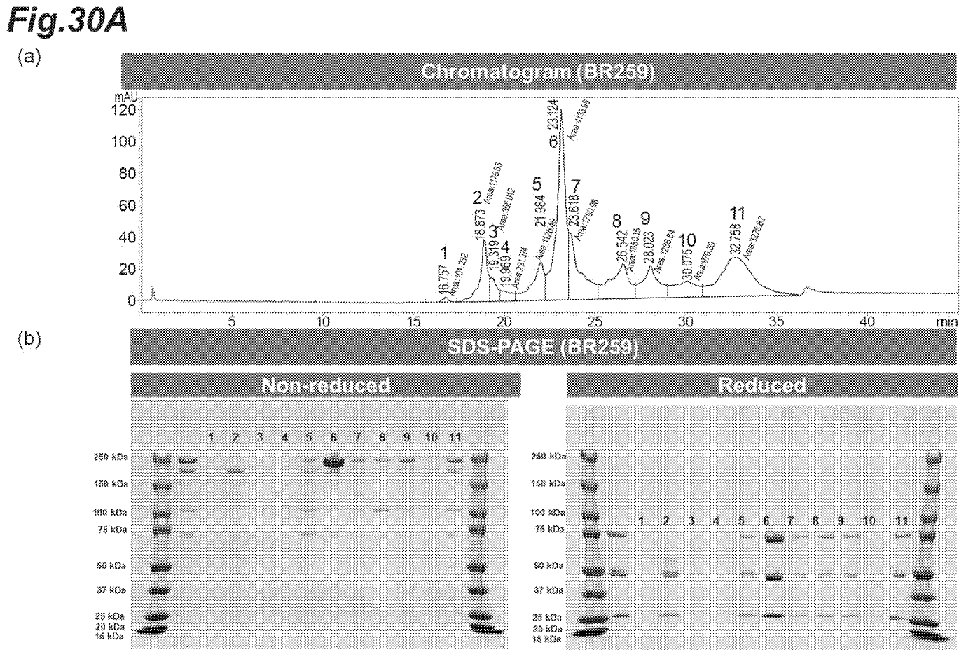

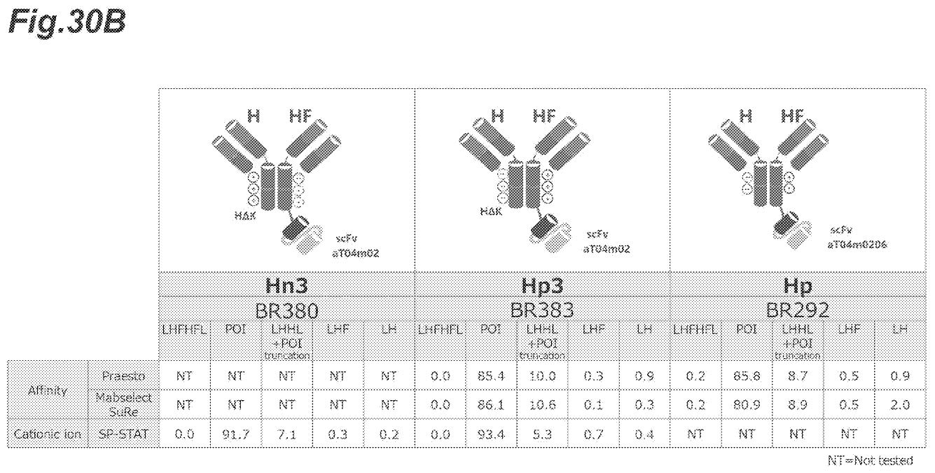

(FIG. 30A). It was confirmed from the results of FIG. 30A that 3 types of byproducts were co-eluted on the left shoulder in the eluted fractions of POI. For BR259 having the Hn mutation, since it was not possible to separate POI and byproducts by gradient elution using a HPLC column, it was revealed that the separation and removal of byproducts was difficult in an examination of use of a large column for purification process study.

### Conditions of Cation Exchange Chromatography

Measuring device: Agilent1200 (Agilent Technologies, Inc.)
Column: TSKgel SP-STAT (4.6 mm I.D.×10 cm, 7 μm) (Tosoh Bioscience, Inc.)
Column temperature: 25° C.
Mobile phase A: 25 mM acetic acid (pH 5.0)
Mobile phase B: 25 mM acetic acid/750 mM sodium chloride (pH 5.0)
Gradient conditions: 0 to 60% (0 to 30 min), 60 to 100% (30 to 35 min), 100 to 0% (35 to 35.01 min), 0% (35.01 to 45 min)
Flow rate: 1.0 mL/min
UV Detection: UV280 nm

For separation and purification by affinity chromatography, MabSelect SuRe (Cytiva, Inc.) or Praesto Jetted A50 High (Purolite Corporation) was used. Eluates of BR380, BR383, and BR292 in which the medium components were removed by a MabSelect SuRe (Cytiva, Inc.) column were applied on the above column, and then washed with 50 mM phosphate/50 mM sodium chloride (pH 7.0). After that, the byproducts and POI were separated by a stepwise gradient elution using 100 mM citrate buffer (pH 5.0), 100 mM citrate buffer (pH 4.0), and 100 mM citrate buffer (pH 3.0), or a stepwise elution by 100 mM citrate buffer (pH 5.0) and 100 mM citrate buffer (pH 3.6). Each fraction was analyzed by SDS-PAGE to confirm the separation status. FIG. 30B shows the results of quantification of the relative band intensities by image analysis of SDS-PAGE bands. It was revealed from the results that it is possible to separate POI and semi-antibodies in BR292 and BR383 having the Hp mutation or Hp3 mutation by a stepwise elution using citrate buffer, in particular. On the other hand, it was not possible to separate POI and semi-antibody in BR380 having the Hn3 mutation.

For the separation and purification by cation exchange chromatography, 5 mL SP-650M or CM-650M (Tosoh Bioscience, Inc.) was used. Eluates of BR380, BR383, and BR292 purified by a MabSelect SuRe (Cytiva, Inc.) column were diluted in 25 mM sodium acetate buffer (pH 5.5), applied on the column and washed, and then, the byproducts and POI were separated by gradient elution of 25 mM sodium acetate/750 mM sodium chloride buffer (pH 5.5). Each fraction was analyzed by SDS-PAGE to confirm the degree of separation. FIG. 30B shows the results of quantification of the relative band intensities by image analysis of SDS-PAGE bands. It was revealed from the results that it is possible to separate POI, and truncation and semi-antibodies in BR292 and BR383. It was revealed from the 2 types of separation and purification results that it is possible to ensure the POI purity of 90% or more in BR292 and BR383 having the Hp mutation or Hp3 mutation in combination of Protein A and cation exchange chromatography.

### Experimental Example 31: Evaluation of Binding Properties of Bispecific Anti-Human TfR/Humanized Aβ Antibody to Human TfR and Protofibril-Like Aβ (ELISA)

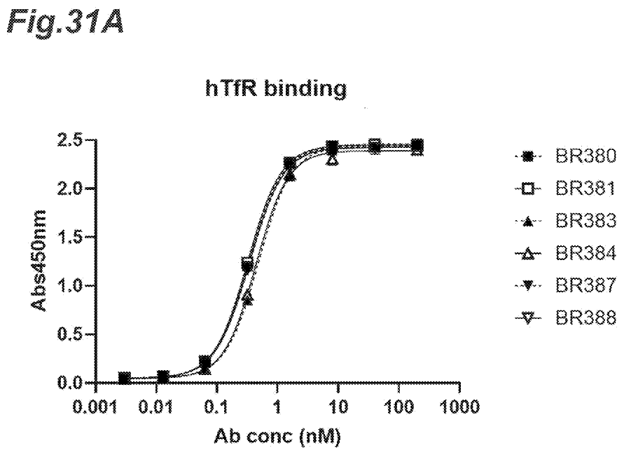

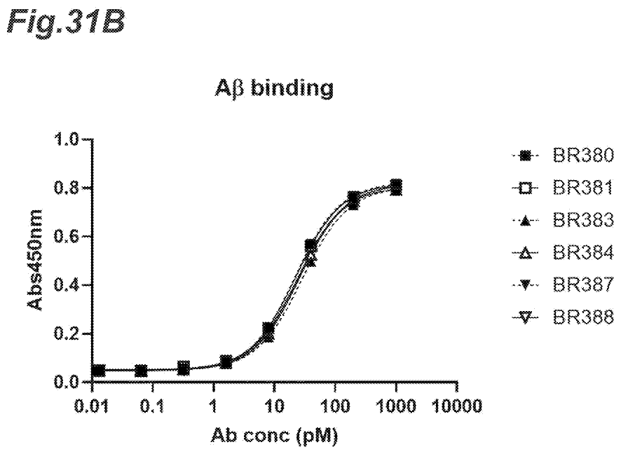

The evaluation of binding properties of BR380, BR381, BR383, BR384, BR387, and BR388 to human TfR and protofibril-like Aβ was performed according to the ELISA steps described in Experimental Example 13. The absorbance of each antibody concentration at 450 nm is shown in FIG. 31A and FIG. 31B. As shown in FIG. 31A, these bispecific anti-human TfR/humanized Aβ antibodies all have equivalent binding properties to human TfR. Also, as shown in FIG. 31B, these bispecific anti-human TfR/humanized Aβ antibodies all have equivalent binding properties to protofibril-like Aβ.

### Experimental Example 32: Evaluation of Binding Properties of Bispecific Anti-Human TfR/Humanized Aβ Antibody to Human and Mouse C1q (ELISA)

### (1) Evaluation of Binding Properties to Human C1q

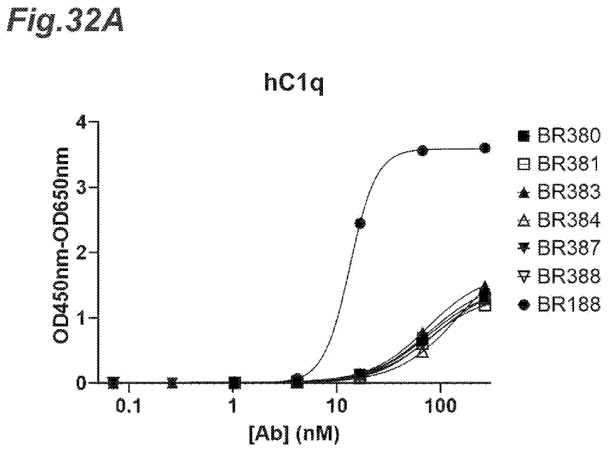

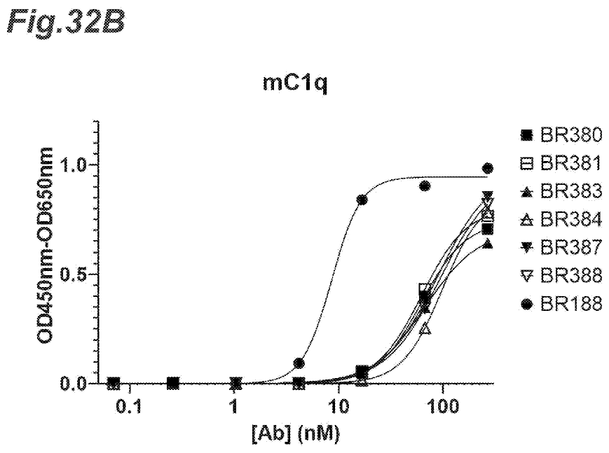

ELISA for the binding of BR380, BR381, BR383, BR384, BR387, and BR388 to human C1q was performed according to the procedure described in Experimental Example 19. The absorbance values at 450 nm, corrected by subtraction of background at 650 nm, are plotted in FIG. 32A. As shown in FIG. 32A, bispecific antibodies having N297A or N297G confirmed to have suppressed binding to human C1q compared with BR188.

### (2) Evaluation of Binding Properties to Mouse C1q

The binding of BR380, BR381, BR383, BR384, BR387, and BR388 to mouse C1q was evaluated by ELISA below. Antibody solutions prepared as dilution series were added to Immuno plate MaxiSorp (Thermo) and incubated overnight at 4° C. to immobilize the antibodies on the plate. After removing the antibody solutions, 0.5% BSA/0.4% I-Block (Thermo)/0.05% Tween20/PBS was added, and blocking was performed at room temperature for one hour. After washing the plate with 0.05% Tween20/PBS three times, mouse C1q (ProSpec-Tany TechnoGene Ltd.) was added to each well and incubated at room temperature for 1.5 hours. After washing with 0.05% Tween20/PBS three times, bound mouse C1q was detected using biotin-labeled anti-C1q antibody (Hycult Biotech), followed by HRP-labeled streptavidin. TMBZ (SeraCare) was used as the chromogenic substrate, and the reaction was terminated by addition of sulfuric acid. Absorbance values at 450 nm, corrected by subtraction of background at 650 nm, are plotted in FIG. 32B. As shown in FIG. 32B, bispecific antibodies having N297A or N297G exhibited reduced binding to mouse C1q compared with BR188.

### Experimental Example 33: Acid Resistance Evaluation

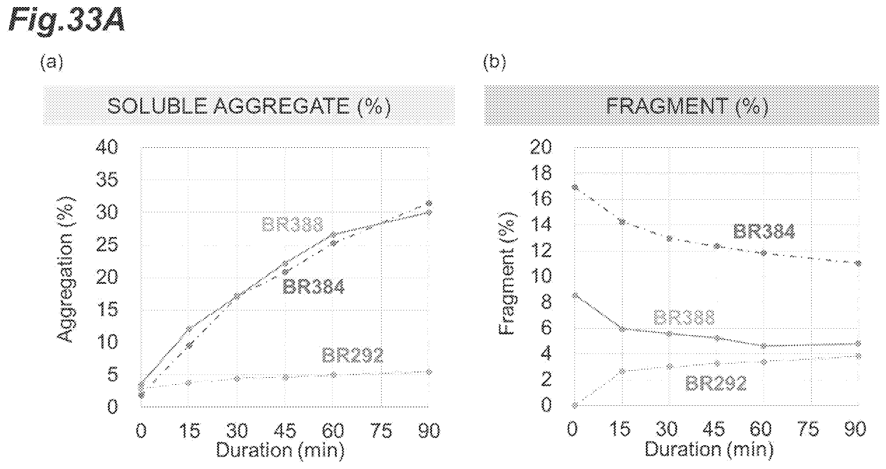

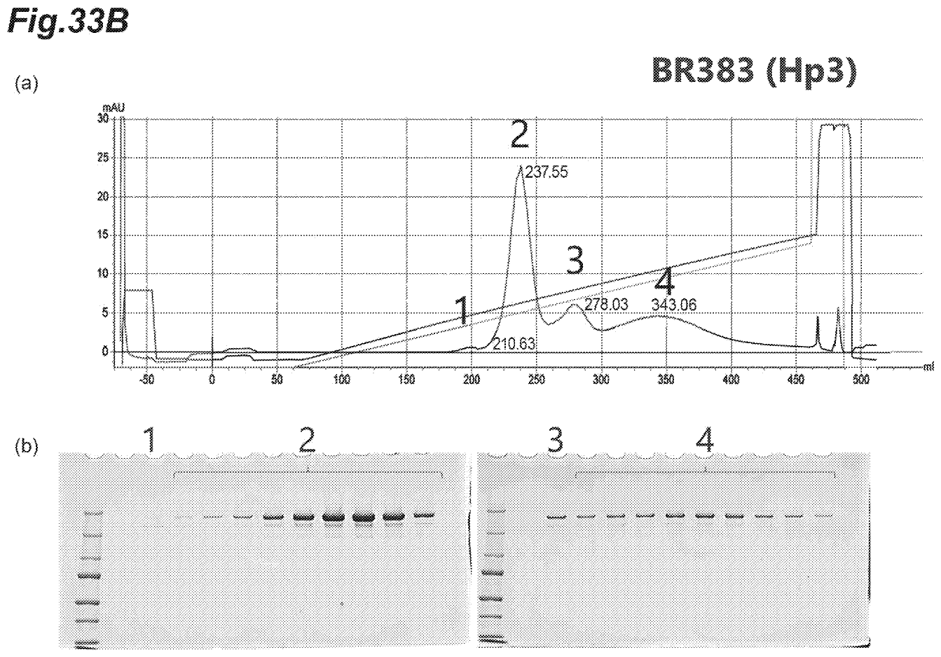

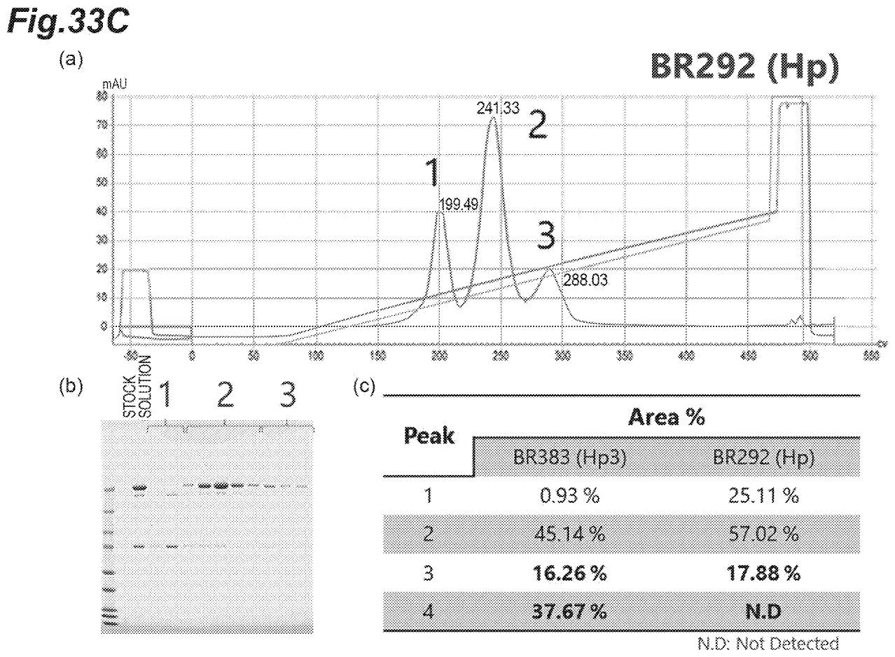

To solutions of BR292, BR384, and BR388 dissolved in 25 mM L-histidine (pH 5.5), 2M acetic acid was gradually added to adjust the pH to 3.6. After that, incubation was performed in a thermostat bath of 25° C. for 90 minutes. During the incubation, sampling was performed every 15 minutes, and 2M Tris (pH 9.0) was added in a small amount to adjust the pH to about 6.0. After that, the relative amounts of high-molecular fractions, main fraction, and low-molecular fractions were evaluated in area percentage by size-exclusion chromatography (FIG. 33A). As a result, it was confirmed that the increase in high-molecular fractions is only marginal in BR292 having the Hp mutation, while those in BR384 and BR388 having the Hp3 mutation markedly increase. Accordingly, it was revealed that antibodies having the Hp mutation are far more excellent in acid resistance than antibodies having the Hp3 mutation, and even in the process of virus inactivation due to acidic pH exposure in a normal antibody purification process, they are stably present.

### Size-Exclusion Chromatography, Condition 1

Measuring device: Nexera XSi (Shimadzu Corporation)
Column: TSKgel G3000SWXL (7.8 mm I.D.×30 cm, 5 μm) (Tosoh Bioscience, Inc.)
Column temperature: 25° C.
Mobile phase A: 50 mM phosphate 300 mM sodium chloride 1 mg/mL 8-Anilino-1-naphthalen sulfonic acid (pH 7.0)
Flow rate: 0.5 mL/min
UV Detection: UV280 nm
Fluorescence detector: EX=380 nm, Em=510 nm

### Size-Exclusion Chromatography, Condition 2

Measuring device: Nexera XSi (Shimadzu Corporation)
Column: TSKgel G3000SWXL (7.8 mm I.D.×30 cm, 5 μm) (Tosoh Bioscience, Inc.)
Column temperature: 25° C.
Mobile phase A: 50 mM phosphate 250 mM L-arginine (pH 7.0)
Flow rate: 0.5 mL/min
UV Detection: UV280 nm

For separation and purification by cation exchange chromatography, 5 mL SP-650M (Tosoh) was used. Eluates purified by a MabSelect SuRe (Cytiva, Inc.) column were diluted in 25 mM sodium acetate buffer (pH 5.5), applied on the column and washed, and then, the byproducts and POI were separated by gradient elution of 25 mM sodium acetate/750 mM sodium chloride buffer (pH 5.5). Each fraction was analyzed by SDS-PAGE to confirm the degree of the separation (FIG. 33B, FIG. 33C). It was found from confirming reversibility of POI oligomer by HPLC analysis and the results from SDS-PAGE analysis that the peak 3 of BR292 and BR383 is a reversible oligomer of POI, and the peak 4 is an oligomer of POI. Therefore, it was found from the results of relative comparison of monomer/oligomer of BR292 and BR383 that BR292 having the Hp mutation is higher in the ratio of monomer and more stable than BR383 having the Hp3 mutation.

### Experimental Example 34: Production of Monoclonal Antibodies (Transient Expression)

### (1) Construction of Bivalent-Type Bispecific Anti-Human TfR/Aβ Human IgG1 Antibody Expression Vector

In the vector expressing the heavy chain gene containing the amino acid sequence of SEQ ID NO: 181, sequences introducing the N297A mutation was synthesized (DNA sequence: SEQ ID NO: 202; amino acid sequence: SEQ ID NO: 203, in the constant region) (BR467). Further, aT04m02-scFv or aT04m0206-scFv was inserted via a G4S linker into the C-terminus of the human IgG1 heavy chain constant region. Accordingly, vectors expressing heavy chain genes having aT04m02-scFv or aT04m0206-scFv at the C-terminus were constructed (DNA sequences: SEQ ID NOs: 204 and 206; amino acid sequences: SEQ ID NOs: 205 and 207, in the constant region and scFv region) (BR441 and BR468). Similarly, in the vector expressing the heavy chain gene containing the amino acid sequence of SEQ ID NO: 203, the C-terminal lysine was removed. In the vector expressing the light chain gene containing the amino acid sequence of SEQ ID NO: 119, aT04m02-scFv or aT04m0206-scFv was inserted into the C-terminus via a linker sequence. Accordingly, vectors expressing the HΔK-type heavy chain gene (DNA sequence: SEQ ID NO: 208; amino acid sequence: SEQ ID NO: 209, in the constant region) and the light chain gene having aT04m02-scFv or aT04m0206-scFv at the C-terminus (DNA sequences: SEQ ID NOs: 210 and 212; amino acid sequences: SEQ ID NOs: 211 and 213, in the constant region and scFv region) were constructed (BR443 and BR445). Hereinafter, in the antibody names in the tables, antibodies having scFv at the C-terminus of both heavy chain constant regions are referred to as “HCbv”, and antibodies having scFv at the C-terminus of the light chain constant region are referred to as “LCbv.”

Similarly to Experimental Example 21, in the vectors expressing the heavy chain and scFv containing the amino acid sequence of SEQ ID NO: 205, the heavy chain and scFv containing the amino acid sequence of SEQ ID NO: 207, the light chain and scFv containing the amino acid sequence of SEQ ID NO: 211, or the light chain and scFv containing the amino acid sequence of SEQ ID NO: 213, glycine at the ninth residue from the N-terminus of HFR2 (heavy chain framework region 2) and glycine at the third residue from the N-terminus of LFR4 (light chain framework region 4), defined by Kabat numbering, were substituted with cysteine in aT04m02-scFv or aT04m0206-scFv (aT04m02-dsscFv or aT04m0206-dsscFv). Accordingly, vectors expressing new heavy chain and dsscFv genes (DNA sequences: SEQ ID NOs: 214 and 216; amino acid sequences: SEQ ID NOs: 215 and 217, in the constant region and dsscFv region) and new light chain and dsscFv genes (DNA sequences: SEQ ID NOs: 218 and 220; amino acid sequences: SEQ ID NOs: 219 and 221, in the constant region and dsscFv region) were constructed (BR442, BR469, BR444, and BR446). Hereinafter, antibodies having scFv (dsscFv) containing a disulfide bond formed by the two cysteine substitutions at the C-terminus of the heavy chain or light chain constant region are referred to as “HCdsbv” or “LCdsbv.”

In the vectors expressing the heavy chain and scFv containing the amino acid sequence of SEQ ID NO: 205, the heavy chain and scFv containing the amino acid sequence of SEQ ID NO: 207, the heavy chain containing the amino acid sequence of SEQ ID NO: 209, the light chain and scFv containing the amino acid sequence of SEQ ID NO: 211, or the light chain and scFv containing the amino acid sequence of SEQ ID NO: 213, the N297G mutation was introduced into the heavy chain gene and, further tryptophan at the seventh residue from the N-terminus of HCDR3 (defined by Kabat numbering) was substituted with serine (W105S) in the aT04m02-scFv or aT04m0206-scFv sequences connected to the C-terminus of the heavy or light chain via linker sequence. Accordingly, vectors expressing heavy chain genes (DNA sequence: SEQ ID NO: 226; amino acid sequence: SEQ ID NO: 227, in the constant region), new heavy chain genes (DNA sequences: SEQ ID NOs: 222 and 224; amino acid sequences: SEQ ID NOs: 223 and 225, in the constant region and scFv region), and light chain genes (DNA sequences: SEQ ID NOs: 228 and 230; amino acid sequences: SEQ ID NOs: 229 and 231, in the constant region and scFv region) were constructed (BR663, BR665, BR664, and BR666). Hereinafter, aT04m02-scFv or aT04m0206-scFv containing the W105S substitution is referred to as “aT04m02-W105S” or “aT04m0206-W105S.”

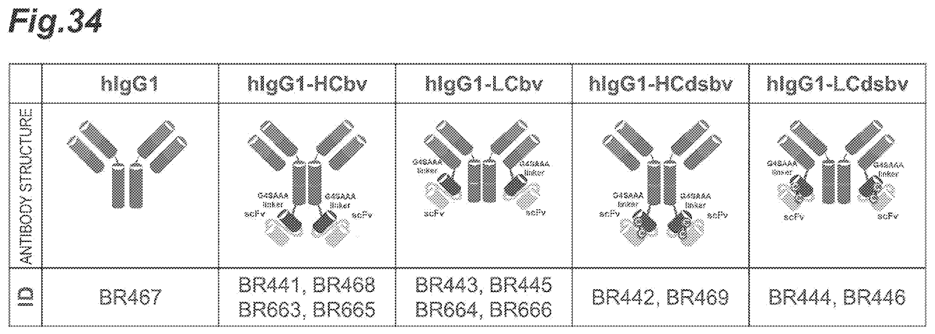

Schematic diagrams of respective antibodies and the combinations of the first and second heavy chains and the light chains included in each antibody are shown in FIG. 34 and Table 19, respectively.

TABLE 19

| | | | DNA/ | SEQ |
|---|---|---|---|---|
| | | | Amino | ID |
| ID | Antibody name | Antibody region | acid | NO: |
| BR467 | HuAb1-hIgG1-N297A | Heavy chain (H) | DNA | 202 |
| | H; N297A | constant region | Amino | 203 |
| | L; wt | | acid | |
| | | Light chain (L) | DNA | 118 |
| | | constant region | Amino | 119 |
| | | | acid | |
| BR441 | HuAb1-hIgG1-N297A- | Heavy chain (H) | DNA | 204 |
| | aT04m02.1.HCbv | constant region | Amino | 205 |
| | H; N297A, linker | and scFv region | acid | |
| | (G4SAAA), | Light chain (L) | DNA | 118 |
| | aT04m02-scFv | constant region | Amino | 119 |
| | L; wt | | acid | |
| BR468 | HuAb1-hIgG1-N297A- | Heavy chain (H) | DNA | 206 |
| | aT04m0206.1 HCbv | constant region | Amino | 207 |
| | H; N297A, linker | and scFv region | acid | |
| | (G4SAAA), | Light chain (L) | DNA | 118 |
| | aT04m0206-scFv | constant region | Amino | 119 |
| | L; wt | | acid | |
| BR443 | HuAb1-hIgG1-N297A- | Heavy chain (H) | DNA | 208 |
| | aT04m02.1 LCbv | constant region | Amino | 209 |
| | H; N297A | | acid | |
| | L; wt, linker | Light chain (L) | DNA | 210 |
| | (G4SAAA), | constant region | Amino | 211 |
| | aT04m02-scFv | and scFv region | acid | |
| BR445 | HuAb1-hlg1-N297A- | Heavy chain (H) | DNA | 208 |
| | aT04m0206.1 LCbv | constant region | Amino | 209 |
| | H; N297A | | acid | |
| | L; wt, linker | Light chain (L) | DNA | 212 |
| | (G4SAAA), | constant region | Amino | 213 |
| | aT04m0206-scFv | ands cFv region | acid | |
| BR442 | HuAb1-hlgG1-N297A- | Heavy chain (H) | DNA | 214 |
| | aT04m02.1 HCdsbv | constant region | Amino | 215 |
| | H; N297A, linker | and dsscFv | acid | |
| | (G4SAAA), | region | | |
| | aT04m02-dsscFv | Light chain (L) | DNA | 118 |
| | L; wt | constant region | Amino | 119 |
| | | | acid | |
| BR469 | HuAb1-hIgG1-N297A- | Heavy chain (H) | DNA | 216 |
| | aT04m0206.1_HCdsbv | constant region | Amino | 217 |
| | H; N297A, linker | and dsscFv | acid | |
| | (G4SAAA), | region | | |
| | aT04m0206-dsscFv | Light chain (L) | DNA | 118 |
| | L; wt | constant region | Amino | 119 |
| | | | acid | |
| BR444 | HuAb1-hIgG1-N297A- | Heavy chain (H) | DNA | 208 |
| | aT04m02.1 LCdsbv | constant region | Amino | 209 |
| | H; N297A | | acid | |
| | L; wt, linker | Light chain (L) | DNA | 218 |
| | (G4SAAA), | constant region | Amino | 219 |
| | aT04m02-dsscFv | and dsscFv | acid | |
| | | region | | |
| BR446 | HuAb1-hlg1-N297A- | Heavy chain (H) | DNA | 208 |
| | aT04m0206.1 LCdsbv | constant region | Amino | 209 |
| | H; N297A | | acid | |
| | L; wt, linker | Light chain (L) | DNA | 220 |
| | (G4SAAA), | constant region | Amino | 221 |
| | aT04m0206-dsscFv | and dsscFv | acid | |
| | | region | | |
| BR663 | HuAb1-hIgG1-N297G- | Heavy chain (H) | DNA | 222 |
| | aT04m02-W105S HCbv | constant region | Amino | 223 |
| | | and scFv region | acid | |
| | H; N297G, linker | | DNA | 118 |
| | (G4SAAA), aT04m02- | | | |
| | W105S-scFv | Light chain (L) | Amino | 119 |
| | L; wt | constant region | acid | |
| BR665 | HuAb1-hlgG1-N297G- | Heavy chain (H) | DNA | 224 |
| | aT04m0206-W105S | constant region | Amino | 225 |
| | HCbv | and scFv region | acid | |
| | H; N297G, linker | | DNA | 118 |
| | (G4SAAA), | Light chain (L) | Amino | 119 |
| | aT04m0206- | constant region | acid | |
| | W105S-scFv | | | |
| | L; wt | | | |
| BR664 | HuAb1-hlgG1-N297G- | Heavy chain (H) | DNA | 226 |
| | aT04m02-W105S LCbv | constant region | Amino | 227 |
| | H; N297G | | acid | |
| | L; wt, linker | Light chain (L) | DNA | 228 |
| | (G4SAAA), | constant region | Amino | 229 |
| | aT04m02-W105S-scFv | and scFv region | acid | |
| BR666 | HuAb1-hlgG1-N297G- | Heavy chain (H) | DNA | 226 |
| | aT04m0206-W105S | constant region | Amino | 227 |
| | LCbv | | acid | |
| | H; N297G | Light chain (L) | DNA | 230 |
| | L; wt, linker | constant region | Amino | 231 |
| | (G4SAAA), | and scFv region | acid | |
| | aT04m0206-W105S- | | | |
| | scFv | | | |

### (2) Production and Purification of Bispecific Anti-Human TfR/Aβ Human IgG1 Antibodies

The bivalent-type bispecific anti-human TfR/Aβ human IgG1 antibodies described in FIG. 34 were transiently produced according to the manufacturer's high-titer protocol using an ExpiCHO expression system (Thermo). The cell culture medium containing the secreted antibodies was collected 8 to 9 days after transfection. The clarified supernatant, obtained by removing cells by centrifugation, was purified using a MabSelect SuRe column (Cytiva, Inc.) or a KanCapA3G resin (KANEKA CORPORATION), and the eluate was buffer exchanged with PBS by dialysis or a desalting column.

### Experimental Example 35: Binding Evaluation to Antigen (Aβ and hTfR SPR)

### (1) Aβ SPR

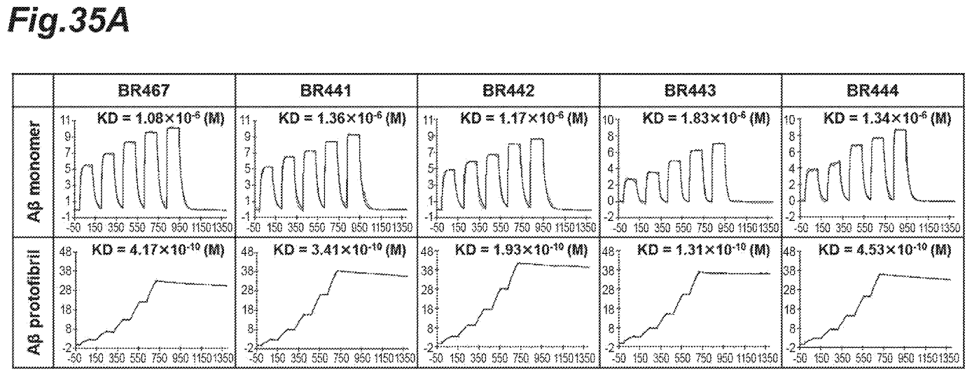

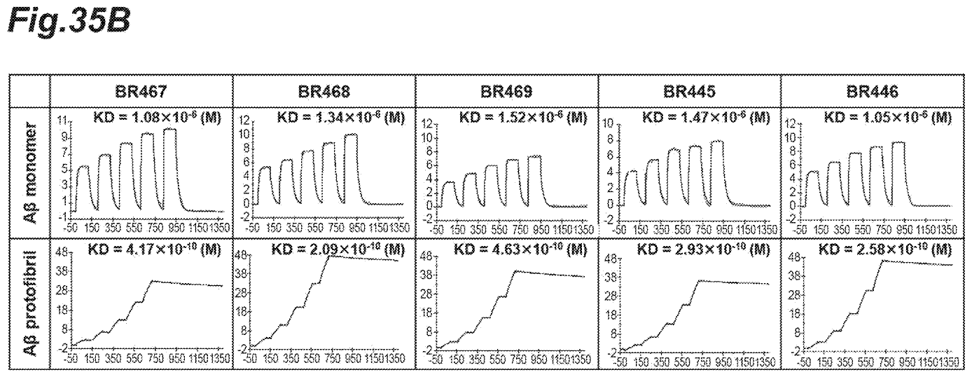

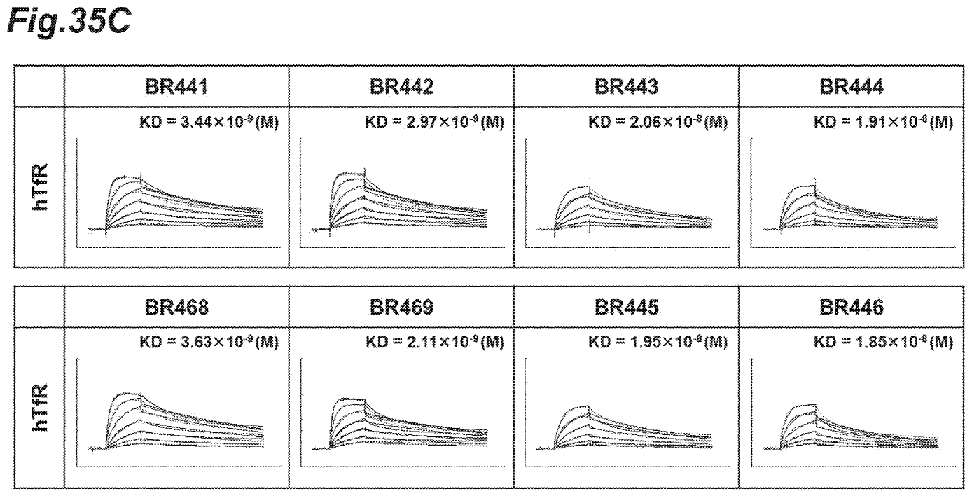

The binding affinity of the bispecific anti-human TfR/Aβ human IgG1 antibody prepared in Experimental Example 34 to monomer Aβ and protofibril was measured by SPR according to the procedure described in Experimental Example 18. The calculated KD values and binding response curves are shown in FIGS. 35A and 35B. As shown in FIGS. 35A and 35B, all the bispecific antibodies exhibit equivalent binding affinity to monomer Aβ and protofibril.

### (2) TfR SPR

The binding affinity of the bispecific anti-human TfR/Aβ human IgG1 antibody prepared in Experimental Example 34 to human TfR was measured by SPR according to the procedure described in Experimental Example 18. The calculated KD values and binding response curves are shown in FIG. 35C. BR441, BR442, BR468, and BR469 having 2 scFvs at the H-chain C-terminus exhibited KD values about 10 times stronger (lower KD value) than BR443, BR444, BR445, and BR446 having 2 scFvs at the L-chain C-terminus.

### Experimental Example 36: Mouse PK (hTfR-ApD-KI Mouse)

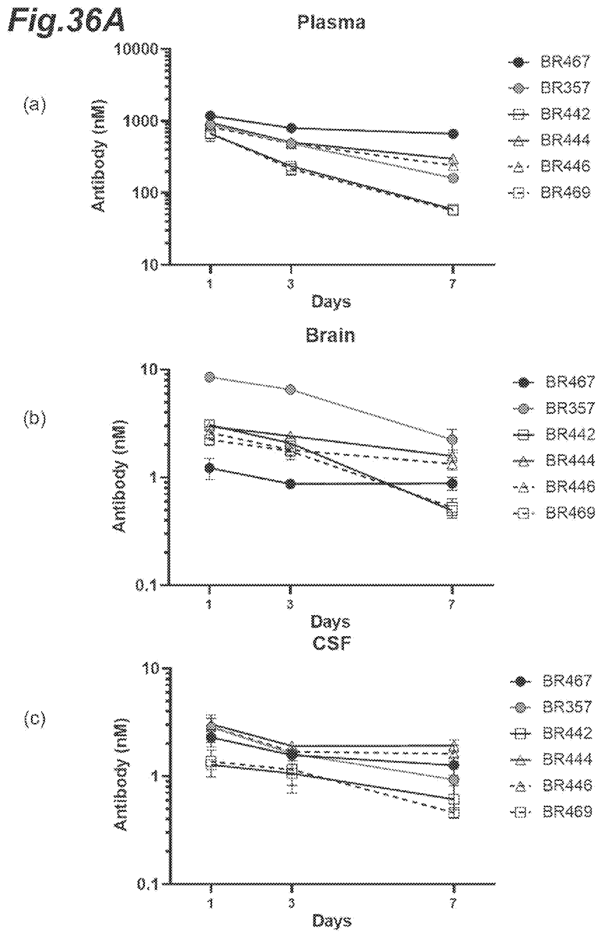

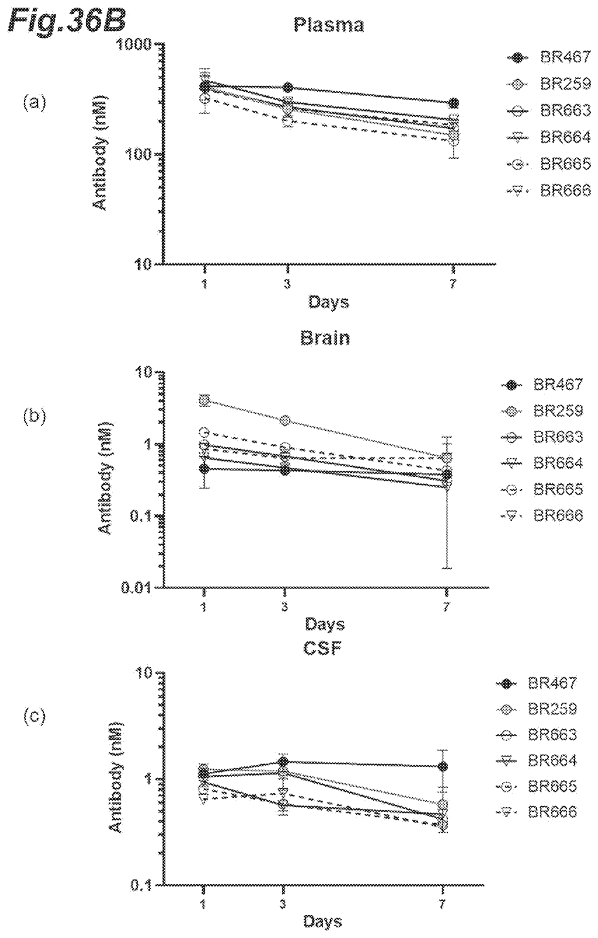

Eleven antibodies of the anti-Aβ antibody BR467 and the bispecific anti-human TfR/Aβ human IgG1 antibodies BR357, BR442, BR444, BR469, BR446, BR259, BR663, BR664, BR665, and BR666 were intravenously administered to male hTfR-ApD-KI (homo) mice at a dose of 20 mg/kg or 10 mg/kg, calculated as an IgG equivalent amount, and at 1, 3, and 7 days post-administration, blood was collected under anesthesia. In addition, blood was collected from Non-Administered to which no antibodies were administered, as a control sample. After collecting CSF, exsanguination via incision of the right atrial appendage and transcardial perfusion with heparin-containing PBS were performed to harvest the brain, which was then divided into the left and right cerebral hemispheres and frozen in liquid nitrogen. In order to measure the concentration of antibody in plasma, the blood collected was centrifuged at 1,200×g for 15 minutes at 4° C., and the supernatant was collected as plasma. After 400 μL of complete Protease Inhibitor Cocktail-containing TBS was added into a collection tube of a BioMasher I (Nippi Inc.), a filter tube was set, the right cerebral hemisphere was placed in it, and a pestle bar was inserted. After centrifugation at 14,000 rpm for 1 minute at 4° C., the filter tube was removed, complete Protease Inhibitor Cocktail-containing TBS was added to bring the total amount including originally added 400 μL to 5 times the weight of the wet tissue, and pipetting was performed 10 times. NP-40 Alternative, 10% solution (Millipore) was added to the final concentration of 1%, and incubated at 4° C. for one hour while rotating. Centrifugation at 15,000 rpm for 1 minute at 4° C. was performed to collect the supernatant as a brain homogenate. The measurement of the antibody concentrations was performed by ELISA as described below. A donkey anti-human IgG-Fcγ chain-specific F(ab′)2 antibody (Jackson ImmunoResearch Inc.) was added to a MaxiSorp Plates (Thermo) and incubated overnight at 4° C. After washing with 0.02% Tween20/PBS three times, blocking was performed at room temperature for one hour by 1% Block Ace (DS Pharma Biomedical Co., Ltd.)/PBS, and then, the brain homogenate, plasma, or CSF diluted using 0.4% Block Ace/PBS were added and incubated at room temperature for 2 hours. After washing with 0.02% Tween20/PBS three times, an HRP-labeled goat anti-human IgG Fcγ chain-specific F(ab′)2 antibody (Jackson ImmunoResearch Inc.) was added and incubated at room temperature for one hour. After washing with 0.02% Tween20/PBS three times, TMB substrate solution (SeraCare) was added and incubated at room temperature for 30 minutes. An equivalent amount of 2N sulfuric acid was added to quench the reaction, and the absorbance at 450 nm and 650 nm was measured with a microplate reader SpectraMax ABS (Molecular Devices, LLC). Calibration curves were created based on the absorbance of standard samples at each concentration, and the antibody concentrations of the respective samples were determined. The results are shown in FIGS. 36A and 36B. In FIG. 36A, compared with BR357, a monovalent bispecific antibody to which one dsscFv was connected, the bivalent-type bispecific antibodies BR442, BR444, BR469, and BR446 to which 2 dsscFvs were connected exhibited approximately ⅓ of the brain antibody concentration at 1 day post-administration. In addition, it was found that blood retention in BR442 and BR469 was lower than BR357. Further, in FIG. 36B, compared with BR259, a monovalent bispecific antibody to which one scFv was connected, the bivalent-type bispecific antibodies BR663, BR664, BR665, and BR666 to which 2 scFvs were connected exhibited approximately less than or equal to ⅓ of the brain antibody concentration at 1 day post-administration.

### Experimental Example 37: Reticulocyte Depletion and Iron Metabolism

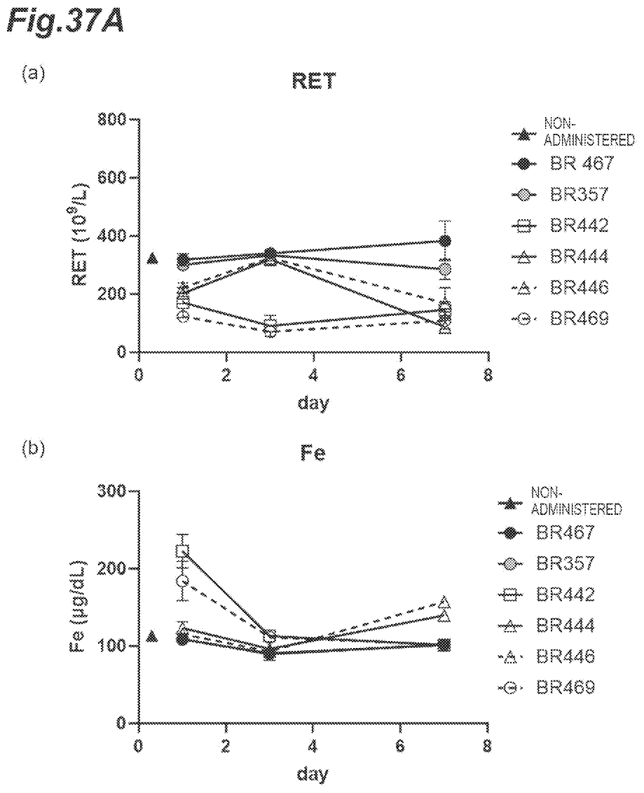

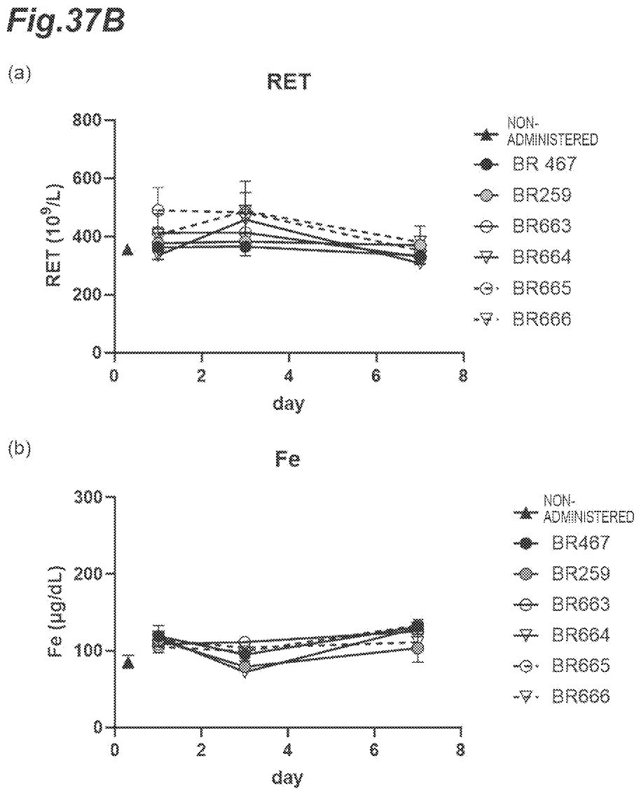

Eleven antibodies, BR467, BR357, BR442, BR444, BR469, BR446, BR259, BR663, BR664, BR665, and BR666 were intravenously administered to male hTfR-ApD-KI mice at dose of 20 mg/kg or 10 mg/kg, calculated as an IgG equivalent amount, and at 1, 3, and 7 days post-administration, blood was collected. Using Capiject II (Terumo), a hematology test was performed. Also, in order to measure the iron, the blood collected was centrifuged at 1,200×g for 15 minutes at 4° C., and the supernatant was collected as plasma. For the iron measurement in a plasma, nitroso-PSAP method was used. The results are shown in FIGS. 37A and 37B. From FIG. 37A, lower values of reticulocyte (RET) in BR442, BR444, BR446, and BR469 were observed, and sometimes associated with an increased iron. The degree of decrease in RET was more marked in BR442 and BR469. In the other antibodies, no decrease in RET or obvious change in iron were observed (FIG. 37B).

### Experimental Example 38: Binding to Amyloid Plaque (APP KI/hTfR-KI Mice)

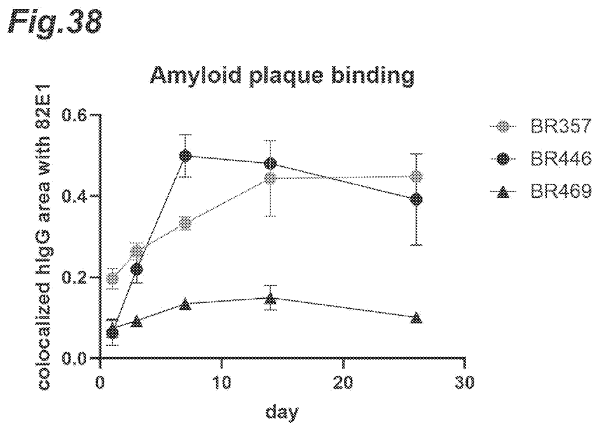

Three antibodies, BR357, BR446, or BR469, were intravenously administered to female APP KI/hTfR-KI mice at a dose of 20 mg/kg, calculated as an IgG equivalent amount, At 1, 3, 7, 14, and 26 days post-administration, blood collection via right auricle incision and transcardial perfusion with heparin-containing PBS were performed to harvest the brain, which was then divided into left and right cerebral hemispheres. The brain was fixed by immersion in 1% paraformaldehyde (TAAB Laboratories Equipment Ltd.) in PBS for 24 hours, follow by cryoprotection through sequential substitution with 10% and then 20% sucrose, and subsequently embedded in Frozen Section Compound (FCS22Clear). Frozen brain sections (10 μm thickness) were prepared using a cryostat CM1860 (Leica) and mounted on a glass slide. After air drying, sections were treated with PBS and 0.1% Triton (trademark) X-100/PBS at room temperature for 5 minutes. After blocking with 0.1% Triton (trademark) X-100 1% normal donkey serum/1× Block Ace at room temperature for one hour, sections were incubated with Alexa555-labeled mouse anti-amyloid antibody (82E1) at room temperature for two hours. After washing with 0.1% Triton (trademark) X-100/PBS, FITC-labeled anti-human IgG antibody (Jackson ImmunoResearch Inc.) was applied and incubated for one hour at room temperature. After washing, mounting was performed with Prolong Glass Antifade Mountant (Thermo). Images were acquired using NanoZoomer digital slide scanner (Hamamatsu Photonics K.K.) and colocalization area of 82E1 and human IgG signals were quantified with NIS Elements software (Nikon Corporation), and the binding to amyloid plaque was quantified (FIG. 38). Binding of BR469 to amyloid plaques was reduced compared with BR357. Binding to amyloid plaque in BR446 was reduced at 1 day post-administration and increased at 7 days, compared to BR357; however, after accounting for time-dependent changes, the binding to amyloid plaques of BR446 was equivalent to that of BR357.

### Experimental Example 39: Construction of Stable Cell Line (SDS-PAGE and Titer)

### (1) Construction of Vector

Similarly to Experimental Example 25, DNA sequences encoding BR441, BR468, BR443, BR445, BR663, and BR664 were inserted into various pPV vectors. Table 20 shows each region constituting each antibody and the combinations of pPV vectors into which the respective regions were inserted. Based on the combinations described in Table 20, pPV vectors containing the respective antibody regions were fused with pPB vectors to construct expression vectors used for establishing stable cell lines of respective antibody.

TABLE 20

| | | | DNA/ | | |
|---|---|---|---|---|---|
| | | Antibody | Amino | pPV | pPB |
| ID | Antibody name | region | acid | vector | vector |
| BR441 | HuAb1-hlgG1- | Heavy chain (H) | DNA | pPV-A | pPB- |
| | N297A- | and scFv region | | | DGV |
| | aT04m02.1 HCbv | Light chain (L) | DNA | pPV-B | |
| | (H-L) | | | | |
| BR441 | HuAb1-hIgG1- | Heavy chain (H) | DNA | pPV-A | pPB- |
| | N297A- | and scFv region | | | DGV |
| | aT04m02.1 HCbv | Light chain (L1, | DNA | pPV-B | |
| | (H-L-L) | L2) | | pPV-C | |
| BR468 | HuAb1-hlgG1- | Heavy chain (H) | DNA | pPV-A | pPB- |
| | N297A- | and scFv region | | | DGV |
| | aT04m0206.1 HCbv | Light chain (L) | DNA | pPV-B | |
| | (H-L) | | | | |
| BR468 | HuAb1-hIgG1- | Heavy chain (H) | DNA | pPV-A | pPB- |
| | N297A- | and scFv region | | | DGV |
| | aT04m0206.1 HCbv | Light chain (L1) | DNA | pPV-B | |
| | (H-L-L) | | | pPV-C | |
| BR443 | HuAb1-hIgG1- | Heavy chain (H) | DNA | pPV-A | pPB- |
| | N297A- | Light chain (L) | DNA | pPV-B | DGV |
| | aT04m02.1 LCbv | and scFv region | | | |
| | (H-L) | | | | |
| BR443 | HuAb1-hIgG1- | Heavy chain (H) | DNA | pPV-A | pPB- |
| | N297A- | Light chain (L1) | DNA | pPV-B | DGV |
| | aT04m02.1 LCbv | and scFv region | | pPV-C | |
| | (H-L-L) | | | | |
| BR445 | HuAb1-hIg1- | Heavy chain (H) | DNA | pPV-A | pPB- |
| | N297A- | Light chain (L) | DNA | pPV-B | DGV |
| | aT04m0206.1 LCbv | and | | | |
| | (H-L) | scFv region | | | |
| BR445 | HuAb1-hIg1- | Heavy chain (H) | DNA | pPV-A | pPB- |
| | N297A- | Light chain (L1) | DNA | pPV-B | DGV |
| | aT04m0206.1 LCbv | and | | pPV-C | |
| | (H-L-L) | scFv region | | | |
| BR663 | HuAb1-hlgG1- | Heavy chain (H) | DNA | pPV-A | pPB- |
| | N297G-aT04m02- | and scFv region | | | DGV |
| | W105S HCbv | Light chain (L) | DNA | pPV-B | |
| | (H-L) | | | | |
| BR664 | HuAb1-hIgG1- | Heavy chain (H) | DNA | pPV-A | pPB- |
| | N297G-aT04m02- | Light chain (L) | DNA | pPV-B | DGV |
| | W105S LCbv | and scFv region | | | |
| | (H-L) | | | | |

### (2) Construction of Stable Expression Cell Line

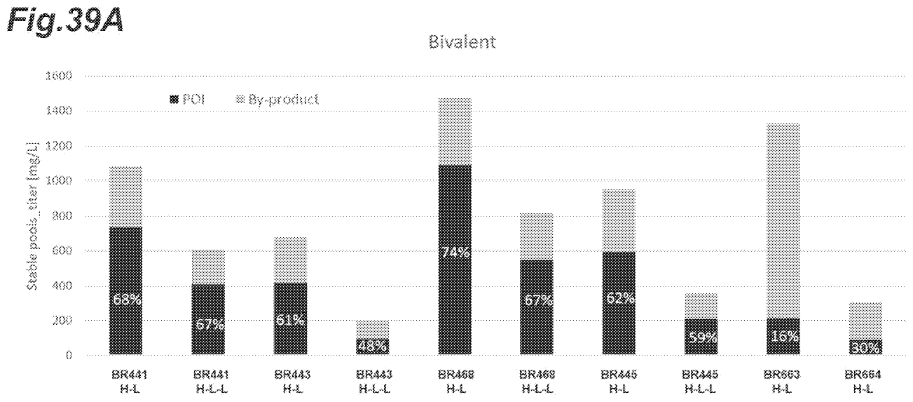

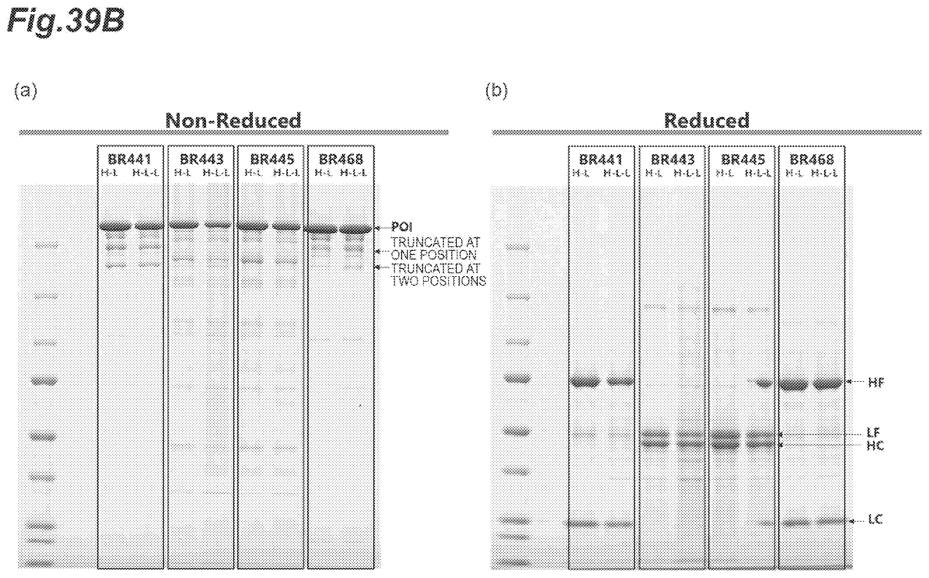

The produced expression vector was transfected into CHO cells by electroporation, and selective culture was performed using a glutamine synthetase inhibitor, MSX (L-Methionine-DL-sulfoximine) in a 50 mL spin tube to establish the stable cell line. The cells were cultured with fed-batch mode for 14 days, and the antibody productivity including by-products was measured using ProA Biosensor for Octet Red96e (Sartorius AG) (FIG. 39A). The culture supernatant was purified by using a MabSelect SuRe (Cytiva, Inc.) column. It was revealed from the results of FIG. 39A that the expression level of the bivalent-type bispecific antibody having two of scFvs or dsscFvs was not markedly improved compared to the monovalent-type bispecific antibody, and in particular, the POI ratio of the bivalent-type bispecific antibodies, BR663 and BR664, in which scFv was connected to the heavy chain C-terminus or the light chain C-terminus, was markedly low.

### (3) SDS-PAGE

The antibodies and SDS sample buffer were mixed at a volume ratio of 1:1, and heated at 70° C. for 10 minutes, and thereby non-reduced samples for SDS-PAGE analysis were prepared. Also, SDS sample buffer containing 10% 3-Mercapto-1, 2-propanediol and the antibodies were mixed at a volume ratio of 1:1, and the mixture was heated at 70° C. for 10 minutes to prepare reduced samples for SDS-PAGE analysis. These non-reduced and reduced samples were applied on the gel, and the electrophoresis was performed by applying current of 20 mA. After that, decolorization was performed with Coomassie staining, and decoloring solution (acetic acid:methanol:distilled water=1:1:8). At the end, the gel was scanned with a densitometer (Bio-Rad Laboratories, Inc., GS-900), and then purity evaluation was performed (FIG. 39B). The POI purity of BR441 and BR468 was 67% to 74%, whereas the POI purity of BR443 and BR445 was 44% to 62%. The POI purity of BR663 was 16%, and truncation of 2 scFvs were observed as the main byproduct. The POI purity of BR664 was 30%, and complex patterns of byproduct were exhibited.

### Experimental Example 40: Construction of Stable Cell Line

### (1) Construction of Vector

A DNA sequence was synthesized in which the variable region of the anti-Aβ antibody DI 8 was applied to the light chain sequence of SEQ ID NO: 119, the first heavy chain sequence of SEQ ID NO: 125, and the second heavy chain and scFv sequence of SEQ ID NO: 127 (BR258) described in Experimental Example 21, the N297G mutation was introduced, and the C-terminal lysine of the first heavy chain was deleted. Accordingly, a vector expressing the first heavy chain gene (DNA sequence: SEQ ID NO: 188; amino acid sequence: SEQ ID NO: 189), the second heavy chain and scFv gene (DNA sequence: SEQ ID NO: 190; amino acid sequence: SEQ ID NO: 191), and the light chain gene (DNA sequence: SEQ ID NO: 196; amino acid sequence: SEQ ID NO: 197) was constructed (BR478). Similarly, a DNA sequence was synthesized in which the variable region of the anti-Aβ antibody DI 8 was applied to the light chain sequence of SEQ ID NO: 119, the first heavy chain sequence of SEQ ID NO: 125, and the second heavy chain and scFv sequence of SEQ ID NO: 183 described in Experimental Example 26, the N297G mutation was introduced, and the C-terminal lysine of the first heavy chain was removed. Accordingly, a vector expressing the first heavy chain gene (DNA sequence: SEQ ID NO: 188; amino acid sequence: SEQ ID NO: 189), the second heavy chain and scFv gene (DNA sequence: SEQ ID NO: 192; amino acid sequence: SEQ ID NO: 193), and the light chain gene (DNA sequence: SEQ ID NO: 196; amino acid sequence: SEQ ID NO: 197) was constructed (BR479). In addition, vectors were constructed in which the variable regions of the first and second heavy chains of BR478 and BR479 were each replaced with the variable region of the anti-Aβ antibody HuAb1. These vectors expressed a new first heavy chain gene (DNA sequence: SEQ ID NO: 247; amino acid sequence: SEQ ID NO: 248, in the constant region) and a new second heavy chain and scFv gene (DNA sequences: SEQ ID NOs: 249 and 251; amino acid sequences: SEQ ID NOs: 250 and 252, in the constant region and scFv region) (BR470 and BR471). The first heavy chain constant regions of BR470, BR471, BR478, and BR479 consist of the amino acid sequence of SEQ ID NO: 240, and the second heavy chain constant region consists of the amino acid sequence of SEQ ID NO: 241. Furthermore, in the vectors expressing the second heavy chain and scFv genes of SEQ ID NOs: 191 and 193, vectors were constructed in which glycine at the ninth residue from the N-terminus of HFR2 (heavy chain framework region 2) and glycine at the third residue from the N-terminus of LFR4 (light chain framework region 4), defined by Kabat numbering, were substituted with cysteine in aT04m02-scFv or aT04m0206-scFv. These vectors expressed new second heavy chain and dsscFv genes (DNA sequences: SEQ ID NOs: 268 and 276; amino acid sequences: SEQ ID NOs: 269 and 277) (BR802 and BR803). The heavy chain variable region of the dsscFv in BR802 consists of the amino acid sequence of SEQ ID NO: 267, and the light chain variable region consists of the amino acid sequence of SEQ ID NO: 265. The heavy chain variable region of the dsscFv in BR803 consists of the amino acid sequence of SEQ ID NO: 275, and the light chain variable region consists of the amino acid sequence of SEQ ID NO: 273. The dsscFv in BR802 (aT04m02-dsscFv) consists of the amino acid sequence of SEQ ID NO: 263. The dsscFv in BR803 (aT04m0206-dsscFv) consists of the amino acid sequence of SEQ ID NO: 271.

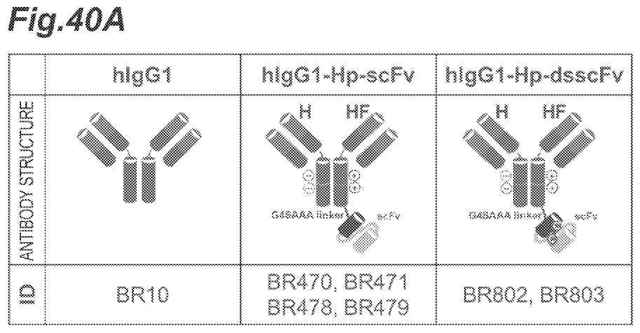

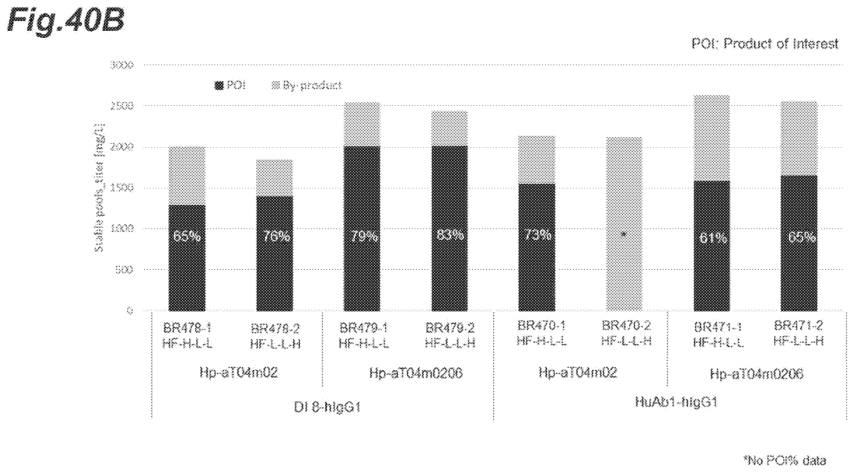

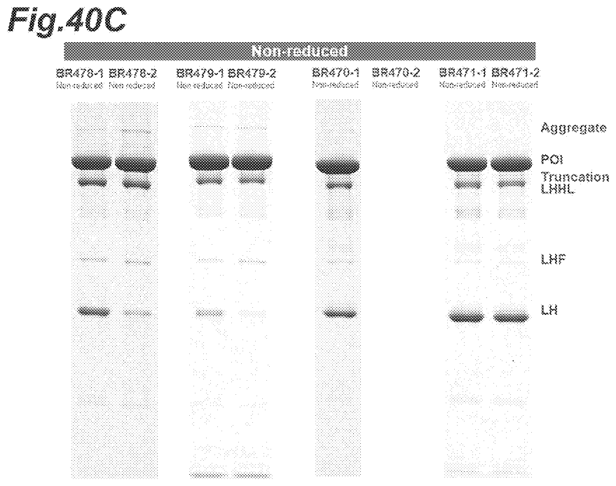

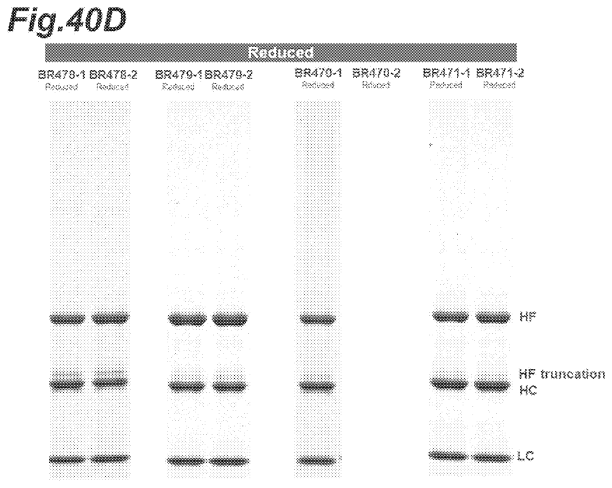

Schematic diagrams of the respective antibodies are shown in FIG. 40A. The combinations of the first and second heavy chains and the light chains included in each of BR478, BR479, BR802, and BR803 are shown in Table 21.

TABLE 21

| | | | DNA/ | |
|---|---|---|---|---|
| | | Antibody | Amino | SEQ ID |
| ID | Antibody name | region | acid | NO: |
| BR478 | DI 8-hlgG1-N297G- | First heavy | DNA | 188 |
| | Hp-HΔK- | chain | Amino | 189 |
| | aT04m02.1 mv | (H) | acid | |
| | H; N297G, K409D, | Second heavy | DNA | 190 |
| | K439E, HΔK | chain | Amino | 191 |
| | HF; N297G, D356K, | (HF) and | acid | |
| | D399K, linker | scFv region | | |
| | (G4SAAA), | Light chain | DNA | 196 |
| | aT04m02-scFv | (L) | Amino | 197 |
| | L; wt | | acid | |
| BR479 | DI 8-hlgG1-N297G- | First heavy | DNA | 188 |
| | Hp-HΔK- | chain | Amino | 189 |
| | aT04m0206.1 mv | (H) | acid | |
| | H; N297G, K409D, | Second heavy | DNA | 192 |
| | K439E, HΔK | chain | Amino | 193 |
| | HF; N297G, D356K, | (HF) and | acid | |
| | D399K, linker | scFv region | | |
| | (G4SAAA), | Light chain | DNA | 196 |
| | aT04m0206- | (L) | Amino | 197 |
| | scFv | | acid | |
| | L; wt | | | |
| BR802 | DI 8-hIgG1-N297G- | First heavy | DNA | 188 |
| | Hp-HΔK- | chain | Amino | 189 |
| | aT04m02.1 dsscFv | (H) | acid | |
| | H; N297G, K409D, | Second heavy | DNA | 268 |
| | K439E, HΔK | chain | Amino | 269 |
| | HF; N297G, D356K, | (HF) and | acid | |
| | D399K, linker | dsscFv region | | |
| | (G4SAAA), | Light chain | DNA | 196 |
| | aT04m02- | (L) | Amino | 197 |
| | dsscFv | | acid | |
| | L; wt | | | |
| BR803 | DI 8-hIgG1-N297G- | First heavy | DNA | 188 |
| | Hp-HΔK- | chain | Amino | 189 |
| | aT04m0206.1_dsscFv | (H) | acid | |
| | H; N297G, K409D, | Second heavy | DNA | 276 |
| | K439E, HΔK | chain | Amino | 277 |
| | HF; N297G, D356K, | (HF) and | acid | |
| | D399K, linker | dsscFv region | | |
| | (G4SAAA), | Light chain | DNA | 196 |
| | aT04m0206- | (L) | Amino | 197 |
| | dsscFv | | acid | |
| | L; wt | | | |

The combinations of the first and second heavy chains and the light chains included in each of BR470, BR471, and BR10 are shown in Table. 22.

TABLE 22

| | | | DNA/ | SEQ |
|---|---|---|---|---|
| | | Antibody | Amino | ID |
| ID | Antibody name | region | acid | NO: |
| BR470 | HuAb1-hIgG1-N297G- | First heavy | DNA | 247 |
| | Hp-HΔK- | chain (H) | Amino | 248 |
| | aT04m02.1_mv | constant region | acid | |
| | H; N297G, K409D, | Second heavy | DNA | 249 |
| | K439E, HΔK | chain (HF) | Amino | 250 |
| | HF; N297G, D356K, | constant region | acid | |
| | D399K, linker | and | | |
| | (G4SAAA), | sc Fv region | | |
| | aT04m02-scFv | Light chain (L) | DNA | 118 |
| | L; wt | constant region | Amino | 119 |
| | | | acid | |
| BR471 | HuAb1-hIgG1-N297G- | First heavy | DNA | 247 |
| | Hp-HΔK- | chain (H) | Amino | 248 |
| | aT04m0206.1_mv | constant region | acid | |
| | H; N297G, K409D, | Second heavy | DNA | 251 |
| | K439Ε, HΔK | chain (HF) | Amino | 252 |
| | HF; N297G, D356K, | constant region | acid | |
| | D399K, linker | and | | |
| | (G4SAAA), | sc Fv region | | |
| | aT04m0206- | Light chain (L) | DNA | 118 |
| | scFv | constant region | Amino | 119 |
| | L; wt | | acid | |
| BR10 | DI 8-hIgG1 | Heavy chain | DNA | 194 |
| | | (H) | Amino | 195 |
| | | | acid | |
| | | Light chain | DNA | 196 |
| | | (L) | Amino | 197 |
| | | | acid | |

Similarly to Experimental Example 25, DNA sequences encoding BR470, BR471, BR478, and BR479 were inserted into various pPV vectors. Table 23 shows each region constituting each antibody and the combinations of pPV vectors into which the respective regions were inserted. Based on the combinations described in Table 23, the pPV vectors containing the respective antibody regions were fused with pPB vectors to construct expression vectors used for establishing stable cell 10 lines for each antibody. Note that the light chain of DI 8-human IgG1 (BR10) consists of the amino acid sequence of SEQ ID NO: 197, and the heavy chain of the same antibody consists of the amino acid sequence of SEQ ID NO: 195. The DNAs encoding these chains consist of the DNA sequences of SEQ ID NOs: 196 and 194, respectively. The DNA sequence encoding the heavy chain of SEQ ID NO: 195 was inserted into a pEE6.4 vector, and the DNA sequence encoding the light chain of SEQ ID NO: 197 was inserted into a pEE12.4 vector. Both vectors were then fused to construct an expression vector used for establishing a stable cell line of BR10.

TABLE 23

| ID | Antibody name | Antibody region | DNA/Amino acid | pPV vector | pPB vector |
|---|---|---|---|---|---|
| BR478-1 | DI 8-hlgG1-N297G- | First heavy | DNA | pPV-B | pPB- |
| | Hp-HΔK- | chain (H) | | | QGV |
| | aT04m02.1 mv | Second heavy | DNA | pPV-A | |
| | (HF-H-L-L) | chain (HF) | | | |
| | | and scFv region | | | |
| | | Light chain (L1) | DNA | pPV-C | |
| | | | | pPV-D | |
| BR478-2 | DI 8-hlgG1-N297G- | First heavy | DNA | pPV-D | pPB- |
| | Hp-HΔK- | chain (H) | | | QGV |
| | aT04m02.1 mv | Second heavy | DNA | pPV-A | |
| | (HF-L-L-H) | chain (HF) | | | |
| | | and scFv region | | | |
| | | Light chain (L1) | DNA | pPV-B | |
| | | | | pPV-C | |
| BR479-1 | DI 8-hlgG1-N297G- | First heavy | DNA | pPV-B | pPB- |
| | Hp-HΔK- | chain (H) | | | QGV |
| | aT04m0206.1 mv | Second heavy | DNA | pPV-A | |
| | (HF-H-L-L) | chain (HF) | | | |
| | | and scFv region | | | |
| | | Light chain (L) | DNA | pPV-C | |
| | | | | pPV-D | |
| BR479-2 | DI 8-hIgG1-N297G- | First heavy | DNA | pPV-D | pPB- |
| | Hp-HΔK- | chain (H) | | | QGV |
| | aT04m0206.1 mv | Second heavy | DNA | pPV-A | |
| | | chain (HF) | | | |
| | | and scFv region | | | |
| | (HF-L-L-H) | Light chain (L) | DNA | pPV-B | |
| | | | | pPV-C | |
| BR470-1 | HuAb1-hIgG1- | First heavy | DNA | pPV-B | pPB- |
| | N297G-Hp-HΔK- | chain (H) | | | QGV |
| | aT04m02.1_mv | Second heavy | DNA | pPV-A | |
| | (HF-H-L-L) | chain (HF) | | | |
| | | and scFv region | | | |
| | | Light chain (L1) | DNA | pPV-C | |
| | | | | pPV-D | |
| BR470-2 | HuAb1-hIgG1- | First heavy | DNA | pPV-D | pPB- |
| | N297G-Hp-HΔK- | chain (H) | | | QGV |
| | aT04m02.1_mv | Second heavy | DNA | pPV-A | |
| | (HF-L-L-H) | chain (HF) | | | |
| | | and scFv region | | | |
| | | Light chain (L1) | DNA | pPV-B | |
| | | | | pPV-C | |
| BR471-1 | HuAb1-hIgG1- | First heavy | DNA | pPV-B | pPB- |
| | N297G-Hp-HΔK- | chain (H) | | | QGV |
| | aT04m0206.1 mv | Second heavy | DNA | pPV-A | |
| | (HF-H-L-L) | chain (HF) | | | |
| | | and scFv region | | | |
| | | Light chain (L) | DNA | pPV-C | |
| | | | | pPV-D | |
| BR471-2 | HuAb1-hIgG1- | First heavy | DNA | pPV-D | pPB- |
| | N297G-Hp-HΔK- | chain (H) | | | QGV |
| | aT04m0206.1_mv | Second heavy | DNA | pPV-A | |
| | (HF-L-L-H) | chain (HF) | | | |
| | | and scFv region | | | |
| | | Light chain (L) | DNA | pPV-B | |
| | | | | pPV-C | |

### (2) Construction of Stable Cell Line

Stable cell lines were established by the method described in Experimental Example 25. The cells were cultured with fed-batch mode for 14 days, and the antibody productivity including by-products was measured using ProA Biosensors for Octet Red96e (Sartorius AG). The culture supernatant was purified using a MabSelect SuRe (Cytiva, Inc.) column. Also, for BR10, transfection into CHO cells was performed by a lipofection method using FreeStyle MAX (Thermo). In a 96-well plate scale, selection culture was performed by MSX (L-methionine-DL-sulfoximine), and then, single cell cloning was performed to obtain a clonal cell line. The antibody productivity in stable cell lines of respective antibodies is shown in FIG. 40B. When comparing BR478 and BR479, it was indicated that the antibody productivity of BR479 having aT04m0206 was higher than that of BR478 having aT04m02. Also, it was found that no marked differences in the antibody productivity and the POI ratio due to the differences in combinations of the first and second heavy chains and the light chains constituting respective antibodies were observed.

### (3) SDS-PAGE

The antibodies and SDS sample buffer were mixed at a volume ratio of 1:1, and heated at 70° C. for 10 minutes, and thereby non-reduced samples for SDS-PAGE analysis were prepared. Also, SDS sample buffer containing 10% 3-Mercapto-1, 2-propanediol and the antibodies were mixed at a volume ratio of 1:1, and the mixture was heated at 70° C. for 10 minutes to prepare reduced samples for SDS-PAGE analysis. These non-reduced and reduced samples were applied on the gel, and the electrophoresis was performed by applying current of 20 mA. After that, decolorization was performed with Coomassie staining, and decoloring solution (acetic acid:methanol:distilled water=1:1:8). At the end, the gel was scanned with a densitometer (Bio-Rad Laboratories, Inc., GS-900), and then purity evaluation was performed (FIGS. 40C and 40D). The POI purity of BR478 was 65% and 76%. The POI purity of BR479 was 79% and 83%. The POI purity of BR470 was 73%. The POI purity of BR471 was 61% and 74%.

### Experimental Example 41: Separation and Purification

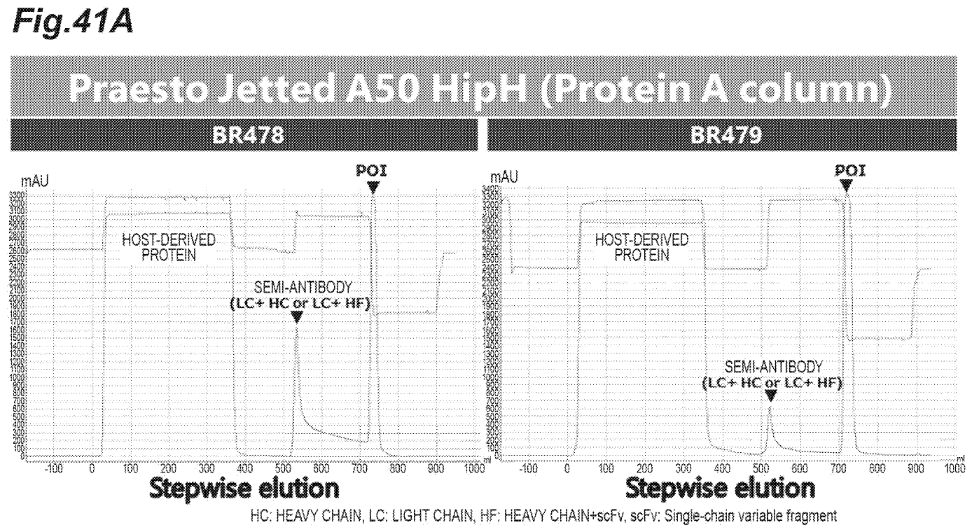

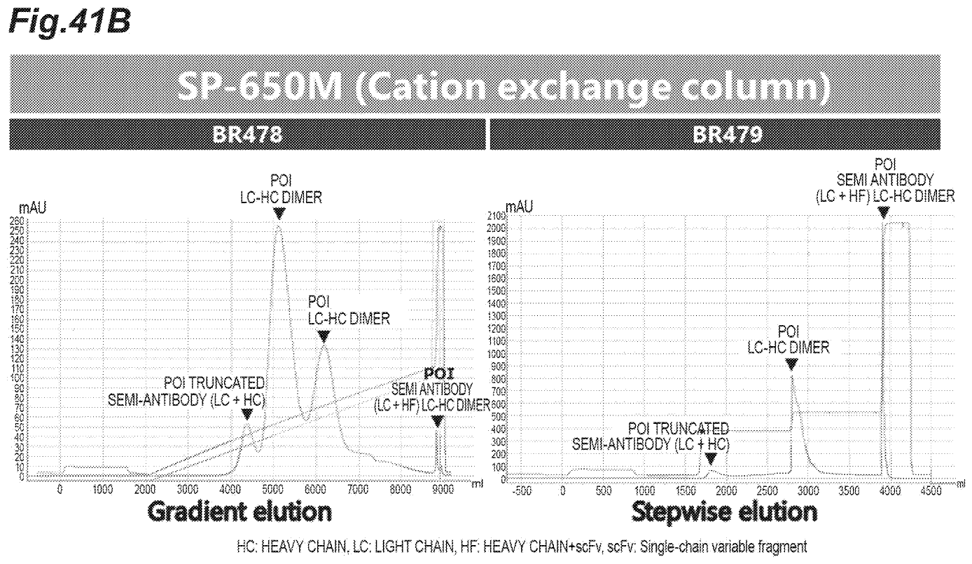

A column, Praesto Jetted A50 HipH (Purolite Corporation), filled with resin was used. The cell culture supernatant of BR478 or BR479 was applied on the Praesto Jetted A50 HipH (Purolite Corporation) column, washed with 50 mM phosphate 50 mM sodium chloride (pH 7.0), and then washed with 100 mM citrate buffer (pH 5.0 or pH 5.5). After that, it was eluted in 100 mM citrate buffer (pH 3.6), and the byproducts and POI were separated (FIG. 41A). Using these column eluates, the separation status was checked by SDS-PAGE. It was revealed from the results of FIG. 41A that it is possible to separate POI and semi-antibodies by a stepwise elution using citrate buffer.

For the separation and purification by cation exchange chromatography, a column filled with resin of 111 mL SP-650M (Tosoh Bioscience, Inc.) was used. Eluates purified by a Praesto Jetted A50 HipH (Purolite Corporation) column were diluted in 25 mM sodium acetate buffer (pH 5.5), applied on the column and washed, and then, the byproducts and POI were separated by gradient elution or a stepwise elution of 25 mM sodium acetate/750 mM sodium chloride buffer (pH 5.5). For the stepwise, 25 mM sodium acetate buffer/112.5 mM sodium chloride buffer (pH 5.5) and 25 mM sodium acetate buffer/165 mM sodium chloride buffer (pH 5.5) were added between 25 mM sodium acetate buffer (pH 5.5) and 25 mM sodium acetate/750 mM sodium chloride buffer (pH 5.5), and thereby a stepwise elution was performed (FIG. 41B). Each fraction was analyzed by SDS-PAGE to confirm the degree of separation. It was revealed from the results of FIG. 41B that it is possible to separate POI, and truncates and semi-antibodies.

### Experimental Example 42: Binding Evaluation to Antigen (Aβ and TfR) (ELISA, SPR, and Flow Cytometry)

### (1) Binding Evaluation to Antigen (Aβ and TfR) (ELISA)

The binding of BR478 and BR479 to protofibril-like Aβ was evaluated according to the ELISA steps described in Experimental Example 18.

The evaluation of the binding specificity of BR478 and BR479 to protofibril-like Aβ was performed by the following competitive ELISA. A monomer Aβ 1-40 solution was added to MaxiSorp Plates and incubated overnight at 4° C. to immobilize the monomer Aβ 1-40 on the plate. After removing the solution, 1% BSA/PBS was added for blocking at room temperature for one hour. Separately, the antibody to be evaluated was mixed with monomer Aβ or protofibril on another plate and incubated at room temperature for one hour with stirring. The blocked plate was then washed three times with 0.02% Tween20/PBS. The stirred mixed solution of the antibody and monomer Aβ or protofibril was added to each well and further incubated at room temperature for one hour. After washing three times with 0.02% Tween20/PBS, the amount of antibody bound to protofibril-like Aβ in a solid phase was detected using an HRP-labeled anti-human IgG antibody (Bethyl Laboratories, Inc.).

The binding evaluation of BR478 and BR479 to human TfR (SEQ ID NO: 243) was performed by ELISA as follows. A His-MBP-human TfR protein was added to MaxiSorp plates and incubated overnight at 4° C. to immobilize the His-MBP-human TfR protein on the plate. After removing the solution, 5% SkimMilk/PBS was added for blocking at room temperature for one hour or more. After washing the plate three times with 0.02% Tween20/PBS, the antibody to be evaluated was added to each well and incubated at room temperature for one hour. After washing three times with 0.02% Tween20/PBS, the amount of antibody bound to human TfR was detected using an HRP-labeled anti-human IgG antibody (Bethyl Laboratories, Inc.).

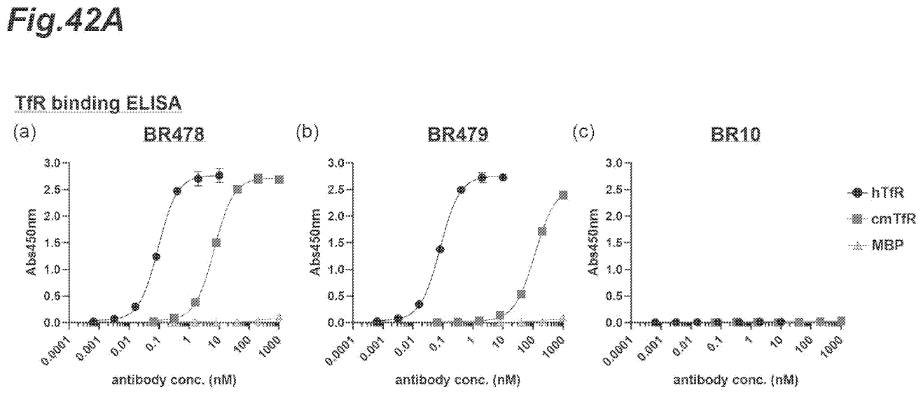

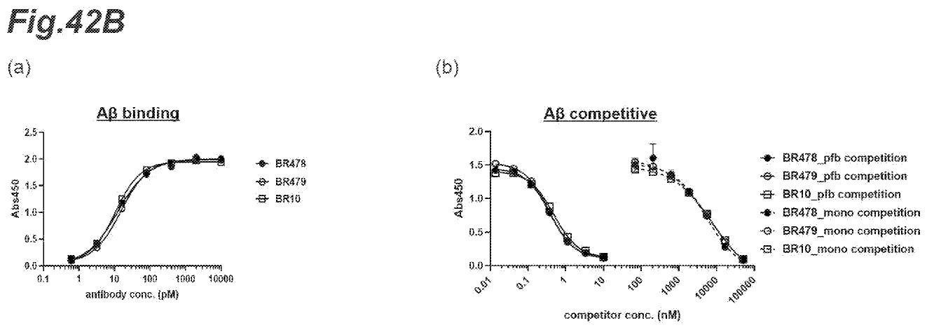

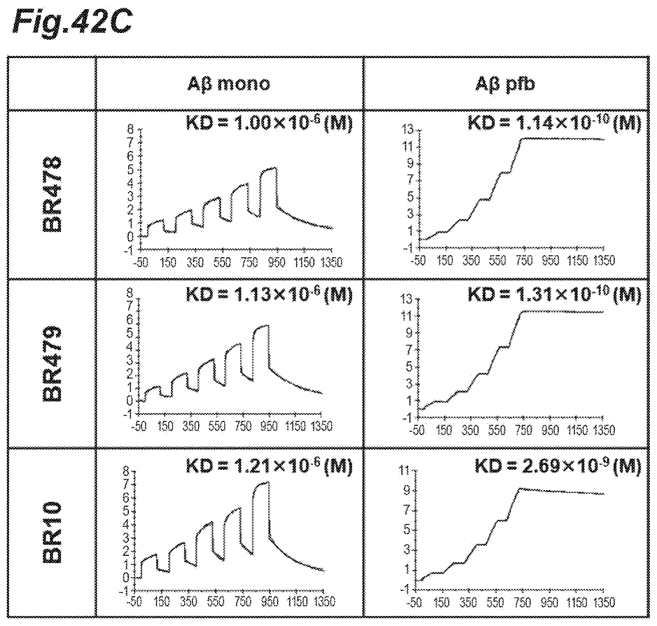

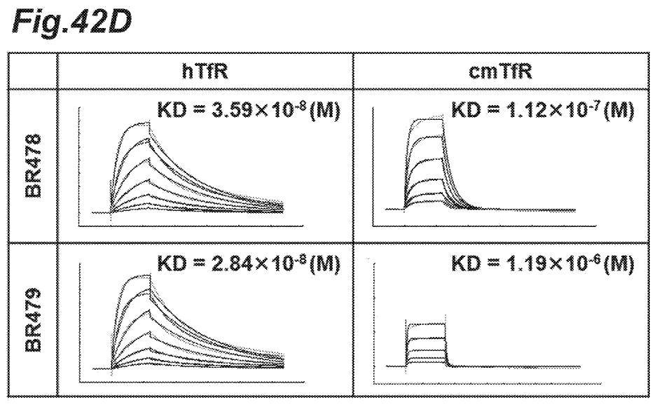

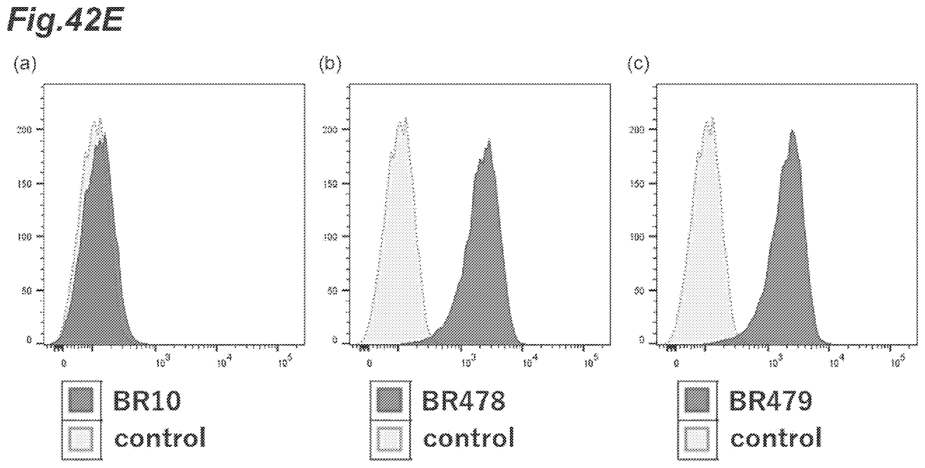

The absorbance obtained by subtracting the background at 650 nm from the absorbance at 450 nm of each antibody concentration is shown in FIG. 42A, and the absorbance at 450 nm of each antibody concentration is shown in FIG. 42B. As shown in FIG. 42A, both BR478 and BR479 exhibited equivalent binding properties to human TfR. On the other hand, it was confirmed that the binding property of BR479 to monkey TfR was lower compared with the binding property to human TfR. Similarly, as shown in FIG. 42B, both BR478 and BR479 exhibited equivalent binding properties to protofibril-like Aβ.

### (2) Aβ SPR

The binding affinity of BR478 and BR479 to monomer Aβ and protofibril was measured by SPR according to the procedure described in Experimental Example 18. The calculated KD values and binding response curves are shown in FIG. 42C. As shown in FIG. 42C, BR478 and BR479 exhibit equivalent binding affinity to monomer Aβ (mono) and protofibril (pfb).

### (3) TfR SPR

The binding affinity of BR478 and BR479 to human TfR (SEQ ID NO: 243) and monkey TfR (SEQ ID NO: 244) was measured using surface plasmon resonance (SPR method, Biacore T200, Cytiva, Inc.) according to the procedure described in Experimental Example 18. The calculated KD values and binding response curves are shown in FIG. 42D below. The binding KD values of BR478 and BR479 to human TfR (hTfR) were 3.59×10⁻⁸ M and 2.84×10⁻⁸ M, respectively, while the binding KD values to monkey TfR (cmTfR) were 1.12×10⁻⁷ M and 1.19×10⁻⁶ M, respectively. These results indicate that BR478 and BR479 exhibited comparable binding affinity to human TfR, whereas BR478 demonstrated approximately ten-fold stronger binding affinity to monkey TfR compared with BR479. It was confirmed that the binding affinity of BR479 to monkey TfR was lower compared with the binding affinity of the antibody to human TfR.

### (4) TfR Flow Cytometry

The binding properties of BR10, BR478, and BR479 to U2OS cells were measured according to the procedure described in Experimental Example 18. Flow cytometry results are shown in FIG. 42E. FIG. 42E indicates that BR478 and BR479 exhibit equivalent binding properties to U2OS cells.

### Experimental Example 43: Fc-Mediated Effector Function (SPR-FcRs, ADCC, C1q Binding, and In Vitro Amyloid Plaque Phagocytosis)

### (1) SPR-FcRs

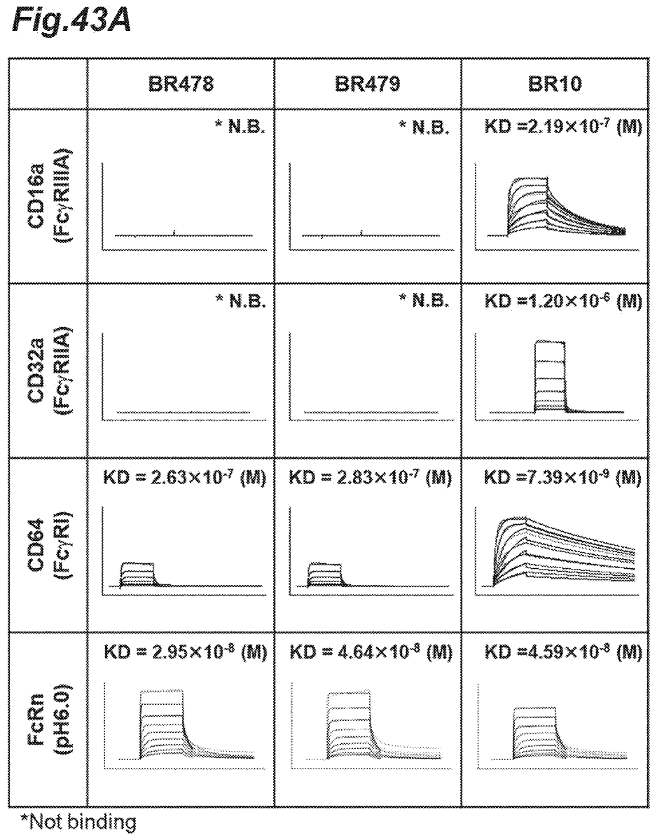

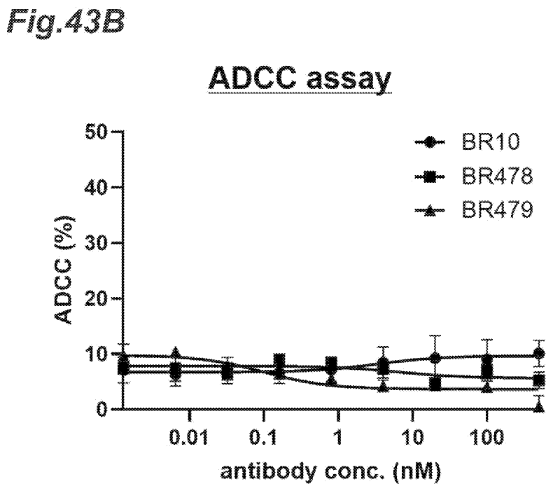

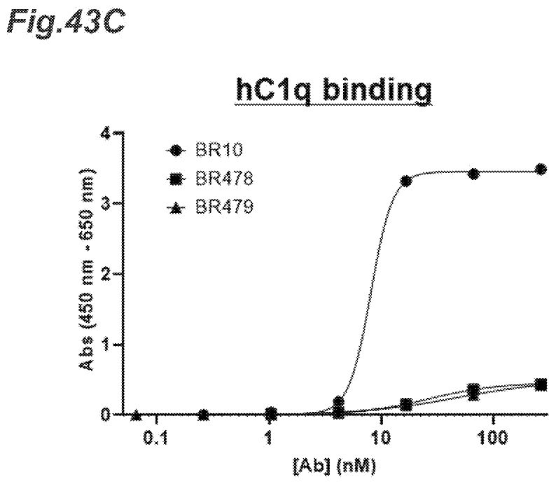

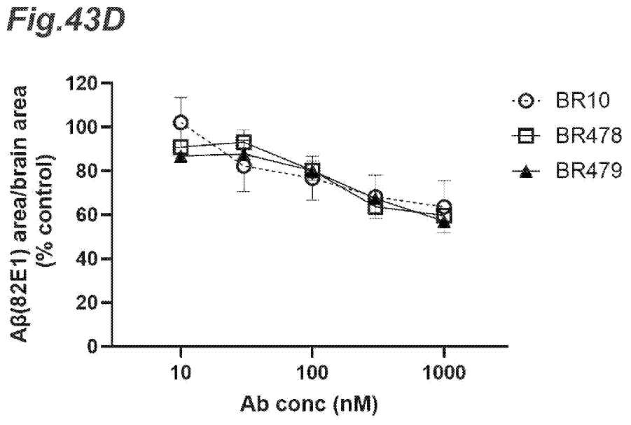

The binding affinity of BR10, BR478, and BR479 to human FcγRI, human FcγRIIA, and human FcγRIIIA was measured using surface plasmon resonance (SPR method, Biacore T200, Cytiva, Inc.) below. The human FcγRI-His, human FcγRIIA-His, or human FcγRIIIA-His (Sino Biological, Inc. for all) that was diluted with HBS-EP+buffer solution having 0.1% BSA was injected and captured on the anti-His antibody which was immobilized on a Series S CM5 sensor chip. Immobilization of the anti-His antibody was performed by a standard amine coupling method according to the vendor's manual. Next, a dilution series of BR10, BR478, and BR479 was sequentially injected, and the binding reactions were observed. Analysis of sensorgram by BIAevaluation Software ver. 2.0 (Cytiva, Inc.) was performed to calculate the binding affinity (KD value) of each antibody by the 1:1 binding model. The binding affinity of BR10, BR478, and BR479 to human FcRn was measured using SPR according to the procedure described in Experimental Example 19. The binding response curves and KD values are shown in FIG. 43A. It was suggested from FIG. 43A that BR478 and BR479 having N297G did not show detectable binding properties to human FcγRIIA and human FcγRIIIA, but the binding properties to human FcγRI are attenuated, and the binding properties to human FcRn are retained, compared to BR10.

### (2) Evaluation of Antibody-Dependent Cellular Cytotoxicity (ADCC) Activity

The lactate dehydrogenase (LDH) activity of BR10, BR478, and BR479 was measured according to the procedure described in Experimental Example 19, and % ADCC is shown in FIG. 43B. FIG. 43B indicates that the ADCC activity of BR478 and BR479 having N297G was markedly lower, similar to BR10.

### (3) C1q Binding

The binding of BR10, BR478, and BR479 to human C1q was measured by ELISA according to the procedure described in Experimental Example 19 (3). The absorbance values at 450 nm, corrected by subtraction of background at 650 nm, are shown in FIG. 43C. As demonstrated in FIG. 43C, BR478 and BR479 having the N297G substitution exhibited markedly reduced binding to human C1q, compared to BR10.

### (4) Evaluation of Fc-Mediated Effector Function (In Vivo Amyloid Plaque Phagocytosis in Brain Tissue of APP KI Mice)

Human monocyte-derived macrophages were prepared as phagocytic cells according to the following procedures. Frozen human peripheral blood mononuclear cells (STEMCELL Technologies) were thawed and added to 10% fetal bovine serum (Gibco)/RPMI 1640 medium (Gibco), and after centrifugation, the cells were suspended in 0.5% BSA/2 mM EDTA/PBS. Classical monocytes were isolated using the Classical Monocyte Isolation Kit human (Miltenyi Biotec) according to the kit's instructions. The isolated cells were suspended in culture medium (10% fetal bovine serum, 2 mM L-Glutamine (Gibco)/Penicillin-Streptomycin (FUJIFILM Wako Pure)/RPMI 1640), and 50 ng/mL GM-CSF (R&D Systems) and 25 ng/ml M-CSF (R&D Systems) at final concentrations, were added. 2×10⁶ cells were seeded into each well of a 6-well culture plate, and culture was initiated at 37° C. and 5% CO₂. On Day 3 of culture, an equal volume of culture medium containing 50 ng/mL GM-CSF and 25 ng/ml M-CSF was added. When the cells reached confluence by Day 6 of culture, they were detached using Accutase (Nacalai Tesque) and passaged. On Day 7 of culture, the cells were detached using Accutase, centrifuged, and then suspended in culture medium containing 50 ng/mL GM-CSF and 25 ng/mL M-CSF to a concentration of 1-1.2×10⁵ cells/mL, and subjected to a phagocytosis assay. As targets of phagocytosis, 10-month-old female APP KI mice were used. Exsanguination by right auricle incision and transcardial perfusion with heparin-containing PBS were performed, and the brain was collected. The brain was cut in half, fixed at 4° C. for 24 hours in 4% paraformaldehyde phosphate buffer solution (FUJIFILM Wako Pure), then washed with PBS, and frozen in liquid nitrogen. Brain sections at a thickness of 20 μm were prepared using a CM1860 cryostat (Leica), mounted on poly-D-lysine-coated 12 mm coverslips (neu Vitro Corporation), placed in a 24-well plate, air dried at 45° C., and washed with PBS. An antigen retrieval solution prepared by diluting Antigen Retrieval Buffer (100× Tris-EDTA Buffer pH 9.0, abcam) 100 times with distilled water was added, and incubated at 60° C. for 10 hours, followed by washing three times with PBS. Sections were incubated at room temperature for one hour with 10, 30, 100, 300, 1000 nM of BR10, BR478 or BR479, or 1000 nM of control antibody and then washed three times with PBS. Human monocyte-derived macrophages were added at 1-1.2×10⁵ cells/well, and cultured at 37° C. in 5% CO₂ for 2 days. After culture, the samples were fixed at room temperature for 30 minutes with 2% paraformaldehyde solution. After washing four times with PBS, blocking was performed at room temperature for 30 minutes with 5% Block Ace (DS Pharma Biomedical Co., Ltd.)/0.1% Tween20/PBS. Anti-Human Amyloid beta (N) (82E1) Mouse IgG MoAb (IBL Co., Ltd.), fluorescently labeled using Alexa Fluor 555 Antibody Labeling Kit (Molecular probes) was diluted with 1% Block Ace/0.1% Tween20/PBS, mounted on coverslips, and incubated at room temperature for one hour. After washing three times with PBS, the samples were mounted using Prolong Gold Antifade Mountant (Molecular probes), imaged with NanoZoomer (Hamamatsu Photonics), and the signal of the 82E1 antibody was calculated as the amyloid plaque area using NIS Elements (Nikon). (FIG. 43D). The mean values and data ranges of two independent experiments are shown. Sections treated with BR10, BR478 and BR479 showed a similar concentration-dependent reduction in anti-Aβ antibody-positive signals.

### Experimental Example 44: Immunogenicity (Epivax)

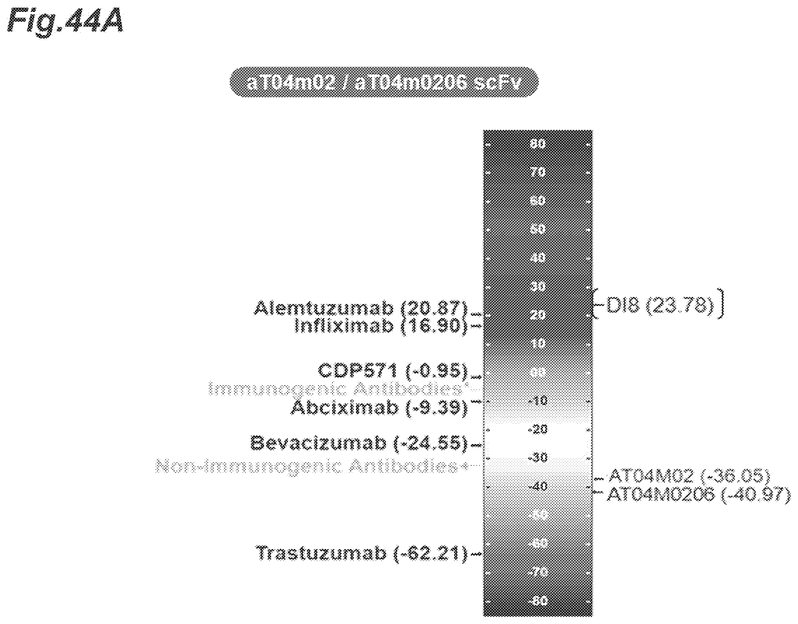

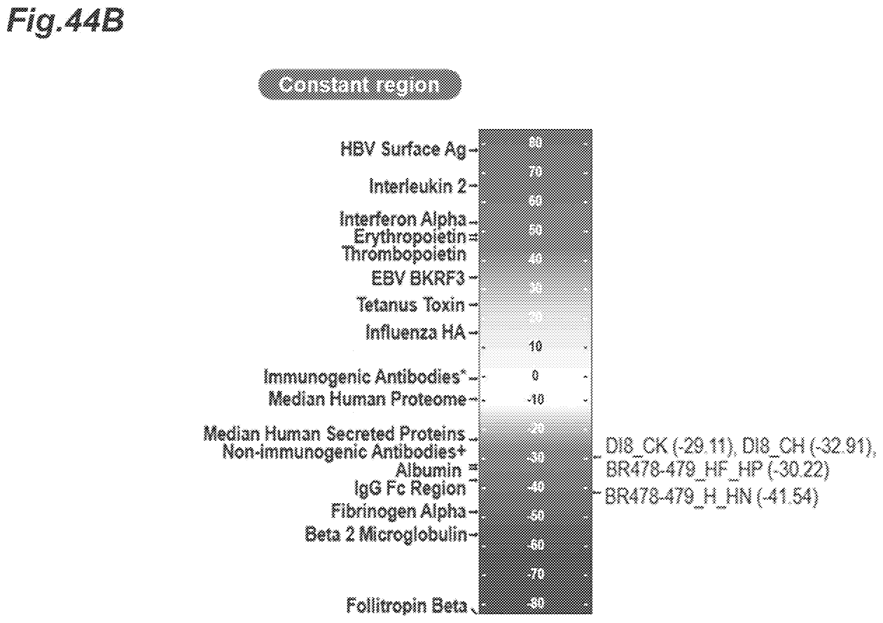

The immunogenicity of the sequence was evaluated in silico using mathematical algorithms. Specifically, the EpiMatrix (registered trademark) Treg-adjusted Score was determined as an index of the immunogenicity for the variable and constant regions of the anti-TfR antibody or anti-Aβ antibody of BR478 and BR479. A lower score indicates a lower likelihood that the sequence is immunogenic, and reference thresholds have been established based on scores of antibodies known to have low immunogenicity or antibodies confirmed to be immunogenic. The immunogenicity scores for each region of BR478 and BR479 are shown in FIGS. 44A and 44B, and Table 24. As a result, it was predicted that aT04m02-scFv and aT04m0206-scFv (the variable regions (scFv) in Table 24), as well as the constant regions of each heavy chain and light chain, are sequences with a low immunogenic risk.

TABLE 24

| | | | Tregitope-Adjusted | | |
|---|---|---|---|---|---|
| [Variable | | | EpiMatrix Protein Score | | |
| region] | | | | | Full length |
| Anti- | | | | | (Heavy |
| body | | SEQ | Heavy | Light | chain + light |
| region | ID | ID NO: | chain | chain | chain) |
| Variable | BR478, | 238 and | 31.32 | 15.36 | 23.78 |
| region | BR479 | 239 | | | |
| (IgG) | and | | | | |
| | BR10 | | | | |
| Variable | BR478 | 66 and 65 | −38.85 | −32.95 | −36.05 |
| region | BR479 | 72 and 65 | −48.19 | −32.95 | −40.97 |
| (scFv) | | | | | |
| [Constant | | | Tregitope-Adjusted | | |
| region] | | | EpiMatrix Protein Score | | |
| Anti- | | | First | Second | |
| body | | SEQ ID | heavy | heavy | Light |
| region | ID | NO: | chain | chain | chain |
| Constant | BR478 | 240, 241 | −41.54 | -30.22 | −29.11 |
| region | and | and 119 | | | |
| | BR479 | | | | |
| | BR10 | 283 and | −32.91 | −32.91 | −29.11 |
| | | 284 | | | |

### Experimental Example 45: Off-Target Assay (Retrogenix)

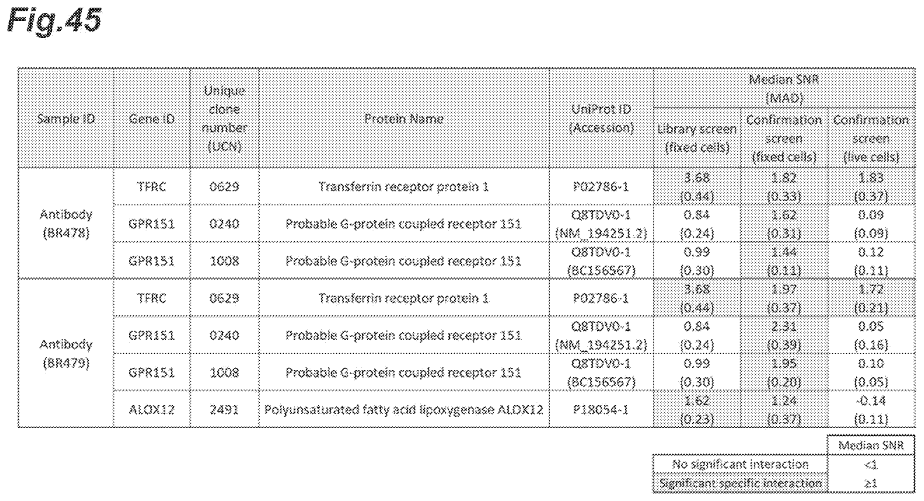

Off target screening to more than 6500 human proteins (membrane proteins, secretory proteins, and heterodimers) expressing on human embryonic kidney (HEK) 293 cell surfaces was performed on BR478 and BR479, using technology of Charles River Laboratories, Inc. (Retrogenix [registered trademark] Cell Microarray Technology, an evaluation system using a huge number of expression vectors encoding human proteins). As a result, binding to TfR and GPR151 (BR478 and BR479), as well as to ALOX12 (BR479 only) were observed in the fixed cell condition of HEK293 cells at the evaluated concentration (0.2 μg/mL); however, non-specific off-target bindings were not observed for both BR478 and BR479 in the live cell condition, and only the binding to the target TfR, was exhibited (FIG. 45). The difference in the results between the two conditions was considered to be caused by increased permeability of cell membranes due to the fixing procedure in the fixed cell, raising the sensitivity of fixed cells generally to be higher than live cells.

### Experimental Example 46: Mouse PK (hTfR-ApD-KI Mouse)

(1) Sample Preparation from Animals Given Bispecific Anti-Human TfR/Aβ Human IgG1 Antibody

Three antibodies of the anti-Aβ human IgG1 antibody BR10 and the bispecific anti-human TfR/Aβ human IgG1 antibodies BR478 and BR479 were each intravenously administered to male hTfR-ApD-KI (homo) mice at a dose of 10 mg/kg, calculated as an IgG equivalent amount. At 5 minutes, 6 hours, 1, 3, 7, 14, and 28 days post-administration, blood was collected under anesthesia. After collecting CSF, exsanguination via incision of the right atrial appendage and transcardial perfusion with heparin-containing PBS were performed to harvest the brain, which was then divided into the left and right cerebral hemispheres and were frozen in liquid nitrogen. The blood was centrifuged at 1,200×g for 15 minutes at 4° C., and the supernatant was collected as plasma. In order to measure the antibody concentration in the brain, 400 μL of complete Protease Inhibitor Cocktail-containing TBS was added into a collection tube of a BioMasher I (Nippi Inc.), and then, a filter tube was set, the right cerebral hemisphere was placed in it, and a pestle bar was inserted. After centrifugation at 14,000 rpm for 1 minute at 4° C., the filter tube was removed, complete Protease Inhibitor Cocktail-containing TBS was added to bring the total amount including originally added 400 μL to 5 times the weight of the wet tissue, and pipetting was performed 10 times. NP-40 Alternative, 10% solution (Millipore) was added to the final concentration of 1%, and incubated at 4° C. for one hour while rotating. Centrifugation at 15,000 rpm for 1 minute at 4° C. was performed to collect the supernatant as a brain homogenate.

### (2) Measurement of Antibody Concentration

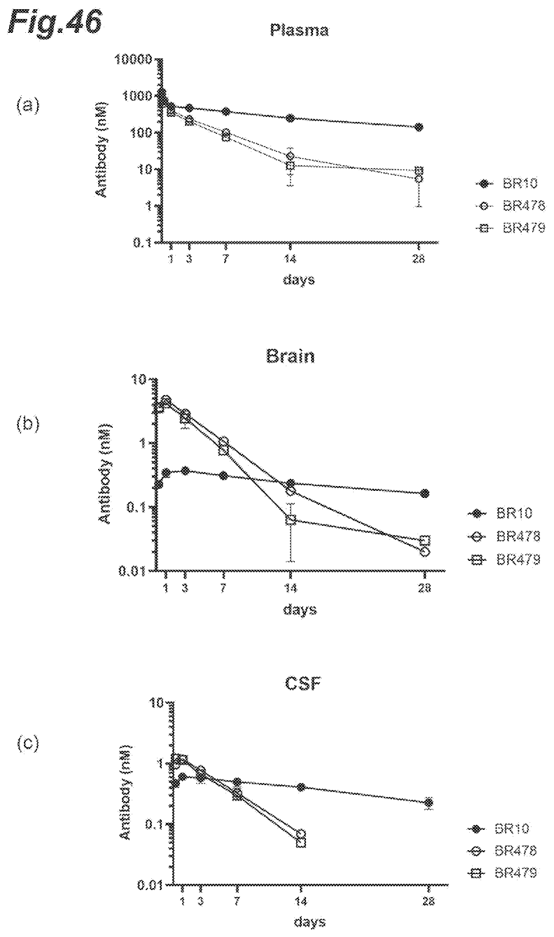

The measurement of the antibody concentrations was performed by ELISA as described below. A donkey anti-human IgG-Fcγ chain-specific F(ab′)2 antibody (Jackson ImmunoResearch Inc.) was added to MaxiSorp Plates (Thermo) and incubated overnight at 4° C. After washing with 0.02% Tween20/PBS three times, blocking was performed at room temperature for one hour by 1% Block Ace (DS Pharma Biomedical Co., Ltd.)/PBS, and then, the brain homogenate, plasma, or CSF diluted using 0.4% Block Ace/PBS were added and incubated at room temperature for 2 hours. After washing with 0.02% Tween20/PBS three times, an HRP-labeled goat anti-human IgG Fcγ chain-specific F(ab′)2 antibody (Jackson ImmunoResearch Inc.) was added and incubated at room temperature for one hour. After washing with 0.02% Tween20/PBS three times, TMB substrate solution (SeraCare) was added and incubated at room temperature for 30 minutes. An equivalent amount of 2N sulfuric acid was added to quench the reaction, and the absorbance at 450 nm and 650 nm was measured with a microplate reader SpectraMax ABS (Molecular Devices, LLC). Calibration curves were created based on the absorbance of standard samples at each concentration, and the antibody concentrations of the respective samples were determined (FIG. 46). It was indicated that the antibody concentrations in the brain at 1 day post-administration were remarkably higher in the bispecific antibodies BR478 and BR479 than BR10. For the antibody concentrations in the brain, plasma, and CSF at 1, 3, and 7 days post-administration, there were no marked differences observed between the bispecific antibodies BR478 and BR479.

### Experimental Example 47: Reticulocyte Deletion (hTfR-ApD-KI Mouse)

(1) Sample Preparation from Animals Given Bispecific Anti-Human TfR/Aβ Human IgG1 Antibody

Two antibodies of the bispecific anti-human TfR/Aβ human IgG1 antibodies BR478 and BR479 were intravenously administered to male hTfR-ApD-KI mice at a dose of 10 mg/kg, calculated as an IgG equivalent amount, and the blood was collected at 6 hours, 1, 3, 7, 14, and 28 days post-administration.

### (2) Blood Test

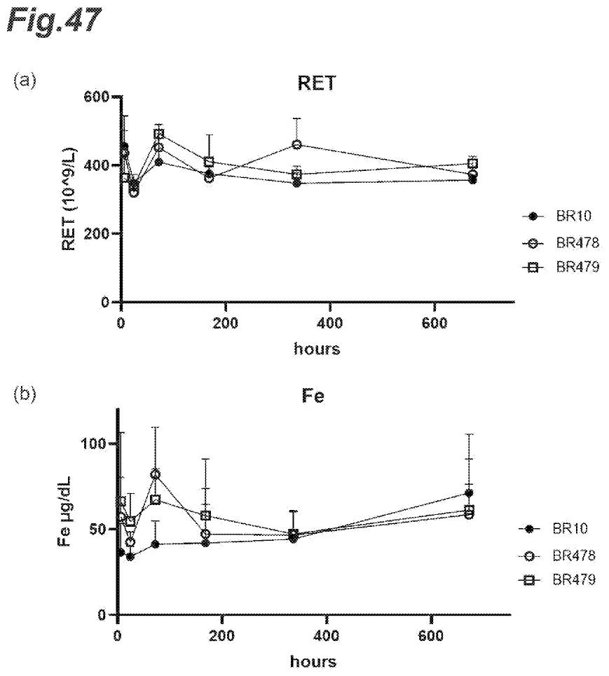

Hematology test and blood chemistry test were performed using the blood samples obtained by using Capiject II (Terumo Corporation) in the above (1). Also, nitroso-PSAP method was used for the iron measurement in the plasma. As a result, there were no toxicologically meaningful changes in reticulocyte (RET) and iron for both antibodies BR478 and BR479 (FIG. 47).

### Experimental Example 48: Effect of Single Administration on Oligomer Aβ in Brain Tissue (APP/hTfR-ApD-KI Mice)

### (1) Establishment of APP-KI/hTfR-ApD-KI Mice

APP KI mice in which amyloid plaque is formed in the brain (RBRC06344, Institute of Physical and Chemical Research) were intercrossed with hTfR-ApD-KI mice (Experimental Example 16). APP KI/hTfR-ApD-KI (hetero/hetero) mice obtained by crossing were further crossed with siblings to produce APP KI/hTfR-ApD-KI (homo/homo) mice. After generation, the strain was maintained by sibling reproduction.

(2) Sample Preparation from Animals Given Bispecific Anti-Human TfR/Aβ Human IgG1 Antibody

6-month-old male APP KI/hTfR-ApD-KI mice were administrated the anti-Aβ human IgG1 antibody BR10 at a dose of 10 mg/kg and the bispecific anti-human TfR/Aβ human IgG1 antibodies BR478 and BR479 at doses of 10 mg/kg and 30 mg/kg, calculated as an IgG equivalent amount by intravenous injection via the tail vein (10 mL/kg). In the control group, PBS was administered intraperitoneally. One week after the final administration, blood was collected under anesthesia, followed by exsanguination via right auricular incision and transcardial perfusion with PBS containing heparin. The brain was harvested and sectioned into left and right cerebral hemispheres. The right cerebral hemisphere was weighed to determine wet tissue weight, frozen in liquid nitrogen, and stored at −80° C. Ten times the amount of the wet tissue weight of TBS was added and tissue was homogenized using a homogenizer (BRANSON, Inc.), and then, centrifugation was performed at 16,000×g for 60 minutes at 4° C., and the supernatant was collected as TBS soluble fraction (16k_sup). The obtained supernatant was further centrifuged at 100,000×g for 60 minutes at 4° C., and the supernatant was collected as the TBS-soluble fraction (16k/100k_sup). The collected blood was centrifuged at 1,200×g for 15 minutes at 4° C., and the supernatant was collected as plasma.

### (3) Measurement of Oligomer Aβ Concentrations in TBS-Soluble Fraction

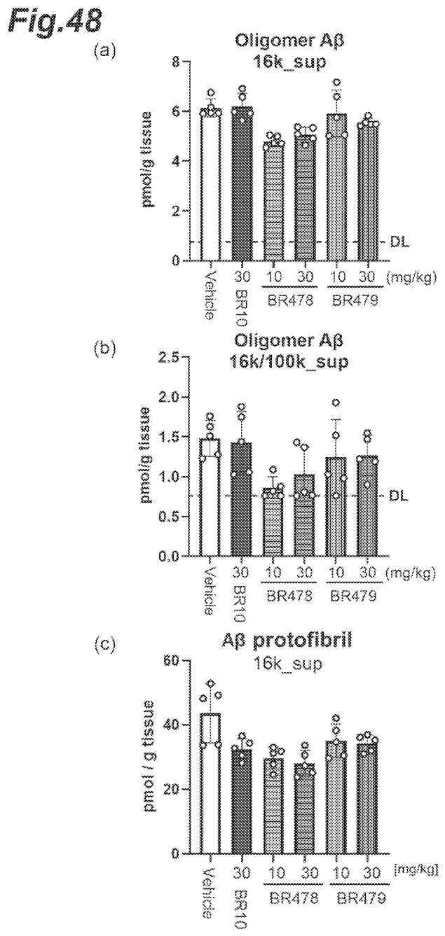

For the TBS-soluble fraction, the reaction was performed using Human Amyloidβ Oligomers (82E1-specific) Assay Kit (IBL Co., Ltd.) according to the instructions in the kit. After the reaction, the absorbance at 450 nm was measured with a microplate reader SpectraMax (Molecular Devices, LLC). The concentrations of oligomer Aβ included in the TBS soluble fraction were determined, and further, the amount of oligomer Aβ per g of wet brain tissue was calculated (FIG. 48). In FIG. 48 (a) and (b), the amount of oligomer Aβ of 0.76 μmol/g tissue was indicated as the detection limit (DL), and individuals of less than the detection limit were shown as 0.76 μmol/g tissue. Both in 16k_sup and 16k/100k_sup, decreased oligomer Aβ was not observed in the group administered BR10 at 30 mg/kg; however, decreased oligomer Aβ was observed in the group administered BR478 at 10 mg/kg or 30 mg/kg, compared to the control group. In addition, a decreasing tendency of oligomer Aβ was also observed in the group administered BR479 at 30 mg/kg.

### (4) Measurement of Aβ Protofibril Concentrations in TBS-Soluble Fraction

The Aβ protofibril-capturing antibody BR10 was labeled onto magnetic beads to prepare antibody-labeled magnetic beads. The prepared antibody-labeled magnetic beads were diluted with Discovery/Assay Buffer (EMD Millipore) to a concentration of 100 μg/mL. Fifty microliters of the antibody-labeled bead solution and 150 μL of TBS-soluble fraction 16k_sup diluted with Discovery Standard Diluent (EMD Millipore) were added to each well of a 96-well V-bottom plate (Axygen), and the plate was shaken at 27° C. and 800 rpm for 2 hours. After washing the 96-well V-bottom plate with washing buffer, a fluorescent dye-labeled anti-Aβ antibody, mouse 3D6 antibody (ADx Neurosciences), was added to each well, followed by shaking at 27° C. and 800 rpm for 1 hour. After washing the 96-well V-bottom plate with washing buffer, 12 μL of Elution Buffer B (EMD Millipore) was added to each well, and the plate was shaken at 27° C. and 800 rpm for 10 minutes. After shaking, 10 μL was collected from each well of the plate and added to each well of a 384-well plate (Aurora Microplates) to which 10 μL of Buffer D (EMD Millipore) had been added in advance. The 384-well plate was shaken at 25° C. and 1000 rpm for 2 minutes, and fluorescence derived from the fluorescently labeled antibody was counted using SMCxPRO (EMD Millipore), and the Aβ protofibril concentration was calculated (FIG. 48 (c)). The quantification results were normalized to wet brain weight. Compared with the control group, a similar tendency toward reduction in Aβ protofibrils was observed in the group administered 30 mg/kg of BR10, the groups administered 10 mg/kg or 30 mg/kg of BR478, and the groups administered 10 mg/kg or 30 mg/kg of BR479.

### Experimental Example 49: Monkey PK

(1) Sample Preparation from Animals Given Bispecific Anti-Human TfR/Aβ Antibody

Two antibodies, BR478 and BR479 were each intravenously administered to male cynomolgus monkeys at a dose of 5 and 30 mg/kg, and the peripheral blood was collected before administration, and at 5 minutes, 2, 6, 24, 72, 168, 336, 504, and 672 hours post-administration to separate the serum. Also, CSF was collected at 24, 72, 168, 336, 504, and 672 hours post-administration, and, along with the serum, the measurement of concentrations of humanized anti-hTfR antibodies in (2) below was performed.

### (2) Measurement of Concentration of Bispecific Anti-Human TfR/Aβ Antibody in Serum and CSF

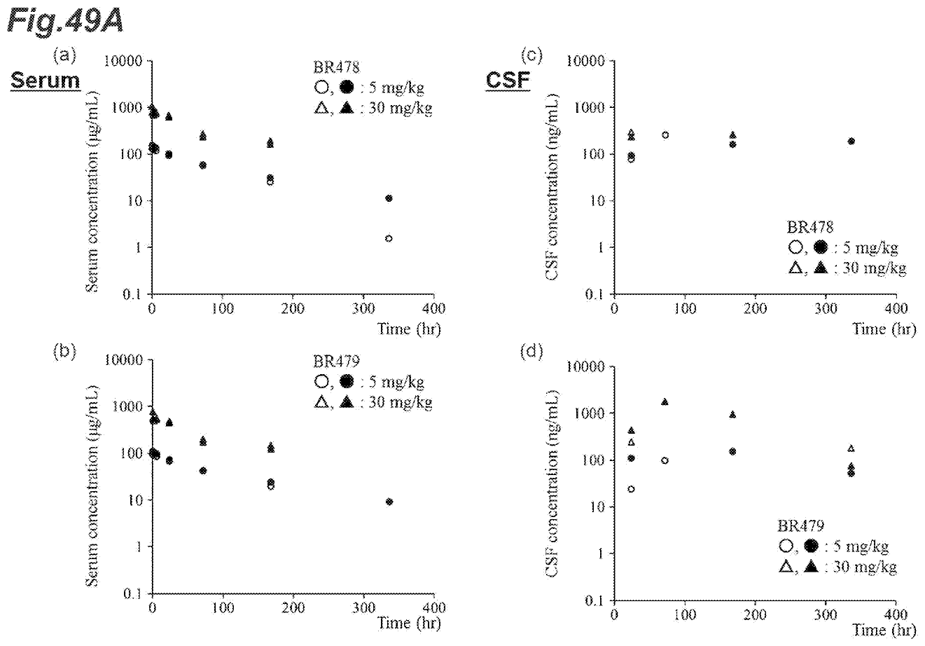

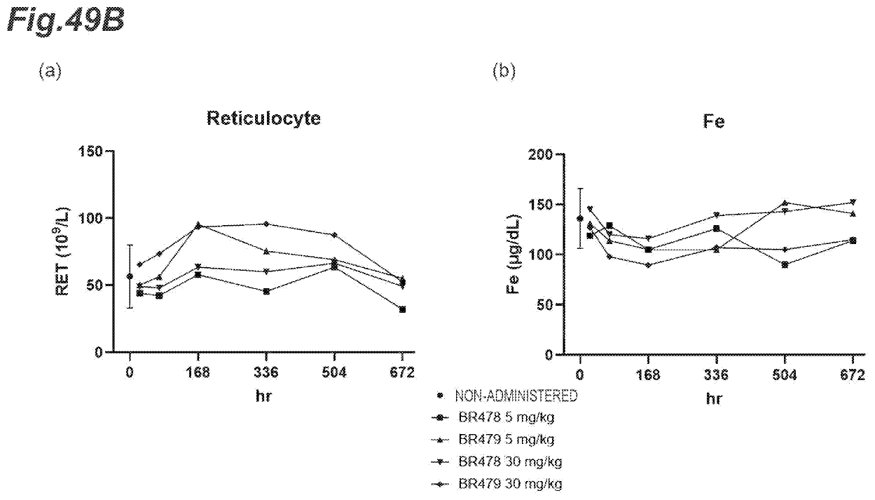

To measure the concentrations of the administered antibodies in cynomolgus monkey serum and CSF, diluted serum and CSF samples were added to the plate in which monomer Aβ 1-40 (Anaspec, Inc.) was immobilized, an HRP-labeled anti-human IgG antibody was added and incubated, and then, TMB substrate solution was added and incubated, and sulfuric acid was added to quench the reaction. By measuring the absorbance at 450 nm and 650 nm of the liquids on the plate and the generating calibration curves based on the absorbance of standard samples at each concentration, the antibody concentrations of the respective samples were determined. By the assay, calibration curves in the range of 1.25 to 500 μg/ml and 2.5 to 500 ng/ml for the serum and CSF were obtained, respectively. The results less than the detection limit were reported as BQL. FIG. 49A shows pharmacokinetic profiles of BR478 and BR479. For the transition of concentrations in serum after the administration, there were no marked differences observed between the bispecific antibodies BR478 and BR479. For the concentrations in CSF, there were no marked differences observed between BR478 and BR479 at 5 mg/kg, whereas BR479 exhibited high values at 30 mg/kg. Symbols (white circle, black circle, white triangle, and black triangle) in the figure indicate the concentration profile for each individual.

### (3) Blood Test

Hematology test and blood chemistry test were performed using the blood samples of before administration, and at 24, 72, 168, 336, 504, and 672 hours post-administration in (1) above. Also, nitroso-PSAP method was used for the iron measurement in serum. As a result, there were no apparent changes attributable to BR478 and BR479 in reticulocyte (RET) and iron (FIG. 49B).

### Experimental Example 50: Binding to Amyloid Plaques (APP KI/hTfR-ApD-KI Mice)

(1) Sample Preparation from Animals Given Bispecific Anti-Human TfR/Aβ Human IgG1 Antibody

To 5-month-old male APP KI/hTfR-ApD-KI (homo/homo) mice, the anti-Aβ human IgG1 antibody BR10 at a dose of 10 mg/kg, and the bispecific anti-human TfR/Aβ human IgG1 antibodies BR478 and BR479 at doses of 10 mg/kg, calculated as an IgG equivalent amount were administered via the tail vein (10 mL/kg). At 3, 7, and 14 days post-administration, exsanguination via right auricular incision followed by transcardial perfusion with PBS containing heparin was performed, and transcardial perfusion with 4% paraformaldehyde/phosphate buffer solution (PB) was performed in a similar route to harvest the brain. The collected brain was placed in 4% paraformaldehyde/PB, and after immersed and fixed at 4° C. for 24 hours, transferred to PBS and immersed therein.

### (2) Tissue Clearing and Whole-Mount-IHC

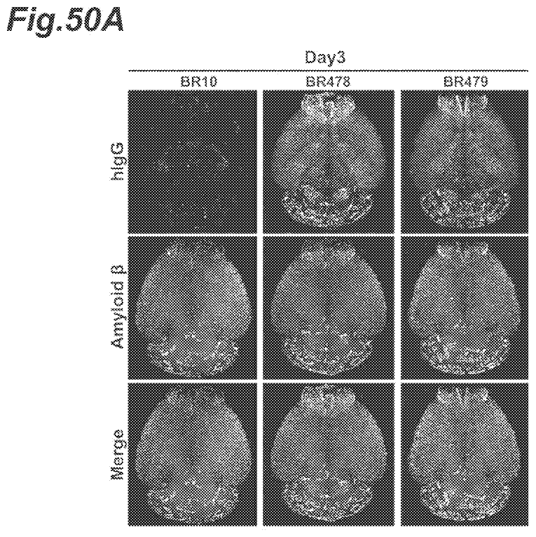

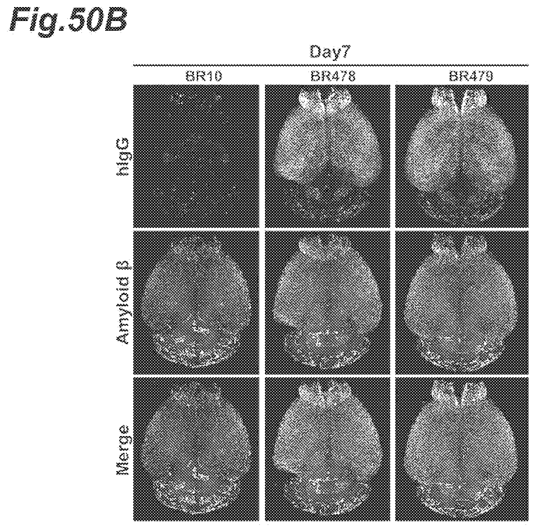

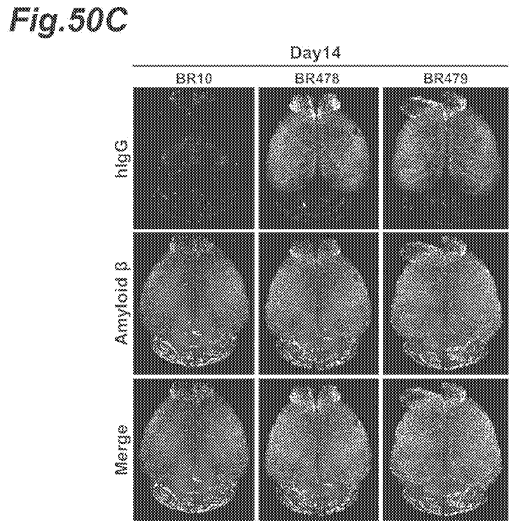

The brain was placed in 50% CUBIC-L solution (CUBICStars, Inc.), immersed overnight at 37° C., and shaken in 100% CUBIC-L solution at 37° C. for 7 days while exchanging liquid every 2 days. After washing with PBS at 37° C. for 2 hours, three times in total, agitation in 1×HV2 buffer (CUBICStars, Inc.) was performed at 37° C. for 1.5 hours. After producing pre-conjugate for each of the primary antibody and secondary antibody at an equivalent amount in molar concentration by shaking at 37° C. for 1.5 hours, Pre-conjugate antibodies, 1×HV2 Buffer (CUBICStars, Inc.), 1/10 Additive A (CUBICStars, Inc.), BOBO-1 staining solution (100.6 μL), brain tissue, and Mounting Solution (CUBICStars, Inc.) were incubated together at 25° C. for 2 weeks. Washing with 1×HV2 Wash Buffer (CUBICStars, Inc.) was performed at 25° C., 3 times in total: for 5 hours, 2 hours, and overnight. Post-fixation was performed at 25° C. for 5 hours with 1% Formaldehyde produced by diluting Formaldehyde (nacalai tesque, Inc., Japan) with 1×HV2 Wash Buffer. The brain was shaken in 10 mM HEPES (TCI) at 25° C. for 2 hours, placed in 50% CUBIC-R (CUBICStars, Inc.), and shaken overnight at 37° C. The brain was shaken in 100% CUBIC-R at 37° C. for 3 days (liquid exchanged on Day 2), and embedded in 2% Agarose/CUBIC-R Gel. The embedded brain tissue was captured with a light sheet microscope (CUBICStars, Inc.) to obtain images, three-dimensional construction was made for comparison. FIGS. 50A to 50C show the distribution of the administered antibodies in the brain and the binding to amyloid plaques. BR478 and BR479 were widely distributed in the brain 3 days after administration, and the binding to amyloid plaques was observed, whereas BR10 localized in a part of the brain. The intracerebral distribution of BR478 and BR479 was observed even 14 days after administration, and the binding to amyloid plaques was maintained.

### Experimental Example 51: Construction of Stable Cell Line (BR802 and BR803)

### (1) Construction of Vector

DNA sequences encoding BR802 and BR803 were inserted into various pPV vectors, similarly to Experimental Example 25. Table 25 shows each region constituting each antibody and the combinations of pPV vectors into which the respective regions were inserted. Based on the combinations described in Table 25, pPV vectors containing the respective antibody regions were fused with pPB vectors to construct expression vectors used for establishing stable cell lines for each antibody.

TABLE 25

| ID | Antibody name | Antibody region | DNA/Amino acid | pPV vector | pPBvector |
|---|---|---|---|---|---|
| BR802 | DI 8-hIgG1-N297G- | First heavy | DNA | pPV-B | pPB- |
| | Hp-HΔK- | chain (H) | | | |
| | aT04m02.1 dsscFv | Second heavy | DNA | pPV-A | QGV |
| | (HF-H-L-L) | chain (HF) | | | |
| | | and dsscFv | | | |
| | | region | | | |
| | | Light chain (L) | DNA | pPV-C | |
| | | | | pPV-D | |
| BR803 | DI 8-hlgG1-N297G- | First heavy | DNA | pPV-B | pPB- |
| | Hp-HΔK- | chain (H) | | | |
| | aT04m0206.1_dsscFv | Second heavy | DNA | pPV-A | QGV |
| | (HF-H-L-L) | chain (HF) | | | |
| | | and dsscFv | | | |
| | | region | | | |
| | | Light chain (L) | DNA | pPV-C | |
| | | | | pPV-D | |

### (2) Construction of Stable Cell Line

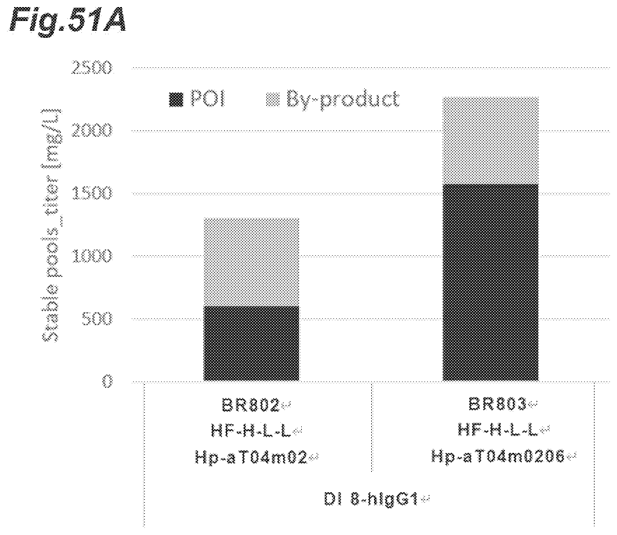

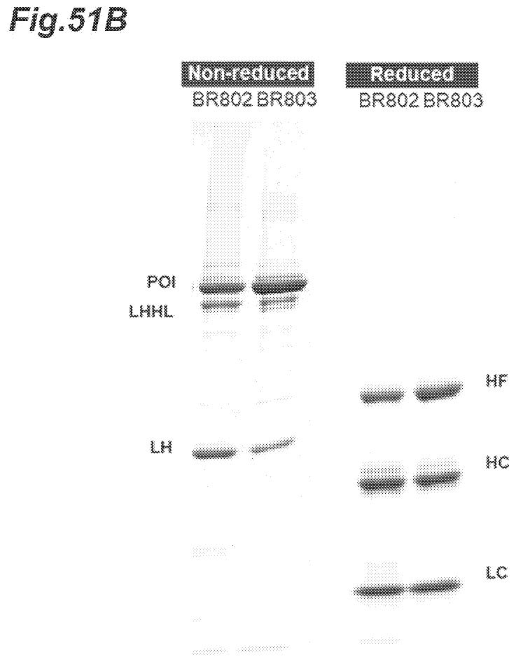

The constructed expression vector was transfected into CHO cells by an electroporation method, and selective culture was performed using a glutamine synthetase inhibitor, MSX (L-Methionine-DL-sulfoximine) in a 50 mL spin tube to establish a stable cell line. The cells were cultured with fed-batch mode for 14 days, and the antibody production including by-products was measured using ProA Biosensor for Octet Red96e (Sartorius AG). The culture supernatant was purified by using MabSelect SuRe resin (Cytiva, Inc.). FIG. 51A shows the antibody production amount by stable cell lines expressing each antibody. Comparing BR802 and BR803, BR803 having aT04m0206 exhibited higher antibody production than BR802 having aT04m02.

### (3) SDS-PAGE

Antibody and SDS sample buffer were mixed at a volume ratio of 1:1, and heated at 70° C. for 10 minutes, and thereby non-reduced samples for SDS-PAGE analysis were prepared. Also, SDS sample buffer containing 10% 3-Mercapto-1, 2-propanediol and the antibody were mixed at a volume ratio of 1:1, and the mixture was heated at 70° C. for 10 minutes, and thereby reduced samples for SDS-PAGE analysis were prepared. These non-reduced and reduced samples were applied on the gel, and the electrophoresis was performed by applying current of 20 mA. After that, the gel was stained with Coomassie Brilliant Blue, and destained with destaining solution (acetic acid:methanol:distilled water=1:1:8). At the end, the gel was scanned with a densitometer (Bio-Rad Laboratories, Inc., GS-900), and then purity evaluation was performed (FIG. 51B). The results of non-reduced SDS-PAGE showed that the POI purity of BR802 was 45.7%, and the POI purity of BR803 was 69.1%. Based on these POI purities, the production amounts of POI shown in FIG. 51A were calculated.

### Experimental Example 52: Separation and Purification

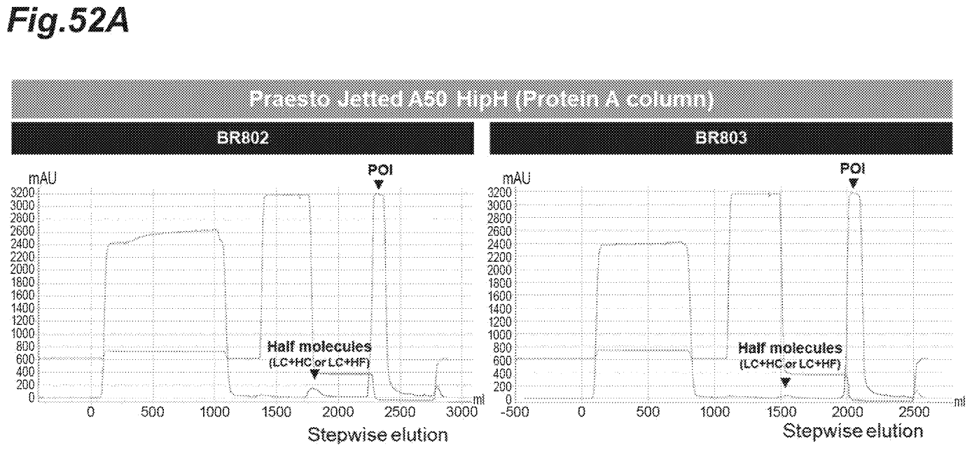

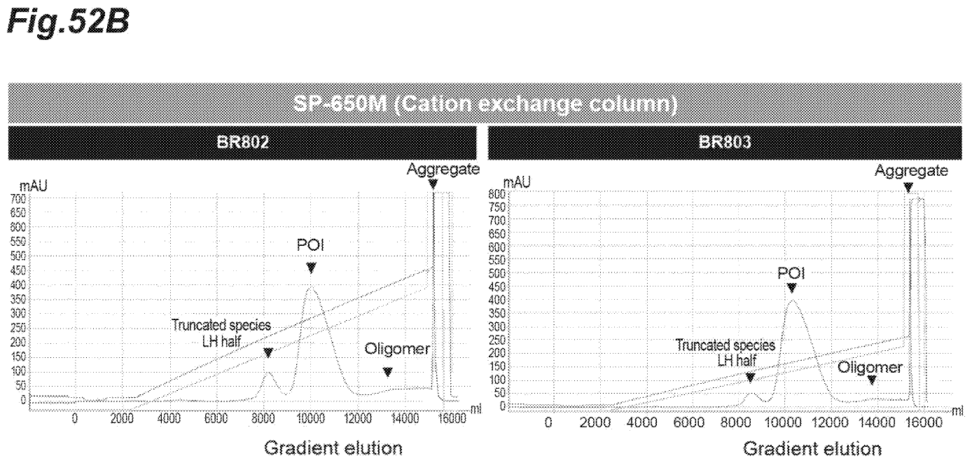

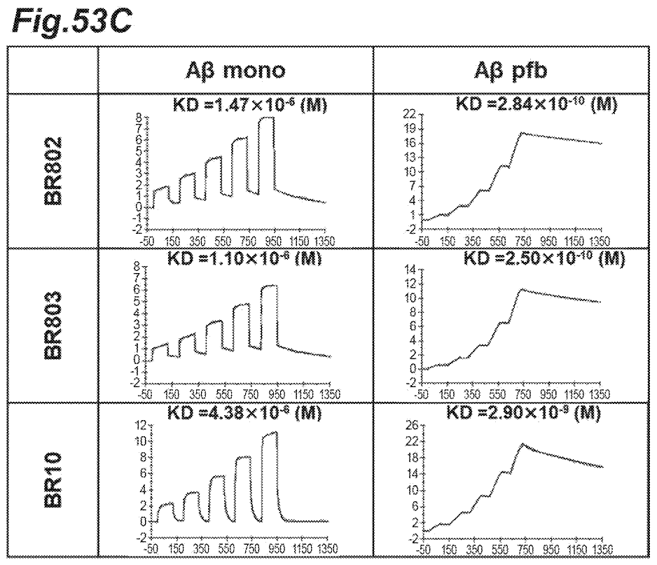

The stable cell lines producing BR802 or BR803 were cultured in a 3 L bioreactor (Able/Biott), and the culture supernatant was obtained. This was subjected to two types of resin columns to purify the target antibodies. In the first-step column purification, a column, Praesto Jetted A50 HipH (Purolite Corporation), filled with resin was used. After the cell culture supernatant of BR802 or BR803 was applied to the Praesto Jetted A50 HipH (Purolite) column, the column was washed with 50 mM phosphate/1 M sodium chloride (pH 7.0), and subsequently washed with 25 mM acetate buffer (pH 5.4). Thereafter, the target antibody was eluted with 25 mM acetate buffer (pH 3.7) (FIG. 52A). The eluate was passed through a Millistak Plus Depth Filter (Millipore). In the second purification step, cation-exchange chromatography was performed using a column packed with SP-650M resin (TOSOH). The depth filtered solution was adjusted to pH 5.5 and applied to the column, and after washing with 25 mM sodium acetate buffer (pH 5.5), the POI were separated from by-products by gradient elution with 25 mM sodium acetate/750 mM sodium chloride buffer (pH 5.5), and the peak containing the POI was collected (FIG. 52B). The eluate was buffer-exchanged with 25 mM acetate buffer (pH 4.8). The purity was confirmed by SDS-PAGE using the final purified product (FIG. 52C). From the results of non-reduced SDS-PAGE, it was revealed that the purity of BR802 was 96.4% and the purity of BR803 was 95.1%, and therefore, it is possible to separate the POI and impurities such as semi-antibodies by this purification method.

### Experimental Example 53: Binding Evaluation to Antigen (Aβ and TfR) (ELISA, SPR, and Flow Cytometry)

### (1) Binding Evaluation to Antigen (Aβ and TfR) (ELISA)

The binding of BR802 and BR803 to protofibril-like Aβ was evaluated according to the ELISA procedures described in Experimental Example 18.

The evaluation of the binding specificity of BR802 and BR803 to protofibrils, and the binding evaluation to human TfR (SEQ ID NO: 243) and monkey TfR (SEQ ID NO: 244), were performed according to the ELISA procedures described in Experimental Example 42.

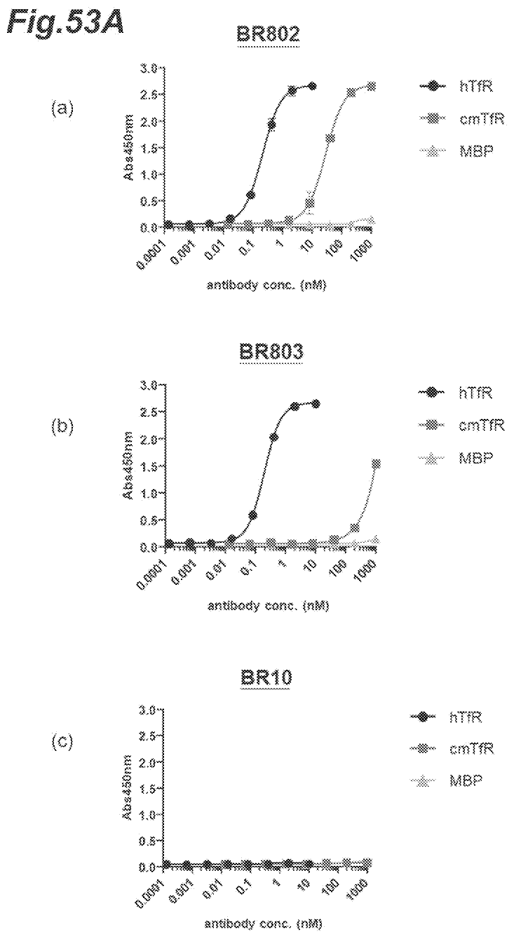

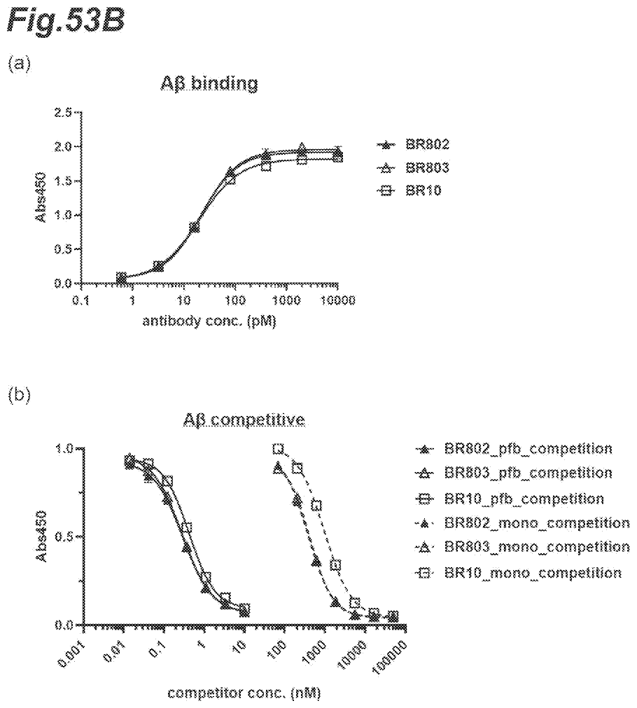

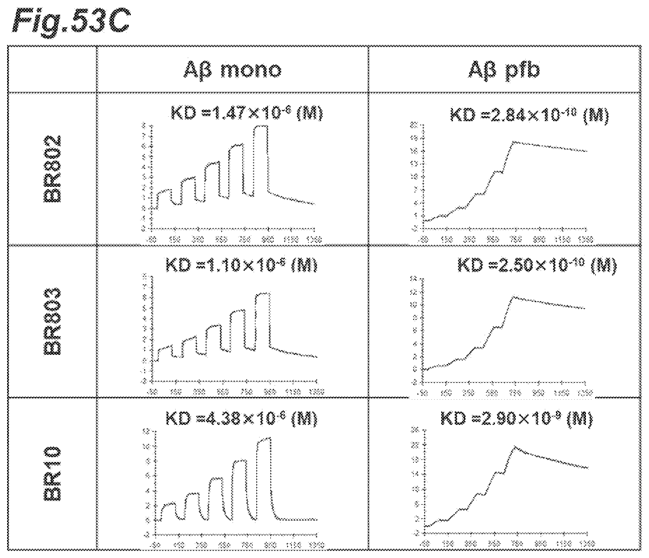

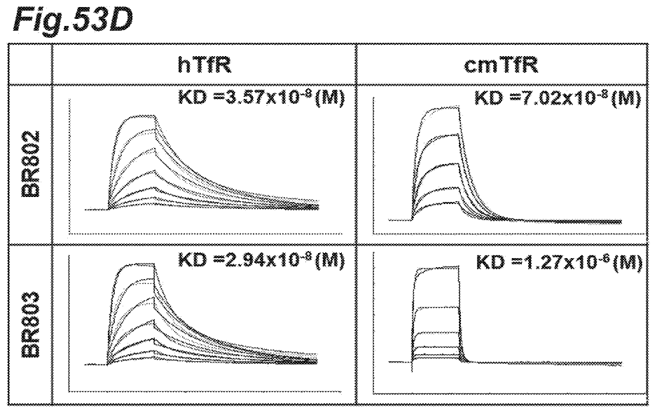

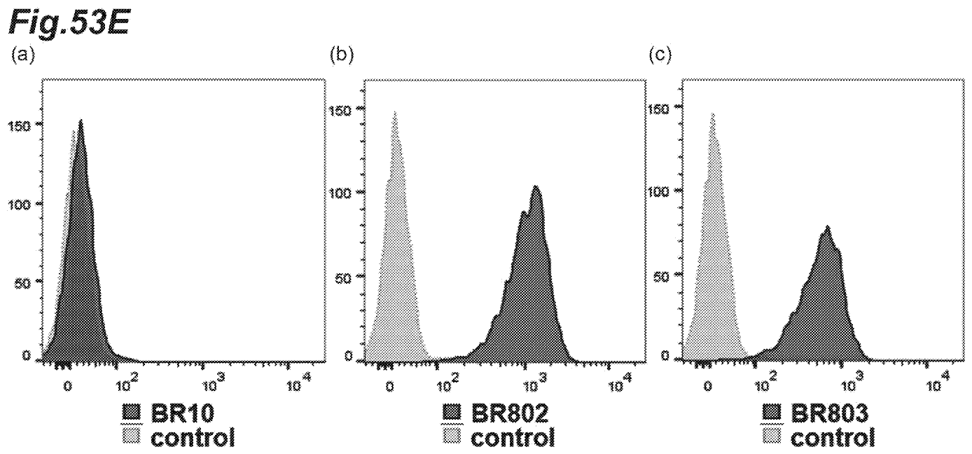

FIGS. 53A and 53B show the absorbance at 450 nm of each antibody concentration. From FIG. 53A, both BR802 and BR803 exhibited equivalent binding properties to human TfR. On the other hand, it was confirmed that the binding property of BR803 to monkey TfR was lower compared with the binding property to human TfR. Similarly, as shown in FIG. 53B, both BR802 and BR803 exhibited equivalent binding properties to protofibril-like Aβ.

### (2) Aβ SPR

The binding affinity of BR802 and BR803 to monomer Aβ and protofibril was measured by surface plasmon resonance (SPR method) according to the procedure described in Experimental Example 18. The calculated KD values and binding response curves are shown in FIG. 53C. As shown in FIG. 53C, BR802 and BR803 exhibit equivalent binding affinity to monomer Aβ (mono) and protofibril (pfb).

### (3) TfR SPR

The binding affinity of BR802 and BR803 to human TfR (SEQ ID NO: 243) and monkey TfR (SEQ ID NO: 244) was measured using surface plasmon resonance (SPR method, Biacore T200, Cytiva, Inc.) according to the procedure described in Experimental Example 18. The calculated KD values and binding response curves are shown in FIG. 53D. The binding KD values of BR802 and BR803 to human TfR (hTfR) were 3.57×10⁻⁸ M and 2.94×10⁻⁸ M, respectively, while the binding KD values to monkey TfR (cmTfR) were 7.02×10⁻⁸ M and 1.27×10⁻⁶ M, respectively. These results indicate that both BR802 and BR803 exhibited comparable binding affinity to human TfR, whereas to monkey TfR, BR802 exhibited a binding affinity approximately 18-fold stronger than BR803. It was confirmed that the binding affinity of BR803 to monkey TfR was lower than the binding affinity of the antibody to human TfR.

### (4) TfR Flow Cytometry

The binding properties of BR10, BR802, and BR803 to U2OS cells were measured according to the following procedures. U2OS cells were washed twice with a washing buffer (1% FBS (Gibco)/1 mM EDTA/PBS), and BR10, BR802, and BR803 diluted in the washing buffer were added to the U2OS cells and incubated on ice for 30 minutes. The cells were washed three times, and a fluorescence-labeled secondary antibody (Jackson ImmunoResearch) diluted in the washing buffer was added and incubated on ice for 30 minutes. The cells were washed three times again, and the fluorescence signal was detected using an LSRFortessa X-20 (BD). The flow cytometry results are shown in FIG. 53E. As shown in FIG. 53E, BR802 and BR803 exhibit equivalent binding properties to U2OS cells.

### Experimental Example 54: Fc-Mediated Effector Function (SPR-FcRs, ADCC, C1q Binding, and In Vitro Amyloid Plaque Phagocytosis)

### (1) SPR-FcRs

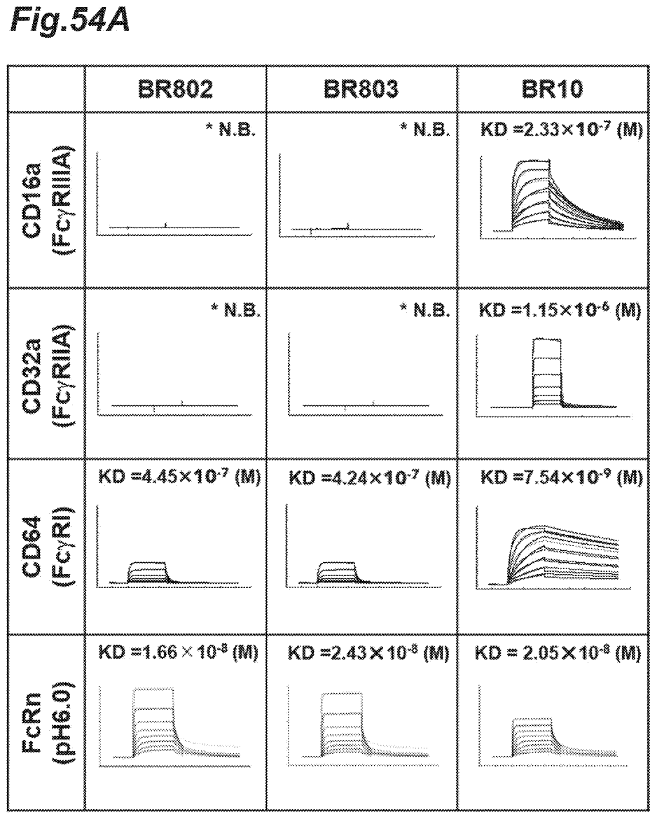

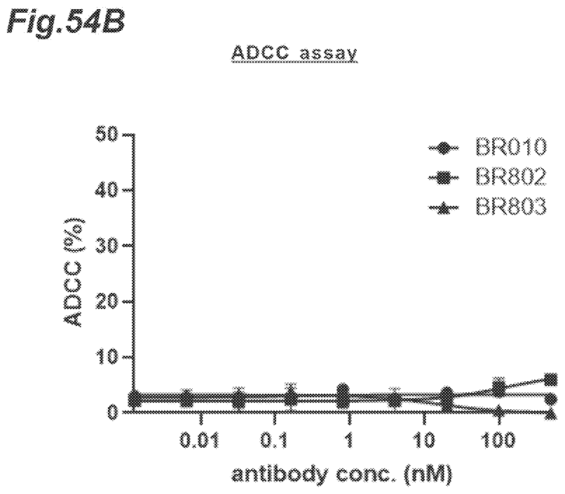

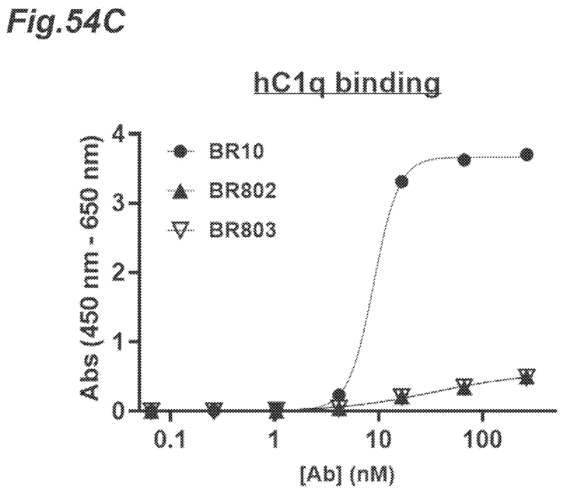

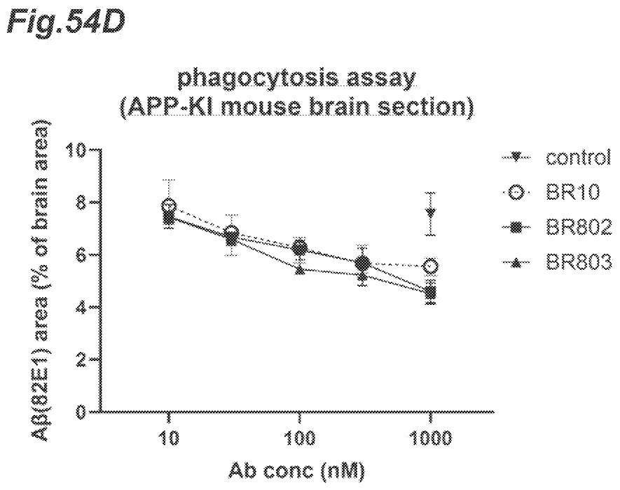

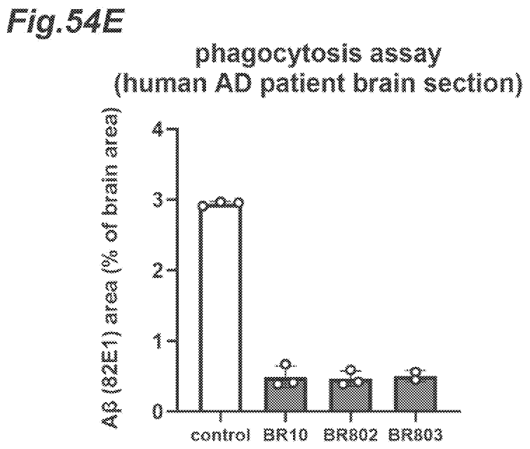

The binding affinities of BR10, BR802 and BR803 to human FcγRI, human FcγRIIA, human FcγRIIIA, and human FcRn were measured using surface plasmon resonance (SPR method, Biacore T200, Cytiva, Inc.) according to the procedure described in Experimental Example 19 (1). The binding response curves and KD values are shown in FIG. 54A. It was suggested from FIG. 54A that BR802 and BR803 having N297G did not show detectable binding properties to human FcγRIIA and human FcγRIIIA, but the binding properties to human FcγRI are attenuated, and the binding properties to human FcRn are retained, compared to BR10.

### (2) Evaluation of Antibody-Dependent Cellular Cytotoxicity (ADCC) Activity

The ADCC activities of BR10, BR802, and BR803 were evaluated using lactate dehydrogenase (LDH) as an indicator, according to the following procedure. U2OS cells were seeded at 6.25×10³ cells/well in 96-well culture plates, using culture medium (10% FBS (NICHIREI CORPORATION)/Kanamycin Sulfate (GIBCO)/McCoy's 5A (GIBCO)), and cultured overnight at 37° C. 5% CO₂. Human PBMC, ADCC-Qualified (Promega Corporation) were thawed and cultured overnight at 37° C. 5% CO₂ in culture medium (10% FBS/1 mM sodium pyruvate (Sigma Aldrich Co. LLC) 5 ng/ml IL-2 (PeproTech, Inc.)/55 μM 2-mercaptoethanol/Antibiotic-Antimycotic (GIBCO)/RPMI-1640 (GIBCO)). On Day 1 of culture, wells containing U2OS cells were washed twice with assay buffer (1% FBS/5 ng/ml IL-2/Antibiotic-Antimycotic/RPMI-1640), and BR10, BR802 and BR803 prepared in assay buffer were added to the plate containing U2OS cells and incubated at 37° C. 5% CO₂ for 30 minutes. Then, PBMC were added at 1.5625×10⁵ cells/well using assay buffer and incubated at 37° C. 5% CO₂ for four hours. After incubation, centrifugation was performed to collect supernatant. An equal volume of Cytotoxicity Detection Kit (Roche) was added to the supernatant, incubated in the dark at room temperature for 30 minutes, and the reaction was stopped with 1N hydrochloric acid, and lactate dehydrogenase (LDH) activity was then measured. Absorbance at 492 nm (with background at 620 nm subtracted) was measured using an EnVision 2105 microplate reader (PerkinElmer, Inc.). LDH absorbance released spontaneously from U2OS cells was defined as (TSR), LDH absorbance released from all U2OS cells lysed with Triton (trademark) X-100 as (TMR), LDH absorbance present in assay buffer as (CMB), LDH absorbance released spontaneously from PBMC as (ESR), and LDH absorbance when Triton (trademark) X-100 was added to assay buffer as (VCC), % ADCC was calculated and shown in FIG. 54B. FIG. 54B indicates that the ADCC activity of BR802 and BR803 having N297G was markedly lower, similar to BR10.

$$\% ADCC ( cytotoxicity ) = [ ( Sample - CMB ) - ( ESR - CMB ) - ( TSR - CMB ) ] / [ ( TMR - VCC ) - ( TSR - CMB ) ] \times 100$$

### (3) C1q Binding

The binding of BR10, BR802, and BR803 to human C1q was measured by ELISA according to the procedure described in Experimental Example 19 (3). The absorbance values at 450 nm, corrected by subtraction of background at 650 nm, are shown in FIG. 54C. As demonstrated in FIG. 54C, BR802 and BR803 having the N297G substitution exhibited markedly reduced binding to human C1q, compared to BR10.

### (4) Evaluation of Fc-Mediated Effector Function (In Vitro Amyloid Plaque Phagocytosis in Brain Tissue of APP KI Mice)

Human monocyte-derived macrophages were prepared as phagocytic cells according to the following procedures. Frozen human peripheral blood mononuclear cells (STEMCELL Technologies) were thawed and added to 10% fetal bovine serum (Gibco)/RPMI 1640 medium (Gibco), and after centrifugation, the cells were suspended in 0.5% BSA/2 mM EDTA/PBS. Classical monocytes were isolated using the Classical Monocyte Isolation Kit human (Miltenyi Biotec) according to the kit's instructions. The isolated cells were suspended in culture medium (10% fetal bovine serum, 2 mM L-Glutamine (Gibco)/Penicillin-Streptomycin (FUJIFILM Wako Pure)/RPMI 1640), and 50 ng/mL GM-CSF (R&D Systems) and 25 ng/ml M-CSF (R&D Systems) at final concentrations, were added. The cells were seeded into a 6-well culture plate, and culture was initiated at 37° C. and 5% CO₂. On Day 3 of culture, an equal volume of culture medium containing 50 ng/ml GM-CSF and 25 ng/mL M-CSF was added. When the cells reached confluence by Day 6 of culture, they were detached using Accutase (Nacalai Tesque) and passaged. On Day 7 of culture, the cells were detached using Accutase, centrifuged, and then suspended in culture medium containing 50 ng/mL GM-CSF and 25 ng/ml M-CSF to a concentration of 1×10⁵ cells/mL, and subjected to a phagocytosis assay. As targets of phagocytosis, 10-month-old female APP KI mice were used. Exsanguination by right auricle incision and transcardial perfusion with heparin-containing PBS were performed, and the brain was collected. The brain was cut in half, fixed at 4° C. overnight in 4% paraformaldehyde phosphate buffer solution (FUJIFILM Wako Pure), then washed with PBS, and frozen in liquid nitrogen. Brain sections at a thickness of 20 μm were prepared using a CM1860 cryostat (Leica), mounted on poly-D-lysine-coated 12 mm coverslips (neu Vitro Corporation), placed in a 24-well plate, air dried at 45° C., and washed with PBS. An antigen retrieval solution prepared by diluting Antigen Retrieval Buffer (100× Tris-EDTA Buffer pH 9.0, abcam) 100 times with distilled water was added, and incubated at 60° C. for 10 hours, followed by washing three times with PBS. Sections were incubated at room temperature for one hour with 10, 30, 100, 300, 1000 nM of BR10, BR802 and BR803, or 1000 nM of control antibody and then washed three times with PBS. Human monocyte-derived macrophages were added at 1×10⁵ cells/well, and cultured at 37° C. in 5% CO₂ for 2 days. After culture, the samples were fixed at room temperature for 30 minutes with 2% paraformaldehyde solution. After washing four times with PBS, blocking was performed at room temperature for 30 minutes with 5% Block Ace (DS Pharma Biomedical Co., Ltd.)/0.1% Tween20/PBS. Anti-Human Amyloid beta (N) (82E1) Mouse IgG MoAb (IBL Co., Ltd.), fluorescently labeled using Alexa Fluor 555 Antibody Labeling Kit (Molecular probes) was diluted with 1% Block Ace and 0.1% Tween20 in PBS, mounted on coverslips, and incubated at room temperature for one hour. After washing three times with PBS, the samples were mounted using Prolong Gold Antifade Mountant (Molecular probes), imaged with NanoZoomer (Hamamatsu Photonics), and the signal of the 82E1 antibody was calculated as the amyloid plaque area using NIS Elements (Nikon). (FIG. 54D). Sections treated with BR10, BR802 and BR803 showed a similar concentration-dependent reduction in anti-Aβ antibody-positive signals.

### (5) Evaluation of Fc-Mediated Effector Function (In Vitro Amyloid Plaque Phagocytosis in Brain Tissue of Alzheimer's Disease Patients)

Human monocyte-derived macrophages were prepared as phagocytic cells according to the following procedures. Frozen human peripheral blood mononuclear cells were thawed and added to 10% fetal bovine serum (Cytiva)/RPMI 1640 medium (Gibco), and after centrifugation, the classical monocytes were isolated according to the procedures of Experimental Example 54 (4). The isolated cells were suspended in culture medium (10% fetal bovine serum, 2 mM L-Glutamine (Gibco)/Penicillin-Streptomycin (FUJIFILM Wako Pure)/RPMI 1640), and 50 ng/mL M-CSF (R&D Systems) at final concentrations were added. The cells were seeded into a 6-well culture plate, and culture was initiated at 37° C. under 5% CO₂. On Day 3 of culture, an equal volume of culture medium containing 50 ng/mL of M-CSF was added. On Day 6 of culture, medium was exchanged to culture medium containing 50 ng/ml M-CSF, 50 ng/mL IFN-γ, and 10 ng/mL LPS. On Day 7 of culture, the cells were detached using Accutase, centrifuged, and then suspended in culture medium containing 50 ng/mL M-CSF and 50 ng/mL IFN-γ to a concentration of 1.2×10⁵ cells/mL, and subjected to a phagocytosis assay. As targets of phagocytosis, fresh frozen brain tissue derived from patients with Alzheimer's disease (ProteoGenex) was used. Brain sections at a thickness of 20 μm were prepared using a CM1860 cryostat (Leica), mounted on poly-D-lysine-coated 12 mm coverslips (neuVitro Corporation) coated with Para-Tissuer (Daido Sangyo Co., Ltd.), placed in a 24-well plate, air dried at 45° C., and washed with PBS. The sections were fixed at room temperature for 30 minutes with 3% paraformaldehyde, and then washed three times with PBS. Sections were incubated at room temperature for one hour with 1000 nM of BR10, BR802 and BR803, or control antibody and then washed three times with PBS. Human monocyte-derived macrophages were added at 1.2×10⁵ cells/well, and cultured at 37° C. in 5% CO₂ for 2 and a half days. After culture, fixation and blocking were performed according to the procedures of Experimental Example 54 (4). Immunostaining was performed at room temperature for 2 hours with Alexa Fluor 555-labeled Anti-Human Amyloid beta (N) (82E1) Mouse IgG MoAb (IBL). As in Experimental Example 54 (4), after washing and mounting, fluorescence images were acquired, and the signal of the 82E1 antibody was calculated as the amyloid plaque area (FIG. 54E). Sections treated with BR10, BR802, and BR803 showed a similar reduction in anti-Aβ antibody-positive signals.

### Experimental Example 55: Mouse PK (hTfR-KI (Hetero) Mouse)

(1) Sample Preparation from Bispecific Anti-human TfR/Aβ human IgG1 Antibody treated Animals

Each of three antibodies of anti-Aβ human IgG1 antibody BR10, and bispecific anti-human TfR/Aβ human IgG1 antibodies BR802 and BR803, was administered as a single intravenous dose to male hTfR-KI (hetero) mice at a dose of 10 mg/kg, calculated as an IgG equivalent amount. Blood was collected under anesthesia at 5 minutes, 6 hours, 1 day, 3 days, 7 days, 14 days, and 28 days after administration. After collecting CSF, exsanguination via incision of the right atrial appendage and transcardial perfusion with heparin-containing PBS were performed, and the brain was harvested and divided into the left and right cerebral hemispheres, and frozen in liquid nitrogen. The blood was centrifuged at 4° C., 1,200×g, for 15 minutes, and the supernatant was collected as plasma. To measure antibody concentrations in the brain, 400 μL of TBS containing complete Protease Inhibitor Cocktail was added to the collection tube of a BioMasher I (Nippi), the filter tube was set and one cerebral hemisphere was added, and the pestle was inserted. After centrifugation at 4° C., 14,000 rpm, for 1 minute, the filter tube was removed, TBS containing complete Protease Inhibitor Cocktail was added so that the total together with the initially added 400 μL would be five times the wet tissue weight, and the mixture was pipetted 10 times. NP-40 Alternative, 10% Solution (Millipore) was added to a final concentration of 1%, and the mixture was incubated at 4° C. with rotation for 1 hour. Centrifugation was performed at 4° C., 15,000 rpm, for 1 minute, and the supernatant was collected as a brain homogenate.

### (2) Measurement of Antibody Concentration

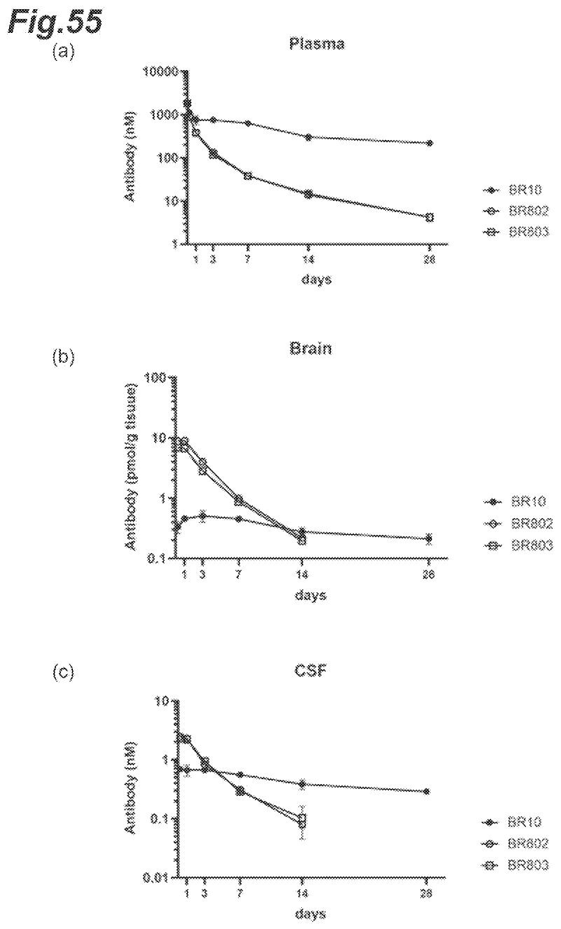

The measurement of the antibody concentrations was performed by ELISA as described below. A donkey anti-human IgG-Fcγ chain-specific F(ab′)2 antibody (Jackson ImmunoResearch Inc.) was added to MaxiSorp Plates (Thermo) and incubated overnight at 4° C. After washing with 0.02% Tween20/PBS three times, blocking was performed at room temperature for one hour by 1% Block Ace (DS Pharma Biomedical Co., Ltd.)/PBS, and then, the brain homogenate, plasma, or CSF diluted in 0.4% Block Ace/PBS were added and incubated at room temperature for 2 hours. After washing with 0.02% Tween20/PBS three times, an HRP-labeled goat anti-human IgG Fcγ chain-specific F(ab′)2 antibody (Jackson ImmunoResearch Inc.) was added and incubated at room temperature for one hour. After washing with 0.02% Tween20/PBS three times, TMB substrate solution (SeraCare) was added and incubated at room temperature for 30 minutes. An equivalent amount of 2N sulfuric acid was added to quench the reaction, and the absorbance at 450 nm and 650 nm was measured with a microplate reader SpectraMax ABS (Molecular Devices, LLC). Calibration curves were generated based on the absorbance of standard samples at each concentration, and the antibody concentrations of the respective samples were determined (FIG. 55, Table 26). It was indicated that the antibody concentrations in the brain and CSF at 6 hours and 1 day post-administration were remarkably higher in the bispecific antibodies BR802 and BR803 than for BR10. For the antibody concentrations in the brain, plasma, and CSF, there were no marked differences observed between the bispecific antibodies BR802 and BR803.

TABLE 26

| | | | | BR10 | BR802 | BR803 |
|---|---|---|---|---|---|---|
| IV | Plasma | AUC_{inf} | μg*hr/mL | 56681.2 | 7796.6 | 7605.5 |
| | | T_{1/2} | hr | 331 | 162 | 162 |
| | Brain | AUC_{inf} | ng*hr/mL | 53672.2 | 137143.9 | 106381.9 |
| | CSF | AUC_{inf} | ng*hr/mL | 77314.6 | 35980.4 | 37130.4 |
| | | Brain/Plasma | % | 0.09 | 1.76 | 1.40 |
| | | AUC Ratio | | | | |
| | | CSF/Plasma | % | 0.14 | 0.46 | 0.49 |
| | | AUC Ratio | | | | |

### Experimental Example 56: Reticulocyte Deletion (hTfR-KI (Hetero) Mice)

(1) Sample Preparation from Animals Given Bispecific Anti-Human TfR/Aβ Human IgG1 Antibody

Three antibodies of anti-Aβ human IgG1 antibody BR10, and the bispecific anti-human TfR/Aβ human IgG1 antibodies BR802 and BR803 were intravenously administered to male hTfR-KI (hetero) mice at a single dose of 10 mg/kg, calculated as an IgG equivalent amount, and the blood was collected under anesthesia at 5 minutes, 6 hours, 1, 3, 7, 14, and 28 days post-administration.

### (2) Blood Test

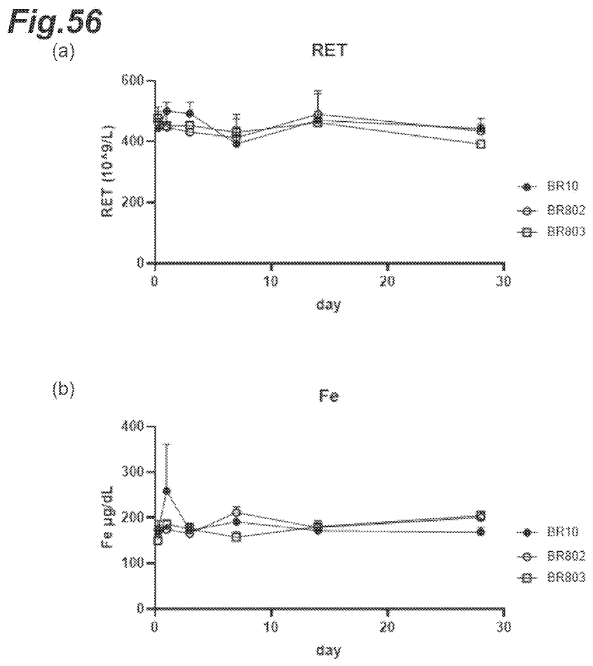

Hematology test and blood chemistry test were performed using the blood samples obtained by using Capiject II (Terumo Corporation) in the above (1). Also, nitroso-PSAP method was used for the iron measurement in the plasma. As a result, there were no toxicologically meaningful changes in reticulocyte (RET) and iron for both antibodies BR802 and BR803 (FIG. 56).

### Experimental Example 57: Effect of Single Administration on Oligomer Aβ in Brain Tissue (APP/hTfR-KI (Homo/Hetero) Mice)

### (1) Establishment of APP-KI/hTfR-KI (Homo/Hetero) Mice

APP KI/hTfR-KI (homo/hetero) mice were generated by intercrossing APP KI/hTfR-KI (homo/homo) mice generated in Experimental Example 14 with APP KI mice (RBRC06344, RIKEN) in which amyloid plaques are formed in the brain. The resulting APP-KI/hTfR-KI (homo/hetero) mice were bred and maintained.

(2) Sample Preparation from Animals Given Bispecific Anti-Human TfR/Aβ Human IgG1 Antibody

7-month-old male APP KI/hTfR-KI (homo/hetero) mice were administered the anti-Aβ human IgG1 antibody BR10 at a dose of 30 mg/kg and the bispecific anti-human TfR/Aβ human IgG1 antibodies BR802 and BR803 at doses of 10 mg/kg and 30 mg/kg, calculated as an IgG equivalent amount by intravenous injection via the tail vein. In the control group, 25 mM sodium acetate buffer (pH 4.8) was administered by intravenous injection via the tail vein. One week after the final administration, blood was collected under anesthesia, followed by exsanguination via right atrial appendage incision and transcardial perfusion with PBS containing heparin. The brain was harvested and sectioned into left and right cerebral hemispheres. The right cerebral hemisphere was weighed to determine wet tissue weight, frozen in liquid nitrogen, and stored at −80° C. 10 times the amount of the wet tissue weight of TBS was added and tissue was homogenized using a homogenizer (BRANSON, Inc.), and then, centrifugation was performed at 16,000×g for 60 minutes at 4° C., and the supernatant was collected as TBS soluble fraction (16k_sup). The obtained supernatant was further centrifuged at 100,000×g for 60 minutes at 4° C., and the supernatant was collected as the TBS-soluble fraction (16k/100k_sup). The collected blood was centrifuged at 1,200×g for 15 minutes at 4° C., and the supernatant was collected as plasma.

### (3) Measurement of Antibody Concentration

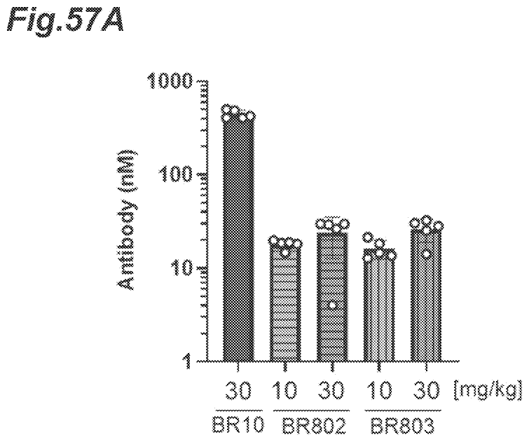

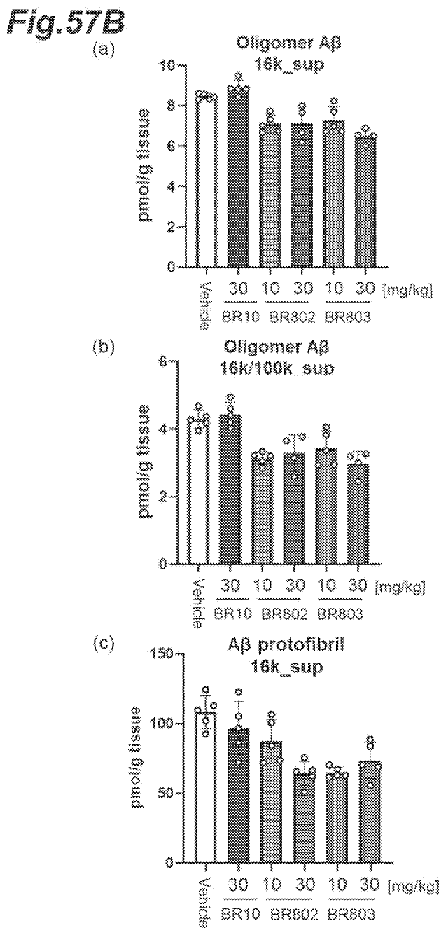

The measurement of the antibody concentrations was performed by ELISA as described below. A donkey anti-human IgG-Fcγ chain-specific F(ab′)2 antibody (Jackson ImmunoResearch Inc.) was added to MaxiSorp Plates (Thermo) and incubated overnight at 4° C. After washing with 0.02% Tween20/PBS three times, blocking was performed at room temperature for one hour by 1% Block Ace (DS Pharma Biomedical Co., Ltd.)/PBS, and then, plasma diluted using 0.4% Block Ace/PBS were added and incubated at room temperature for 2 hours. After washing with 0.02% Tween20/PBS three times, an HRP-labeled goat anti-human IgG Fcγ chain-specific F(ab′)2 antibody (Jackson ImmunoResearch Inc.) was added and incubated at room temperature for one hour. After washing with 0.02% Tween20/PBS three times, TMB substrate solution (SeraCare) was added and incubated at room temperature for 30 minutes. An equivalent amount of 2N sulfuric acid was added to quench the reaction, and the absorbance at 450 nm and 650 nm was measured with a microplate reader SpectraMax ABS (Molecular Devices, LLC). Calibration curves were created based on the absorbance of standard samples at each concentration, and the antibody concentrations of the respective samples were determined (FIG. 57A). Individuals with markedly low antibody concentrations in plasma samples were excluded from the analysis. The antibody concentration thresholds set for each administration group were as follows: 5 nM (group administered BR802 or 803 at 10 mg/kg) and 15 nM (group administered BR802 or BR803 at 30 mg/kg).

### (4) Measurement of Oligomer Aβ Concentrations in TBS-Soluble Fraction

For the TBS-soluble fraction, the reaction was performed using Human Amyloidβ Oligomers (82E1-specific) Assay Kit (IBL Co., Ltd.) according to the instructions in the kit. After the reaction, the absorbance at 450 nm was measured with a microplate reader SpectraMax (Molecular Devices, LLC). The concentrations of oligomer Aβ included in the TBS soluble fraction were determined, and further, the amount of oligomer Aβ per g of wet brain tissue was calculated (FIG. 57B). In FIG. 57B(a) and (b), in both 16k_sup and 16k/100k_sup, no reduction in oligomer Aβ was observed in the group administered BR10 at 30 mg/kg compared to the control group, however, reductions in oligomer Aβ were observed in the group administered BR802 or BR803 at 10 mg/kg or 30 mg/kg. No marked difference in the decrease of oligomeric Aβ was observed between BR802 and BR803.

### (5) Measurement of Aβ Protofibril Concentrations in TBS-Soluble Fraction

The Aβ protofibril-capturing antibody BR10 was labeled onto magnetic beads to prepare antibody-labeled magnetic beads. The prepared antibody-labeled magnetic beads were diluted with Discovery/Assay Buffer (EMD Millipore) to a concentration of 100 μg/mL. Fifty microliters of the antibody-labeled bead solution and 150 μL of TBS-soluble fraction 16k_sup diluted with Discovery Standard Diluent (EMD Millipore) were added to each well of a 96-well V-bottom plate (Axygen), and the plate was shaken at 27° C. and 800 rpm for 2 hours. After washing the 96-well V-bottom plate with washing buffer, a fluorescent dye-labeled anti-Aβ antibody, mouse 3D6 antibody (ADx Neurosciences), was added to each well, followed by shaking at 27° C. and 800 rpm for 1 hour. After washing the 96-well V-bottom plate with washing buffer, 12 μL of Elution Buffer B (EMD Millipore) was added to each well, and the plate was shaken at 27° C. and 800 rpm for 10 minutes. After shaking, 10 μL was collected from each well of the plate and added to each well of a 384-well plate (Aurora Microplates) to which 10 μL of Buffer D (EMD Millipore) had been added in advance. The 384-well plate was shaken at 25° C. and 1000 rpm for 2 minutes, and fluorescence derived from the fluorescently labeled antibody was counted using SMCxPRO (EMD Millipore), and the Aβ protofibril concentration was calculated. The quantification results were normalized to wet brain weight (FIG. 57B (c)). Compared with the control group, no reduction in Aβ protofibrils was observed in the group administered 30 mg/kg of BR10, whereas a reduction in Aβ protofibrils was observed in the group administered 30 mg/kg of BR802. In addition, a tendency toward reduction in Aβ protofibrils was observed in the groups administered 10 mg/kg or 30 mg/kg of BR803.

### Experimental Example 58: Monkey PK

(1) Sample Preparation from Animals Given Bispecific Anti-Human TfR/Aβ Antibody

Two antibodies, BR802 and BR803 were each intravenously administered to male cynomolgus monkeys at a dose of 5 and 30 mg/kg, and the peripheral blood was collected before administration, and at 5 minutes, 2, 6, 24, 96, 168, 336, 504, and 600 hours post-administration to separate the serum. Also, CSF was collected at 24, 96, 168, 336, 504, and 600 hours post-administration, and, along with the serum, the measurement of concentrations of humanized anti-hTfR antibodies in (2) below was performed.

### (2) Measurement of Concentration of Bispecific Anti-Human TfR/Aβ Antibody in Serum and CSF

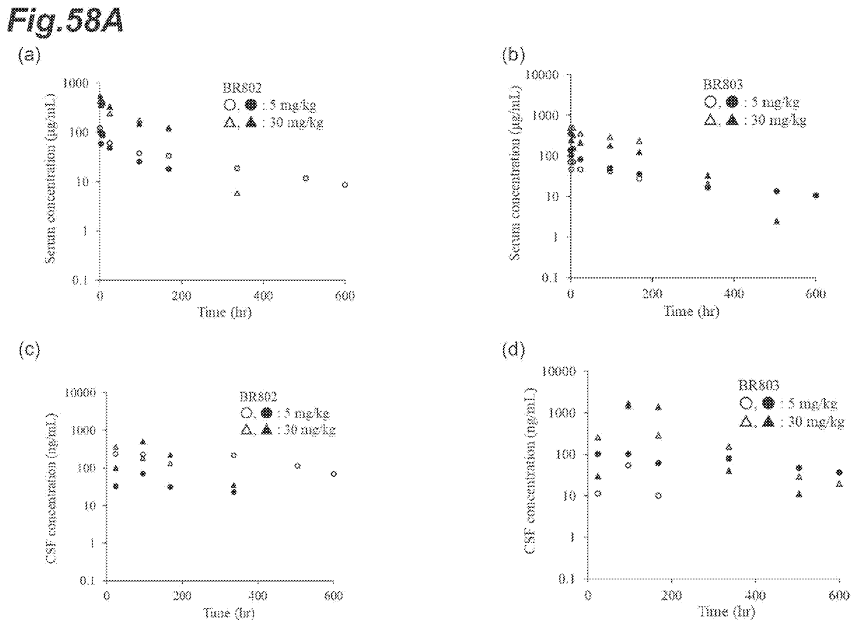

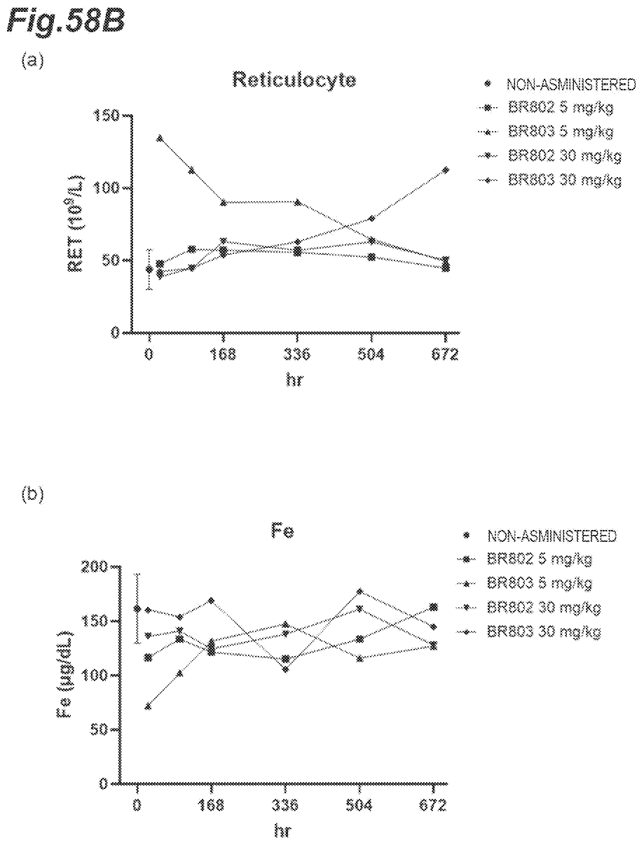

To measure the concentrations of the administered antibodies in cynomolgus monkey serum, diluted serum samples were added to the plate in which monomer Aβ 1-40 (Anaspec, Inc.) was immobilized, an HRP-labeled anti-human IgG antibody was added and incubated, and then, TMB substrate solution was added and incubated, and sulfuric acid was added to complete the assay. By measuring the absorbance at 450 nm and 650 nm of the liquids on the plate and creating calibration curves based on the absorbance of standard samples at each concentration, the antibody concentrations of the respective samples were determined. By the assay, calibration curve in the range of 2.5 to 500 μg/ml for the serum was obtained. To measure antibody concentrations in CSF, diluted CSF samples were added to the plate in which anti-human Kappa Light Chain Goat IgG (Immuno-Biological Laboratories, Inc.) was immobilized, and SULFO-Tag-labeled anti-human IgG antibody was added and incubated, and then Read Buffer was added to complete the assay. By measuring the signal from the liquids on the plate using an electrochemiluminescence detector and creating calibration curves based on the luminescence signals of standard samples at each concentration, the antibody concentrations of the respective samples were determined. By the assay, calibration curve in the range of 15.625 to 1000 ng/ml for CSF was obtained. The results less than the detection limit were reported as BQL. FIG. 58A shows pharmacokinetic profiles of BR802 and BR803. For the transition of concentrations in serum after the administration, there were no marked differences observed between the bispecific antibodies BR802 and BR803. For the concentrations in CSF, there were no marked differences observed between BR802 and BR803 at 5 mg/kg, whereas BR803 exhibited high values at 30 mg/kg. Symbols (white circle, black circle, white triangle, and black triangle) in FIG. 58A indicate the transition of concentrations for each individual.

### (3) Blood Test

Hematology test and blood chemistry test were performed using the blood samples of before administration, and at 24, 96, 168, 336, 504, and 600 hours post-administration in (1) above. Also, nitroso-PSAP method was used for the iron measurement in serum. As a result, neither changes suggesting dose dependency for both BR802 and BR803, nor clear changes attributable to both the antibodies, in reticulocyte (RET) and iron was observed (FIG. 58B).

### Experimental Example 59: Binding to Amyloid Plaque (APP KI/hTfR-KI Mice)

(1) Sample Preparation from Animals Given Bispecific Anti-Human TfR/Aβ Human IgG1 Antibody

To 6-month-old male APP KI/hTfR-KI (homo/hetero) mice, the anti-Aβ human IgG1 antibody BR10 at a dose of 10 mg/kg, and the bispecific anti-human TfR/Aβ human IgG1 antibodies BR802 and BR803 at dose of 10 mg/kg, calculated as an IgG equivalent amount were administered via the tail vein (10 mL/kg). At 1, 3, 7, and 14 days post-administration, exsanguination via incision of the right atrial appendage followed by transcardial perfusion with PBS containing heparin was performed, and transcardial perfusion with 4% paraformaldehyde/phosphate buffer solution (PB) was performed in a similar route to harvest the brain. The collected brain was placed in 4% paraformaldehyde/PB, and immersed and fixed at 4° C. for 24 hours, and then the fixed brain was transferred to PBS and immersed therein.

### (2) Tissue Clearing and Whole-Mount-IHC

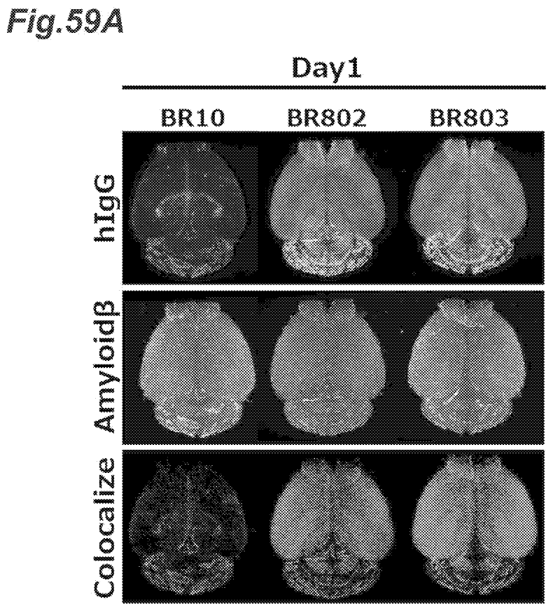

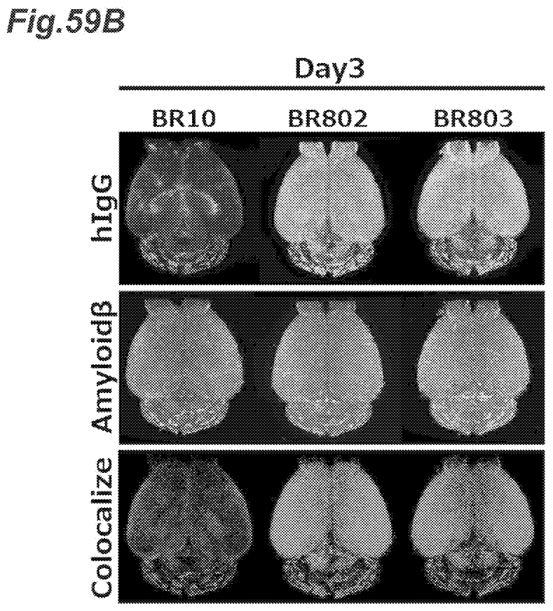

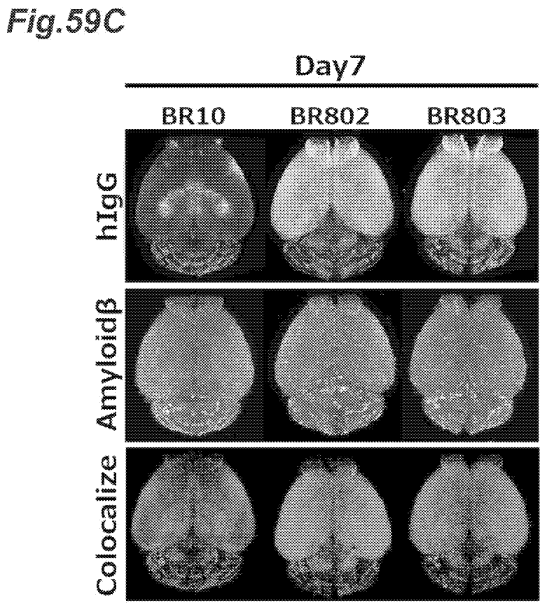

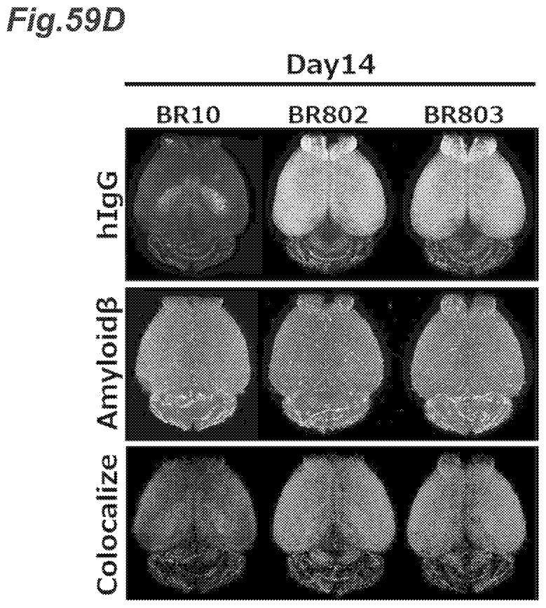

The brain was placed in 50% CUBIC-L solution (CUBICStars, Inc.), immersed overnight at 37° C., and shaken in 100% CUBIC-L solution at 37° C. for 7 days while exchanging liquid every 2 days. After washing with PBS at 37° C. for 2 hours, three times in total, agitation in 1×HV2 buffer (CUBICStars, Inc.) was performed at 37° C. for 1.5 hours. After producing pre-conjugate for each of the primary antibody and secondary antibody at an equivalent amount in molar concentration by shaking at 37° C. for 1.5 hours, Pre-conjugate antibodies, 1×HV2 Buffer (CUBICStars, Inc.), 1/10 Additive A (CUBICStars, Inc.), BOBO-1 staining solution (100.6 μL), brain tissue, and Mounting Solution (CUBICStars, Inc.) were incubated together at 25° C. for 2 weeks. Washing with 1×HV2 Wash Buffer (CUBICStars, Inc.) was performed at 25° C., 3 times in total: for 5 hours, 2 hours, and overnight. Post-fixation was performed at 25° C. for 5 hours with 1% Formaldehyde produced by diluting Formaldehyde (nacalai tesque, Inc., Japan) with 1×HV2 Wash Buffer. The brain was shaken in 10 mM HEPES (TCI) at 25° C. for 2 hours, placed in 50% CUBIC-R (CUBICStars, Inc.), and shaken overnight at 37° C. The brain was shaken in 100% CUBIC-R at 37° C. for 3 days (liquid exchanged on Day 2), and embedded in 2% Agarose/CUBIC-R Gel. The embedded brain tissue was captured with a light sheet microscope (CUBICStars, Inc.) to obtain images, three-dimensional construction was performed for comparison. FIGS. 59A to 59C show the distribution of the administered antibodies in the brain and the binding to amyloid plaque. BR802 and BR803 widely distributed in the brain 1 day after administration, and the binding to amyloid plaques was observed, whereas BR10 localized in a part of the brain. The intracerebral distribution of BR802 and BR803 was observed even 14 days after administration, and the binding to amyloid plaques was maintained.

### Experimental Example 60: Evaluation of ARIA Risk

(1) Sample Preparation from Animals Given Bispecific Anti-Human TfR/Aβ Human IgG1 Antibody

The ARIA risk of the bispecific anti-human TfR/Aβ human IgG1 antibody BR803 was evaluated by administering BR803 to 5xFAD/hTfR-KI (hemi/homo) mice and, as in Experimental Example 15, quantifying and analyzing the number of infiltrating peripheral immune cells in the mouse brain tissue. Specifically, male 5xFAD/hTfR-KI (hemi/homo) mice aged 8 months were administered anti-Aβ human IgG1 antibody BR847 known to be associated with ARIA risk in clinical trials (humanized 3D6 antibody (3D6v2), an anti-Aβ antibody disclosed in WO2002/046237, and is also known as bapineuzumab; hereinafter referred to as “BR847”) (Reisa Sperling et al. The Lancet Neurology March 2012, 11 (3) 241-249) at a dose of 100 mg/kg, or bispecific anti-human TfR/Aβ human IgG1 antibody BR803 at a dose of 10 mg/kg or 30 mg/kg IgG, calculated as an IgG equivalent amount by intravenous injection via the tail vein (10 mL/kg). The control group received 25 mM sodium acetate (pH 5.0) by intravenous injection via the tail vein. Fifteen hours after the administration, blood was collected under anesthesia, followed by exsanguination via right auricle incision and transcardial perfusion with heparin-containing PBS were performed to harvest the brain. The brain was divided into left and right cerebral hemispheres. The left cerebral hemisphere was immersed and fixed for 24 hours at 4° C. in 1% paraformaldehyde (TAAB Laboratories Equipment Ltd.)/PBS. It was substituted with 10% sucrose/PBS and 20% sucrose/PBS, and then, embedded in Tissue-Tek O. C. T. Compound (Leica) to freeze in liquid nitrogen.

### (2) Evaluation of ARIA Risk Using Immune Cell Infiltration as Index

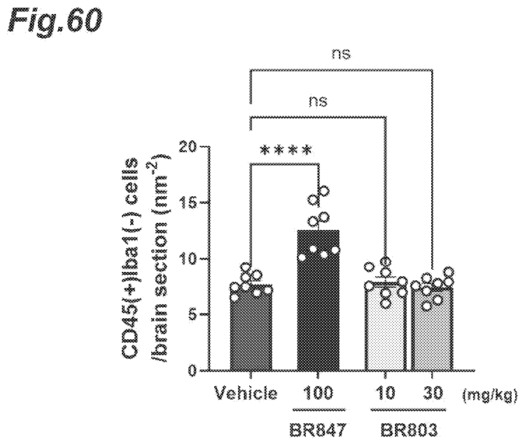

The prepared O. C. T. Compound-embedded and frozen tissue was sectioned in a thickness of 10 μm using Cryostat CM1860 (Leica), a section was mounted on a glass slide, and then air dried under cold airflow, enclosed in a sealed bag and stored at −80° C. The glass slide was washed with PBS, immersed in 1% normal donkey serum (Jackson ImmunoResearch Inc.)/0.1% Triton (trademark) X-100/25% Block Ace solution, and subjected to blocking procedure at room temperature for one hour. CD45 antibody (R&D Systems, Inc.) and Iba1 antibody (FUJIFILM Wako Pure) diluted in PBS containing 0.1% normal donkey serum (Jackson ImmunoResearch Inc.)/0.1% Triton (trademark) X-100/2.5% Block Ace solution were incubated at room temperature for two hours. After performing washing procedure three times with 0.1% Triton (trademark) X-100/PBS solution for 5 minutes, a fluorescent-labeled secondary antibody (Jackson ImmunoResearch Inc.) was incubated at room temperature for one hour, and then, cell nucleus staining (Life Technologies Hoechst 33342) was incubated at room temperature for 5 minutes. After performing washing procedure three times with 0.1% Triton (trademark) X-100/PBS solution for 5 minutes, the mixture was washed with PBS once, and then, Prolong Gold antifade reagent (Thermo) was placed on the section and sealed, and observed by Nanozoomer S60 (Hamamatsu Photonics K.K.) to obtain images. The number of infiltrating CD45+ Iba1− peripheral immune cells in the brain tissue was quantified and analyzed using NIS elements (Nikon Corporation). For the obtained data, one-way analysis of variance (one-way ANOVA) was performed, followed by Tukey's multiple comparison test to compare with the control group. As a result, it was found that, compared with the control group, the anti-Aβ antibody BR847 had a statistically significantly increased number of infiltrating peripheral immune cells, whereas the bispecific anti-human TfR/Aβ antibody BR803 did not statistically significantly changed the number of infiltrating peripheral immune cells (FIG. 60). “****” in FIG. 60 indicates that the P value between groups is less than 0.0001.

### Experimental Example 61: Production of Monoclonal Antibodies (Transient Expression)

### (1) Construction of Bispecific Anti-Human TfR/Aβ Mouse IgG2a Chimeric Antibody Gene Expression Vector

In the vector expressing the first heavy chain, and the second heavy chain and scFv genes of BR479 (SEQ ID NOs: 189 and 193), DNA sequences were synthesized in which the amino acid sequence encoding the human IgG1 constant region was replaced with the amino acid sequence encoding the mouse IgG2a constant region, and in which aT04m0206-scFv was further replaced with aT04m0206-dsscFv. These DNA sequences were cloned into a pcDNA3.4 vector. Accordingly, vectors expressing the first heavy chain gene (DNA sequence: SEQ ID NO: 198; amino acid sequence: SEQ ID NO: 199) and the second heavy chain and dsscFv gene (DNA sequence: SEQ ID NO: 200; amino acid sequence: SEQ ID NO: 201) of the bispecific antibody containing the mouse IgG2a constant region were constructed (BR586).

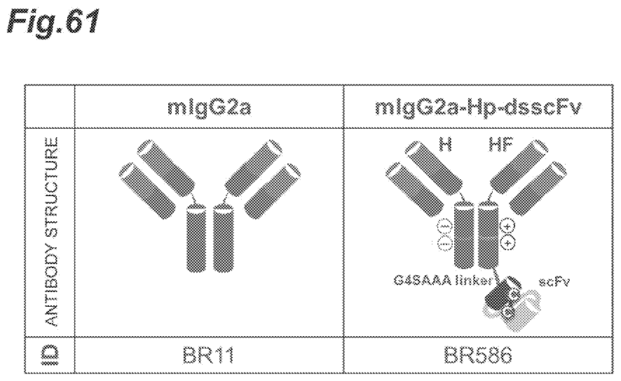

Schematic diagrams of BR11 and BR586, and the combinations of the first and second heavy chains and the light chains included in BR11 and BR586 are shown in FIG. 61 and Table 27, respectively. Hereinafter, in the tables, “E356K” refers to a mutation in mouse IgG2a corresponding to the D356K substitution in human IgG1, and means the substitution of glutamic acid at residue 356 according to EU numbering in mouse IgG2a with lysine.

TABLE 27

| | | | DNA/ | SEQ |
|---|---|---|---|---|
| | | Antibody | Amino | ID |
| ID | Antibody name | region | acid | NO: |
| BR586 | DI 8-mlgG2a-Hp- | First heavy | DNA | 198 |
| | aT04m0206.1-dsscFv | chain (H) | Amino | 199 |
| | H; K409D, K439E | | acid | |
| | HF; D356K, D399K, | Second heavy | DNA | 200 |
| | linker (G4SAAA), | chain (HF) | Amino | 201 |
| | | and dssc Fv | acid | |
| | | region | | |
| | aT04m0206-dsscFv | Light chain (L) | DNA | 100 |
| | L; wt | | Amino | 101 |
| | | | acid | |

### (2) Production and Purification of Bispecific Anti-Human TfR/Aβ Mouse IgG2a Chimeric Antibody

The bispecific anti-human TfR/Aβ mouse IgG2a chimeric antibody BR586 was transiently produced according to the manufacturer's high-titer protocol using an ExpiCHO expression system (Thermo). The cell culture medium containing the secreted antibodies was collected 8 days after transfection. The clarified supernatant, obtained by removing cells by centrifugation, was purified using a MabSelect SuRe resin (Cytiva, Inc.) and eluted with 100 mM citrate buffer (pH 3.8) containing 500 mM arginine hydrochloride. After adjusting the pH of the antibody solution, the antibody solution was concentrated using a centrifugal ultrafiltration filter and diluted 20-fold with 25 mM sodium acetate buffer (pH 5.6). Next, in order to separate byproducts, the mixture was applied to a cation exchange column, HiTrap SP HP 5 mL (Cytiva, Inc.). After washing the column, the byproducts and protein of interest (POI) were separated by gradient elution using 25 mM sodium acetate/750 mM sodium chloride buffer (pH 5.6). Fractions containing the POI were collected and buffer-exchanged with PBS by dialysis.

### Experimental Example 62: Evaluation of Dose Reduction of a Bispecific Antibody (APP KI/hTfR-ApD-KI Mouse)

### (1) Establishment of APP KI/hTfR-ApD-KI Mice

Similarly to Experimental Example 48, APP-KI/hTfR-ApD-KI (homo/homo) mice were generated.

(2) Sample Preparation from Animals Given Bispecific Anti-Human TfR/Aβ Mouse IgG2a Antibody

Male APP KI/hTfR-ApD-KI (homo/homo) mice at 6 months of age were administered the anti-Aβ mouse IgG2a antibody BR11 at a dose of 10 mg/kg, and the bispecific anti-human TfR/Aβ mouse IgG2a antibody BR586 at doses of 0.3 mg/kg, 1.0 mg/kg, and 3.0 mg/kg, calculated as an IgG equivalent amount, once a week for 12 weeks by intraperitoneal injection (10 mL/kg). PBS was administered to the control group. One week after the final administration, blood was collected under anesthesia, followed by exsanguination via incision of the right atrial appendage and transcardial perfusion with heparin-containing PBS. The brain was harvested and sectioned into left and right cerebral hemispheres. The left cerebral hemisphere was immersed in 1% paraformaldehyde (TAAB Laboratories Equipment Ltd.)/PBS and fixed at 4° C. for 24 hours. The fixed left cerebral hemisphere was sequentially replaced with 10% sucrose/PBS and 20% sucrose/PBS, and embedded in Tissue-Tek O. C. T. Compound (Leica) and frozen in liquid nitrogen to prepare a cryosection block. The right cerebral hemisphere was weighed to determine wet tissue weight, frozen in liquid nitrogen, and stored at −80° C. The collected blood was centrifuged at 1,200×g for 15 minutes at 4° C., and the supernatant was collected as plasma.

### (3) Measurement of Antibody Concentration in Plasma

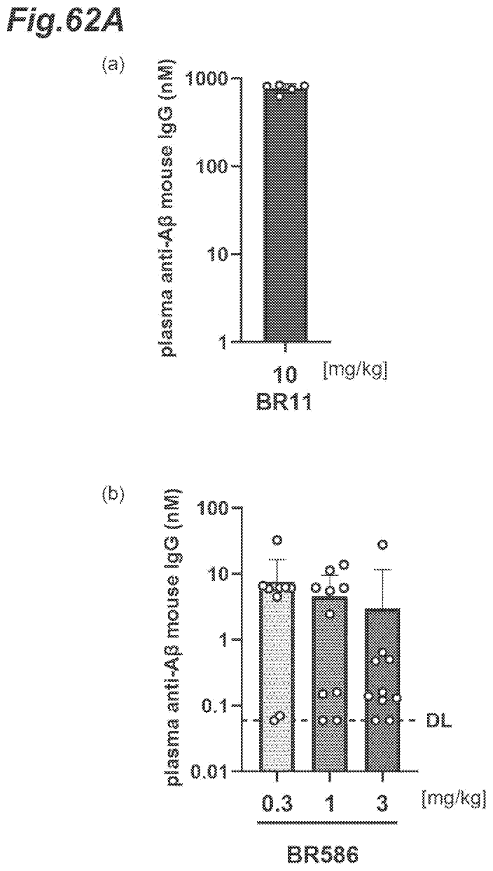

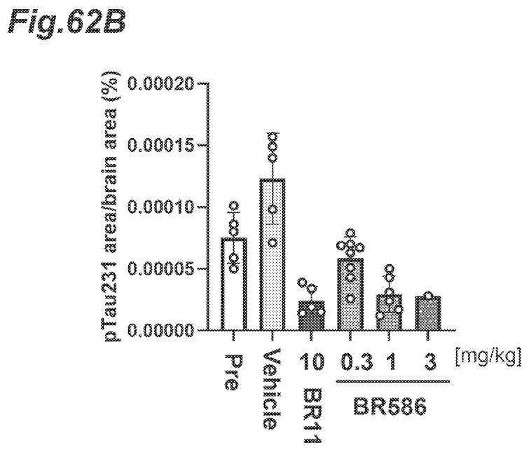

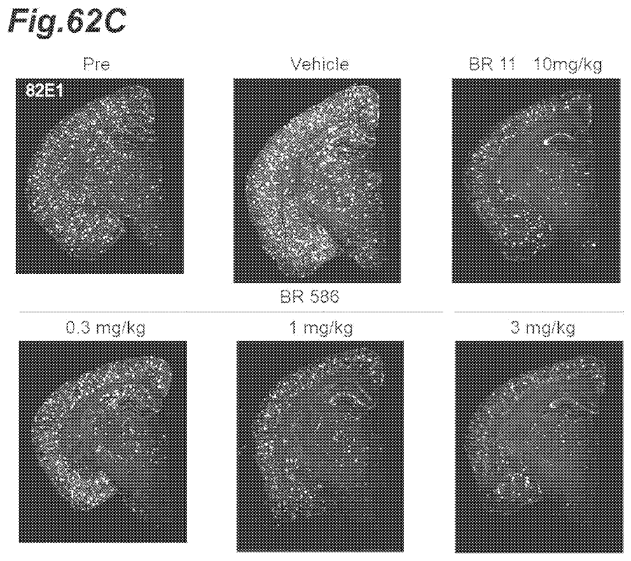

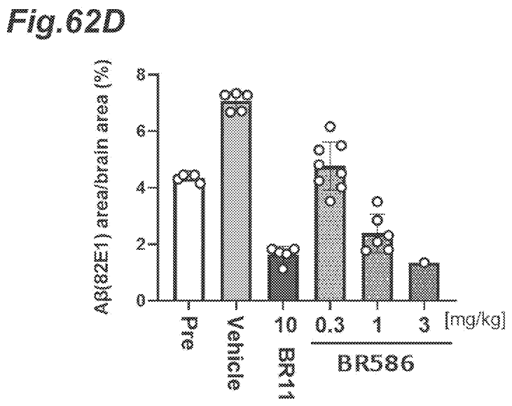

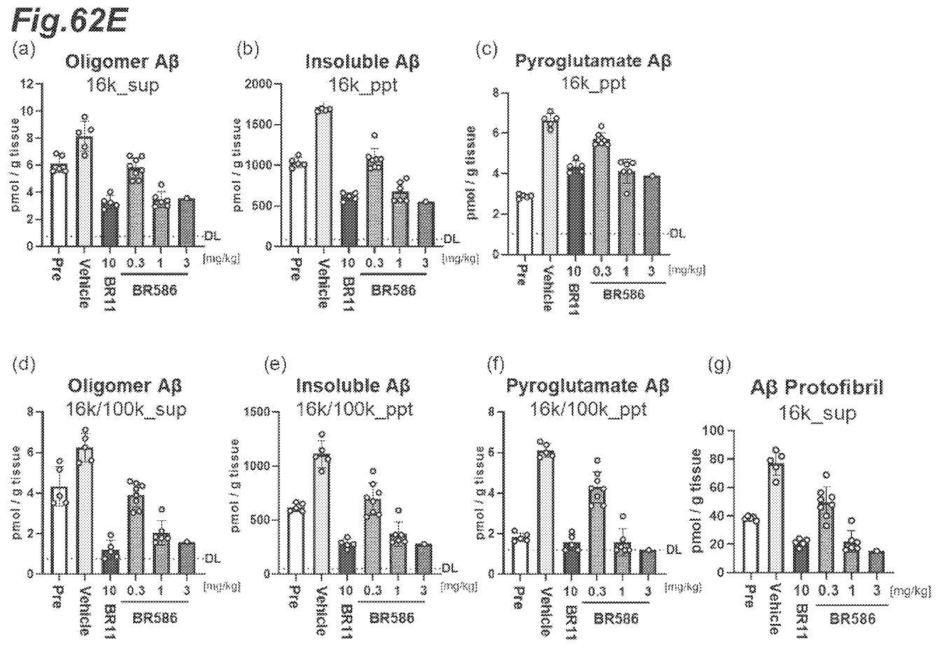

The measurement of plasma concentration of BR11 and BR586 was performed by ELISA as described below. A monomer Aβ 1-40 (Anaspec, Inc.) was added to MaxiSorp Plates (Nunc) and incubated overnight at 4° C. After blocking with 1% BSA/PBS at room temperature for one hour, the plates were washed with 0.02% Tween20/PBS three times and mouse plasma diluted in 0.1% BSA/0.02% Tween20/PBS was added and incubated at room temperature for one hour. After washing with 0.02% Tween20/PBS three times, an HRP-labeled goat anti-mouse IgG antibody (Jackson ImmunoResearch Inc.) was added and incubated at room temperature for one hour. After washing with 0.02% Tween20/PBS five times, TMB substrate solution (SeraCare) was added and incubated at room temperature for 10 minutes. An equivalent amount of 2N sulfuric acid was added to quench the reaction, and the absorbance at 450 nm was measured with a microplate reader SpectraMax (Molecular Devices, LLC). Calibration curves were generated based on the absorbance of standard samples at each concentration, and the antibody concentrations in the plasma were determined (FIG. 62A). The detection limit was set at 0.06 nM, and samples with concentrations below 0.06 nM were assigned a value of 0.06 nM. Samples with markedly low antibody concentration in plasma were excluded from the following analysis. The threshold of antibody concentrations set for each dosing group were as following: 0.15 nM (0.3 mg/kg group), 0.5 nM (1.0 mg/kg group), and 1.5 nM (3.0 mg/kg group).

### (4) Quantification of Phosphorylated Tau231 Area

Frozen brain sections (10 μm thickness) were prepared using a cryostat CM1860 (Leica) and mounted on a glass slide. After air drying, sections were treated with PBS and 0.1% Triton (trademark) X-100/PBS, each for 5 minutes at room temperature. After blocking with 0.1% Triton (trademark) X-100 1% normal donkey serum/1× Block Ace at room temperature for one hour, sections were incubated with Alexa488-labeled mouse anti-phosphorylated tau231 antibody (AT180) at room temperature for two hours. After washing with 0.1% Triton (trademark) X-100/PBS, mounting was performed with Prolong Glass Antifade Mountant (Thermo). Images were acquired using NanoZoomer (Hamamatsu Photonics) and the phosphorylated tau231 area was quantified using NIS Elements software (Nikon). The mean of phosphorylated tau23 1 area per brain area for each mouse was calculated, and the region of phosphorylated tau231 was quantified. The results of phosphorylated tau23 1 region are shown in FIG. 62B. Effects equivalent to the reduction in phosphorylated tau231 region, which were observed by the administration of BR11 at 10 mg/kg were observed with the bispecific anti-human TfR/Aβ antibody BR586 at lower doses.

### (5) Quantification of Amyloid Plaque Region

Frozen brain sections (10 μm thickness) were prepared using a cryostat CM1860 (Leica). After air drying, sections were treated with PBS and 0.1% Triton (trademark) X-100/PBS at room temperature for 5 minutes. After blocking with 0.1% Triton (trademark) X-100/1% normal donkey serum/1× Block Ace at room temperature for one hour, sections were incubated with Alexa555-labeled mouse anti-Amyloid antibody (82E1) at room temperature for two hours. After washing with 0.1% Triton (trademark) X-100/PBS, mounting was performed with Prolong Glass Antifade Mountant (Thermo). Images were acquired using NanoZoomer (Hamamatsu Photonics), signals of the 82E1 antibody were calculated as amyloid plaque area, using NIS Elements (Nikon Corporation). The average value for each mouse was calculated, and amyloid plaque was quantified (FIGS. 62C and 62D). Effects equivalent to the reduction in amyloid plaque, which are observed by the administration of BR11 at 10 mg/kg were observed with the bispecific anti-human TfR/Aβ antibody BR586 at lower doses.

### (6) Biochemical Quantification of Aβ in Brain Tissue

The right cerebral hemisphere collected in (2) was homogenized in ten volumes of TBS/complete (Roche) using a homogenizer (BRANSON). The homogenate was centrifuged at 16,000×g for 60 minutes at 4° C. The resulting supernatant and pellet were designated as 16k_sup and 16k_ppt, respectively. In addition, the 16k_sup was further ultracentrifuged at 100,000×g for 60 minutes at 4° C., and the supernatant after the centrifugation was referred to as 16k/100k_sup, and the precipitate was referred to as 16k/100k_ppt. For 16k_sup and 16k/100k_sup, the reaction was performed using Human Amyloidβ Oligomers (82E1-specific) Assay Kit (IBL Co., Ltd.) according to the instructions in the kit. After the reaction, the absorbance at 450 nm was measured with a microplate reader SpectraMax (Molecular Devices, LLC), and the amount of oligomer Aβ per g of wet brain tissue was calculated. For 16k_ppt and 16k/100k_ppt, the reaction was performed using human Amyloid beta (1-42) kit (FUJIFILM Wako Pure) and Human amyloidβ (N3pE-42) Assay Kit (IBL Co., Ltd.) according to the instructions in the kit. After the reaction, the absorbance at 450 nm was measured with a microplate reader SpectraMax (Molecular Devices, LLC), and the amounts of insoluble Aβ and pyroglutamate Aβ per g of wet brain tissue were calculated. The results of the oligomer Aβ, insoluble Aβ, and pyroglutamate Aβ are shown in FIG. 62E (a) to (f). For oligomer Aβ (16k_sup and 16k/100k_sup), insoluble Aβ (16k_ppt), insoluble Aβ (16k/100k_ppt), pyroglutamate Aβ (16k_ppt), and pyroglutamate Aβ (16k/100k_ppt), 0.76 μmol/g tissue, 89.43 μmol/g tissue, 52.17 μmol/g tissue, 1.03 μmol/g tissue, and 1.20 μmol/g tissue, respectively, were shown as the detection limits (DL). Individuals in which pyroglutamate Aβ (16k/100k_ppt) was less than the detection limit were shown as 1.20 μmol/g tissue. Effects equivalent to the reduction in oligomer Aβ, insoluble Aβ, and pyroglutamate Aβ, which are observed by the administration of anti-Aβ antibody BR11 at 10 mg/kg were observed with the bispecific anti-human TfR/Aβ antibody BR586 at lower doses.

### (7) Biochemical Quantification of Aβ Protofibril in Brain

The Aβ protofibril-capturing antibody BR10 was labeled onto magnetic beads to prepare antibody-labeled magnetic beads. The prepared antibody-labeled beads were diluted with Discovery/Assay Buffer (EMD Millipore) to a concentration of 100 μg/mL. Fifty microliters of the antibody-labeled bead solution and 150 μL of TBS-soluble fraction 16k_sup diluted with Discovery Standard Diluent (EMD Millipore) were added to each well of a 96-well V-bottom plate (Axygen), and the plate was shaken at 27° C. and 800 rpm for 2 hours. After washing the 96-well V-bottom plate with washing buffer, a fluorescent dye-labeled anti-Aβ antibody, mouse 3D6 antibody (ADx Neurosciences), was added to each well, followed by shaking at 27° C. and 800 rpm for 1 hour. After washing the 96-well V-bottom plate with washing buffer, 12 μL of Elution Buffer B (EMD Millipore) was added to each well, and the plate was shaken at 27° C. and 800 rpm for 10 minutes. After shaking, 10 μL was collected from each well of the plate and added to each well of a 384-well plate (Aurora Microplates) to which 10 μL of Buffer D (EMD Millipore) had been added in advance. The 384-well plate was shaken at 25° C. and 1000 rpm for 2 minutes, and fluorescence derived from the fluorescently labeled antibody was counted using SMCxPRO (EMD Millipore), and the Aβ protofibril concentration was calculated (FIG. 62E (g)). The quantification results were normalized to wet brain weight. An effect equivalent to the reduction in Aβ protofibril concentration observed with administration of 10 mg/kg of the anti-Aβ antibody BR11 was observed with lower doses of the bispecific anti-human TfR/Aβ antibody BR586.

### Experimental Example 63: Production of Monoclonal Antibodies (BR838 and BR839)

### (1) Generation of Stable Cell Lines Expressing Bispecific Anti-Human TfR/Aβ Mouse IgG2a Chimeric Antibody

The amino acid sequences encoding the human IgG1 constant region of BR802 and BR803 were replaced with amino acid sequences encoding the mouse IgG2a constant region (without N297G) to design mouse chimeric antibodies BR838 and BR839. These expression vectors were introduced into CHO cells by electroporation, and a pool of stable expressing cells was obtained by selective culture using MSX (L-Methionine-DL-sulfoximine), a glutamine synthetase inhibitor.

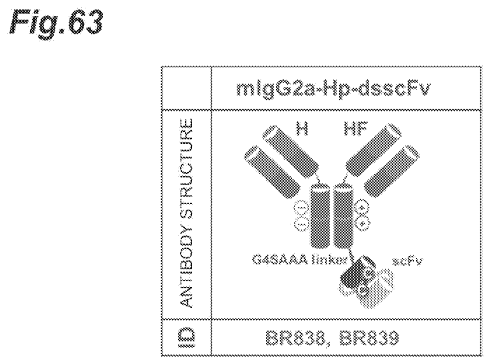

FIG. 63 shows schematic diagrams of BR838 and BR839, and Table 28 shows the combinations of the first and second heavy chains and light chains included in BR838 and BR839, respectively.

TABLE 28

| | | | DNA/ | SEQ |
|---|---|---|---|---|
| | | Antibody | Amino | ID |
| ID | Antibody name | region | acid | NO: |
| BR838 | DI 8-mlgG2a-Hp-HΔK- | First heavy | DNA | 303 |
| | aT04m02.1 dsscFv | chain (H) | Amino | 304 |
| | H; K409D, K439E, HΔK | | acid | |
| | HF; D356K, D399K, | Second heavy | DNA | 305 |
| | linker (G4SAAA), | chain (HF) | Amino | 306 |
| | aT04m02-dsscFv | and dsscFv | acid | |
| | L; wt | region | | |
| | | Light chain (L) | DNA | 307 |
| | | | Amino | 101 |
| | | | acid | |
| BR839 | DI 8-mlgG2a-Hp-HΔK- | First heavy | DNA | 303 |
| | aT04m0206.1 dsscFv | chain (H) | Amino | 304 |
| | H; K409D, K439E, HΔK | | acid | |
| | HF; D356K, D399K, | Second heavy | DNA | 308 |
| | linker (G4SAAA), | chain (HF) | Amino | 201 |
| | aT04m0206-dsscFv | and dsscFv | acid | |
| | L; wt | region | | |
| | | Light chain (L) | DNA | 307 |
| | | | Amino | 101 |
| | | | acid | |

### (2) Production and Purification of Bispecific Anti-Human TfR/Aβ Mouse IgG2a Chimeric Antibody

Stable expressing cells were cultured in a fed-batch manner, and the supernatant, obtained by removing cells, was affinity-purified using a MabSelect SuRe column (Cytiva, Inc.). The antibody was eluted with 100 mM citrate buffer (pH 3.8) containing 500 mM arginine hydrochloride, and then diluted 10-fold with 25 mM sodium acetate buffer (pH 5.6). Next, by-products and POI were separated using a cation exchange column, HiTrap Capto SP Impres (Cytiva). Fractions containing POI were collected, and aggregates were removed by gel filtration chromatography using Superdex 200 μg (Cytiva) carrier and buffer-exchanged with PBS.

### Experimental Example 64: Evaluation of Dose Reduction of a Bispecific Antibody (APP KI/hTfR-KI Mice)

### (1) Establishment of APP KI/hTfR KI (Homo/Hetero) Mice

Similarly to Experimental Example 57, APP-KI/hTfR-KI (homo/hetero) mice were generated.

(2) Sample Preparation from Animals Given Bispecific Anti-Human TfR/Aβ Mouse IgG2a Antibody

Male APP KI/hTfR-KI (homo/hetero) mice at 6 months of age were administered the anti-Aβ mouse IgG2a antibody BR11 at a dose of 1.0 mg/kg, 3.0 mg/kg, and 10 mg/kg, and the bispecific anti-human TfR/Aβ mouse IgG2a antibody BR838 at doses of 0.3 mg/kg, 1.0 mg/kg, and 3.0 mg/kg, calculated as an IgG equivalent amount, once a week for 12 weeks by intraperitoneal injection (10 mL/kg). PBS was administered to the control group. One week after the final administration, blood was collected under anesthesia, followed by exsanguination via incision of the right atrial appendage and transcardial perfusion with heparin-containing PBS. The brain was harvested and sectioned into left and right cerebral hemispheres. The left cerebral hemisphere was immersed in 1% paraformaldehyde (TAAB Laboratories Equipment Ltd.)/PBS and fixed at 4° C. for 24 hours. The fixed left cerebral hemisphere was sequentially replaced with 10% sucrose/PBS and 20% sucrose/PBS, and embedded in Tissue-Tek O. C. T. Compound (Leica) and frozen in liquid nitrogen to prepare a cryosection block. The right cerebral hemisphere was weighed to determine wet tissue weight, frozen in liquid nitrogen, and stored at −80° C. The collected blood was centrifuged at 1,200×g for 15 minutes at 4° C., and the supernatant was collected as plasma.

### (3) Measurement of Antibody Concentration in Plasma

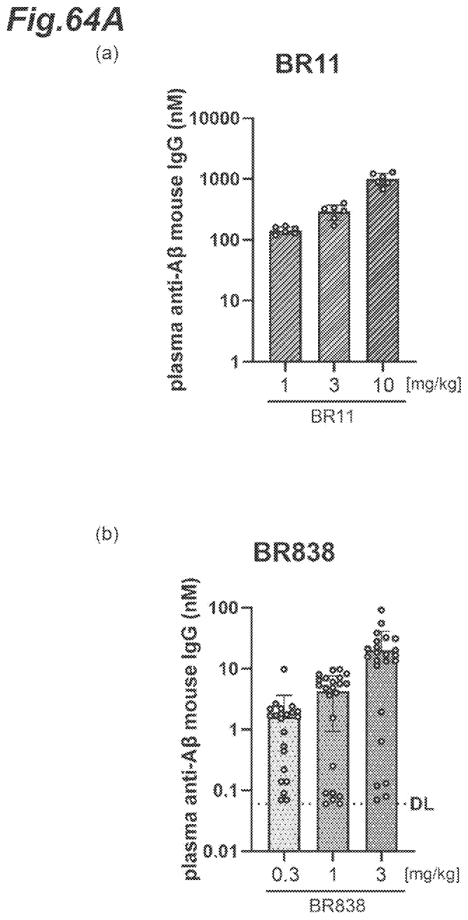

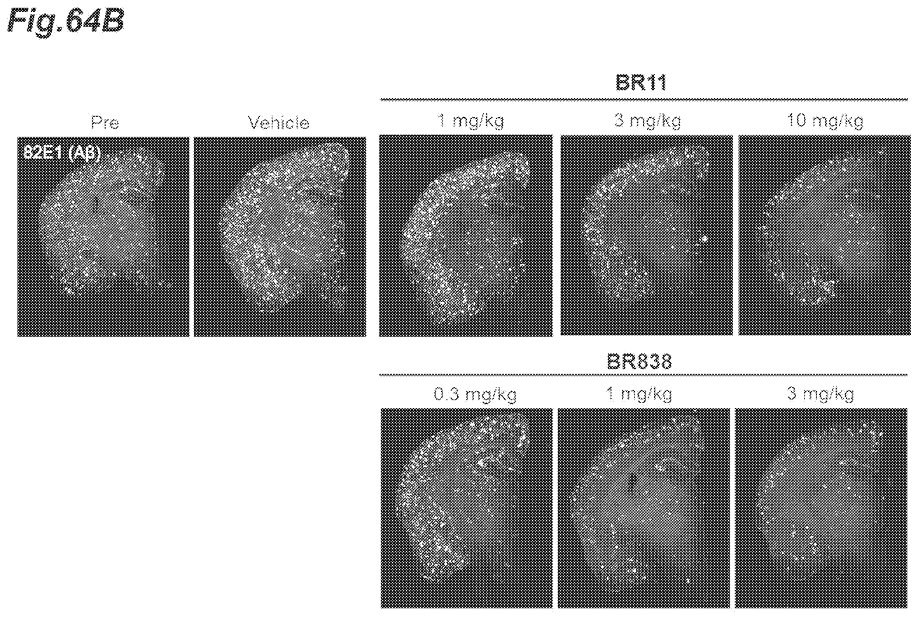

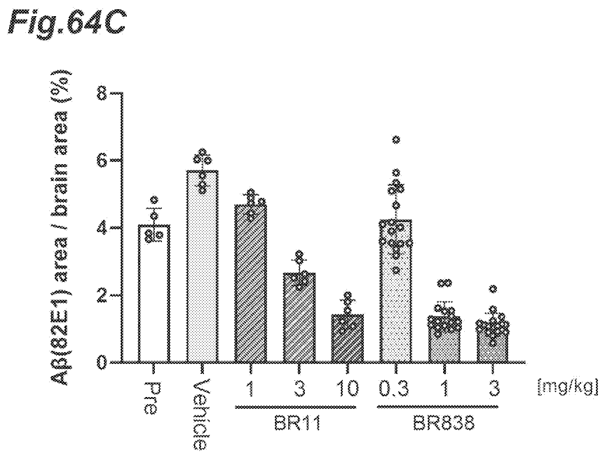

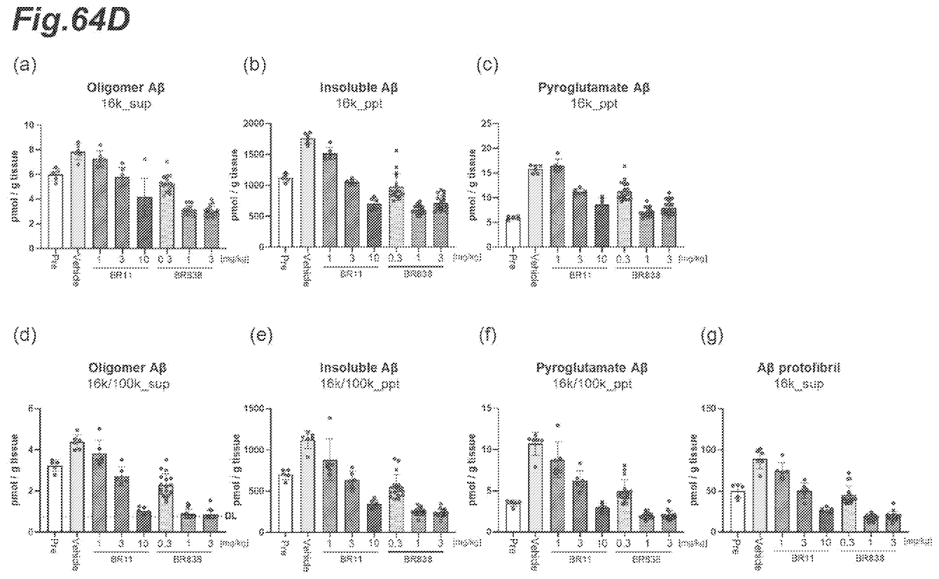

The measurement of plasma concentration of BR11 and BR838 was performed by ELISA as described below. A monomer Aβ 1-40 (Anaspec, Inc.) was added to MaxiSorp Plates (Nunc) and incubated overnight at 4° C. After blocking with 1% BSA/PBS at room temperature for one hour, the plates were washed with 0.02% Tween20/PBS three times and mouse plasma diluted with 0.1% BSA/0.02% Tween20/PBS was added to react at room temperature for one hour. After washing with 0.02% Tween20/PBS three times, an HRP-labeled goat anti-mouse IgG antibody (Jackson ImmunoResearch Inc.) was added and incubated at room temperature for one hour. After washing with 0.02% Tween20/PBS five times, TMB substrate solution (SeraCare) was added and incubated at room temperature for 10 minutes. An equivalent amount of 2N sulfuric acid was added to quench the reaction, and the absorbance at 450 nm was measured with a microplate reader SpectraMax (Molecular Devices, LLC). Calibration curves were generated based on the absorbance of standard samples at each concentration, and the antibody concentrations of the plasma samples were determined (FIG. 64A). The detection limit was set to 0.06 nM, and samples with concentrations below 0.06 nM were assigned a value of 0.06 nM. Samples with markedly low antibody concentration in plasma were excluded from the following analysis. The threshold of antibody concentrations of BR803 set for each dosing group was as following: 0.15 nM (0.3 mg/kg group), 0.5 nM (1.0 mg/kg group), and 1.5 nM (3.0 mg/kg group).

### (4) Quantification of Phosphorylated Tau231 Area

Frozen brain sections (10 μm thickness) are prepared using a cryostat CM1860 (Leica) and mounted on a glass slide. After air drying, sections are treated with PBS and 0.1% Triton (trademark) X-100/PBS, each for 5 minutes at room temperature. After blocking with 0.1% Triton (trademark) X-100 1% normal donkey serum/1× Block Ace at room temperature for one hour, sections are incubated with Alexa488-labeled mouse anti-phosphorylated tau231 antibody (AT180) at room temperature for two hours. After washing with 0.1% Triton (trademark) X-100/PBS, mounting is performed with Prolong Glass Antifade Mountant (Thermo). Images are acquired using NanoZoomer (Hamamatsu Photonics) and the phosphorylated tau231 area is quantified using NIS Elements software (Nikon). The mean of phosphorylated tau231 area per brain area for each mouse is calculated, and the area of phosphorylated tau231 is quantified. This experiment allows the evaluation of the effect of the bispecific anti-human TfR/Aβ antibody BR838 on phosphorylated tau231.

### (5) Quantification of Amyloid Plaque Region

Frozen brain sections (10 μm thickness) were prepared using a cryostat CM1860 (Leica) and mounted on a glass slide. After air drying, sections were treated with PBS and subsequently 0.1% Triton (trademark) X-100/PBS, each for 5 minutes at room temperature. After blocking with 0.1% Triton (trademark) X-100/1% normal donkey serum/1× Block Ace at room temperature for one hour, sections were incubated with Alexa555-labeled mouse anti-Amyloid antibody (82E1) at room temperature for two hours. After washing with 0.1% Triton (trademark) X-100/PBS, mounting was performed with Prolong Glass Antifade Mountant (Thermo). Images were acquired using NanoZoomer (Hamamatsu Photonics), signals of the 82E1 antibody were calculated as amyloid plaque area, using NIS Elements (Nikon Corporation). The average value for each mouse was calculated, and amyloid plaque was quantified. FIGS. 64B and 64C show the results of quantification of amyloid plaque. Effects equivalent to the reduction in amyloid plaque, which are observed by the administration of anti-Aβ antibody BR11 were observed with the bispecific anti-human TfR/Aβ antibody BR838 at lower doses.

### (6) Biochemical Quantification of Aβ in Brain Tissue

The right cerebral hemisphere collected in (2) was homogenized in ten volumes of TBS/complete (Roche) using a homogenizer (BRANSON). The homogenate was centrifuged at 16,000×g for 60 minutes at 4° C. The resulting supernatant and pellet were designated as 16k_sup and 16k_ppt, respectively. In addition, the 16k_sup was further ultracentrifuged at 100,000×g for 60 minutes at 4° C., and the supernatant after the centrifugation was referred to as 16k/100k_sup, and the precipitate was referred to as 16k/100k_ppt. For 16k_sup and 16k/100k_sup, the reaction was performed using Human Amyloidβ Oligomers (82E1-specific) Assay Kit (IBL Co., Ltd.) according to the instructions in the kit. After the reaction, the absorbance at 450 nm was measured with a microplate reader SpectraMax (Molecular Devices, LLC), and the amount of oligomer Aβ per g of wet brain tissue was calculated. For 16k_ppt and 16k/100k_ppt, the reaction was performed using human Amyloid beta (1-42) kit (FUJIFILM Wako Pure) and Human amyloidβ (N3pE-42) Assay Kit (IBL Co., Ltd.) according to the instructions in the kit. After the reaction, the absorbance at 450 nm was measured with a microplate reader SpectraMax (Molecular Devices, LLC), and the amounts of insoluble Aβ and pyroglutamate Aβ per g of wet brain tissue were calculated. The results of the oligomer Aβ, insoluble Aβ, and pyroglutamate Aβ are shown in FIG. 64D (a) to (f). For oligomer Aβ (16k/100k_sup), 0.76 μmol/g tissue was shown as the detection limits (DL). Individuals in which oligomer Aβ (16k/100k_sup) was less than the detection limit were shown as 0.76 pmol/g tissue. Effects equivalent to the reduction in oligomer Aβ, insoluble Aβ, and pyroglutamate Aβ which are observed by the administration of anti-Aβ antibody BR11 were observed with the bispecific anti-human TfR/Aβ antibody BR838 at a lower dose.

### (7) Biochemical Quantification of Ab Protofibril in Brain

The Aβ protofibril-capturing antibody BR10 was labeled onto magnetic beads to prepare antibody-labeled magnetic beads. The prepared antibody-labeled beads were diluted with Discovery/Assay Buffer (EMD Millipore) to a concentration of 100 μg/mL. Fifty microliters of the antibody-labeled bead solution and 150 μL of TBS-soluble fraction 16k_sup diluted with Discovery Standard Diluent (EMD Millipore) were added to each well of a 96-well V-bottom plate (Axygen), and the plate was shaken at 27° C. and 800 rpm for 2 hours. After washing the 96-well V-bottom plate with washing buffer, a fluorescent dye-labeled anti-Aβ antibody, mouse 3D6 antibody (ADx Neurosciences), was added to each well, followed by shaking at 27° C. and 800 rpm for 1 hour. After washing the 96-well V-bottom plate with washing buffer, 12 μL of Elution Buffer B (EMD Millipore) was added to each well, and the plate was shaken at 27° C. and 800 rpm for 10 minutes. After shaking, 10 μL was collected from each well of the plate and added to each well of a 384-well plate (Aurora Microplates) to which 10 μL of Buffer D (EMD Millipore) had been added in advance. The 384-well plate was shaken at 25° C. and 1000 rpm for 2 minutes, and fluorescence derived from the fluorescently labeled antibody was counted using SMCxPRO (EMD Millipore). The quantification results are shown in FIG. 64D (g). An effect equivalent to the reduction in Aβ protofibrils observed with administration of the anti-Aβ antibody BR11 was observed with lower doses of the bispecific anti-human TfR/Aβ antibody BR838.

### Experimental Example 65: Evaluation of ARIA Risk

The bispecific anti-human TfR/Aβ mouse IgG2a antibody BR838 or BR839 is administered to 5xFAD/hTfR-KI mice (or 5xFAD/hTfR-KI mice with the human APOE4 gene knocked in) for 8 weeks, and the number of infiltrating peripheral immune cells in the mouse brain tissue is quantified and analyzed, as in Experimental Example 15, thereby enabling the evaluation of the ARIA risk of BR838 or BR839.

(1) Sample Preparation from Animals Given Bispecific Anti-Human TfR/Aβ Mouse IgG2a Antibody

One week after the final administration, blood is collected under anesthesia, followed by exsanguination via heart, and the brain is harvested. The blood is centrifuged, and the supernatant is collected as plasma. TBS is added to the right cerebral hemisphere, tissue is homogenized, and the supernatant obtained after centrifugation is collected as TBS-soluble fraction. The left cerebral hemisphere is immersed and fixed, replaced with sucrose, and then embedded in O. C. T. Compound and frozen in liquid nitrogen.

### (2) Measurement of Plasma Concentration of Bispecific Anti-Human TfR/Aβ Mouse IgG2a Antibody

Antibody concentrations in plasma can be measured using an ELISA assay with immobilized monomeric Aβ1-40. After blocking, diluted plasma is added and detected with an HRP-conjugated secondary antibody. Color is developed with TMB substrate solution, and after the reaction is stopped, the absorbance at 450 nm is measured. The antibody concentration in the plasma sample is determined using a standard curve.

### (3) Amount of Oligomer Aβ in TBS Soluble Fraction

Oligomer Aβ is measured using Human Amyloidβ Oligomers (82E1-specific) Assay Kit according to the instructions in the kit. The absorbance at 450 nm is measured to determine the concentration of oligomer Aβ contained in the TBS-soluble fraction, and the amount of oligomeric Aβ per gram of wet brain tissue is calculated.

### (4) Evaluation of ARIA Risk Using Immune Cell Infiltration as Index

Sections of 10 μm thickness are prepared from the embedded frozen tissue and mounted on a glass slide. After washing with PBS, the tissue sections are subjected to blocking treatment and reacted with the diluted CD45 antibody and Iba1 antibody. After washing, a fluorescent-labeled secondary antibody is reacted to stain the cell nuclei. After final washing, the section is sealed with a sealing agent, and the fluorescent image is acquired. The number of CD45-positive Iba1-negative cells is used as an index for ARIA risk evaluation.

### Experimental Example 66: Preparation of Monoclonal Antibodies

In the vector expressing the second heavy chain and scFv genes of SEQ ID NO: 191 or 193 described in Experimental Example 40, the heavy chain variable region and the light chain variable region of aT04m02-scFv or aT04m0206-scFv are linked by a linker sequence. This linker sequence contains a sequence of two repeats ((GGGGS) 2) of an amino acid sequence consisting of four consecutive glycines followed by a serine (GGGGS (SEQ ID NO: 75); hereinafter sometimes referred to as “G4S”). In the present example, DNA sequences (SEQ ID NOs: 295 and 296) encoding a new second heavy chain and scFv having a linker sequence in which the two repeats ((GGGGS) 2) are replaced with four repeats ((GGGGS) 4) are synthesized. Vectors expressing a first heavy chain gene (amino acid sequence: SEQ ID NO: 189), the second heavy chain and scFv genes (amino acid sequences: SEQ ID NOs: 297 and 298), and a light chain gene (amino acid sequence: SEQ ID NO: 197) are then constructed to obtain BR834 and BR835. Hereinafter, in the antibody names shown in the tables, an scFv having a linker sequence consisting of a sequence (SEQ ID NO: 288) in which GGGG (SEQ ID NO: 77) is linked to the C-terminus of (GGGGS) 4 is referred to as “aT04m02.1 mv (4×G4S) G4” or “aT04m0206.1_mv_(4xG4S) G4”. Further, in the vector expressing the second heavy chain and scFv genes of BR834 or BR835, the glycine at the ninth residue from the N-terminus of HFR2 (heavy chain framework region 2) and the glycine at the third residue from the N-terminus of LFR4 (light chain framework region 4), as defined by Kabat numbering, are replaced with cysteine. Vectors expressing a new second heavy chain and dsscFv genes (DNA sequences: SEQ ID NOs: 299 and 300; amino acid sequences: SEQ ID NOs: 301 and 302) are constructed to obtain BR836 and BR837. Hereinafter, in the antibody names shown in the tables, a dsscFv having a linker sequence consisting of a sequence (SEQ ID NO: 288) in which GGGG (SEQ ID NO: 77) is linked to the C-terminus of (GGGGS) 4 is referred to as “aT04m02.1_dsscFv_(4xG4S) G4” or “aT04m0206.1_dsscFv (4×G4S) G4”.

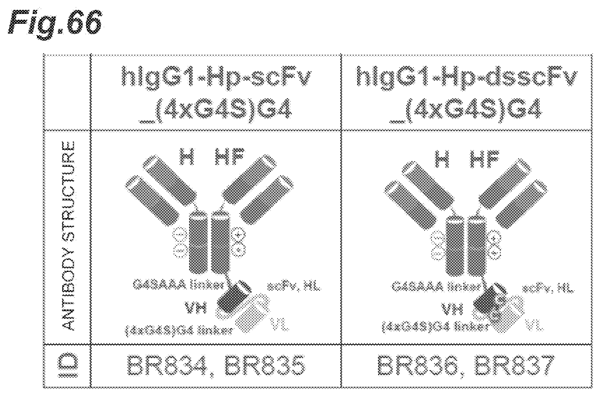

FIG. 66 shows schematic diagrams of each antibody, and Table 29 shows the combinations of the first and second heavy chains and light chains included in each of the antibodies BR834, BR835, BR836, and BR837, respectively.

TABLE 29

| | | | DNA/ | SEQ |
|---|---|---|---|---|
| | | Antibody | Amino | ID |
| ID | Antibody name | region | acid | NO: |
| BR834 | DI 8-hIgG1-N297G-Hp- | First heavy | DNA | 293 |
| | HΔK- | chain (H) | Amino | 189 |
| | aT04m02.1_mv_(4xG4S)G4 | | acid | |
| | H; N297G, K409D, K439E, | Second heavy | DNA | 295 |
| | HΔK | chain (HF) | | |
| | HF; N297G, D356K, D399K, | and dsscFv | Amino | 296 |
| | linker (G4SAAA), aT04m02- | region | acid | |
| | scFv | Light chain (L) | DNA | 294 |
| | L; wt | | Amino | 197 |
| | | | acid | |
| BR835 | DI 8-hIgG1-N297G-Hp- | First heavy | DNA | 293 |
| | HΔK- | chain (H) | Amino | 189 |
| | aT04m0206.1_mv_(4xG4S) | | acid | |
| | G4 | Second heavy | DNA | 297 |
| | H; N297G, K409D, K439E, | chain (HF) | Amino | 298 |
| | HΔK | and dsscFv | acid | |
| | HF; N297G, D356K, D399K, | region | | |
| | linker (G4SAAA), | Light chain (L) | DNA | 294 |
| | aT04m0206-scFv | | Amino | 197 |
| | L; wt | | acid | |
| BR836 | DI 8-hIgG1-N297G-Hp- | First heavy | DNA | 293 |
| | HΔK- | chain (H) | Amino | 189 |
| | aT04m02.1_dsscFv_(4xG4S) | | acid | |
| | G4 | Second heavy | DNA | 299 |
| | H; N297G, K409D, K439E, | chain (HF) | | |
| | HΔK | and dsscFv | Amino | 300 |
| | HF; N297G, D356K, D399K, | region | acid | |
| | linker (G4SAAA), aT04m02- | Light chain (L) | DNA | 294 |
| | dsscFv | | Amino | 197 |
| | L; wt | | acid | |
| BR837 | DI 8-hIgG1-N297G-Hp- | First heavy | DNA | 293 |
| | HΔK- | chain (H) | Amino | 189 |
| | aT04m0206.1_dsscFv_(4xG4 | | acid | |
| | S)G4 | Second heavy | DNA | 301 |
| | H; N297G, K409D, K439E, | chain (HF) | Amino | 302 |
| | HΔK | and dsscFv | acid | |
| | HF; N297G, D356K, D399K, | region | | |
| | linker (G4SAAA), | Light chain (L) | DNA | 294 |
| | aT04m0206-dsscFv | | Amino | 197 |
| | L; wt | | acid | |

Some embodiments of the inventions are follows.

[1]. A bispecific antibody comprising:
a first antigen-binding polypeptide that binds to amyloid beta (Aβ); and a second antigen-binding polypeptide that binds to human transferrin receptor (TfR),
wherein:
(i) the first antigen-binding polypeptide comprises a first heavy chain, a second heavy chain, and two light chains,
   wherein the first heavy chain and the second heavy chain each comprise a heavy chain variable region (VH) consisting of the amino acid sequence set forth in SEQ ID NO: 238,
wherein the first heavy chain and the second heavy chain each comprise a constant region,
   wherein the two light chains each comprise a light chain variable region (VL) consisting of the amino acid sequence set forth in SEQ ID NO: 239;
(ii) the second antigen-binding polypeptide comprises a heavy chain variable region comprising a heavy chain CDR1 (HCDR1) consisting of the amino acid sequence set forth in SEQ ID NO: 2, a heavy chain CDR2 (HCDR2) consisting of the amino acid sequence set forth in SEQ ID NO: 8 or SEQ ID NO: 13, and a heavy chain CDR3 (HCDR3) consisting of the amino acid sequence set forth in SEQ ID NO: 3, and a light chain variable region comprising a light chain CDR1 (LCDR1) consisting of the amino acid sequence set forth in SEQ ID NO: 4, a light chain CDR2 (LCDR2) consisting of the amino acid sequence set forth in SEQ ID NO: 5, and a light chain CDR3 (LCDR3) consisting of the amino acid sequence set forth in SEQ ID NO: 6; and
(iii) the second antigen-binding polypeptide is connected to a C-terminus of the constant region of the second heavy chain of the first antigen-binding polypeptide via a peptide linker.

[2]. The bispecific antibody according to [1], wherein the HCDR2 of the second antigen-binding polypeptide consists of the amino acid sequence set forth in SEQ ID NO: 8.

[3]. The bispecific antibody according to [2], wherein the heavy chain variable region of the second antigen-binding polypeptide consists of the amino acid sequence set forth in SEQ ID NO: 267 and the light chain variable region of the second antigen-binding polypeptide consists of the amino acid sequence set forth in SEQ ID NO: 265.

[4]. The bispecific antibody according to [1], wherein the HCDR2 of the second antigen-binding polypeptide consists of the amino acid sequence set forth in SEQ ID NO: 13.

[5]. The bispecific antibody according to [4], wherein the heavy chain variable region of the second antigen-binding polypeptide consists of the amino acid sequence set forth in SEQ ID NO: 275 and the light chain variable region of the second antigen-binding polypeptide consists of the amino acid sequence set forth in SEQ ID NO: 273.

[6]. The bispecific antibody according to [1], wherein the constant region of the first heavy chain of the first antigen-binding polypeptide and the constant region of the second heavy chain of the first antigen-binding polypeptide are human IgG1 heavy chain constant regions.

[7]. The bispecific antibody according to [1], wherein the constant region of the first heavy chain of the first antigen-binding polypeptide and the constant region of the second heavy chain of the first antigen-binding polypeptide are human IgG1 heavy chain constant regions comprising an N297G mutation at position 297 according to EU numbering.

[8]. The bispecific antibody according to [1], wherein:
the constant region of the first heavy chain of the first antigen-binding polypeptide is a human IgG1 heavy chain constant region comprising a K409D mutation at position 409 according to EU numbering and a K439E mutation at position 439 according to EU numbering; and
   the constant region of the second heavy chain of the first antigen-binding polypeptide is a human IgG1 heavy chain constant region comprising a D356K mutation at position 356 according to EU numbering and a D399K mutation at position 399 according to EU numbering.

[9]. The bispecific antibody according to [1], wherein the constant region of the first heavy chain of the first antigen-binding polypeptide is a human IgG1 heavy chain constant region in which a C-terminal lysine residue is deleted, and wherein the constant region of the second heavy chain of the first antigen-binding polypeptide is a human IgG1 heavy chain constant region.

[10]. The bispecific antibody according to [1], wherein the constant region of the first heavy chain of the first antigen-binding polypeptide comprises the amino acid sequence set forth in SEQ ID NO: 240, and the constant region of the second heavy chain of the first antigen-binding polypeptide comprises the amino acid sequence set forth in SEQ ID NO: 241.

[11]. The bispecific antibody according to [1], wherein a constant region of the light chain of the first antigen-binding polypeptide comprises the amino acid sequence set forth in SEQ ID NO: 119.

[12]. The bispecific antibody according to [1], wherein the second antigen-binding polypeptide comprises a dsscFv (disulfide-stabilized single-chain Fv) consisting of the amino acid sequence set forth in SEQ ID NO: 263.

[13]. The bispecific antibody according to [1], wherein the second antigen-binding polypeptide comprises a dsscFv (disulfide-stabilized single-chain Fv) consisting of the amino acid sequence set forth in SEQ ID NO: 271.

[14]. The bispecific antibody according to [1], wherein the peptide linker consists of the amino acid sequence set forth in SEQ ID NO: 79.

[15]. A bispecific antibody that binds to amyloid beta (Aβ) and human transferrin receptor (TfR), wherein
the bispecific antibody comprises a first polypeptide consisting of the amino acid sequence set forth in SEQ ID NO: 189, a second polypeptide consisting of the amino acid sequence set forth in SEQ ID NO: 269, and a third polypeptide consisting of the amino acid sequence set forth in SEQ ID NO: 197.

[16]. A bispecific antibody that binds to amyloid beta (Aβ) and human transferrin receptor (TfR), wherein
the bispecific antibody comprises a first polypeptide consisting of the amino acid sequence set forth in SEQ ID NO: 189, a second polypeptide consisting of the amino acid sequence set forth in SEQ ID NO: 277, and a third polypeptide consisting of the amino acid sequence set forth in SEQ ID NO: 197.

[17]. An isolated nucleic acid encoding the bispecific antibody according to [1].

[18]. A vector comprising the nucleic acid according to [17].

[19]. A host cell comprising the nucleic acid according to [17].

[20]. A method for producing a bispecific antibody, comprising a step of culturing the host cell according to [19].

[21]. A pharmaceutical composition comprising the bispecific antibody according to [1].

[22]. A method of treating an amyloid beta (Aβ)-related disease, comprising administering an effective amount of the bispecific antibody according to [1 to a human subject in need thereof.

[23]. The method of treating according to [22], wherein the Aβ-related disease is Alzheimer's disease (AD), mild cognitive impairment due to AD (MCI due to AD), preclinical AD, or Down's syndrome.

[24]. An isolated nucleic acid encoding the bispecific antibody according to [16].

[25]. A vector comprising the nucleic acid according to [24]. [26]. A host cell comprising the nucleic acid according to [24].

[27]. A method for producing a bispecific antibody, comprising a step of culturing the host cell according to [26].

[28]. A pharmaceutical composition comprising the bispecific antibody according to [16].

[29]. A method of treating an amyloid beta (Aβ)-related disease, comprising administering an effective amount of the bispecific antibody according to [16] to a human subject in need thereof.

[30]. The method of treating according to [29], wherein the Aβ-related disease is Alzheimer's disease (AD), mild cognitive impairment due to AD (MCI due to AD), preclinical AD, or Down's syndrome.

1.-37. (canceled)

38. A bispecific antibody comprising:
a first antigen-binding polypeptide that binds to amyloid beta (Aβ); and
a second antigen-binding polypeptide that binds to human transferrin receptor (TfR),
wherein:
(i) the first antigen-binding polypeptide comprises a first heavy chain, a second heavy chain, and two light chains,
wherein the first heavy chain and the second heavy chain each comprise a heavy chain variable region (VH) consisting of the amino acid sequence set forth in SEQ ID NO: 238,
wherein the first heavy chain and the second heavy chain each comprise a constant region,
wherein the two light chains each comprise a light chain variable region (VL) consisting of the amino acid sequence set forth in SEQ ID NO: 239;
(ii) the second antigen-binding polypeptide comprises a heavy chain variable region comprising a heavy chain CDR1 (HCDR1) consisting of the amino acid sequence set forth in SEQ ID NO: 2, a heavy chain CDR2 (HCDR2) consisting of the amino acid sequence set forth in SEQ ID NO: 8 or SEQ ID NO: 13, and a heavy chain CDR3 (HCDR3) consisting of the amino acid sequence set forth in SEQ ID NO: 3, and a light chain variable region comprising a light chain CDR1 (LCDR1) consisting of the amino acid sequence set forth in SEQ ID NO: 4, a light chain CDR2 (LCDR2) consisting of the amino acid sequence set forth in SEQ ID NO: 5, and a light chain CDR3 (LCDR3) consisting of the amino acid sequence set forth in SEQ ID NO: 6; and
(iii) the second antigen-binding polypeptide is connected to a C-terminus of the constant region of the second heavy chain of the first antigen-binding polypeptide via a peptide linker.

39. The bispecific antibody according to claim 38, wherein the HCDR2 of the second antigen-binding polypeptide consists of the amino acid sequence set forth in SEQ ID NO: 8.

40. The bispecific antibody according to claim 39, wherein the heavy chain variable region of the second antigen-binding polypeptide consists of the amino acid sequence set forth in SEQ ID NO: 267 and the light chain variable region of the second antigen-binding polypeptide consists of the amino acid sequence set forth in SEQ ID NO: 265.

41. The bispecific antibody according to claim 38, wherein the HCDR2 of the second antigen-binding polypeptide consists of the amino acid sequence set forth in SEQ ID NO: 13.

42. The bispecific antibody according to claim 41, wherein the heavy chain variable region of the second antigen-binding polypeptide consists of the amino acid sequence set forth in SEQ ID NO: 275 and the light chain variable region of the second antigen-binding polypeptide consists of the amino acid sequence set forth in SEQ ID NO: 273.

43. The bispecific antibody according to claim 38, wherein the constant region of the first heavy chain of the first antigen-binding polypeptide and the constant region of the second heavy chain of the first antigen-binding polypeptide are human IgG1 heavy chain constant regions.

44. The bispecific antibody according to claim 38, wherein the constant region of the first heavy chain of the first antigen-binding polypeptide and the constant region of the second heavy chain of the first antigen-binding polypeptide are human IgG1 heavy chain constant regions comprising an N297G mutation at position 297 according to EU numbering.

45. The bispecific antibody according to claim 38, wherein:
the constant region of the first heavy chain of the first antigen-binding polypeptide is a human IgG1 heavy chain constant region comprising a K409D mutation at position 409 according to EU numbering and a K439E mutation at position 439 according to EU numbering; and
the constant region of the second heavy chain of the first antigen-binding polypeptide is a human IgG1 heavy chain constant region comprising a D356K mutation at position 356 according to EU numbering and a D399K mutation at position 399 according to EU numbering.

46. The bispecific antibody according to claim 38, wherein the constant region of the first heavy chain of the first antigen-binding polypeptide is a human IgG1 heavy chain constant region in which a C-terminal lysine residue is deleted, and wherein the constant region of the second heavy chain of the first antigen-binding polypeptide is a human IgG1 heavy chain constant region.

47. The bispecific antibody according to claim 38, wherein the constant region of the first heavy chain of the first antigen-binding polypeptide comprises the amino acid sequence set forth in SEQ ID NO: 240, and the constant region of the second heavy chain of the first antigen-binding polypeptide comprises the amino acid sequence set forth in SEQ ID NO: 241.

48. The bispecific antibody according to claim 38, wherein a constant region of the light chain of the first antigen-binding polypeptide comprises the amino acid sequence set forth in SEQ ID NO: 119.

49. The bispecific antibody according to claim 38, wherein the second antigen-binding polypeptide comprises a dsscFv (disulfide-stabilized single-chain Fv) consisting of the amino acid sequence set forth in SEQ ID NO: 263.

50. The bispecific antibody according to claim 38, wherein the second antigen-binding polypeptide comprises a dsscFv (disulfide-stabilized single-chain Fv) consisting of the amino acid sequence set forth in SEQ ID NO: 271.

51. The bispecific antibody according to claim 38, wherein the peptide linker consists of the amino acid sequence set forth in SEQ ID NO: 79.

52. A bispecific antibody that binds to amyloid beta (Aβ) and human transferrin receptor (TfR), wherein
the bispecific antibody comprises a first polypeptide consisting of the amino acid sequence set forth in SEQ ID NO: 189, a second polypeptide consisting of the amino acid sequence set forth in SEQ ID NO: 269, and a third polypeptide consisting of the amino acid sequence set forth in SEQ ID NO: 197.

53. A bispecific antibody that binds to amyloid beta (Aβ) and human transferrin receptor (TfR), wherein
the bispecific antibody comprises a first polypeptide consisting of the amino acid sequence set forth in SEQ ID NO: 189, a second polypeptide consisting of the amino acid sequence set forth in SEQ ID NO: 277, and a third polypeptide consisting of the amino acid sequence set forth in SEQ ID NO: 197.

54. An isolated nucleic acid encoding the bispecific antibody according to claim 38.

55. A vector comprising the nucleic acid according to claim 54.

56. A host cell comprising the nucleic acid according to claim 54.

57. A method for producing a bispecific antibody, comprising a step of culturing the host cell according to claim 56.

58. A pharmaceutical composition comprising the bispecific antibody according to claim 38.

59. A method of treating an amyloid beta (Aβ)-related disease, comprising administering an effective amount of the bispecific antibody according to claim 38 to a human subject in need thereof.

60. The method of treating according to claim 59, wherein the Aβ-related disease is Alzheimer's disease (AD), mild cognitive impairment due to AD (MCI due to AD), preclinical AD, or Down's syndrome.

61. An isolated nucleic acid encoding the bispecific antibody according to claim 53.

62. A vector comprising the nucleic acid according to claim 61.

63. A host cell comprising the nucleic acid according to claim 61.

64. A method for producing a bispecific antibody, comprising a step of culturing the host cell according to claim 63.

65. A pharmaceutical composition comprising the bispecific antibody according to claim 53.

66. A method of treating an amyloid beta (Aβ)-related disease, comprising administering an effective amount of the bispecific antibody according to claim 53 to a human subject in need thereof.

67. The method of treating according to claim 66, wherein the Aβ-related disease is Alzheimer's disease (AD), mild cognitive impairment due to AD (MCI due to AD), preclinical AD, or Down's syndrome.
